(19) United States
(12) Patent Application Publication
(10) Pub. No.: US 2017/0051286 A1
(43) Pub. Date: Feb. 23, 2017

(54) METHODS AND MODIFICATIONS THAT PRODUCE ssRNAi COMPOUNDS WITH ENHANCED ACTIVITY, POTENCY AND DURATION OF EFFECT

(71) Applicant: Larry J. Smith, Omaha, NE (US)

(72) Inventor: Larry J. Smith, Omaha, NE (US)

(21) Appl. No.: 15/308,235

(22) PCT Filed: May 1, 2015
(86) PCT No.: PCT/US15/28958

§ 371 Date: Nov. 1, 2016

Related U.S. Application Data
(60) Provisional application No. 61/987,448, filed on May 1, 2014.

(51) Int. Cl. C12N 15/113; C12N 15/11

(52) U.S. Cl. CPC C12N 15/113; C12N 15/111; C12N 2310/14; C12N 2310/344; C12N 2310/321; C12N 2310/32; C12N 2310/315; C12N 2310/346

(57) ABSTRACT

Compositions and methods for down modulating expression of target nucleic acids using a single strand oligoribonucleotide siRNA compound are disclosed.

This application claims priority to U.S. Provisional Application No. 61/987,448 filed May 1, 2014, the entire contents being incorporated by reference herein.

### FIELD OF THE INVENTION

This invention relates to the fields of RNAi, oligonucleotide based therapeutics, medicine, drug development and functional genomics by providing novel means to modulate nucleic acid expression and/or function, particularly mRNA as well as coding and non-coding regulatory RNA. More specifically, the invention provides novel RNAi guide strands comprising modifications that enhance interactions with the RNAi mechanism within the cell and methods of use thereof for modulating expression of target genes and nucleic acids of interest.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

RNA interference (RNAi) refers to a set of overlapping cellular mechanisms (RNAi mechanisms) typically involvingin double stranded RNA (dsRNA) structures (RNAi triggers) which activate and direct the RNAi mechanism to particular nucleic acid targets on the basis of complementary base pairing. The RNAi mechanism then causes a modulation of the expression and/or function of the target(s). In most instances the nucleic acid targets are RNA (often mRNA) but coding and non-coding regulatory RNA can also be targeted. In addition, some RNAi triggers can direct an RNAi mechanism to certain DNA targets, such as the enhancer, silencer and/or promoter elements of particular genes, resulting in a modulation of the activity of the gene.

RNAi triggers are typically in the range of 21-23 nucleosides in length. Only one of these strands, called the guide strand or antisense strand, directs the RNAi mechanism to the target(s). The complementary strand to the guide strand in the RNAi trigger is called the passenger or sense strand. The passenger strand is discarded during the activation of the RNAi mechanism.

Two main categories of RNAi triggers have been distinguished: small inhibitory RNA (siRNA) and microRNA (miRNA). Both of these are generated from longer precursor dsRNA by the enzymatic capability of Dicer. In the case of siRNA, the original source of the dsRNA can be exogenous to the cell or it can be derived from transposable elements or certain viruses within the cell. In contrast, miRNA is produced from precursor molecules that are typically generated from independent genes or from intron sequences. Unlike siRNA, miRNA broadly inhibits multiple different targets, typically different mRNAs.

The differences between siRNA and miRNA are reflected, in part, by the specific “targeting codes” that are associated with them. The targeting code can be briefly defined as the subset of the guide strand sequence that directs the RNAi mechanism to the target to be engaged and then modulated. Ambros et al. (RNA 9: 277-9, 2003) and Griffiths-Jones et al. (Nucleic Acids Res 36: D154-8, 2007) provide a more detailed description of how naturally occurring siRNA and miRNA can be distinguished and annotated.

The general RNAi mechanisms that underlie the implementation of siRNA and miRNA based effects are substantially overlapping, but the particulars of how siRNA vs. miRNA based RNAi mechanisms modulate target expression and/or function are substantially different. At the heart of the general mechanisms applicable to both of these types of RNAi trigger is the RNA-induced silencing complex (RISC). The siRNA or miRNA is loaded into RISC typically with the help of the RISC loading complex (RLC) that commonly includes Dicer. During the activation of the RNAi mechanism by an RNAi trigger, the passenger strand is discarded and the guide strand is used to direct RISC to its target(s).

A guide strand that is separated from the passenger strand is non-covalently bound throughout most or all of its length to a member of RISC that is typically one of four argonaute proteins (AGO-1-4). Any argonaute protein can engage any RNAi trigger guide strand, miRNA or siRNA, but in humans only AGO-2 has intrinsic enzymatic activity which can cleave the RNA target. Guide strands from siRNA triggers engaged by AGO-2 or another argonaute can also result in target modulation by inducing a steric hindrance effect rather than target cleavage. Further, any of the argonautes can engage a miRNA guide strand that subsequently directs RISC to its targets. Tuschl's group first reported the administration of synthetic siRNA against mRNA targets of choice to cells (Elbashir et al., Nature 411: 494-498, 2001). This success lead to a greatly increased interest in manufacturing siRNA drugs based on natural RNAi triggers to treat a variety of diseases. The continuing bottleneck that prevents the broad use of such drugs in the clinic, however, is their requirement for a carrier that can greatly increase the efficiency with which the double strand siRNA or miRNA drug crosses the surface membrane of cells in the body. Despite a great effort there is still an absence of any individual or any set of carriers capable of achieving this for a wide variety of tissues and cell types (Aliabadi et al., Biomaterials 33: 2546-69, 2012; Kanasty et al., Mol Ther 20: 513-24, 2012).

Administration of siRNA and miRNA drugs, however, has very recently reached the stage where a therapeutically useful level of RNAi activity can be achieved in the livers of humans. Given the natural function of liver that involves the uptake, processing and elimination of a variety of substances from the body, the liver is particularly well suited compared to other organs/tissue/cell types for carrier-mediated uptake of large drug molecules such as double strand RNAi drugs.

miRNA is a fundamentally more complex area of RNAi than siRNA and consequently, attempts to acquire miRNA candidates for therapeutic use have lagged behind. Potential miRNA-related therapeutics includes miRNA inhibitors and miRNA mimics. Most advanced is the use of antisense oligos with a steric hindrance mechanism to inhibit the function of certain miRNAs. Since these antisense oligos are single stranded, they do not require a carrier to get into cells in the body and inhibitory activity is not limited to the liver. One example is a mixed LNA/DNA phosphorothioate oligo that inhibits miR-122. It has completed phase II testing with promising results (Janssen et al. N Engl J Med 368: 1685-94, 2013). miR-122 is highly expressed by liver and is required for HCV production and it increases the level of total cholesterol in plasma. Steric hindrance antisense oligos require higher doses and higher affinity for their target compared to RNase H1 dependent antisense oligos or to siRNA (Elmen et al., Nature 452: 896, 2008; Lanford et al., Science 327: 198, 2010).

One solution to the inability to get double strand RNAi drugs into the various tissue/cell types in the body is to use single strand RNAi drugs. This has been achieved by sequentially administering drugs (seqRNAi) to subjects that correspond to the passenger and guide strands of double strand drugs (WO 2011/046983; WO 2012/145729). Another possibility is the use of guide strand only drugs (ssRNAi). These would have the advantage of having simpler dosing schedules that do not require the administration of a different second drug, corresponding to the passenger strand, in order to obtain the desired RNAi effect on the target in the body.

The compositions, methods and uses for providing the best ssRNAi compositions known in the art for in vitro and in particularly for in vivo use, however, have shortcomings, such as inadequate potency, that hinder their clinical and commercial use. These compositions, methods and uses (newer ssRNAi) are described in Haringsma et al., Nucleic Acids Res 40: 4125-37, 2012; Yu et al., Cell 150: 895-908, 2012; Chorn et al., RNA 18: 96-804, 2012; Lima et al., Cell 150: 883-94, 2012; WO 2011/046983; WO 2011/139699; WO 2011/139702; and WO 2012/027206. In addition, possible alternatives to the 5′-VP phosphate analog conjugated to the 5′end terminal nucleoside in the ssRNAi compounds found in Lima et al. (2012) and Yu et al., (2012) were evaluated by the same group and reported in Prakash et al., Nucleic Acids Res 43: 2993-3011, 2015. None of the tested alternatives to 5′-VP by Prakash et al., however, provided a better outcome against the intended target compared to compounds with 5′-VP when the various ss-siRNAs were evaluated in mice.

Theses compounds are, however, a substantial improvement over the ssRNAi previously known in the art in that they allow effective ssRNAi compounds to be routinely generated for a variety of targets. This was achieved by providing for adequate basic nuclease resistance in the ssRNAi in a manner that does not eliminate the possibility of obtaining RNAi activity.

The best of these newer ssRNAi compounds require an enveloping carrier for added nuclease protection in extracellular fluids when used in animals and were generated by Haringsma et al., (Nucleic Acids Res 40: 4125-37, 2012) and Chorn et al., (RNA 18: 96-804, 2012). These compounds are 21-mers that have the following modifications: (1) A phosphate conjugated to the 5′ carbon of the 5′-end nucleoside sugar (5′-phosphate); (2) 2′-O-methyl (2′-O-Me) uridines in nucleotide positions 20 and 21; (3) 2′-fluoros in positions 1-19; and (4) Only phosphodiester linkages.

Using this modification pattern two ssRNAi compounds targeting different sites on Apo-B mRNA (sequences 8786 and 6981) and their cognate siRNAs were transfected into mouse Hepa1-6 cells at different doses, by Haringsma et al., to generate dose response curves. The cells were harvested 24 hours after the transfection and the target mRNA levels determined. The 8786 siRNA had an IC₅₀ value of 15 pM vs. an IC₅₀ value of about 1.2 nM for the ssRNAi while the 6981 siRNA had an IC₅₀ value of 45 pM vs. an IC₅₀ value of about 0.9 nM for the ssRNAi. These values respectively represent an 80× and a 20× greater potency for the siRNA over the cognate ssRNAi. The activity of all 4 of these compounds plateaued at >90% target knockdown showing that ssRNAi can be as active as a cognate siRNA but that it is much less potent than the siRNA.

Haringsma et al. also compared the activity, potency and duration of effect of the 8786 and 6981 ssRNAi compounds compared to their cognate siRNA where the compounds were delivered to mouse liver cells in vivo using a protective lipid nanoparticle carrier. As for the in vitro experiments, the siRNA passenger strands were unmodified except for single inverted abasic modifications at the 5′ and 3′-ends. When the two cognate compound types were compared at a dose of 3 mg/kg on day 3 following the i.v. treatment of mice, the two ssRNAi compounds were shown to produce a knockdown of the Apo-B mRNA target in the range of 61-74% while the two siRNAs produced a knockdown in the range of 85-98% indicating that the ssRNAi compounds are less potent than the cognate siRNA in mice at this dose. Note that giving the ssRNAi and siRNA at the same drug weight means that the dose of ssRNAi will supply twice as many guide strand to cells as the siRNA dose does.

A more striking difference between these two sets of cognate compounds is that the ssRNAi compound in each set had a much shorter duration of suppressive activity against the target compared to the cognate siRNA. In these experiments all 4 compounds were administered at a dose of 6 mg/kg and the target mRNA levels in mouse liver were determined on days 2, 7 and 14. Throughout this time course administration of either of the siRNA compounds resulted in a consistent level of target knock down in the liver of about 95% while the two ssRNAi compounds showed a nearly a 90%, 71-77% and no knockdown respectively at each of these time points. In addition, the siRNA but not the ssRNAi compounds resulted in a cleavage of the target mRNA at the definitive site for siRNA induced cleavage as determined by the 5′-RACE assay.

These data make it clear that the ssRNAi compounds have a lower potency and a much shorter duration of effect on the target compared to the cognate siRNA. In addition, unlike siRNA, ssRNAi compounds fail to induce cleavage of the target at the expected AGO-2 cleavage site (the site in the target opposite nucleoside positions 10 and 11 of the guide strand), but other considerations supported an RNAi mechanism for the effect of these ssRNAi compounds.

In the absence of argonaute protein engagement, ssRNAi, are less protected from nuclease attack than the guide strand in a duplex with its passenger strand. For this reason the initial difference in activity between the two compositions used in the Haringsma et al. experiments on day 2 or 3 could simply reflect a greater level of degradation of the ssRNAi compared to the siRNA guide strand in cells. But since the guide strands in both compounds are the same, the differences subsequently seen in Apo-B mRNA levels and the different 5′RACE results cannot be explained on the basis of differences in the guide strand sequence or modifications. Instead these differences in effects on the target between the ssRNAi and siRNA must be due to differences in the quality of the interactions between the ssRNAi and siRNA guide strands and RISC, particularly with regard to the argonaute component of RISC that is in direct contact with the guide strand throughout all or nearly all of its length.

Chorn et al. (RNA 18: 96-804, 2012) applied the same set of modifications as Haringsma et al., to ssRNAi 21-mer mimics of miR-124 and miR-122. They transfected these compounds into HCT-116 cells and measured the levels of two mRNA targets each for the miRNAs being mimicked.

Since the targeting code for these and most other miRNA guide strands is primarily, if not exclusively, the seed sequence the rest of the guide strand sequence can be largely altered at will. Taking advantage of this Chorn et al., also evaluated the activity of 88 ssRNAi mimics for which the bases (A, U, C or G) found in nucleoside positions 10-19 were randomized. Several of the ssRNAi compounds with a randomly generated partial sequence were more active than an ssRNAi having the full endogenous miR-124 guide strand sequence. A significant positive correlation was seen between the pyrimidine content of the randomized segment of the ssRNAi and its activity. Not every high pyrimidine content randomized sequence, however, was a top performer.

A randomized sequence from one of the best performing miR-124 mimics was shown to be capable of significantly boosting the activity of an ssRNAi mimic of miR-122 compared to an ssRNAi with the full miR-122 sequence. The IC₅₀ for the optimized miR-122 mimic was in the low single digit nanomolar range while the ssRNAi based on the full miR-122 sequence exclusive of the endogenous overhang had no activity at doses lower than 10 nM. Extrapolation of the dose response curve suggests that the IC₅₀ for the latter ssRNAi was in the range of 100 nM.

Lima et al., (Cell 150: 883-94, 2012) generated newer 21-mer ssRNAi compounds with sufficient nuclease resistance to be systemically administered to mice without a protective carrier. They also had enough phosphorothioate linkages, although not an optimal number, to promote sufficient binding to plasma proteins to prevent the majority of the compound from being rapidly eliminated from the body by the kidneys rather than being taken up by tissues from the general circulation. The modifications used to promote nuclease resistance for the most potent of these ssRNAi compounds were as follows: (1) A 5′-VP-Tₘₒₑ at the 5′-end. Tₘₒₑ is a thymidine with a 2′-methoxyethyl (2′-MOE) modification of the sugar. 5′-VP stands for 5′-(E)-vinylphosphonate a moiety that contains a double bond between nucleoside sugar carbons 5′ and 6′ in a trans configuration. It is a 5′-phosphate analog with a double bond that is expected to be resistant to phosphatases; (2) Nucleoside positions 20 and 21 were both adenosines with the 2′-MOE modification (Aₘₒₑ); (3) Nucleoside positions 2-19 were alternating 2′-fluoro and 2′-O-methyl where a 2′-fluoro falls in position 14; and (4) Phosphorothioate linkages were used between consecutive nucleoside positions 1-3 and 14-21. In nucleoside positions 3-14 phosphodiester linkages alternate with phosphorothioate bringing the total number of phosphorothioates in the ssRNAi to 14. The synthesis methods for generating of oligos with 5′-VP include those described in WO 2011/139699, WO 2011/139702, Lima et al., (Cell 150: 883-94, 2012) and Prakash et al., (Nucleic Acids Res 43: 2993-3011, 2015).

Lima et al. used electroporation to get varying doses of ssRNAi 21 or ssRNAi 8 into mouse primary hepatocytes to determine the IC₅₀ for their target, PTEN mRNA, 16 hours post electroporation. These ssRNAi compounds had the same sequence and an identical pattern of modification as just described except ssRNAi 21 had the 5′-VP-Tₘₒₑ modification while ssRNAi 8 had a normal phosphate group conjugated to the 5′ carbon of the Tₘₒₑ sugar rather than VP. The results showed IC₅₀ values of 1104 for ssRNAi 21 and 1 μM for ssRNAi 8. This indicates that the compound with a 5′ normal phosphate is about 10× more potent than the compound with the 5′ phosphate analog in primary mouse hepatocytes.

Cells are known to vary widely in their susceptibility to transfection so the very high IC₅₀ doses for both of these ssRNAi compounds could be due to a poor susceptibility to transfection on the part of this cell type. This possibility is supported by the fact that the IC₅₀ for ssRNAi 8 when Hela cells were used was 2±0.4 nM indicative of a 500× greater potency for this compound compared to its IC₅₀ based on primary mouse hepatocytes.

Mice were treated with ssRNAi 8 at a total dose of 300 mg/kg and the effect on mouse liver PTEN mRNA levels determined. (For this and the other experiments described below, the ssRNAi was delivered as a divided total dose where 25 mg/kg was given twice a day s.c. for the required number of days needed to reach the total dose.) There was no effect of ssRNAi 8 on the mRNA target. Recovery of the ssRNAi from the liver showed that the 5′-phosphate was no longer present on the ssRNAi after being in liver cells for 6 hours while the rest of the strand was intact. Lima et al. reasoned that the lack of activity against the target was due to this phosphate loss. This finding stands in marked contrast to the finding of Haringsma et al. (2012) where their ssRNAi compound with a natural 5′-end phosphate was active in mouse liver over a period of several days.

Given the 10-fold lower potency of ssRNAi 21 compared to ssRNAi 8 in primary hepatocytes additional ssRNAi sequences with the 5′-VP-Tₘₒₑ modification were tested for their ability to suppress PTEN mRNA. Two with a level of activity (IC₅₀ of 4 nM or 1 nM) similar to or the same as ssRNAi 8 (IC₅₀ of 1 nM) using mouse primary hepatocytes were selected for further testing in mice. They were shown to be capable of causing about a 55% knockdown of the target in mouse liver at either a total dose of 300 mg/kg (ssRNAi 24) or a total dose of 100 mg/kg ssRNAi 27). The mice were sacrificed 48 hours after the last treatment.

In a subsequent experiment based on ssRNAi 27, the duration of suppression of PTEN mRNA in mouse liver was determined to be about 60%, 30% and negligible respectively at 24 hours, day 10 and day 30 after the last treatment using a 100 mg/kg total dose. In another experiment involving a 100 mg/kg total dose of ssRNAi 27, target levels at 48 hours after the last dose were determined to be 38% for kidney, 3% for skeletal muscle (quadricepts), 12% for lung and 5% for fat.

A dose response curve was generated for ssRNAi 27 using total doses of 50, 100, 200 or 300 mg/kg and measuring the PTEN mRNA in mouse liver 48 hours after the last dose. The respective percent reductions were approximately 25%, 50%, 65% and 65%.

ssRNAi 27 was also conjugated with the C16 carrier at nucleoside position 8 to produce ssRNAi 29 and used to treat mice with a 100 mg/kg total dose. Forty-eight hours after the last dose the PTEN mRNA levels in liver, kidney, skeletal muscle (quadricepts), lung and fat were determined and found to be approximately 50%, 25%, 20%, 30% and 35% respectively. Thus compared to ssRNAi 27, ssRNAi 29 was more active in skeletal muscle, lung and fat but less active in kidney while having about the same activity in liver.

Following the treatment of mice with either ssRNAi 24 or ssRNAi 27, it was demonstrated using the 5′RACE assay that liver PTEN mRNA was cleaved at the expected site for an AGO-2 catalytic effect.

The Lima et al. (2012) data show that their ssRNAi compounds have modest activity in liver and poor, or no activity in other organs/tissues, poor potency and short duration of effect. Since they did not use a carrier, or used a carrier incapable of sufficiently promoting the uptake of an siRNA, drug comparisons between their ssRNAi compounds and a cognate siRNA were not possible. siRNA compounds delivered to the liver by carrier, however, as well as antisense oligo drugs typically have substantially better activity, potency and duration of effect in liver compared to the Lima et al. ssRNAi compounds. Indeed, these investigators disclosed in a recent paper that ssRNAi compounds, including theirs, require further development in order to become commercially viable as drugs (Prakash et al., Nucleic Acids Res 43: 2993-3011, 2015).

### SUMMARY OF THE INVENTION

In accordance with the present invention, methods, modifications and ssRNAi compositions capable of providing RNAi activity in a wide range of organs/tissues/cell types against targets of interest in vitro and in vivo are disclosed. The novel ssRNAi compounds of the present invention are referred to herein as Accommodating Guide Strand RNAi (agsRNAi). Methods for producing agsRNAi rely on accommodating guide strand designs (AGSD) and the modifications involved are referred to as AGSD modifications.

Three subtypes of agsRNAi fall within the scope of the present invention: ags-siRNA compounds having activity against selected mRNA or regulatory RNA target(s) excluding miRNA; ags-IMiRs having activity against selected miRNA or any selected simtron or mirtron targets; and ags-MiRs mimics of selected endogenous miRNA or that have a novel targeting code sequence which is capable of modulating expression and/or function of a novel set of targets by an miRNA mechanism. In the latter case, the set of modulated targets are not engaged by the targeting code(s) present in naturally occurring miRNAs. Ags-siRNA, ags-IMiR and ags-MiR activities can be based on a catalytic or steric hindrance effect.

Ags-MiRs that use the seed region as their targeting code can be further subdivided into those having a sequence that is closely or entirely based on the sequence of the guide strand of the miRNA being mimicked (exclusive of the overhang) and those based on a modular design approach. Ags-MiRs based on the modular approach have the same sequence as the mimicked miRNA in positions 2-8 or 2-9 (so the seed sequence is retained) while the sequence ranging over positions 9 or 10 through 19 is not the same as the mimicked endogenous miRNA.

Ags-siRNA non-coding regulatory RNA targets include but are not limited to lncRNA, promoter associated RNA, enhancer RNA, snoRNA, piRNA, xiRNA, sdRNA, moRNA, MSY-RNA, tel-sRNA, crasiRNA and endogenous antisense RNA. Some ags-siRNA compounds can produce a change in expression and/or modulate the function of genes by directly engaging DNA targets on the basis of complementary base pairing including but not limited to one or more entities controlling particular gene(s) and selected from the group of promoters, enhancers or suppressors.

The fundamental problem with existing ssRNAi compounds is that the quality of their engagement with RISC, and argonaute protein in particular, is very substantially compromised by the lack of a passenger strand. The end result being reduced activity, potency and duration of effect on the target(s) compared to the cognate siRNA or miRNA.

AgsRNAi compounds are surprisingly superior to the ssRNAi compounds known in the art in one or more ways which include, without limitation, (1) Providing for a higher level of activity against the target(s) in vitro and/or in subjects; (2) Exhibiting a greater potency against the target(s) in vitro and/or in subjects; (3) Producing a significantly longer period of RNAi activity against the intended target(s) in vitro and/or in subjects; (4) More efficiently modulating the expression and/or function of targets in low oligo uptake tissues/cell types in subjects without the use of a carrier; (5) Inclusion of a variety of modifications that provide features such as enhanced nuclease resistance, enhanced plasma protein binding, enhanced effect on the intended target(s) and/or reduced off-target effects that can be made to ssRNAi compounds without undermining the RNAi activity; and (6) Increasing the activity, potency and/or duration of activity obtained in the absence of a 5′-phosphate or phosphate analog is not used.

Similar to conventional (double strand) siRNA and miRNA, the initial engagement of ss-RNAi and agsRNAi with the RNAi mechanism is considered as a two-step process. The first step involves a reversible association of the guide strand (in single strand form or in a duplex with a passenger strand) with the RISC loading complex (RLC). This step is not necessarily essential but can increase the efficiency of the second step. Dicer is a key component of the RLC. Dicer binds both single and double strand RNA primarily through interactions between its PAZ domain and the 3′end half of the guide strand Some single strand RNA binds more tightly to Dicer than a cognate double strand compound. It is believed that as part of the RLC, Dicer may, at least sometimes, play an important role in the loading of the guide strand of an RNAi trigger into the argonaute protein of RISC. This view is consistent with the differential guide strand regional binding preferences of Dicer (3′-end of the guide strand) and the argonaute (5′-end of the guide strand) proteins leaving aside any overhang or overhang precursor (Kini and Walton FEBS Lett 581: 5611-16, 2007; Lima et al., J Biol Chem 284: 2535-48, 2009).

Guide strands including agsRNAi compounds can be divided into regions based on the particular domains of the argonaute protein that they interact with at least on an intermittent basis (Ma et al., Nature 429: 318-322, 2004; Kini and Walton FEBS Lett 581: 5611-16, 2007; Lima et al., J Biol Chem 284: 26017-28, 2009; Schirle et al. Sci 346: 608-613, 2014). These interactions involve non-covalent charge-charge interactions between particular moieties in the guide strand and particular moieties in the associated domain of the argonaute protein. The relevant guide strand moieties include those associated with the sugar, sugar analog or sugar substitute (sugar) of a given nucleoside; those associated with the base associated with a given nucleoside; as well as those associated with the linkages between the nucleosides. These guide strand/argonaute regions of potential interaction are the following: (1) the 5′-end nucleosides in positions 1-2 are anchored to the MID domain. The 5′ end nucleoside is conjugated to a 5′-phosphate or phosphate analog, if any, as desired. The phosphate or phosphate analog has multiple attractive interactions with moieties in the MIDI domain. This 5′-end nucleoside is reoriented by conformational changes due to its interactions with the MIDI domain in a manner that makes it inaccessible to the target; (2) the nucleosides in positions 3-13 are in close proximity to the PIWI domain and the linkages between the nucleosides in positions 3-6 interact with moieties in this domain to stabilize the association while the nucleosides in positions 6 and 7 have more complex stabilizing interactions with the PIWI domain; (3) nucleosides that include positions 14-18 thread their way through a narrow channel between the PAZ and N domains. Positions 14-18 and positions 14 and 16 in particular are more sterically restricted by these domains in a manner that places greater constraints on the moieties projecting from these nucleosides such as any moiety in the 2′ sugar position; and (4) nucleoside 19 plus any other nucleosides and/or other structural units that complete the 3′-end of the guide strand interact with the PAZ domain. Any structural units in positions 20-23 in the case of agsRNAi are referred to as overhang precursors because they correspond to any overhang in a siRNA or miRNA guide strand.

Broadly speaking, therefore, AGSD methods and the AGSD modifications used to generate agsRNAi compounds can be more conveniently considered on the basis of subdividing the compounds into three major regions. Each of these regions includes both the nucleosides (or units when referencing the overhang precursor) and the intervening linkages, however, the nucleoside (or unit) position numbers are used to locate the regions as follows: (1) Nucleoside position 1; (2) Nucleoside positions 2-19; and (3) Any nucleosides or units in positions 20-23 that constitute any overhang precursor.

The superiority of agsRNAi over the ssRNAi known in the art relies, in part, on the modifications made to each of these three regions which collectively give rise to increases in both the affinity of the compound with the Dicer component of the RLC, and with the argonaute component of RISC as well as inreases the functional quality of the interaction with the argonaute protein. In addition, the modifications made to each of these regions must not undermine the functioning of the RNAi mechanism. This primarily means the modifications made to any intended agsRNAi cannot generate significant steric hindrance conflicts that interfere with the function of the RNAi mechanism, for example, by interfering with the conformational changes in the argonaute protein that must progressively occur as the target is engaged.

The degree to which each of these regions of an intended agsRNAi is optimized can affect the level of changes required in other regions, and indeed can reduce the degree to which the other regions need to be optimized in order to achieve the same level of activity, potency and/or duration of effect against the intended target(s). For example, it is known that a 5′-end phosphate substantially improves inhibitory function of ss-RNAi compounds known in the art. Notably, the presence of a 5′-end phosphate on the guide strand of an siRNA or miRNA has no significant effect on the functional abilities of these RNAi triggers. Thus, if the rest of the ssRNAi could be made as efficient in engaging and directing the RNAi mechanism as the guide strand that was delivered to the RNAi mechanism as a double strand RNAi trigger then the requirement for a 5′-phosphate on the ssRNAi would become irrelevant.

5′-end nucleoside modifications made to the nucleoside in position 1 used in agsRNAi compounds are those known to the skilled artisan or can be novel modifications provided by the present invention. The need for novel modifications at this position is particularly pressing for ssRNAi compounds that are to be administered to subjects without a protective carrier and where the compound benefits functionally from having a 5′ end phosphate or phosphate analog. Several modifications to this region of an ssRNAi provided by the present invention are alternatives, for example, to the 5′-VP-Tₘₒₑ modification in nucleoside position 1 known in the art. These novel modifications include those that involve the use of the natural 5′-phosphate along with modifications to the nucleoside in position 1 and possibly position 2 as well that substantially reduce the ability of phosphatases from removing this moiety while also providing for protection from 5′-3′-exoribonuclease attack. Alternative novel modifications provided by the present invention render the 5′-end nucleoside more susceptible to phosphorylation of its 5′ carbon by enzymes with this capability, such as hClp1 RNA kinase, while at the same time providing for increased 5′-3′-exoribonuclease resistance. The principal advantage of a number of these novel modifications is that by providing the means to have a 5′-end phosphate present when the agsRNAi engages with the RNAi mechanism, the functionality of the agsRNAi can be improved relative to the same compound with the 5′-VP-Tₘₒₑ modification known in the art. According to the present invention the 5′-VP moiety can also be conjugated certain nucleosides to produce novel nucleosides not known in the art that result in significantly superior function of the agsRNAi compared to an identical agsRNAi with the 5′-VP-Tₘₒₑ modification in nucleoside position 1.

The AGSD modifications made to nucleoside positions 2-19 of an agsRNAi compound increase the probability that the modified nucleoside will have its sugar in the C3′-endo conformation compared to the sugars (2′-fluoro, 2′-O-methyl and ribose) found in these positions in the ssRNAi compounds known in the art. AGSD modifications include certain nucleoside sugars as well as certain nucleoside bases. The AGSD modifications made to particular nucleosides also have the capacity to increase the probability that the nucleosides immediately adjacent to the modified nucleoside will also have their sugar in the C3′-endo confirmation.

The sugars provided herein have variable degrees of flexibility that reflect the ease with which they can undergo a conformational change away from the C3′-endo conformation. Sugars used in this region of agsRNAi compounds that are not AGSD modifications (ribose, 2′-fluro and 2′-O-methyl) are the most flexible. They are, therefore, the most susceptible to outside influences on their conformation. AGSD sugar modifications are all less flexible than these non-AGSD sugars but can be sub-categorized as being flexible, semi-flexible or rigid (Table 1). The less flexible they are the less susceptible they are to outside influences changing their conformation away from the preferred C3′-endo conformation. A modification that would negatively impact the expression of the 3′ endo conformation by contiguous nucleosides with more flexible sugars is the presence of one or more 2′-deoxyribonucleosides in the agsRNAi strand. For this reason 2′-deoxyribonucleosides are generally not used in positions 2-19 of agsRNAi compounds. 2′-deoxyribonucleosides, however, can be used in linkage sites in an agsRNAi where the linkage joining them is a boranophosphate.

The rigid AGSD sugars are held in a C3′-endo confirmation by a short chemical bridge inserted between the 2′ hydroxyl and 4 carbon positions of a ribose. Consequently, their conformation cannot be affected by outside influences, however, they are the most potent of the AGSD sugars in terms of their ability to affect the sugar conformations of contiguous nucleosides. The semi-flexible AGSD sugars have a longer chemical bridge joining two points in the sugar while the flexible AGSD sugars do not have such chemical bridges.

AGSD modifications also include certain nucleoside bases that substitute for U or C that can increase the likelihood that the nucleoside containing them and contiguous nucleosides will have their sugar in the C3′-endo confirmation, but the extent of this effect depends on the level of flexibility of the sugar found in the nucleoside in question. Again contiguous nucleosides with the most flexible sugars (ribose, 2′-fluro or 2′-O-methyl) will experience the greatest level of influence.

Nucleosides with purine bases (G or A) and a more flexible sugar are less likely to be in the C3′-endo confirmation compared to a nucleoside with a purine (C or U) base with the same more flexible sugar. The ability of an AGSD modification to a given nucleoside to increase the likelihood that the sugar in a contiguous nucleoside will be in the C3′-endo confirmation is affected by whether the contiguous nucleoside has a purine base or a purine base as well as by the level of flexibility of its sugar.

Rules are provided herein that establish the minimum AGSD modifications for inclusion in any given agsRNAi sequence that will result in a significant improvement in activity, potency and/or duration of effect on the intended target(s). Other rules establish the maximum proximity that AGSD sugars can have with each other. Too many such modifications, particularly when they are in too close a proximity, can result in suboptimal function for the agsRNAi compound.

In addition, the specific AGSD modifications provided and their positioning avoid steric hindrance and other adverse interactions between the agsRNAi and the RNAi mechanism that would significantly interfere with the ability of the agsRNAi to out perform an ssRNAi lacking AGSD modifications in this region.

The general effect of the AGSD modifications to this region on the agsRNAi compound is to make its conformation significantly more like the conformation it would have if it were in a duplex with a complementary passenger strand. The net functional effects of these modifications is the production of agsRNAi compounds which demonstrate enhanced activity, potency and/or longer duration of effect on the agsRNAi target(s) over ssRNAi known in the art.

Certain overhang precursors, which occur in one or more of positions 20-23, are described by the present invention for use in agsRNAi compounds. These include those that are not found in association with ssRNAi known in the art, but that have been used in siRNA. Those previously not used with ssRNAi can provide a substantially greater benefit to ssRNAi compounds including agsRNAi than they can when present in a cognate siRNA or cognate miRNA in terms of significantly increased activity, potency and/or duration of effect on the target.

In addition to the AGSD methods and AGSD modifications the current invention provides certain other improved capabilities for agsRNAi compounds that are of a more limited range. Increasing the binding affinity of the seed sequence for its targets, for example, is positively correlated with the activity the seed sequence of an agsRNAi compound against some or all of its targets. In the case of ags-MiRs increasing the activity could be a good outcome but for ags-siRNA and ags-IMiRs reducing the activity of the seed sequence by reducing its affinity for its targets is more likely to be a positive outcome since it could reduce unintended off-target effects.

AGSD modification(s) to the seed sequence typically will increase the binding affinity to its target(s), accordingly alterations in affinity must be assessed when comparing the activity of an ags-MiR to an ssRNAi with the same sequence. To make the comparison more accurate, the modifications to the seed sequence in both the ags-MiR and the ssRNAi mimic must be the same in both compounds. In this way any differences in activity, potency and/or duration of effect on the target(s) between these two compounds must be due to differences or modifications which occur outside of the seed sequence.

The conclusion that the level of the affinity of a seed sequence for its targets is generally positively correlated with the level of suppression of its targets can be inferred from the results obtained by Ui-Tei's group (Ui-Tei et al., Nucleic Acids Res 30: 7100-9, 2008) and later further expanded by them (Hibio et al., Scientific Rep 2:996 pages 1-10, 2012). Based on the Ui-Tei et al. (2008) data it is clear that affinity between a seed sequence and its mRNA target sequence is preferably above 21.5 degrees centigrade for Tm and/or below a AG of −12 for those mRNAs that are to be silenced and preferably below 15 degrees Tm and above −11 AG for those that are not to be silenced. These figures set a useful standard of designing ags-MiR compounds with enhanced activity against their targets and for designing ags-siRNA and ags-IMiR compounds with reduced off target effects due to the seed sequence.

Other nucleoside modifications are provided herein including 2,6-diaminopurine (used in place of adenine) that can boost the binding affinity of the seed sequence for its targets (Table 2). This modification can also prevent the adenosine to inosine editing in ags-MiR compounds that can sometimes occur to adenosines in the seed region of naturally occurring miRNAs. The effect of such editing has been shown to substantially alter the mRNA target profile of the altered miRNA (Kume et al., Nucleic Acids Res 42: 10050-60, 2014).

The duration of the effect of an agsRNAi on its target(s) in cells in the body, in part, is a function of how stable it is in the cells. Hence, methods that reduce the rate of turnover can be used to increase the duration of its effect. The clearance of single strand therapeutic oligos from cells in the body follows cleavage of the oligo into two or more parts by intracellular nucleases, most often by endonucleases. Once cleaved the oligo fragments escape from the cell and most of the fragments end up in the urine.

There are important species and organ/tissue/cell type differences in the rate of oligo clearance from particular cells in the body due to differences in the rate of cleavage for the same oligo. Cleavage rate differences can be quite large and are typically measured in days or weeks (greater than 5 fold difference in some comparisons). For example, in general the livers of numerous species of subjects cleave these oligos more rapidly than many other organ/tissue/cell types in the same species. As a further example, the organs, tissues and cells in mice typically are more active in cleaving oligos than the corresponding organs, tissues and cells in other species used in drug development including rats and monkeys (Geary et al., Drug Metab Disposition 31: 1419-28, 2003).

Similarly, the rate of clearance of agsRNAi compounds from a subject is both species and organ/tissue/cell type dependent. Thus, it is important to consider in which organ/tissue/cell type(s) and in what species is it desirable to have a prolonged effect on the target(s) relative to other organ/tissue/cell types in the subject. AgsRNAi cleavage in cells can be quantified and the specific linkage sites and the rate at which they are cleaved can be determined using liquid-chromatography-coupled mass spectroscopy (Lima et al., Cell 150: 883-94, 2012).

When an agsRNAi is degraded in a particular organ/tissue/cell type more quickly than is desired relative to other organ/tissue/cell types in the same subject, there are at least two possible solutions: (1) the offending cleavage sites and the rate of cleavage of the agsRNAi can be determined for particular organ/tissue/cell types. Using the guidance provided herein, it is possible to increase the nuclease resistance of the cleaved site(s) without unduly affecting the desired level of RNAi activity; and (2) an appropriate passenger strand complementary to the agsRNAi can be administered to the subject.

An appropriate passenger strand is one that protects the agsRNAi not engaged by RISC from intracellular nuclease attack until the agsRNAi is loaded into an argonaute protein. In addition, an appropriate passenger strand is one that is designed to facilitate the loading of the guide strand, in this case an agsRNAi, into RISC in accordance with the principles known in the art, for example: (1) the thermodynamic asymmetry rule that involves the terminal 4 nucleosides in each strand that are duplexed with the other strand must be applied. Specifically, the duplexed end that includes the 5′-end of the passenger strand must have a higher binding affinity with the agsRNAi strand compared to the opposite duplexed end that includes the 5′-end of the agsRNAi (Khvorova et al., Cell 115: 209-16, 2003; Schwarz Cell 115: 199-208, 2003); and (2) for agsRNAi in general the central region of the passenger strand comprising nucleosides 8-11 counting from the 5-end preferably has a nucleoside selected from the group consisting of a single mismatch with the agsRNAi, an abasic nucleoside, an UNA containing nucleoside, a nucleoside with a 2,4 difluorotoyl base and a nucleoside with a 5-nitroindole base (Liu et al., Science 305: 1437-41, 2004; Meister et al., Mol Cell 15: 185-97, 2004; Yoda et al., Nature Struct Mol Biol 17: 17-23, 2010; WO 2010/011895). For ags-siRNA and ags-IMiRs, however, the passenger strand linkage site defined by nucleosides in positions 9 and 10 (opposite nucleoside positions 10 and 11 in the ags-siRNA or ags-IMIR) optionally has a phosphodiester linkage and these nucleosides independently have a ribose and/or 2′-fluoro sugar.

If the passenger strand is not delivered to cells in a subject by means of a protective carrier then it must be sufficiently modified to resist degradation by extracellular nucleases between the time it is administered and the time that it enters cells. Further, such modifications cannot undermine the intended RNAi activity. Consequently, it is preferable that the passenger strand is designed as a seqRNAi passenger strand in accordance with the methods and compositions disclosed in WO 2012/145729). Alternatively, the passenger strand is designed using compositions and other methods known in the art for producing a passenger strand, for example those found in the McSwiggen patents and filings that cover a range of possible passenger strand modifications and modification placements in the strand (WO 03/070918; WO 03/074654; WO 2005/019453; and WO 2007/022369 along with the related filings and patents). The McSwiggen passenger strands, however, have not been designed for single strand administration to a subject without a protective carrier. As a result, a protective carrier and/or added nuclease resistant linkages added using one of the patterns provided for herein.

A carrier could be used to target the passenger strand to the specific organ/tissue/cell type(s) where the agsRNAi is to be protected. For example, an enveloping nanoparticle carrier known in the art, such as the Tekmira SNALP carrier (WO 2009/086558; WO 2010/042877), or one the carriers that incorporate a ligand(s) for the asialoglycoprotein receptor (such as ones based on N-acetylglucosamine) could be conjugated to the passenger strand (Rozema et al., Proc Natl Acad Sci (USA) 104: 12982-87, 2007; Nair et al. J Am Chem Soc 136: 16958-61, 2014; WO 2008/131419; WO 2011/104169). These carrier examples are known to efficiently deliver oligo drugs to the liver in a number of species including humans.

One or more of the following sets of conditions can document various aspects of the surprisingly better performance of a particular agsRNAi of the present invention over a corresponding ssRNAi designed using methods and modifications known in the art:

A single strand modified oligoribonucleotide agsRNAi composition (ags-siRNA, ags-IMiR or ags-MiR) for modulating the expression and/or function of at least one target nucleic acid sequence expressed by subject cells;
wherein said subject cells are of the same type and are selected from the group consisting of a particular cell line; a representative sampling of a particular organ, gland or neoplastic growth; an enriched sample of parenchymal cells from a particular organ, gland or neoplastic growth; a particular type of epithelial tissue including simple, stratified, pseudostratified and transitional; a particular type of connective tissue where the general types are loose and dense forms as well as more specialized types that include reticular, adipose, blood and lymphoid connective tissues; a particular type of nervous tissue subdivided by location (such as brain, spinal cord, ganglion, and nerve) and/or by cell type, i.e., neuronal or glial; and a particular type of muscle tissue where the types are skeletal, cardiac and smooth; and wherein said composition comprises:
I) A nucleoside in position 1 and an associated linkage with the nucleoside in position 2 where the modifications to these structure are provided in the section entitled “Designing AgsRNAi Compounds: 5′-End Modifications”; and
II) 19 nucleosides
   a) where the modifications in positions 2-19 provide for basic nuclease resistance where the various sets of basic nuclease resistance designs are provided in the section entitled “Designing AgsRNAi Compounds: Providing Basic Nuclease Resistance”; and
   b) where there are at least 4 AGSD modifications where AGSD and AGSD modifications are provided in the section entitled “Designing AgsRNAi Compounds: Nucleoside Positions 2-19”; and
III) optionally has 1-4 overhang precursor units where the design of overhang precursors are provided in the section entitled “Designing AgsRNAi Compounds: Overhang Precursors”; and
IV) a region of complementary base pairing with the target nucleic acid(s) (referred to as the targeting code) that
   a) for an ags-MiR is either 6 or 7 consecutive nucleotides in length beginning at position 2 or is 11 or 12 consecutive nucleosides in length beginning at position 4 or 5 although optionally position 8 or 9 can have a mismatch with the target; or
   b) for an ags-siRNA or ags-IMiR is at least 15 consecutive nucleosides in length beginning at position 2 and extending through position 16 although optionally position 8 or 9 can have a mismatch with the target; and

wherein
said modified oligoribonucleotide agsRNAi strand meets one or more of the following sets of criteria (A-J) for demonstrating at least one of the advantages of an agsRNAi compound over ssRNAi compounds known in the art; and:
A) the agsRNAi composition is an ags-siRNA or ags-IMiR and provides a maximal plateau level of activity in a dose response curve against the target in said cells that constitutes at least a 50, 60, 70, 80 or 90% change in expression and/or function of the target and said level of change in activity is equal to or greater than 20, 30 or 40 percentage points greater (e.g., for greater than 20 percentage points the ssRNAi activity would be less than 30, less than 40, less than 50, less than 60 or less than 70% respectively) than the maximal level of activity obtained using an ssRNAi of the same sequence, same 5′-phosphate or 5′-phosphate analog, if any, and lacking the AGSD modifications found in the agsRNAi as well as administered using the same dosage regimen under one or more of the following conditions of administration of the two compounds to be compared:
   a) to a mammalian cell line or primary mammalian parenchymal cells grown in vitro where the compounds to be compared are delivered to said cells using a method selected from the group transfection, electroporation and gymnosis; or
   b) to a subject where the compounds to be compared are delivered to said cells with an enveloping protective carrier; or
   c) to a subject where the compounds to be compared are delivered to said cells without an enveloping protective carrier; or
B) the agsRNAi composition is an ags-MiR and provides a maximal plateau level of activity in a dose response curve(s) against at least 1, 2, 3 or 4 targets in said cells where each maximal plateau level of activity constitutes at least a 20, 30, 50, 60, or 70% change in expression and/or function per target and said level of change in activity is equal to or greater than 20, 30 or 40 percentage points greater (e.g., for greater than 20 percentage points the ssRNAi activity would be 0 or less than 10, less than 30, less than 40 or less than 50% respectively) than the maximal level of activity obtained using an ssRNAi with the following properties in common with the ags-MiR: i) the same sequence; (2) the same 5′-phosphate or 5′-phosphate analog, if any; and (3) the same modifications in the seed sequence.
wherein both the ags-MiR and the ssRNAi are administered using the same dosage regimen under one or more of the following conditions:
   a) to a mammalian cell line or primary mammalian parenchymal cells grown in vitro where the compounds to be compared are delivered to said cells using a method selected from the group transfection, electroporation and gymnosis; or
   b) to a subject where the compounds to be compared are delivered to said cells with an enveloping protective carrier; or
   c) to a subject where the compounds to be compared are delivered to said cells without an enveloping protective carrier; or
C) the agsRNAi composition is an ags-siRNA or ags-IMiR that provides an IC₅₀ or EC₅₀ against the target in said cells where the amount of the agsRNAi compound is at least 2, 4, 8, 16 or 32 fold lower than the amount of an ssRNAi of the same sequence, same 5′-phosphate or 5′-phosphate analog, if any, but lacking AGSD modifications as well as administered using the same dosage regimen needed to achieve an IC₅₀ or EC₅₀ against the target in said cells under one or more of the following conditions of administration of the two compounds to be compared:
   a) to a mammalian cell line or primary mammalian parenchymal cells grown in vitro where the compounds to be compared are delivered to said cells using a method selected from the group transfection, electroporation and gymnosis and the IC₅₀ or EC₅₀ is measured as a molarity; or
   b) to a subject where the compounds to be compared are delivered to said cells with an enveloping protective carrier and the IC₅₀ or EC₅₀ is measured as mg/kg administered to the subject; or
   c) to a subject where the compounds to be compared are delivered to said cells without an enveloping protective carrier and the IC₅₀ or EC₅₀ is measured as mg/kg administered to the subject; or
D) the agsRNAi composition is an ags-MiR that provides an IC₅₀ or EC₅₀ against at least 1, 2, 3 or 4 target(s) in said cells where the amount of the agsRNAi compound is at least 2, 4, 8, 16 or 32 fold lower than the amount of an ssRNAi with the following properties in common with the ags-MiR: i) the same sequence; (2) the same 5′-phosphate or 5′-phosphate analog, if any; and (3) the same modifications in the seed sequence. The ssRNAi, however, lacks any other AGSD modifications present in the ags-MiR.
wherein both the ags-MiR and the ssRNAi are administered using the same dosage regimen needed to achieve an IC₅₀ or EC₅₀ against the target in said cells under one or more of the following conditions of administration of the two compounds to be compared:
   a) to a mammalian cell line or primary mammalian parenchymal cells grown in vitro where the compounds to be compared are delivered to said cells using a method selected from the group transfection, electroporation and gymnosis and the IC₅₀ or EC₅₀ is measured as a molarity; or
   b) to a subject where the compounds to be compared are delivered to said cells with an enveloping protective carrier and the IC₅₀ or EC₅₀ is measured as mg/kg administered to the subject; or
   c) to a subject where the compounds to be compared are delivered to said cells without an enveloping protective carrier and the IC₅₀ or EC₅₀ is measured as mg/kg administered to the subject; or
E) the agsRNAi composition is an ags-siRNA or ags-IMiR and provides at least a 50, 60, 70, 80 or 90% change in expression and/or function of the target in said cells at a time point after the last treatment where at that time point the level of change in expression and/or function of the target in said cells is equal to or greater than 40, 50, 60, 70 or 80 percentage points higher (e.g., for greater than 50 percentage points the ssRNAi activity would be 0, 0, less than 10, less than 20 or less than 30% respectively) than the level obtained at that time point using an ssRNAi of the same sequence, same 5′-phosphate or 5′-phosphate analog, if any, and lacking the AGSD modifications as well as administered using the same dosage regimen under one or more of the following conditions of administration of the two compounds to be compared:
   a) to a mammalian cell line or primary mammalian parenchymal cells grown in vitro where the compounds to be compared are delivered to said cells using a method selected from the group transfection, electroporation and gymnosis; or
   b) to a subject where the compounds to be compared are delivered to said cells with an enveloping protective carrier; or
   c) to a subject where the compounds to be compared are delivered to said cells without an enveloping protective carrier; or
F) the agsRNAi composition is an ags-MiR and provides at least a 20, 30, 50, 60, or 70% change in expression and/or function per target for at least 1, 2, 3 or 4 targets in said cells at a time point after the last treatment where the level of change in expression and/or function of the target in said cells is equal to or greater than 20, 30, 40, 50, 60 or 70 percentage points higher (e.g., for greater than 30 percentage points the ssRNAi activity would be 0, 0, less than 20, less than 30 or less than 40% respectively) than the level obtained at that time point using an ssRNAi with the following properties in common with the ags-MiR: i) the same sequence; (2) the same 5′-phosphate or 5′-phosphate analog, if any; and (3) the same modifications in the seed sequence. The ssRNAi, however, lacks any other AGSD modifications present in the ags-MiR.
wherein both the ags-MiR and the ssRNAi are administered using the same dosage regimen under one or more of the following conditions:
   a) to a mammalian cell line or primary mammalian parenchymal cells grown in vitro where the compounds to be compared are delivered to said cells using a method selected from the group transfection, electroporation and gymnosis; or
   b) to a subject where the compounds to be compared are delivered to said cells with an enveloping protective carrier; or
   c) to a subject where the compounds to be compared are delivered to said cells without an enveloping protective carrier; or
G) the agsRNAi composition is an ags-siRNA or ags-IMiR and is systemically administered to a subject without an enveloping protective carrier that increases the amount of an oligo taken up by said cells
wherein
   a) the agsRNAi provides at least a 50, 60, 70, 80 or 90% change in expression and/or function of the target in said cells and said cells are not a liver organ sample or an enriched hepatocyte sample; and
   b) the ratio of the weight in grams of the intact agsRNAi in said cells divided by a unit weight of said cells expressed in grams compared to the weight in grams of the intact agsRNAi in said sample of the liver organ or enriched hepatocyte population from the liver organ sample divided by a unit weight of sample of the liver organ or enriched hepatocyte population from the liver organ sample expressed in grams is less than 0.3, less than 0.2, or less than 0.1 (e.g., if the agsRNAi weight per gram of lung sample is 43±10 and the agsRNAi weight per gram of liver sample is 950±129 then the ratio of lung to liver sample is 0.05 which is less than 0.1); and
wherein under the same conditions the level of change in expression and/or function of the target in said cells treated with said agsRNAi is equal to or greater than 40, 50, 60, 70 or 80 percentage points higher (e.g., for greater than 50 percentage points the ssRNAi activity would be 0, less than 10, less than 20, less than 30, less than 40% respectively) than the level obtained using an ssRNAi of the same sequence, same 5′-phosphate or 5′-phosphate analog, if any, and lacking AGSD modifications without an enveloping protective carrier that increases the amount of an oligo taken up by said cells; or
H) the agsRNAi composition is an ags-MiR and is systemically administered to a subject without an enveloping protective carrier that increases the amount of an oligo taken up by said cells
wherein
   a) the ags-MiR provides at least a 20, 30, 50, 60, or 70% change in expression and/or function against at least 1, 2, 3 or 4 targets in said cells and said cells are not a liver organ sample or an enriched hepatocyte sample; and
   b) the ratio of the weight in grams of the intact ags-MiR in said cells divided by a unit weight of said cells expressed in grams compared to the weight in grams of the intact ags-MiR in said sample of the liver organ or enriched hepatocyte population from the liver organ sample divided by a unit weight of sample of the liver organ or enriched hepatocyte population from the liver organ sample expressed in grams is less than 0.3, less than 0.2, or less than 0.1 (e.g., if the ags-MiR weight per gram of lung sample is 43±10 and the ags-MiR weight per gram of liver sample is 950±129 then the ratio of lung to liver sample is 0.05 which is less than 0.1); and
wherein
under the same conditions the level of change in expression and/or function of the target in said cells treated with ags-MiR is equal to or greater than 20, 30, or 40 percentage points higher (e.g., for greater than 20 percentage points the ssRNAi activity would be 0, less than 10, less than 20, or 40% respectively) than the level obtained using an ssRNAi with the following properties in common with the ags-MiR: i) the same sequence; (2) the same 5′-phosphate or 5′-phosphate analog, if any; and (3) the same modifications in the seed sequence. The ssRNAi, however, lacks any other AGSD modifications present in the ags-MiR and administered without an enveloping protective carrier that increases the amount of an oligo taken up by said cells; or
I) A kit comprising a first composition that is an agsRNAi where it is an ags-siRNA or an ags-IMiR and a second composition comprising a passenger strand that is complementary to the first composition wherein the compositions are separately administered to said cells such that the first or second composition is taken up by the cells prior to the administration of the other composition to the cells;
   wherein
   a) a duplex of first and second compositions functions as an siRNA
   b) the passenger strand promotes the loading of the agsRNAi into RISC in preferences to itself; and
   c) the first and second compositions are administered to cells grow in vitro or in a subject according to one of the following sets of circumstances:
      i) mammalian cells grown in vitro where mammalian cells are selected from the group consisting of a cell line, primary cells and primary parenchymal cells and where both compounds are delivered to said cells using a method selected from the group transfection, electroporation and gymnosis; or
      ii) a subject where both of the compounds are delivered to said cells with an enveloping protective carrier; or
      iii) a subject where one of the compounds is delivered to said cells with an enveloping protective carrier; or
      iv) a subject where the both compounds are delivered to said cells with a carrier other than an enveloping protective carrier and the passenger strand is modified to be sufficiently nuclease resistant for systemic administration to the subject; or
      v) a subject where the one compounds is delivered to said cells with a carrier other than an enveloping protective carrier; or
      vi) a subject where neither of the compounds are delivered to said cells by a carrier; and
wherein
at a certain time point after the last treatment the level of change in expression and/or function of the target in said cells is equal to or greater than 40, 50, 60, 70 or 80 percentage points higher than the level obtained at that time point using the agsRNAi in the absence of the passenger strand; or
J) the agsRNAi is an ags-MiR and provides at least a 20, 30, 50, 60, or 70% change in expression and/or function per target for at least 1, 2, 3 or 4 targets in said cells at a time point after the last treatment where the level of change in expression and/or function of the target in said cells is equal to or greater than 20, 30, 40, 50, 60 or 70 percentage points higher (e.g., for greater than 30 percentage points the ssRNAi activity would be 0, 0, less than 20, less than 30 or less than 40% respectively) than the level obtained at that time point using an ssRNAi with the following properties in common with the ags-MiR: i) the same sequence; (2) the same 5′-phosphate or 5′-phosphate analog, if any; and (3) the same modifications in the seed sequence. The ssRNAi, however, lacks any other AGSD modifications present in the ags-MiR.
wherein both the ags-MiR and the ssRNAi are administered using the same dosage regimen under one or more of the following conditions:
   a) to a mammalian cell line or primary mammalian parenchymal cells grown in vitro where the compounds to be compared are delivered to said cells using transfection; or
   b) to subjects where the compounds to be compared are delivered to said cells with an enveloping protective carrier; or
   c) to subjects where the compounds to be compared are delivered to said cells without an enveloping protective carrier.

The miRNA sequences and nomenclature used herein are taken from the miRBase (www.mirbase.org). The nomenclature has been described in Griffiths-Jones et al., Nucleic Acids Res 34: D140-D144, 2006. In brief, numbers that immediately follow the designation miR-, for example, miR-29, designate particular miRNAs. The specific designation is applied to the corresponding miRNAs across various species. Letters, for example in miR-34a and miR-34b, distinguish particular miRNAs differing in only one or two nucleoside positions in the mature miRNA (guide or antisense strand that loads into RISC). Numbers following a second dash, for example in miR-24-1 and miR-24-2, distinguish distinct loci that give rise to identical mature miRNAs. Multiple miRNAs family members that differ in only one or two nucleoside positions in the mature mRNA from some other family member(s) and come from distinct hairpin loci have both letters and additional numbers following the letters, for example, miR-29b-1 and miR-29b-2. Finally, in some instances two different mature miRNA sequences are excised from the same hairpin precursor where one comes from the 5′ arm and the other from the 3′ arm. These are designated -5p and -3p respectively, for example, miR-17-5p and miR-17-3p

### BRIEF DESCRIPTION OF THE DRAWINGS

FIG. 1: CENA

FIG. 2: HM

FIG. 3: UNA (Unlocked Nucleic Acid)

FIG. 4: ANA

FIG. 5: EA

FIG. 6: 6A: HNA; 6B: 3′-FHNA

FIG. 7: AENA

FIG. 8: CRN (Confomationally Restricted Nucleoside); 8A: R monomer; 8B: Q monomer

FIG. 9: LNA (Locked Nucleic Acid); 9A; classic LNA and α-L-LNA; 9B: Thio-LNA and Amino LNA

FIG. 10: Five Examples of Abasic Nucleosides

FIG. 11: CeNA

FIG. 12: 5′ Carbon: Site for 5′-End Terminal Modifications

FIG. 13A: 2′-O-Methoxyethyl (2′-MOE) Modification of a Ribonucleoside; 13B: Ethyl Bicyclic Nucleic Acid (cEt)

FIG. 14: C10 (TC10) and C16 (TC16) Conjugation to a Nucleoside Illustrated with a Thymine Containing Nucleoside

FIG. 15: Structure for 5′-(E)-vinyl-phosphonate Conjugated to a Thymine Containing 2′-O-MOE Nucleoside (5′-VP-T)\

FIGS. 16A-E. Linkages useful in the compounds of the invention. FIG. 16A: phosphodiester. FIG. 16B: phosphorothioate. FIG. 16C: N3 phosphoramidate; FIG. 16D: amide linkage. FIG. 16E: boranophosphate linkage.

FIG. 17: Synthesis of 3′-H-Boranophosphonate Monomers 3a-e. summarizes the synthesis of the 2′-deoxyribonucleoside 3′-H-boranophosphonate monomers 3a-d and locked nucleic acid (LNA) thymidine 3′-H-boranophosphonate monomer 3e. 2′-Deoxythymidine monomer 3a was obtained in 95% from the thymidine derivative bearing the 3′-OH 1a and pyridinium H-boranophosphonate 2. 2′-Deoxyadenosine, cytosine, guanosine, and LNA thymidine monomers 3b-e were synthesized by the method for the synthesis of 3a with some modifications.

FIG. 18: Solid-Phase Synthesis of PBX-ODNs. Scheme 2 shows the synthesis of P-boronated oligodeoxyribonucleotides bearing oxygen, sulfur, or 2-morpholinoethylamino as the substituent X on the phosphorus atoms (PBX-ODNs) via H-boranophosphonate oligodeoxyribonucleotides (PBH-ODNs). The monomers 3a-e were condensed with the 5′-OH of nucleosides or oligos on a controlled-pore glass (CPG) in the presence of 1,3-dimethyl-2-(3-nitro-1,2,4-triazol-1-yl)-2-pyrrolidin-1-yl-1,3,2-diazaphospholidinium hexafluorophosphate (MNTP)19 and 2,6-lutidine, and the 5′-end was deprotected by 3% dichloroacetic acid (DCA). Released dimethoxytrityl (DMTr) cations were reduced by Et3SiH, because they would otherwise cause side reactions with the internucleotidic BH3 groups. The PBH-ODN chains were elongated by this cycle, and the modification of the phosphorus atoms and subsequent deprotection afforded PBX-ODNs.

### DETAILED DESCRIPTION OF THE INVENTION

### A. Overview of Prior Art

Plasterk's group provided the first publication describing ssRNAi in January 2002 (Tijsterman et al., Science 295: 694-97, 2002). It appeared before the priority date for the first ssRNAi patent filing in July 2002 (WO 2004/007718) by Tuschl and his colleagues. The Plasterk ssRNAi compounds had a 5′-end phosphate group. Naturally occurring siRNA and miRNA have a 5′-end phosphate on each strand, but it has been shown that double strand RNAi drugs do not require this structure to be active. ssRNAi compounds known in the art when tested in tissue culture, however, typically have higher activity if they are manufactured with a 5′-end phosphate group or a suitable analog. The issued Tuschl ssRNAi patent (U.S. Pat. No. 8,101,348) based on the July 2002 PCT filing includes a number of different 5′-end phosphate analogs, but the method of use claim involving these analogs is restricted to silencing targets in tissue culture.

Plasterk's initial publication on ssRNAi and the initial patent filing (WO 2004/007718) on ssRNAi was soon followed by a number of other early publications and another patent filing (Martinez et al., Cell 110: 563, 2002; Schwarz et al., Mol Cell 10: 537, 2002; Holen et al., Nucleic Acids Res 31: 2401, 2003; WO 2004/063375).

The initial burst of activity focusing on ssRNAi that began in 2002 quickly subsided. Likely reasons include the generally poor tolerance of ssRNAi to chemical modifications that could impart sufficient nuclease resistance to permit its use in subjects, even if used with a protective carrier.

In the first of two outliers Hall et al., (2004) showed that the incorporation of boranophosphate linkages along with phosphodiester linkages into the guide strands of siRNA compounds could frequently substantially increase their potency. The main exception was the finding that using a 5′-(α-P-borano) triphosphate to insert a nucleoside in more than one of the 5 nucleosides in positions 8-12 counting from the 5′-end of the guide strand had an inhibitory effect on potency. The authors suggested this negative effect might be due to the fact that this region includes positions 10 and 11 that are opposite the target cleavage site for AGO-2. Making boranophosphate linkage modifications to the passenger strand had little effect on potency. These ssRNAi compounds were synthesized using a DNA template, 5′-(α-P-borano) triphosphates and T7 RNA polymerase. Given this approach the boranophosphate linkages necessarily were associated with nucleosides having one or more of a particular type of base. There were no other modifications in these ssRNAi compounds and no modifications in the cognate siRNA compounds used as a comparator. The boranophosphate linkage provided substantial added nuclease resistance for the sites in an RNA strand where they are found but the ssRNAi compounds studied were not sufficiently stabilized for use in subjects. RNase A treatment of the modified and unmodified ssRNAi strands were both degraded at the same rate.

The guide strand from the most potent siRNA in the Hall et al. (2004) was evaluated in the studies published in 2006 by this group. In both of these papers, the target was enhanced green fluorescence protein (EGFP) under the control of an inducible promoter and expressed in Hela cells. The RNAi triggers were transfected into the Hela cells. The ssRNAi and cognate siRNA compounds without boranophosphate linkages that were tested were 21-mers with UU overhangs.

Remarkably the boranophosphate modified ssRNAi was found to be about 8-fold more potent than the cognate unmodified siRNA, IC₅₀=about 1 nm vs. IC₅₀=about 8 nm respectively. The peak activity was also greater for the ssRNAi being about 99% target suppression vs. 70% respectively. The effect of the ssRNAi and the cognate siRNA were both shown to be due to an AGO-2 mediated cleavage of EGFP mRNA. All the nucleosides in this ssRNAi with cytosine (total of 6) or adenine (total of 2) were linked to the adjacent nucleoside by a boranophosphate linkage (total of 8 boranophosphate linkages). Other patterns of modification where different nucleoside base type were associated with the boranophosphate linkage also boosted activity of this ssRNAi relative to the cognate siRNA. This occurred as long as the pattern of boranophosphate modification did not cause the majority of the nucleosides in positions 8-12 to be linked to a contiguous nucleoside by a boranophosphate linkage. By evaluating a second ssRNAi sequence that had a different ratio of nucleosides defined by their bases it was shown that the adverse effect of the modified linkage being associated with a nucleoside in positions 8-12 was not dependent on the base but rather on the number of boranophosphate linkages in this region.

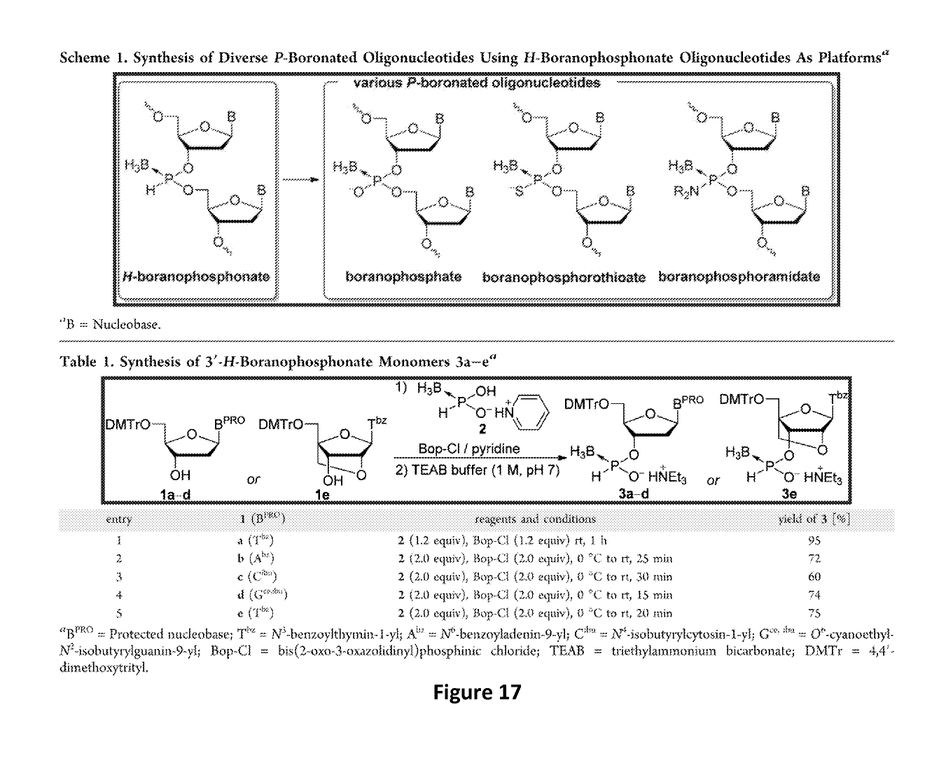

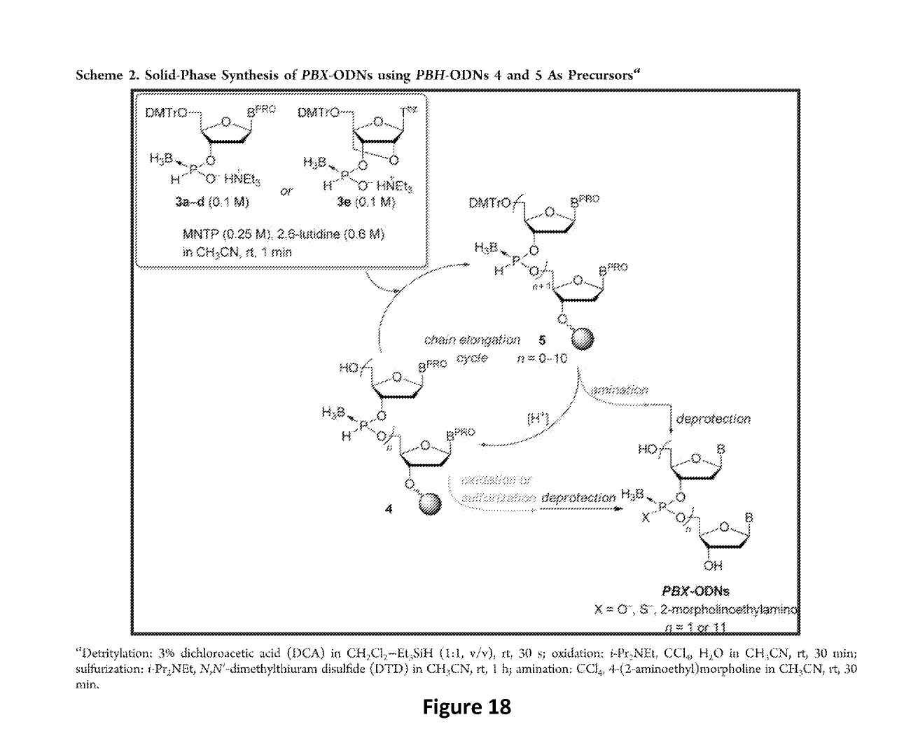

Taken together Hall et al. (2004 and 2006) show that boranophosphate mixed with phosphodiester linkages frequently can substantially boost the effectiveness of guide strands against their targets in both the siRNA and ssRNAi formats. These studies, however, to not provide the information necessary for determining the optimal placement of the boranophosphate linkages in these guide strands since it was not possible to dissociate this linkage from the base type of its associated nucleoside. The nucleoside positions associated with the boranophosphate linkage in the most active ssRNAi, however, were 4, 5, 6, 7, 11, 14, 15 and 19. More recently methods for chemically incorporating boranophosphate linkages into oligonucleotides have been developed. Synthetic schemes are provided in FIGS. 17 and 18. Also see Uehara et al., J Organic Chem 79: 3465-72, 2014.

As discussed in more detail in the “Background of the Invention,” in 2012, interest in ssRNAi was again stimulated. Two publications each from Isis Pharmaceuticals and Sirna Therapeutics (owned by Merck) demonstrated for the first time that ssRNAi could routinely be developed with the capacity to substantially inhibit messenger RNA targets of choice in the livers or brains of mice. One Sirna study also dealt with ssRNAi compounds with miRNA mimic activity. The three published patent applications associated with these publications have 2010 priority dates (WO 2011/139699, WO 2011/139702 and WO 2012/027206). Isis filed the first two of these and Merck filed the third. The single Merck patent filing based on the Sirna data (WO 2012/027206) disclosed in the two 2012 papers is limited to compounds comprising non-nucleic acid spacers, which is an optional feature of their ssRNAi compounds. These spacers are included to facilitate attachment if various structures such as fluorescent tags. The Merck Sirna filing (2010 priority date and published March 2012) did not disclose ssRNAi compounds with miRNA mimic activity.

Originally it was assumed that the 2′-fluoro modification, which is extensively used in the newer ssRNAi, promotes a higher binding affinity between it and a complementary RNA or DNA strand as a result of a proposed 2′-fluoro-dependent conformational preorganization that favors the A-type helical conformation when a duplex is formed. Recent detailed conformational studies, however, have shown that there is little difference between unmodified single stranded RNA in this regard and single stranded 2′-fluoro RNA (Pallan et al., Nucleic Acids Res 39: 3482, 2011). Instead the effects of this modification on duplex stability have been found to be due to enthalpy. Duplexes involving 2′-fluoro RNA strand or strands are less hydrated than unmodified RNA duplexes.

### B. Definitions and Defined Phrases

The following definitions and defined phrases are provided to facilitate an understanding of the invention.

“2′-fluoro” refers to a nucleoside modification where the fluorine has the same position and stereochemical orientation as the 2′-hydroxyl in β-D-ribofuranose. In instances where the fluorine has the same orientation as the 2′-hydroxyl in β-D-arabinofuranose, the associated nucleoside is referred to as FANA or 2′-deoxy-2′-fluoro-arabinonucleic acid.

“2′-O-methyl” refers to a 2′-O-methyl ribose modification.

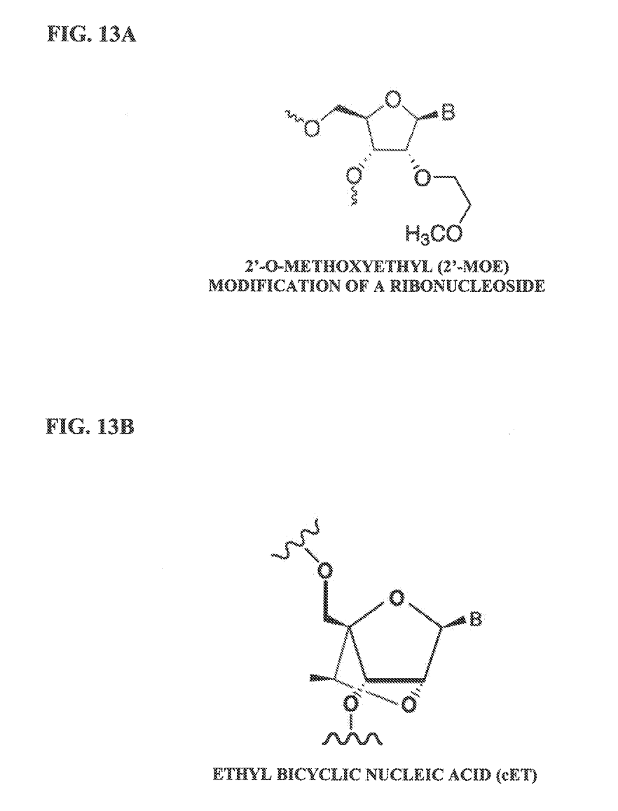

“2′-MOE” or “MOE” refers to a 2′-O-methyoxyethyl ribose modification as illustrated in FIG. 12A.

“3′-supplementary or 3′-compensatory sites” refers to sites in some miRNA guide strands down-stream of the seed sequence that are complementary to the target sequence and contribute to target selection particularly when the seed sequence has a weak match with the target.

“3′UTR” is an abbreviation for the 3′ untranslated region of an mRNA.

“4'S-FANA” refers to 2′-deoxy-2′-fluoro-4′-thioarabinonucleic acid (Watts et al., Nucleic Acids Res 35: 1441-51, 2007)

“5′-end module” refers to the 5′-end terminal 8 or 9 nucleosides found in an ags-MiR or ssRNAi with miRNA-like activity where the compound is constructed using the modular approach.

“5′-phosphate” or “5′-phosphate analog” refers to a phosphate or phosphate analog conjugated to the 5′ carbon of the 5′-end nucleoside sugar of an RNAi trigger.

“5′-to-3′ mRNA decay pathway” refers to a naturally occurring pathway for degrading mRNA that is initiated by the removal of the poly(A) tail by deadenylases. This is followed by removal of the 5′-cap and subsequent 5′ to 3′ degradation of the rest of the mRNA.

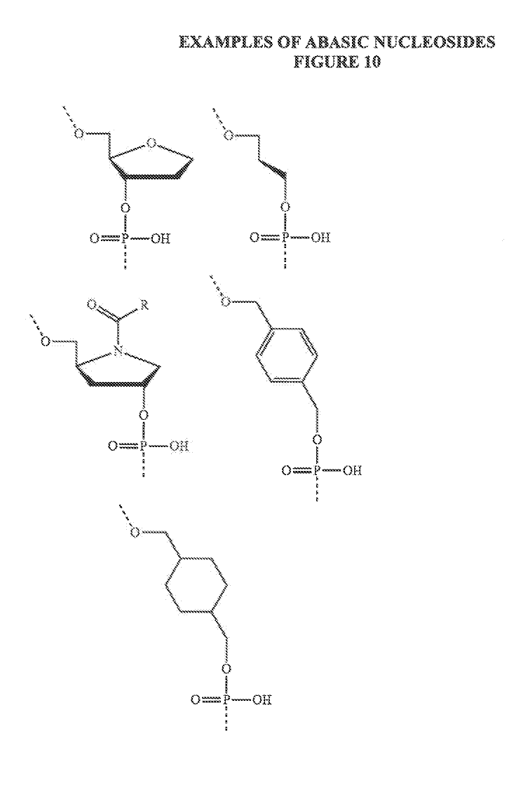

“Abasic nucleoside” refers to any of a number of structures that typically have a normal or modified nucleoside sugar and can be linked to other nucleosides. The position normally occupied by the base has some chemical moiety, such as a methylene group, that is not an isostere of any naturally occurring base found in nucleic acids and that has essentially no meaningful charge-charge or steric hindrance type interactions with any opposing nucleoside in a nucleic acid to which the strand containing the abasic nucleoside binds by means of complementary base pairing. The moiety in the sugar position in abasic nucleosides, however, can radically depart from normal ribose structure to include novel five membered or six membered rings or no ring at all as shown in FIG. 10.

“Accommodating Guide Strand Design” or “AGSD” refers to the methods and compositions provided by the present invention that are not described in the prior art for ssRNAi compounds. The ssRNAi compounds generated by AGSD are generally referred to as agsRNAi.

“Accommodating guide strand modification” or “accommodating modification” or “AGSD modification” refers to one of the specific AGSD modifications provided for herein. AGSD modifications are not provided by the prior art relating to ssRNAi.

“Activity,” unless otherwise qualified, refers to the maximum ability of an agsRNAi, ssRNAi, siRNA or miRNA compound to modulate the expression and/or function of its target. It is the amount of the maximum effect on the target as determined by the plateau generated by a dose response curve for the compound in question. It is typically expressed as a percent change relative to the base line or to a negative control. Activity is to be distinguished from potency.

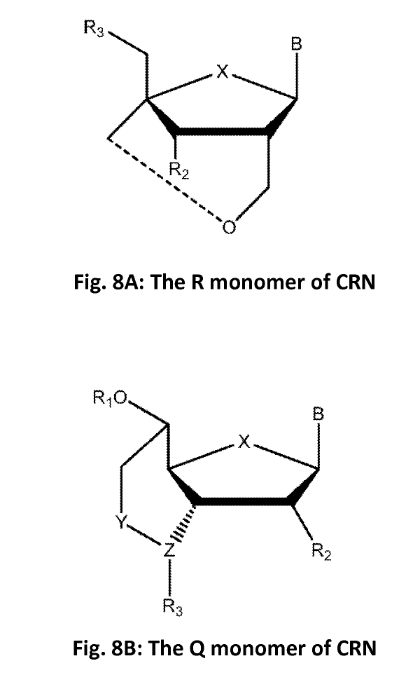

“AENA” is an abbreviation for a 2′-deoxy-2′-N,4′-C-ethylene-LNA. It is shown in FIG. 7 where B is any of the bases provided for herein.

“AGO-2 based catalytic off-target silencing activity” or any similar statement refers to situations where an ags-MiR functions as an ags-siRNA. Various methods are provided herein to inhibit such siRNA-like off-target activity.

“AgsRNAi” is a general term referring to ssRNAi compounds generated using AGSD methods and modifications.

“Ags-siRNA” refers to an agsRNAi directed to a nucleic acid target other than an miRNA. Ags-siRNAs have the same targeting code as the one found in siRNA.

“Ags-IMiR” refers to an agsRNAi directed to an miRNA target or to a non-canonical miRNAs target such as a simtron or a mirtron. Ags-IMiRs have the same targeting code as the one found in siRNA.

“Ags-MiR” refers to an agsRNAi that is an miRNA mimic. Ags-MiRs have one of the targeting codes found in miRNA. It can either mimic a particular naturally occurring miRNA or it can have a novel targeting code. Ags-MiR compounds based on a seed sequence targeting code can be based on the modular design approach.

“Algorithms” refers to sets of rules used to design specific aspects of agsRNAi compounds.

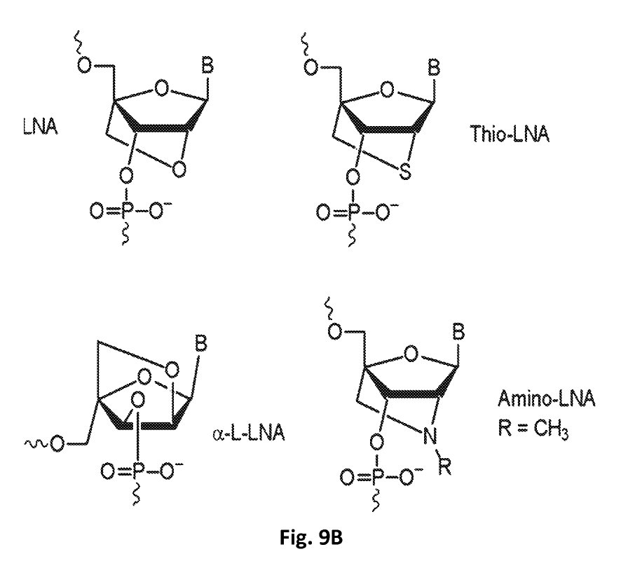

“ALN” is an acronym for α-L-LNA. It has an alpha-L-ribo configuration and is illustrated in FIG. 9 panels A and B along with one phosphodiester linkage and where B is any of the bases provided for herein.

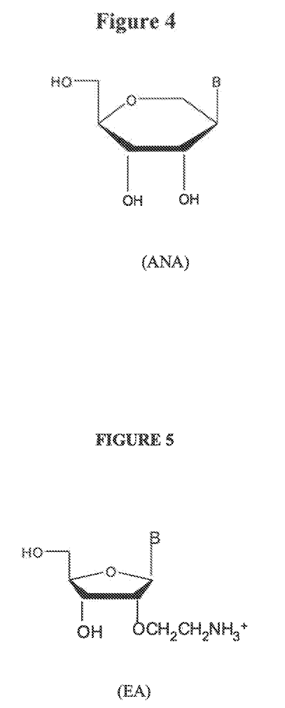

“ANA” is an acronym for altritol nucleic acid. It is illustrated in FIG. 4 where B is any of the bases provided for herein.

“Antisense oligo” or “conventional antisense oligo” when not used in the context of an RNAi drug or RNAi trigger are single stranded oligos that inhibit the expression and/or function of a targeted nucleic acid. Such antisense oligos produce their biologic effects by one of the following mechanisms: (1) Steric hindrance—e.g., the antisense oligo interferes with some step in the sequence of events involved in gene expression and/or production of the encoded protein by directly interfering with one of these steps. Such steps can include transcription of the gene, splicing of the pre-mRNA and translation of the mRNA; (2) Induction of enzymatic digestion of the RNA transcripts of the targeted gene by RNase H or other enzyme such as RNase L, RNase P or double stranded RNase; and (3) Combined steric hindrance and induction of enzymatic digestion activity in the same antisense oligo. Conventional antisense oligos do not have an RNAi mechanism of action as determined by any of a number of techniques well established in the art.

“Antisense oligo or antisense strand” when used in the context of an RNAi drug or RNAi trigger refers to the guide strand of an RNAi drug or RNAi trigger. “Argonaute” is a family of proteins or a member of the family that in humans has four members abbreviated AGO-1 through AGO-4. RISC typically has one such protein that non-covalently binds the guide strand of an RNAi trigger.

“Backbone” refers to the alternating linker/sugar structure of oligos that holds the nucleosides in a particular order.

“Base” refers to the component of a nucleoside or nucleoside analog that when incorporated into an oligo can directly engage in complementary base paring with a nucleic acid. The standard naturally occurring bases (canonical bases) are cytosine, uracil, adenine, guanine and thymine. Certain non-canonical bases are AGSD modifications.

“Basic nuclease resistance” refers to the modifications described herein, other than AGSD modifications, which must be applied to agsRNAi compounds in order to achieve adequate nuclease resistance for the intended application(s). The basic nuclease resistance modifications commonly include 2′-fluoro and 2′-O-methyl sugar modifications and can include phosphorothioate and other linkages that increase nuclease resistance compared to the phosphodiester linkage. For agsRNAi compounds that will be used with a protective carrier, the level of basic nuclease resistance can be lower than for compounds that will be used without a protective carrier. The basic nucleases resistance modifications can be increased or decreased on an as needed basis for applications that involve differences in the levels of nuclease activity found in particular organs/tissues/cells and/or in particular species. Linkage sites that are cleaved by nucleases in particular organs/tissues/cells can be readily determined using a combination of liquid chromatography and mass spectrometry (LC-MS). Once a cleaved linkage site(s) is identified the nuclease protection can be increased by changing one or both of the sugars in the linkage site to a more resistance sugar, including an AGSD sugar, and/or by replacing a phosphodiester linkage with a phosphorothioate linkage or another linkage providing nuclease resistance or by replacing a phosphorothioate linkage with one of the other nuclease resistant linkages.

“Carriers” refer to structures that can be used to directly or indirectly facilitate the uptake and/or increase the probability that an associated agsRNAi, ssRNAi, siRNA or miRNA compound will enter those cells that are accessible to the carrier/RNAi trigger combination. As a result of the action of the carrier, the RNAi trigger becomes bioavailable in cells as evidenced by its ability to produce the intended effect(s) on its intracellular target(s). Carriers that function as transfection agents are composed of cationic lipids. They will only work in vitro and then only with susceptible cell lines or primary cell cultures. Carriers for use in subjects typically will only be able to access some organ/tissue/cell types in subjects to the exclusion of others and come in several types including the following: (1) those “protective carriers” that envelop the compound and thus provide added protection from extracellular enzymatic attack until the compound is released in cells; (2) those “non-protective carriers” that are associated with the compound but that do not envelop it. This association can be covalent or non-covalent. This type of carrier includes those that either: (a) promote the binding of the compound to plasma proteins to a degree that promotes clearance of the compound from the general circulation by cells in the body rather than clearance by the kidney; and/or (b) those that bind to a cell surface receptor that then internalizes the carrier/RNAi trigger where the RNAi trigger eventually is released into the interior of the cell. Carriers are to be distinguished from “vehicles” which are ingredients of a drug formulation other than the drug. Vehicles do not function as a carrier as defined here. They can be excipients or materials involved in controlling the rate of release of the RNAi trigger or other drug into extracellular fluids.

“Cell line” refers to a population of cells taken from a tissue or an abnormal growth in a subject that has been passaged at least once in cell culture. With each subsequent passage (subculture), the cell population becomes more homogeneous as the faster growing cells come to predominate.

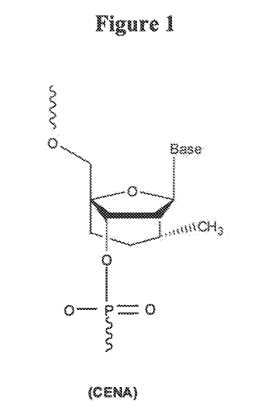

“CENA” is an acronym for 2′,4′-carbocyclic-ethylene-bridged or 2′,4′-carbocyclic-ENA-locked nucleic acid. It is illustrated in FIG. 1 where “base” represents any of the bases provided for herein.

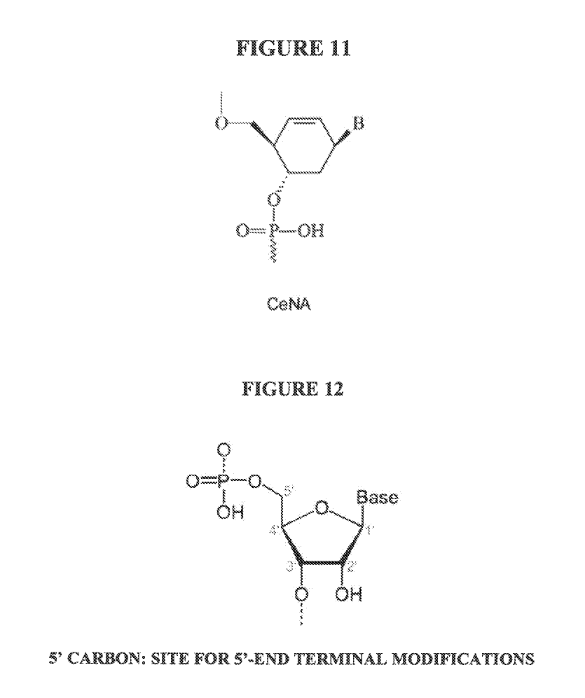

“CeNA” is an acronym for cyclohexenyl nucleic acid. It is illustrated in FIG. 11 where “base” represents any of the bases provided for herein.

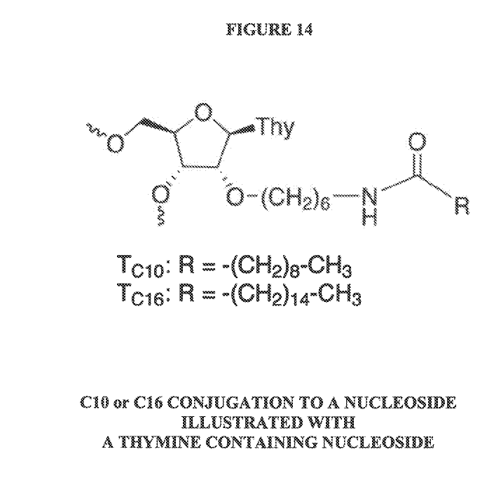

“cEt” is Ethyl bicyclic nucleic acid. See FIG. 13B for the structure.

“Chimeric oligonucleotides” or “chimeric oligos” are ones that contain ribonucleosides as well as 2′-deoxyribonucleosides. In the case of siRNA, ags-siRNA and ags-siRNA the guide strand can have one or more 2′-deoxyribonucleosides in the seed sequence as one option to reduce any unintended miRNA-like activity. In the case of agsRNAi contiguous 2′-deoxyribonucleosides can be used when joined by a boranophosphate linkage.

“Cognate” refers to a double stranded siRNA or miRNA that has the same guide strand sequence and, unless otherwise stated, the same pattern of modifications as a corresponding ssRNAi and/or agsRNAi and conversely. Unless otherwise stated the passenger strand in a cognate siRNA or miRNA is fully complementary to the guide strand through nucleoside position 19 counting from the 5′-end and it is unmodified. An ssRNAi or agsRNAi typically possesses a phosphate or phosphate analog conjugated to the 5′carbon of the 5′end nucleoside while the cognate siRNA or miRNA guide strand is not required to have such a 5′-carbon modification.

“Compounds” refers to compositions of matter that include agsRNAi, ssRNAi, siRNA and miRNA, as well as to individual guide and passenger strands.

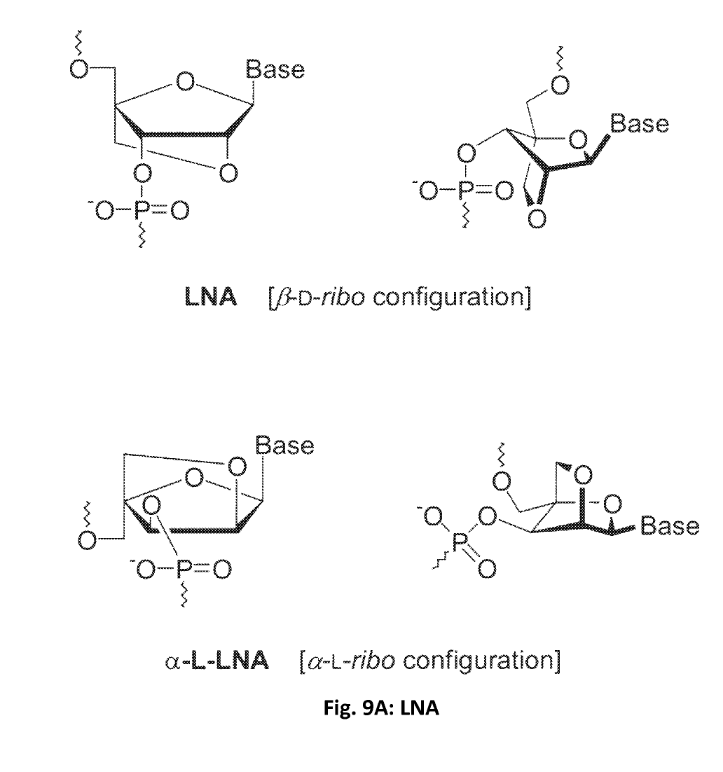

“CRN” is an acronym for conformationally restricted nucleoside or nucleomonomer as described in WO 2011/139710 except the linkages used to connect CRNs to other nucleosides in accordance with the present invention are not necessarily phosphodiester. CRN nucleosides come in two basic forms illustrated in FIG. 8. In the R monomer, X can be independently selected for each occurrence in an agsRNAi compound from the group consisting of O, S, CH₂, C═O, C═S, C═CH₂, CHF or CF₂; R₂ and R₃ are the linkages and B is a nucleobase or nucleobase analog where the linkages, nucleobases or nucleobase analogs are independently selected from the group consisting of those provided for herein. In the Q monomer X and Y can be independently selected for each occurrence from O, S, CH₂, C═O, C═S, C═CH₂, CHF or CF₂; R₁ and R₃ are the linkages, R₂ is independently selected for each occurrence from the group consisting of H, F, OH, OCH₃, OCH₃OCH₃, OCH₂CH₃OCH₃, CH₂CH₃OCH₃, CH(OCH₃)CH₃, allyl although H, F, OH, OCH₃ are preferred; Z is independently selected for each occurrence from the group consisting of N or CH; and B is a nucleobase or nucleobase analog where the linkages, nucleobases or nucleobase analogs are independently selected from the group consisting of those provided for herein.

“Dicer” is a protein that has various capabilities including an enzymatic activity that cleaves double strand RNA precursors to generate siRNA or miRNA compounds with 3′-end overhangs in cells. It also is typically a component of the RLC. It is capable of binding both double and single strand RNA.

“Dose” is given as milligrams or another unit of weight based on grams of a compound typically divided by the weight of the recipient subject assumed to be a kilogram such as milligrams per kilogram (mg/kg).

“Drug” can refer either to a pharmaceutical grade product meeting FDA standards for being called a drug or to a product not manufactured or synthesized to such standards but that could be. The latter compounds can be used for non-clinical research purposes.

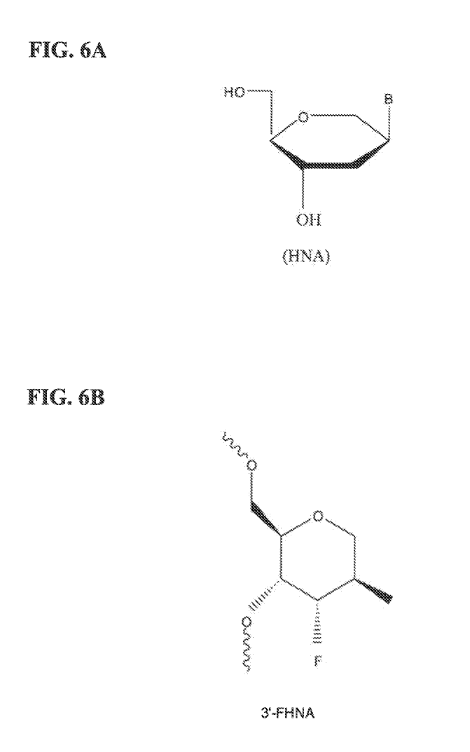

“EA” is an abbreviation for 2′-aminoethyl nucleoside. It is illustrated in FIG. 5 where B represents any of the bases provided for herein.

“Enveloping carrier,” “protective carrier” or similar phrase refers to a carrier that surrounds one or more RNAi triggers protecting them from enzymatic attack in extracellular fluid(s) and that promotes their uptake by one or more organ/tissue/cell types in the body of a subject.

“FANA” or 2′-deoxy-2′fluoro-arabinonucleic acid refers to a nucleoside modification where the fluorine has the same stereochemical orientation as the 2′-hydroxyl in β-D-arabinofuranose. In instances where the fluorine has the same orientation as the 2′-hydroxyl in β-D-ribofuranose, the associated nucleoside is referred to as 2′-fluoro.

“F-CeNA” is an acronym for fluoro cyclohexenyl nucleic acid. The basic CeNA structure is illustrated in FIG. 11 where “base” represents any of the bases provided for herein. In the case of F-CeNA, which is not illustrated, the fluorine appears in the 2′ position while the base is in the 1′ position.

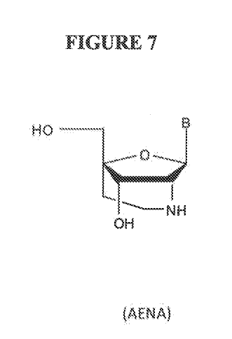

“FHNA” or “3′-FHNA” is an abbreviation for 3′-fluoro hexitol nucleic acid. The basic HNA nucleoside structure is shown in FIG. 6 where B is one of the bases provided for herein.

“Flexible sugar” is used as is or with modifiers that indicate degrees of flexibility in terms of the ease with which a sugar in a nucleoside changes its conformation (pucker) in response to outside influences.

“General circulation” or “systemic circulation” refers to the blood circulated throughout the body by means of the heart and the associated system of blood vessels. Compounds such as agsRNAi, ssRNAi, siRNA and miRNA that are administered to a subject in such a way that they enter the general circulation for distribution to their target cells are said to be systemically administered. Suitable means of administration include, but are not limited to, intravenous, intra-arterial, intradermal, intramuscular and by inhalation. Administration to those areas of the body that are protected by barriers that limit the passages of such compounds out of the general circulation and into regions of the body such as the central nervous system, testicles, placenta, fetus and the interior of the eye (aqueous and vitreous humor) typically require by passing the barrier for example by injecting a compound intraventricularly, intrathecally or intranasally.

“Guide strand” is used interchangeably with antisense strand in the context of agsRNAi, miRNA, siRNA or ssRNAi compounds. In contrast, antisense oligos or conventional antisense oligos refers to a different oligo-based drug class.

“Gymnosis” a method of gaining cellular uptake of oligos by cells in culture to effect changes in the expression and/or function of an intracellular target(s) where the uptake is achieved by prolonged incubation of the cells with high concentrations of the oligos. Gymnosis allows oligo uptake to occur that is unassisted by transfection agents or any mechanical means such as electroporation. It is applicable to numerous cell types that are not susceptible to these methods allowing them to be successfully treated with intracellular target modulating oligos (Stein et al., Nucleic Acids Res 38, No. 1 e3: 1-8, 2010-published online Oct. 23, 2009).

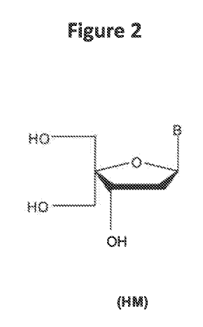

“HM” is an abbreviation for the 4′-C-hydroxymethyl-DNA nucleoside shown in FIG. 2 where B is one of the bases provided for herein.

“HNA” is an abbreviation for hexitol nucleic acid and includes the nucleoside shown in FIG. 6A where B is one of the bases provided for herein and it is conjugated to the 2′ position carbon of the ring.

“Inhibit expression” or “inhibition of expression” refers to a reduction in the level of expression of a target nucleic acid or its product. It can involve, for example, a reduction in target RNA levels, demonstrated degradation of an RNA target, a reduction in an gene transcript, reduction in protein synthesis where the target is an mRNA encoding the protein or all of the above.

“Internal linkage sites” refers to linkage sites that are not at the very 5′ or 3′-end of an agsRNAi or ssRNAi. These sites are potentially subject to single strand endonuclease attack. Along with the very 5′- and 3′-end linkages, internal linkage sites may also be simply referred to as linkage sites.

iPS cell or iPSC are abbreviations for induced pluripotent stem cells. They are created (induced) from somatic cells by experimental manipulation. “Pluripotent” refers to the fact that such stem cells can produce daughter cells committed to one of multiple possible differentiation programs.

“Kit” refers to the drug combination of an agsRNAi and its passenger strand where these two drugs are administered sequentially typically to a subject. It is not meant to imply that these two drugs have to be packaged and/or sold together as a single unit. Kits can also comprise agsRNAi of the invention in single stranded from in the absence of a passenger strand.

“Linkage site” refers to a particular linkage site or type of linkage site within an agsRNAi, ssRNAi or other oligo. A linkage site is defined by its the linkage and the identities of the contiguous 5′ and 3′ nucleosides or in the case of an overhang precursor the units. Linkage sites generally can be designated by “X-Y” where X and Y each represent nucleosides with one of the normal bases (A, C, G, T or U) or other bases provided for herein or nucleosides and the dash indicates the linkage between them.

“LNA” is the acronym for locked nucleic acid. Standard LNA and three common variants are illustrated in FIG. 9 along with one phosphodiester linkage where “base” or B represents any of the bases provided for herein. These are standard LNA, thio-LNA and amino-LNA and alpha-L-LNA. The latter also can be referred to as ALN. Unless otherwise stated when LNA is referred to it should be interpreted as referring to standard LNA.

“Mismatch” refers to a nucleoside in an agsRNAi or ssRNAi compound that does not undergo complementary base pairing with the target(s) of the compound.

“MicroRNAs (miRNAs)” are a category of naturally occurring RNAi trigger that typically cause the post-transcriptional repression of protein encoding genes after the guide strand is loaded into RISC. Uncommonly, some miRNAs can also cause the increased expression of their RNA target and/or modulate the function of their target(s) in a positive or negative direction. The miRNA guide strand directs RISC to specific RNA targets as recognized by the targeting code. Most commonly the seed sequence recognizes completely matched sequences in the 3′UTR of mRNAs transcribed from multiple different genes.

“MicroRNA mimics or miRNA mimics” are a category of manufactured compounds that when administered to cells in vitro or cells in subjects utilize the cellular mechanisms involved in implementing the activity of naturally occurring miRNA in order to produce a modulation in the expression and/or function of a particular set of RNA and/or gene targets. MicroRNA mimics of the present invention (ags-MiRs), like conventional double strand miRNA mimics, can be designed to modulate some or all of the same targets modulated by a particular naturally occurring miRNA. When based on an endogenous miRNA, an ags-MiR will have the same targeting code as the endogenous miRNA. The rest of the compound may have all, some or none of the sequence found in the endogenous miRNA from which the targeting code sequence was taken. Ags-MiRs also can be designed to modulate the expression of a set of RNA and/or gene targets by using a novel targeting code sequence not found in any know endogenous miRNA.

“miRNA” refers to one of the two major types of double strand RNAi triggers. The other major type is siRNA. miRNA and siRNA are structurally basically the same and they engage the RNAi mechanism and are processed in the same way but they have different targeting codes. They typically instigate different post-target engagement RNAi mechanisms that effect target expression and/or function. Mirtrons and simtrons are non-canonical miRNAs.

“Mirtron” refers to a non-canonical miRNA that has a pre-miRNA that is defined by the entire length of the intron in which it is located. It requires pre-mRNA splicing rather than the miRNA microprocessor as an initial step in its production (Havens et al., Nucleic Acids Res 40: 4626-40, 2012; Curtis et al., Wiley Interdiscip Rev RNA 3: 617-32, 2012).

“Mismatch” refers to a nucleoside in an oligo that does not undergo complementary base pairing with the nucleoside opposite to it when the oligo binds, on the basis of complementary base pairing, to another nucleic acid or to itself when it forms a hairpin. Mismatches include those that can occur between canonical bases (such as A:G, A:C, G:G, G:A, A:A, U:U, C:C and C:U), two non-canonical bases, a canonical and non-canonical base as well as between an abasic nucleoside and a canonical or non-canonical base. In addition, the mismatch can involve a nucleoside with a chemical moiety in the position of the base that is not normally part of any natural nucleic acid and where the moiety does not undergo an attractive charge/charge interaction with an opposing canonical or non-canonical base. Examples of such moieties are provided in WO 2010/011895 and include a nucleoside with a 2,4 difluorotoyl base and a nucleoside with a 5-nitroindole base.

“Modification” is a structure added to or inserted during the manufacture or synthesis of an RNAi trigger or RNAi drug. Unmodified double strand and single strand RNAi triggers are oligoribonucleotides where the oligoribonucleotide is comprised of some combination of the common naturally occurring ribonucleosides (adenosine, guanosine, uridine, and cytidine) joined by phosphodiester linkages. In addition, natural double strand triggers and most chemically generated ssRNAi compounds have a 5′-end phosphate. 5′-end phosphate substantially boosts the activity of ssRNAi but this moiety is not necessary for siRNA or miRNA.

“Modified linkage” refers to a linkage between nucleosides and/or units provided for herein that is not a phosphodiester.

“Modulate,” “modulating” or “modulation” refer to changing the rate at which a particular process occurs, inhibiting or accelerating a particular process, redirecting a particular process, and/or preventing or promoting the initiation of a particular cellular process, e.g., cellular signaling, protein transport, drug efflux, cell growth, morphological alterations, differentiation etc.

“Modular design” or “modular approach” refers to an approach for generating ags-MiRs or ssRNAi compounds that are miRNA mimics where the targeting code is the seed sequence. According to modular design the compound is divided into three modules that can be independently manipulated where the positions of the modules are as follows: (1) the “5′-end module” consisting of nucleoside positions 1-8 or 1-9; (2) the “seed vehicle” consisting of nucleosides 9-19 or 10-19 depending on the length of the 5′-end module; and (3) an optional overhang precursor comprising nucleosides and/or units starting in position 20 and continuing up to position 23 depending on its length.

“Moiety” refers to a part of a molecule, such as a nucleoside, sugar, base, linkage, agsRNAi or ssRNAi, which exhibits a particular set of chemical and/or pharmacologic properties.

“Newer ssRNAi” refers to the ssRNAi compositions described in Haringsma et al., Nucleic Acids Res 40: 4125-37, 2012; Yu et al., Cell 150: 895-908, 2012; Chorn et al., RNA 18: 96-804, 2012; Lima et al., Cell 150: 883-94, 2012; WO 2011/046983; WO 2011/139699; WO 2011/139702; and WO 2012/027206.

“Nuclease resistant linkage” refers to one of the linkages provided herein that are more nuclease resistant than phosphodiester.

“Nucleoside” refers to any one of the structures that can appear in positions 1-19 counting from the 5′-end of the RNAi triggers described herein. These structures include natural nucleosides, nucleosides with non-canonical basis and/or sugar analogs or sugar substitutes as well as abasic nucleosides that can lack any ringed structure as shown in one example in FIG. 10. Leaving aside the linkages overhang precursors consist in part or entirely of nucleosides. The structures in overhang precursors that are nucleosides can also be called units while other component structures that are not nucleosides are necessarily called units.

“Off-target silencing or effects due to the seed sequence” or any similar statement refers to situations where the seed sequence an RNAi trigger intended to only have siRNA or siRNA-like activity directs RISC to unintended targets that are then silenced by a miRNA mechanism. Various methods are provided herein to inhibit such seed sequence based off-target activity by agsRNAi. These include using a nucleoside in position 2 that has a 2′ modification of the size of a methyl group or larger and/or by using modifications provided herein that reduce the affinity between the seed region and the unintended targets. Theses modification(s) can reduce the off-target inhibition of 1, 2, 3, 4, 5 or greater than 5 targets engaged by the seed region by 20, 30, 40, 50, 60, 70, 80, 90 or >90% compared to compounds without the modification.

“Oligonucleotide” or “oligo” refers to a strand of ribonucleosides and/or ribonucleoside analogs and/or 2′-deoxyribonucleosides and/or 2′-deoxyribonucleoside analogs comprising about 13 to 30 nucleosides that are linked by phosphodiester and/or by nuclease resistant linkages.

“Oligoribonucleotide” refers to an oligo where the nucleosides are ribonucleosides or ribonucleoside analogs such as 2′-fluoro and 2′-O-methyl. A ribonucleoside is one in which the sugar is more likely in the C3′-endo conformation when tested alone or as part of a linked multi-ribonucleotide strand.

The phrase “operably linked” means that the sequences necessary are placed in the nucleic acid molecule in the appropriate positions so as to effect expression of the sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a modular nature wherein a hybrid nucleic acid molecule of modules is generated.

“Organ” is a group of tissues that are a discrete structural unit in the body that performs a specific function or set of functions. Examples include but are not limited to liver, kidney, lung, spleen, pancreas, brain, stomach, prostate, uterus, ovary, gall bladder, heart, bladder, esophagus, duodenum, jejunum, ileum, colon, testis, skin, and eye. Details and other examples are provided by standard medical texts of anatomy and histology such as “Junqueira's Basic Histology: Text and Atlas,” Anthony Mescher (Author), McGraw-Hill Medical; Thirteenth Edition, February 2013.

“Overhang,” in the context of siRNA and miRNA, refers to any portion of the passenger and/or guide strand that extends beyond the duplex formed by these strands. In the case of natural RNAi triggers, both strands have 3′-end overhangs that are typically two nucleosides in length. siRNA and miRNA drugs, however, can have 5′ and/or 3′-end overhangs on one or both strands that are 1-4 nucleosides and/or units in length or have no overhang(s).

“Overhang precursor” refers to that portion, if any, of an agsRNAi or ssRNAi that would form an overhang if combined with a 19-mer partner passenger strand capable of forming a duplex with the agsRNAi or ssRNAi nucleosides in positions 1-19. Overhang precursors are 1-4 nucleosides and/or units in length. They can engage the PAZ domain of Dicer and/or an argonaute protein but do not directly engage the agsRNAi target(s).

“Parenchyma” refers to the defining essential and distinctive cell type found in an organ, gland or neoplastic growth. In each of these instances the parenchymal cells are distinguished from its supportive framework including such elements as connective and vascular tissue and in some instances stromal cells. The parenchymal cells of a particular organ may or may not also be formally classifiable as one of the three general tissue types, i.e., epithelial, nervous or muscle. Strictly speaking some other parenchymal cells, such as hepatocytes do not fall into one of the categories of tissue types. Nevertheless, they are not uncommonly referred to by the name of the organ followed by the word tissue, e.g., liver tissue. Details and more examples are provided by standard medical texts of anatomy and histology such as “Junqueira's Basic Histology: Text and Atlas,” Anthony Mescher (Author), McGraw-Hill Medical; Thirteenth Edition, February 2013.

“Passenger strand” is used interchangeably with “sense strand” in the context of miRNA or siRNA compounds. It forms a duplex with its partner guide or antisense strand on the basis of complementary base pairing to form one of these double strand compounds.

“Pharmacologically effective amount,” “therapeutically effective amount” or simply “effective amount” refers to that amount of an agent effective to produce a commercially viable pharmacological, therapeutic, preventive or other commercial result.

“Potency” describes the activity of an agsRNAi, ssRNAi, siRNA or miRNA compound expressed in terms of the amount required to produce a given level of activity under certain defined circumstances. Potency provides a useful basis for comparing drugs with a common mechanism-of-action. Typically it is defined in terms of the molar concentration (molarity) of the compound or the amount of the compound divided by a measure of weight correlated with the subject or solid tissue sample being treated by the compound. For example, a 20-gram mouse systemically treated with 0.1 mg of an agsRNAi can be said to have been treated with 5 mg/kg of the compound. A higher potency compound evokes a larger response at a given molar concentration or amount per subject (or tissue sample weight) while a lower potency compound evokes a smaller or no response at the same total amount as the higher potency compound when administered under the same experimental conditions. Potency is typically measured using a dose response curve for the compound administered in vitro or to a subject and measured a specified time after the last treatment with the compound. The half maximal inhibitory concentration (IC₅₀) is a measure of the effectiveness of a compound in inhibiting the expression and/or function of its target. The half maximal effective concentration (EC₅₀) is a measure of the effectiveness of a compound in modulating the expression and/or function of its target. EC₅₀, however, is typically used to describe the effect of a compound that promotes the expression and/or function of the target. IC and EC measurements can also be used for other levels of activity where an alternative percent of the maximal level of change replaces the subscripted 50, for example IC₇₅ would represent 75% of the maximal inhibitory activity. Two different compounds directed with the same mechanism-of-action can have the same level of activity, for example, both can suppress the expression of their target by 90% in a subject but they can very greatly in potency, i.e., one produces the 90% suppression at 1 mg/kg while the other does so at 100 mg/kg. In this example there would be a 100× difference in potency.

“Primary cells” are cells taken directly from a tissue or neoplastic growth in a subject and placed in tissue culture and that have not been passaged. A primary cell culture may be composed of mixtures of cell types, however, appropriate laboratory procedures often can be used to enrich a desired cell type, such as the parenchymal cells, from the mixture.

“Prodrug” refers to a compound that is administered to a subject in a form that is inactive but becomes active in the body after undergoing chemical modifications typically through intracellular metabolic processes.

“Protective carrier” or “enveloping carrier” refers to a type of carrier, such as a lipid nanoparticle, that can provide protection to RNAi triggers by enveloping them. This is particularly important for those triggers that lack sufficient intrinsic resistance to extracellular enzymes to allow them to be administered systemically. Such protection lasts until the RNAi trigger is released inside of cells.

“Purine rich region,” unless otherwise stated herein, is defined as a sub-region within nucleoside positions 2-19 of an agsRNAi where there are 3 or more contiguous nucleosides that have a purine base or 4 contiguous nucleosides where 3 of them have a purine base.

“Ribose” refers to β-D-ribofuranose.

“RISC” stands for “RNA induced silencing complex.” It typically includes multiple molecular components including an argonaute protein. The guide strand of an RNAi trigger is loaded into RISC often with the assistance of a RLC and this strand guides RISC to the target(s) to be engaged by the RNAi mechanism.

“Risk loading complex” (RLC) refers to a molecular complex that can bind a double or single strand RNAi trigger and subsequently present it to RISC in a manner that promotes the loading of RISC with the guide strand.

“RNAi” is an abbreviation for “RNA mediated interference” or “RNA interference.” It refers to the system of cellular mechanisms (RNAi mechanism) that can produce RNAi triggers and that engages the guide strand of an RNAi triggers for modulation of the expression and/or function of susceptible nucleic acid targets.

“RNAi activity” refers to the action of the RNAi mechanism on its target(s) as directed by the guide strand of the RNAi trigger.

“RNAi drug” strictly speaking refers to a pharmaceutical grade therapeutic but loosely can be used to describe a non-pharmaceutical grade manufactured or synthesized RNAi trigger used in non-clinical research.

“RNAi mechanism” or “RNAi machinery” refers to the cellular molecules and processes primarily involved in generating RNAi triggers and/or discarding the passenger strand while the guide strand is loaded into the argonaute component of RISC and/or the engagement by the guide directed RISC and the mechanisms and molecule involved in producing a change in the expression and/or function of the nucleic acid target(s).

“RNAi trigger” refers to single (agsRNAi and ssRNAi) and double stranded (siRNA and miRNA) oligoribonucleotides or chimeric oligos most commonly in the 19-23-mer-size range that directs the RNAi mechanism to particular targets. In the case of a double stranded RNAi trigger the passenger strand (sense strand) is discarded at some point during the process of loading the guide strand into the argonaute component of RISC. The guide (antisense strand) then directs the RNAi mechanism to its target(s). This term applies to naturally occurring siRNA and miRNA as well as to manufactured or synthesized siRNA, miRNA, agsRNAi and ssRNAi. The manufactured or synthesized compounds can exist as either pharmaceutical grade drugs or as non-pharmaceutical grade drugs for uses such as non-clinical research.

“Seed sequence” or “seed region” is the region comprised of the 6-7 contiguous nucleosides in positions 2-7 or 2-8 counting in from the 5′-end of the guide strand of an RNAi trigger. In the case of a miRNA, ssRNAi that functions as a miRNA mimic or an ags-MiR it is also referred to as the “targeting code.”

“Seed vehicle” is used to describe the region of an ags-MiR or ssRNAi constructed according to the modular design approach other than those nucleosides in positions 1-9 or 1-8 comprising the 5′-end module and other than any overhang precursor. Hence it is comprised of the nucleosides in positions 10-19 or 9-19 depending on the length of the 5′-end module.

“Significant” or “significantly” when used in the context of a change in the level of function in a RNAi trigger as a result of some modification or set of modifications refers to an incremental change in activity, potency and/or duration of effect on a target having a minimum incremental change as provided for herein for comparing one agsRNAi compound to another or to an ssRNAi with the same sequence but with modifications known in the prior art. These minimal incremental changes are stated in the Summary of the Invention. In other instances “significant” refers to statistical significance.

“Silencing” or “silencing activity” traditionally refers to the inhibition of target expression and/or function that occurs as a result of RNAi activity. It is now clear, however, that RNAi activity can sometimes boost the expression and/or function of its target(s). So any such enhancing activity is included under silencing as used herein.

“Simtron” refers to a non-canonical microRNA similar to mirtrons that do not require splicing for their biogenesis (Havens et al., Nucleic Acids Res 40: 4626-40, 2012).

“seqRNAi” refers to technology first described in WO 2011/046983 and WO 2012/145729 where the passenger and guide strands of an RNAi trigger can be administered sequentially to a subject at a sufficient interval to allow the first strand to be taken up by a wide range of organ/tissue/cell types before the second is administered. This provides a means for achieving RNAi activity in most organ/tissue/cell types in the body without the need for a carrier. As for agsRNAi, seqRNAi compounds are divided into three basic types: (1) those that have an siRNA targeting code but target some nucleic acid other than miRNA (seq-siRNA); (2) those that have an siRNA targeting code and target miRNA (seq-IMiRs); and (3) those that have an miRNA targeting code and mimic an endogenous miRNA or have a novel miRNA targeting code sequence.

“siRNA” refers to one of the two major types of double strand RNAi triggers. The other major type is miRNA. miRNA and siRNA are structurally basically the same and they engage the RNAi mechanism and are processed in the same way but they have different targeting codes. They typically instigate different post-target engagement RNAi mechanisms that effect target expression and/or function.

“ssRNAi” or “ss-RNAi” refers to a guide strand only RNAi trigger that does not have to be used with a passenger strand.

“Stem cell” refers to a rare cell type in the body that exhibits a capacity for self-renewal. When a stem cell divides the resulting daughter cells are either committed to undergoing a particular differentiation program or they are the product of self-renewal of the parent stem cell in which case they constitute a replica of the parent stem cell. By undergoing self-renewal, stem cells function as the source material for the maintenance and/or expansion of a particular tissue or cell type.

“Subject” refers to mammals and in particular those commonly treated with therapeutic agents and/or commonly used as models during the development of therapeutic agents. Subjects include dogs, cats, rabbits, mice, rats, hamsters, guinea pigs, miniature pigs, ferrets, non-human primates and humans as well as farm animals including horses, cattle, pigs, sheep and chickens. Subjects include individuals who are normal, suffering from a particular medical disorder, genetically modified or are models of a particular medical indication.

“Subject cells” or a similar phrase refers to one or more particular cell types found in subject or derived from a subject. Such cells can be selected from the group consisting of the following: a particular cell line; a representative sampling of a particular organ, gland or type of neoplastic growth; an enriched sample of parenchymal cells from a particular organ, gland or type of neoplastic growth; a particular type of epithelial tissue including simple, stratified, pseudostratified and transitional; a particular type of connective tissue where the types are loose and dense forms with the latter being further subdivided into dense regular and dense irregular, other types of connective tissue include reticular connective tissue, adipose tissue, blood and lymphoid tissue; a particular type of nervous tissue subdivided by location (such as brain, spinal cord, ganglion, and nerve) and/or by cell type, i.e., neuronal or glial; and a particular type of muscle tissue where the types are skeletal, cardiac and smooth. Cells of one of these general types can be further subdivided into subsets of cells with defined molecular and/or functional characteristics by methods known in the art. Further, subject cells can be normal or abnormal. Unless otherwise stated or implied by the context the term “subject cells” applies to both normal and abnormal cells. Distinguishing between subject cell types is important for comparing the effects of particular agsRNAi, agsRNAi/passenger strand combinations and ssRNAi compounds on their targets and the relative abilities of particular cell types to take up and metabolize such compounds.

“Sugar,” “sugar analog,” “sugar substitute” or “modified sugar” refers to the component of a nucleoside that occupies the functional position of a ribose or 2′-deoxyribose sugar in a naturally occurring nucleoside. For simplicity the word sugar as used herein can refer to a sugar analog, sugar substitute, or modified sugar as well as to actual sugars. Thus, the word sugar can be applied to a sugar analog, sugar substitute, or modified sugar that is a AGSD modification as well as to sugars that are not AGSD modifications. Examples of sugars that are not AGSD modifications are normal ribose and 2′-deoxyribose as well as 2′-fluoro, 2′-O-methyl and 2′-methoxyethyl ribose modifications. Hence the term “sugar” as it is used herein is not meant to have the same definition as the term sugar that is used in chemistry.

“Synthetic” means chemically or enzymatically manufactured or produced by man and isolated for use as a drug.

“Targeting code” refers to the portion of the guide strand sequence in an agsRNAi, ssRNAi, siRNA or miRNA that is primarily or exclusively responsible for directing RISC to a specific target(s). The term does not refer to any particular sequence of nucleosides. Targeting codes typically can be distinguished on the basis of their positions within the guide strand relative to its 5′-end. Ags-MiRs as well as ssRNAi compounds with miRNA activity and miRNA itself have one of two possible targeting codes. One of these targeting codes is referred to as the seed sequence. The seed sequence comprises either 6 or 7 consecutive nucleotides beginning at position 2 counting from the 5′-end and running to position 7 or 8. The second targeting code is 11 or 12 consecutive nucleosides in length beginning at position 4 or 5 from the 5′-end although optionally position 8 or 9 can have a base mismatch with the target. Ags-siRNA, ags-IMiRs as well as ssRNAi compounds with siRNA activity and siRNA itself have a targeting code that is 15 consecutive nucleosides in length extending from position 2 through position 16 counting from the 5′-end although optionally position 8 or 9 can have a base mismatch with the target.

“Target nucleic acid” refers to a target for an RNAi trigger. With respect to the agsRNAi such targets include but are not limited to genes, mRNA, miRNA and other regulatory non-coding RNA. Despite common usage of “non-coding RNA,” it has been discovered that some non-coding RNA actually are coding in at least some cellular environments. AgsRNAi non-coding RNA targets include but are not limited to lncRNA, promoter associated RNA, enhancer RNA, snoRNA, piRNA, xiRNA, sdRNA, moRNA, MSY-RNA, tel-sRNA, crasiRNA and endogenous antisense RNA. Some agsRNAi can produce a change in expression of and/or modulate the function of genes by directly engaging DNA targets on the basis of complementary base pairing including but not limited to one or more entities controlling particular gene(s) and selected from the group promoters, enhancers or suppressors.

“Tissue” is an aggregation of similar cells that together perform a certain specialized function(s). Tissues can broadly be classified into 4 basic types, which are epithelial, connective, nervous and muscle. There are two types of epithelial tissues: (1) Those that cover the outside surfaces of the body and line internal organs. This type protects the body from things such as moisture loss, bacteria and trauma; and (2) glandular epithelium that secretes products such as hormones, stomach acid, sweat, saliva and milk. Connective tissue provides structure and support to the body. The types of connective tissue can be broadly subdivided into connective tissue proper, special connective tissue and a series of other special types of connective tissue. Connective tissue proper consists of loose and dense forms with the latter being further subdivided into dense regular and dense irregular. The special types of connective tissue include reticular connective tissue, adipose tissue, cartilage, bone, blood and lymphoid. Nervous tissue occurs in two types, which are neurons and neuroglia. Muscle tissue occurs as skeletal, cardiac and smooth types. Organ samples are also sometimes loosely referred to by using the name of the organ with the word tissue, for example, liver tissue or lung tissue. Strictly speaking, however, the definitive cell types in an organ are referred to as parenchymal cells. Parenchymal cells of the liver, for example, are hepatocytes and strictly speaking they do not fall into any of the four formally defined types of tissue. Details and additional examples are provided by standard medical texts of anatomy and histology such as “Junqueira's Basic Histology: Text and Atlas,” Anthony Mescher (Author), McGraw-Hill Medical; Thirteenth Edition, February 2013.

“Tm” or melting temperature is the midpoint of the temperature range over which an oligo separates from a complementary nucleotide sequence or where complementary sequences within the oligo itself (hairpin) are separated. At this temperature, 50% hybridized and 50% unhybridized forms are present. Tm can be measured by using the UV spectrum to determine the formation and breakdown (melting) of hybridization using techniques that are well known in the art. There are also formulas available for estimating Tm on the basis of nearest neighbor analysis or in the case of very short duplexes in accordance with the relative G:C and U:A content. For the purposes of the present invention Tm measurements are based on physiological pH (about 7.4) and salt concentrations (about 150 mM).

“Transfection” refers to a method for facilitating the passage of oligos, such as RNAi triggers, across the membrane of susceptible cells grown in tissue culture. The method involves incubating the oligo(s) with a transfection agent and then applying the mixture to the surface of cells for a period of time followed by the removal of the transfection agent by washing. The transfection agents are cationic lipids that form liposomes that fuse with the membranes of cells and deposit the RNAi trigger inside them. Such transfection agents are not suitable for use in subjects and are to be distinguished from carriers that are used for that purpose. “Treatment” refers to the application or administration of an RNAi trigger or RNAi drug to a subject or patient, or to an isolated tissue, primary cell sample or cell line from a subject or patient.

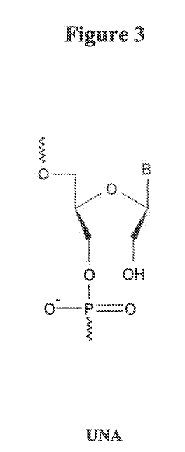

“UNA” is an acronym for unlocked nucleic acid or nucleoside. The ribose sugar ring in the nucleoside is acyclic by virtue of lacking the bond between the 2′ and 3′ carbon atoms as shown in FIG. 3 where B represents any of the bases provided for herein.

“Unacceptably stimulates innate immune response” or any similar statement refers to situations where certain nucleosides in the ssRNAi strand, such as some of those where U, A or G are coupled with ribose or to a lesser extent 2′-fluoro, activate the release of inflammatory cytokines (such as TNF-α, IL-1, IL-6, IL-12 and IL-16) from cells as a result of stimulating receptors involved in the innate immune response such as TLR 7 and 8. Various well-established assays are available to monitor the effects of oligoribonucleotides, chimeric oligonucleotides and other compounds on innate immunity. The induction of innate immunity becomes a problem when it produces unacceptable side-effects in subjects such as fever, chills and weight loss. Methods provided herein to inhibit such immune stimulation include the selective use of the 2′-O-methyl modification. The modification(s) provided for this purpose can reduce levels of cytokine(s) induced by the innate immune system by 20, 30, 40, 50, 60, 70, 80, 90 or >90% compared to otherwise identical compounds without the modification(s).

“Unit” refers to the nucleosides and/or to linked non-nucleoside moieties that appear in overhang precursors. These non-nucleoside moieties are limited to those structures provided for herein which are only allowed in overhang precursors.

“Unless otherwise specified” or “unless otherwise provided for” refers to other specified modifications described herein that provide for a different modification(s) under certain circumstances. In these and in other instances where two or more rules specifying different modifications for the same entity, the more narrowly applicable rule (applies to fewer agsRNAi strands) dominates.

“Upstream” and “Downstream” respectively refer to moving along a nucleotide strand in a 3′ to 5′ direction or a 5′ to 3′ direction respectively.

“Vehicle” refers a substance of no therapeutic effect that does not act as a “carrier” as defined here but is used to convey an active medicine or compound such as an RNAi trigger. An example of a vehicle suitable for use with the compounds of the present invention is buffered saline. The term vehicle also includes substances in which a drug can be suspended or dissolved that provide for the release of the drug over time.

### C. The Embodiments

In one embodiment, AGSD methods and modifications are applied to ssRNAi in order to produce novel ssRNAi compounds (agsRNAi) with increased activity, potency and/or duration of the effect on the target(s). AgsRNAi targets are typically RNA and include pre-mRNA, mRNA and non-coding regulatory RNAs. Non-coding regulatory RNA targets include but are not limited to lncRNA, promoter associated RNA, enhancer RNA, snoRNA, piRNA, xiRNA, sdRNA, moRNA, MSY-RNA, tel-sRNA, crasiRNA and endogenous antisense RNA. Included among the non-coding RNA targets are those that, at least in some cellular environments, can have coding activity. Some agsRNAi compounds can produce a change in expression of and/or modulate the function of genes by directly engaging DNA targets on the basis of complementary base pairing including but not limited to one or more entities controlling particular gene(s) and selected from the group promoters, enhancers or suppressors. AgsRNAi uses include those involving therapeutics, diagnostics, target validation for drug development, functional genomics, genetic engineering and pharmacogenomics.

In a related embodiment the agsRNAi shares a targeting code with siRNA (ags-siRNA). In another related embodiment the agsRNAi is a miRNA inhibitor (ags-IMiR) that shares a targeting code with siRNA. In another related embodiment the agsRNAi is a miRNA or miRNA-like mimic (ags-MiR). Ags-MIRs can have a seed sequence taken from a known endogenous miRNA or have a novel seed sequence. The nucleosides in the seed sequence can be modified to increase or decrease their binding affinity with one or more of their target(s). Outside of the seed sequence they can have a sequence that is based on the sequence of the endogenous miRNA being mimicked or can have additional sequences based on the modular design approach. As an alternative to a seed sequence based targeting code an ags-MiR can have a targeting code that encompasses the 11 or 12 consecutive nucleosides beginning at position 4 or 5 counting from the 5′-end. In another related embodiment the agsRNAi is administered to a subject without a carrier. In another related embodiment the agsRNAi is administered to a subject with a carrier. In another related embodiment the agsRNAi is administered to cells in vitro. In another related embodiment the agsRNAi has a 5′-end phosphate or 5′-end phosphate analog. In another related embodiment the agsRNAi nucleosides in positions 1 and 2 and their intervening linkage are permissive for an intracellular kinase in a cell in a subject adding a 5′-phosphate to the 5′-carbon of the 5′-end nucleoside intracellularly. In another related embodiment the agsRNAi nucleosides in positions 1 and 2 and their intervening linkage are not permissive for an intracellular and/or extracellular phosphatase in a subject removing a 5′-phosphate or 5′phosphate analog from the 5′-carbon of the 5′-end nucleoside. Any such phosphatase effect can be limited to intracellular phosphatases by using an enveloping carrier to deliver the agsRNAi to the interior of cells in a subject. In another related embodiment the agsRNAi has an overhang precursor of 1-4 units in length. In yet another embodiment an agsRNAi can be administered to a cell in a subject or in vitro with a passenger strand that is unmodified or is modified using methods and compositions known in the art. In a related embodiment an agsRNAi that is suitable for administration to a subject without an enveloping carrier can be sequentially administered to a cell in a subject or in vitro in association with a passenger strand that is modified using methods and compositions known in the art for administration to a subject without the use of an enveloping carrier (WO 2011/046983; WO 2012/145729). In another related embodiment the agsRNAi compounds can be delivered to cells in subjects using a protective carrier that delivers the agsRNAi to some organs/tissues/cell types in the body to the exclusion of others. In still another embodiment the agsRNAi compounds can be delivered to cells in subjects using a carrier that is not protective that selectively delivers the agsRNAi to targeted organs/tissues/cell types in the body to the exclusion of others.

### D. Designing AgsRNAi Compounds

### Providing Basic Nuclease Resistance

Two groups have generated data showing that either of two different patterns of modification can provide sufficient basic nuclease resistance to obtain substantial ssRNAi activity in cultured cells and/or in mice. In both instances, the ssRNAi compounds were 21-mers and multiple compounds were tested confirming the general applicability of these designs.

In one design the modifications are as follows: (1) A 5′-end phosphate (2) 2′-fluoro nucleosides in positions 1-19; (3) An overhang precursor comprising two uridines with the 2′-O-methyl modification; and (4) All of the linkages were phosphodiester (Haringsma et al., Nucleic Acids Res 40: 4125-37, 2012; Chorn et al., RNA 18: 96-804, 2012). Compounds based on this design had to be delivered to liver cells in mice using a protective lipid nanoparticle because they were not sufficiently resistant to extracellular nucleases and because the phosphodiester linkage does not sufficiently adhere to plasma proteins to avoid rapid clearance by the kidneys when the compound is systemically administered.

In the other design the modifications are as follows:

(1) A 5′-end phosphate conjugated to either a uridine with a 2′-O-methyl modification or a thymidine with a 2′-O-methyoxythyl modification. Either of these gave the best results when the ssRNAi compounds were transfected into a cell line. When administered to mice, however, the 5′-end phosphate was removed in the liver and these compounds were not active. The investigators were able to get activity in the liver of animals by using a 5′-end phosphate analog called 5′-VP conjugated to a thymidine with a 2′-O-methyoxythyl modification. It is much less active than 5′-end phosphate, however, in cultured cells;
(2) 2′-fluoro alternating with 2′-O-methyl nucleosides in positions 2-19 with the 2′-fluoro being in the even numbered positions;
(3) An overhang precursor comprising two adenosines with the 2′-O-methyoxythyl modification was present with phosphorothioate linkages connecting them together as well as to the rest of the ssRNAi; and
(4) having phosphorothioate linkages between nucleoside positions 1-2, 2-3, 4-5, 6-7, 8-9, 10-11, 12-13, 14-15, 15-16, 16-17, 17-18 and 18-19 with phosphodiester linkages in positions 3-4, 5-6, 7-8, 9-10, 11-12 and 13-14 (Lima et al., Cell 150: 883-94, 2012; Yu et al., Cell 150: 895-908, 2012). Thus, there was a total of 14 phosphorothioate linkages in these ssRNAi compounds. They were administered to mice either without a carrier or with a C16 non-enveloping carrier that increased the binding of these compounds to plasma proteins. The effect of this was to reduce the clearance of these compounds from the plasma by the kidneys and thereby allowing more of the compound to be taken up by cells in the mice.

These designs for providing basic nuclease resistance to ssRNAi compounds can be used as a starting point for providing basic nuclease resistance to agsRNAi compounds, but these designs do not have to be rigidly followed. The modifications provided for nucleoside positions 2-19 by these designs in particular are a useful, but not necessary, starting point for the generation of agsRNAi compounds.

Once a particular set of modifications providing basic nuclease resistance are provided then the AGSD modifications are added. In most instances AGSD modifications would provide enhanced nuclease protection relative to the basic nuclease protection for the linkage sites where they appear.

The level of basic nuclease resistance for agsRNAi compounds is directly related to how and where the compounds are to be administered. When they are to be systemically administered with or without a protective carrier the basic nuclease resistance provided must be sufficient to protect the compounds from extracellular nucleases including those in plasma. When they are systemically administered with a protective carrier the level of basic nuclease protection can be less.

The methods for providing basic nuclease resistance described below are limited to nucleoside positions 2-19. Options for modifying the nucleoside in position 1 are provided in the section “Designing AgsRNAi Compounds: 5′-End Modifications.” Options for overhang precursors are provided in the section “Designing AgsRNAi Compounds: Overhang Precursors.”

Providing basic nuclease resistance to nucleoside positions 2-19 of agsRNAi compounds to be used with a protective carrier can be achieved by one of the following sets of modifications:

Set 1: All of the nucleosides in positions 2-19 can be 2′-fluoro and all of the linkages phosphodiester.

Set 2: The nucleosides in positions 2-19 can be 2′-fluoro alternated with 2′-O-methyl nucleosides with the 2′-fluoro being in the even numbered positions.

Set 3: As a variant of Set 2: To reduce any undesirable seed sequence dependent off-target effects on the part of ags-siRNA or ags-IMiR compounds a 2′-O-methyl modification can be used for nucleoside position 2. It is important for potential steric hindrance reasons that the nucleoside in position 14 in particular and to a lesser extent the nucleoside in position 16 not have a 2′-O-methyl or larger 2′-ribose modification. Thus, when a 2′-O-methyl modification is used for the nucleoside in position 2 it is necessary that two contiguous nucleosides in the region defined by positions 3-13 have a 2′-fluoro modified sugar so that when the alternating pattern of single 2′-O-methyl with single 2′-fluoro modified sugar is continued the 2′-fluoro modification will fall on nucleoside positions 14 and 16.

Set 4: As a variant to Set 3 the nucleosides in position 14 can be ribose or HM and optionally the nucleoside in position 16 can also be ribose or HM.

Set 5: Phosphorothioate linkages can be added to linkage sites 2-3, and to linkage site 18-19 modified while the other modifications are according to Set 1, Set 2 or Set 3 when there is a phosphorothioate in linkage site 1-2 and there is an overhang precursor where the units are linked to each other and to nucleoside 19 by phosphorothioates.

Set 6: Additional phosphorothioate linkages can be added to those appearing in Set 5 in the following linkages sites (U-G, U-A, C-A, C-C, U-C, C-G, A-C and A-U) but preferably the added phosphorothioates are not contiguous when they are in the targeting code.

Providing basic nuclease resistance to nucleoside positions 2-19 of agsRNAi compounds to be used with or without a protective carrier can be achieved by one of the following sets of modifications:

Set 1: 2′-fluoro alternating with 2′-O-methyl nucleosides in positions 2-19 with the 2′-fluoro being in the even numbered positions where phosphorothioate linkages occur between nucleoside positions 2-3, 4-5, 6-7, 8-9, 10-11, 12-13, 14-15, 15-16, 16-17, 17-18 and 18-19 with phosphodiester linkages in positions 3-4, 5-6, 7-8, 9-10, 11-12 and 13-14.

Set 2: A variant of Set 1 where there is a 3 or 4 unit overhang precursor where the units are linked by phosphorothioate linkages to each other and to the nucleoside in position 19 then optionally the phosphorothioate linkages occur between nucleoside positions 2-3, 4-5, 6-7, 8-9, 10-11, 12-13, 14-15, 16-17, 17-18 and 18-19 with phosphodiester linkages in positions 3-4, 5-6, 7-8, 9-10, 11-12, 13-14 and 15-16.

Set 3: Optionally the pattern of 2′-fluoro alternating with 2′-O-methyl nucleosides can be adjusted when an AGSD sugar modification replaces a 2′-fluoro modified sugar. In such an instance one or both of the contiguous 2′-O-methyl sugars can be changed to 2′-fluoro.

Set 4: To reduce any undesirable seed sequence dependent off-target effects on the part of ags-siRNA or ags-IMiR compounds a 2′-O-methyl modification can be used for nucleoside position 2. It is important for potential steric hindrance reasons that the nucleoside in position 14 in particular and to a lesser extent the nucleoside in position 16 not have a 2′-O-methyl or larger 2′-ribose modification. Thus, when a 2′-O-methyl modification is used for the nucleoside in position 2 that two contiguous nucleosides in the region defined by positions 3-13 have a 2′-fluoro modified sugar so that when the alternating pattern of single 2′-O-methyl with single 2′-fluoro modified sugar is continued the 2′-fluoro modification will fall on nucleoside positions 14 and 16.

Set 5: As a variant to Set 4 the nucleosides in position 14 can be ribose or HM and optionally the nucleoside in position 16 can also be ribose or HM.

Nuclease activity against oligo drugs can vary substantially between species and between organ/tissue/cell types. Further, many modifications, such as the phosphorothioate linkage, can inhibit RNAi activity depending on how many such linkages are present and where they are located in the strand. The patterns of resistant linkage just described, however, can be adjusted for use in subjects on an as needed basis for a particular species and particular cell types. Susceptible linkages sites in particular organ/tissue/cell types in any subject species can be determined using an established technique most notably LC-MS (liquid chromatography combined with mass spectrometry). Any linkage sites found to be preferentially cleaved by an endonuclease can be made more resistant by adding a more resistant linkage and/or a more resistant sugar modification to one or both or the nucleosides in any preferentially cleaved linkage site. The relative resistance of the linkages available for positions 2-19 is phosphodiester<phosphorothioate<boranophosphate<N3′ Phosphoramidate; while the relative resistance of the nucleoside sugars is ribose<2′-fluoro<2′-O-methyl<AGSD sugar modifications.

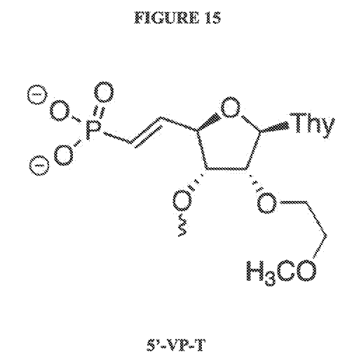

Any of the ags-siRNA and ags-IMiR compounds suitable for use in subjects without a protective carrier can be further modified by conjugating C16 (FIG. 14) to the nucleoside in position 1 or 8 counting from the 5′-end as described in Lima et al., Cell 150: 883-894, 2012 and/or Prakash et al., Nucleic Acids Res 43: 2993-3011, 2015.

### E. Designing AgsRNAi Compounds

### 5′-End Modifications

### 1. 5′-End Phosphates and Phosphate Analogs:

AgsRNAi compounds of the present invention can be manufactured with a hydroxyl, 5′-end phosphate group or a 5′-end phosphate analog on the 5′ carbon of the 5′-end nucleoside sugar. FIG. 12 illustrates a 5′-end phosphate group on a ribose sugar. The nucleoside sugars promoting nuclease resistance that can be used in position 1 of an agsRNAi compound are selected from the group consisting of 2′-fluoro, 2′-O-methyl, 2′-methoxyethyl, 2′-deoxyribose, LNA, FANA, 4'S-FANA, ALN, AENA, CENA, HM, HNA, EA, F-CeNA, CeNA, UNA, CRN R monomer and CRN Q monomer.

A number of linkages that are more nuclease resistant than phosphorothioates are provided herein. A subset of these can be used to link nucleoside positions 1 and 2. One advantage of this is that it allows ribose or 2′-fluoro to be used in position 1 in agsRNAi compounds that are not administered with a protective carrier. The advantage of having ribose or 2′-fluoro in position 1 is that a nucleoside with one of these sugars and a hydroxyl on the 5′ carbon of the 5′-end nucleoside sugar is more susceptible to the addition of a 5′-end phosphate group by an intracellular kinase compared to nucleosides with one of the other sugars. The linkage between positions 1-2 suitable for this purpose can be N3′ phosphoramidate or amide. These and other linkages suitable for use in oligonucleotides are reviewed in Deleavy and Damha (Chemistry & Biology 19: 937-53, 2012).

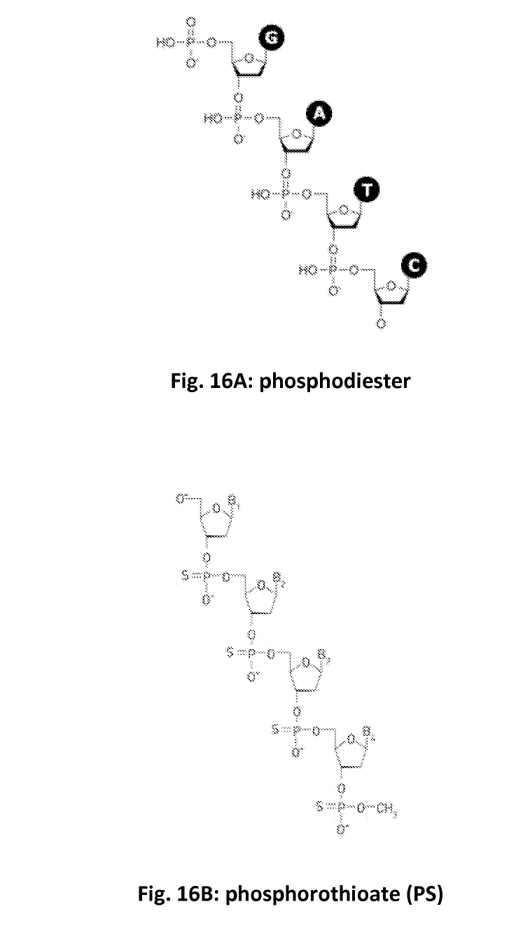

The 5′-phosphate analog suitable for conjugation some of these 5′-end nucleosides is 5′-(E)-vinylphosphonate (5′-VP). It is described along with synthesis methods in publications that include the following: Lima et al., Cell 150: 883-894, 2012; Prakash et al., Nucleic Acids Res 43: 2993-3011, 2015; WO 2011/139699; WO 2011/139702. 5′-VP is illustrated in FIG. 15 where it is conjugated to a thymidine with a 2′-methoxyethyl modification. It can be used with any of one of the nucleoside sugar chemistries provided herein where 2′-O-methyl, 2′-methoxyethyl, FANA, EA, AENA, CENA and HM are preferred.

The 5′-end nucleoside is not available to interact with the target so it can have any of standard bases (G, C, U and A). Consequently, this base can be selected on the basis of the base found on the nucleoside in position 2. As a result the 5′-terminal linkage site can be designed to have one of the 8 linkage sites that are generally more resistant to nuclease attack than the 8 other possible linkage site combinations that can be generated on the basis of different combinations of standard bases. Thus, depending on what base is in position 2 the base for the nucleoside in position 1 optionally can be selected from the group consisting of A-A, U-U, G-G, G-C, G-U, G-A, A-G and C-U.

### F. Designing AgsRNAi Compounds

### Nucleoside Positions 2-19

### 1. Avoiding Steric Hindrance Problems:

Steric hindrance problems related to modifications of guide strands have been identified in two different ways. One of these involves summarizing a number of studies where various modifications to guide strands in particular positions have been shown to reduce silencing activity where the reduction has been attributed to the modification projecting a moiety into either the major or minor grove of the duplex formed by the guide strand and the passenger strand. Even though agsRNAi compounds are typically used without a passenger strand nevertheless modifications to guide strands that can produce these steric hindrance problems should be avoided.

With respect to sugar modifications it is clear, for example, that 2′-methoxyethyl modifications in most guide strand positions in the range of positions 1-19 reduce silencing activity. The exceptions are position 1 and any one of the positions 17-19. The 2′-O-methyl modification, however, is tolerated individually in any one of the positions 1-19 (other than position 2 in the case of miRNA mimics such as ags-MiRs) so the size of this modification provides a convenient approximate limit on the size of any sugar moiety projections into the helical grove.

The other way of identifying potential steric hindrance problems based on moieties projecting from a modified sugar was based on noting that 2′-O-methyl in position 14 substantially inhibits silencing activity in both guide strands with siRNA and those with miRNA activity. To a lesser degree this is also true for nucleosides in position 16. It is now clear from detailed studies of the interaction of the various nucleosides and linkages in a guide strand with human AGO-2 that the nucleosides in positions 14-18 are threaded through a narrow channel between the PAZ and N domains (Schirle et al., Science 346: 608-613, 2014). The narrowness of this channel imposes limits on the size of the 2′ modifications that can be made to the sugars of the nucleosides passing through this channel. This limit is set by the size of the 2′-O-methyl group, which is approximately 23 cubic angstroms in size. So larger 2′-end modifications should not be used in positions 15, 17 and 18 of agsRNAi and smaller 2′ modifications such as fluoro, if any, should be used in positions 14 and 16.

There is less data of this type related to possible or actual steric hindrance problems generated by moieties projecting from nucleoside bases. But is clear from studies using the 5-methylcytosine base that the resulting projection of the methyl group into the major grove causes steric hindrance problems reflected in substantially reduced siRNA silencing activity when the base was in nucleoside position 10, 12 or 14 (Elmen et al., Nucleic Acids Res 33: 439-447, 2005). The 5-methylcytosine did not cause a reduction in silencing when it was in position 2, however. The Schirle et al. (2014) study indicates this base modification would also be tolerated in positions 3-6, 8, 9 and 17-19. Thus, 5-methylcytosine and other bases with a 23 cubic angstrom or larger moiety projecting from the 5 position of the base, such as the AGSD base 5-methyluracil are preferably not used in the positions where 5-methylcytosine has been shown to reduce silencing activity in double strand RNAi triggers.

Moieties on some other bases, such as those conjugated to the C-5 position of some pyrimidine bases, also can project in to the major groove in an A-type duplex. For use as bases in agsRNAi in nucleoside positions where 5-methylcytosine is acceptable these moieties are preferred to be smaller than a propynyl group. Propynl groups occupy approximately 53 cubic angstroms of space while the 2′-O-methyl group occupies approximately 23 cubic angstroms of space.

These findings set limits on the size of sugar and base modification moieties in one or more of positions 2-19 that would project into the major or minor groove in order to qualify them for possible use in an agsRNAi compound. More specifically, in the case of 2′ carbon accommodating modifications to ribose or to the corresponding carbon in another sugar it is preferred that the structure be no larger than a 2′-O-methyl. Such alkyl groups project into the minor groove that is narrower than the major groove. The exceptions to this rule include the 5′-end terminal nucleoside and nucleoside(s), if any, in the overhang precursor. Here larger 2′-modifications such as those projecting from 2′-methyoxyethyl or EA can be tolerated. Another exception is modifications to the nucleoside in the second position from the 5′-end of an ags-siRNA or ags-IMiR where a nucleoside modification of the type provided for herein and in the art that reduces off target effects due to the seed region acting as a targeting code can be used.

Sugar and base modifications that promote the C3′-endo conformation in the nucleoside in which they are found and in contiguous nucleosides with the most flexible and/or flexible sugars and that do not cause steric hindrance problems that significantly reduce silencing activity are suitable for use as AGSD modifications in agsRNAi compounds.

The existence of substantial steric hindrance problems that would disqualify a particular sugar or base modification from being an AGSD modification can be operationally identified. When present in a conventional double strand RNAi drug or trigger, AGSD moieties or modifications do not substantially interfere with the RNAi activity of the drug or trigger. Substantial interference in the case of a modification to a guide strand with siRNA activity being equal to or greater than a 20, 30, 40, 50, 60, 70 or 80% reduction in the level of target suppression at ED₅₀ dose seen for the cognate siRNA without the test modification.

Substantial interference in the case of a modification to a guide strand with miRNA activity being equal to or greater than a 20, 30, 40, 50, 60, 70 or 80% reduction in the level of suppression of 1, 2, 3 or 4 targets at ED₅₀ dose seen for the cognate miRNA without the test modification.

Since modifications to sugars and/or bases typically affect the regional intrastrand binding affinity between passenger and guide strands the insertion of such test modifications into the guide strand must be accompanied by any needed modifications to the passenger strand to ensure the efficient loading of the intended guide strand into RISC. In particular the affinities of the terminal 4 duplexed nucleosides at each end of the RNAi trigger must be consistent with the asymmetry rule that states the duplexed end with the 5′-end of the passenger strand must have a higher intrastrand affinity then the terminal duplex with the 5′-end of the guide strand.

### 2. AGSD Sugar Modifications:

AGSD sugar modifications suitable for use in the present invention can be any of those sugars that meet the criteria provided herein for selecting such sugars. The specific AGSD sugars provided herein for use in agsRNAi compounds in positions 2-19 are independently selected from the group consisting of LNA, HNA, ANA, CRN R monomer, CRN Q monomer, HM, FHNA, CeNA, F-CeNA and cEt.

AGSD sugar modifications vary in their flexibility. Flexibility is a measure of how readily the sugar assumes a conformation other than C3′-endo when it is a component of a nucleoside and is acted on by one or more contiguous nucleosides linked to it by a phosphodiester or phosphorothioate linkage and where the contiguous sugar(s) prefer a conformation other than C3′-endo. Examples of nucleotide sugars that prefer other conformations are 2′-deoxyribose and FANA.

When considering sugar conformations and the effects of continuous nucleoside sugars it is assumed the sugar in question is in a nucleoside that is linked to other nucleosides in a single stranded structure that is in solution under physiologic salt and pH conditions. The less flexible the AGSD sugar the less likely its conformation is to be affected by contiguous nucleosides but the more likely it is to influence the conformation of contiguous nucleosides with equally flexible or less flexible sugars.

Table 1 ranks semi-quantitatively by their flexibility those non-AGSD and AGSD sugar modifications specifically provided for herein for use in agsRNAi. The sugars are assumed to be part of a nucleoside that is linked to other nucleosides and evaluated under the conditions as just discussed. As the table shows, the non-AGSD sugars are the most flexible and, therefore, are the most susceptible of the listed sugars to having the probability that their sugar will be in the C3′-endo conformation increased by the presence of a contiguous nucleoside with an AGSD sugar.

In addition, the likelihood that one of the non-AGSD sugars will be in a C3′-endo conformation and the ease with which it responds to the influence of contiguous nucleosides will depend on whether it has a purine or a pyrimidine base. Nucleosides with one of these non-AGSD sugars and a purine base will be less likely to have their sugar in a C3′-endo conformation compared to a nucleoside with a pyrimidine base and the former will be less likely to be influenced by a contiguous nucleoside with a AGSD modification.

The resistance of the more flexible sugar in a nucleoside with a purine base to the influence of contiguous nucleosides increases when nucleosides with more flexible sugars and purine bases occur in purine rich regions. For example, a purine rich region in a strand with a concentration of purine nucleosides such as -AAUA- or -AACA-, can promote more of a B-type that is characterized by a C2′-endo sugar conformation or an unstacked local area in a helix rather than the A-type that is characterized by the C3′-endo sugar conformation that is preferred when the bases are pyrimidines.

As the flexibility of the sugar in a given nucleoside decreases the more likely it is to be in the C3′-endo conformation and the more likely it is to influence the conformation of equally or more flexible sugars in contiguous nucleosides. The two sugars, LNA and cET, with a rigid C3′-endo conformation, however, are not susceptible to the influence of contiguous nucleosides.

TABLE 1
Ranking of AGSD and Non-AGSD Sugars by Flexibility
and by the Relative Effect of a Nucleoside Containing a Particular
Sugar on the Conformation of the Same or Contiguous Nucleoside(s)

| | | Examples of AGSD Sugars | | |
|---|---|---|---|---|
| | | Flexible | | |
| | Non-AGSD | HNA | | |
| | Sugars | FHNA | Semi-Flexible | |
| | Most Flexible | ANA | CRN | |
| | 2′-Fluoro | CeNA | (R monomer) | Rigid |
| | 2′-O-Methyl | F-CeNA | CRN | LNA |
| Conditions | Ribose | HM | (Q monomer) | cEt |
| Susceptibility of the | Purine | ++ | + | − |
| Indicated Sugar to | Base ++ | | | |
| having its | to +++ | | | |
| Conformation | Pyrimidine | | | |
| Influenced by a Less | Base ++++ | | | |
| Flexible Sugar in a | | | | |
| Contiguous | | | | |
| Nucleoside | | | | |
| Effectiveness of | N/A | ++ | +++ | ++++ |
| Particular | | | | |
| AGSD Sugars in | | | | |
| Affecting the | | | | |
| Conformation of | | | | |
| more flexible Sugars | | | | |
| Appearing in | | | | |
| Contiguous | | | | |
| Nucleosides | | | | |
| Probability the | N/A | +++ | ++++ | +++++ |
| AGSD Sugar will | | | | |
| Assume a C3′-endo | | | | |
| Conformation | | | | |
| When Part of a | | | | |
| Nucleoside | | | | |

These general considerations make it clear that the minimum frequency of AGSD sugar modifications needed to promote the activity, potency and duration of effect of agsRNAi over the ssRNAi known in the art will be based on a number of factors including the flexibility of the AGSD sugar and the flexibility of the contiguous sugars which will depend on whether they have purine or pyrimidine bases. It is also important to note that at most the influence of a AGSD sugar on contiguous more flexible sugars will extend about 3 nucleoside positions in one or both directions (i.e. upstream or downstream).

With respect to the density of AGSD sugar modifications it is important to note that AGSD sugars do not exactly match native ribose in a C3′-endo conformation. Since such deviations are multiplied when they appear in multiple contiguous nucleosides it is important to set upper limits on the frequency of these modifications in an agsRNAi strand.

The rules for applying AGSD sugar modifications to positions 2-19 of agsRNAi compounds are as follows:
1) AGSD sugar modifications to the seed sequence nucleosides of ags-MiRs that have the seed sequence as their targeting code are optional.
2) It is preferred that the sugar in position 2 of an ags-siRNA or an ags-IMiR be a 2′-O-methyl.
3) It is preferred that the sugar in position 2 of an ags-MiR be 2′-fluoro, ribose or HM.
4) In a purine rich region it is preferred that there be at least one nucleoside with an AGSD sugar modification per 3 nucleosides with a purine base.
5) It is preferred that there be no more than 5 contiguous nucleosides with a pyrimidine base without at least one nucleoside with an AGSD sugar and/or AGSD base.
6) It is preferred that there be at least one nucleoside with a non-AGSD sugar be between nucleosides with AGSD sugars.
7) It is preferred that there be at least 4 nucleosides with an AGSD sugar among nucleosides 2-19 and that at least 2 of these be in the 5′-end half of an ags-siRNA or an ags-IMiR and 2 in the 3′-end half
8) It is preferred that at least 2 nucleosides with an AGSD sugar be included in the 3′-end half of an ags-MiR where position 10 is considered to be in the 3′-end half.

### 3. AGSD Base Modifications:

AGSD base modifications suitable for use in the present invention can be any of those bases that meet the criteria provided herein for selecting such bases. The specific AGSD bases provided herein for use in agsRNAi compounds are independently selected from the group consisting of 2-thiouracil (U), 4-thiouracil (C), pseudouracil (U) and 5-methyluracil (U). The letter in parenthesis after the name of the base indicates which standard base the AGSD base substitutes for.

When pseudouracil is used in a nucleoside its ability to promote a C3′-endo conformation in a contiguous nucleoside with a more flexible or flexible sugars shown in Table 1 is asymmetric. It has a more potent effect on the sugar conformation of upstream nucleosides than on the downstream ones, for example, it can affect the sugar conformation in 2 or 3 upstream purine nucleosides with flexible sugars while only affecting one down stream purine nucleoside with a flexible sugar.

The rules for applying AGSD sugar modifications to positions 2-19 of agsRNAi compounds are as follows:
1) AGSD bases can be used in the same nucleoside with any of the non-AGSD and AGSD sugars provided herein.
2) It is preferred that there be at least one nucleoside with an AGSD base in any region having at least 5 consecutive pyrimidines containing nucleosides.
3) It is preferred that any purine rich region that is interrupted by a pyrimidine containing nucleoside that that nucleoside have an AGSD base.
4) 5-methyluracil is preferably not used in nucleoside positions 12, 14 or 16 of any agsRNAi and also preferably not used in positions 10 and 11 of an ags-siRNA or ags-IMiR.

### 4. Reducing Off-Target Effects:

The principal off-target effects caused by RNAi triggers generally and also by agsRNAi compounds involves either or both of the following: (1) the seed sequence of an ags-siRNA or ags-IMiR engaging targets and thus causing an unintended miRNA mimicking effect; and/or (2) the compound activates an innate immune response in a subject that leads to an unacceptable level symptoms.

Off-target effects due to the seed region directing an ags-siRNA or ags-IMiR to have unintended miRNA mimicking activity can be inhibited by using one or two of the following modifications as needed: (1) a 2′-O-methyl in position 2; (2) 1-3 CENA modified nucleosides inserted in the seed sequence with inclusion of position 3 from the 5′-end of the strand being preferred; (3) an UNA used preferably in position 7 from the 5′-end of the strand; and (4) replacing any adenine containing nucleoside with a modification selected from the group N2-propyl-2-aminopurine or N2-cyclopentyl-2-aminopurine and/or a replacing a guanine containing nucleoside with a N2-cyclopentylguanine modification where one or more of these modifications can be used in position(s) 2 and/or 7 from the 5′-end of the strand. These base modifications can be used with any of the sugar modifications provided herein.

Several of the modifications commonly applied to agsRNAi compounds will have the additional effect of reducing the likelihood the compound will engender an innate immune response. Any further inhibition of an innate immune response that might be needed can be achieved by using the 2′-O-methyl modification in one or more uracil, adenine or guanine containing nucleosides. It is preferred, however, that a 2′-O-methyl not be used in positions 14 or 16 in any agsRNAi compound and that it not be used in position 2 in an ags-MiR. 5-methylcytosine can also be used, as needed, to reduce innate immune responses but 5-methyluracil is preferably not used in nucleoside positions 12, 14 or 16 of any agsRNAi and also preferably not used in positions 10 and 11 of an ags-siRNA or ags-IMiR.

Off-target effects due to AGO-2 based catalytic silencing activity on the part of an ags-MiR with a seed sequence targeting code can be inhibited by replacing the nucleosides in positions 10 and/or 11 with one or two mismatches with the unintended target and/or replacing them with modified nucleosides with a base or sugar modification selected from the group 2,4-dichlorobenzene, 3-methyluracil, UNA, 5,6-dihydrouracil and N⁴,N⁴-dimethylcytosine. The modified bases can be used with any of the sugars or sugar substitutes provided herein. Alternatively, the modular approach can be used to generate an ags-MiR with the required 5′-end module along with a seed vehicle that does not recognize the unintended target.

### G. Designing Ags-MiR Compounds

### Using the Modular Approach

### 1. Overview:

In addition to modifying endogenous miRNA guide strands to generate ags-MiRs, ags-MiRs based on a seed sequence targeting code can be constructed by applying basic nuclease resistance, AGSD and other modifications provided herein to modular components that can be combined to form the active compound. This approach can increase the speed by which highly active ags-MiRs can be generated and can improve the silencing activity on the desired targets compared to ags-MiRs based on the entire sequence of any endogenous miRNA guide strand to be mimicked. The seed sequence used in an ags-MiR can be taken from an endogenous miRNA or be a novel sequence selected for its ability to direct the silencing a particular mRNA or set of mRNAs.

Moving in the 5′ to 3′ direction the modules are the following: (1) the 5′-end module that consists of the first 8 or 9 consecutive nucleosides and, therefore, includes the seed sequence; (2) the seed vehicle that includes the next 10 or 11 nucleosides depending on the length of the 5′-end module to bring the total length exclusive of any overhang precursor to 19; and (3) the overhang precursor if any.

### 2. 5′-End Module:

MiRbase (www.mirbase.org) provides the sequences for the 5′-end 8 or 9 nucleosides for endogenous human miRNAs that will be used to generate ags-MiR mimics of a particular naturally occurring miRNA. The base for the nucleoside in position 1, however, can be any of the standard bases provided herein (A, G, C, U and T). This nucleoside will be modified in accordance with the rules stated in the section entitled “Designing AgsRNAi Compounds: 5′-End Modifications.”

The rest of the nucleosides in the 5′-end module are then modified in accordance with one set of the rules for providing basic nuclease resistance. AGSD modifications are then added but are optional for the seed sequence. The reason these modifications are optional in the seed sequence is that AGSD modifications increase the binding affinity of the seed sequence for their target and thereby can increase the silencing activity. Thus, the use of AGSD modifications to this region or not will depend on the outcomes the ags-MiR is intended to achieve with respect to target suppression levels.

Table 2 provides a list of modifications that can be made to seed sequence nucleosides in order to increase their binding affinity with their targets. These include some non-AGSD and some AGSD modifications.

TABLE 2
EXAMPLES OF NUCLEOSIDE MODIFICATIONS
SUITABLE FOR USE IN THE SEED SEQUENCES
OF ags-MiR COMPOUNDS TO INCREASE THEIR
BINDING AFFINITY WITH THEIR TARGETS

| | Degrees | |
|---|---|---|
| | Change | |
| | in Tm per | |
| | Modifi- | |
| Modification | cation | Comments |
| 2′-fluoro | plus 1.0- | |
| | plus 2.0 | |
| 2′-O-methyl | plus 0.5- | |
| | plus 1.0 | |
| LNA | plus 2.0- | LNA with adenine has about one-half of |
| | plus 9.0 | the stabilizing effect of LNAs with other |
| | | standard bases. Using 2,6-diaminopurine |
| | | or replacing a complementary uracil |
| | | containing nucleoside with an LNA with a |
| | | thymine base can reverse this. Using a 2- |
| | | thiothymine replacement for a thymine can |
| | | increase the affinity of a LNA bringing it |
| | | to the upper end of the indicated Tm range. |
| 2,6- | plus 1.0- | Replacement of adenine with 2,6- |
| diaminopurine | plus 3.5 | diaminopurine increases the Tm. It can be |
| | | paired with any of the sugar modifications |
| | | or substitutes provided for herein to form a |
| | | nucleoside. The complementary partner |
| | | nucleoside can have uracil or thymine. |
| 2-thiouracil | plus 1.0- | The complimentary nucleoside in the |
| | plus 3.0 | target strand must contain adenine rather |
| | | than guanine when the goal is to optimize |
| | | stability. The most stabilizing nucleosides |
| | | have 2-thiouridine paired with LNA where |
| | | the use of this base further increases the |
| | | stabilizing effect of LNA. |
| 4-thiouracil | plus 3.0- | 4-thiouracil can be paired with any of the |
| | plus 6.0 | sugar modifications or sugar substitutes |
| | | provided for herein to form a nucleoside. |
| | | The complimentary nucleoside in the |
| | | partner strand must contain guanine rather |
| | | than adenine when the goal is to increase |
| | | stability particularly relative to a U:G |
| | | wobble base. The most stabilizing |
| | | nucleosides have 4-thiouracil paired with |
| | | LNA where the use of this base further |
| | | increases the stabilizing effect of the LNA |
| | | modification. |
| 5- | plus 1.0 | |
| methylcytosine | | |
| Pseudouracil | plus 1.0- | |
| | plus 2.0 | |
| 2-thiothymine | plus 2.0- | 2-thiothymine can be paired with any of |
| | plus 6.0 | the sugar modifications or sugar substitutes |
| | | provided for herein to form a nucleoside. |
| | | The most stabilizing nucleosides have 2- |
| | | thiothymine paired with LNA where the |
| | | use of this base further increases the |
| | | stabilizing effect of LNA. |

### 3. Seed Vehicle:

Any given seed vehicle sequence can be used with any given 5′-end module and these used with any overhang precursor that is provided herein. In principle a seed vehicle can have any nucleotide sequence but certain sequences and particular sets of AGSD modification to its nucleosides are more effective at promoting the activity, potency and/or duration of effect of the ags-MiR containing it than other seed vehicle sequences and sets of AGSD modification. Thus, it is important to use optimized seed vehicles for the production of ags-MiR compounds based on the modular approach.

Particular seed vehicle sequence are modified according to one of the sets of rules for providing basic nuclease resistance and then modified according to AGSD using the rules set out for nucleosides in positions 2-19. Thirty examples of unmodified 11-mer seed vehicle sequences are provided in Table 3 and examples of how seed vehicle sequence number 13 can be modified are provided in Table 4. Here basic nuclease resistance rules applied to the seed vehicle are suitable for an ags-MiR suitable for use with or without a protective carrier. Examples of 7 modified 10-mer seed vehicle sequences are provided in Table 19.

TABLE 3
EXAMPLES OF 11-mer SEED VEHICLE SEQUENCES

| Sequence | Sequence* | Seq ID |
|---|---|---|
| Number | 5′-3′ | No. |

| | |
|---|---|
| 1 | GUUCUUUCUUU |
| 2 | AGCUUCUUCUU |
| 3 | GGUCUUAUUUG |
| 4 | GGUGUUCAUUU |
| 5 | UUCUUUCUUUU |
| 6 | GCGCUUCUCUU |
| 7 | UUUUUUCUCUU |
| 8 | GUUUUCUUUCU |
| 9 | GUUCUCUUCUC |
| 10 | CUUUUGUUCUC |
| 11 | CUUCUUCUUUC |
| 12 | GUUCUUGUUUC |
| 13 | CGGUUUCGUUU |
| 14 | GCGCCUUUCUU |
| 15 | GUCCUUGCUUC |
| 16 | CUUCCUUCUUC |
| 17 | GCCUCUUUUCC |
| 18 | GCGUUCCUUCU |
| 19 | GGGUCGUUUUU |
| 20 | GCGCCGAUUUU |
| 21 | GCUCCCUUUCU |
| 22 | AGUCUUUUCUU |
| 23 | AGGCUUUCUUU |
| 24 | GCGCUCUCUUC |
| 25 | GUUUCGUUCGA |
| 26 | GUUGCGUUCUA |
| 27 | GUUCUCGUCUC |
| 28 | GGCCGACCUAC |
| 29 | GUCCCUUCUUC |
| 30 | GCCCUCCCUGA |

Any of the nucleosides in Table 4 with an L modification(s) found in one or more of positions 9-19 (based on full length ags-MiR) can have any given L or set of Ls replaced with any of those sugars provided for herein for positions 2-19 including one or more of them being independently selected from the group consisting of: S, J, W, V, Q, Y, O, T and cet (i.e., if multiple Ls are replaced the selected replacements do not all have to be the same sugar). The key for the modifications is found in the section just before Table 6 that is entitled: “Nucleoside and Linkage Modification Key for Examples of agsRNAi Compounds Found in Tables 6-21.”

TABLE 4
EXAMPLES OF MODIFIED 11-mer SEED VEHICLE NUMBER 1

~C_{M4}G_{F}~G_{M}U_{F3}~U_{M}U_{F}~C_{M}G_{F}~U_{M}~U_{F3}~U_{M}
~C_{M}G_{F}~G_{M}U_{F}~U_{M3}U_{F}~C_{M}G_{F}~U_{M3}~U_{F}~U_{M3}
~C_{M}G_{F}~G_{M}U_{F3}~U_{M3}U_{F}~C_{M}G_{F}~U_{M3}~U_{F3}~U_{M}
~C_{M4}G_{F}~G_{M}U_{F3}~U_{M}U_{F}~C_{M4}G_{F}~U_{M3}~U_{F3}~U_{M}
~C_{M4}G_{F}~G_{M}U_{F12}~U_{M}U_{F}~C_{M}G_{F}~U_{M}~U_{F12}~U_{M}
~C_{M}G_{F}~G_{M}U_{F}~U_{M12}U_{F}~C_{M}G_{F}~U_{M12}~U_{F}~U_{M12}
~C_{M}G_{F}~G_{M}U_{F12}~U_{M12}U_{F}~C_{M}G_{F}~U_{M12}~U_{F12}~U_{M}
~C_{M4}G_{F}~G_{M}U_{F12}~U_{M}U_{F}~C_{M4}G_{F}~U_{M12}~U_{F12}~U_{M}
~C_{M4}G_{F}~G_{M}U_{F}~U_{M}U_{F}~C_{M}G_{F}~U_{M}~U_{F18}~U_{M}
~C_{M}G_{F}~G_{M}U_{F}~U_{M18}U_{F}~C_{M}G_{F}~U_{M18}~U_{F}~U_{M18}
~C_{M}G_{F}~G_{M}U_{F}~U_{M18}U_{F}~C_{M}G_{F}~U_{M18}~U_{F18}~U_{M}
~C_{M4}G_{F}~G_{M}U_{F}~U_{M}U_{F}~C_{M4}G_{F}~U_{M18}~U_{F18}~U_{M}
~C_{M4}G_{F}~G_{M}U_{F3}~U_{M}U~C_{M}G_{F}~U_{M}~U_{F3}~U_{M}
~C_{M}G_{F}~G_{M}U_{F}~U_{M3}U~C_{M}G_{F}~U_{M3}~U_{F}~U_{M3}
~C_{M}G_{F}~G_{M}U_{F3}~U_{M3}U~C_{M}G_{F}~U_{M3}~U_{F3}~U_{M}
~C_{M4}G_{F}~G_{M}U_{F3}~U_{M}U~C_{M4}G_{F}~U_{M3}~U_{F3}~U_{M}
~C_{M4}G_{F}~G_{M}U_{F12}~U_{M}U~C_{M}G_{F}~U_{M}~U_{F12}~U_{M}
~C_{M}G_{F}~G_{M}U_{F}~U_{M12}U~C_{M}G_{F}~U_{M12}~U_{F}~U_{M12}
~C_{M}G_{F}~G_{M}U_{F12}~U_{M12}U~C_{M}G_{F}~U_{M12}~U_{F12}~U_{M}
~C_{M4}G_{F}~G_{M}U_{F12}~U_{M}U~C_{M4}G_{F}~U_{M12}~U_{F12}~U_{M}
~C_{M4}G_{F}~G_{M}U_{F}~U_{M}U~C_{M}G_{F}~U_{M}~U_{F18}~U_{M}
~C_{M}G_{F}~G_{M}U_{F}~U_{M18}U~C_{M}G_{F}~U_{M18}~U_{F}~U_{M18}
~C_{M}G_{F}~G_{M}U_{F}~U_{M18}U~C_{M}G_{F}~U_{M18}~U_{F18}~U_{M}
~C_{M4}G_{F}~G_{M}U_{F}~U_{M}U~C_{M4}G_{F}~U_{M18}~U_{F18}~U_{M}
C_{M}G_{L}G_{M}U_{F}U_{M}U_{F}C_{M}G_{L}U_{M}U_{F}U_{M}
C_{M}G_{L}G_{M}U_{F}U_{M}UC_{M}G_{L}U_{M}U_{F}U_{M}
~C_{M4}G_{L}~G_{M}U_{F3}~U_{M}U_{F}~C_{M}G_{L}~U_{M}~U_{F3}~U_{M}
~C_{M}G_{L}~G_{M}U_{F}~U_{M3}U_{F}~C_{M}G_{L}~U_{M3}~U_{F}~U_{M3}
~C_{M}G_{L}~G_{M}U_{F3}~U_{M3}U_{F}~C_{M}G_{L}~U_{M3}~U_{F3}~U_{M}
~C_{M4}G_{L}~G_{M}U_{F3}~U_{M}U_{F}~C_{M4}G_{L}~U_{M3}~U_{F3}~U_{M}
~C_{M4}G_{L}~G_{M}U_{F12}~U_{M}U_{F}~C_{M}G_{L}~U_{M}~U_{F12}~U_{M}
~C_{M}G_{L}~G_{M}U_{F}~U_{M12}U_{F}~C_{M}G_{L}~U_{M12}~U_{F}~U_{M12}
~C_{M}G_{L}~G_{M}U_{F12}~U_{M12}U_{F}~C_{M}G_{L}~U_{M12}~U_{F12}~U_{M}
~C_{M4}G_{L}~G_{M}U_{F12}~U_{M}U_{F}~C_{M4}G_{L}~U_{M12}~U_{F12}~U_{M}
~C_{M4}G_{L}~G_{M}U_{F}~U_{M}U_{F}~C_{M}G_{L}~U_{M}~U_{F18}~U_{M}
~C_{M}G_{L}~G_{M}U_{F}~U_{M18}U_{F}~C_{M}G_{L}~U_{M18}~U_{F}~U_{M18}
~C_{M}G_{L}~G_{M}U_{F}~U_{M18}U_{F}~C_{M}G_{L}~U_{M18}~U_{F18}~U_{M}
~C_{M4}G_{L}~G_{M}U_{F}~U_{M}U_{F}~C_{M4}G_{L}~U_{M18}~U_{F18}~U_{M}
~C_{M4}G_{L}~G_{M}U_{F3}~U_{M}U~C_{M}G_{L}~U_{M}~U_{F3}~U_{M}
~C_{M}G_{L}~G_{M}U_{F}~U_{M3}U~C_{M}G_{L}~U_{M3}~U_{F}~U_{M3}
~C_{M}G_{L}~G_{M}U_{F3}~U_{M3}U~C_{M}G_{L}~U_{M3}~U_{F3}~U_{M}
~C_{M4}G_{L}~G_{M}U_{F3}~U_{M}U~C_{M4}G_{L}~U_{M3}~U_{F3}~U_{M}
~C_{M4}G_{L}~G_{M}U_{F12}~U_{M}U~C_{M}G_{L}~U_{M}~U_{F12}~U_{M}
~C_{M}G_{L}~G_{M}U_{F}~U_{M12}U~C_{M}G_{L}~U_{M12}~U_{F}~U_{M12}
~C_{M}G_{L}~G_{M}U_{F12}~U_{M12}U~C_{M}G_{L}~U_{M12}~U_{F12}~U_{M}
~C_{M4}G_{L}~G_{M}U_{F12}~U_{M}U~C_{M4}G_{L}~U_{M12}~U_{F12}~U_{M}
~C_{M4}G_{L}~G_{M}U_{F}~U_{M}U~C_{M}G_{L}~U_{M}~U_{F18}~U_{M}
~C_{M}G_{L}~G_{M}U_{F}~U_{M18}U~C_{M}G_{L}~U_{M18}~U_{F}~U_{M18}
~C_{M}G_{L}~G_{M}U_{F}~U_{M18}U~C_{M}G_{L}~U_{M18}~U_{F18}~U_{M}
~C_{M4}G_{L}~G_{M}U_{F}~U_{M}U~C_{M4}G_{L}~U_{M18}~U_{F18}~U_{M}
C_{M}G_{F}G_{L}U_{F}U_{M}U_{F}C_{L}G_{F}U_{L}U_{F}U_{M}
C_{M}G_{F}G_{L}U_{F}U_{M}UC_{L}G_{F}U_{L}U_{F}U_{M}
~C_{M4}G_{F}~G_{L}U_{F3}~U_{M}U_{F}~C_{L}G_{F}~U_{L}~U_{F3}~U_{M}
~C_{M}G_{F}~G_{L}U_{F}~U_{M3}U_{F}~C_{L}G_{F}~U_{L3}~U_{F}~U_{M3}
~C_{M}G_{F}~G_{L}U_{F3}~U_{M3}U_{F}~C_{L}G_{F}~U_{L3}~U_{F3}~U_{M}
~C_{M4}G_{F}~G_{L}U_{F3}~U_{M}U_{F}~C_{L4}G_{F}~U_{L3}~U_{F3}~U_{M}
~C_{M4}G_{F}~G_{L}U_{F12}~U_{M}U_{F}~C_{L}G_{F}~U_{L}~U_{F12}~U_{M}
~C_{M}G_{F}~G_{L}U_{F}~U_{M12}U_{F}~C_{L}G_{F}~U_{L12}~U_{F}~U_{M12}
~C_{M}G_{F}~G_{L}U_{F12}~U_{M12}U_{F}~C_{L}G_{F}~U_{L12}~U_{F12}~U_{M}
~C_{M4}G_{F}~G_{L}U_{F12}~U_{M}U_{F}~C_{L4}G_{F}~U_{L12}~U_{F12}~U_{M}
~C_{M4}G_{F}~G_{L}U_{F}~U_{M}U_{F}~C_{L}G_{F}~U_{L}~U_{F18}~U_{M}
~C_{M}G_{F}~G_{L}U_{F}~U_{M18}U_{F}~C_{L}G_{F}~U_{L18}~U_{F}~U_{M18}
~C_{M}G_{F}~G_{L}U_{F}~U_{M18}U_{F}~C_{L}G_{F}~U_{L18}~U_{F18}~U_{M}
~C_{M4}G_{F}~G_{L}U_{F}~U_{M}U_{F}~C_{L4}G_{F}~U_{L18}~U_{F18}~U_{M}
~C_{M4}G_{F}~G_{L}U_{F3}~U_{M}U~C_{L}G_{F}~U_{L}~U_{F3}~U_{M}
~C_{M}G_{F}~G_{L}U_{F}~U_{M3}U~C_{L}G_{F}~U_{L3}~U_{F}~U_{M3}
~C_{M}G_{F}~G_{L}U_{F3}~U_{M3}U~C_{L}G_{F}~U_{L3}~U_{F3}~U_{M}
~C_{M4}G_{F}~G_{L}U_{F3}~U_{M}U~C_{L4}G_{F}~U_{L3}~U_{F3}~U_{M}
~C_{M4}G_{F}~G_{L}U_{F12}~U_{M}U~C_{L}G_{F}~U_{L}~U_{F12}~U_{M}
~C_{M}G_{F}~G_{L}U_{F}~U_{M12}U~C_{L}G_{F}~U_{L12}~U_{F}~U_{M12}
~C_{M}G_{F}~G_{L}U_{F12}~U_{M12}U~C_{L}G_{F}~U_{L12}~U_{F12}~U_{M}
~C_{M4}G_{F}~G_{L}U_{F12}~U_{M}U~C_{L4}G_{F}~U_{L12}~U_{F12}~U_{M}
~C_{M4}G_{F}~G_{L}U_{F}~U_{M}U~C_{L}G_{F}~U_{L}~U_{F18}~U_{M}
~C_{M}G_{F}~G_{L}U_{F}~U_{M18}U~C_{L}G_{F}~U_{L18}~U_{F}~U_{M18}
~C_{M}G_{F}~G_{L}U_{F}~U_{M18}U~C_{L}G_{F}~U_{L18}~U_{F18}~U_{M}
~C_{M4}G_{F}~G_{L}U_{F}~U_{M}U~C_{L4}G_{F}~U_{L18}~U_{F18}~U_{M}
C_{M}G_{L}G_{M}U_{F}U_{L}U_{F}C_{M}G_{F}U_{M}U_{L}U_{M}
C_{M}G_{L}G_{M}U_{F}U_{L}UC_{M}G_{F}U_{M}U_{L}U_{M}
~C_{M4}G_{L}~G_{M}U_{F3}~U_{L}U_{F}~C_{M}G_{F}~U_{M}~U_{L3}~U_{M}
~C_{M}G_{L}~G_{M}U_{F}~U_{L3}U_{F}~C_{M}G_{F}~U_{M3}~U_{L}~U_{M3}
~C_{M}G_{L}~G_{M}U_{F3}~U_{L3}U_{F}~C_{M}G_{F}~U_{M3}~U_{L3}~U_{M}
~C_{M4}G_{L}~G_{M}U_{F3}~U_{L}U_{F}~C_{M4}G_{F}~U_{M3}~U_{L3}~U_{M}
~C_{M4}G_{L}~G_{M}U_{F12}~U_{L}U_{F}~C_{M}G_{F}~U_{M}~U_{L12}~U_{M}
~C_{M}G_{L}~G_{M}U_{F}~U_{L12}U_{F}~C_{M}G_{F}~U_{M12}~U_{L}~U_{M12}
~C_{M}G_{L}~G_{M}U_{F12}~U_{L12}U_{F}~C_{M}G_{F}~U_{M12}~U_{L12}~U_{M}
~C_{M4}G_{L}~G_{M}U_{F12}~U_{L}U_{F}~C_{M4}G_{F}~U_{M12}~U_{L12}~U_{M}
~C_{M4}G_{L}~G_{M}U_{F}~U_{L}U_{F}~C_{M}G_{F}~U_{M}~U_{L18}~U_{M}
~C_{M}G_{L}~G_{M}U_{F}~U_{L18}U_{F}~C_{M}G_{F}~U_{M18}~U_{L}~U_{M18}
~C_{M}G_{L}~G_{M}U_{F}~U_{L18}U_{F}~C_{M}G_{F}~U_{M18}~U_{L18}~U_{M}
~C_{M4}G_{L}~G_{M}U_{F}~U_{L}U_{F}~C_{M4}G_{F}~U_{M18}~U_{L18}~U_{M}
~C_{M4}G_{L}~G_{M}U_{F3}~U_{L}U~C_{M}G_{F}~U_{M}~U_{L3}~U_{M}
~C_{M}G_{L}~G_{M}U_{F}~U_{L3}U~C_{M}G_{F}~U_{M3}~U_{L}~U_{M3}
~C_{M}G_{L}~G_{M}U_{F3}~U_{L3}U~C_{M}G_{F}~U_{M3}~U_{L3}~U_{M}
~C_{M4}G_{L}~G_{M}U_{F3}~U_{L}U~C_{M4}G_{F}~U_{M3}~U_{L3}~U_{M}
~C_{M4}G_{L}~G_{M}U_{F12}~U_{L}U~C_{M}G_{F}~U_{M}~U_{L12}~U_{M}
~C_{M}G_{L}~G_{M}U_{F}~U_{L12}U~C_{M}G_{F}~U_{M12}~U_{L}~U_{M12}
~C_{M}G_{L}~G_{M}U_{F12}~U_{L12}U~C_{M}G_{F}~U_{M12}~U_{L12}~U_{M}
~C_{M4}G_{L}~G_{M}U_{F12}~U_{L}U~C_{M4}G_{F}~U_{M12}~U_{L12}~U_{M}
~C_{M4}G_{L}~G_{M}U_{F}~U_{L}U~C_{M}G_{F}~U_{M}~U_{L18}~U_{M}
~C_{M}G_{L}~G_{M}U_{F}~U_{L18}U~C_{M}G_{F}~U_{M18}~U_{L}~U_{M18}
~C_{M}G_{L}~G_{M}U_{F}~U_{L18}U~C_{M}G_{F}~U_{M18}~U_{L18}~U_{M}
~C_{M4}G_{L}~G_{M}U_{F}~U_{L}U~C_{M4}G_{F}~U_{M18}~U_{L18}~U_{M}
C_{L}G_{F}G_{M}U_{L}U_{M}U_{F}C_{M}G_{F}U_{L}U_{F}U_{L}
C_{L}G_{F}G_{M}U_{L}U_{M}UC_{M}G_{F}U_{L}U_{F}U_{L}
~C_{L4}G_{F}~G_{M}U_{L3}~U_{M}U_{F}~C_{M}G_{F}~U_{L}~U_{F3}~U_{L}
~C_{L}G_{F}~G_{M}U_{L}~U_{M3}U_{F}~C_{M}G_{F}~U_{L3}~U_{F}~U_{L3}
~C_{L}G_{F}~G_{M}U_{L3}~U_{M3}U_{F}~C_{M}G_{F}~U_{L3}~U_{F3}~U_{L}
~C_{L4}G_{F}~G_{M}U_{L3}~U_{M}U_{F}~C_{M4}G_{F}~U_{L3}~U_{F3}~U_{L}
~C_{L4}G_{F}~G_{M}U_{L12}~U_{M}U_{F}~C_{M}G_{F}~U_{L}~U_{F12}~U_{L}
~C_{L}G_{F}~G_{M}U_{L}~U_{M12}U_{F}~C_{M}G_{F}~U_{L12}~U_{F}~U_{L12}
~C_{L}G_{F}~G_{M}U_{L12}~U_{M12}U_{F}~C_{M}G_{F}~U_{L12}~U_{F12}~U_{L}
~C_{L4}G_{F}~G_{M}U_{L12}~U_{M}U_{F}~C_{M4}G_{F}~U_{L12}~U_{F12}~U_{L}
~C_{L4}G_{F}~G_{M}U_{L}~U_{M}U_{F}~C_{M}G_{F}~U_{L}~U_{F18}~U_{L}
~C_{L}G_{F}~G_{M}U_{L}~U_{M18}U_{F}~C_{M}G_{F}~U_{L18}~U_{F}~U_{L18}
~C_{L}G_{F}~G_{M}U_{L}~U_{M18}U_{F}~C_{M}G_{F}~U_{L18}~U_{F18}~U_{L}
~C_{L4}G_{F}~G_{M}U_{L}~U_{M}U_{F}~C_{M4}G_{F}~U_{L18}~U_{F18}~U_{L}
~C_{L4}G_{F}~G_{M}U_{L3}~U_{M}U~C_{M}G_{F}~U_{L}~U_{F3}~U_{L}
~C_{L}G_{F}~G_{M}U_{L}~U_{M3}U~C_{M}G_{F}~U_{L3}~U_{F}~U_{L3}
~C_{L}G_{F}~G_{M}U_{L3}~U_{M3}U~C_{M}G_{F}~U_{L3}~U_{F3}~U_{L}
~C_{L4}G_{F}~G_{M}U_{L3}~U_{M}U~C_{M4}G_{F}~U_{L3}~U_{F3}~U_{L}
~C_{L4}G_{F}~G_{M}U_{L12}~U_{M}U~C_{M}G_{F}~U_{L}~U_{F12}~U_{L}
~C_{L}G_{F}~G_{M}U_{L}~U_{M12}U~C_{M}G_{F}~U_{L12}~U_{F}~U_{L12}
~C_{L}G_{F}~G_{M}U_{L12}~U_{M12}U~C_{M}G_{F}~U_{L12}~U_{F12}~U_{L}
~C_{L4}G_{F}~G_{M}U_{L12}~U_{M}U~C_{M4}G_{F}~U_{L12}~U_{F12}~U_{L}
~C_{L4}G_{F}~G_{M}U_{L}~U_{M}U~C_{M}G_{F}~U_{L}~U_{F18}~U_{L}
~C_{L}G_{F}~G_{M}U_{L}~U_{M18}U~C_{M}G_{F}~U_{L18}~U_{F}~U_{L18}
~C_{L}G_{F}~G_{M}U_{L}~U_{M18}U~C_{M}G_{F}~U_{L18}~U_{F18}~U_{L}
~C_{L4}G_{F}~G_{M}U_{L}~U_{M}U~C_{M4}G_{F}~U_{L18}~U_{F18}~U_{L}
C_{M}G_{L}G_{M}U_{F}U_{L}U_{F}C_{M}G_{L}U_{M}U_{L}U_{M}
C_{M}G_{L}G_{M}U_{F}U_{L}UC_{M}G_{L}U_{M}U_{L}U_{M}
~C_{M4}G_{L}~G_{M}U_{F3}~U_{L}U_{F}~C_{M}G_{L}~U_{M}~U_{L3}~U_{M}
~C_{M}G_{L}~G_{M}U_{F}~U_{L3}U_{F}~C_{M}G_{L}~U_{M3}~U_{L}~U_{M3}
~C_{M}G_{L}~G_{M}U_{F3}~U_{L3}U_{F}~C_{M}G_{L}~U_{M3}~U_{L3}~U_{M}
~C_{M4}G_{L}~G_{M}U_{F3}~U_{L}U_{F}~C_{M4}G_{L}~U_{M3}~U_{L3}~U_{M}
~C_{M4}G_{L}~G_{M}U_{F12}~U_{L}U_{F}~C_{M}G_{L}~U_{M}~U_{L12}~U_{M}
~C_{M}G_{L}~G_{M}U_{F}~U_{L12}U_{F}~C_{M}G_{L}~U_{M12}~U_{L}~U_{M12}
~C_{M}G_{L}~G_{M}U_{F12}~U_{L12}U_{F}~C_{M}G_{L}~U_{M12}~U_{L12}~U_{M}
~C_{M4}G_{L}~G_{M}U_{F12}~U_{L}U_{F}~C_{M4}G_{L}~U_{M12}~U_{L12}~U_{M}
~C_{M4}G_{L}~G_{M}U_{F}~U_{L}U_{F}~C_{M}G_{L}~U_{M}~U_{L18}~U_{M}
~C_{M}G_{L}~G_{M}U_{F}~U_{L18}U_{F}~C_{M}G_{L}~U_{M18}~U_{L}~U_{M18}
~C_{M}G_{L}~G_{M}U_{F}~U_{L18}U_{F}~C_{M}G_{L}~U_{M18}~U_{L18}~U_{M}
~C_{M4}G_{L}~G_{M}U_{F}~U_{L}U_{F}~C_{M4}G_{L}~U_{M18}~U_{L18}~U_{M}
~C_{M4}G_{L}~G_{M}U_{F3}~U_{L}U~C_{M}G_{L}~U_{M}~U_{L3}~U_{M}
~C_{M}G_{L}~G_{M}U_{F}~U_{L3}U~C_{M}G_{L}~U_{M3}~U_{L}~U_{M3}
~C_{M}G_{L}~G_{M}U_{F3}~U_{L3}U~C_{M}G_{L}~U_{M3}~U_{L3}~U_{M}
~C_{M4}G_{L}~G_{M}U_{F3}~U_{L}U~C_{M4}G_{L}~U_{M3}~U_{L3}~U_{M}
~C_{M4}G_{L}~G_{M}U_{F12}~U_{L}U~C_{M}G_{L}~U_{M}~U_{L12}~U_{M}
~C_{M}G_{L}~G_{M}U_{F}~U_{L12}U~C_{M}G_{L}~U_{M12}~U_{L}~U_{M12}
~C_{M}G_{L}~G_{M}U_{F12}~U_{L12}U~C_{M}G_{L}~U_{M12}~U_{L12}~U_{M}
~C_{M4}G_{L}~G_{M}U_{F12}~U_{L}U~C_{M4}G_{L}~U_{M12}~U_{L12}~U_{M}
~C_{M4}G_{L}~G_{M}U_{F}~U_{L}U~C_{M}G_{L}~U_{M}~U_{L18}~U_{M}
~C_{M}G_{L}~G_{M}U_{F}~U_{L18}U~C_{M}G_{L}~U_{M18}~U_{L}~U_{M18}
~C_{M}G_{L}~G_{M}U_{F}~U_{L18}U~C_{M}G_{L}~U_{M18}~U_{L18}~U_{M}
~C_{M4}G_{L}~G_{M}U_{F}~U_{L}U~C_{M4}G_{L}~U_{M18}~U_{L18}~U_{M}

### H. Designing AgsRNAi Compounds

### Overhang Precursors

A wide range of overhang structures have been described for use at the 3′-end of passenger and/or guide strands of double strand RNAi triggers. Most often they comprise nucleosides with one of the commonly used bases (A, G, U, T, C) combined with one of the commonly used sugars (ribose, 2′-fluoro, 2′-O-methyl and 2′-methoxyethyl) and joined by a phosphodiester or phosphorothioate linkages. Usually they are 2 nucleosides in length. In addition, more nuclease resistant linkages have been used to link both nucleoside and certain non-nucleoside structures to generate overhangs suitable for use in double strand RNAi triggers. Since the linked structures are not necessarily nucleosides the term “unit” is used generally herein to refer to the linked structures in overhang precursors.

In principle any of these overhangs that are suitable for use in the guide strands of double strand RNAi triggers can be used as overhang precursors for agsRNAi but those with higher affinity for the PAZ domain of dicer and the argonaute proteins are preferred. In addition, three nucleoside long overhangs are preferred in agsRNAi compounds.

When nucleosides with bases capable of base pairing with the commonly used based appear in overhang precursors it is important to check to see if they promote a thermodynamically stable hairpin (50% or greater of the agsRNAi in the hairpin conformation under physiologic salt, pH and oligo concentration in the 10 nM or less range) in the agsRNAi. If such a hairpin is formed then a different sequence for the overhang nucleosides is selected.

Table 5 provides examples of 3 nucleoside long overhang precursors that are suitable for use in agsRNAi compounds. They are shown with phosphodiester (no linkage represented) or with phosphorothioate linkages (˜). One or more of the phosphorothioate linkages can be replaced with one of the other nuclease resistant linkages provided herein. The subscript L after a nucleoside in the table represents that the nucleoside has an LNA sugar and the subscript X represents a nucleoside with a ribose, 2′-fluoro, or 2′-O-methylsugar. Any of the nucleosides in Table 5 with an L modification can have the L modification replaced with one or two of the sugars independently selected from the group consisting of: F, J, W, V, Y, T, TL and I. The use of some of these structures as 3′-end guide strand overhangs in siRNA compounds have been described previously (Bramsen et al., Nucleic Acids Res 37: 2867-81, 2009).

TABLE 5
EXAMPLES OF THREE NUCLEOSIDE LONG
OVERHANG PRECURSORS FOR USE IN agsRNAi COMPOUNDS

Sequence 5′-3′

| Sequence | Phosphodiester | Phosphorothioate |
|---|---|---|
| Number | Linkages | Linkages |
| 1 | G_{L}U_{L}U_{X} | ~G_{L}~U_{L}~U_{X} |
| 2 | U_{L}U_{L}U_{X} | ~U_{L}~U_{L}~U_{X} |
| 3 | C_{L}U_{L}U_{X} | ~C_{L}~U_{L}~U_{X} |
| 4 | G_{L}C_{L}U_{X} | ~G_{L}~C_{L}~U_{X} |
| 5 | A_{L}G_{L}U_{X} | ~A_{L}~G_{L}~U_{X} |
| 6 | A_{L}G_{L}C_{X} | ~A_{L}~G_{L}~C_{X} |
| 7 | A_{L}A_{L}A_{X} | ~A_{L}~A_{L}~A_{X} |
| 8 | A_{L}G_{L}A_{X} | ~A_{L}~G_{L}~A_{X} |
| 9 | G_{L}A_{L}A_{X} | ~G_{L}~A_{L}~A_{X} |
| 10 | C_{L}G_{L}C_{X} | ~C_{L}~G_{L}~C_{X} |
| 11 | G_{L}G_{L}C_{X} | ~G_{L}~G_{L}~C_{X} |
| 12 | G_{L}G_{L}U_{X} | ~G_{L}~G_{L}~U_{X} |

Numerous 3′-end overhang precursor chemistries can promote activity and increase nuclease resistance. They can also be used in agsRNAi compounds. These include but are not limited to the following where the indicated nucleoside sugars can be used with any of the standard bases: (1) morpholino; (2) tricyclo-DNA (Ittig et al., Artif DNA, PNA & XNA 1: 9, 2010); (3) ribo-difluorotoluyl (Xia et al., ACS Chem Biol 1: 176, 2006); (4) 4′-thioribonucleotides (Hoshika et al., Chem Bio Chem 8: 2133, 2007); (5) 2′-O-methyl-4′-thioribonucleotide (Takahashi et al., Nucleic Acids Res 37: 1353, 2009; Matsuda, Yakugaku Zasshi 131: 285, 2011); (6) altritol-nucleoside (ANA) (Fisher et al., Nucleic Acids Res 35: 1064, 2007); (7) cyclohexenyl-nucleoside (CeNA) (Nauwelaerts et al., J Am Chem Soc 129; 9340, 2007; (8) piperazine (U.S. Pat. No. 6,841,675); (9) 5-bis(aminoethyl) aminoethylcarbamoylmethyl-2′-deoxyuridine or 5-bis(aminoethyl) aminoethylcarbamoylmethyl-thymidine (Masud et al., Bioorg Med Chem Lett 21: 715, 2010); and (10) peptide nucleic acid overhangs (Potenza et al., Int J Mol Sci 9: 299-315, 2008).

The nucleosides and some non-nucleoside units used in overhang precursors in agsRNAi strands can be linked together and to the nucleoside in position 19 to form 3′-end overhangs using a nuclease resistant linkage selected from the group phosphorothioate, phosphonoacetate, (PACE), thiophosphonoacetate (thio-PACE), methylborane phosphine, amide, carbamate, urea, thiourea, N3′phosphoramidate and amide. (Sheehan et al., Nucleic Acids Res 31: 4109, 2003; Krishna & Caruthers, J Amer Chem Soc 133: 9844, 2011; Iwase et al., Nucleic Acids Symposium Series 50: 175, 2006; Iwase et al., Nucleosides Nucleotides Nucleic Acids 26: 1451, 2007; Iwase et al., Nucleic Acids Symposium Series 53: 119, 2009; Ueno et al. Biochem Biophys Res Comm 330: 1168, 2005; Kitamura et al., Bioorganic & Medicinal Chemistry 21: 4494-501, 2013; Deleavy and Damha Chemistry & Biology 19: 937-53, 2012).

Further, 3′-end overhang precursors can be comprised of certain hydrophobic aromatic moieties. For example, those that are comprised of one to three units containing two six membered rings joined by phosphodiester, or one of the other linkages just listed where the unit(s) are attached to the agsRNAi nucleoside in position 19 by the same linkage and when multiple units are used they are also joined by the same linkage. Two unit structures are preferred in the following examples. Suitable ring structures include benzene, pyridine, morpholine and piperazine (U.S. Pat. No. 6,841,675). Structures based on the benzene and pyridine rings have been previously described for 3′-end overhang use in conventional siRNA by Ueno et al., Bioorg Med Chem Lett 18:194, 2008; Ueno et al., Bioorganic & Medicinal Chemistry 17: 1974, 2009; Kitamura et al., Bioorganic & Medicinal Chemistry 21: 4494-501, 2013. These units include 1,3-bis(hydroxymethyl)benzene, 1,3-bis(hydroxymethyl)pyridine and 1,2-bis(hydroxymethyl)benzene. These work particularly well with a linkage selected from the group carbamate, urea and thiourea. These structures are also suitable for agsRNAi use as overhang precursors.

In another example of possible non-nucleoside overhang precursors for agsRNAi include the aromatic moieties that can be biaryl units where the linkages holding the units together and to the oligo are covalently attached to benzene rings where the benzene ring is further covalently attached to a non-bridging moiety selected from the group benzene, naphthalene, phenanthrene, and pyrene (Ueno et al., Nucleic Acids Symposium Series 53: 27, 2009; Ueno et al., Bioorganic & Medicinal Chemistry 17: 1974-81. 2009; Yoshikawa et al., Bioconjugate Chem 22: 42, 2011).

In addition, the 3′-end overhangs, or lack thereof, can affect the intracellular distribution of agsRNAi. AgsRNAi compounds that diffuse into the nucleus can be expelled from the nucleus by Exportin-5 (Exp-5). This activity of Exp-5 can be rate-limiting for silencing activity at low doses of agsRNAi. Exp-5 binds to the first two nucleosides or units in any 3′-end overhang(s). Thus, agsRNAi designed with 3′-end overhang precursors comprising nucleosides have a potential advantage over agsRNAi compounds that do not have overhang precursors because they can produce a greater presence in the cytoplasm particularly at lower agsRNAi concentrations. For agsRNAi acting on nuclear targets, however, the lack of an overhang precursor or the presence of a non-nucleoside overhang precursor not engaged by Exp-5 can increase the activity of the compound by reducing its expulsion from the nucleus by Exp-5.

### I. Designing AgsRNAi Compounds: Additional Nuclease Resistant Linkages

### 1. Boranophosphate Linkage:

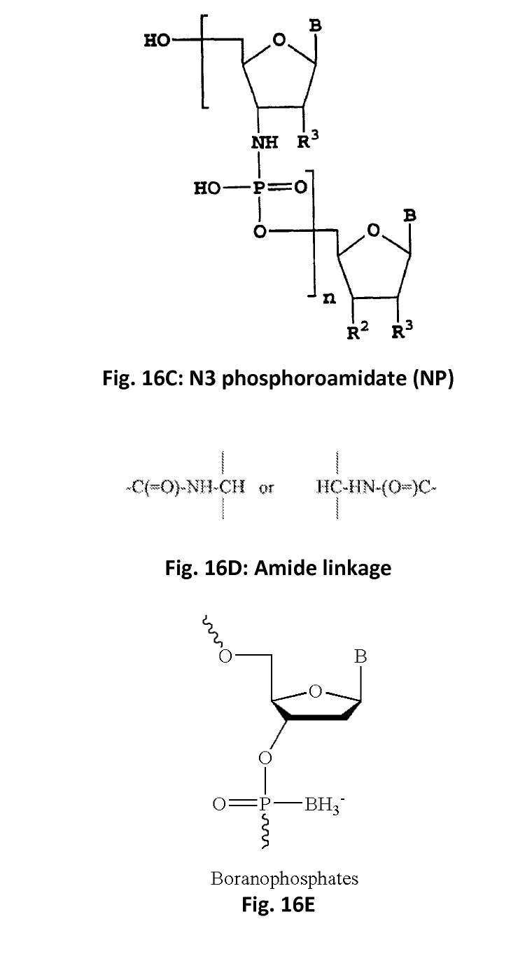

Selective use of the boranophosphate linkage (See FIG. 16E) in agsRNAi compounds can be used to increase their silencing activity. Linkage sites suitable for the use of the boranophosphate linkage include those involving nucleoside positions 4-5, 5-6, 6-7, 7-8 11-12 and to link nucleosides in an overhang precursor 2-4 units in length, ie., linkage sites 19-20, 20-21, 21-22 and 22-23. The presence of at least 3 boranophosphate linkages is preferred exclusive of the overhang precursor. The boranophosphate linkages can link nucleosides suitable for use in the region bracketed by nucleosides 2-19 and/or they can link nucleosides with a 2′-deoxyribose sugar that has been substituted for the sugar that would otherwise be present in the absence of the boranophosphate linkage. When a 2′-deoxyribose is used that has a pyrimidine base it is preferred that the base be replaced with the corresponding AGSD base.

A solid-phase synthesis method to produce oligos with boranophosphate linkages has recently been described, see FIGS. 17 and 18 (Uehara et al., J Organic Chem 79: 3465-72, 2014). This methodology simplifies the multiple earlier methods for incorporating the boranophosphate linkage into oligo nucleotides and it allow for the incorporation of a wider range of nucleoside sugars including LNA. Earlier methods include inserting boranophosphate linkages into oligos via one or the other of two general methods: (1) template directed enzymatic polymerization; and (2) chemical synthesis using solid supports. Boranophosphate oligo production can be achieved by a variety of solid phase chemical synthetic schemes including methods that involve modifications to the very commonly used approaches employing phosphoramidites or H-phosphonates in the production of phosphodiesters, phosphorothioates and phosphorodithioates among other chemistries (Li et al., Chem Rev 107: 4746, 2007). Other solid phase synthesis techniques more precisely directed to boranophosphates have also been developed. Wada and his colleagues, for example, have developed what they call the boranophosphotriester method that can make use of H-phosphonate intermediates (Shimizu et al., J Org Chem 71: 4262, 2006; Kawanaka et al., Bioorg Med Chem Lett 18: 3783, 2008). This method can also be applied to the synthesis of diastereometically pure boranophosphates (Enya et al., Bioorg Med Chem 16: 9154, 2008).

The generation of oligos with mixed linkages such as boranophosphate and a number of other linkages has been accomplished by several solid phase methods including one involving the use of bis(trimethylsiloxy)cyclododecyloxysilyl as the 5′-O-protecting group (Brummel and Caruthers, Tetrahedron Lett 43: 749, 2002). In another example the 5′-hydroxyl is initially protected with a benzhydroxybis(trimethylsilyloxy)silyl group and then deblocked by Et₃N:HF before the next cycle (McCuen et al., J Am Chem Soc 128: 8138, 2006). This method can result in a 99% coupling yield and can be applied to the synthesis of oligos with pure boranophosphate linkages or boranophosphate mixed with phosphodiester, phosphorothioate, phosphorodithioate or methyl phosphonate linkages. FIG. 16.

The boranophosphorylating reagent 2-(4-nitrophenyl)ethyl ester of boranophosphoramidate can be used to produce boranophosphate linked oligoribonucleosides (Lin, Synthesis and properties of new classes of boron-containing nucleic acids, PhD Dissertation, Duke University, Durham N.C., 2001). This reagent readily reacts with a hydroxyl group on the nucleosides in the presence of 1H-tetrazole as a catalyst. The 2-(4-nitrophenyl)ethyl group can be removed by 1,4-diazabicyclo[5.4.0]undec-7-ene (DBU) through beta-elimination, producing the corresponding nucleoside boranomonophosphates (NMPB) in good yield.

The stereo-controlled synthesis of oligo boranophosphates can be achieved using an adaptation of the oxathiaphospholane approach originally developed for the stereo-controlled synthesis of phosphorothioates (Li et al., Chem Rev 107: 4746, 2007). This method involves a tricoordinate phosphorus intermediate that allows for the introduction of a borane group. Other approaches include stereo-controlled synthesis by means of chiral indole-oxazaphosphorine or chiral oxazaphospholidine. Both of these approaches initially used for the stereocontrolled synthesis of phosphorothioates have been successfully adapted to boranophosphates (Li et al., Chem Rev 107: 4746, 2007). In yet another approach to the production of the stereocontrolled synthesis of oligos linked by boranophosphates involves the use of H-phosphonate intermediates (Iwamato et al., Nucleic Acids Sym Ser 53: 9, 2009). The template directed enzymatic polymerization method and chemical methods have been used by Shaw and her colleagues (Shaw et al., Nucleic Acids Symp Series 52: 655-656, 2008; Hall et al., Nucleic Acids Res 34: 2773-81, 2006).

### 2. Amide Linkage:

The amide linkage (FIG. 16D) can be used in the initial 5′-end linkage site of an agsRNAi between nucleoside positions 1 and 2 and in one or more of the linkages between the units of the overhang precursors and/or in the linkage that joins the overhang precursor to the nucleoside in position 19. An advantage of using this linkage in these positions is that it increases the exonuclease resistance above what is provided by the phosphorothioate linkage. In addition to increasing the stability of the agsRNAi the amide linkage will allow nucleosides to have nucleosides with ribose or with the 2′-fluoro modification to be used in nucleoside position 1 and/or in the 3′end terminal linkage site of an overhang precursor. As discussed in the section dealing with the 5′-end modifications this arrangement can promote the ability of cellular enzymes to add a phosphate group to the 5′-end of the agsRNAi lacking one and thus improve its affinity with an argonaute protein. The use of an amide linkage in these positions of an siRNA guide strand has been previously reported (Iwase et al., Nucleic Acids Symposium Series 53: 119, 2009; Mutisya et al. Nucleic Acids Res 42: 6542-51, 2014).

### 3. N3′ Phosphoramidate Linkage: (FIG. 16C)

This linkage provides greater nuclease resistance than the phosphorothioate linkage and it promotes the C3′-endo conformation in the nucleosides it links. For example, when used to link nucleosides in a DNA oligo it drives the sugars into the C3′endo conformation that DNA strands do not normally have (Egli and Gryaznov, CMLS Cell Mol Life Sci 57: 1440-56, 2000).

When injected into mice antisense oligos with this linkage distribute to various tissues in the animals where they have biologic activity (Gryaznov, Chemisty & Biodiversity 7: 477-93, 2010). This implies that like phosphorothioates N3′ Phosphoramidate linked oligos are sufficiently adherent to plasma proteins to inhibit their clearance from the plasma by the kidneys but not so adherent as to prevent them from being taken up in tissues.

N3′ phosphoramidate linkages can be used in place of phosphorothioate and/or phosphodiester linkages in one or more positions in agsRNAi compounds. They are particularly preferred for use in any purine rich areas that might be found in an agsRNAi and are particularly preferred in seed vehicles used in the modular approach to generate ags-MiRs.

### J. Methods for Administering AgsRNAi Compounds to Subjects

The methods for administering agsRNAi compounds to subjects are essentially the same as for RNAse H1 dependent antisense oligos (Butler et al., Laboratory Investigation 77: 379-88, 1997; Antisense Drug Technology: Principles, Strategies, and Applications, 2^{nd} ed., Stanley T. Crooke (ed.) CRC Press July 2007; Bennett and Swayze, Annu Rev Pharmacol Toxicol 50: 259-93, 2010; Yu et al., Expert Opin Drug Metab Toxicol 9: 169-82, 2013). The ideal length for antisense oligos used as therapeutics is about 20 nucleosides, with about 70% or more of the linkages being phosphorothioate. Consequently, antisense oligo drugs demonstrate a high level of pharmacokinetic behavior that is consistent among oligos with different mechanisms of action, with various sequences, and across several species including those commonly involved in drug development such as rodents, dogs and monkeys. This consistency allows pharmacokinetic data to be modeled and extrapolated between animals and humans.

Antisense oligos can be effectively administered by a number of routes including i.v., s.c., i.m., topical, inhalation, and intrathecal. The effect of the variations in routes of bolus systemic administration on pharmacokinetics involves changes in the rate at which the peak plasma concentration is achieved. The range is minutes to hours. As long as the dose does not overwhelm the ability of the plasma proteins to bind the oligo, the ultimate effect of different routes of systemic administration on ultimate tissue uptake is modest.

When administered i.v., s.c. or i.m. single stranded oligo therapeutics are typically administered multiple times during week one and then weekly, biweekly or monthly to maintain the effect on the target. Subcutaneous administrations are generally preferred for both animals and humans. In mice the oligo doses can be up to about 25 mg/kg s.c. twice daily for multiple days. In the case of humans a single s.c. administration is limited to a volume of about 1 ml and this sets a limit of about 500 mg of oligo due to the viscosity of the oligo solution. As for mice there can be multiple loading doses in humans in the first week followed by week, biweekly or monthly maintenance doses.

In certain embodiments, (e.g., for the treatment of lung disorders, such as pulmonary fibrosis or asthma or to allow for self administration for local or systemic purposes) it may desirable to deliver the oligos described herein in aerosolized form. A pharmaceutical composition comprising at least one oligo can be administered as an aerosol formulation that contains the oligos in dissolved, suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages as drops or sprays. Nasal solutions are generally prepared to be similar to nasal secretions and are generally isotonic and slightly buffered to maintain a pH of about 5.5 to about 6.5, although pH values outside of this range can also be used. Antimicrobial agents or preservatives can also be included in the formulation.

An aerosol formulation for use in inhalations and inhalants is designed so that the oligos are carried into the respiratory tree of the patient. See (WO 01/82868; WO 01/82873; WO 01/82980; WO 02/05730; WO 02/05785 Inhalation solutions can be administered, for example, by a nebulizer Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the oligos. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Remington's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, hydrogen-containing fluorocarbon propellants, and hydrogen-containing chlorofluorocarbon propellants. Halocarbon propellants are described in Johnson, U.S. Pat. No. 5,376,359, and Purewal et al., U.S. Pat. No. 5,776,434.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as numerous other ethers.

The oligos can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, 1990; Purewal et al., U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol consists of a solution of an active ingredient such as oligos in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient peptide and a propellant and can include any combination of solvents and preservatives or antioxidants.

An aerosol formulation can also be a dispersion or suspension. A suspension aerosol formulation will generally contain a suspension of an effective amount of the oligos and a dispersing agent. Dispersing agents useful in the invention include, for example, sorbitan trioleate, oleyl alcohol, oleic acid, lecithin and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient, the oligos. The surfactant can be nonionic, anionic or cationic. One example of an emulsion can include, for example, ethanol, surfactant, water and propellant. Another example of an emulsion can include, for example, vegetable oil, glyceryl monostearate and propane.

Oligos may be formulated for oral delivery (Tillman et al., J Pharm Sci 97: 225, 2008; Raoof et al., J Pharm Sci 93: 1431, 2004; Raoof et al., Eur J Pharm Sci 17: 131, 2002; U.S. Pat. No. 6,747,014; US 2003/0040497; US 2003/0083286; US 2003/0124196; US 2003/0176379; US 2004/0229831; US 2005/0196443; US 2007/0004668; US 2007/0249551; WO 02/092616; WO 03/017940; WO 03/018134; WO 99/60012). Such formulations may incorporate one or more permeability enhancers such as sodium caprate that may be incorporated into an enteric-coated dosage form with the oligo.

There are also delivery mechanisms applicable to oligos with or without carriers that can be applied to particular parts of the body such as the CNS. These include the use of convection-enhanced delivery methods such as but not limited to intracerebral clysis (convection-enhanced microinfusion into the brain—Jeffrey et al., Neurosurgery 46: 683, 2000) to help deliver the cell-permeable carrier/oligo complex to the target cells in the CNS as described in WO 2008/033285.

Drug delivery mechanisms based on the exploitation of so-called leverage-mediated uptake mechanisms are also suitable for the practice of this invention (Schmidt and Theopold, Bioessays 26: 1344, 2004). These mechanisms involve targeting by means of soluble adhesion molecules (SAMs) such as tetrameric lectins, cross-linked membrane-anchored molecules (MARMs) around lipoproteins or bulky hinge molecules leveraging MARMs to cause a local inversion of the cell membrane curvature and formation of an internal endosome, lysosome or phagosome. More specifically leverage-mediated uptake involves lateral clustering of MARMs by SAMs thus generating the configurational energy that can drive the reaction towards internalization of the oligo carrying complex by the cell. These compositions, methods, uses and means of production are provided in WO 2005/074966.

The following materials and methods are provided to facilitate the practice of the present invention. Other approaches for synthesizing and characterizing the agsRNAi of the invention are known to the skilled artisan and are within the scope of the present invention. The methods provided here are not intended to limit the invention in any way.

### Synthesis of 5′-MP and 5′-VP ss-siRNAs

ssRNA are synthesized on ABI 394 synthesizer (1-2 mmol scale) or on GE Healthcare Bioscience A\ddot{}KTA oligopilot synthesizer (40-200 mmol scale) by the phosphoramidite coupling method on an UnyLinker solid support (Guzaev and Manoharan, 2003) packed in the column. A 0.1 M solution of 2′-F, 2′-O-Me, and 2′-O-MOE nucleoside phosphoramidites in anhydrous CH₃CN was used for the synthesis. The 2′-O-MOE-S-methyl uridine-5′-deoxy-5′-methylenephosphonate-3′-phosphoramidite, 2′-O-MOE-5-methyluridine-5′-deoxy-5′-vinylphosphonate-3′-phosphoramidite, 2′-O—[N-(decanoyl)-6-aminohexyl]-5-methyluridine-3′-phosphoramidite, and 2′-O-[N-(hexadecanoyl)-6-aminohexyl]-5-methyluridine-3′-phosphoramidite are dissolved in 40% anhydrousvdichloromethane in anhydrous CH₃CN (0.15 M) and used for the synthesis. For the coupling step, the phosphoramidites are delivered 4- to 6-fold excess over the loading on the solid support, and phosphoramidite condensation is carried out for 10 min. A solution of 6% dichloroacetic acid in toluene is used for removing dimethoxytrityl (DMT) group from the 5′ hydroxyl group of the nucleotide. Extended detritylation condition was used to remove the DMT group from the secondary hydroxyl group of the UnyLinker solid support. 4,5-Dicyanoimidazole (0.7 M) in anhydrous CH₃CN was used as activator during coupling step. Phosphorothioate linkages are introduced using 0.2M solution of phenylacetyl disulfide in 1:1 pyridine/CH3CN as sulfur transfer reagent and treated for 3 min except for the coupling of 5′-deoxy-5′methylenephosphonate and 5′-deoxy-5′-vinylphosphonate phosphoramidites. Phosphorothioate linkages are introduced using a solution of 3-((dimethylaminomethylene)amino)-3H-1,2,4-dithiazole-5-thione (0.05 M, DDTT) in 1:1 pyridine/CH₃CN and a 3 min contact. Solid support-bound ssRNAs are washed with CH₂Cl₂ and dried under high vacuum for 4 hr. The ssRNAs are suspended in a solution of iodotrimethylsilane and pyridine in ichloromethane (dissolve 0.75 ml iodotrimethylsilane and 0.53 ml pyridine in 28.2 ml CH₂Cl₂, use 0.5 ml per mmol of solid support) and allowed to shake at room temperature for 30 min. Reaction is quenched with 50% triethylamine in CH₃CN containing 1 M 2-mercaptoethanol (0.5 ml per mmol of solid support). Supernatant is decanted and the solid support washed with 1:1 triethylamine/CH₃CN containing 1 M 2-mercaptoethanol (2 3 0.5 ml per mmol of solid support). A solution of 1:1 triethylamine/CH₃CN containing 1M 2-mercaptoethanol (0.5 ml per mmol of solid support) is added and kept at room temperature for 45 min. Supernatant is decanted, and the residue aqueous ammonia (28-30 WT %) containing 1M 2-mercaptoethanol (0.75 ml per mmol of solid support) is added and heated at 55.0 for 2 hr. The reaction mixture is allowed to come to room temperature and kept for an additional 24 hr. The solid support is filtered and washed thoroughly with water. The filtrate and the washing are combined together and then cooled in an ice bath and neutralized with glacial acetic acid. The resulting colloidal solution is allowed to stand at −20.0 for 2-3 hr. The precipitate formed is collected by centrifugation followed by decanting the supernatant. The precipitated ssRNAs are then dissolved in water and purified by high pressure liquid chromatography on a reverse phase column (Waters X-Bridge C-18 5 mm, 193 250 mm, A=5 mM tributylammonium acetate in 5% aqueous CH₃CN, B=CH₃CN, 0 to 90% B in 80 min, flow 7 ml min-1,1=260 nm). The fractions were analyzed by LC-MS, and fractions containing full-length ssRNAs pooled together. The tributylammonium counter ions can be exchanged with sodium ions by high-pressure liquid chromatography on a strong anion exchange column (GE Healthcare Bioscience, Source 30Q, 30 mm, 2.54 3 8 cm, A=100 mM ammonium acetate in 30% aqueous CH₃CN, B=1.5 M NaBr in A, 0%-40% of B in 60 min, flow 14 ml min-1,1=260 nm). Desalting by HPLC on a reverse-phase column can give ssRNAs in an isolated yield of 15%-30% based on the initial loading on the solid support. ssRNAs are characterized by ion-pair-HPLC-coupled MS analysis with Agilent 1100 MSD system. Further details on this process are provided in Lima et al. Cell 50:883-894 (2012).

In Vitro Potency of ss-siRNA and siRNA in Primary Hepatocytes

### Liver Perfusion

Mouse liver is perfused as previously described (Graham et al., 1998; Graham et al., 2001; Berry and Friend, 1969) with minor modifications. Briefly, mice are anesthetized with an intraperitoneal injection of tribromoethanol (Avertin). Inferior vena cava is catheterized and clamped. Liver is perfused with Hank's Balanced Salt Solution (life technologies) and mesenteric vessel is cut for drainage. Liver was subsequently perfused with collagenase (Roche). Following the perfusion, liver is removed and gently massaged through sterile nylon mesh. Cells are washed in Williams E (life technologies) containing 10% fetal calf serum, HEPES, L-glutamine and antibiotic/antimycotic. Parenchymal cells are separated from non-parenchymal via centrifugation.

### Transfection

Cells are seeded in 96 well plates at 5,000-10,000 cells/well 16 hr prior to treatment with the exception of liver hepatocytes which are immediately plated and transfected two hours post perfusion. Transfection is performed at indicated concentrations using Opti-MEM medium (life technologies) containing 4-6 μg/ml Lipofectamine 2000 (Life Technologies) for 4 hr at 37° C. Growth medium, DMEM for HeLa and MEF cell lines and Williams E for hepatocytes, is replaced and cells incubated overnight at 37° C. in 5% CO₂. Cells are lysed 16 hr post transfection and total RNA purified using RNeasy 3000 Bio Robot (QIAGEN). Reduction of target mRNA is determined by qRT-PCR as described below. Target mRNA levels can be normalized to total RNA using RiboGreen (Life Technologies). IC₅₀ curves and values are generated using Prism 4 software (GraphPad).

Electroporation of ss-siRNAs

10×ss-siRNA is diluted to 1× by liver hepatocytes at a cell density of 35,000 cells/well. Cells are electroporated at 165 V and a pulse length of 6 ms. Cell/oligo mix is transferred to a 96-well tissue culture plate containing Williams E media and 10% FBS and incubated 16 hr prior to lysis. RNA purification and qRT-PCR are run as described above.

In Vitro Potency of ss-siRNA and siRNA in Transfected HeLa Cells

HeLa cells are seeded in 96 well plates at 5,000-10,000 cells/well 16 hr prior to treatment with the exception of liver hepatocytes which are immediately plated and transfected two hours post perfusion. Transfection is performed at indicated concentrations using Opti-MEM medium (life technologies) containing 4-6 μg/ml Lipofectamine 2000 (Life Technologies) for 4 hr at 37° C. Growth medium, DMEM for HeLa and MEF cell lines and Williams E for hepatocytes, is replaced and cells incubated overnight at 37° C. in 5% CO₂. Cells are lysed 16 hr post transfection and total RNA purified using RNeasy 3000 Bio Robot (QIAGEN). Reduction of target mRNA is determined by qRT-PCR as described herein. Target mRNA levels can be normalized to total RNA using RiboGreen (life technologies). IC50 curves and values are generated using Prism 4 software (GraphPad Prism regression analysis Software).

In Vivo Activity of ss-siRNA in Mice

Animal experiments can be conducted according to American Association for the Accreditation of Laboratory Animal Care guidelines and will be approved by the Animal Welfare Committee (Cold Spring Harbor Laboratory's Institutional Animal Care and Use Committee guidelines). Male Balb/c mice (Charles River Laboratories), aged 6-8 weeks, are maintained at a constant temperature of 23° C. and allowed to standard lab diet and water. Dosing solutions are prepared in phosphate-buffered saline, sterile filtered and quantified. Mice are dosed by single administration (n=4), intravenous or subcutaneous, injection with the exception of subcutaneous doses above 50 mg/kg which consisted of subdivided injections of 25 mg/kg twice a day for indicated number of days. Mice are sacrificed 48 hr post treatment. Animals are anesthetized with isoflurane and terminal bleed performed. Immediately following terminal blood draw, mice are sacrificed by cervical dislocation while under anesthesia. Liver, kidney, and spleen weights are taken and liver tissue homogenized in guanidine isothiocyanate (life technologies) containing 8% β-mercaptoethanol (Sigma) immediately following the sacrifice. Liver homogenate is loaded onto Purelink PCR columns (life technologies) and total RNA purified according to manufacture instructions. Reduction of target mRNA expression is determined by qRT-PCR as previously described (4). Target mRNA levels were normalized to cyclophilin levels and values were confirmed by RiboGreen.

### 5′-RACE

Male Balb/c mice are subcutaneously injected with 25 mg/kg ss-siRNA every two hours for a combined dose of 100 mg/kg. Animals are sacrificed 6 hr post final injection. Liver tissue is homogenized and purified as described above. RNA Ligase Mediated Rapid Amplification of cDNA Ends (5′-RACE) (life technologies) is performed on 1 ug of purified total RNA following manufacture instructions. PCR products are cloned using Topo TA-Machl-Tl cells (life technologies) as directed by manufacture protocol. DNA is purified (QIAGEN) from cultured colonies and sequenced.

Determination of ssRNA Liver Concentrations by Hybridization-Dependent Nuclease ELISA

Liver samples (10-100 mg) are digested with 500 μl proteinase K digestion buffer (5U proteinase K (Sigma, St. Louis, Mo.)/1 ml Buffer G2 (QIAGEN, Hilden, Germany)) for about 1 hr at 40° C. Standard curves are prepared with each analyte at 0.01 μM-5 μM in 500 μl control tissue homogenate (100 mg control liver/ml proteinase K digestion buffer) and digested 1 hr at 40° C. along with study samples. Study samples and standard curves are diluted 1:100 in blank liver digest and 25 μl hybridized with 475 μl 3 nM complementary hybridization probe that included a 5′ digoxigenin and 3′ biotin for 2 hr at room temperature. 200 μl hybridization mix is added to NeutrAvidin-coated 96-well plates (Thermo, Rockford, Ill.) and incubated at room temperature for 1 hr. NeutrAvidin plates are washed with 0.2% Tween 20 in Tris-buffered saline (TBST) and 300 uL 50-300U/ml Si nuclease (Life Technologies, Carlsbad, Calif.) is added and incubated at room temperature for 2 hr. NeutrAvidin plates are washed with TBST and 200 μl 1:2000 anti-Digoxigenin-AP (Roche, Mannheim, Germany) is added and incubated for at least 1 hr at room temperature. NeutrAvidin plates are washed with TBST and 200 μl Attophos (Promega, Madison Wis.) added and fluorescence monitored (excitation 450/50, emission 580/50) using a SpectraMax Gemini microplate reader (Molecular Devices, Sunnyvale, Calif.). Catalysis of Attophos is stopped by addition of 100 μl saturated solution of disodium phosphate (25% Na2HPO4) before final quantitation of fluorescence on microplate reader.

Determination of Tissue Concentrations and Metabolites of ssRNAs Using LC-MS

Tissues are minced and 50-200 mg samples homogenized in 500 μl homogenization buffer (0.5% NP40 substitute (Calbiochem) in Tris-buffered saline, pH8) with homogenization beads (Mo Bio Laboratories, Carlsbad, Calif.) on a Retsch shaker (Mo Bio). Standard curves of each ss-siRNA are established in 500 μl aliquots control tissue homogenate (50-200 mg/ml homogenization buffer). Control oligonucleotide is added as an internal standard (Int. Std.) to all standard curves and study samples. Samples and curves are extracted with phenol/chloroform followed by solid-phase extraction (SPE) of the resulting aqueous extract using phenyl-functionalized silica sorbent (Biotage, Upsalla, Sweden). Eluate from SPE is dried down using a warm forced-air (argon) evaporator and reconstituted in 100-200 μl 4M urea, 25 mM EDTA. Samples are analyzed by LC-MS. In brief, separation is accomplished using an 1100 HPLCMS system (Agilent Technologies, Wilmington, Del.) consisting of a quaternary pump, UV detector, a column oven, an autosampler, and a single quadrupole mass spectrometer. Samples are injected on a X-bridge OST C18 column (2.1 mm×50 mm, 2.5 μm particles; Waters, Milford, Mass.) equipped with a SecurityGuard C18 guard column (Phenomenex, Torrance, Calif.). The columns are maintained at 55° C. Tributylammonium acetate buffer (5 mM) and acetonitrile are used as the mobile phase at a flow rate of 0.3 ml/min. Acetonitrile is increased (gradient) from 20% to 70% over 11 min. Mass measurements are made online using a single quadrupole mass spectrometer scanning 1000-2100 m/z in the negative ionization mode. Molecular masses are determined using ChemStation analysis package (Agilent, Santa Clara, Calif.). Manual evaluation is performed by comparing a table of calculated m/z values corresponding to potential metabolites with the peaks present in a given spectrum. Peak areas from extracted ion chromatograms are determined for ss-siRNAs, 3′ N-1 metabolites, and Int. Std. and a trendline established using the calibration standards, plotting concentration of ssRNA against the ratio of the peak areas ssRNA: Int. Std. Concentration of ssRNAs and 3′ N-1 metabolites in study samples are determined using established trendlines and reported as μg/g tissue.

In Vitro mRNA Knockdown

Mouse Hepa1-6 cells are cultured in Dulbecco's modified Eagle Medium supplemented with 10% fetal bovine serum, 1% penicillin-steptomycin and 1% sodium bicarbonate. These cells are plated in a 96-well culture plates at a density of 3000 cells/well 24 h prior to transfection. Transfections are performed using Opti-MEM I Reduced Serum Media and Lipofectamine RNAiMAX as previously described (30). Final siRNA concentrations range from 100 to 1 nM for in vitro cell-based screens with concentrations varying for ssRNA (100 nM, 10 nM) and dsRNA (100 nM, 10 nM, and 1 nM) (see Tables). Final siRNA concentrations for the dose-response curves can range from 40 to 0.002 nM along an eight-point, 4-fold titration curve. Twenty-four hours post-transfection cells are washed with phosphate-buffered saline and processed using the TaqMan Gene Expression Cells-to-CT (Invitrogen), per manufacturer's instructions, to extract RNA, synthesize cDNA and perform RT-qPCR using an target specific Taqman primer/probe set on an ABI Prism 7900HT Sequence Detector. Reverse transcription conditions can be as follows: 60 min at 37° C. followed by 5 min at 95° C. RT-qPCR conditions were as follows: 2 min at 50° C., 10 min at 95° C., followed by 40 cycles of 15 s at 95° C., and 1 min at 60° C. Gapdh mRNA levels can be used for data normalization (Taqman part number 4308313). Knockdown of targets was calculated as the percent knockdown in target cDNA measured in experimentally treated cells relative to the target cDNA levels measured in non-targeting, control-treated cells. The comparative Ct calculation method for knockdown has previously been described (31). Briefly, ΔCt=Ct_{Target}−Ct_{GADPH} and ΔΔCt=ΔCt(exp)−ΔCt(ctrl) and relative expression level=2^{-ΔΔCt} and % knockdown=100×(1−2^{-ΔΔct}). Potency (EC50) can be calculated using a four-parameter curve fit tool and Prism graphing software (GraphPad Software).

### Lipid Nanoparticle Formulation

siRNA lipid nanoparticles (LNPs) are assembled by simultaneous mixing of a lipid mixture in ethanol with an aqueous solution of siRNA followed by diafiltration. The cationic lipid CLinDMA (2-{4-[(3b)-cholest-5-en-3-yloxy]-butoxy}-N,N-dimethyl-3-[(9Z,12Z)-octadeca-9,12-dien-1-yloxy]propan-1-amine), cholesterol and PEG-DMG (monomethoxy(polyethyleneglycol)-1,2-dimyristoylglycerol) are mixed together at a molar ratio of 50:44:6. PEG-DMG was purchased from NOF Corporation, cholesterol from Northern Lipids, and CLinDMA is synthesized by Merck & Co., Inc. Particle size is measured by dynamic light scattering using a Wyatt DynaPro plate reader and percent encapsulation is determined using a SYBR Gold fluorescence assay (Invitrogen) and are within pre-established quality metrics.

qPCR Measurement

HCT-116 cells, cultured in McCoy's 5A Medium, supplemented with 10% fetal bovine serum and 1% penicillin-streptomycin, and plated at a density of 6000 cells/well in 96-well culture plates 24 h prior to transfection, are transfected with Lipofectamine RNAiMax (Invitrogen) and Opti-MEM I Reduced Serum Media (GIBCO). Twenty-four hours post-transfection, cells are rinsed with phosphate-buffered saline and processed with the Cells-to-CT Kit (Applied Biosystems). TaqMan gene-specific probes are used on an ABI Prism 9700HT Sequence Detector for RT-qPCR. Reverse transcription conditions are as follows: 2 min at 50° C., 10 min at 95° C., followed by 40 cycles of 15 sec at 95° C., and 1 min at 60° C. GUSB mRNA levels can be used for data normalization. Knockdown is calculated as described above.

### Microarray and Bioinformatics Analysis

HCT-116 cells are transfected with 10 nM miRNA using conventional techniques. RNA is extracted using RNeasy (Qiagen), amplified using the Ovation protocol (Nugen), and profiled on custom Affymetrix arrays (Rosetta Custom Human 2.0, Affymetrix). Array signals are analyzed with Affymetrix GeneChip Operating Software and Affymetrix Power Tools. UTR hexamer and heptamer enrichment is analyzed using the hypergeometric distribution.

Oligonucleotide sequences are shown in Tables 6-21.

### Nucleoside and Linkage Modification Key for Examples of Agsrnai Compounds Found in Tables 6-21

Each of the possible strand modifications in the following tables is designated according to the following abbreviations. Subscripts follow the standard canonical base indicated by a letter A, T, U, C or G where a subscript indicates the base substitute, if any, and another subscript the sugar, sugar analog or sugar substitute being used. When the sugar used in a nucleoside is not specifically designated by a subscript it is ribose. Letters indicate sugar modifications or sugar substitutes while a number indicates base substitutions. The standard bases that can be substituted for by a particular non-canonical base appear in parenthesis after the name of the modified base in the key below. Other abbreviations, as indicated below, are applied to 5′-end terminal modifications, linkages or overhang precursors.

Nucleoside Sugars:
1) 2′-deoxyribose is D
2) 2′-fluoro is F
3) 2′-O-methyl is M
4) 2′-O-methyoxyethyl is moe
5) AENA is B
6) ALN is H
7) ANA is J
8) CENA is K
9) CRN is W for R monomer and V for Q monomer based
10) EA is E
11) FANA is Z
12) HM is Q
13) HNA is S
14) FHNA is Y
15) LNA is L
16) UNA is N
17) CeNA is O
18) F-CeNA is T
19) cEt is cet
20) Thio-LNA is TL
21) Amino-LNA is I

Nucleoside Bases:
1) Abasic (A, C, G or U) is 0
2) 2,6-Diaminopurine (A) is 2
3) 2-thiouracil (U) is 3
4) 4-thiouracil (C) is 4
5) 5-nitroindole (A, C, G or U) is 5
6) 2,4-difluorotoluene (A, C, G or U) is 6
7) 2-thiothymine (U) is 7
8) N⁴,N⁴-dimethylcytosine (A, C, G or U) is 8
9) Purine (A, C, G or U) is 9
10) 2-aminopurine (A, C, G or U) is 10
11) 5,6-dihydrouracil (A, C, G or U) is 11
12) Pseudouracil (U) is 12
13) 2,4-dichlorobenzene (A, C, G or U) is 13
14) 3-methyluracil (A, C, G or U) is 14
15) N²-cyclopentylguanine (G) is 15
16) N²-propyl-2-aminopurine (A) is 16
17) N²-cyclopentyl-2-aminopurine (A) is 17
18) 5-methyluracil (U) is 18
19) 5-methylcytosine (C) is 19

### 5′-End Terminal Modifications:

1) When the 5′-end 5′ carbon of the antisense strand is manufactured with a phosphate group a P-precedes the terminal nucleoside designation.
2) 5′-VP specifically designates the 5′-(E)-vinylphosphonate bioisostere of phosphate that contains a double bond between the 5′ and 6′ carbons in a trans configuration as illustrated in FIG. 15 where it is conjugated to a nucleoside. 5′-VP can be conjugated to any terminal 5′-end agsRNAi nucleoside sugar or sugar analog that is provided herein.

### Linkages:

1) Phosphodiester is no linkage indicated
2) Phosphorothioate linkage is indicated by

### 3′-End Overhang Precursors:

Capital X represents some number of overhang precursor units where the composition of the units and their number are chosen from those provided herein. Overhang precursors can have any of the units and linkages provided herein for this use.

### Table 6

### Examples of Murine/Human mIR-34a-5p ags-MIR Compositions

A. Murine/Human miR-34a-5p Sequence:

The miRBase accession numbers for murine and human miR-34a-5p are MI0000584 and MI0000268. The guide strand from each species has the same sequence. The sequence minus the overhang is as follows:

UGGCAGUGUCUUAGCUGGUUGU

B. Some Examples of Ags-MiR Mimics of Murine/Human miR-34a:

As for any ssRNAi the endogenous miR-34a sequence minus the overhang can be modified for basic nuclease resistance and then further modified by AGSD. The resulting compound can have any of the overhang precursors provided herein.

Since the targeting code for miR-34a is a seed sequence ags-MiR mimics of miR-34a can be generated using the modular approach. A 9-mer 5′-end module sequence that includes the seed sequence of human and murine miR-34a-5p suitable for use as a modular component is the following:

### UGGCAGUGU

The 5′-end terminal nucleoside of a guide strand can have any base suitable for use in the present invention. In this design example the 5′-end terminal U found in the endogenous miRNA guide strand is retained.

This 9-mer 5′-end module can be used with any 10-mer seed vehicle sequence such as those shown in Table 19A. An example of such a combination is provided in the Modular Design Example Set #1. Alternatively, an 8-mer 5′-end module can be used with an 11-mer seed vehicle sequence such as those found in Table 3. An example of this combination is provided in Modular Design Example Set #2.

### 1) Modular Design Example Set #1:

In this example the seed vehicle sequence is vehicle 9y_2 found in Table 19A. It is as follows:

CGCUUCUCUU

Any of the 5′-end nucleosides (P-U_{M} or P-A_{M}) shown in the examples of agsRNAi compounds in Tables 6A and 6B can be replaced with any of those provided for herein including one of the 5′end nucleosides selected from the group consisting of P-U_{F}, P-U_{M}, P-U_{N}, P-U_{L}, P-U_{Z}, P-U_{H}, P-U_{B}, P-U_{K}, P-U_{Q}, P-U_{S}, P-U_{T}, P-U_{W}, P-U_{Y}, P-U_{O}, P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-A_{F}, P-A_{M}, P-A_{L}, P-A_{Z}, P-A_{H}, P-A_{B}, P-A_{K}, P-A_{Q}, P-A_{S}, P-A_{T}, P-A_{W}, P-A_{Y}, P-A_{O}, P-T_{L}, P-T_{H}, 5′-VP-Tₘₒₑ, 5′-VP-T_{E}, 5′-VP-U_{E}, 5′-VP-U_{Z}, 5′-VP-U_{K}, 5′-VP-U_{Q}, 5′-VP-U_{B}, 5′-VP-T_{Z}, 5′-VP-T_{K}, 5′-VP-T_{Q} and 5′-VP-T_{B}.

Any of the nucleosides in Table 6A or 6B with an L modification(s) found in one or more nucleosides in positions 2-19 can have any given L or set of Ls replaced with any of those sugar or sugar analogs provided for herein for positions 2-19 including one or more of the sugar, sugar analogs or sugar substitutes independently selected from the group consisting of: S, J, W, V, Q, Y, O, T and cet (i.e., if multiple Ls are replaced the selected replacements do not all have to be the same sugar or sugar analog).

Any of the nucleosides in Tables 6A or 6B with an L modification found in the overhang precursor can have the L replaced with any of those units and linkages provided for herein for overhang precursors including one or more of the sugar, sugar analogs or sugar substitutes independently selected from the group consisting of F, J, W, V, Y, T, TL and I.

Any of the compounds in Table 6A or 6B can have any of the overhang precursors provided for herein.

TABLE 6A
Compositions of Matter Suitable for Administration to a Subject
with or without a Protective Carrier and for Any Other use as
Provided for Herein (5′-3′)

P-U_{M}~G_{F}~G_{M}C_{F}~A_{M}G_{F}~U_{M3}G_{F}~U_{M3}C_{F}~G_{M}C_{F}~U_{M3}U_{F}~C_{M}U_{F3}~C_{M}~U_{F3}~U_{M}~A_{F}~G_{F}~A_{M}
P-U_{M}~G_{F}~G_{M}C_{F}~A_{M}G_{F}~U_{M}G_{F}~U_{M3}C_{F}~G_{M}C_{F}~U_{M3}U_{F}~C_{M}U_{F3}~C_{M}~U₃~U_{M}~A_{F}~G_{F}~A_{M}
P-U_{M}~G_{F}~G_{M}C_{F}~A_{M}G_{F}~U_{M3}G_{F}~U_{M}C_{F}~G_{M}C_{F}~U_{M3}U_{F}~C_{M}U_{F}~C_{M}~U_{F}~U_{M3}~A_{F}~G_{F}~A_{M}
P-U_{M}~G_{F}~G_{M}C_{F}~A_{M}G_{F}~U_{M3}G_{F}~U_{M3}C_{F}~G_{M}C_{F}~U_{M3}U_{F3}~C_{M}U_{F3}~C_{M}~U_{F3}~U_{M3}~A_{F}~G_{F}~A_{M}
P-U_{M}~G_{F}~G_{M}C_{F4}~A_{M}G_{F}~U_{M3}G_{F}~U_{M3}C_{F}~G_{M}C_{F}~U_{M3}U_{F}~C_{M}U_{F3}~C_{M4}~U_{F3}~U_{M}~A_{F}~G_{F}~A_{M}
P-U_{M}~G_{F}~G_{M}C_{F4}~A_{M}G_{F}~U_{M}G_{F}~U_{M3}C_{F}~G_{M}C_{F4}~U_{M3}U_{F}~C_{M4}U_{F3}~C_{M}~U_{F3}~U_{M}~A_{F}~G_{F}~A_{M}
P-U_{M}~G_{F}~G_{M}C_{F4}~A_{M}G_{F}~U_{M3}G_{F}~U_{M}C_{F4}~G_{M}C_{F4}~U_{M3}U_{F}~C_{M4}U_{F}~C_{M4}~U_{F}~U_{M3}~A_{F}~G_{F}~A_{M}
P-U_{M}~G_{F}~G_{M}C_{F4}~A_{M}G_{F}~U_{M3}G_{F}~U_{M3}C_{F4}~G_{M}C_{F4}~U_{M3}U_{F3}~C_{M4}U_{F3}~C_{M4}~U_{F3}~U_{M3}~A_{F}~G_{F}~A_{M}
P-U_{M}~G_{F}~G_{M}C_{F}~A_{M}G_{F}~U_{M12}G_{F}~U_{M12}C_{F}~G_{M}C_{F}~U_{M12}U_{F}~C_{M}U_{F12}~C_{M}~U_{F12}~U_{M}~A_{F}~G_{F}~A_{M}
P-U_{M}~G_{F}~G_{M}C_{F}~A_{M}G_{F}~U_{M}G_{F}~U_{M12}C_{F}~G_{M}C_{F}~U_{M12}U_{F}~C_{M}U_{F12}~C_{M}~U_{F12}~U_{M}~A_{F}~G_{F}~A_{M}
P-U_{M}~G_{F}~G_{M}C_{F}~A_{M}G_{F}~U_{M12}G_{F}~U_{M}C_{F}~G_{M}C_{F}~U_{M12}U_{F}~C_{M}U_{F}~C_{M}~U_{F}~U_{M12}~A_{F}~G_{F}~A_{M}
P-U_{M}~G_{F}~G_{M}C_{F}~A_{M}G_{F}~U_{M12}G_{F}~U_{M12}C_{F}~G_{M}C_{F}~U_{M12}U_{F12}~C_{M}U_{F12}~C_{M}~U_{F12}~U_{M12}~A_{F}~G_{F}~A_{M}
P-U_{M}~G_{F}~G_{M}C_{F4}~A_{M}G_{F}~U_{M12}G_{F}~U_{M12}C_{F}~G_{M}C_{F}~U_{M12}U_{F}~C_{M}U_{F12}~C_{M4}~U_{F12}~U_{M}~A_{F}~G_{F}~A_{M}
P-U_{M}~G_{F}~G_{M}C_{F4}~A_{M}G_{F}~U_{M}G_{F}~U_{M12}C_{F}~G_{M}C_{F4}~U_{M12}U_{F}~C_{M4}U_{F12}~C_{M}~U_{F12}~U_{M}~A_{F}~G_{F}~A_{M}
P-U_{M}~G_{F}~G_{M}C_{F4}~A_{M}G_{F}~U_{M12}G_{F}~U_{M}C_{F4}~G_{M}C_{F4}~U_{M12}U_{F}~C_{M4}U_{F}~C_{M4}~U_{F}~U_{M12}~A_{F}~G_{F}~A_{M}
P-U_{M}~G_{F}~G_{M}C_{F4}~A_{M}G_{F}~U_{M12}G_{F}~U_{M12}C_{F4}~G_{M}C_{F4}~U_{M12}U_{F12}~C_{M4}U_{F12}~C_{M4}~U_{F12}~U_{M12}~A_{F}~G_{F}~A_{M}
P-U_{M}~G_{F}~G_{M}C_{F}~A_{M}G_{F}~U_{M18}G_{F}~U_{M18}C_{F}~G_{M}C_{F}~U_{M18}U_{F}~C_{M}U_{F18}~C_{M}~U_{F18}~U_{M}~A_{F}~G_{F}~A_{M}
P-U_{M}~G_{F}~G_{M}C_{F}~A_{M}G_{F}~U_{M}G_{F}~U_{M18}C_{F}~G_{M}C_{F}~U_{M18}U_{F}~C_{M}U_{F18}~C_{M}~U_{F18}~U_{M}~A_{F}~G_{F}~A_{M}
P-U_{M}~G_{F}~G_{M}C_{F}~A_{M}G_{F}~U_{M18}G_{F}~U_{M}C_{F}~G_{M}C_{F}~U_{M18}U_{F}~C_{M}U_{F}~C_{M}~U_{F}~U_{M18}~A_{F}~G_{F}~A_{M}
P-U_{M}~G_{F}~G_{M}C_{F}~A_{M}G_{F}~U_{M18}G_{F}~U_{M18}C_{F}~G_{M}C_{F}~U_{M18}U_{F18}~C_{M}U_{F18}~C_{M}~U_{F18}~U_{M18}~A_{F}~G_{F}~A_{M}
P-U_{M}~G_{F}~G_{M}C_{F4}~A_{M}G_{F}~U_{M18}G_{F}~U_{M18}C_{F}~G_{M}C_{F}~U_{M18}U_{F}~C_{M}U_{F18}~C_{M4}~U_{F18}~U_{M}~A_{F}~G_{F}~A_{M}
P-U_{M}~G_{F}~G_{M}C_{F4}~A_{M}G_{F}~U_{M}G_{F}~U_{M18}C_{F}~G_{M}C_{F4}~U_{M18}U_{F}~C_{M4}U_{F18}~C_{M}~U_{F18}~U_{M}~A_{F}~G_{F}~A_{M}
P-U_{M}~G_{F}~G_{M}C_{F4}~A_{M}G_{F}~U_{M18}G_{F}~U_{M}C_{F4}~G_{M}C_{F4}~U_{M18}U_{F}~C_{M4}U_{F}~C_{M4}~U_{F}~U_{M18}~A_{F}~G_{F}~A_{M}
P-U_{M}~G_{F}~G_{M}C_{F4}~A_{M}G_{F}~U_{M18}G_{F}~U_{M18}C_{F4}~G_{M}C_{F4}~U_{M18}U_{F18}~C_{M4}U_{F18}~C_{M4}~U_{F18}~U_{M18}~A_{F}~G_{F}~A_{M}
P-U_{M}~G_{F}~G_{M}C_{F}~A_{M}G_{F}~U_{M3}G_{F}~U_{M3}C_{F}~G_{M}C_{F}~U_{M3}U~C_{M}U_{F3}~C_{M}~U_{F3}~U_{M}~A_{F}~G_{F}~A_{M}
P-U_{M}~G_{F}~G_{M}C_{F}~A_{M}G_{F}~U_{M}G_{F}~U_{M3}C_{F}~G_{M}C_{F}~U_{M3}U~C_{M}U_{F3}~C_{M}~U_{F3}~U_{M}~A_{F}~G_{F}~A_{M}
P-U_{M}~G_{F}~G_{M}C_{F}~A_{M}G_{F}~U_{M3}G_{F}~U_{M}C_{F}~G_{M}C_{F}~U_{M3}U~C_{M}U_{F}~C_{M}~U_{F}~U_{M3}~A_{F}~G_{F}~A_{M}
P-U_{M}~G_{F}~G_{M}C_{F}~A_{M}G_{F}~U_{M3}G_{F}~U_{M3}C_{F}~G_{M}C_{F}~U_{M3}U~C_{M}U_{F3}~C_{M}~U_{F3}~U_{M3}~A_{F}~G_{F}~A_{M}
P-U_{M}~G_{F}~G_{M}C_{F4}~A_{M}G_{F}~U_{M3}G_{F}~U_{M3}C_{F}~G_{M}C_{F}~U_{M3}U~C_{M}U_{F3}~C_{M4}~U_{F3}~U_{M}~A_{F}~G_{F}~A_{M}
P-U_{M}~G_{F}~G_{M}C_{F4}~A_{M}G_{F}~U_{M}G_{F}~U_{M3}C_{F}~G_{M}C_{F4}~U_{M3}U~C_{M4}U_{F3}~C_{M}~U_{F3}~U_{M}~A_{F}~G_{F}~A_{M}
P-U_{M}~G_{F}~G_{M}C_{F4}~A_{M}G_{F}~U_{M3}G_{F}~U_{M}C_{F4}~G_{M}C_{F4}~U_{M3}U~C_{M4}U_{F}~C_{M4}~U_{F}~U_{M3}~A_{F}~G_{F}~A_{M}
P-U_{M}~G_{F}~G_{M}C_{F4}~A_{M}G_{F}~U_{M3}G_{F}~U_{M3}C_{F4}~G_{M}C_{F4}~U_{M3}U~C_{M4}U_{F3}~C_{M4}~U_{F3}~U_{M3}~A_{F}~G_{F}~A_{M}
P-U_{M}~G_{F}~G_{M}C_{F}~A_{M}G_{F}~U_{M12}G_{F}~U_{M12}C_{F}~G_{M}C_{F}~U_{M12}U~C_{M}U_{F12}~C_{M}~U_{F12}~U_{M}~A_{F}~G_{F}~A_{M}
P-U_{M}~G_{F}~G_{M}C_{F}~A_{M}G_{F}~U_{M}~G_{F}~U_{M12}C_{F}~G_{M}C_{F}~U_{M12}U~C_{M}U_{F12}~C_{M}~U_{F12}~U_{M}~A_{F}~G_{F}~A_{M}
P-U_{M}~G_{F}~G_{M}C_{F}~A_{M}G_{F}~U_{M12}G_{F}~U_{M}C_{F}~G_{M}C_{F}~U_{M12}U~C_{M}U_{F}~C_{M}~U_{F}~U_{M12}~A_{F}~G_{F}~A_{M}
P-U_{M}~G_{F}~G_{M}C_{F}~A_{M}G_{F}~U_{M12}G_{F}~U_{M12}C_{F}~G_{M}C_{F}~U_{M12}U~C_{M}U_{F12}~C_{M}~U_{F12}~U_{M12}~A_{F}~G_{F}~A_{M}
P-U_{M}~G_{F}~G_{M}C_{F4}~A_{M}G_{F}~U_{M12}G_{F}~U_{M12}C_{F}~G_{M}C_{F}~U_{M12}U~C_{M}U_{F12}~C_{M4}~U_{F12}~U_{M}~A_{F}~G_{F}~A_{M}
P-U_{M}~G_{F}~G_{M}C_{F4}~A_{M}G_{F}~U_{M}G_{F}~U_{M12}C_{F}~G_{M}C_{F4}~U_{M12}U~C_{M4}U_{F12}~C_{M}~U_{F12}~U_{M}~A_{F}~G_{F}~A_{M}
P-U_{M}~G_{F}~G_{M}C_{F4}~A_{M}G_{F}~U_{M12}G_{F}~U_{M}C_{F4}~G_{M}C_{F4}~U_{M12}U~C_{M4}U_{F}~C_{M4}~U_{F}~U_{M12}~A_{F}~G_{F}~A_{M}
P-U_{M}~G_{F}~G_{M}C_{F4}~A_{M}G_{F}~U_{M12}G_{F}~U_{M12}C_{F4}~G_{M}C_{F4}~U_{M12}U~C_{M4}U_{F12}~C_{M4}~U_{F12}~U_{M12}~A_{F}~G_{F}~A_{M}
P-U_{M}~G_{F}~G_{M}C_{F}~A_{M}G_{F}~U_{M18}G_{F}~U_{M18}C_{F}~G_{M}C_{F}~U_{M18}U~C_{M}U_{F18}~C_{M}~U_{F18}~U_{M}~A_{F}~G_{F}~A_{M}
P-U_{M}~G_{F}~G_{M}C_{F}~A_{M}G_{F}~U_{M}G_{F}~U_{M18}C_{F}~G_{M}C_{F}~U_{M18}U~C_{M}U_{F18}~C_{M}~U_{F18}~U_{M}~A_{F}~G_{F}~A_{M}
P-U_{M}~G_{F}~G_{M}C_{F}~A_{M}G_{F}~U_{M18}G_{F}~U_{M}C_{F}~G_{M}C_{F}~U_{M18}U~C_{M}U_{F}~C_{M}~U_{F}~U_{M18}~A_{F}~G_{F}~A_{M}
P-U_{M}~G_{F}~G_{M}C_{F}~A_{M}G_{F}~U_{M18}G_{F}~U_{M18}C_{F}~G_{M}C_{F}~U_{M18}U~C_{M}U_{F18}~C_{M}~U_{F18}~U_{M18}~A_{F}~G_{F}~A_{M}
P-U_{M}~G_{F}~G_{M}C_{F4}~A_{M}G_{F}~U_{M18}G_{F}~U_{M18}C_{F}~G_{M}C_{F}~U_{M18}U~C_{M}U_{F18}~C_{M4}~U_{F18}~U_{M}~A_{F}~G_{F}~A_{M}
P-U_{M}~G_{F}~G_{M}C_{F4}~A_{M}G_{F}~U_{M}G_{F}~U_{M18}C_{F}~G_{M}C_{F4}~U_{M18}U~C_{M4}U_{F18}~C_{M}~U_{F18}~U_{M}~A_{F}~G_{F}~A_{M}
P-U_{M}~G_{F}~G_{M}C_{F4}~A_{M}G_{F}~U_{M18}G_{F}~U_{M}C_{F4}~G_{M}C_{F4}~U_{M18}U~C_{M4}U_{F}~C_{M4}~U_{F}~U_{M18}~A_{F}~G_{F}~A_{M}
P-U_{M}~G_{F}~G_{M}C_{F4}~A_{M}G_{F}~U_{M18}G_{F}~U_{M18}C_{F4}~G_{M}C_{F4}~U_{M18}U~C_{M4}U_{F18}~C_{M4}~U_{F18}~U_{M18}~A_{F}~G_{F}~A_{M}
P-U_{M}~G_{F}~G_{L}C_{F}~A_{M}G_{L}~U_{M3}G_{F}~U_{M3}C_{F}~G_{L}C_{F}~U_{M3}U_{F}~C_{M}U_{F3}~C_{L}~U_{F3}~U_{M}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U~C_{M}U_{F3}~C_{L}~U_{F3}~U_{M}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U_{F}~C_{M}U_{F3}~C_{L}~U_{F3}~U_{M}~A_{L}~G_{L}~A_{M}
P-U_{M}~G_{F}~G_{L}C_{F}~A_{M}G_{L}~U_{M}G_{F}~U_{M3}C_{F}~G_{L}C_{F}~U_{M3}U_{F}~C_{M}U_{F3}~C_{L}~U_{F3}~U_{M}~A_{L}~G_{L}~A_{M}
P-U_{M}~G_{F}~G_{L}C_{F}~A_{M}G_{L}~U_{M3}G_{F}~U_{M}C_{F}~G_{L}C_{F}~U_{M3}U_{F}~C_{M}U_{F}~C_{L}~U_{F}~U_{M3}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U_{F3}~C_{M}U_{F3}~C_{L}~U_{F3}~U_{M3}~A_{L}~G_{L}~A_{M}
P-U_{M}~G_{F}~G_{L}C_{F4}~A_{M}G_{L}~U_{M3}G_{F}~U_{M3}C_{F}~G_{L}C_{F}~U_{M3}U_{F}~C_{M}U_{F3}~C_{L4}~U_{F3}~U_{M}~A_{L}~G_{L}~A_{M}
P-U_{M}~G_{F}~G_{L}C_{F4}~A_{M}G_{L}~U_{M}G_{F}~U_{M3}C_{F}~G_{L}C_{F4}~G_{M3}U_{F}~C_{M4}U_{F3}~C_{L}~U_{F3}~U_{M}~A_{L}~G_{L}~A_{M}
P-U_{M}~G_{F}~G_{L}C_{F4}~A_{M}G_{L}~U_{M3}G_{F}~U_{M}C_{F4}~G_{L}C_{F4}~U_{M3}U_{F}~C_{M4}U_{F}~C_{L4}~U_{F}~U_{M3}~A_{L}~G_{L}~A_{M}
P-U_{M}~G_{F}~G_{L}C_{F4}~A_{M}G_{L}~U_{M3}G_{F}~U_{M3}C_{F4}~G_{L}C_{F4}~U_{M3}U_{F3}~C_{M4}U_{F3}~C_{L4}~U_{F3}~U_{M3}~A_{L}~G_{L}~A_{M}
P-U_{M}~G_{F}~G_{L}C_{F}~A_{M}G_{L}~U_{M12}G_{F}~U_{M12}C_{F}~G_{L}C_{F}~U_{M12}U_{F}~C_{M}U_{F12}~C_{L}~U_{F12}~U_{M}~A_{L}~G_{L}~A_{M}
P-U_{M}~G_{F}~G_{L}C_{F}~A_{M}G_{L}~U_{M}G_{F}~U_{M12}C_{F}~G_{L}C_{F}~U_{M12}U_{F}~C_{M}U_{F12}~C_{L}~U_{F12}~U_{M}~A_{L}~G_{L}~A_{M}
P-U_{M}~G_{F}~G_{L}C_{F}~A_{M}G_{L}~U_{M12}G_{F}~U_{M}C_{F}~G_{L}C_{F}~U_{M12}U_{F}~C_{M}U_{F}~C_{L}~U_{F}~U_{M12}~A_{L}~G_{L}~A_{M}
P-U_{M}~G_{F}~G_{L}C_{F}~A_{M}G_{L}~U_{M12}G_{L}~U_{M12}C_{F}~G_{L}C_{F}~U_{M12}U_{F12}~C_{M}U_{F12}~C_{L}~U_{F12}~U_{M12}~A_{L}~G_{L}~A_{M}
P-U_{M}~G_{F}~G_{L}C_{F4}~A_{M}G_{L}~U_{M12}G_{F}~U_{M12}C_{F}~G_{L}C_{F}~U_{M12}U_{F}~C_{M}U_{F12}~C_{L4}~U_{F12}~U_{M}~A_{L}~G_{L}~A_{M}
P-U_{M}~G_{F}~G_{L}C_{F4}~A_{M}G_{L}~U_{M12}G_{F}~U_{M12}C_{F}~G_{L}C_{F4}~U_{M12}U_{F}~C_{M4}U_{F12}~C_{L}~U_{F12}~U_{M}~A_{L}~G_{L}~A_{M}
P-U_{M}~G_{F}~G_{L}C_{F4}~A_{M}G_{L}~U_{M12}G_{F}~U_{M}C_{F4}~G_{L}C_{F4}~U_{M12}U_{F}~C_{M4}U_{F}~C_{L4}~U_{F}~U_{M12}~A_{L}~G_{L}~A_{M}
P-U_{M}~G_{F}~G_{L}C_{F4}~A_{M}G_{L}~U_{M12}G_{F}~U_{M12}C_{F4}~G_{L}C_{F4}~U_{M12}U_{F12}~C_{M4}U_{F12}~C_{L4}~U_{F12}~U_{M12}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U_{F}~C_{M}U_{F18}~C_{L}~U_{F18}~U_{M}~A_{L}~G_{L}~A_{M}
P-U_{M}~G_{F}~G_{L}C_{F}~A_{M}G_{L}~U_{M}G_{F}~U_{M18}C_{F}~G_{L}C_{F}~U_{M18}U_{F}~C_{M}U_{F18}~C_{L}~U_{F18}~U_{M}~A_{L}~G_{L}~A_{M}
P-U_{M}~G_{F}~G_{L}C_{F}~A_{M}G_{L}~U_{M18}G_{F}~U_{M}C_{F}~G_{L}C_{F}~U_{M18}U_{F}~C_{M}U_{F}~C_{L}~U_{F}~U_{M18}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U_{F18}~C_{M}U_{F18}~C_{L}~U_{F18}~U_{M18}~A_{L}~G_{L}~A_{M}
P-U_{M}~G_{F}~G_{L}C_{F4}~A_{M}G_{L}~U_{M18}G_{F}~U_{M18}C_{F}~G_{L}C_{F}~U_{M18}U_{F}~C_{M}U_{F18}~C_{L4}~U_{F18}~U_{M}~A_{L}~G_{L}~A_{M}
P-U_{M}~G_{F}~G_{L}C_{F4}~A_{M}G_{L}~U_{M}G_{F}~U_{M18}C_{F}~G_{L}C_{F4}~U_{M18}U_{F}~C_{M4}U_{F18}~C_{L}~U_{F18}~U_{M}~A_{L}~G_{L}~A_{M}
P-U_{M}~G_{F}~G_{L}C_{F4}~A_{M}G_{L}~U_{M18}G_{F}~U_{M}C_{F4}~G_{L}C_{F4}~U_{M18}U_{F}~C_{M4}U_{F}~C_{L4}~U_{F}~U_{M18}~A_{L}~G_{L}~A_{M}
P-U_{M}~G_{F}~G_{L}C_{F4}~A_{M}G_{L}~U_{M18}G_{F}~U_{M18}C_{F4}~G_{L}C_{F4}~U_{M18}U_{F18}~C_{M4}U_{F18}~C_{L4}~U_{F18}~U_{M18}~A_{L}~G_{L}~A_{M}
P-U_{M}~G_{F}~G_{L}C_{F}~A_{M}G_{L}~U_{M3}G_{L}~U_{M3}C_{F}~G_{L}C_{F}~U_{M3}U~C_{M}U_{F3}~C_{L}~U_{F3}~U_{M}~A_{L}~G_{L}~A_{M}
P-U_{M}~G_{F}~G_{L}C_{F}~A_{M}G_{L}~U_{M}G_{F}~U_{M3}C_{F}~G_{L}C_{F}~U_{M3}U~C_{M}U_{F3}~C_{L}~U_{F3}~U_{M}~A_{L}~G_{L}~A_{M}
P-U_{M}~G_{F}~G_{L}C_{F}~A_{M}G_{L}~U_{M3}G_{F}~U_{M}C_{F}~G_{L}C_{F}~U_{M3}U~C_{M}U_{F}~C_{L}~U_{F}~U_{M3}~A_{L}~G_{L}~A_{M}
P-U_{M}~G_{F}~G_{L}C_{F}~A_{M}G_{L}~U_{M3}G_{L}~U_{M3}C_{F}~G_{L}C_{F}~U_{M3}U~C_{M}U_{F3}~C_{L}~U_{F3}~U_{M3}~A_{L}~G_{L}~A_{M}
P-U_{M}~G_{F}~G_{L}C_{F4}~A_{M}G_{L}~U_{M3}G_{L}~U_{M3}C_{F}~G_{L}C_{F}~U_{M3}U~C_{M}U_{F3}~C_{L4}~U_{F3}~U_{M}~A_{L}~G_{L}~A_{M}
P-U_{M}~G_{F}~G_{L}C_{F4}~A_{M}G_{L}~U_{M}G_{F}~U_{M3}C_{F}~G_{L}C_{F4}~U_{M3}U~C_{M4}U_{F3}~C_{L}~U_{F3}~U_{M}~A_{L}~G_{L}~A_{M}
P-U_{M}~G_{F}~G_{L}C_{F4}~A_{M}G_{L}~U_{M3}G_{F}~U_{M}C_{F4}~G_{L}C_{F4}~U_{M3}U~C_{M4}U_{F}~C_{L4}~U_{F}~U_{M3}~A_{L}~G_{L}~A_{M}
P-U_{M}~G_{F}~G_{L}C_{F4}~A_{M}G_{L}~U_{M3}G_{F}~U_{M3}C_{F4}~G_{L}C_{F4}~U_{M3}U~C_{M4}U_{F3}~C_{L4}~U_{F3}~U_{M3}~A_{L}~G_{L}~A_{M}
P-U_{M}~G_{F}~G_{L}C_{F}~A_{M}G_{L}~U_{M12}G_{F}~U_{M12}C_{F}~G_{L}C_{F}~U_{M12}U~C_{M}U_{F12}~C_{L}~U_{F12}~U_{M}~A_{L}~G_{L}~A_{M}
P-U_{M}~G_{F}~G_{L}C_{F}~A_{M}G_{L}~U_{M}G_{F}~U_{M12}C_{F}~G_{L}C_{F}~U_{M12}U~C_{M}U_{F12}~C_{L}~U_{F12}~U_{M}~A_{L}~G_{L}~A_{M}
P-U_{M}~G_{F}~G_{L}C_{F}~A_{M}G_{L}~U_{M12}G_{F}~U_{M}C_{F}~G_{L}C_{F}~U_{M12}U~C_{M}U_{F}~C_{L}~U_{F}~U_{M12}~A_{L}~G_{L}~A_{M}
P-U_{M}~G_{F}~G_{L}C_{F}~A_{M}G_{L}~U_{M12}G_{F}~U_{M12}C_{F}~G_{L}C_{F}~U_{M12}U~C_{M}U_{F12}~C_{L}~U_{F12}~U_{M12}~A_{L}~G_{L}~A_{M}
P-U_{M}~G_{F}~G_{L}C_{F4}~A_{M}G_{L}~U_{M12}G_{L}~U_{M12}C_{F}~G_{L}C_{F}~U_{M12}U~C_{M}U_{F12}~C_{L4}~U_{F12}~U_{M}~A_{L}~G_{L}~A_{M}
P-U_{M}~G_{F}~G_{L}C_{F4}~A_{M}G_{L}~U_{M}G_{F}~U_{M12}C_{F}~G_{L}C_{F4}~U_{M12}U~C_{M4}U_{F12}~C_{L}~U_{F12}~U_{M}~A_{L}~G_{L}~A_{M}
P-U_{M}~G_{F}~G_{L}C_{F4}~A_{M}G_{L}~U_{M12}G_{F}~U_{M}C_{F4}~G_{L}C_{F4}~U_{M12}U~C_{M4}U_{F}~C_{L4}~U_{F}~U_{M12}~A_{L}~G_{L}~A_{M}
P-U_{M}~G_{F}~G_{L}C_{F4}~A_{M}G_{L}~U_{M12}G_{F}~U_{M12}C_{F4}~G_{L}C_{F4}~U_{M12}U~C_{M4}U_{F12}~C_{L4}~U_{F12}~U_{M12}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U~C_{M}U_{F18}~C_{L}~U_{F18}~U_{M}~A_{L}~G_{L}~A_{M}
P-U_{M}~G_{F}~G_{L}C_{F}~A_{M}G_{L}~U_{M}G_{F}~U_{M18}C_{F}~G_{L}C_{F}~U_{M18}U~C_{M}U_{F18}~C_{L}~U_{F18}~U_{M}~A_{L}~G_{L}~A_{M}
P-U_{M}~G_{F}~G_{L}C_{F}~A_{M}G_{L}~U_{M18}G_{F}~U_{M}C_{F}~G_{L}C_{F}~U_{M18}U~C_{M}U_{F}~C_{L}~U_{F}~U_{M18}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U~C_{M}U_{F18}~C_{L}~U_{F18}~U_{M18}~A_{L}~G_{L}~A_{M}
P-U_{M}~G_{F}~G_{L}C_{F4}~A_{M}G_{L}~U_{M18}G_{F}~U_{M18}C_{F}~G_{L}C_{F}~U_{M18}U~C_{M}U_{F18}~C_{L4}~U_{F18}~U_{M}~A_{L}~G_{L}~A_{M}
P-U_{M}~G_{F}~G_{L}C_{F4}~A_{M}G_{L}~U_{M}G_{F}~U_{M18}C_{F}~G_{L}C_{F4}~U_{M18}U~C_{M4}U_{F18}~C_{L}~U_{F18}~U_{M}~A_{L}~G_{L}~A_{M}
P-U_{M}~G_{F}~G_{L}C_{F4}~A_{M}G_{L}~U_{M18}G_{F}~U_{M}C_{F4}~G_{L}C_{F4}~U_{M18}U~C_{M4}U_{F}~C_{L4}~U_{F}~U_{M18}~A_{L}~G_{L}~A_{M}
P-U_{M}~G_{F}~G_{L}C_{F4}~A_{M}G_{L}~U_{M18}G_{F}~U_{M18}C_{F4}~G_{L}C_{F4}~U_{M18}U~C_{M4}U_{F18}~C_{L4}~U_{F18}~U_{M18}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U_{F}~C_{L}U_{F3}~C_{M}~U_{L}~U_{M}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U~C_{L}U_{F3}~C_{M}~U_{L}~U_{M}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U_{F}~C_{L}U_{F3}~C_{M}~U_{L3}~U_{M}~A_{L}~G_{L}~A_{M}
P-U_{M}~G_{F}~G_{L}C_{F}~A_{M}G_{L}~U_{M}G_{F}~U_{M3}C_{F}~G_{L}C_{F}~U_{M3}U_{F}~C_{L}U_{F3}~C_{M}~U_{L3}~U_{M}~A_{L}~G_{L}~A_{M}
P-U_{M}~G_{F}~G_{L}C_{F}~A_{M}G_{L}~U_{M3}G_{F}~U_{M}C_{F}~G_{L}C_{F}~U_{M3}U_{F}~C_{L}U_{F}~C_{M}~U_{L}~U_{M3}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U_{F3}~C_{L}U_{F3}~C_{M}~U_{L3}~U_{M3}~A_{L}~G_{L}~A_{M}
P-U_{M}~G_{F}~G_{L}C_{F4}~A_{M}G_{L}~U_{M3}G_{F}~U_{M3}C_{F}~G_{L}C_{F}~U_{M3}U_{F}~C_{L}U_{F3}~C_{M4}~U_{L3}~U_{M}~A_{L}~G_{L}~A_{M}
P-U_{M}~G_{F}~G_{L}C_{F4}~A_{M}G_{L}~U_{M}G_{F}~U_{M3}C_{F}~G_{L}C_{F4}~U_{M3}U_{F}~C_{L4}U_{F3}~C_{M}~U_{L3}~U_{M}~A_{L}~G_{L}~A_{M}
P-U_{M}~G_{F}~G_{L}C_{F4}~A_{M}G_{L}~U_{M3}G_{F}~U_{M}C_{F4}~G_{L}C_{F4}~U_{M3}U_{F}~C_{L4}U_{F}~C_{M4}~U_{L}~U_{M3}~A_{L}~G_{L}~A_{M}
P-U_{M}~G_{F}~G_{L}C_{F4}~A_{M}G_{L}~U_{M3}G_{F}~U_{M3}C_{F4}~G_{L}C_{F4}~U_{M3}U_{F3}~C_{L4}U_{F3}~C_{M4}~U_{L3}~U_{M3}~A_{L}~G_{L}~A_{M}
P-U_{M}~G_{F}~G_{L}C_{F}~A_{M}G_{L}~U_{M12}G_{F}~U_{M12}C_{F}~G_{L}C_{F}~U_{M12}U_{F}~C_{L}U_{F12}~C_{M}~U_{L12}~U_{M}~A_{L}~G_{L}~A_{M}
P-U_{M}~G_{F}~G_{L}C_{F}~A_{M}G_{L}~U_{M}G_{F}~U_{M12}C_{F}~G_{L}C_{F}~U_{M12}U_{F}~C_{L}U_{F12}~C_{M}~U_{L12}~U_{M}~A_{L}~G_{L}~A_{M}
P-U_{M}~G_{F}~G_{L}C_{F}~A_{M}G_{L}~U_{M12}G_{F}~U_{M}C_{F}~G_{L}C_{F}~U_{M12}U_{F}~C_{L}U_{F}~C_{M}~U_{L}~U_{M12}~A_{L}~G_{L}~A_{M}
P-U_{M}~G_{F}~G_{L}C_{F}~A_{M}G_{L}~U_{M12}G_{F}~U_{M12}C_{F}~G_{L}C_{F}~U_{M12}U_{F12}~C_{L}U_{F12}~C_{M}~U_{L12}~U_{M12}~A_{L}~G_{L}~A_{M}
P-U_{M}~G_{F}~G_{L}C_{F4}~A_{M}G_{L}~U_{M12}G_{F}~U_{M12}C_{F}~G_{L}C_{F}~U_{M12}U_{F}~C_{L}U_{F12}~C_{M4}~U_{L12}~U_{M}~A_{L}~G_{L}~A_{M}
P-U_{M}~G_{F}~G_{L}C_{F4}~A_{M}G_{L}~U_{M}G_{F}~U_{M12}C_{F}~G_{L}C_{F4}~U_{M12}U_{F}~C_{L4}U_{F12}~C_{M}~U_{L12}~U_{M}~A_{L}~G_{L}~A_{M}
P-U_{M}~G_{F}~G_{L}C_{F4}~A_{M}G_{L}~U_{M12}G_{F}~U_{M}C_{F4}~G_{L}C_{F4}~U_{M12}U_{F}~C_{L4}U_{F}~C_{M4}~U_{L}~U_{M12}~A_{L}~G_{L}~A_{M}
P-U_{M}~G_{F}~G_{L}C_{F4}~A_{M}G_{L}~U_{M12}G_{F}~U_{M12}C_{F4}~G_{L}C_{F4}~U_{M12}U_{F12}~C_{L4}U_{F12}~C_{M4}~U_{L12}~U_{M12}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U_{F}~C_{L}U_{F18}~C_{M}~U_{L18}~U_{M}~A_{L}~G_{L}~A_{M}
P-U_{M}~G_{F}~G_{L}C_{F}~A_{M}G_{L}~U_{M}G_{F}~U_{M18}C_{F}~G_{L}C_{F}~U_{M18}U_{F}~C_{L}U_{F18}~C_{M}~U_{L18}~U_{M}~A_{L}~G_{L}~A_{M}
P-U_{M}~G_{F}~G_{L}C_{F}~A_{M}G_{L}~U_{M18}G_{F}~U_{M}C_{F}~G_{L}C_{F}~U_{M18}U_{F}~C_{L}U_{F}~C_{M}~U_{L}~U_{M18}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U_{F18}~C_{L}U_{F18}~C_{M}~U_{L18}~U_{M18}~A_{L}~G_{L}~A_{M}
P-U_{M}~G_{F}~G_{L}C_{F4}~A_{M}G_{L}~U_{M18}G_{F}~U_{M18}C_{F}~G_{L}C_{F}~U_{M18}U_{F}~C_{L}U_{F18}~C_{M4}~U_{L18}~U_{M}~A_{L}~G_{L}~A_{M}
P-U_{M}~G_{F}~G_{L}C_{F4}~A_{M}G_{L}~U_{M}G_{F}~U_{M18}C_{F}~G_{L}C_{F4}~U_{M18}U_{F}~C_{L4}U_{F18}~C_{M}~U_{L18}~U_{M}~A_{L}~G_{L}~A_{M}
P-U_{M}~G_{F}~G_{L}C_{F4}~A_{M}G_{L}~U_{M18}G_{F}~U_{M}C_{F4}~G_{L}C_{F4}~U_{M18}U_{F}~C_{L4}U_{F}~C_{M4}~U_{L}~U_{M18}~A_{L}~G_{L}~A_{M}
P-U_{M}~G_{F}~G_{L}C_{F4}~A_{M}G_{L}~U_{M18}G_{F}~U_{M18}C_{F4}~G_{L}C_{F4}~U_{M18}U_{F18}~C_{L4}U_{F18}~C_{M4}~U_{L18}~U_{M18}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U~C_{L}U_{F3}~C_{M}~U_{L3}~U_{M}~A_{L}~G_{L}~A_{M}
P-U_{M}~G_{F}~G_{L}C_{F}~A_{M}G_{L}~U_{M}G_{F}~U_{M3}C_{F}~G_{L}C_{F}~U_{M3}U~C_{L}U_{F3}~C_{M}~U_{L3}~U_{M}~A_{L}~G_{L}~A_{M}
P-U_{M}~G_{F}~G_{L}C_{F}~A_{M}G_{L}~U_{M3}G_{F}~U_{M}C_{F}~G_{L}C_{F}~U_{M3}U~C_{L}U_{F}~C_{M}~U_{L}~U_{M3}~A_{L}~G_{L}~A_{M}
P-U_{M}~G_{F}~G_{L}C_{F}~A_{M}G_{L}~U_{M3}G_{F}~U_{M3}C_{F}~G_{L}C_{F}~U_{M3}U~C_{L}U_{F3}~C_{M}~U_{L3}~U_{M3}~A_{L}~G_{L}~A_{M}
P-U_{M}~G_{F}~G_{L}C_{F4}~A_{M}G_{L}~U_{M3}G_{F}~U_{M3}C_{F}~G_{L}C_{F}~U_{M3}U~C_{L}U_{F3}~C_{M4}~U_{L3}~U_{M}~A_{L}~G_{L}~A_{M}
P-U_{M}~G_{F}~G_{L}C_{F4}~A_{M}G_{L}~U_{M}G_{F}~U_{M3}C_{F}~G_{L}C_{F4}~U_{M3}U~C_{L4}U_{F3}~C_{M}~U_{L3}~U_{M}~A_{L}~G_{L}~A_{M}
P-U_{M}~G_{F}~G_{L}C_{F4}~A_{M}G_{L}~U_{M3}G_{F}~U_{M}C_{F4}~G_{L}C_{F4}~U_{M3}U~C_{L4}U_{F}~C_{M4}~U_{L}~U_{M3}~A_{L}~G_{L}~A_{M}
P-U_{M}~G_{F}~G_{L}C_{F4}~A_{M}G_{L}~U_{M3}G_{F}~U_{M3}C_{F4}~G_{L}C_{F4}~U_{M3}U~C_{L4}U_{F3}~C_{M4}~U_{L3}~U_{M3}~A_{L}~G_{L}~A_{M}
P-U_{M}~G_{F}~G_{L}C_{F}~A_{M}G_{L}~U_{M12}G_{F}~U_{M12}C_{F}~G_{L}C_{F}~U_{M12}U~C_{L}U_{F12}~C_{M}~U_{L12}~U_{M}~A_{L}~G_{L}~A_{M}
P-U_{M}~G_{F}~G_{L}C_{F}~A_{M}G_{L}~U_{M}G_{F}~U_{M12}C_{F}~G_{L}C_{F}~U_{M12}U~C_{L}U_{F12}~C_{M}~U_{L12}~U_{M}~A_{L}~G_{L}~A_{M}
P-U_{M}~G_{F}~G_{L}C_{F}~A_{M}G_{L}~U_{M12}G_{F}~U_{M}C_{F}~G_{L}C_{F}~U_{M12}U~C_{L}U_{F}~C_{M}~U_{L}~U_{M12}~A_{L}~G_{L}~A_{M}
P-U_{M}~G_{F}~G_{L}C_{F}~A_{M}G_{L}~U_{M12}G_{F}~U_{M12}C_{F}~G_{L}C_{F}~U_{M12}U~C_{L}U_{F12}~C_{M}~U_{L12}~U_{M12}~A_{L}~G_{L}~A_{M}
P-U_{M}~G_{F}~G_{L}C_{F4}~A_{M}G_{L}~U_{M12}G_{F}~U_{M12}C_{F}~G_{L}C_{F}~U_{M12}U~C_{L}U_{F12}~C_{M4}~U_{L12}~U_{M}~A_{L}~G_{L}~A_{M}
P-U_{M}~G_{F}~G_{L}C_{F4}~A_{M}G_{L}~U_{M}G_{F}~U_{M12}C_{F}~G_{L}C_{F4}~U_{M12}U~C_{L4}U_{F12}~C_{M}~U_{L12}~U_{M}~A_{L}~G_{L}~A_{M}
P-U_{M}~G_{F}~G_{L}C_{F4}~A_{M}G_{L}~U_{M12}G_{F}~U_{M}C_{F4}~G_{L}C_{F4}~U_{M12}U~C_{L4}U_{F}~C_{M4}~U_{L}~U_{M12}~A_{L}~G_{L}~A_{M}
P-U_{M}~G_{F}~G_{L}C_{F4}~A_{M}G_{L}~U_{M12}G_{F}~U_{M12}C_{F4}~G_{L}C_{F4}~U_{M12}U~C_{L4}U_{F12}~C_{M4}~U_{L12}~U_{M12}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U~C_{L}U_{F18}~C_{M}~U_{L18}~U_{M}~A_{L}~G_{L}~A_{M}
P-U_{M}~G_{F}~G_{L}C_{F}~A_{M}G_{L}~U_{M}G_{F}~U_{M18}C_{F}~G_{L}C_{F}~U_{M18}U~C_{L}U_{F18}~C_{M}~U_{L18}~U_{M}~A_{L}~G_{L}~A_{M}
P-U_{M}~G_{F}~G_{L}C_{F}~A_{M}G_{L}~U_{M18}G_{F}~U_{M}C_{F}~G_{L}C_{F}~U_{M18}U~C_{L}U_{F}~C_{M}~U_{L}~U_{M18}~A_{L}~G_{L}~A_{M}
P-U_{M}~G_{F}~G_{L}C_{F}~A_{M}G_{L}~U_{M18}G_{F}~U_{M18}C_{F}~G_{L}C_{F}~U_{M18}U~C_{L}U_{F18}~C_{M}~U_{L18}~U_{M18}~A_{L}~G_{L}~A_{M}
P-U_{M}~G_{F}~G_{L}C_{F4}~A_{M}G_{L}~U_{M18}G_{F}~U_{M18}C_{F}~G_{L}C_{F}~U_{M18}U~C_{L}U_{F18}~C_{M4}~U_{L18}~U_{M}~A_{L}~G_{L}~A_{M}
P-U_{M}~G_{F}~G_{L}C_{F4}~A_{M}G_{L}~U_{M}G_{F}~U_{M18}C_{F}~G_{L}C_{F4}~U_{M18}U~C_{L4}U_{F18}~C_{M}~U_{L18}~U_{M}~A_{L}~G_{L}~A_{M}
P-U_{M}~G_{F}~G_{L}C_{F4}~A_{M}G_{L}~U_{M18}G_{F}~U_{M}C_{F4}~G_{L}C_{F4}~U_{M18}U~C_{L4}U_{F}~C_{M4}~U_{L}~U_{M18}~A_{L}~G_{L}~A_{M}
P-U_{M}~G_{F}~G_{L}C_{F4}~A_{M}G_{L}~U_{M18}G_{F}~U_{M18}C_{F4}~G_{L}C_{F4}~U_{M18}U~C_{L4}U_{F18}~C_{M4}~U_{L18}~U_{M18}~A_{L}~G_{L}~A_{M}
P-U_{M}~G_{L}~G_{M}C_{F}~A_{L}G_{F}~U_{M3}G_{L}~U_{M3}C_{F}~G_{M}C_{L}~U_{M3}U_{F}~C_{L}U_{F3}~C_{M}~U_{L}~U_{M}~A_{L}~G_{L}~A_{M}
P-U_{M}~G_{L}~G_{M}C_{F}~A_{L}G_{F}~U_{M3}G_{L}~U_{M3}C_{F}~G_{M}C_{L}~U_{M3}U~C_{L}U_{F3}~C_{M}~U_{L}~U_{M}~A_{L}~G_{L}~A_{M}
P-U_{M}~G_{L}~G_{M}C_{F}~A_{L}G_{F}~U_{M3}G_{L}~U_{M3}C_{F}~G_{M}C_{L}~U_{M3}U_{F}~C_{L}U_{F3}~C_{M}~U_{L3}~U_{M}~A_{L}~G_{L}~A_{M}
P-U_{M}~G_{L}~G_{M}C_{F}~A_{L}G_{F}~U_{M}G_{L}~U_{M3}C_{F}~G_{M}C_{L}~U_{M3}U_{F}~C_{L}U_{F3}~C_{M}~U_{L3}~U_{M}~A_{L}~G_{L}~A_{M}
P-U_{M}~G_{L}~G_{M}C_{F}~A_{L}G_{F}~U_{M3}G_{L}~U_{M}C_{F}~G_{M}C_{L}~U_{M3}U_{F}~C_{L}U_{F}~C_{M}~U_{L}~U_{M3}~A_{L}~G_{L}~A_{M}
P-U_{M}~G_{L}~G_{M}C_{F}~A_{L}G_{F}~U_{M3}G_{L}~U_{M3}C_{F}~G_{M}C_{L}~U_{M3}U_{F3}~C_{L}U_{F3}~C_{M}~U_{L3}~U_{M3}~A_{L}~G_{L}~A_{M}
P-U_{M}~G_{L}~G_{M}C_{F4}~A_{L}G_{F}~U_{M3}G_{L}~U_{M3}C_{F}~G_{M}C_{L}~U_{M3}U_{F}~C_{L}U_{F3}~C_{M4}~U_{L3}~U_{M}~A_{L}~G_{L}~A_{M}
P-U_{M}~G_{L}~G_{M}C_{F4}~A_{L}G_{F}~U_{M}G_{L}~U_{M3}C_{F}~G_{M}C_{L4}~U_{M3}U_{F}~C_{L4}U_{F3}~C_{M}~U_{L3}~U_{M}~A_{L}~G_{L}~A_{M}
P-U_{M}~G_{L}~G_{M}C_{F4}~A_{L}G_{F}~U_{M3}G_{L}~U_{M}C_{F4}~G_{M}C_{L4}~U_{M3}U_{F}~C_{L4}U_{F}~C_{M4}~U_{L}~U_{M3}~A_{L}~G_{L}~A_{M}
P-U_{M}~G_{L}~G_{M}C_{F4}~A_{L}G_{F}~U_{M3}G_{L}~U_{M3}C_{F4}~G_{M}C_{L4}~U_{M3}U_{F3}~C_{L4}U_{F3}~C_{M4}~U_{L3}~U_{M3}~A_{L}~G_{L}~A_{M}
P-U_{M}~G_{L}~G_{M}C_{F}~A_{L}G_{F}~U_{M12}G_{L}~U_{M12}C_{F}~G_{M}C_{L}~U_{M12}U_{F}~C_{L}U_{F12}~C_{M}~U_{L12}~U_{M}~A_{L}~G_{L}~A_{M}
P-U_{M}~G_{L}~G_{M}C_{F}~A_{L}G_{F}~U_{M}G_{L}~U_{M12}C_{F}~G_{M}C_{L}~U_{M12}U_{F}~C_{L}U_{F12}~C_{M}~U_{L12}~U_{M}~A_{L}~G_{L}~A_{M}
P-U_{M}~G_{L}~G_{M}C_{F}~A_{L}G_{F}~U_{M12}G_{L}~U_{M}C_{F}~G_{M}C_{L}~U_{M12}U_{F}~C_{L}U_{F}~C_{M}~U_{L}~U_{M12}~A_{L}~G_{L}~A_{M}
P-U_{M}~G_{L}~G_{M}C_{F}~A_{L}G_{F}~U_{M12}G_{L}~U_{M12}C_{F}~G_{M}C_{L}~U_{M12}U_{F12}~C_{L}U_{F12}~C_{M}~U_{L12}~U_{M12}~A_{L}~G_{L}~A_{M}
P-U_{M}~G_{L}~G_{M}C_{F4}~A_{L}G_{F}~U_{M12}G_{L}~U_{M12}C_{F}~G_{M}C_{L}~U_{M12}U_{F}~C_{L}U_{F12}~C_{M4}~U_{L12}~U_{M}~A_{L}~G_{L}~A_{M}
P-U_{M}~G_{L}~G_{M}C_{F4}~A_{L}G_{F}~U_{M}G_{L}~U_{M12}C_{F}~G_{M}C_{L4}~U_{M12}U_{F}~C_{L4}U_{F12}~C_{M}~U_{L12}~U_{M}~A_{L}~G_{L}~A_{M}
P-U_{M}~G_{L}~G_{M}C_{F4}~A_{L}G_{F}~U_{M12}G_{L}~U_{M}C_{F4}~G_{M}C_{L4}~U_{M12}U_{F}~C_{L4}U_{F}~C_{M4}~U_{L}~U_{M12}~A_{L}~G_{L}~A_{M}
P-U_{M}~G_{L}~G_{M}C_{F4}~A_{L}G_{F}~U_{M12}G_{L}~U_{M12}C_{F4}~G_{M}C_{L4}~U_{M12}U_{F12}~C_{L4}U_{F12}~C_{M4}~U_{L12}~U_{M12}~A_{L}~G_{L}~A_{M}
P-U_{M}~G_{L}~G_{M}C_{F}~A_{L}G_{F}~U_{M18}G_{L}~U_{M18}C_{F}~G_{M}C_{L}~U_{M18}U_{F}~C_{L}U_{F18}~C_{M}~U_{L18}~U_{M}~A_{L}~G_{L}~A_{M}
P-U_{M}~G_{L}~G_{M}C_{F}~A_{L}G_{F}~U_{M}G_{L}~U_{M18}C_{F}~G_{M}C_{L}~U_{M18}U_{F}~C_{L}U_{F18}~C_{M}~U_{L18}~U_{M}~A_{L}~G_{L}~A_{M}
P-U_{M}~G_{L}~G_{M}C_{F}~A_{L}G_{F}~U_{M18}G_{L}~U_{M}C_{F}~G_{M}C_{L}~U_{M18}U_{F}~C_{L}U_{F}~C_{M}~U_{L}~U_{M18}~A_{L}~G_{L}~A_{M}
P-U_{M}~G_{L}~G_{M}C_{F}~A_{L}G_{F}~U_{M18}G_{L}~U_{M18}C_{F}~G_{M}C_{L}~U_{M18}U_{F18}~C_{L}U_{F18}~C_{M}~U_{L18}~U_{M18}~A_{L}~G_{L}~A_{M}
P-U_{M}~G_{L}~G_{M}C_{F4}~A_{L}G_{F}~U_{M18}G_{L}~U_{M18}C_{F}~G_{M}C_{L}~U_{M18}U_{F}~C_{L}U_{F18}~C_{M4}~U_{L18}~U_{M}~A_{L}~G_{L}~A_{M}
P-U_{M}~G_{L}~G_{M}C_{F4}~A_{L}G_{F}~U_{M}G_{L}~U_{M18}C_{F}~G_{M}C_{L4}~U_{M18}U_{F}~C_{L4}U_{F18}~C_{M}~U_{L18}~U_{M}~A_{L}~G_{L}~A_{M}
P-U_{M}~G_{L}~G_{M}C_{F4}~A_{L}G_{F}~U_{M18}G_{L}~U_{M}C_{F4}~G_{M}C_{L4}~U_{M18}U_{F}~C_{L4}U_{F}~C_{M4}~U_{L}~U_{M18}~A_{L}~G_{L}~A_{M}
P-U_{M}~G_{L}~G_{M}C_{F4}~A_{L}G_{F}~U_{M18}G_{L}~U_{M18}C_{F4}~G_{M}C_{L4}~U_{M18}U_{F18}~C_{L4}U_{F18}~C_{M4}~U_{L18}~U_{M18}~A_{L}~G_{L}~A_{M}
P-U_{M}~G_{L}~G_{M}C_{F}~A_{L}G_{F}~U_{M3}G_{L}~U_{M3}C_{F}~G_{M}C_{L}~U_{M3}U~C_{L}U_{F3}~C_{M}~U_{L3}~U_{M}~A_{L}~G_{L}~A_{M}
P-U_{M}~G_{L}~G_{M}C_{F}~A_{L}G_{F}~U_{M}G_{L}~U_{M3}C_{F}~G_{M}C_{L}~U_{M3}U~C_{L}U_{F3}~C_{M}~U_{L3}~U_{M}~A_{L}~G_{L}~A_{M}
P-U_{M}~G_{L}~G_{M}C_{F}~A_{L}G_{F}~U_{M3}G_{L}~U_{M}C_{F}~G_{M}C_{L}~U_{M3}U~C_{L}U_{F}~C_{M}~U_{L}~U_{M3}~A_{L}~G_{L}~A_{M}
P-U_{M}~G_{L}~G_{M}C_{F}~A_{L}G_{F}~U_{M3}G_{L}~U_{M3}C_{F}~G_{M}C_{L}~U_{M3}U~C_{L}U_{F3}~C_{M}~U_{L3}~U_{M3}~A_{L}~G_{L}~A_{M}
P-U_{M}~G_{L}~G_{M}C_{F4}~A_{L}G_{F}~U_{M3}G_{L}~U_{M3}C_{F}~G_{M}C_{L}~U_{M3}U~C_{L}U_{F3}~C_{M4}~U_{L3}~U_{M}~A_{L}~G_{L}~A_{M}
P-U_{M}~G_{L}~G_{M}C_{F4}~A_{L}G_{F}~U_{M}G_{L}~U_{M3}C_{F}~G_{M}C_{L4}~U_{M3}U~C_{L4}U_{F3}~C_{M}~U_{L3}~U_{M}~A_{L}~G_{L}~A_{M}
P-U_{M}~G_{L}~G_{M}C_{F4}~A_{L}G_{F}~U_{M3}G_{L}~U_{M}C_{F4}~G_{M}C_{L4}~U_{M3}U~C_{L4}U_{F}~C_{M4}~U_{L}~U_{M3}~A_{L}~G_{L}~A_{M}
P-U_{M}~G_{L}~G_{M}C_{F4}~A_{L}G_{F}~U_{M3}G_{L}~U_{M3}C_{F4}~G_{M}C_{L4}~U_{M3}U~C_{L4}U_{F3}~C_{M4}~U_{L3}~U_{M3}~A_{L}~G_{L}~A_{M}
P-U_{M}~G_{L}~G_{M}C_{F}~A_{L}G_{F}~U_{M12}G_{L}~U_{M12}C_{F}~G_{M}C_{L}~U_{M12}U~C_{L}U_{F12}~C_{M}~U_{L12}~U_{M}~A_{L}~G_{L}~A_{M}
P-U_{M}~G_{L}~G_{M}C_{F}~A_{L}G_{F}~U_{M}G_{L}~U_{M12}C_{F}~G_{M}C_{L}~U_{M12}U~C_{L}U_{F12}~C_{M}~U_{L12}~U_{M}~A_{L}~G_{L}~A_{M}
P-U_{M}~G_{L}~G_{M}C_{F}~A_{L}G_{F}~U_{M12}G_{L}~U_{M}C_{F}~G_{M}C_{L}~U_{M12}U~C_{L}U_{F}~C_{M}~U_{L}~U_{M12}~A_{L}~G_{L}~A_{M}
P-U_{M}~G_{L}~G_{M}C_{F}~A_{L}G_{F}~U_{M12}G_{L}~U_{M12}C_{F}~G_{M}C_{L}~U_{M12}U~C_{L}U_{F12}~C_{M}~U_{L12}~U_{M12}~A_{L}~G_{L}~A_{M}
P-U_{M}~G_{L}~G_{M}C_{F4}~A_{L}G_{F}~U_{M12}G_{L}~U_{M12}C_{F}~G_{M}C_{L}~U_{M12}U~C_{L}U_{F12}~C_{M4}~U_{L12}~U_{M}~A_{L}~G_{L}~A_{M}
P-U_{M}~G_{L}~G_{M}C_{F4}~A_{L}G_{F}~U_{M}G_{L}~U_{M12}C_{F}~G_{M}C_{L4}~U_{M12}U~C_{L4}U_{F12}~C_{M}~U_{L12}~U_{M}~A_{L}~G_{L}~A_{M}
P-U_{M}~G_{L}~G_{M}C_{F4}~A_{L}G_{F}~U_{M12}G_{L}~U_{M}C_{F4}~G_{M}C_{L4}~U_{M12}U~C_{L4}U_{F}~C_{M4}~U_{L}~U_{M12}~A_{L}~G_{L}~A_{M}
P-U_{M}~G_{L}~G_{M}C_{F4}~A_{L}G_{F}~U_{M12}G_{L}~U_{M12}C_{F4}~G_{M}C_{L4}~U_{M12}U~C_{L4}U_{F12}~C_{M4}~U_{L12}~U_{M12}~A_{L}~G_{L}~A_{M}
P-U_{M}~G_{L}~G_{M}C_{F}~A_{L}G_{F}~U_{M18}G_{L}~U_{M18}C_{F}~G_{M}C_{L}~U_{M18}U~C_{L}U_{F18}~C_{M}~U_{L18}~U_{M}~A_{L}~G_{L}~A_{M}
P-U_{M}~G_{L}~G_{M}C_{F}~A_{L}G_{F}~U_{M}G_{L}~U_{M18}C_{F}~G_{M}C_{L}~U_{M18}U~C_{L}U_{F18}~C_{M}~U_{L18}~U_{M}~A_{L}~G_{L}~A_{M}
P-U_{M}~G_{L}~G_{M}C_{F}~A_{L}G_{F}~U_{M18}G_{L}~U_{M}C_{F}~G_{M}C_{L}~U_{M18}U~C_{L}U_{F}~C_{M}~U_{F}~U_{L18}~A_{L}~G_{L}~A_{M}
P-U_{M}~G_{L}~G_{M}C_{F}~A_{L}G_{F}~U_{M18}G_{L}~U_{M18}C_{F}~G_{M}C_{L}~U_{M18}U~C_{L}U_{F18}~C_{M}~U_{L18}~U_{M18}~A_{L}~G_{L}~A_{M}
P-U_{M}~G_{L}~G_{M}C_{F4}~A_{L}G_{F}~U_{M18}G_{L}~U_{M18}C_{F}~G_{M}C_{L}~U_{M18}U~C_{L}U_{F18}~C_{M4}~U_{L18}~U_{M}~A_{L}~G_{L}~A_{M}
P-U_{M}~G_{L}~G_{M}C_{F4}~A_{L}G_{F}~U_{M}G_{L}~U_{M18}C_{F}~G_{M}C_{L4}~U_{M18}U~C_{L4}U_{F18}~C_{M}~U_{L18}~U_{M}~A_{L}~G_{L}~A_{M}
P-U_{M}~G_{L}~G_{M}C_{F4}~A_{L}G_{F}~U_{M18}G_{L}~U_{M}C_{F4}~G_{M}C_{L4}~U_{M18}U~C_{L4}U_{F}~C_{M4}~U_{L}~U_{M18}~A_{L}~G_{L}~A_{M}
P-U_{M}~G_{L}~G_{M}C_{F4}~A_{L}G_{F}~U_{M18}G_{L}~U_{M18}C_{F4}~G_{M}C_{L4}~U_{M18}U~C_{L4}U_{F18}~C_{M4}~U_{L18}~U_{M18}~A_{L}~G_{L}~A_{M}

### 2) Modular Design Example Set #2

The 11-mer seed vehicle referred to as sequence no. 1 in Table 3 is used in the ags-MiR compounds illustrated in Table 6B. It has the following sequence:

GUUCUUUCUUU

It has to be used with an 8-mer 5′-end module that includes the seed sequence of human and murine miR-34a-5p. In addition to being one nucleoside shorter on the 3′-end this sequence has an A rather than the U in the 5′-end terminal nucleoside of the 5′end module used in Modular Design Example Set #1 in some of the following examples. This illustrates the fact this position can have any of the bases provided herein.

AGGCAGUG

TABLE 6B
Compositions of Matter Suitable for Administration to a Subject with or
without a Protective Carrier and for Any Other use as Provided for Herein
(5′-3′)

P-A_{M}~G_{F}~G_{M}C_{F4}~A_{M}G_{F}~U_{M}G_{F}~G_{M}U_{F3}~U_{M3}C_{F4}~U_{M3}U_{F}~U_{M3}C_{F}~U_{M3}~U_{F3}~U_{M}~G_{L}~C_{L}~U_{F}
P-A_{M}~G_{F}~G_{M}C_{F4}~A_{M}G_{F}~U_{M}G_{F}~G_{M}U_{F3}~U_{M3}C_{F4}~U_{M3}U_{F}~U_{M3}C_{F4}~U_{M}~U_{F3}~U_{M}~G_{L}~C_{L}~U_{F}
P-A_{M}~G_{F}~G_{M}C_{F4}~A_{M}G_{F}~U_{M3}G_{F}~G_{M}U_{F3}~U_{M3}C_{F4}~U_{M3}U_{F3}~U_{M3}C_{F4}~U_{M}~U_{F3}~U_{M3}~G_{L}~C_{L}~U_{F}
P-A_{M}~G_{F}~G_{M}C_{F4}~A_{M}G_{F}~U_{M}G_{F}~G_{M}U_{F12}~U_{M12}C_{F4}~U_{M12}U_{F}~U_{M12}C_{F}~U_{M12}~U_{F12}~U_{M}~G_{L}~C_{L}~U_{F}
P-A_{M}~G_{F}~G_{M}C_{F4}~A_{M}G_{F}~U_{M}G_{F}~G_{M}U_{F12}~U_{M12}C_{F4}~U_{M12}U_{F}~U_{M12}C_{F4}~U_{M}~U_{F12}~U_{M}~G_{L}~C_{L}~U_{F}
P-A_{M}~G_{F}~G_{M}C_{F4}~A_{M}G_{F}~U_{M12}G_{F}~G_{M}U_{F12}~U_{M12}C_{F4}~U_{M12}U_{F12}~U_{M12}C_{F4}~U_{M}~U_{F12}~U_{M12}~G_{L}~C_{L}~U_{F}
P-A_{M}~G_{F}~G_{M}C_{F4}~A_{M}G_{F}~U_{M}G_{F}~G_{M}U_{F18}~U_{M18}C_{F4}~U_{M18}U_{F}~U_{M18}C_{F}~U_{M18}~U_{F18}~U_{M}~G_{L}~C_{L}~U_{F}
P-A_{M}~G_{F}~G_{M}C_{F4}~A_{M}G_{F}~U_{M}G_{F}~G_{M}U_{F18}~U_{M18}C_{F4}~U_{M18}U_{F}~U_{M18}C_{F4}~U_{M}~U_{F18}~U_{M}~G_{L}~C_{L}~U_{F}
P-A_{M}~G_{F}~G_{M}C_{F4}~A_{M}G_{F}~U_{M18}G_{F}~G_{M}U_{F18}~U_{M18}C_{F4}~U_{M18}U_{F18}~U_{M18}C_{F4}~U_{M}~U_{F18}~U_{M18}~G_{L}~C_{L}~U_{F}
P-A_{M}~G_{F}~G_{M}C_{F4}~A_{M}G_{F}~U_{M}G_{F}~G_{M}U_{F3}~U_{M3}C_{F4}~U_{M3}U~U_{M3}C_{F}~U_{M3}~U_{F3}~U_{M}~G_{L}~C_{L}~U_{F}
P-A_{M}~G_{F}~G_{M}C_{F4}~A_{M}G_{F}~U_{M}G_{F}~G_{M}U_{F3}~U_{M3}C_{F4}~U_{M3}U~U_{M3}C_{F4}~U_{M}~U_{F3}~U_{M}~G_{L}~C_{L}~U_{F}
P-A_{M}~G_{F}~G_{M}C_{F4}~A_{M}G_{F}~U_{M3}G_{F}~G_{M}U_{F3}~U_{M3}C_{F4}~U_{M3}U~U_{M3}C_{F4}~U_{M}~U_{F3}~U_{M3}~G_{L}~C_{L}~U_{F}
P-A_{M}~G_{F}~G_{M}C_{F4}~A_{M}G_{F}~U_{M}G_{F}~G_{M}U_{F12}~U_{M12}C_{F4}~U_{M12}U~U_{M12}C_{F}~U_{M12}~U_{F12}~U_{M}~G_{L}~C_{L}~U_{F}
P-A_{M}~G_{F}~G_{M}C_{F4}~A_{M}G_{F}~U_{M}G_{F}~G_{M}U_{F12}~U_{M12}C_{F4}~U_{M12}U~U_{M12}C_{F4}~U_{M}~U_{F12}~U_{M}~G_{L}~C_{L}~U_{F}
P-A_{M}~G_{F}~G_{M}C_{F4}~A_{M}G_{F}~U_{M12}G_{F}~G_{M}U_{F12}~U_{M12}C_{F4}~U_{M12}U~U_{M12}C_{F4}~U_{M}~U_{F12}~U_{M12}~G_{L}~C_{L}~U_{F}
P-A_{M}~G_{F}~G_{M}C_{F4}~A_{M}G_{F}~U_{M}G_{F}~G_{M}U_{F18}~U_{M18}C_{F4}~U_{M18}U~U_{M18}C_{F}~U_{M18}~U_{F18}~U_{M}~G_{L}~C_{L}~U_{F}
P-A_{M}~G_{F}~G_{M}C_{F4}~A_{M}G_{F}~U_{M}G_{F}~G_{M}U_{F18}~U_{M18}C_{F4}~U_{M18}U~U_{M18}C_{F4}~U_{M}~U_{F18}~U_{M}~G_{L}~C_{L}~U_{F}
P-A_{M}~G_{F}~G_{M}C_{F4}~A_{M}G_{F}~U_{M18}G_{F}~G_{M}U_{F18}~U_{M18}C_{F4}~U_{M18}U~U_{M18}C_{F4}~U_{M}~U_{F18}~U_{M18}~G_{L}~C_{L}~U_{F}
P-A_{M}~G_{F}~G_{L}C_{F}~A_{M}G_{L}~U_{M}G_{F}~G_{L}U_{F}~U_{M}C_{L}~U_{M}U_{F}~U_{L}C_{F}~U_{M}~U_{L}~U_{M}~G_{L}~C_{L}~U_{F}
P-A_{M}~G_{F}~G_{L}C_{F}~A_{M}G_{L}~U_{M}G_{F}~G_{L}U_{F}~U_{M}C_{L}~U_{M}U~U_{L}C_{F}~U_{M}~U_{L}~U_{M}~G_{L}~C_{L}~U_{F}
P-A_{M}~G_{F}~G_{L}C_{F4}~A_{M}G_{L}~U_{M}G_{F}~G_{L}U_{F3}~U_{M3}C_{L4}~U_{M3}U_{F}~U_{L3}C_{F}~U_{M3}~U_{L3}~U_{M}~G_{L}~C_{L}~U_{F}
P-A_{M}~G_{F}~G_{L}C_{F4}~A_{M}G_{L}~U_{M}G_{F}~G_{L}U_{F3}~U_{M3}C_{L4}~U_{M3}U_{F}~U_{L3}C_{F4}~U_{M}~U_{L3}~U_{M}~G_{L}~C_{L}~U_{F}
P-A_{M}~G_{F}~G_{L}C_{F4}~A_{M}G_{L}~U_{M3}G_{F}~G_{L}U_{F3}~U_{M3}C_{L4}~U_{M3}U_{F3}~U_{L3}C_{F4}~U_{M}~U_{L3}~U_{M3}~G_{L}~C_{L}~U_{F}
P-A_{M}~G_{F}~G_{L}C_{F4}~A_{M}G_{L}~U_{M}G_{F}~G_{L}U_{F12}~U_{M12}C_{L4}~U_{M12}U_{F}~U_{L12}C_{F}~U_{M12}~U_{L12}~U_{M}~G_{L}~U_{F}
P-A_{M}~G_{F}~G_{L}C_{F4}~A_{M}G_{L}~U_{M}G_{F}~G_{L}U_{F12}~U_{M12}C_{L4}~U_{M12}U_{F}~U_{L12}C_{F4}~U_{M}~U_{L12}~U_{M}~G_{L}~C_{L}~U_{F}
P-A_{M}~G_{F}~G_{L}C_{F4}~A_{M}G_{L}~U_{M12}G_{F}~G_{L}U_{F12}~U_{M12}C_{L4}~U_{M12}U_{F12}~U_{L12}C_{F4}~U_{M}~U_{L12}~U_{M12}~G_{L}~C_{L}~U_{F}
P-A_{M}~Gᵣ~G_{L}C_{F4}~A_{M}G_{L}~U_{M}G_{F}~G_{L}U_{F18}~U_{M18}C_{L4}~U_{M18}U_{F}~U_{L18}C_{F}~U_{M18}~U_{L18}~U_{M}~G_{L}~C_{L}~U_{F}
P-A_{M}~G_{F}~G_{L}C_{F4}~A_{M}G_{L}~U_{M}G_{F}~G_{L}U_{F18}~U_{M18}C_{L4}~U_{M18}U_{F}~U_{L18}C_{F4}~U_{M}~U_{L18}~U_{M}~G_{L}~C_{L}~U_{F}
P-A_{M}~G_{F}~G_{L}C_{F4}~A_{M}G_{L}~U_{M18}G_{F}~G_{L}U_{F18}~U_{M18}C_{L4}~U_{M18}U_{F18}~U_{L18}C_{F4}~U_{M}~U_{L18}~U_{M18}~G_{L}~C_{L}~U_{F}
P-A_{M}~G_{F}~G_{L}C_{F4}~A_{M}G_{L}~U_{M}G_{F}~G_{L}U_{F3}~U_{M3}C_{L4}~U_{M3}U~U_{L3}C_{F}~U_{M3}~U_{L3}~U_{M}~G_{L}~C_{L}~U_{F}
P-A_{M}~G_{F}~G_{L}C_{F4}~A_{M}G_{L}~U_{M}G_{F}~G_{L}U_{F3}~U_{M3}C_{L4}~U_{M3}U~U_{L3}C_{F4}~U_{M}~U_{L3}~U_{M}~G_{L}~C_{L}~U_{F}
P-A_{M}~G_{F}~G_{L}C_{F4}~A_{M}G_{L}~U_{M3}G_{F}~G_{L}U_{F3}~U_{M3}C_{L4}~U_{M3}U~U_{L3}C_{F4}~U_{M}~U_{L3}~U_{M3}~G_{L}~C_{L}~U_{F}
P-A_{M}~G_{F}~G_{L}C_{F4}~A_{M}G_{L}~U_{M}G_{F}~G_{L}U_{F12}~U_{M12}C_{L4}~U_{M12}U~U_{L12}C_{F}~U_{M12}~U_{L12}~U_{M}~G_{L}~C_{L}~U_{F}
P-A_{M}~G_{F}~G_{L}C_{F4}~A_{M}G_{L}~U_{M}G_{F}~G_{L}U_{F12}~U_{M12}C_{L4}~U_{M12}U~U_{L12}C_{F4}~U_{M}~U_{L12}~U_{M}~G_{L}~C_{L}~U_{F}
P-A_{M}~G_{F}~G_{L}C_{F4}~A_{M}G_{L}~U_{M12}G_{F}~G_{L}U_{F12}~U_{M12}C_{L4}~U_{M12}U~U_{L12}C_{F4}~U_{M}~U_{L12}~U_{M12}~G_{L}~C_{L}~U_{F}
P-A_{M}~G_{F}~G_{L}C_{F4}~A_{M}G_{L}~U_{M}G_{F}~G_{L}U_{F18}~U_{M18}C_{L4}~U_{M18}U~U_{L18}C_{F}~U_{M18}~U_{L18}~U_{M}~G_{L}~C_{L}~U_{F}
P-A_{M}~G_{F}~G_{L}C_{F4}~A_{M}G_{L}~U_{M}G_{F}~G_{L}U_{F18}~U_{M18}C_{L4}~U_{M18}U~U_{L18}C_{F4}~U_{M}~U_{L18}~U_{M}~G_{L}~C_{L}~U_{F}
P-A_{M}~G_{F}~G_{L}C_{F4}~A_{M}G_{L}~U_{M18}G_{F}~G_{L}U_{F18}~U_{M18}C_{L4}~U_{M18}U~U_{L18}C_{F4}~U_{M}~U_{L18}~U_{M18}~G_{L}~C_{L}~U_{F}
P-A_{M}~G_{F}~G_{L}C_{F}~A_{L}G_{F}~U_{M}G_{L}~G_{M}U_{F}~U_{L}C_{F}~U_{M}U_{F}~U_{M}C_{F}~U_{L}~U_{F}~U_{M}~G_{L}~C_{L}~U_{F}
P-A_{M}~G_{F}~G_{L}C_{F}~A_{L}G_{F}~U_{M}G_{L}~G_{M}U_{F}~U_{L}C_{F}~U_{M}U~U_{M}C_{F}~U_{L}~U_{F}~U_{M}~G_{L}~C_{L}~U_{F}
P-A_{M}~G_{F}~G_{L}C_{F4}~A_{L}G_{F}~U_{M}G_{L}~G_{M}U_{F3}~U_{L3}C_{F4}~U_{M3}U_{F}~U_{M3}C_{F}~U_{L3}~U_{F3}~U_{M}~G_{L}~C_{L}~U_{F}
P-A_{M}~G_{F}~G_{L}C_{F4}~A_{L}G_{F}~U_{M}G_{L}~G_{M}U_{F3}~U_{L3}C_{F4}~U_{M3}U_{F}~U_{M3}C_{F4}~U_{L}~U_{F3}~U_{M}~G_{L}~C_{L}~U_{F}
P-A_{M}~G_{F}~G_{L}C_{F4}~A_{L}G_{F}~U_{M3}G_{L}~G_{M}U_{F3}~U_{L3}C_{F4}~U_{M3}U_{F3}~U_{M3}C_{F4}~U_{L}~U_{F3}~U_{M3}~G_{L}~C_{L}~U_{F}
P-A_{M}~G_{F}~G_{L}C_{F4}~A_{L}G_{F}~U_{M}G_{L}~G_{M}U_{F12}~U_{L12}C_{F4}~U_{M12}U_{F}~U_{M12}C_{F}~U_{L12}~U_{F12}~U_{M}~G_{L}~C_{L}~U_{F}
P-A_{M}~G_{F}~G_{L}C_{F4}~A_{L}G_{F}~U_{M}G_{L}~G_{M}U_{F12}~U_{L12}C_{F4}~U_{M12}U_{F}~U_{M12}C_{F4}~U_{L}~U_{F12}~U_{M}~G_{L}~C_{L}~U_{F}
P-A_{M}~G_{F}~G_{L}C_{F4}~A_{L}G_{F}~U_{M12}G_{L}~G_{M}U_{F12}~U_{L12}C_{F4}~U_{M12}U_{F12}~U_{M12}C_{F4}~U_{L}~U_{F12}~U_{M12}~G_{L}~C_{L}~U_{F}
P-A_{M}~G_{F}~G_{L}C_{F4}~A_{L}G_{F}~U_{M}G_{L}~G_{M}U_{F18}~U_{L18}C_{F4}~U_{M18}U_{F}~U_{M18}C_{F}~U_{L18}~U_{F18}~U_{M}~G_{L}~C_{L}~U_{F}
P-A_{M}~G_{F}~G_{L}C_{F4}~A_{L}G_{F}~U_{M}G_{L}~G_{M}U_{F18}~U_{L18}C_{F4}~U_{M18}U_{F}~U_{M18}C_{F4}~U_{L}~U_{F18}~U_{M}~G_{L}~C_{L}~U_{F}
P-A_{M}~G_{F}~G_{L}C_{F4}~A_{L}G_{F}~U_{M18}G_{L}~G_{M}U_{F18}~U_{L18}C_{F4}~U_{M18}U_{F18}~U_{M18}C_{F4}~U_{L}~U_{F18}~U_{M18}~G_{L}~C_{L}~U_{F}
P-A_{M}~G_{F}~G_{L}C_{F4}~A_{L}G_{F}~U_{M}G_{L}~G_{M}U_{F3}~U_{L3}C_{F4}~U_{M3}U~U_{M3}C_{F}~U_{L3}~U_{F3}~U_{M}~G_{L}~C_{L}~U_{F}
P-A_{M}~G_{F}~G_{L}C_{F4}~A_{L}G_{F}~U_{M}G_{L}~G_{M}U_{F3}~U_{L3}C_{F4}~U_{M3}U~U_{M3}C_{F4}~U_{L}~U_{F3}~U_{M}~G_{L}~C_{L}~U_{F}
P-A_{M}~G_{F}~G_{L}C_{F4}~A_{L}G_{F}~U_{M3}G_{L}~G_{M}U_{F3}~U_{L3}C_{F4}~U_{M3}U~U_{M3}C_{F4}~U_{L}~U_{F3}~U_{M3}~G_{L}~C_{L}~U_{F}
P-A_{M}~G_{F}~G_{L}C_{F4}~A_{L}G_{F}~U_{M}G_{L}~G_{M}U_{F12}~U_{L12}C_{F4}~U_{M12}U~U_{M12}C_{F}~U_{L12}~U_{F12}~U_{M}~G_{L}~C_{L}~U_{F}
P-A_{M}~G_{F}~G_{L}C_{F4}~A_{L}G_{F}~U_{M}G_{L}~G_{M}U_{F12}~U_{L12}C_{F4}~U_{M12}U~U_{M12}C_{F4}~U_{L}~U_{F12}~U_{M}~G_{L}~C_{L}~U_{F}
P-A_{M}~G_{F}~G_{L}C_{F4}~A_{L}G_{F}~U_{M12}G_{L}~G_{M}U_{F12}~U_{L12}C_{F4}~U_{M12}U~U_{M12}C_{F4}~U_{L}~U_{F12}~U_{M12}~G_{L}~C_{L}~U_{F}
P-A_{M}~G_{F}~G_{L}C_{F4}~A_{L}G_{F}~U_{M}G_{L}~G_{M}U_{F18}~U_{L18}C_{F4}~U_{M18}U~U_{M18}C_{F}~U_{L18}~U_{F18}~U_{M}~G_{L}~C_{L}~U_{F}
P-A_{M}~G_{F}~G_{L}C_{F4}~A_{L}G_{F}~U_{M}G_{L}~G_{M}U_{F18}~U_{L18}C_{F4}~U_{M18}U~U_{M18}C_{F4}~U_{L}~U_{F18}~U_{M}~G_{L}~C_{L}~U_{F}
P-A_{M}~G_{F}~G_{L}C_{F4}~A_{L}G_{F}~U_{M18}G_{L}~G_{M}U_{F18}~U_{L18}C_{F4}~U_{M18}U~U_{M18}C_{F4}~U_{L}~U_{F18}~U_{M18}~G_{L}~C_{L}~U_{F}
P-A_{M}~G_{L}~G_{M}C_{F}~A_{M}G_{L}~U_{M}G_{F}~G_{L}U_{F}~U_{M}C_{F}~U_{L}U_{F}~U_{M}C_{F}~U_{L}~U_{F}~U_{M}~G_{L}~C_{L}~U_{F}
P-A_{M}~G_{L}~G_{M}C_{F}~A_{M}G_{L}~U_{M}G_{F}~G_{L}U_{F}~U_{M}C_{F}~U_{L}U~U_{M}C_{F}~U_{L}~U_{F}~U_{M}~G_{L}~C_{L}~U_{F}
P-A_{M}~G_{L}~G_{M}C_{F4}~A_{M}G_{L}~U_{M}G_{F}~G_{L}U_{F3}~U_{M3}C_{F4}~U_{L3}U_{F}~U_{M3}C_{F}~U_{L3}~U_{F3}~U_{M}~G_{L}~C_{L}~U_{F}
P-A_{M}~G_{L}~G_{M}C_{F4}~A_{M}G_{L}~U_{M}G_{F}~G_{L}U_{F3}~U_{M3}C_{F4}~U_{L3}U_{F}~U_{M3}C_{F4}~U_{L}~U_{F3}~U_{M}~G_{L}~C_{L}~U_{F}
P-A_{M}~G_{L}~G_{M}C_{F4}~A_{M}G_{L}~U_{M3}G_{F}~G_{L}U_{F3}~U_{M3}C_{F4}~U_{L3}U_{F3}~U_{M3}C_{F4}~U_{L}~U_{F3}~U_{M3}~G_{L}~C_{L}~U_{F}
P-A_{M}~G_{L}~G_{M}C_{F4}~A_{M}G_{L}~U_{M}G_{F}~G_{L}U_{F12}~U_{M12}C_{F4}~U_{L12}U_{F}~U_{M12}C_{F}~U_{L12}~U_{F12}~U_{M}~G_{L}~C_{L}~U_{F}
P-A_{M}~G_{L}~G_{M}C_{F4}~A_{M}G_{L}~U_{M}G_{F}~G_{L}U_{F12}~U_{M12}C_{F4}~U_{L12}U_{F}~U_{M12}C_{F4}~U_{L}~U_{F12}~U_{M}~G_{L}~C_{L}~U_{F}
P-A_{M}~G_{L}~G_{M}C_{F4}~A_{M}G_{L}~U_{M12}G_{F}~G_{L}U_{F12}~U_{M12}C_{F4}~U_{L12}U_{F12}~U_{M12}C_{F4}~U_{L}~U_{F12}~U_{M12}~G_{L}~C_{L}~U_{F}
P-A_{M}~G_{L}~G_{M}C_{F4}~A_{M}G_{L}~U_{M}G_{F}~G_{L}U_{F18}~U_{M18}C_{F4}~U_{L18}U_{F}~U_{M18}C_{F}~U_{L18}~U_{F18}~U_{M}~G_{L}~C_{L}~U_{F}
P-A_{M}~G_{L}~G_{M}C_{F4}~A_{M}G_{L}~U_{M}G_{F}~G_{L}U_{F18}~U_{M18}C_{F4}~U_{L18}U_{F}~U_{M18}C_{F4}~U_{L}~U_{F18}~U_{M}~G_{L}~C_{L}~U_{F}
P-A_{M}~G_{L}~G_{M}C_{F4}~A_{M}G_{L}~U_{M18}G_{F}~G_{L}U_{F18}~U_{M18}C_{F4}~U_{L18}U_{F18}~U_{M18}C_{F4}~U_{M}~U_{F18}~U_{M18}~G_{L}~C_{L}~U_{F}
P-A_{M}~G_{L}~G_{M}C_{F4}~A_{M}G_{L}~U_{M}G_{F}~G_{L}U_{F3}~U_{M3}C_{F4}~U_{L3}U~U_{M3}C_{F}~U_{L3}~U_{F3}~U_{M}~G_{L}~C_{L}~U_{F}
P-A_{M}~G_{L}~G_{M}C_{F4}~A_{M}G_{L}~U_{M}G_{F}~G_{L}U_{F3}~U_{M3}C_{F4}~U_{L3}U~U_{M3}C_{F4}~U_{L}~U_{F3}~U_{M}~G_{L}~C_{L}~U_{F}
P-A_{M}~G_{M}C_{F4}~A_{M}G_{L}~U_{M3}G_{F}~G_{L}U_{F3}~U_{M3}C_{F4}~U_{L3}U~U_{M3}C_{F4}~U_{L}~U_{F3}~U_{M3}~G_{L}~C_{L}~U_{F}
P-A_{M}~G_{L}~G_{M}C_{F4}~A_{M}G_{L}~U_{M}G_{F}~G_{L}U_{F12}~U_{M12}C_{F4}~U_{L12}U~U_{M12}C_{F}~U_{L12}~U_{F12}~U_{M}~G_{L}~C_{L}~U_{F}
P-A_{M}~G_{L}~G_{M}C_{F4}~A_{M}G_{L}~U_{M}G_{F}~G_{L}U_{F12}~U_{M12}C_{F4}~U_{L12}U~U_{M12}C_{F4}~U_{L}~U_{F12}~U_{M}~G_{L}~C_{L}~U_{F}
P-A_{M}~G_{L}~G_{M}C_{F4}~A_{M}G_{L}~U_{M12}G_{F}~G_{L}U_{F12}~U_{M12}C_{F4}~U_{L12}U~U_{M12}C_{F4}~U_{L}~U_{F12}~U_{M12}~G_{L}~C_{L}~U_{F}
P-A_{M}~G_{L}~G_{M}C_{F4}~A_{M}G_{L}~U_{M}G_{F}~G_{L}U_{F18}~U_{M18}C_{F4}~U_{L18}U~U_{M18}C_{F}~U_{L18}~U_{F18}~U_{M}~G_{L}~C_{L}~U_{F}
P-A_{M}~G_{L}~G_{M}C_{F4}~A_{M}G_{L}~U_{M}G_{F}~G_{L}U_{F18}~U_{M18}C_{F4}~U_{L18}U~U_{M18}C_{F4}~U_{L}~U_{F18}~U_{M}~G_{L}~C_{L}~U_{F}
P-A_{M}~G_{L}~G_{M}C_{F4}~A_{M}G_{L}~U_{M18}G_{F}~G_{L}U_{F18}~U_{M18}C_{F4}~U_{L18}U~U_{M18}C_{F4}~U_{L}~U_{F18}~U_{M18}~G_{L}~C_{L}~U_{F}

### Table 7

### Examples of Murine/Human MiR-34a-5P ags-IMIR Compositions

A. Murine/Human miR-34a-5p Sequence and Antisense Sequence:

The miRBase accession numbers for murine and human miR-34a-5p are MI0000584 and MI0000268. The guide strand from each species has the same sequence. The sequence minus the overhang is as follows:

UGGCAGUGUCUUAGCUGGUUGU

Hence the antisense sequence is as follows:

ACAACCAGCUAAGACACUGCCA

Any of the 5′-end nucleosides (P-A_{F}) shown in the examples of agsRNAi compounds in Table 7A or 7B can be replaced with any of those provided for herein including one of the 5′end nucleosides selected from the group consisting of P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-A_{M}, P-A_{L}, P-A_{Z}, P-A_{H}, P-A_{B}, P-A_{K}, P-A_{Q}, P-A_{S}, P-A_{T}, P-A_{W}, P-A_{V}, P-A_{O}, P-T_{L}, P-T_{H}, 5′-VP-A_{Z}, 5′-VP-A_{K}, 5′-VP-A_{Q}, 5′-VP-A_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 7A or 7B with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the nucleosides in Tables 7A or 7B with an L modification found in the overhang precursor can have the L replaced with any of those units and linkages provided for herein for overhang precursors including one or more of the sugar, sugar analogs or sugar substitutes independently selected from the group consisting of: F, J, W, V, Y, T, TL and I.

Any of the compounds in Table 7A and 7B can any of those units and linkages provided for herein for overhang precursors.

B. Examples of agsRNAi Compounds:

TABLE 7A
Compositions of Matter Suitable for Administration to a Subject
with or without a Protective Carrier and for Any Other use as
Provided for Herein (5′-3′)

P-A_{F}~C_{M}~A_{F}A_{M}~C_{F4}C_{M4}~A_{F}G_{F}~C_{M}U_{F}~A_{M}A_{F}~G_{M}A~C_{M4}~A_{F}~C_{M4}~U_{F}~G_{M}~A_{F}~G_{M}
P-A_{F}~C_{M}~A_{F}~A_{M}C_{F4}~C_{M4}A_{F}~G_{F}C_{M}~U_{F}A_{M}~A_{F}G_{M}~AC_{M4}~A_{F}~C_{M4}~U_{F}~G_{M}~A_{F}~G_{M}
P-A_{F}~C_{M}~A_{F}A_{M}~C_{F4}C_{M4}~A_{F}G_{F}~C_{M}U_{F}~A_{M}A_{F}~G_{M}A~C_{M4}~A_{F}~C_{M4}~U_{F3}~G_{M}~A_{F}~G_{M}
P-A_{F}~C_{M}~A_{F}~A_{M}C_{F4}~C_{M4}A_{F}~G_{F}C_{M}~U_{F}A_{M}~A_{F}G_{M}~AC_{M4}~A_{F}~C_{M4}~U_{F3}~G_{M}~A_{F}~G_{M}
P-A_{F}~C_{M}~A_{F}A_{M}~C_{F4}C_{M4}~A_{F}G_{F}~C_{M4}U_{F}~A_{M}A_{F}~G_{M}A~C_{M4}~A_{F}~C_{M4}~U_{F3}~G_{M}~A_{F}~G_{M}
P-A_{F}~C_{M}~A_{F}~A_{M}C_{F4}~C_{M4}A_{F}~G_{F}C_{M4}~U_{F}A_{M}~A_{F}G_{M}~AC_{M4}~A_{F}~C_{M4}~U_{F3}~G_{M}~A_{F}~G_{M}
P-A_{F}~C_{M}~A_{F}A_{M}~C_{F4}C_{M4}~A_{F}G_{F}~C_{M}U_{F3}~A_{M}A_{F}~G_{M}A~C_{M4}~A_{F}~C_{M4}~U_{F3}~G_{M}~A_{F}~G_{M}
P-A_{F}~C_{M}~A_{F}~A_{M}C_{F4}~C_{M4}A_{F}~G_{F}C_{M}~U_{F3}A_{M}~A_{F}G_{M}~AC_{M4}~A_{F}~C_{M4}~U_{F3}~G_{M}~A_{F}~G_{M}
P-A_{F}~C_{M}~A_{F}A_{M}~C_{F4}C_{M4}~A_{F}G_{F}~C_{M4}U_{F3}~A_{M}A_{F}~G_{M}A~C_{M4}~A_{F}~C_{M4}~U_{F3}~G_{M}~A_{F}~G_{M}
P-A_{F}~C_{M}~A_{F}~A_{M}C_{F4}~C_{M4}A_{F}~G_{F}C_{M4}~U_{F3}A_{M}~A_{F}G_{M}~AC_{M4}~A_{F}~C_{M4}~U_{F3}~G_{M}~A_{F}~G_{M}
P-A_{F}~C_{M}~A_{F2}A_{M}~C_{F4}C_{M4}~A_{F}G_{F}~C_{M}U_{F}~A_{M}A_{F}~G_{M}A~C_{M4}~A_{F}~C_{M4}~U_{F}~G_{M}~A_{F}~G_{M}
P-A_{F}~C_{M}~A_{F}~A_{M2}C_{F4}~C_{M4}A_{F}~G_{F}C_{M}~U_{F}A_{M}~A_{F}G_{M}~AC_{M4}~A_{F}~C_{M4}~U_{F}~G_{M}~A_{F}~G_{M}
P-A_{F}~C_{M}~A_{F2}A_{M}~C_{F4}C_{M4}~A_{F}G_{F}~C_{M}U_{F}~A_{M}A_{F}~G_{M}A~C_{M4}~A_{F}~C_{M4}~U_{F3}~G_{M}~A_{F}~G_{M}
P-A_{F}~C_{M}~A_{F}~A_{M2}C_{F4}~C_{M4}A_{F}~G_{F}C_{M}~U_{F}A_{M}~A_{F}G_{M}~AC_{M4}~A_{F}~C_{M4}~U_{F3}~G_{M}~A_{F}~G_{M}
P-A_{F}~C_{M}~A_{F2}A_{M}~C_{F4}C_{M4}~A_{F}G_{F}~C_{M4}U_{F}~A_{M}A_{F}~G_{M}A~C_{M4}~A_{F}~C_{M4}~U_{F3}~G_{M}~A_{F}~G_{M}
P-A_{F}~C_{M}~A_{F}~A_{M2}C_{F4}~C_{M4}A_{F}~G_{F}C_{M4}~U_{F}A_{M}~A_{F}G_{M}~AC_{M4}~A_{F}~C_{M4}~U_{F3}~G_{M}~A_{F}~G_{M}
P-A_{F}~C_{M}~A_{F2}A_{M}~C_{F4}C_{M4}~A_{F}G_{F}~C_{M}U_{F3}~A_{M}A_{F}~G_{M}A~C_{M4}~A_{F}~C_{M4}~U_{F3}~G_{M}~A_{F}~G_{M}
P-A_{F}~C_{M}~A_{F}~A_{M2}C_{F4}~C_{M4}A_{F}~G_{F}C_{M}~U_{F3}A_{M}~A_{F}G_{M}~AC_{M4}~A_{F}~C_{M4}~U_{F3}~G_{M}~A_{F}~G_{M}
P-A_{F}~C_{M}~A_{F2}A_{M}~C_{F4}C_{M4}~A_{F}G_{F}~C_{M4}U_{F3}~A_{M}A_{F}~G_{M}A~C_{M4}~A_{F}~C_{M4}~U_{F3}~G_{M}~A_{F}~G_{M}
P-A_{F}~C_{M}~A_{F}~A_{M2}C_{F4}~C_{M4}A_{F}~G_{F}C_{M4}~U_{F3}A_{M}~A_{F}G_{M}~AC_{M4}~A_{F}~C_{M4}~U_{F3}~G_{M}~A_{F}~G_{M}
P-A_{F}~C_{M}~A_{L}~A_{M}~C_{F}C_{M}~A_{L}G_{F}~C_{M}U_{F}~A_{F}A_{L}~G_{M}A~C_{M}~A_{F}~C_{L}~U_{F}~G_{M}~A_{F}~G_{M}
P-A_{F}~C_{M}~A_{L}~A_{M}C_{F4}~C_{M4}A_{L}~G_{F}C_{M}~U_{F}A_{L}~A_{F}G_{M}~AC_{M4}~A_{F}~C_{L4}~U_{F}~G_{M}~A_{F}~G_{M}
P-A_{F}~C_{M}~A_{L}A_{M}~C_{F4}C_{M4}~A_{L}G_{F}~C_{M}U_{F}~A_{L}A_{F}~G_{M}A~C_{M4}~A_{F}~C_{L4}~U_{F3}~G_{M}~A_{F}~G_{M}
P-A_{F}~C_{M}~A_{L}~A_{M}C_{F4}~C_{M4}A_{L}~G_{F}C_{M}~U_{F}A_{L}~A_{F}G_{M}~AC_{M4}~A_{F}~C_{L4}~U_{F3}~G_{M}~A_{F}~G_{M}
P-A_{F}~C_{M}~A_{L}A_{M}~C_{F4}C_{M4}~A_{L}G_{F}~C_{M4}U_{F}~A_{L}A_{F}~G_{M}A~C_{M4}~A_{F}~C_{L4}~U_{F3}~G_{M}~A_{F}~G_{M}
P-A_{F}~C_{M}~A_{L}~A_{M}C_{F4}~C_{M4}A_{L}~G_{F}C_{M4}~U_{F}A_{L}~A_{F}G_{M}~AC_{M4}~A_{F}~C_{L4}~U_{F3}~G_{M}~A_{F}~G_{M}
P-A_{F}~C_{M}~A_{L}A_{M}~C_{F4}C_{M4}~A_{L}G_{F}~C_{M}U_{F3}~A_{L}A_{F}~G_{M}A~C_{M4}~A_{F}~C_{L4}~U_{F3}~G_{M}~A_{F}~G_{M}
P-A_{F}~C_{M}~A_{L}~A_{M}C_{F4}~C_{M4}A_{L}~G_{F}C_{M}~U_{F3}A_{L}~A_{F}G_{M}~AC_{M4}~A_{F}~C_{L4}~U_{F3}~G_{M}~A_{F}~G_{M}
P-A_{F}~C_{M}~A_{L}A_{M}~C_{F4}C_{M4}~A_{L}G_{F}~C_{M4}U_{F3}~A_{L}A_{F}~G_{M}A~C_{M4}~A_{F}~C_{L4}~U_{F3}~G_{M}~A_{F}~G_{M}
P-A_{F}~C_{M}~A_{L}~A_{M}C_{F4}~C_{M4}A_{L}~G_{F}C_{M4}~U_{F3}A_{L}~A_{F}G_{M}~AC_{M4}~A_{F}~C_{L4}~U_{F3}~G_{M}~A_{F}~G_{M}
P-A_{F}~C_{M}~A_{L2}A_{M}~C_{F4}C_{M4}~A_{L}G_{F}~C_{M}U_{F}~A_{L}A_{F}~G_{M}A~C_{M4}~A_{F}~C_{L4}~U_{F}~G_{M}~A_{F}~G_{M}
P-A_{F}~C_{M}~A_{L}~A_{M2}C_{F4}~C_{M4}A_{L}~G_{F}C_{M}~U_{F}A_{L}~A_{F}G_{M}~AC_{M4}~A_{F}~C_{L4}~U_{F}~G_{M}~A_{F}~G_{M}
P-A_{F}~C_{M}~A_{L2}A_{M}~C_{F4}C_{M4}~A_{L}G_{F}~C_{M}U_{F}~A_{L}A_{F}~G_{M}A~C_{M4}~A_{F}~C_{L4}~U_{F3}~G_{M}~A_{F}~G_{M}
P-A_{F}~C_{M}~A_{L}~A_{M2}C_{F4}~C_{M4}A_{L}~G_{F}C_{M}~U_{F}A_{L}~A_{F}G_{M}~AC_{M4}~A_{F}~C_{L4}~U_{F3}~G_{M}~A_{F}~G_{M}
P-A_{F}~C_{M}~A_{L2}A_{M}~C_{F4}C_{M4}~A_{L}G_{F}~C_{M4}U_{F}~A_{L}A_{F}~G_{M}A~C_{M4}~A_{F}~C_{L4}~U_{F3}~G_{M}~A_{F}~G_{M}
P-A_{F}~C_{M}~A_{L}~A_{M2}C_{F4}~C_{M4}A_{L}~G_{F}C_{M4}~U_{F}A_{L}~A_{F}G_{M}~AC_{M4}~A_{F}~C_{L4}~U_{F3}~G_{M}~A_{F}~G_{M}
P-A_{F}~C_{M}~A_{L2}A_{M}~C_{F4}C_{M4}~A_{L}G_{F}~C_{M}U_{F3}~A_{L}A_{F}~G_{M}A~C_{M4}~A_{F}~C_{L4}~U_{F3}~G_{M}~A_{F}~G_{M}
P-A_{F}~C_{M}~A_{L}~A_{M2}C_{F4}~C_{M4}A_{L}~G_{F}C_{M}~U_{F3}A_{L}~A_{F}G_{M}~AC_{M4}~A_{F}~C_{L4}~U_{F3}~G_{M}~A_{F}~G_{M}
P-A_{F}~C_{M}~A_{L2}A_{M}~C_{F4}C_{M4}~A_{L}G_{F}~C_{M4}U_{F3}~A_{L}A_{F}~G_{M}A~C_{M4}~A_{F}~C_{L4}~U_{F3}~G_{M}~A_{F}~G_{M}
P-A_{F}~C_{M}~A_{L}~A_{M2}C_{F4}~C_{M4}A_{L}~G_{F}C_{M4}~U_{F3}A_{L}~A_{F}G_{M}~AC_{M4}~A_{F}~C_{L4}~U_{F3}~G_{M}~A_{F}~G_{M}
P-A_{F}~C_{M}~A_{L}A_{M}~C_{F}C_{M}~A_{L}G_{F}~C_{M}U_{F}~A_{F}A_{L}~G_{M}A~C_{M}~A_{F}~C_{L}~U_{F}~G_{M}~A_{L}~G_{L}~G_{M}
P-A_{F}~C_{M}~A_{L}~A_{M}C_{F4}~C_{M4}A_{L}~G_{F}C_{M}~U_{F}A_{L}~A_{F}G_{M}~AC_{M4}~A_{F}~C_{L4}~U_{F}~G_{M}~A_{L}~G_{L}~G_{M}
P-A_{F}~C_{M}~A_{L}A_{M}~C_{F4}C_{M4}~A_{L}G_{F}~C_{M}U_{F}~A_{L}A_{F}~G_{M}A~C_{M4}~A_{F}~C_{L4}~U_{F3}~G_{M}~A_{L}~G_{L}~G_{M}
P-A_{F}~C_{M}~A_{L}~A_{M}C_{F4}~C_{M4}A_{L}~G_{F}C_{M}~U_{F}A_{L}~A_{F}G_{M}~AC_{M4}~A_{F}~C_{L4}~U_{F3}~G_{M}~A_{L}~G_{L}~G_{M}
P-A_{F}~C_{M}~A_{L}A_{M}~C_{F4}C_{M4}~A_{L}G_{F}~C_{M4}U_{F}~A_{L}A_{F}~G_{M}A~C_{M4}~A_{F}~C_{L4}~U_{F3}~G_{M}~A_{L}~G_{L}~G_{M}
P-A_{F}~C_{M}~A_{L}~A_{M}C_{F4}~C_{M4}A_{L}~G_{F}C_{M4}~U_{F}A_{L}~A_{F}G_{M}~AC_{M4}~A_{F}~C_{L4}~U_{F3}~G_{M}~A_{L}~G_{L}~G_{M}
P-A_{F}~C_{M}~A_{L}A_{M}~C_{F4}C_{M4}~A_{L}G_{F}~C_{M}U_{F3}~A_{L}A_{F}~G_{M}A~C_{M4}~A_{F}~C_{L4}~U_{F3}~G_{M}~A_{L}~G_{L}~G_{M}
P-A_{F}~C_{M}~A_{L}~A_{M}C_{F4}~C_{M4}A_{L}~G_{F}C_{M}~U_{F3}A_{L}~A_{F}G_{M}~AC_{M4}~A_{F}~C_{L4}~U_{F3}~G_{M}~A_{L}~G_{L}~G_{M}
P-A_{F}~C_{M}~A_{L}A_{M}~C_{F4}C_{M4}~A_{L}G_{F}~C_{M4}U_{F3}~A_{L}A_{F}~G_{M}A~C_{M4}~A_{F}~C_{L4}~U_{F3}~G_{M}~A_{L}~G_{L}~G_{M}
P-A_{F}~C_{M}~A_{L}~A_{M}C_{F4}~C_{M4}A_{L}~G_{F}C_{M4}~U_{F3}A_{L}~A_{F}G_{M}~AC_{M4}~A_{F}~C_{L4}~U_{F3}~G_{M}~A_{L}~G_{L}~G_{M}
P-A_{F}~C_{M}~A_{L2}A_{M}~C_{F4}C_{M4}~A_{L}G_{F}~C_{M}U_{F}~A_{L}A_{F}~G_{M}A~C_{M4}~A_{F}~C_{L4}~U_{F}~G_{M}~A_{L}~G_{L}~G_{M}
P-A_{F}~C_{M}~A_{L}~A_{M2}C_{F4}~C_{M4}A_{L}~G_{F}C_{M}~U_{F}A_{L}~A_{F}G_{M}~AC_{M4}~A_{F}~C_{L4}~U_{F}~G_{M}~A_{L}~G_{L}~G_{M}
P-A_{F}~C_{M}~A_{L2}A_{M}~C_{F4}C_{M4}~A_{L}G_{F}~C_{M}U_{F}~A_{L}A_{F}~G_{M}A~C_{M4}~A_{F}~C_{L4}~U_{F3}~G_{M}~A_{L}~G_{L}~G_{M}
P-A_{F}~C_{M}~A_{F}~A_{M2}C_{F4}~C_{M4}A_{L}~G_{F}C_{M}~U_{F}A_{L}~A_{F}G_{M}~AC_{M4}~A_{F}~C_{L4}~U_{F3}~G_{M}~A_{L}~G_{L}~G_{M}
P-A_{F}~C_{M}~A_{L2}A_{M}~C_{F4}C_{M4}~A_{L}G_{F}~C_{M4}U_{F}~A_{L}A_{F}~G_{M}A~C_{M4}~A_{F}~C_{L4}~U_{F3}~G_{M}~A_{L}~G_{L}~G_{M}
P-A_{F}~C_{M}~A_{L}~A_{M2}C_{F4}~C_{M4}A_{L}~G_{F}C_{M4}~U_{F}A_{L}~A_{F}G_{M}~AC_{M4}~A_{F}~C_{L4}~U_{F3}~G_{M}~A_{L}~G_{L}~G_{M}
P-A_{F}~C_{M}~A_{L2}A_{M}~C_{F4}C_{M4}~A_{L}G_{F}~C_{M}U_{F3}~A_{L}A_{F}~G_{M}A~C_{M4}~A_{F}~C_{L4}~U_{F3}~G_{M}~A_{L}~G_{L}~G_{M}
P-A_{F}~C_{M}~A_{L}~A_{M2}C_{F4}~C_{M4}A_{L}~G_{F}C_{M}~U_{F3}A_{L}~A_{F}G_{M}~AC_{M4}~A_{F}~C_{L4}~U_{F3}~G_{M}~A_{L}~G_{L}~G_{M}
P-A_{F}~C_{M}~A_{L2}A_{M}~C_{F4}C_{M4}~A_{L}G_{F}~C_{M4}U_{F3}~A_{L}A_{F}~G_{M}A~C_{M4}~A_{F}~C_{L4}~U_{F3}~G_{M}~A_{L}~G_{L}~G_{M}
P-A_{F}~C_{M}~A_{L}~A_{M2}C_{F4}~C_{M4}A_{L}~G_{F}C_{M4}~U_{F3}A_{L}~A_{F}G_{M}~AC_{M4}~A_{F}~C_{L4}~U_{F3}~G_{M}~A_{L}~G_{L}~G_{M}
P-A_{F}~C_{M}~A_{F}A_{L}~C_{F}C_{M}~A_{F}G_{L}~C_{M}U_{F}~A_{M}A_{L}~G_{M}A~C_{M}~A_{F}~C_{L}~U_{F}~G_{M}~A_{F}~G_{M}
P-A_{F}~C_{M}~A_{F}A_{L}~C_{F4}C_{M4}~A_{F}G_{L}~C_{M}U_{F}~A_{M}A_{L}~G_{M}A~C_{M4}~A_{F}~C_{L}~U_{F}~G_{M}~A_{F}~G_{M}
P-A_{F}~C_{M}~A_{F}~A_{L}C_{F4}~C_{M4}A_{F}~G_{L}C_{M}~U_{F}A_{M}~A_{L}G_{M}~AC_{M4}~A_{F}~C_{L}~U_{F}~G_{M}~A_{F}~G_{M}
P-A_{F}~C_{M}~A_{F}A_{L}~C_{F4}C_{M4}~A_{F}G_{L}~C_{M}U_{F}~A_{M}A_{L}~G_{M}A~C_{M4}~A_{F}~C_{L}~U_{F3}~G_{M}~A_{F}~G_{M}
P-A_{F}~C_{M}~A_{F}~A_{L}C_{F4}~C_{M4}A_{F}~G_{L}C_{M}~U_{F}A_{M}~A_{L}G_{M}~AC_{M4}~A_{F}~C_{L}~U_{F3}~G_{M}~A_{F}~G_{M}
P-A_{F}~C_{M}~A_{F}A_{L}~C_{F4}C_{M4}~A_{F}G_{L}~C_{M4}U_{F}~A_{M}A_{L}~G_{M}AC_{M4}~A_{F}~C_{L}~U_{F3}~G_{M}~A_{F}~G_{M}
P-A_{F}~C_{M}~A_{F}~A_{L}C_{F4}~C_{M4}A_{F}~G_{L}C_{M4}~U_{F}A_{M}~A_{L}G_{M}~AC_{M4}~A_{F}~C_{L}~U_{F3}~G_{M}~A_{F}~G_{M}
P-A_{F}~C_{M}~A_{F}A_{L}~C_{F4}C_{M4}~A_{F}G_{L}~C_{M}U_{F3}~A_{M}A_{L}~G_{M}A~C_{M4}~A_{F}~C_{L}~U_{F3}~G_{M}~A_{F}~G_{M}
P-A_{F}~C_{M}~A_{F}~A_{L}C_{F4}~C_{M4}A_{F}~G_{L}C_{M}~U_{F3}A_{M}~A_{L}G_{M}~AC_{M4}~A_{F}~C_{L}~U_{F3}~G_{M}~A_{F}~G_{M}
P-A_{F}~C_{M}~A_{F}A_{L}~C_{F4}C_{M4}~A_{F}G_{L}~C_{M4}U_{F3}~A_{M}A_{L}~G_{M}A~C_{M4}~A_{F}~C_{L}~U_{F3}~G_{M}~A_{F}~G_{M}
P-A_{F}~C_{M}~A_{F}~A_{L}C_{F4}~C_{M4}A_{F}~G_{L}C_{M4}~U_{F3}A_{M}~A_{L}G_{M}~AC_{M4}~A_{F}~C_{L}~U_{F3}~G_{M}~A_{F}~G_{M}
P-A_{F}~C_{M}~A_{F2}A_{L}~C_{F4}C_{M4}~A_{F}G_{L}~C_{M}U_{F}~A_{M}A_{L}~G_{M}A~C_{M4}~A_{F}~C_{L4}~U_{F}~G_{M}~A_{F}~G_{M}
P-A_{F}~C_{M}~A_{F}~A_{L2}C_{F4}~C_{M4}A_{F}~G_{L}C_{M}~U_{F}A_{M}~A_{L}G_{M}~AC_{M4}~A_{F}~C_{L4}~U_{F}~G_{M}~A_{F}~G_{M}
P-A_{F}~C_{M}~A_{F2}A_{L}~C_{F4}C_{M4}~A_{F}G_{L}~C_{M}U_{F}~A_{M}A_{L}~G_{M}A~C_{M4}~A_{F}~C_{L4}~U_{F3}~G_{M}~A_{F}~G_{M}
P-A_{F}~C_{M}~A_{F}~A_{L2}C_{F4}~C_{M4}A_{F}~G_{L}C_{M}~U_{F}A_{M}~A_{L}G_{M}~AC_{M4}~A_{F}~C_{L4}~U_{F3}~G_{M}~A_{F}~G_{M}
P-A_{F}~C_{M}~A_{F2}A_{L}~C_{F4}C_{M4}~A_{F}G_{L}~C_{M4}U_{F}~A_{M}A_{L}~G_{M}A~C_{M4}~A_{F}~C_{L4}~U_{F3}~G_{M}~A_{F}~G_{M}
P-A_{F}~C_{M}~A_{F}~A_{L2}C_{F4}~C_{M4}A_{F}~G_{L}C_{M4}~U_{F}A_{M}~A_{L}G_{M}~AC_{M4}~A_{F}~C_{L4}~U_{F3}~G_{M}~A_{F}~G_{M}
P-A_{F}~C_{M}~A_{F2}A_{L}~C_{F4}C_{M4}~A_{F}G_{L}~C_{M}U_{F3}~A_{M}A_{L}~G_{M}A~C_{M4}~A_{F}~C_{L4}~U_{F3}~G_{M}~A_{F}~G_{M}
P-A_{F}~C_{M}~A_{F}~A_{L2}C_{F4}~C_{M4}A_{F}~G_{L}C_{M}~U_{F3}A_{M}~A_{L}G_{M}~AC_{M4}~A_{F}~C_{L4}~U_{F3}~G_{M}~A_{F}~G_{M}
P-A_{F}~C_{M}~A_{F2}A_{L}~C_{F4}C_{M4}~A_{F}G_{L}~C_{M4}U_{F3}~A_{M}A_{L}~G_{M}A~C_{M4}~A_{F}~C_{L4}~U_{F3}~G_{M}~A_{F}~G_{M}
P-A_{F}~C_{M}~A_{F}~A_{L2}C_{F4}~C_{M4}A_{F}~G_{L}C_{M4}~U_{F3}A_{M}~A_{L}G_{M}~AC_{M4}~A_{F}~C_{L4}~U_{F3}~G_{M}~A_{F}~G_{M}
P-A_{F}~C_{M}~A_{F}A_{L}~C_{F}C_{M}~A_{F}G_{L}~C_{M}U_{F}~A_{M}A_{L}~G_{M}A~C_{M}~A_{F}~C_{L}~U_{F}~G_{M}~A_{L}~G_{L}~G_{M}
P-A_{F}~C_{M}~A_{F}A_{L}~C_{F4}C_{M4}~A_{F}G_{L}~C_{M}U_{F}~A_{M}A_{L}~G_{M}A~C_{M4}~A_{F}~C_{L}~U_{F}~G_{M}~A_{L}~G_{L}~G_{M}
P-A_{F}~C_{M}~A_{F}~A_{L}C_{F4}~C_{M4}A_{F}~G_{L}C_{M}~U_{F}A_{M}~A_{L}G_{M}~AC_{M4}~A_{F}~C_{L}~U_{F}~G_{M}~A_{L}~G_{L}~G_{M}
P-A_{F}~C_{M}~A_{F}A_{L}~C_{F4}C_{M4}~A_{F}G_{L}~C_{M}U_{F}~A_{M}A_{L}~G_{M}A~C_{M4}~A_{F}~C_{L}~U_{F3}~G_{M}~A_{L}~G_{L}~G_{M}
P-A_{F}~C_{M}~A_{F}~A_{L}C_{F4}~C_{M4}A_{F}~G_{L}C_{M}~U_{F}A_{M}~A_{L}G_{M}~AC_{M4}~A_{F}~C_{L}~U_{F3}~G_{M}~A_{L}~G_{L}~G_{M}
P-A_{F}~C_{M}~A_{F}A_{L}~C_{F4}C_{M4}~A_{F}G_{L}~C_{M4}U_{F}~A_{M}A_{L}~G_{M}A~C_{M4}~A_{F}~C_{L}~U_{F3}~G_{M}~A_{L}~G_{L}~G_{M}
P-A_{F}~C_{M}~A_{F}~A_{L}C_{F4}~C_{M4}A_{F}~G_{L}C_{M4}~U_{F}A_{M}~A_{L}G_{M}~AC_{M4}~A_{F}~C_{L}~U_{F3}~G_{M}~A_{L}~G_{L}~G_{M}
P-A_{F}~C_{M}~A_{F}A_{L}~C_{F4}C_{M4}~A_{F}G_{L}~C_{M}U_{F3}~A_{M}A_{L}~G_{M}A~C_{M4}~A_{F}~C_{L}~U_{F3}~G_{M}~A_{L}~G_{L}~G_{M}
P-A_{F}~C_{M}~A_{F}~A_{L}C_{F4}~C_{M4}A_{F}~G_{L}C_{M}~U_{F3}A_{M}~A_{L}G_{M}~AC_{M4}~A_{F}~C_{L}~U_{F3}~G_{M}~A_{L}~G_{L}~G_{M}
P-A_{F}~C_{M}~A_{F}A_{L}~C_{F4}C_{M4}~A_{F}G_{L}~C_{M4}U_{F3}~A_{M}A_{L}~G_{M}A~C_{M4}~A_{F}~C_{L}~U_{F3}~G_{M}~A_{L}~G_{L}~G_{M}
P-A_{F}~C_{M}~A_{F}~A_{L}C_{F4}~C_{M4}A_{F}~G_{L}C_{M4}~U_{F3}A_{M}~A_{L}G_{M}~AC_{M4}~A_{F}~C_{L}~U_{F3}~G_{M}~A_{L}~G_{L}~G_{M}
P-A_{F}~C_{M}~A_{F2}A_{L}~C_{F4}C_{M4}~A_{F}G_{L}~C_{M}U_{F}~A_{M}A_{L}~G_{M}A~C_{M4}~A_{F}~C_{L4}~U_{F}~G_{M}~A_{L}~G_{L}~G_{M}
P-A_{F}~C_{M}~A_{F}~A_{L2}C_{F4}~C_{M4}A_{F}~G_{L}C_{M}~U_{F}A_{M}~A_{L}G_{M}~AC_{M4}~A_{F}~C_{L4}~U_{F}~G_{M}~A_{L}~G_{L}~G_{M}
P-A_{F}~C_{M}~A_{F2}A_{L}~C_{F4}C_{M4}~A_{F}G_{L}~C_{M}U_{F}~A_{M}A_{L}~G_{M}A~C_{M4}~A_{F}~C_{L4}~U_{F3}~G_{M}~A_{L}~G_{L}~G_{M}
P-A_{F}~C_{M}~A_{F}~A_{L2}C_{F4}~C_{M4}A_{F}~G_{L}C_{M}~U_{F}A_{M}~A_{L}G_{M}~AC_{M4}~A_{F}~C_{L4}~U_{F3}~G_{M}~A_{L}~G_{L}~G_{M}
P-A_{F}~C_{M}~A_{F2}A_{L}~C_{F4}C_{M4}~A_{F}G_{L}~C_{M4}U_{F}~A_{M}A_{L}~G_{M}A~C_{M4}~A_{F}~C_{L4}~U_{F3}~G_{M}~A_{L}~G_{L}~G_{M}
P-A_{F}~C_{M}~A_{F}~A_{L2}C_{F4}~C_{M4}A_{F}~G_{L}C_{M4}~U_{F}A_{M}~A_{L}G_{M}~AC_{M4}~A_{F}~C_{L4}~U_{F3}~G_{M}~A_{L}~G_{L}~G_{M}
P-A_{F}~C_{M}~A_{F2}A_{L}~C_{F4}C_{M4}~A_{F}G_{L}~C_{M}U_{F3}~A_{M}A_{L}~G_{M}A~C_{M4}~A_{F}~C_{L4}~U_{F3}~G_{M}~A_{L}~G_{L}~G_{M}
P-A_{F}~C_{M}~A_{F}~A_{L2}C_{F4}~C_{M4}A_{F}~G_{L}C_{M}~U_{F3}A_{M}~A_{L}G_{M}~AC_{M4}~A_{F}~C_{L4}~U_{F3}~G_{M}~A_{L}~G_{L}~G_{M}
P-A_{F}~C_{M}~A_{F2}A_{L}~C_{F4}C_{M4}~A_{F}G_{L}~C_{M4}U_{F3}~A_{M}A_{L}~G_{M}A~C_{M4}~A_{F}~C_{L4}~U_{F3}~G_{M}~A_{L}~G_{L}~G_{M}
P-A_{F}~C_{M}~A_{F}~A_{L2}C_{F4}~C_{M4}A_{F}~G_{L}C_{M4}~U_{F3}A_{M}~A_{L}G_{M}~AC_{M4}~A_{F}~C_{L4}~U_{F3}~G_{M}~A_{L}~G_{L}~G_{M}
P-A_{F}~C_{M}~A_{F}A_{L}~C_{F}C_{M}~A_{L}G_{F}~C_{L}U_{F}~A_{M}A_{L}~G_{M}A~C_{L}~A_{F}~C_{M}~U_{L}~G_{M}~A_{F}~G_{M}
P-A_{F}~C_{M}~A_{F}A_{L}~C_{F4}C_{M4}~A_{L}G_{F}~C_{L}U_{F}~A_{M}A_{L}~G_{M}A~C_{L}~A_{F}~C_{M4}~U_{L}~G_{M}~A_{F}~G_{M}
P-A_{F}~C_{M}~A_{F}~A_{L}C_{F4}~C_{M4}A_{L}~G_{F}C_{L}~U_{F}A_{M}~A_{L}G_{M}~AC_{L}~A_{F}~C_{M4}~U_{L}~G_{M}~A_{F}~G_{M}
P-A_{F}~C_{M}~A_{F}A_{L}~C_{F4}C_{M4}~A_{L}G_{F}~C_{L}U_{F}~A_{M}A_{L}~G_{M}A~C_{L}~A_{F}~C_{M4}~U_{L}~G_{M}~A_{F}~G_{M}
P-A_{F}~C_{M}~A_{F}~A_{L}C_{F4}~C_{M4}A_{L}~G_{F}C_{L}~U_{F}A_{M}~A_{L}G_{M}~AC_{L}~A_{F}~C_{M4}~U_{L}~G_{M}~A_{F}~G_{M}
P-A_{F}~C_{M}~A_{F}A_{L}~C_{F4}C_{M4}~A_{L}G_{F}~C_{L}U_{F}~A_{M}A_{L}~G_{M}A~C_{L}~A_{F}~C_{M4}~U_{L}~G_{M}~A_{F}~G_{M}
P-A_{F}~C_{M}~A_{F}~A_{L}C_{F4}~C_{M4}A_{L}~G_{F}C_{L}~U_{F}A_{M}~A_{L}G_{M}~AC_{L}~A_{F}~C_{M4}~U_{L}~G_{M}~A_{F}~G_{M}
P-A_{F}~C_{M}~A_{F}A_{L}~C_{F4}C_{M4}~A_{L}G_{F}~C_{L}U_{F3}~A_{M}A_{L}~G_{M}A~C_{L}~A_{F}~C_{M4}~U_{L}~G_{M}~A_{F}~G_{M}
P-A_{F}~C_{M}~A_{F}~A_{L}C_{F4}~C_{M4}A_{L}~G_{F}C_{L}~U_{F3}A_{M}~A_{L}G_{M}~AC_{L}~A_{F}~C_{M4}~U_{L}~G_{M}~A_{F}~G_{M}
P-A_{F}~C_{M}~A_{F}A_{L}~C_{F4}C_{M4}~A_{L}G_{F}~C_{L}U_{F3}~A_{M}A_{L}~G_{M}A~C_{L}~A_{F}~C_{M4}~U_{L}~G_{M}~A_{F}~G_{M}
P-A_{F}~C_{M}~A_{F}~A_{L}C_{F4}~C_{M4}A_{L}~G_{F}C_{L}~U_{F3}A_{M}~A_{L}G_{M}~AC_{L}~A_{F}~C_{M4}~U_{L}~G_{M}~A_{F}~G_{M}
P-A_{F}~C_{M}~A_{F2}A_{L}~C_{F4}C_{M4}~A_{L}G_{F}~C_{L}U_{F}~A_{M}A_{L}~G_{M}A~C_{L4}~A_{F}~C_{M4}~U_{L}~G_{M}~A_{F}~G_{M}
P-A_{F}~C_{M}~A_{F}~A_{L2}C_{F4}~C_{M4}A_{L}~G_{F}C_{L}~U_{F}A_{M}~A_{L}G_{M}~AC_{L4}~A_{F}~C_{M4}~U_{L}~G_{M}~A_{F}~G_{M}
P-A_{F}~C_{M}~A_{F2}A_{L}~C_{F4}C_{M4}~A_{L}G_{F}~C_{L}U_{F}~A_{M}A_{L}~G_{M}A~C_{L4}~A_{F}~C_{M4}~U_{L3}~G_{M}~A_{F}~G_{M}
P-A_{F}~C_{M}~A_{F}~A_{L2}C_{F4}~C_{M4}A_{L}~G_{F}C_{L}~U_{F}A_{M}~A_{L}G_{M}~AC_{L4}~A_{F}~C_{M4}~U_{L3}~G_{M}~A_{F}~G_{M}
P-A_{F}~C_{M}~A_{F2}A_{L}~C_{F4}C_{M4}~A_{L}G_{F}~C_{L4}U_{F}~A_{M}A_{L}~G_{M}A~C_{L4}~A_{F}~C_{M4}~U_{L3}~G_{M}~A_{F}~G_{M}
P-A_{F}~C_{M}~A_{F}~A_{L2}C_{F4}~C_{M4}A_{L}~G_{F}C_{L4}~U_{F}A_{M}~A_{L}G_{M}~AC_{L4}~A_{F}~C_{M4}~U_{L3}~G_{M}~A_{F}~G_{M}
P-A_{F}~C_{M}~A_{F2}A_{L}~C_{F4}C_{M4}~A_{L}G_{F}~C_{L}U_{F3}~A_{M}A_{L}~G_{M}A~C_{L4}~A_{F}~C_{M4}~U_{L3}~G_{M}~A_{F}~G_{M}
P-A_{F}~C_{M}~A_{F}~A_{L2}C_{F4}~C_{M4}A_{L}~G_{F}C_{L}~U_{F3}A_{M}~A_{L}G_{M}~AC_{L4}~A_{F}~C_{M4}~U_{L3}~G_{M}~A_{F}~G_{M}
P-A_{F}~C_{M}~A_{F2}A_{L}~C_{F4}C_{M4}~A_{L}G_{F}~C_{L4}U_{F3}~A_{M}A_{L}~G_{M}A~C_{L4}~A_{F}~C_{M4}~U_{L3}~G_{M}~A_{F}~G_{M}
P-A_{F}~C_{M}~A_{F}~A_{L2}C_{F4}~C_{M4}A_{L}~G_{F}C_{L4}~U_{F3}A_{M}~A_{L}G_{M}~AC_{L4}~A_{F}~C_{M4}~U_{L3}~G_{M}~A_{F}~G_{M}
P-A_{F}~C_{M}~A_{F}A_{L}~C_{F}C_{M}~A_{L}G_{F}~C_{L}U_{F}~A_{M}A_{L}~G_{M}A~C_{L}~A_{F}~C_{M}~U_{L}~G_{M}~A_{L}~G_{L}~G_{M}
P-A_{F}~C_{M}~A_{F}A_{L}~C_{F4}C_{M4}~A_{L}G_{F}~C_{L}U_{F}~A_{M}A_{L}~G_{M}A~C_{L}~A_{F}~C_{M4}~U_{L}~G_{M}~A_{L}~G_{L}~G_{M}
P-A_{F}~C_{M}~A_{F}~A_{L}C_{F4}~C_{M4}A_{L}~G_{F}C_{L}~U_{F}A_{M}~A_{L}G_{M}~AC_{L}~A_{F}~C_{M4}~U_{L}~G_{M}~A_{L}~G_{L}~G_{M}
P-A_{F}~C_{M}~A_{F}A_{L}~C_{F4}C_{M4}~A_{L}G_{F}~C_{L}U_{F}~A_{M}A_{L}~G_{M}A~C_{L}~A_{F}~C_{M4}~U_{L}~G_{M}~A_{L}~G_{L}~G_{M}
P-A_{F}~C_{M}~A_{F}~A_{L}C_{F4}~C_{M4}A_{L}~G_{F}C_{L}~U_{F}A_{M}~A_{L}G_{M}~AC_{L}~A_{F}~C_{M4}~U_{L}~G_{M}~A_{L}~G_{L}~G_{M}
P-A_{F}~C_{M}~A_{F}A_{L}~C_{F4}C_{M4}~A_{L}G_{F}~C_{L}U_{F}~A_{M}A_{L}~G_{M}A~C_{L}~A_{F}~C_{M4}~U_{L}~G_{M}~A_{L}~G_{L}~G_{M}
P-A_{F}~C_{M}~A_{F}~A_{L}C_{F4}~C_{M4}A_{L}~G_{F}C_{L}~U_{F}A_{M}~A_{L}G_{M}~AC_{L}~A_{F}~C_{M4}~U_{L}~G_{M}~A_{L}~G_{L}~G_{M}
P-A_{F}~C_{M}~A_{F}A_{L}~C_{F4}C_{M4}~A_{L}G_{F}~C_{L}U_{F3}~A_{M}A_{L}~G_{M}A~C_{L}~A_{F}~C_{M4}~U_{L}~G_{M}~A_{L}~G_{L}~G_{M}
P-A_{F}~C_{M}~A_{F}~A_{L}C_{F4}~C_{M4}A_{L}~G_{F}C_{L}~U_{F3}A_{M}~A_{L}G_{M}~AC_{L}~A_{F}~C_{M4}~U_{L}~G_{M}~A_{L}~G_{L}~G_{M}
P-A_{F}~C_{M}~A_{F}A_{L}~C_{F4}C_{M4}~A_{L}G_{F}~C_{L}U_{F3}~A_{M}A_{L}~G_{M}A~C_{L}~A_{F}~C_{M4}~U_{L}~G_{M}~A_{L}~G_{L}~G_{M}
P-A_{F}~C_{M}~A_{F}~A_{L}C_{F4}~C_{M4}A_{L}~G_{F}C_{L}~U_{F3}A_{M}~A_{L}G_{M}~AC_{L}~A_{F}~C_{M4}~U_{L}~G_{M}~A_{L}~G_{L}~G_{M}
P-A_{F}~C_{M}~A_{F2}A_{L}~C_{F4}C_{M4}~A_{L}G_{F}~C_{L}U_{F}~A_{M}A_{L}~G_{M}A~C_{L4}~A_{F}~C_{M4}~U_{L}~G_{M}~A_{L}~G_{L}~G_{M}
P-A_{F}~C_{M}~A_{F}~A_{L2}C_{F4}~C_{M4}A_{L}~G_{F}C_{L}~U_{F}A_{M}~A_{L}G_{M}~AC_{L4}~A_{F}~C_{M4}~U_{L}~G_{M}~A_{L}~G_{L}~G_{M}
P-Aᵣ~C_{M}~A_{F2}A_{L}~C_{F4}C_{M4}~A_{L}G_{F}~C_{L}U_{F}~A_{M}A_{L}~G_{M}A~C_{L4}~A_{F}~C_{M4}~U_{L3}~G_{M}~A_{L}~G_{L}~G_{M}
P-A_{F}~C_{M}~A_{F}~A_{L2}C_{F4}~C_{M4}A_{L}~G_{F}C_{L}~U_{F}A_{M}~A_{L}G_{M}~AC_{L4}~A_{F}~C_{M4}~U_{L3}~G_{M}~A_{L}~G_{L}~G_{M}
P-A_{F}~C_{M}~A_{F2}A_{L}~C_{F4}C_{M4}~A_{L}G_{F}~C_{L4}U_{F}~A_{M}A_{L}~G_{M}A~C_{L4}~A_{F}~C_{M4}~U_{L3}~G_{M}~A_{L}~G_{L}~G_{M}
P-A_{F}~C_{M}~A_{F}~A_{L2}C_{F4}~C_{M4}A_{L}~G_{F}C_{L4}~U_{F}A_{M}~A_{L}G_{M}~AC_{L4}~A_{F}~C_{M4}~U_{L3}~G_{M}~A_{L}~G_{L}~G_{M}
P-A_{F}~C_{M}~A_{F2}A_{L}~C_{F4}C_{M4}~A_{L}G_{F}~C_{L}U_{F3}~A_{M}A_{L}~G_{M}A~C_{L4}~A_{F}~C_{M4}~U_{L3}~G_{M}~A_{L}~G_{L}~G_{M}
P-A_{F}~C_{M}~A_{F}~A_{L2}C_{F4}~C_{M4}A_{L}~G_{F}C_{L}~U_{F3}A_{M}~A_{L}G_{M}~AC_{L4}~A_{F}~C_{M4}~U_{L3}~G_{M}~A_{L}~G_{L}~G_{M}
P-A_{F}~C_{M}~A_{F2}A_{L}~C_{F4}C_{M4}~A_{L}G_{F}~C_{L4}U_{F3}~A_{M}A_{L}~G_{M}A~C_{L4}~A_{F}~C_{M4}~U_{L3}~G_{M}~A_{L}~G_{L}~G_{M}
P-A_{F}~C_{M}~A_{F}~A_{L2}C_{F4}~C_{M4}A_{L}~G_{F}C_{L4}~U_{F3}A_{M}~A_{L}G_{M}~AC_{L4}~A_{F}~C_{M4}~U_{L3}~G_{M}~A_{L}~G_{L}~G_{M}
P-A_{F}~C_{M}~A_{L}A_{M}~C_{F}C_{L}~A_{F}G_{F}~C_{M}U_{F}~A_{L}A_{F}~G_{L}A~C_{L}~A_{F}~C_{L}~U_{F}~G_{M}~A_{F}~G_{M}
P-A_{F}~C_{M}~A_{L}A_{M}~C_{F4}C_{L4}~A_{F}G_{F}~C_{M}U_{F}~A_{L}A_{F}~G_{L}A~C_{L4}~A_{F}~C_{L4}~U_{F}~G_{M}~A_{F}~G_{M}
P-A_{F}~C_{M}~A_{L}~A_{M}C_{F4}~C_{L4}A_{F}~G_{F}C_{M}~U_{F}A_{L}~A_{F}G_{L}~AC_{L4}~A_{F}~C_{L4}~U_{F}~G_{M}~A_{F}~G_{M}
P-A_{F}~C_{M}~A_{L}A_{M}~C_{F4}C_{L4}~A_{F}G_{F}~C_{M}U_{F}~A_{L}A_{F}~G_{L}A~C_{L4}~A_{F}~C_{L4}~U_{F3}~G_{M}~A_{F}~G_{M}
P-A_{F}~C_{M}~A_{L}~A_{M}C_{F4}~C_{L4}A_{F}~G_{F}C_{M}~U_{F}A_{L}~A_{F}G_{L}~AC_{L4}~A_{F}~C_{L4}~U_{F3}~G_{M}~A_{F}~G_{M}
P-A_{F}~C_{M}~A_{L}A_{M}~C_{F4}C_{L4}~A_{F}G_{F}~C_{M4}U_{F}~A_{L}A_{F}~G_{L}A~C_{L4}~A_{F}~C_{L4}~U_{F3}~G_{M}~A_{F}~G_{M}
P-A_{F}~C_{M}~A_{L}~A_{M}C_{F4}~C_{L4}A_{F}~G_{F}C_{M4}~U_{F}A_{L}~A_{F}G_{L}~AC_{L4}~A_{F}~C_{L4}~U_{F3}~G_{M}~A_{F}~G_{M}
P-A_{F}~C_{M}~A_{L}A_{M}~C_{F4}C_{L4}~A_{F}G_{F}~C_{M}U_{F3}~A_{L}A_{F}~G_{L}A~C_{L4}~A_{F}~C_{L4}~U_{F3}~G_{M}~A_{F}~G_{M}
P-A_{F}~C_{M}~A_{L}~A_{M}C_{F4}~C_{L4}A_{F}~G_{F}C_{M}~U_{F3}A_{L}~A_{F}G_{L}~AC_{L4}~A_{F}~C_{L4}~U_{F3}~G_{M}~A_{F}~G_{M}
P-A_{F}~C_{M}~A_{L}A_{M}~C_{F4}C_{L4}~A_{F}G_{F}~C_{M4}U_{F3}~A_{L}A_{F}~G_{L}A~C_{L4}~A_{F}~C_{L4}~U_{F3}~G_{M}~A_{F}~G_{M}
P-A_{F}~C_{M}~A_{L}~A_{M}C_{F4}~C_{L4}A_{F}~G_{F}C_{M4}~U_{F3}A_{L}~A_{F}G_{L}~AC_{L4}~A_{F}~C_{L4}~U_{F3}~G_{M}~A_{F}~G_{M}
P-A_{F}~C_{M}~A_{L2}A_{M}~C_{F4}C_{L4}~A_{F}G_{F}~C_{M}U_{F}~A_{L}A_{F}~G_{L}A~C_{L4}~A_{F}~C_{L4}~U_{F}~G_{M}~A_{F}~G_{M}
P-A_{F}~C_{M}~A_{L}~A_{M2}C_{F4}~C_{L4}A_{F}~G_{F}C_{M}~U_{F}A_{L}~A_{F}G_{L}~AC_{L4}~A_{F}~C_{L4}~U_{F}~G_{M}~A_{F}~G_{M}
P-A_{F}~C_{M}~A_{L2}A_{M}~C_{F4}C_{L4}~A_{F}G_{F}~C_{M}U_{F}~A_{L}A_{F}~G_{L}A~C_{L4}~A_{F}~C_{L4}~U_{F3}~G_{M}~A_{F}~G_{M}
P-A_{F}~C_{M}~A_{L}~A_{M2}C_{F4}~C_{L4}A_{F}~G_{F}C_{M}~U_{F}A_{L}~A_{F}G_{L}~AC_{L4}~A_{F}~C_{L4}~U_{F3}~G_{M}~A_{F}~G_{M}
P-A_{F}~C_{M}~A_{L2}A_{M}~C_{F4}C_{L4}~A_{F}G_{F}~C_{M4}U_{F}~A_{L}A_{F}~G_{L}A~C_{L4}~A_{F}~C_{L4}~U_{F3}~G_{M}~A_{F}~G_{M}
P-A_{F}~C_{M}~A_{L}~A_{M2}C_{F4}~C_{L4}A_{F}~G_{F}C_{M4}~U_{F}A_{L}~A_{F}G_{L}~AC_{L4}~A_{F}~C_{L4}~U_{F3}~G_{M}~A_{F}~G_{M}
P-A_{F}~C_{M}~A_{L2}A_{M}~C_{F4}C_{L4}~A_{F}G_{F}~C_{M}U_{F3}~A_{L}A_{F}~G_{L}A~C_{L4}~A_{F}~C_{L4}~U_{F3}~G_{M}~A_{F}~G_{M}
P-A_{F}~C_{M}~A_{L}~A_{M2}C_{F4}~C_{L4}A_{F}~G_{F}C_{M}~U_{F3}A_{L}~A_{F}G_{L}~AC_{L4}~A_{F}~C_{L4}~U_{F3}~G_{M}~A_{F}~G_{M}
P-A_{F}~C_{M}~A_{L2}A_{M}~C_{F4}C_{L4}~A_{F}G_{F}~C_{M4}U_{F3}~A_{L}A_{F}~G_{L}A~C_{L4}~A_{F}~C_{L4}~U_{F3}~G_{M}~A_{F}~G_{M}
P-A_{F}~C_{M}~A_{L}~A_{M2}C_{F4}~C_{L4}A_{F}~G_{F}C_{M4}~U_{F3}A_{L}~A_{F}G_{L}~AC_{L4}~A_{F}~C_{L4}~U_{F3}~G_{M}~A_{F}~G_{M}
P-A_{F}~C_{M}~A_{L}A_{M}~C_{F}C_{L}~A_{F}G_{F}~C_{M}U_{F}~A_{L}A_{F}~G_{L}A~C_{L}~A_{F}~C_{L}~U_{F}~G_{M}~A_{L}~G_{L}~G_{M}
P-A_{F}~C_{M}~A_{L}A_{M}~C_{F4}C_{L4}~A_{F}G_{F}~C_{M}U_{F}~A_{L}A_{F}~G_{L}A~C_{L4}~A_{F}~C_{L4}~U_{F}~G_{M}~A_{L}~G_{L}~G_{M}
P-A_{F}~C_{M}~A_{L}~A_{M}C_{F4}~C_{L4}A_{F}~G_{F}C_{M}~U_{F}A_{L}~A_{F}G_{L}~AC_{L4}~A_{F}~C_{L4}~U_{F}~G_{M}~A_{L}~G_{L}~G_{M}
P-A_{F}~C_{M}~A_{L}A_{M}~C_{F4}C_{L4}~A_{F}G_{F}~C_{M}U_{F}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M}C_{F4}~C_{L4}A_{F}~G_{F}C_{M}~U_{F}A_{L}~A_{F}G_{L}~AC_{L4}~A_{F}~C_{L4}~U_{F3}~G_{M}~A_{L}~G_{L}~G_{M}
P-A_{F}~C_{M}~A_{L}A_{M}~C_{F4}C_{L4}~A_{F}G_{F}~C_{M4}U_{F}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M}C_{F4}~C_{L4}A_{F}~G_{F}C_{M4}~U_{F}A_{L}~A_{F}G_{L}~AC_{L4}~A_{F}~C_{L4}~U_{F3}~G_{M}~A_{L}~G_{L}~G_{M}
P-A_{F}~C_{M}~A_{L}A_{M}~C_{F4}C_{L4}~A_{F}G_{F}~C_{M}U_{F3}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M}C_{F4}~C_{L4}A_{F}~G_{F}C_{M}~U_{F3}A_{L}~A_{F}G_{L}~AC_{L4}~A_{F}~C_{L4}~U_{F3}~G_{M}~A_{L}~G_{L}~G_{M}
P-A_{F}~C_{M}~A_{L}A_{M}~C_{F4}C_{L4}~A_{F}G_{F}~C_{M4}U_{F3}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M}C_{F4}~C_{L4}A_{F}~G_{F}C_{M4}~U_{F3}A_{L}~A_{F}G_{L}~AC_{L4}~A_{F}~C_{L4}~U_{F3}~G_{M}~A_{L}~G_{L}~G_{M}
P-A_{F}~C_{M}~A_{L2}A_{M}~C_{F4}C_{L4}~A_{F}G_{F}~C_{M}U_{F}~A_{L}A_{F}~G_{L}A~C_{L4}~A_{F}~C_{L4}~U_{F}~G_{M}~A_{L}~G_{L}~G_{M}
P-A_{F}~C_{M}~A_{L}~A_{M2}C_{F4}~C_{L4}A_{F}~G_{F}C_{M}~U_{F}A_{L}~A_{F}G_{L}~AC_{L4}~A_{F}~C_{L4}~U_{F}~G_{M}~A_{L}~G_{L}~G_{M}
P-A_{F}~C_{M}~A_{L2}A_{M}~C_{F4}C_{L4}~A_{F}G_{F}~C_{M}U_{F}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M2}C_{F4}~C_{L4}A_{F}~G_{F}C_{M}~U_{F}A_{L}~A_{F}G_{L}~AC_{L4}~A_{F}~C_{L4}~U_{F3}~G_{M}~A_{L}~G_{L}~G_{M}
P-A_{F}~C_{M}~A_{L2}A_{M}~C_{F4}C_{L4}~A_{F}G_{F}~C_{M4}U_{F}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M2}C_{F4}~C_{L4}A_{F}~G_{F}C_{M4}~U_{F}A_{L}~A_{F}G_{L}~AC_{L4}~A_{F}~C_{L4}~U_{F3}~G_{M}~A_{L}~G_{L}~G_{M}
P-A_{F}~C_{M}~A_{L2}A_{M}~C_{F4}C_{L4}~A_{F}G_{F}~C_{M}U_{F3}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M2}C_{F4}~C_{L4}A_{F}~G_{F}C_{M}~U_{F3}A_{L}~A_{F}G_{L}~AC_{L4}~A_{F}~C_{L4}~U_{F3}~G_{M}~A_{L}~G_{L}~G_{M}
P-A_{F}~C_{M}~A_{L2}A_{M}~C_{F4}C_{L4}~A_{F}G_{F}~C_{M4}U_{F3}~A_{L}A_{F}~G_{L}A~C_{L4}~A_{F}~C_{L4}~U_{F3}~G_{M}~A_{L}~G_{L}~G_{M}
P-A_{F}~C_{M}~A_{L}~A_{M2}C_{F4}~C_{L4}A_{F}~G_{F}C_{M4}~U_{F3}A_{L}~A_{F}G_{L}~AC_{L4}~A_{F}~C_{L4}~U_{F3}~G_{M}~A_{L}~G_{L}~G_{M}

TABLE 7B
Compositions of Matter Suitable For Use in vitro or in a
Subject with a Protective Carrier (5′-3′)

P-A_{F}~C_{F}~A_{F}A_{F}~C_{F4}~C_{F4}~A_{F}G_{F}C_{F}U_{F}~A_{F}A_{F}G_{F}A~C_{F4}~A_{F}~C_{F4}U_{F}~G_{F}A_{L}~G_{L}~G
P-A_{F}~C_{F}~A_{F}A_{F}~C_{F4}~C_{F4}~A_{F}G_{F}C_{F}U_{F}~A_{F}A_{F}G_{F}A~C_{F4}~A_{F}~C_{F4}U_{F}~G_{F}A_{L}~G_{L}~G
P-A_{F}~C_{F}~A_{F}A_{F}~C_{F4}~C_{F4}~A_{F}G_{F}C_{F}U_{F}~A_{F}A_{F}G_{F}A~C_{F4}~A_{F}~C_{F4}U_{F3}~G_{F}A_{L}~G_{L}~G
P-A_{F}~C_{F}~A_{F}A_{F}~C_{F4}~C_{F4}~A_{F}G_{F}C_{F}U_{F}~A_{F}A_{F}G_{F}A~C_{F4}~A_{F}~C_{F4}U_{F3}~G_{F}A_{L}~G_{L}~G
P-A_{F}~C_{F}~A_{F}A_{F}~C_{F4}~C_{F4}~A_{F}G_{F}C_{F4}U_{F}~A_{F}A_{F}G_{F}A~C_{F4}~A_{F}~C_{F4}U_{F3}~G_{F}A_{L}~G_{L}~G
P-A_{F}~C_{F}~A_{F}A_{F}~C_{F4}~C_{F4}~A_{F}G_{F}C_{F4}U_{F}~A_{F}A_{F}G_{F}A~C_{F4}~A_{F}~C_{F4}U_{F3}~G_{F}A_{L}~G_{L}~G
P-A_{F}~C_{F}~A_{F}A_{F}~C_{F4}~C_{F4}~A_{F}G_{F}C_{F}U_{F3}~A_{F}A_{F}G_{F}A~C_{F4}~A_{F}~C_{F4}U_{F3}~G_{F}A_{L}~G_{L}~G
P-A_{F}~C_{F}~A_{F}A_{F}~C_{F4}~C_{F4}~A_{F}G_{F}C_{F}U_{F3}~A_{F}A_{F}G_{F}A~C_{F4}~A_{F}~C_{F4}U_{F3}~G_{F}A_{L}~G_{L}~G
P-A_{F}~C_{F}~A_{F}A_{F}~C_{F4}~C_{F4}~A_{F}G_{F}C_{F4}U_{F3}~A_{F}A_{F}G_{F}A~C_{F4}~A_{F}~C_{F4}U_{F3}~G_{F}A_{L}~G_{L}~G
P-A_{F}~C_{F}~A_{F}A_{F}~C_{F4}~C_{F4}~A_{F}G_{F}C_{F4}U_{F3}~A_{F}A_{F}G_{F}A~C_{F4}~A_{F}~C_{F4}U_{F3}~G_{F}A_{L}~G_{L}~G
P-A_{F}~C_{F}~A_{F2}A_{F}~C_{F4}~C_{F4}~A_{F}G_{F}C_{F}U_{F}~A_{F}A_{F}G_{F}A~C_{F4}~A_{F}~C_{F4}U_{F}~G_{F}A_{L}~G_{L}~G
P-A_{F}~C_{F}~A_{F}A_{F2}~C_{F4}~C_{F4}~A_{F}G_{F}C_{F}U_{F}~A_{F}A_{F}G_{F}A~C_{F4}~A_{F}~C_{F4}U_{F}~G_{F}A_{L}~G_{L}~G
P-A_{F}~C_{F}~A_{F2}A_{F}~C_{F4}~C_{F4}~A_{F}G_{F}C_{F}U_{F}~A_{F}A_{F}G_{F}A~C_{F4}~A_{F}~C_{F4}U_{F3}~G_{F}A_{L}~G_{L}~G
P-A_{F}~C_{F}~A_{F}A_{F2}~C_{F4}~C_{F4}~A_{F}G_{F}C_{F}U_{F}~A_{F}A_{F}G_{F}A~C_{F4}~A_{F}~C_{F4}U_{F3}~G_{F}A_{L}~G_{L}~G
P-A_{F}~C_{F}~A_{F2}A_{F}~C_{F4}~C_{F4}~A_{F}G_{F}C_{F4}U_{F}~A_{F}A_{F}G_{F}A~C_{F4}~A_{F}~C_{F4}U_{F3}~G_{F}A_{L}~G_{L}~G
P-A_{F}~C_{F}~A_{F}A_{F2}~C_{F4}~C_{F4}~A_{F}G_{F}C_{F4}U_{F}~A_{F}A_{F}G_{F}A~C_{F4}~A_{F}~C_{F4}U_{F3}~G_{F}A_{L}~G_{L}~G
P-A_{F}~C_{F}~A_{F2}A_{F}~C_{F4}~C_{F4}~A_{F}G_{F}C_{F}U_{F3}~A_{F}A_{F}G_{F}A~C_{F4}~A_{F}~C_{F4}U_{F3}~G_{F}A_{L}~G_{L}~G
P-A_{F}~C_{F}~A_{F}A_{F2}~C_{F4}~C_{F4}~A_{F}G_{F}C_{F}U_{F3}~A_{F}A_{F}G_{F}A~C_{F4}~A_{F}~C_{F4}U_{F3}~G_{F}A_{L}~G_{L}~G
P-A_{F}~C_{F}~A_{F2}A_{F}~C_{F4}~C_{F4}~A_{F}G_{F}C_{F4}U_{F3}~A_{F}A_{F}G_{F}A~C_{F4}~A_{F}~C_{F4}U_{F3}~G_{F}A_{L}~G_{L}~G
P-A_{F}~C_{F}~A_{F}A_{F2}~C_{F4}~C_{F4}~A_{F}G_{F}C_{F4}U_{F3}~A_{F}A_{F}G_{F}A~C_{F4}~A_{F}~C_{F4}U_{F3}~G_{F}A_{L}~G_{L}~G
P-A_{F}~C_{F}~A_{L}A_{F}~C_{F}~C_{F}~A_{L}G_{F}C_{F}U_{F}~A_{F}A_{L}G_{F}A~C_{F}~A_{F}~C_{L}U_{F}~G_{F}A_{L}~G_{L}~G
P-A_{F}~C_{F}~A_{L}A_{F}~C_{F4}~C_{F4}~A_{L}G_{F}C_{F}U_{F}~A_{L}A_{F}G_{F}A~C_{F4}~A_{F}~C_{L4}U_{F}~G_{F}A_{L}~G_{L}~G
P-A_{F}~C_{F}~A_{L}A_{F}~C_{F4}~C_{F4}~A_{L}G_{F}C_{F}U_{F}~A_{L}A_{F}G_{F}A~C_{F4}~A_{F}~C_{L4}U_{F3}~G_{F}A_{L}~G_{L}~G
P-A_{F}~C_{F}~A_{L}A_{F}~C_{F4}~C_{F4}~A_{L}G_{F}C_{F}U_{F}~A_{L}A_{F}G_{F}A~C_{F4}~A_{F}~C_{L4}U_{F3}~G_{F}A_{L}~G_{L}~G
P-A_{F}~C_{F}~A_{L}A_{F}~C_{F4}~C_{F4}~A_{L}G_{F}C_{F4}U_{F}~A_{L}A_{F}G_{F}A~C_{F4}~A_{F}~C_{L4}U_{F3}~G_{F}A_{L}~G_{L}~G
P-A_{F}~C_{F}~A_{L}A_{F}~C_{F4}~C_{F4}~A_{L}G_{F}C_{F4}U_{F}~A_{L}A_{F}G_{F}A~C_{F4}~A_{F}~C_{L4}U_{F3}~G_{F}A_{L}~G_{L}~G
P-A_{F}~C_{F}~A_{L}A_{F}~C_{F4}~C_{F4}~A_{L}G_{F}C_{F}U_{F3}~A_{L}A_{F}G_{F}A~C_{F4}~A_{F}~C_{L4}U_{F3}~G_{F}A_{L}~G_{L}~G
P-A_{F}~C_{F}~A_{L}A_{F}~C_{F4}~C_{F4}~A_{L}G_{F}C_{F}U_{F3}~A_{L}A_{F}G_{F}A~C_{F4}~A_{F}~C_{L4}U_{F3}~G_{F}A_{L}~G_{L}~G
P-A_{F}~C_{F}~A_{L}A_{F}~C_{F4}~C_{F4}~A_{L}G_{F}C_{F4}U_{F3}~A_{L}A_{F}G_{F}A~C_{F4}~A_{F}~C_{L4}U_{F3}~G_{F}A_{L}~G_{L}~G
P-A_{F}~C_{F}~A_{L}A_{F}~C_{F4}~C_{F4}~A_{L}G_{F}C_{F4}U_{F3}~A_{L}A_{F}G_{F}A~C_{F4}~A_{F}~C_{L4}U_{F3}~G_{F}A_{L}~G_{L}~G
P-A_{F}~C_{F}~A_{L2}A_{F}~C_{F4}~C_{F4}~A_{L}G_{F}C_{F}U_{F}~A_{L}A_{F}G_{F}A~C_{F4}~A_{F}~C_{L4}U_{F}~G_{F}A_{L}~G_{L}~G
P-A_{F}~C_{F}~A_{L}A_{F2}~C_{F4}~C_{F4}~A_{L}G_{F}C_{F}U_{F}~A_{L}A_{F}G_{F}A~C_{F4}~A_{F}~C_{L4}U_{F}~G_{F}A_{L}~G_{L}~G
P-A_{F}~C_{F}~A_{L2}A_{F}~C_{F4}~C_{F4}~A_{L}G_{F}C_{F}U_{F}~A_{L}A_{F}G_{F}A~C_{F4}~A_{F}~C_{L4}U_{F3}~G_{F}A_{L}~G_{L}~G
P-A_{F}~C_{F}~A_{F}A_{F2}~C_{F4}~C_{F4}~A_{L}G_{F}C_{F}U_{F}~A_{L}A_{F}G_{F}A~C_{F4}~A_{F}~C_{L4}U_{F3}~G_{F}A_{L}~G_{L}~G
P-A_{F}~C_{F}~A_{L2}A_{F}~C_{F4}~C_{F4}~A_{L}G_{F}C_{F4}U_{F}~A_{L}A_{F}G_{F}A~C_{F4}~A_{F}~C_{L4}U_{F3}~G_{F}A_{L}~G_{L}~G
P-A_{F}~C_{F}~A_{L}A_{F2}~C_{F4}~C_{F4}~A_{L}G_{F}C_{F4}U_{F}~A_{L}A_{F}G_{F}A~C_{F4}~A_{F}~C_{L4}U_{F3}~G_{F}A_{L}~G_{L}~G
P-A_{F}~C_{F}~A_{L2}A_{F}~C_{F4}~C_{F4}~A_{L}G_{F}C_{F}U_{F3}~A_{L}A_{F}G_{F}A~C_{F4}~A_{F}~C_{L4}U_{F3}~G_{F}A_{L}~G_{L}~G
P-A_{F}~C_{F}~A_{L}A_{F2}~C_{F4}~C_{F4}~A_{L}G_{F}C_{F}U_{F3}~A_{L}A_{F}G_{F}A~C_{F4}~A_{F}~C_{L4}U_{F3}~G_{F}A_{L}~G_{L}~G
P-A_{F}~C_{F}~A_{L2}A_{F}~C_{F4}~C_{F4}~A_{L}G_{F}C_{F4}U_{F3}~A_{L}A_{F}G_{F}A~C_{F4}~A_{F}~C_{L4}U_{F3}~G_{F}A_{L}~G_{L}~G
P-A_{F}~C_{F}~A_{L}A_{F2}~C_{F4}~C_{F4}~A_{L}G_{F}C_{F4}U_{F3}~A_{L}A_{F}G_{F}A~C_{F4}~A_{F}~C_{L4}U_{F3}~G_{F}A_{L}~G_{L}~G
P-A_{F}~C_{F}~A_{F}A_{L}~C_{F}~C_{F}~A_{F}G_{L}C_{F}U_{F}~A_{F}A_{L}G_{F}A~C_{F}~A_{F}~C_{L}U_{F}~G_{F}A_{L}~G_{L}~G
P-A_{F}~C_{F}~A_{F}A_{L}~C_{F4}~C_{F4}~A_{F}G_{L}C_{F}U_{F}~A_{F}A_{L}G_{F}A~C_{F4}~A_{F}~C_{L}U_{F}~G_{F}A_{L}~G_{L}~G
P-A_{F}~C_{F}~A_{F}A_{L}~C_{F4}~C_{F4}~A_{F}G_{L}C_{F}U_{F}~A_{F}A_{L}G_{F}A~C_{F4}~A_{F}~C_{L}U_{F}~G_{F}A_{L}~G_{L}~G
P-A_{F}~C_{F}~A_{F}A_{L}~C_{F4}~C_{F4}~A_{F}G_{L}C_{F}U_{F}~A_{F}A_{L}G_{F}A~C_{F4}~A_{F}~C_{L}U_{F3}~G_{F}A_{L}~G_{L}~G
P-A_{F}~C_{F}~A_{F}A_{L}~C_{F4}~C_{F4}~A_{F}G_{L}C_{F}U_{F}~A_{F}A_{L}G_{F}A~C_{F4}~A_{F}~C_{L}U_{F3}~G_{F}A_{L}~G_{L}~G
P-A_{F}~C_{F}~A_{F}A_{L}~C_{F4}~C_{F4}~A_{F}G_{L}C_{F4}U_{F}~A_{F}A_{L}G_{F}A~C_{F4}~A_{F}~C_{L}U_{F3}~G_{F}A_{L}~G_{L}~G
P-A_{F}~C_{F}~A_{F}A_{L}~C_{F4}~C_{F4}~A_{F}G_{L}C_{F4}U_{F}~A_{F}A_{L}G_{F}A~C_{F4}~A_{F}~C_{L}U_{F3}~G_{F}A_{L}~G_{L}~G
P-A_{F}~C_{F}~A_{F}A_{L}~C_{F4}~C_{F4}~A_{F}G_{L}C_{F}U_{F3}~A_{F}A_{L}G_{F}A~C_{F4}~A_{F}~C_{L}U_{F3}~G_{F}A_{L}~G_{L}~G
P-A_{F}~C_{F}~A_{F}A_{L}~C_{F4}~C_{F4}~A_{F}G_{L}C_{F}U_{F3}~A_{F}A_{L}G_{F}A~C_{F4}~A_{F}~C_{L}U_{F3}~G_{F}A_{L}~G_{L}~G
P-A_{F}~C_{F}~A_{F}A_{L}~C_{F4}~C_{F4}~A_{F}G_{L}C_{F4}U_{F3}~A_{F}A_{L}G_{F}A~C_{F4}~A_{F}~C_{L}U_{F3}~G_{F}A_{L}~G_{L}~G
P-A_{F}~C_{F}~A_{F}A_{L}~C_{F4}~C_{F4}~A_{F}G_{L}C_{F4}U_{F3}~A_{F}A_{L}G_{F}A~C_{F4}~A_{F}~C_{L}U_{F3}~G_{F}A_{L}~G_{L}~G
P-A_{F}~C_{F}~A_{F2}A_{L}~C_{F4}~C_{F4}~A_{F}G_{L}C_{F}U_{F}~A_{F}A_{L}G_{F}A~C_{F4}~A_{F}~C_{L4}U_{F}~G_{F}A_{L}~G_{L}~G
P-A_{F}~C_{F}~A_{F}A_{L2}~C_{F4}~C_{F4}~A_{F}G_{L}C_{F}U_{F}~A_{F}A_{L}G_{F}A~C_{F4}~A_{F}~C_{L4}U_{F}~G_{F}A_{L}~G_{L}~G
P-A_{F}~C_{F}~A_{F2}A_{L}~C_{F4}~C_{F4}~A_{F}G_{L}C_{F}U_{F}~A_{F}A_{L}G_{F}A~C_{F4}~A_{F}~C_{L4}U_{F3}~G_{F}A_{L}~G_{L}~G
P-A_{F}~C_{F}~A_{F}A_{L2}~C_{F4}~C_{F4}~A_{F}G_{L}C_{F}U_{F}~A_{F}A_{L}G_{F}A~C_{F4}~A_{F}~C_{L4}U_{F3}~G_{F}A_{L}~G_{L}~G
P-A_{F}~C_{F}~A_{F2}A_{L}~C_{F4}~C_{F4}~A_{F}G_{L}C_{F4}U_{F}~A_{F}A_{L}G_{F}A~C_{F4}~A_{F}~C_{L4}U_{F3}~G_{F}A_{L}~G_{L}~G
P-A_{F}~C_{F}~A_{F}A_{L2}~C_{F4}~C_{F4}~A_{F}G_{L}C_{F4}U_{F}~A_{F}A_{L}G_{F}A~C_{F4}~A_{F}~C_{L4}U_{F3}~G_{F}A_{L}~G_{L}~G
P-A_{F}~C_{F}~A_{F2}A_{L}~C_{F4}~C_{F4}~A_{F}G_{L}C_{F}U_{F3}~A_{F}A_{L}G_{F}A~C_{F4}~A_{F}~C_{L4}U_{F3}~G_{F}A_{L}~G_{L}~G
P-A_{F}~C_{F}~A_{F}A_{L2}~C_{F4}~C_{F4}~A_{F}G_{L}C_{F}U_{F3}~A_{F}A_{L}G_{F}A~C_{F4}~A_{F}~C_{L4}U_{F3}~G_{F}A_{L}~G_{L}~G
P-A_{F}~C_{F}~A_{F2}A_{L}~C_{F4}~C_{F4}~A_{F}G_{L}C_{F4}U_{F3}~A_{F}A_{L}G_{F}A~C_{F4}~A_{F}~C_{L4}U_{F3}~G_{F}A_{L}~G_{L}~G
P-A_{F}~C_{F}~A_{F}A_{L2}~C_{F4}~C_{F4}~A_{F}G_{L}C_{F4}U_{F3}~A_{F}A_{L}G_{F}A~C_{F4}~A_{F}~C_{L4}U_{F3}~G_{F}A_{L}~G_{L}~G
P-A_{F}~C_{F}~A_{F}A_{L}~C_{F}~C_{F}~A_{L}G_{F}C_{L}U_{F}~A_{F}A_{L}G_{F}A~C_{L}~A_{F}~C_{F}U_{L}~G_{F}A_{L}~G_{L}~G
P-A_{F}~C_{F}~A_{F}A_{L}~C_{F4}~C_{F4}~A_{L}G_{F}C_{L}U_{F}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F3}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F3}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F3}~A_{F}A_{L}G_{F}A~C_{L}~A_{F}~C_{F4}U_{L}~G_{F}A_{L}~G_{L}~G
P-A_{F}~C_{F}~A_{F}A_{L}~C_{F4}~C_{F4}~A_{L}G_{F}C_{L}U_{F3}~A_{F}A_{L}G_{F}A~C_{L}~A_{F}~C_{F4}U_{L}~G_{F}A_{L}~G_{L}~G
P-A_{F}~C_{F}~A_{F2}A_{L}~C_{F4}~C_{F4}~A_{L}G_{F}C_{L}U_{F}~A_{F}A_{L}G_{F}A~C_{L4}~A_{F}~C_{F4}U_{L}~G_{F}A_{L}~G_{L}~G
P-A_{F}~C_{F}~A_{F}A_{L2}~C_{F4}~C_{F4}~A_{L}G_{F}C_{L}U_{F}~A_{F}A_{L}G_{F}A~C_{L4}~A_{F}~C_{F4}U_{L}~G_{F}A_{L}~G_{L}~G
P-A_{F}~C_{F}~A_{F2}A_{L}~C_{F4}~C_{F4}~A_{L}G_{F}C_{L}U_{F}~A_{F}A_{L}G_{F}A~C_{L4}~A_{F}~C_{F4}U_{L3}~G_{F}A_{L}~G_{L}~G
P-A_{F}~C_{F}~A_{F}A_{L2}~C_{F4}~C_{F4}~A_{L}G_{F}C_{L}U_{F}~A_{F}A_{L}G_{F}A~C_{L4}~A_{F}~C_{F4}U_{L3}~G_{F}A_{L}~G_{L}~G
P-A_{F}~C_{F}~A_{F2}A_{L}~C_{F4}~C_{F4}~A_{L}G_{F}C_{L4}U_{F}~A_{F}A_{L}G_{F}A~C_{L4}~A_{F}~C_{F4}U_{L3}~G_{F}A_{L}~G_{L}~G
P-A_{F}~C_{F}~A_{F}A_{L2}~C_{F4}~C_{F4}~A_{L}G_{F}C_{L4}U_{F}~A_{F}A_{L}G_{F}A~C_{L4}~A_{F}~C_{F4}U_{L3}~G_{F}A_{L}~G_{L}~G
P-A_{F}~C_{F}~A_{F2}A_{L}~C_{F4}~C_{F4}~A_{L}G_{F}C_{L}U_{F3}~A_{F}A_{L}G_{F}A~C_{L4}~A_{F}~C_{F4}U_{L3}~G_{F}A_{L}~G_{L}~G
P-A_{F}~C_{F}~A_{F}A_{L2}~C_{F4}~C_{F4}~A_{L}G_{F}C_{L}U_{F3}~A_{F}A_{L}G_{F}A~C_{L4}~A_{F}~C_{F4}U_{L3}~G_{F}A_{L}~G_{L}~G
P-A_{F}~C_{F}~A_{F2}A_{L}~C_{F4}~C_{F4}~A_{L}G_{F}C_{L4}U_{F3}~A_{F}A_{L}G_{F}A~C_{L4}~A_{F}~C_{F4}U_{L3}~G_{F}A_{L}~G_{L}~G
P-A_{F}~C_{F}~A_{F}A_{L2}~C_{F4}~C_{F4}~A_{L}G_{F}C_{L4}U_{F3}~A_{F}A_{L}G_{F}A~C_{L4}~A_{F}~C_{F4}U_{L3}~G_{F}A_{L}~G_{L}~G
P-A_{F}~C_{F}~A_{L}A_{F}~C_{F}~C_{L}~A_{F}G_{F}C_{F}U_{F}~A_{L}A_{F}G_{L}A~C_{L}~A_{F}~C_{L}U_{F-}G_{F}A_{L}~G_{L}~G
P-A_{F}~C_{F}~A_{L}A_{F}~C_{F4}~C_{L4}~A_{F}G_{F}C_{F}U_{F}~A_{L}A_{F}G_{L}A~C_{L4}~A_{F}~C_{L4}U_{F}~G_{F}A_{L}~G_{L}~G
P-A_{F}~C_{F}~A_{L}A_{F}~C_{F4}~C_{L4}~A_{F}G_{F}C_{F}U_{F}~A_{L}A_{F}G_{L}A~C_{L4}~A_{F}~C_{L4}U_{F}~G_{F}A_{L}~G_{L}~G
P-A_{F}~C_{F}~A_{L}A_{F}~C_{F4}~C_{L4}~A_{F}G_{F}C_{F}U_{F}~A_{L}A_{F}G_{L}A~C_{L4}~A_{F}~C_{L4}U_{F3}~G_{F}A_{L}~G_{L}~G
P-A_{F}~C_{F}~A_{L}A_{F}~C_{F4}~C_{L4}~A_{F}G_{F}C_{F}U_{F}~A_{L}A_{F}G_{L}A~C_{L4}~A_{F}~C_{L4}U_{F3}~G_{F}A_{L}~G_{L}~G
P-A_{F}~C_{F}~A_{L}A_{F}~C_{F4}~C_{L4}~A_{F}G_{F}C_{F4}U_{F}~A_{L}A_{F}G_{L}A~C_{L4}~A_{F}~C_{L4}U_{F3}~G_{F}A_{L}~G_{L}~G
P-A_{F}~C_{F}~A_{L}A_{F}~C_{F4}~C_{L4}~A_{F}G_{F}C_{F4}U_{F}~A_{L}A_{F}G_{L}A~C_{L4}~A_{F}~C_{L4}U_{F3}~G_{F}A_{L}~G_{L}~G
P-A_{F}~C_{F}~A_{L}A_{F}~C_{F4}~C_{L4}~A_{F}G_{F}C_{F}U_{F3}~A_{L}A_{F}G_{L}A~C_{L4}~A_{F}~C_{L4}U_{F3}~G_{F}A_{L}~G_{L}~G
P-A_{F}~C_{F}~A_{L}A_{F}~C_{F4}~C_{L4}~A_{F}G_{F}C_{F}U_{F3}~A_{L}A_{F}G_{L}A~C_{L4}~A_{F}~C_{L4}U_{F3}~G_{F}A_{L}~G_{L}~G
P-A_{F}~C_{F}~A_{L}A_{F}~C_{F4}~C_{L4}~A_{F}G_{F}C_{F4}U_{F3}~A_{L}A_{F}G_{L}A~C_{L4}~A_{F}~C_{L4}U_{F3}~G_{F}A_{L}~G_{L}~G
P-A_{F}~C_{F}~A_{L}A_{F}~C_{F4}~C_{L4}~A_{F}G_{F}C_{F4}U_{F3}~A_{L}A_{F}G_{L}A~C_{L4}~A_{F}~C_{L4}U_{F3}~G_{F}A_{L}~G_{L}~G
P-A_{F}~C_{F}~A_{L2}A_{F}~C_{F4}~C_{L4}~A_{F}G_{F}C_{F}U_{F}~A_{L}A_{F}G_{L}A~C_{L4}~A_{F}~C_{L4}U_{F}~G_{F}A_{L}~G_{L}~G
P-A_{F}~C_{F}~A_{L}A_{F2}~C_{F4}~C_{L4}~A_{F}G_{F}C_{F}U_{F}~A_{L}A_{F}G_{L}A~C_{L4}~A_{F}~C_{L4}U_{F}~G_{F}A_{L}~G_{L}~G
P-A_{F}~C_{F}~A_{L2}A_{F}~C_{F4}~C_{L4}~A_{F}G_{F}C_{F}U_{F}~A_{L}A_{F}G_{L}A~C_{L4}~A_{F}~C_{L4}U_{F3}~G_{F}A_{L}~G_{L}~G
P-A_{F}~C_{F}~A_{L}A_{F2}~C_{F4}~C_{L4}~A_{F}G_{F}C_{F}U_{F}~A_{L}A_{F}G_{L}A~C_{L4}~A_{F}~C_{L4}U_{F3}~G_{F}A_{L}~G_{L}~G
P-A_{F}~C_{F}~A_{L2}A_{F}~C_{F4}~C_{L4}~A_{F}G_{F}C_{F4}U_{F}~A_{L}A_{F}G_{L}A~C_{L4}~A_{F}~C_{L4}U_{F3}~G_{F}A_{L}~G_{L}~G
P-A_{F}~C_{F}~A_{L}A_{F2}~C_{F4}~C_{L4}~A_{F}G_{F}C_{F4}U_{F}~A_{L}A_{F}G_{L}A~C_{L4}~A_{F}~C_{L4}U_{F3}~G_{F}A_{L}~G_{L}~G
P-A_{F}~C_{F}~A_{L2}A_{F}~C_{F4}~C_{L4}~A_{F}G_{F}C_{F}U_{F3}~A_{L}A_{F}G_{L}A~C_{L4}~A_{F}~C_{L4}U_{F3}~G_{F}A_{L}~G_{L}~G
P-A_{F}~C_{F}~A_{L}A_{F2}~C_{F4}~C_{L4}~A_{F}G_{F}C_{F}U_{F3}~A_{L}A_{F}G_{L}A~C_{L4}~A_{F}~C_{L4}U_{F3}~G_{F}A_{L}~G_{L}~G
P-A_{F}~C_{F}~A_{L2}A_{F}~C_{F4}~C_{L4}~A_{F}G_{F}C_{F4}U_{F3}~A_{L}A_{F}G_{L}A~C_{L4}~A_{F}~C_{L4}U_{F3}~G_{F}A_{L}~G_{L}~G
P-A_{F}~C_{F}~A_{L}A_{F2}~C_{F4}~C_{L4}~A_{F}G_{F}C_{F4}U_{F3}~A_{L}A_{F}G_{L}A~C_{L4}~A_{F}~C_{L4}U_{F3}~G_{F}A_{L}~G_{L}~G

### Table 8

### Examples of Murine/Human PTEN ags-siRNA Compositions

### A. Murine/Human PTEN Antisense Sequence:

UUGUCUCUGGUCCUUACU

Lima et al. (Cell 150: 883-94, 2012) used this sequence to generate ssRNAi compounds against human/murine PTEN including the following:

5′-VP-Tₘₒₑ~U_{F}~G_{M}U_{F}~C_{M}U_{F}~C_{M}U_{F}~G_{M}G_{F}~U_{M}C_{F}~C_{M}U_{F}~U_{M}~A_{F}~C_{M}~U_{F}~U_{M}~Aₘₒₑ~Aₘₒₑ
P-Tₘₒₑ~U_{F}~G_{M}U_{F}~C_{M}U_{F}~C_{M}U_{F}~G_{M}G_{F}~U_{M}C_{F}~C_{M}U_{F}~U_{M}~A_{F}~C_{M}~U_{F}~U_{M}~Aₘₒₑ~Aₘₒₑ
P-U_{M}~U_{F}G_{M}U_{F}C_{M}U_{F}C_{M}U_{F}G_{M}G_{F}U_{M}C_{F}~C_{M}~U_{F}~U_{M}~A_{F}~C_{M}~U_{F}~U_{M}~Aₘₒₑ~Aₘₒₑ

Any of the 5′-end nucleosides (P-U_{F} or P-U_{M}) shown in the examples of agsRNAi compounds in Tables 8A and 8B can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{F}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 8A or 8B with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the nucleosides in Tables 8A or 8B with an L modification found in the overhang precursor can have the L replaced with any of those units and linkages provided for herein for overhang precursors including one or more of the sugar, sugar analogs or sugar substitutes independently selected from the group consisting of: F, J, W, V, Y, T, TL and I.

Any of the compounds in Tables 8A and 8B can any of those units and linkages provided for herein for overhang precursors.

B. Examples of agsRNAi Compounds:

TABLE 8A
Composition of Matter
Preferred use In Vitro or with a Protective Carrier In Vivo
(5′-3′)

P-U_{F}~U_{M}~G_{F}U_{F12}~C_{F}U_{F}~C_{F4}U_{F12}~G_{F}G_{F}U_{F}~C_{F}~C_{F4}UU_{F12}~A_{F}~C_{F}U_{F}U_{F}~A_{F}~G_{F}
P-U_{F}~U_{M}~G_{F}U_{F3}~C_{F}U_{F}~C_{F4}U_{F3}~G_{F}G_{F}U_{F}~C_{F}~C_{F4}UU_{F3}~A_{F}~C_{F}U_{F}U_{F}~A_{F}~G_{F}
P-U_{F}~U_{M}~G_{F}U_{F18}~C_{F}U_{F}~C_{F4}U_{F18}~G_{F}G_{F}U_{F}~C_{F}~C_{F4}UU_{F18}~A_{F}~C_{F}U_{F}U_{F}~A_{F}~G_{F}
P-U_{F}~U_{M}~G_{F}U_{F12}~C_{F4}U_{F}~C_{F}U_{F12}~G_{F}G_{F}U_{F}~C_{F4}~C_{F}UU_{F12}~A_{F}~C_{F4}U_{F}U_{F}~A_{F}~G_{F}
P-U_{F}~U_{M}~G_{F}U_{F3}~C_{F4}U_{F}~C_{F}U_{F3}~G_{F}G_{F}U_{F}~C_{F4}~C_{F}UU_{F3}~A_{F}~C_{F4}U_{F}U_{F}~A_{F}~G_{F}
P-U_{F}~U_{M}~G_{F}U_{F18}~C_{F4}U_{F}~C_{F}U_{F18}~G_{F}G_{F}U_{F}~C_{F4}~C_{F}UU_{F18}~A_{F}~C_{F4}U_{F}U_{F}~A_{F}~G_{F}
P-U_{F}~U_{M}~G_{F}U_{F12}~C_{F4}U_{F}~C_{F4}U_{F12}~G_{F}G_{F}U_{F}~C_{F4}~C_{F4}UU_{F12}~A_{F}~C_{F4}U_{F}U_{F}~A_{F}~G_{F}
P-U_{F}~U_{M}~G_{F}U_{F3}~C_{F4}U_{F}~C_{F4}U_{F3}~G_{F}G_{F}U_{F}~C_{F4}~C_{F4}UU_{F3}~A_{F}~C_{F4}U_{F}U_{F}~A_{F}~G_{F}
P-U_{F}~U_{M}~G_{F}U_{F18}~C_{F4}U_{F}~C_{F4}U_{F18}~G_{F}G_{F}U_{F}~C_{F4}~C_{F4}UU_{F18}~A_{F}~C_{F4}U_{F}U_{F}~A_{F}~G_{F}
P-U_{F}~U_{M}~G_{F}U_{F12}~C_{F}U_{F}~C_{F}U_{F12}~G_{F}G_{F}U_{F12}~C_{F}~C_{F}UU_{F12}~A_{F}~C_{F}U_{F}U_{F}~A_{F}~G_{F}
P-U_{F}~U_{M}~G_{F}U_{F3}~C_{F}U_{F}~C_{F}U_{F3}~G_{F}G_{F}U_{F3}~C_{F}~C_{F}UU_{F3}~A_{F}~C_{F}U_{F}U_{F}~A_{F}~G_{F}
P-U_{F}~U_{M}~G_{F}U_{F18}~C_{F}U_{F}~C_{F}U_{F18}~G_{F}G_{F}U_{F18}~C_{F}~C_{F}UU_{F18}~A_{F}~C_{F}U_{F}U_{F}~A_{F}~G_{F}
P-U_{F}~U_{M}~G_{F}U_{F12}~C_{F}U_{F12}~C_{F}U_{F12}~G_{F}G_{F}U_{F12}~C_{F}~C_{F}UU_{F12}~A_{F}~C_{F}U_{F}U_{F}~A_{F}~G_{F}
P-U_{F}~U_{M}~G_{F}U_{F3}~C_{F}U_{F3}~C_{F}U_{F3}~G_{F}G_{F}U_{F3}~C_{F}~C_{F}UU_{F3}~A_{F}~C_{F}U_{F}U_{F}~A_{F}~G_{F}
P-U_{F}~U_{M}~G_{F}U_{F18}~C_{F}U_{F18}~C_{F}U_{F18}~G_{F}G_{F}U_{F18}~C_{F}~C_{F}UU_{F18}~A_{F}~C_{F}U_{F}U_{F}~A_{F}~G_{F}
P-U_{F}~U_{M}~G_{F}U_{F12}~C_{F}U_{F12}~C_{F}U_{F12}~G_{F}G_{F}U_{F12}~C_{F}~C_{F}UU_{F12}~A_{F}~C_{F}U_{F12}U_{F}~A_{F}~G_{F}
P-U_{F}~U_{M}~G_{F}U_{F3}~C_{F}U_{F3}~C_{F}U_{F3}~G_{F}G_{F}U_{F3}~C_{F}~C_{F}UU_{F3}~A_{F}~C_{F}U_{F3}U_{F}~A_{F}~G_{F}
P-U_{F}~U_{M}~G_{F}U_{F18}~C_{F}U_{F18}~C_{F}U_{F18}~G_{F}G_{F}U_{F18}~C_{F}~C_{F}UU_{F18}~A_{F}~C_{F}U_{F18}U_{F}~A_{F}~G_{F}
P-U_{F}~U_{M}~G_{F}U_{F12}~C_{F}U_{F}~C_{F4}U_{F12}~G_{F}G_{F}U_{F12}~C_{F4}~C_{F}UU_{F12}~A_{F}~C_{F4}U_{F}U_{F}~A_{F}~G_{F}
P-U_{F}~U_{M}~G_{F}U_{F3}~C_{F}U_{F}~C_{F4}U_{F3}~G_{F}G_{F}U_{F3}~C_{F4}~C_{F}UU_{F3}~A_{F}~C_{F4}U_{F}U_{F}~A_{F}~G_{F}
P-U_{F}~U_{M}~G_{F}U_{F18}~C_{F}U_{F}~C_{F4}U_{F18}~G_{F}G_{F}U_{F18}~C_{F4}~C_{F}UU_{F18}~A_{F}~C_{F4}U_{F}U_{F}~A_{F}~G_{F}
P-U_{F}~U_{M}~G_{F}U_{F12}~C_{F}U_{F12}~C_{F4}U_{F12}~G_{F}G_{F}U_{F12}~C_{F4}~C_{F}UU_{F12}~A_{F}~C_{F4}U_{F}U_{F}~A_{F}~G_{F}
P-U_{F}~U_{M}~G_{F}U_{F3}~C_{F}U_{F3}~C_{F4}U_{F3}~G_{F}G_{F}U_{F3}~C_{F4}~C_{F}UU_{F3}~A_{F}~C_{F4}U_{F}U_{F}~A_{F}~G_{F}
P-U_{F}~U_{M}~G_{F}U_{F18}~C_{F}U_{F18}~C_{F4}U_{F18}~G_{F}G_{F}U_{F18}~C_{F4}~C_{F}UU_{F18}~A_{F}~C_{F4}U_{F}U_{F}~A_{F}~G_{F}
P-U_{F}~U_{M}~G_{F}U_{F12}~C_{F}U_{F12}~C_{F4}U_{F12}~G_{F}G_{F}U_{F12}~C_{F4}~C_{F}UU_{F12}~A_{F}~C_{F4}U_{F12}U_{F}~A_{F}~G_{F}
P-U_{F}~U_{M}~G_{F}U_{F3}~C_{F}U_{F3}~C_{F4}U_{F3}~G_{F}G_{F}U_{F3}~C_{F4}~C_{F}UU_{F3}~A_{F}~C_{F4}U_{F3}U_{F}~A_{F}~G_{F}
P-U_{F}~U_{M}~G_{F}U_{F18}~C_{F}U_{F18}~C_{F4}U_{F18}~G_{F}G_{F}U_{F18}~C_{F4}~C_{F}UU_{F18}~A_{F}~C_{F4}U_{F18}U_{F}~A_{F}~G_{F}
P-U_{F}~U_{M}~G_{F}U_{F12}~C_{F4}U_{F}~C_{F4}U_{F12}~G_{F}G_{F}U_{F12}~C_{F4}~C_{F4}UU_{F12}~A_{F}~C_{F4}U_{F}U_{F}~A_{F}~G_{F}
P-U_{F}~U_{M}~G_{F}U_{F3}~C_{F4}U_{F}~C_{F4}U_{F3}~G_{F}G_{F}U_{F3}~C_{F4}~C_{F4}UU_{F3}~A_{F}~C_{F4}U_{F}U_{F}~A_{F}~G_{F}
P-U_{F}~U_{M}~G_{F}U_{F18}~C_{F4}U_{F}~C_{F4}U_{F18}~G_{F}G_{F}U_{F18}~C_{F4}~C_{F4}UU_{F18}~A_{F}~C_{F4}U_{F}U_{F}~A_{F}~G_{F}
P-U_{F}~U_{M}~G_{F}U_{F12}~C_{F4}U_{F12}~C_{F4}U_{F12}~G_{F}G_{F}U_{F12}~C_{F4}~C_{F4}UU_{F12}~A_{F}~C_{F4}U_{F}U_{F}~A_{F}~G_{F}
P-U_{F}~U_{M}~G_{F}U_{F3}~C_{F4}U_{F3}~C_{F4}U_{F3}~G_{F}G_{F}U_{F3}~C_{F4}~C_{F4}UU_{F3}~A_{F}~C_{F4}U_{F}U_{F}~A_{F}~G_{F}
P-U_{F}~U_{M}~G_{F}U_{F18}~C_{F4}U_{F18}~C_{F4}U_{F18}~G_{F}G_{F}U_{F18}~C_{F4}~C_{F4}UUFIG~A_{F}~C_{F4}U_{F}U_{F}~A_{F}~G_{F}
P-U_{F}~U_{M}~G_{F}U_{F12}~C_{F4}U_{F12}~C_{F4}U_{F12}~G_{F}G_{F}U_{F12}~C_{F4}~C_{F4}UU_{F12}~A_{F}~C_{F4}U_{F12}U_{F}~A_{F}~G_{F}
P-U_{F}~U_{M}~G_{F}U_{F3}~C_{F4}U_{F3}~C_{F4}U_{F3}~G_{F}G_{F}U_{F3}~C_{F4}~C_{F4}UU_{F3}~A_{F}~C_{F4}U_{F3}U_{F}~A_{F}~G_{F}
P-U_{F}~U_{M}~G_{F}U_{F18}~C_{F4}U_{F18}~C_{F4}U_{F18}~G_{F}G_{F}U_{F18}~C_{F4}~C_{F4}UU_{F18}~A_{F}~C_{F4}U_{F18}U_{F}~A_{F}~G_{F}
P-U_{F}~U_{M}~G_{F}U_{F}~C_{F4}U_{F}~C_{F4}A_{F}~G_{F}G_{F}U_{F}~C_{F4}~C_{F4}UU_{F}~A_{F}~C_{F4}U_{F}U_{F}~A_{F}~G_{F}
P-U_{F}~U_{M}~G_{F}U_{F}~C_{F4}U_{F}~C_{F4}U_{F}~A_{F}G_{F}U_{F}~C_{F4}~C_{F4}UU_{F}~A_{F}~C_{F4}U_{F}U_{F}~A_{F}~G_{F}
P-U_{F}~U_{M}~G_{L}U_{F}~C_{F}U_{L}C_{F}U_{F}~G_{F}G_{F}U_{F}~C_{L}~C_{F}UU_{F}~A_{L}~C_{F}U_{L}U_{F}~A_{F}~G_{F}
P-U_{F}~U_{M}~G_{L}U_{F12}~C_{F}U_{L}C_{F4}U_{F12}~G_{F}G_{F}U_{F}~C_{L}~C_{F4}UU_{F12}~A_{L}~C_{F}U_{L}U_{F}~A_{F}~G_{F}
P-U_{F}~U_{M}~G_{L}U_{F3}~C_{F}U_{L}C_{F4}U_{F3}~G_{F}G_{F}U_{F}~C_{L}~C_{F4}UU_{F3}~A_{L}~C_{F}U_{L}U_{F}~A_{F}~G_{F}
P-U_{F}~U_{M}~G_{L}U_{F18}~C_{F}U_{L}C_{F4}U_{F18}~G_{F}G_{F}U_{F}~C_{L}~C_{F4}UU_{F18}~A_{L}~C_{F}U_{L}U_{F}~A_{F}~G_{F}
P-U_{F}~U_{M}~G_{L}U_{F12}~C_{F4}U_{L}C_{F}U_{F12}~G_{F}G_{F}U_{F}~C_{L4}~C_{F}UU_{F12}~A_{L}~C_{F4}U_{L}U_{F}~A_{F}~G_{F}
P-U_{F}~U_{M}~G_{L}U_{F3}~C_{F4}U_{L}C_{F}U_{F3}~G_{F}G_{F}U_{F}~C_{L4}~C_{F}UU_{F3}~A_{L}~C_{F4}U_{L}U_{F}~A_{F}~G_{F}
P-U_{F}~U_{M}~G_{L}U_{F18}~C_{F4}U_{L}C_{F}U_{F18}~G_{F}G_{F}U_{F}~C_{L4}~C_{F}UU_{F18}~A_{L}~C_{F4}U_{L}U_{F}~A_{F}~G_{F}
P-U_{F}~U_{M}~G_{L}U_{F12}~C_{F4}U_{L}C_{F4}U_{F12}~G_{F}G_{F}U_{F}~C_{L4}~C_{F4}UU_{F12}~A_{L}~C_{F4}U_{L}U_{F}~A_{F}~G_{F}
P-U_{F}~U_{M}~G_{L}U_{F3}~C_{F4}U_{L}C_{F4}U_{F3}~G_{F}G_{F}U_{F}~C_{L4}~C_{F4}UU_{F3}~A_{L}~C_{F4}U_{L}U_{F}~A_{F}~G_{F}
P-U_{F}~U_{M}~G_{L}U_{F18}~C_{F4}U_{L}C_{F4}U_{F18}~G_{F}G_{F}U_{F}~C_{L4}~C_{F4}UU_{F18}~A_{L}~C_{F4}U_{L}U_{F}~A_{F}~G_{F}
P-U_{F}~U_{M}~G_{L}U_{F12}~C_{F}U_{L}C_{F}U_{F12}~G_{F}G_{F}U_{F12}~C_{L}~C_{F}UU_{F12}~A_{L}~C_{F}U_{L}U_{F}~A_{F}~G_{F}
P-U_{F}~U_{M}~G_{L}U_{F3}~C_{F}U_{L}C_{F}U_{F3}~G_{F}G_{F}U_{F3}~C_{L}~C_{F}UU_{F3}~A_{L}~C_{F}U_{L}U_{F}~A_{F}~G_{F}
P-U_{F}~U_{M}~G_{L}U_{F18}~C_{F}U_{L}C_{F}U_{F18}~G_{F}G_{F}U_{F18}~C_{L}~C_{F}UU_{F18}~A_{L}~C_{F}U_{L}U_{F}~A_{F}~G_{F}
P-U_{F}~U_{M}~G_{L}U_{F12}~C_{F}U_{L12}~C_{F}U_{F12}~G_{F}G_{F}U_{F12}~C_{L}~C_{F}UU_{F12}~A_{L}~C_{F}U_{L}U_{F}~A_{F}~G_{F}
P-U_{F}~U_{M}~G_{L}U_{F3}~C_{F}U_{L3}~C_{F}U_{F3}~G_{F}G_{F}U_{F3}~C_{L}~C_{F}UU_{F3}~A_{L}~C_{F}U_{L}U_{F}~A_{F}~G_{F}
P-U_{F}~U_{M}~G_{L}U_{F18}~C_{F}U_{L18}~C_{F}U_{F18}~G_{F}G_{F}U_{F18}~C_{L}~C_{F}UU_{F18}~A_{L}~C_{F}U_{L}U_{F}~A_{F}~G_{F}
P-U_{F}~U_{M}~G_{L}U_{F12}~C_{F}U_{L12}~C_{F}U_{F12}~G_{F}G_{F}U_{F12}~C_{L}~C_{F}UU_{F12}~A_{L}~C_{F}U_{L12}U_{F}~A_{F}~G_{F}
P-U_{F}~U_{M}~G_{L}U_{F3}~C_{F}U_{L3}~C_{F}U_{F3}~G_{F}G_{F}U_{F3}~C_{L}~C_{F}UU_{F3}~A_{L}~C_{F}U_{L3}U_{F}~A_{F}~G_{F}
P-U_{F}~U_{M}~G_{L}U_{F18}~C_{F}U_{L18}~C_{F}U_{F18}~G_{F}G_{F}U_{F18}~C_{L}~C_{F}UU_{F18}~A_{L}~C_{F}U_{L18}U_{F}~A_{F}~G_{F}
P-U_{F}~U_{M}~G_{L}U_{F12}~C_{F}U_{L}~C_{F4}U_{F12}~G_{F}G_{F}U_{F12}~C_{L4}~C_{F}UU_{F12}~A_{L}~C_{F4}U_{L}U_{F}~A_{F}~G_{F}
P-U_{F}~U_{M}~G_{L}U_{F3}~C_{F}U_{L}~C_{F4}U_{F3}~G_{F}G_{F}U_{F3}~C_{L4}~C_{F}UU_{F3}~A_{L}~C_{F4}U_{L}U_{F}~A_{F}~G_{F}
P-U_{F}~U_{M}~G_{L}U_{F18}~C_{F}U_{L}~C_{F4}U_{F18}~G_{F}G_{F}U_{F18}~C_{L4}~C_{F}UU_{F18}~A_{L}~C_{F4}U_{L}U_{F}~A_{F}~G_{F}
P-U_{F}~U_{M}~G_{L}U_{F12}~C_{F}U_{L12}~C_{F4}U_{F12}~G_{F}G_{F}U_{F12}~C_{L4}~C_{F}UU_{F12}~A_{L}~C_{F4}U_{L}U_{F}~A_{F}~G_{F}
P-U_{F}~U_{M}~G_{L}U_{F3}~C_{F}U_{L3}~C_{F4}U_{F3}~G_{F}G_{F}U_{F3}~C_{L4}~C_{F}UU_{F3}~A_{L}~C_{F4}U_{L}U_{F}~A_{F}~G_{F}
P-U_{F}~U_{M}~G_{L}U_{F18}~C_{F}U_{L18}~C_{F4}U_{F18}~G_{F}G_{F}U_{F18}~C_{L4}~C_{F}UU_{F18}~A_{L}~C_{F4}U_{L}U_{F}~A_{F}~G_{F}
P-U_{F}~U_{M}~G_{L}U_{F12}~C_{F}U_{L12}~C_{F4}U_{F12}~G_{F}G_{F}U_{F12}~C_{L4}~C_{F}UU_{F12}~A_{L}~C_{F4}U_{L12}U_{F}~A_{F}~G_{F}
P-U_{F}~U_{M}~G_{L}U_{F3}~C_{F}U_{L3}~C_{F4}U_{F3}~G_{F}G_{F}U_{F3}~C_{L4}~C_{F}UU_{F3}~A_{L}~C_{F4}U_{L3}U_{F}~A_{F}~G_{F}
P-U_{F}~U_{M}~G_{L}U_{F18}~C_{F}U_{L18}~C_{F4}U_{F18}~G_{F}G_{F}U_{F18}~C_{L4}~C_{F}UU_{F18}~A_{L}~C_{F4}U_{L18}U_{F}~A_{F}~G_{F}
P-U_{F}~U_{M}~G_{L}U_{F12}~C_{F4}U_{L}C_{F4}U_{F12}~G_{F}G_{F}U_{F12}~C_{L4}~C_{F4}UU_{F12}~A_{L}~C_{F4}U_{L}U_{F}~A_{F}~G_{F}
P-U_{F}~U_{M}~G_{L}U_{F3}~C_{F4}U_{L}C_{F4}U_{F3}~G_{F}G_{F}U_{F3}~C_{L4}~C_{F4}UU_{F3}~A_{L}~C_{F4}U_{L}U_{F}~A_{F}~G_{F}
P-U_{F}~U_{M}~G_{L}U_{F18}~C_{F4}U_{L}C_{F4}U_{F18}~G_{F}G_{F}U_{F18}~C_{L4}~C_{F4}UU_{F18}~A_{L}~C_{F4}U_{L}U_{F}~A_{F}~G_{F}
P-U_{F}~U_{M}~G_{L}U_{F12}~C_{F4}U_{L12}~C_{F4}U_{F12}~G_{F}G_{F}U_{F12}~C_{L4}~C_{F4}UU_{F12}~A_{L}~C_{F4}U_{L}U_{F}~A_{F}~G_{F}
P-U_{F}~U_{M}~G_{L}U_{F3}~C_{F4}U_{L3}~C_{F4}U_{F3}~G_{F}G_{F}U_{F3}~C_{L4}~C_{F4}UU_{F3}~A_{L}~C_{F4}U_{L}U_{F}~A_{F}~G_{F}
P-U_{F}~U_{M}~G_{L}U_{F18}~C_{F4}U_{L18}~C_{F4}U_{F18}~G_{F}G_{F}U_{F18}~C_{L4}~C_{F4}UU_{F18}~A_{L}~C_{F4}U_{L}U_{F}~A_{F}~G_{F}
P-U_{F}~U_{M}~G_{L}U_{F12}~C_{F4}U_{L12}~C_{F4}U_{F12}~G_{F}G_{F}U_{F12}~C_{L4}~C_{F4}UU_{F12}~A_{L}~C_{F4}U_{L12}U_{F}~A_{F}~G_{F}
P-U_{F}~U_{M}~G_{L}U_{F3}~C_{F4}U_{L3}~C_{F4}U_{F3}~G_{F}G_{F}U_{F3}~C_{L4}~C_{F4}UU_{F3}~A_{L}~C_{F4}U_{L3}U_{F}~A_{F}~G_{F}
P-U_{F}~U_{M}~G_{L}U_{F18}~C_{F4}U_{L18}~C_{F4}U_{F18}~G_{F}G_{F}U_{F18}~C_{L4}~C_{F4}UU_{F18}~A_{L}~C_{F4}U_{L18}U_{F}~A_{F}~G_{F}
P-U_{F}~U_{M}~G_{L}U_{F}~C_{F4}U_{L}C_{F4}A_{F}~G_{F}G_{F}U_{F}~C_{L4}~C_{F4}UU_{F}~A_{L}~C_{F4}U_{L}U_{F}~A_{F}~G_{F}
P-U_{F}~U_{M}~G_{L}U_{F}~C_{F4}U_{L}C_{F4}U_{F}~A_{F}G_{F}U_{F}~C_{L4}~C_{F4}UU_{F}~A_{L}~C_{F4}U_{L}U_{F}~A_{F}~G_{F}
P-U_{F}~U_{M}~G_{F}U_{F}~C_{L}U_{F}~C_{F}U_{F}~G_{L}G_{F}U_{F}~C_{L}~C_{F}UU_{F}~A_{F}~C_{L}U_{F}U_{F}~A_{F}~G_{F}
P-U_{F}~U_{M}~G_{F}U_{F12}~C_{L}U_{F}~C_{F4}U_{F12}~G_{L}G_{F}U_{F}~C_{L}~C_{F4}UU_{F12}~A_{F}~C_{L}U_{F}U_{F}~A_{F}~G_{F}
P-U_{F}~U_{M}~G_{F}U_{F3}~C_{L}U_{F}~C_{F4}U_{F3}~G_{L}G_{F}U_{F}~C_{L}~C_{F4}UU_{F3}~A_{F}~C_{L}U_{F}U_{F}~A_{F}~G_{F}
P-U_{F}~U_{M}~G_{F}U_{F18}~C_{L}U_{F}~C_{F4}U_{F18}~G_{L}G_{F}U_{F}~C_{L}~C_{F4}UU_{F18}~A_{F}~C_{L}U_{F}U_{F}~A_{F}~G_{F}
P-U_{F}~U_{M}~G_{F}U_{F12}~C_{L}U_{F}~C_{F}U_{F12}~G_{L}G_{F}U_{F}~C_{L4}~C_{F}UU_{F12}~A_{F}~C_{L4}U_{F}U_{F}~A_{F}~G_{F}
P-U_{F}~U_{M}~G_{F}U_{F3}~C_{L}U_{F}~C_{F}U_{F3}~G_{L}G_{F}U_{F}~C_{L4}~C_{F}UU_{F3}~A_{F}~C_{L4}U_{F}U_{F}~A_{F}~G_{F}
P-U_{F}~U_{M}~G_{F}U_{F18}~C_{L}U_{F}~C_{F}U_{F18}~G_{L}G_{F}U_{F}~C_{L4}~C_{F}UU_{F18}~A_{F}~C_{L4}U_{F}U_{F}~A_{F}~G_{F}
P-U_{F}~U_{M}~G_{F}U_{F12}~C_{L}U_{F}~C_{F4}U_{F12}~G_{L}G_{F}U_{F}~C_{L4}~C_{F4}UU_{F12}~A_{F}~C_{L4}U_{F}U_{F}~A_{F}~G_{F}
P-U_{F}~U_{M}~G_{F}U_{F3}~C_{L}U_{F}~C_{F4}U_{F3}~G_{L}G_{F}U_{F}~C_{L4}~C_{F4}UU_{F3}~A_{F}~C_{L4}U_{F}U_{F}~A_{F}~G_{F}
P-U_{F}~U_{M}~G_{F}U_{F18}~C_{L}U_{F}~C_{F4}U_{F18}~G_{L}G_{F}U_{F}~C_{L4}~C_{F4}UU_{F18}~A_{F}~C_{L4}U_{F}U_{F}~A_{F}~G_{F}
P-U_{F}~U_{M}~G_{F}U_{F12}~C_{L}U_{F}~C_{F}U_{F12}~G_{L}G_{F}U_{F12}~C_{L}~C_{F}UU_{F12}~A_{F}~C_{L}U_{F}U_{F}~A_{F}~G_{F}
P-U_{F}~U_{M}~G_{F}U_{F3}~C_{L}U_{F}~C_{F}U_{F3}~G_{L}G_{F}U_{F3}~C_{L}~C_{F}UU_{F3}~A_{F}~C_{L}U_{F}U_{F}~A_{F}~G_{F}
P-U_{F}~U_{M}~G_{F}U_{F18}~C_{L}U_{F}~C_{F}U_{F18}~G_{L}G_{F}U_{F18}~C_{L}~C_{F}UU_{F18}~A_{F}~C_{L}U_{F}U_{F}~A_{F}~G_{F}
P-U_{F}~U_{M}~G_{F}U_{F12}~C_{L}U_{F12}~C_{F}U_{F12}~G_{L}G_{F}U_{F12}~C_{L}~C_{F}UU_{F12}~A_{F}~C_{L}U_{F}U_{F}~A_{F}~G_{F}
P-U_{F}~U_{M}~G_{F}U_{F3}~C_{L}U_{F3}~C_{F}U_{F3}~G_{L}G_{F}U_{F3}~C_{L}~C_{F}UU_{F3}~A_{F}~C_{L}U_{F}U_{F}~A_{F}~G_{F}
P-U_{F}~U_{M}~G_{F}U_{F18}~C_{L}U_{F18}~C_{F}U_{F18}~G_{L}G_{F}U_{F18}~C_{L}~C_{F}UU_{F18}~A_{F}~C_{L}U_{F}U_{F}~A_{F}~G_{F}
P-U_{F}~U_{M}~G_{F}U_{F12}~C_{L}U_{F12}~C_{F}U_{F12}~G_{L}G_{F}U_{F12}~C_{L}~C_{F}UU_{F12}~A_{F}~C_{L}U_{F12}U_{F}~A_{F}~G_{F}
P-U_{F}~U_{M}~G_{F}U_{F3}~C_{L}U_{F3}~C_{F}U_{F3}~G_{L}G_{F}U_{F3}~C_{L}~C_{F}UU_{F3}~A_{F}~C_{L}U_{F3}U_{F}~A_{F}~G_{F}
P-U_{F}~U_{M}~G_{F}U_{F18}~C_{L}U_{F18}~C_{F}U_{F18}~G_{L}G_{F}U_{F18}~C_{L}~C_{F}UU_{F18}~A_{F}~C_{L}U_{F18}U_{F}~A_{F}~G_{F}
P-U_{F}~U_{M}~G_{F}U_{F12}~C_{L}U_{F}~C_{F4}U_{F12}~G_{L}G_{F}U_{F12}~C_{L4}~C_{F}UU_{F12}~A_{F}~C_{L4}U_{F}U_{F}~A_{F}~G_{F}
P-U_{F}~U_{M}~G_{F}U_{F3}~C_{L}U_{F}~C_{F4}U_{F3}~G_{L}G_{F}U_{F3}~C_{L4}~C_{F}UU_{F3}~A_{F}~C_{L4}U_{F}U_{F}~A_{F}~G_{F}
P-U_{F}~U_{M}~G_{F}U_{F18}~C_{L}U_{F}~C_{F4}U_{F18}~G_{L}G_{F}U_{F18}~C_{L4}~C_{F}UU_{F18}~A_{F}~C_{L4}U_{F}U_{F}~A_{F}~G_{F}
P-U_{F}~U_{M}~G_{F}U_{F12}~C_{L}U_{F12}~C_{F4}U_{F12}~G_{L}G_{F}U_{F12}~C_{L4}~C_{F}UU_{F12}~A_{F}~C_{L4}U_{F}U_{F}~A_{F}~G_{F}
P-U_{F}~U_{M}~G_{F}U_{F3}~C_{L}U_{F3}~C_{F4}U_{F3}~G_{L}G_{F}U_{F3}~C_{L4}~C_{F}UU_{F3}~A_{F}~C_{L4}U_{F}U_{F}~A_{F}~G_{F}
P-U_{F}~U_{M}~G_{F}U_{F18}~C_{L}U_{F18}~C_{F4}U_{F18}~G_{L}G_{F}U_{F18}~C_{L4}~C_{F}UU_{F18}~A_{F}~C_{L4}U_{F}U_{F}~A_{F}~G_{F}
P-U_{F}~U_{M}~G_{F}U_{F12}~C_{L}U_{F12}~C_{F4}U_{F12}~G_{L}G_{F}U_{F12}~C_{L4}~C_{F}UU_{F12}~A_{F}~C_{L4}U_{F12}U_{F}~A_{F}~G_{F}
P-U_{F}~U_{M}~G_{F}U_{F3}~C_{L}U_{F3}~C_{F4}U_{F3}~G_{L}G_{F}U_{F3}~C_{L4}~C_{F}UU_{F3}~A_{F}~C_{L4}U_{F3}U_{F}~A_{F}~G_{F}
P-U_{F}~U_{M}~G_{F}U_{F18}~C_{L}U_{F18}~C_{F4}U_{F18}~G_{L}G_{F}U_{F18}~C_{L4}~C_{F}UU_{F18}~A_{F}~C_{L4}U_{F18}U_{F}~A_{F}~G_{F}
P-U_{F}~U_{M}~G_{F}U_{F12}~C_{L4}U_{F}~C_{F4}U_{F12}~G_{L}G_{F}U_{F12}~C_{L4}~C_{F4}UU_{F12}~A_{F}~C_{L4}U_{F}U_{F}~A_{F}~G_{F}
P-U_{F}~U_{M}~G_{F}U_{F3}~C_{L4}U_{F}~C_{F4}U_{F3}~G_{L}G_{F}U_{F3}~C_{L4}~C_{F4}UU_{F3}~A_{F}~C_{L4}U_{F}U_{F}~A_{F}~G_{F}
P-U_{F}~U_{M}~G_{F}U_{F18}~C_{L4}U_{F}~C_{F4}U_{F18}~G_{L}G_{F}U_{F18}~C_{L4}~C_{F4}UU_{F18}~A_{F}~C_{L4}U_{F}U_{F}~A_{F}~G_{F}
P-U_{F}~U_{M}~G_{F}U_{F12}~C_{L4}U_{F12}~C_{F4}U_{F12}~G_{L}G_{F}U_{F12}~C_{L4}~C_{F4}UU_{F12}~A_{F}~C_{L4}U_{F}U_{F}~A_{F}~G_{F}
P-U_{F}~U_{M}~G_{F}U_{F3}~C_{L4}U_{F3}~C_{F4}U_{F3}~G_{L}G_{F}U_{F3}~C_{L4}~C_{F4}UU_{F3}~A_{F}~C_{L4}U_{F}U_{F}~A_{F}~G_{F}
P-U_{F}~U_{M}~G_{F}U_{F18}~C_{L4}U_{F18}~C_{F4}U_{F18}~G_{L}G_{F}U_{F18}~C_{L4}~C_{F4}UU_{F18}~A_{F}~C_{L4}U_{F}U_{F}~A_{F}~G_{F}
P-U_{F}~U_{M}~G_{F}U_{F12}~C_{L4}U_{F12}~C_{F4}U_{F12}~G_{L}G_{F}U_{F12}~C_{L4}~C_{F4}UU_{F12}~A_{F}~C_{L4}U_{F12}U_{F}~A_{F}~G_{F}
P-U_{F}~U_{M}~G_{F}U_{F3}~C_{L4}U_{F3}~C_{F4}U_{F3}~G_{L}G_{F}U_{F3}~C_{L4}~C_{F4}UU_{F3}~A_{F}~C_{L4}U_{F3}U_{F}~A_{F}~G_{F}
P-U_{F}~U_{M}~G_{F}U_{F18}~C_{L4}U_{F18}~C_{F4}U_{F18}~G_{L}G_{F}U_{F18}~C_{L4}~C_{F4}UU_{F18}~A_{F}~C_{L4}U_{F18}U_{F}~A_{F}~G_{F}
P-U_{F}~U_{M}~G_{F}U_{F}~C_{L4}U_{F}~C_{F4}A_{F}~G_{L}G_{F}U_{F}~C_{L4}~C_{F4}UU_{F}~A_{F}~C_{L4}U_{F}U_{F}~A_{F}~G_{F}
P-U_{F}~U_{M}~G_{F}U_{F}~C_{L4}U_{F}~C_{F4}U_{F}~A_{L}G_{F}U_{F}~C_{L4}~C_{F4}UU_{F}~A_{F}~C_{L4}U_{F}U_{F}~A_{F}~G_{F}
P-U_{F}~U_{M}~G_{F}U_{L}C_{F}U_{F}~C_{F}U_{L}G_{F}G_{F}U_{F}~C_{F}~C_{F}UU_{L}~A_{F}~C_{F}U_{L}U_{F}~A_{F}~G_{F}
P-U_{F}~U_{M}~G_{F}U_{L}~C_{F}U_{F}~C_{F4}U_{L}~G_{F}G_{F}U_{F}~C_{F}~C_{F4}UU_{L}~A_{F}~C_{F}U_{L}U_{F}~A_{F}~G_{F}
P-U_{F}~U_{M}~G_{F}U_{L}~C_{F}U_{F}~C_{F4}U_{L}~G_{F}G_{F}U_{F}~C_{F}~C_{F4}UU_{L}~A_{F}~C_{F}U_{L}U_{F}~A_{F}~G_{F}
P-U_{F}~U_{M}~G_{F}U_{L}~C_{F}U_{F}~C_{F4}U_{L}~G_{F}G_{F}U_{F}~C_{F}~C_{F4}UU_{L}~A_{F}~C_{F}U_{L}U_{F}~A_{F}~G_{F}
P-U_{F}~U_{M}~G_{F}U_{L}~C_{F4}U_{F}~C_{F}U_{L}~G_{F}G_{F}U_{F}~C_{F4}~C_{F}UU_{L}~A_{F}~C_{F4}U_{L}U_{F}~A_{F}~G_{F}
P-U_{F}~U_{M}~G_{F}U_{L}~C_{F4}U_{F}~C_{F}U_{L}~G_{F}G_{F}U_{F}~C_{F4}~C_{F}UU_{L}~A_{F}~C_{F4}U_{L}U_{F}~A_{F}~G_{F}
P-U_{F}~U_{M}~G_{F}U_{L}~C_{F4}U_{F}~C_{F}U_{L}~G_{F}G_{F}U_{F}~C_{F4}~C_{F}UU_{L}~A_{F}~C_{F4}U_{L}U_{F}~A_{F}~G_{F}
P-U_{F}~U_{M}~G_{F}U_{L}~C_{F4}U_{F}~C_{F4}U_{L}~G_{F}G_{F}U_{F}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F}~C_{F4}U_{L}~G_{F}G_{F}U_{F}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F}~C_{F4}U_{L}~G_{F}G_{F}U_{F}~C_{F4}~C_{F4}UU_{L}~A_{F}~C_{F4}U_{L}U_{F}~A_{F}~G_{F}
P-U_{F}~U_{M}~G_{F}U_{L}~C_{F}U_{F}~C_{F}U_{L12}~G_{F}G_{F}U_{F12}~C_{F}~C_{F}UU_{L}~A_{F}~C_{F}U_{L}U_{F}~A_{F}~G_{F}
P-U_{F}~U_{M}~G_{F}U_{L}~C_{F}U_{F}~C_{F}U_{L3}~G_{F}G_{F}U_{F3}~C_{F}~C_{F}UU_{L}~A_{F}~C_{F}U_{L}U_{F}~A_{F}~G_{F}
P-U_{F}~U_{M}~G_{F}U_{L}~C_{F}U_{F}~C_{F}U_{L18}~G_{F}G_{F}U_{F18}~C_{F}~C_{F}UU_{L}~A_{F}~C_{F}U_{L}U_{F}~A_{F}~G_{F}
P-U_{F}~U_{M}~G_{F}U_{L}~C_{F}U_{F12}~C_{F}U_{L12}~G_{F}G_{F}U_{F12}~C_{F}~C_{F}UU_{L}~A_{F}~C_{F}U_{L}U_{F}~A_{F}~G_{F}
P-U_{F}~U_{M}~G_{F}U_{L}~C_{F}U_{F3}~C_{F}U_{L3}~G_{F}G_{F}U_{F3}~C_{F}~C_{F}UU_{L}~A_{F}~C_{F}U_{L}U_{F}~A_{F}~G_{F}
P-U_{F}~U_{M}~G_{F}U_{L}~C_{F}U_{F18}~C_{F}U_{L18}~G_{F}G_{F}U_{F18}~C_{F}~C_{F}UU_{L}~A_{F}~C_{F}U_{L}U_{F}~A_{F}~G_{F}
P-U_{F}~U_{M}~G_{F}U_{L}~C_{F}U_{F12}~C_{F}U_{L12}~G_{F}G_{F}U_{F12}~C_{F}~C_{F}UU_{L}~A_{F}~C_{F}U_{L}U_{F}~A_{F}~G_{F}
P-U_{F}~U_{M}~G_{F}U_{L}~C_{F}U_{F3}~C_{F}U_{L3}~G_{F}G_{F}U_{F3}~C_{F}~C_{F}UU_{L}~A_{F}~C_{F}U_{L}U_{F}~A_{F}~G_{F}
P-U_{F}~U_{M}~G_{F}U_{L}~C_{F}U_{F18}~C_{F}U_{L18}~G_{F}G_{F}U_{F18}~C_{F}~C_{F}UU_{L}~A_{F}~C_{F}U_{L}U_{F}~A_{F}~G_{F}
P-U_{F}~U_{M}~G_{F}U_{L}~C_{F}U_{F}~C_{F4}U_{L12}~G_{F}G_{F}U_{F12}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F}~C_{F4}U_{L3}~G_{F}G_{F}U_{F3}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F}~C_{F4}U_{L18}~G_{F}G_{F}U_{F18}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F12}~C_{F4}U_{L12}~G_{F}G_{F}U_{F12}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F3}~C_{F4}U_{L3}~G_{F}G_{F}U_{F3}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F18}~C_{F4}U_{L18}~G_{F}G_{F}U_{F18}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F12}~C_{F4}U_{L12}~G_{F}G_{F}U_{F12}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F3}~C_{F4}U_{L3}~G_{F}G_{F}U_{F3}~C_{F4}~C_{F}UU_{L}~A_{F}~C_{F4}U_{L}U_{F}~A_{F}~G_{F}
P-U_{F}~U_{M}~G_{F}U_{L}~C_{F}U_{F18}~C_{F4}U_{L18}~G_{F}G_{F}U_{F18}~C_{F4}~C_{F}UU_{L}~A_{F}~C_{F4}U_{L}U_{F}~A_{F}~G_{F}
P-U_{F}~U_{M}~G_{F}U_{L}~C_{F4}U_{F}~C_{F4}U_{L12}~G_{F}G_{F}U_{F12}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F}~C_{F4}U_{L3}~G_{F}G_{F}U_{F3}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F}~C_{F4}U_{L18}~G_{F}G_{F}U_{F18}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F12}~C_{F4}U_{L12}~G_{F}G_{F}U_{F12}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F3}~C_{F4}U_{L3}~G_{F}G_{F}U_{F3}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F18}~C_{F4}U_{L18}~G_{F}G_{F}U_{F18}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F12}~C_{F4}U_{L12}~G_{F}G_{F}U_{F12}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F3}~C_{F4}U_{L3}~G_{F}G_{F}U_{F3}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F18}~C_{F4}U_{L18}~G_{F}G_{F}U_{F18}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F}~C_{F4}A_{L}~G_{F}G_{F}U_{F}~C_{F4}~C_{F4}UU_{L}~A_{F}~C_{F4}U_{L}U_{F}~A_{F}~G_{F}
P-U_{F}~U_{M}~G_{F}U_{L}~C_{F4}U_{F}~C_{F4}U_{L}~A_{F}G_{F}U_{F}~C_{F4}~C_{F4}UU_{L}~A_{F}~C_{F4}U_{L}U_{F}~A_{F}~G_{F}
P-U_{F}~U_{M}~G_{L}U_{F}~C_{F}U_{L}~C_{F}U_{F}~G_{L}G_{F}U_{F}~C_{F}~C_{F}UU_{L}~A_{F}~C_{L}U_{F}U_{F}~A_{F}~G_{F}
P-U_{F}~U_{M}~G_{L}U_{F12}~C_{F}U_{L}~C_{F4}U_{F12}~G_{L}G_{F}U_{F}~C_{F}~C_{F4}UU_{L}~A_{F}~C_{L}U_{F}U_{F}~A_{F}~G_{F}
P-U_{F}~U_{M}~G_{L}U_{F3}~C_{F}U_{L}~C_{F4}U_{F3}~G_{L}G_{F}U_{F}~C_{F}~C_{F4}UU_{L}~A_{F}~C_{L}U_{F}U_{F}~A_{F}~G_{F}
P-U_{F}~U_{M}~G_{L}U_{F18}~C_{F}U_{L}~C_{F4}U_{F18}~G_{L}G_{F}U_{F}~C_{F}~C_{F4}UU_{L}~A_{F}~C_{L}U_{L}U_{F}~A_{F}~G_{F}
P-U_{F}~U_{M}~G_{L}U_{F12}~C_{F4}U_{L}~C_{F}U_{F12}~G_{L}G_{F}U_{F}~C_{F4}~C_{F}UU_{L}~A_{F}~C_{L}U_{L}U_{F}~A_{F}~G_{F}
P-U_{F}~U_{M}~G_{L}U_{F3}~C_{F4}U_{L}~C_{F}U_{F3}~G_{L}G_{F}U_{F}~C_{F4}~C_{F}UU_{L}~A_{F}~C_{L}U_{L}U_{F}~A_{F}~G_{F}
P-U_{F}~U_{M}~G_{L}U_{F18}~C_{F4}U_{L}~C_{F}U_{F18}~G_{L}G_{F}U_{F}~C_{F4}~C_{F}UU_{L}~A_{F}~C_{L}U_{L}U_{F}~A_{F}~G_{F}
P-U_{F}~U_{M}~G_{L}U_{F12}~C_{F4}U_{L}~C_{F4}U_{F12}~G_{L}G_{F}U_{F}~C_{F4}~C_{F4}UU_{L}~A_{F}~C_{L}U_{L}U_{F}~A_{F}~G_{F}
P-U_{F}~U_{M}~G_{L}U_{F3}~C_{F4}U_{L}~C_{F4}U_{F3}~G_{L}G_{F}U_{F}~C_{F4}~C_{F4}UU_{L}~A_{F}~C_{L}U_{F}U_{F}~A_{F}~G_{F}
P-U_{F}~U_{M}~G_{L}U_{F18}~C_{F4}U_{L}~C_{F4}U_{F18}~G_{L}G_{F}U_{F}~C_{F4}~C_{F4}UU_{L}~A_{F}~C_{L}U_{F}U_{F}~A_{F}~G_{F}
P-U_{F}~U_{M}~G_{L}U_{F12}~C_{F}U_{L}~C_{F}U_{F12}~G_{L}G_{F}U_{F12}~C_{F}~C_{F}UU_{L}~A_{F}~C_{L}U_{F}U_{F}~A_{F}~G_{F}
P-U_{F}~U_{M}~G_{L}U_{F3}~C_{F}U_{L}~C_{F}U_{F3}~G_{L}G_{F}U_{F3}~C_{F}~C_{F}UU_{L}~A_{F}~C_{L}U_{F}U_{F}~A_{F}~G_{F}
P-U_{F}~U_{M}~G_{L}U_{F18}~C_{F}U_{L}~C_{F}U_{F18}~G_{L}G_{F}U_{F18}~C_{F}~C_{F}UU_{L}~A_{F}~C_{L}U_{F}U_{F}~A_{F}~G_{F}
P-U_{F}~U_{M}~G_{L}U_{F12}~C_{F}U_{L}~C_{F}U_{F12}~G_{L}G_{F}U_{F12}~C_{F}~C_{F}UU_{L}~A_{F}~C_{L}U_{F}U_{F}~A_{F}~G_{F}
P-U_{F}~U_{M}~G_{L}U_{F3}~C_{F}U_{L}~C_{F}U_{F3}~G_{L}G_{F}U_{F3}~C_{F}~C_{F}UU_{L}~A_{F}~C_{L}U_{F}U_{F}~A_{F}~G_{F}
P-U_{F}~U_{M}~G_{L}U_{F18}~C_{F}U_{L}~C_{F}U_{F18}~G_{L}G_{F}U_{F18}~C_{F}~C_{F}UU_{L}~A_{F}~C_{L}U_{F}U_{F}~A_{F}~G_{F}
P-U_{F}~U_{M}~G_{L}U_{F12}~C_{F}U_{L}~C_{F}U_{F12}~G_{L}G_{F}U_{F12}~C_{F}~C_{F}UU_{L}~A_{F}~C_{L}U_{F12}U_{F}~A_{F}~G_{F}
P-U_{F}~U_{M}~G_{L}U_{F3}~C_{F}U_{L}~C_{F}U_{F3}~G_{L}G_{F}U_{F3}~C_{F}~C_{F}UU_{L}~A_{F}~C_{L}U_{F3}U_{F}~A_{F}~G_{F}
P-U_{F}~U_{M}~G_{L}U_{F18}~C_{F}U_{L}~C_{F}U_{F18}~G_{L}G_{F}U_{F18}~C_{F}~C_{F}UU_{L}~A_{F}~C_{L}U_{F18}U_{F}~A_{F}~G_{F}
P-U_{F}~U_{M}~G_{L}U_{F12}~C_{F}U_{L}~C_{F4}U_{F12}~G_{L}G_{F}U_{F12}~C_{F4}~C_{F}UU_{L}~A_{F}~C_{L}U_{F}U_{F}~A_{F}~G_{F}
P-U_{F}~U_{M}~G_{L}U_{F3}~C_{F}U_{L}~C_{F4}U_{F3}~G_{L}G_{F}U_{F3}~C_{F4}~C_{F}UU_{L}~A_{F}~C_{L}U_{F}U_{F}~A_{F}~G_{F}
P-U_{F}~U_{M}~G_{L}U_{F18}~C_{F}U_{L}~C_{F4}U_{F18}~G_{L}G_{F}U_{F18}~C_{F4}~C_{F}UU_{L}~A_{F}~C_{L}U_{F}U_{F}~A_{F}~G_{F}
P-U_{F}~U_{M}~G_{L}U_{F12}~C_{F}U_{L}~C_{F4}U_{F12}~G_{L}G_{F}U_{F12}~C_{F4}~C_{F}UU_{L}~A_{F}~C_{L}U_{F}U_{F}~A_{F}~G_{F}
P-U_{F}~U_{M}~G_{L}U_{F3}~C_{F}U_{L}~C_{F4}U_{F3}~G_{L}G_{F}U_{F3}~C_{F4}~C_{F}UU_{L}~A_{F}~C_{L}U_{F}U_{F}~A_{F}~G_{F}
P-U_{F}~U_{M}~G_{L}U_{F18}~C_{F}U_{L}~C_{F4}U_{F18}~G_{L}G_{F}U_{F18}~C_{F4}~C_{F}UU_{L}~A_{F}~C_{L}U_{F}U_{F}~A_{F}~G_{F}
P-U_{F}~U_{M}~G_{L}U_{F12}~C_{F}U_{L}~C_{F4}U_{F12}~G_{L}G_{F}U_{F12}~C_{F4}~C_{F}UU_{L}~A_{F}~C_{L}U_{F12}U_{F}~A_{F}~G_{F}
P-U_{F}~U_{M}~G_{L}U_{F3}~C_{F}U_{L}~C_{F4}U_{F3}~G_{L}G_{F}U_{F3}~C_{F4}~C_{F}UU_{L}~A_{F}~C_{L}U_{F3}U_{F}~A_{F}~G_{F}
P-U_{F}~U_{M}~G_{L}U_{F18}~C_{F}U_{L}~C_{F4}U_{F18}~G_{L}G_{F}U_{F18}~C_{F4}~C_{F}UU_{L}~A_{F}~C_{L}U_{F18}U_{F}~A_{F}~G_{F}
P-U_{F}~U_{M}~G_{L}U_{F12}~C_{F4}U_{L}~C_{F4}U_{F12}~G_{L}G_{F}U_{F12}~C_{F4}~C_{F4}UU_{L}~A_{F}~C_{L}U_{F}U_{F}~A_{F}~G_{F}
P-U_{F}~U_{M}~G_{L}U_{F3}~C_{F4}U_{L}~C_{F4}U_{F3}~G_{L}G_{F}U_{F3}~C_{F4}~C_{F4}UU_{L}~A_{F}~C_{L}U_{F}U_{F}~A_{F}~G_{F}
P-U_{F}~U_{M}~G_{L}U_{F18}~C_{F4}U_{L}~C_{F4}U_{F18}~G_{L}G_{F}U_{F18}~C_{F4}~C_{F4}UU_{L}~A_{F}~C_{L}U_{F}U_{F}~A_{F}~G_{F}
P-U_{F}~U_{M}~G_{L}U_{F12}~C_{F4}U_{L}~C_{F4}U_{F12}~G_{L}G_{F}U_{F12}~C_{F4}~C_{F4}UU_{L}~A_{F}~C_{L}U_{F}U_{F}~A_{F}~G_{F}
P-U_{F}~U_{M}~G_{L}U_{F3}~C_{F4}U_{L}~C_{F4}U_{F3}~G_{L}G_{F}U_{F3}~C_{F4}~C_{F4}UU_{L}~A_{F}~C_{L}U_{F}U_{F}~A_{F}~G_{F}
P-U_{F}~U_{M}~G_{L}U_{F18}~C_{F4}U_{L}~C_{F4}U_{F18}~G_{L}G_{F}U_{F18}~C_{F4}~C_{F4}UU_{L}~A_{F}~C_{L}U_{F}U_{F}~A_{F}~G_{F}
P-U_{F}~U_{M}~G_{L}U_{F12}~C_{F4}U_{L}~C_{F4}U_{F12}~G_{L}G_{F}U_{F12}~C_{F4}~C_{F4}UU_{L}~A_{F}~C_{L}U_{F12}U_{F}~A_{F}~G_{F}
P-U_{F}~U_{M}~G_{L}U_{F3}~C_{F4}U_{L}~C_{F4}U_{F3}~G_{L}G_{F}U_{F3}~C_{F4}~C_{F4}UU_{L}~A_{F}~C_{L}U_{F3}U_{F}~A_{F}~G_{F}
P-U_{F}~U_{M}~G_{L}U_{F18}~C_{F4}U_{L}~C_{F4}U_{F18}~G_{L}G_{F}U_{F18}~C_{F4}~C_{F4}UU_{L}~A_{F}~C_{L4}U_{F18}U_{F}~A_{F}~G_{F}
P-U_{F}~U_{M}~G_{L}U_{F}~C_{F4}U_{L}~C_{F4}A_{F}~G_{L}G_{F}U_{F}~C_{F4}~C_{F4}UU_{L}~A_{F}~C_{L}U_{F}U_{F}~A_{F}~G_{F}
P-U_{F}~U_{M}~G_{L}U_{F}~C_{F4}U_{L}~C_{F4}U_{F}~A_{L}G_{F}U_{F}~C_{F4}~C_{F4}UU_{L}~A_{F}~C_{L}U_{F}U_{F}~A_{F}~G_{F}
P-U_{F}~U_{M}~G_{L}U_{F}~C_{F}U_{L}~C_{F}U_{F}~G_{F}G_{L}U_{F}~C_{F}~C_{L}UU_{F}~A_{F}~C_{L}U_{F}U_{F}~A_{F}~G_{F}
P-U_{F}~U_{M}~G_{L}U_{F12}~C_{F}U_{L}~C_{F4}U_{F12}~G_{F}G_{L}U_{F}~C_{F}~C_{L}UU_{F12}~A_{F}~C_{L}U_{F}U_{F}~A_{F}~G_{F}
P-U_{F}~U_{M}~G_{L}U_{F3}~C_{F}U_{L}~C_{F4}U_{F3}~G_{F}G_{L}U_{F}~C_{F}~C_{L}UU_{F3}~A_{F}~C_{L}U_{F}U_{F}~A_{F}~G_{F}
P-U_{F}~U_{M}~G_{L}U_{F18}~C_{F}U_{L}~C_{F4}U_{F18}~G_{F}G_{L}U_{F}~C_{F}~C_{L}UU_{F18}~A_{F}~C_{L}U_{F}U_{F}~A_{F}~G_{F}
P-U_{F}~U_{M}~G_{L}U_{F12}~C_{F4}U_{L}~C_{F}U_{F12}~G_{F}G_{L}U_{F}~C_{F}~C_{L}UU_{F12}~A_{F}~C_{L}U_{F}U_{F}~A_{F}~G_{F}
P-U_{F}~U_{M}~G_{L}U_{F3}~C_{F4}U_{L}~C_{F}U_{F3}~G_{F}G_{L}U_{F}~C_{F}~C_{L}UU_{F3}~A_{F}~C_{L}U_{F}U_{F}~A_{F}~G_{F}
P-U_{F}~U_{M}~G_{L}U_{F18}~C_{F4}U_{L}~C_{F}U_{F18}~G_{F}G_{L}U_{F}~C_{F}~C_{L}UU_{F18}~A_{F}~C_{L}U_{F}U_{F}~A_{F}~G_{F}
P-U_{F}~U_{M}~G_{L}U_{F12}~C_{F4}U_{L}~C_{F4}U_{F12}~G_{F}G_{L}U_{F}~C_{F4}~C_{L}UU_{F12}~A_{F}~C_{L}U_{F}U_{F}~A_{F}~G_{F}
P-U_{F}~U_{M}~G_{L}U_{F3}~C_{F4}U_{L}~C_{F4}U_{F3}~G_{F}G_{L}U_{F}~C_{F4}~C_{L}UU_{F3}~A_{F}~C_{L}U_{F}U_{F}~A_{F}~G_{F}
P-U_{F}~U_{M}~G_{L}U_{F18}~C_{F4}U_{L}~C_{F4}U_{F18}~G_{F}G_{L}U_{F}~C_{F4}~C_{L}UU_{F18}~A_{F}~C_{L4}U_{F}U_{F}~A_{F}~G_{F}
P-U_{F}~U_{M}~G_{L}U_{F12}~C_{F}U_{L}~C_{F}U_{F12}~G_{F}G_{L}U_{F12}~C_{F}~C_{L}UU_{F12}~A_{F}~C_{L}U_{F}U_{F}~A_{F}~G_{F}
P-U_{F}~U_{M}~G_{L}U_{F3}~C_{F}U_{L}~C_{F}U_{F3}~G_{F}G_{L}U_{F3}~C_{F}~C_{L}UU_{F3}~A_{F}~C_{L}U_{F}U_{F}~A_{F}~G_{F}
P-U_{F}~U_{M}~G_{L}U_{F18}~C_{F}U_{L}~C_{F}U_{F18}~G_{F}G_{L}U_{F18}~C_{F}~C_{L}UU_{F18}~A_{F}~C_{L}U_{F}U_{F}~A_{F}~G_{F}
P-U_{F}~U_{M}~G_{L}U_{F12}~C_{F}U_{L}~C_{F}U_{F12}~G_{F}G_{L}U_{F12}~C_{F}~C_{L}UU_{F12}~A_{F}~C_{L}U_{F}U_{F}~A_{F}~G_{F}
P-U_{F}~U_{M}~G_{L}U_{F3}~C_{F}U_{L}~C_{F}U_{F3}~G_{F}G_{L}U_{F3}~C_{F}~C_{L}UU_{F3}~A_{F}~C_{L}U_{F}U_{F}~A_{F}~G_{F}
P-U_{F}~U_{M}~G_{L}U_{F18}~C_{F}U_{L}~C_{F}U_{F18}~G_{F}G_{L}U_{F18}~C_{F}~C_{L}UU_{F18}~A_{F}~C_{L}U_{F}U_{F}~A_{F}~G_{F}
P-U_{F}~U_{M}~G_{L}U_{F12}~C_{F}U_{L}~C_{F}U_{F12}~G_{F}G_{L}U_{F12}~C_{F}~C_{L}UU_{F12}~A_{F}~C_{L}U_{F12}U_{F}~A_{F}~G_{F}
P-U_{F}~U_{M}~G_{L}U_{F3}~C_{F}U_{L}~C_{F}U_{F3}~G_{F}G_{L}U_{F3}~C_{F}~C_{L}UU_{F3}~A_{F}~C_{L}U_{F3}U_{F}~A_{F}~G_{F}
P-U_{F}~U_{M}~G_{L}U_{F18}~C_{F}U_{L}~C_{F}U_{F18}~G_{F}G_{L}U_{F18}~C_{F}~C_{L}UU_{F18}~A_{F}~C_{L}U_{F18}U_{F}~A_{F}~G_{F}
P-U_{F}~U_{M}~G_{L}U_{F12}~C_{F}U_{L}~C_{F4}U_{F12}~G_{F}G_{L}U_{F12}~C_{F}~C_{L}UU_{F12}~A_{F}~C_{L}U_{F}U_{F}~A_{F}~G_{F}
P-U_{F}~U_{M}~G_{L}U_{F3}~C_{F}U_{L}~C_{F4}U_{F3}~G_{F}G_{L}U_{F3}~C_{F}~C_{L}UU_{F3}~A_{F}~C_{L}U_{F}U_{F}~A_{F}~G_{F}
P-U_{F}~U_{M}~G_{L}U_{F18}~C_{F}U_{L}~C_{F4}U_{F18}~G_{F}G_{L}U_{F18}~C_{F}~C_{L}UU_{F18}~A_{F}~C_{L}U_{F}U_{F}~A_{F}~G_{F}
P-U_{F}~U_{M}~G_{L}U_{F12}~C_{F}U_{L}~C_{F4}U_{F12}~G_{F}G_{L}U_{F12}~C_{F}~C_{L}UU_{F12}~A_{F}~C_{L}U_{F}U_{F}~A_{F}~G_{F}
P-U_{F}~U_{M}~G_{L}U_{F3}~C_{F}U_{L}~C_{F4}U_{F3}~G_{F}G_{L}U_{F3}~C_{F}~C_{L}UU_{F3}~A_{F}~C_{L}U_{F}U_{F}~A_{F}~G_{F}
P-U_{F}~U_{M}~G_{L}U_{F18}~C_{F}U_{L}~C_{F4}U_{F18}~G_{F}G_{L}U_{F18}~C_{F}~C_{L}UU_{F18}~A_{F}~C_{L}U_{F}U_{F}~A_{F}~G_{F}
P-U_{F}~U_{M}~G_{L}U_{F12}~C_{F}U_{L}~C_{F4}U_{F12}~G_{F}G_{L}U_{F12}~C_{F4}~C_{L}UU_{F12}~A_{F}~C_{L}U_{F12}U_{F}~A_{F}~G_{F}
P-U_{F}~U_{M}~G_{L}U_{F3}~C_{F}U_{L}~C_{F4}U_{F3}~G_{F}G_{L}U_{F3}~C_{F4}~C_{L}UU_{F3}~A_{F}~C_{L}U_{F3}U_{F}~A_{F}~G_{F}
P-U_{F}~U_{M}~G_{L}U_{F18}~C_{F}U_{L}~C_{F4}U_{F18}~G_{F}G_{L}U_{F18}~C_{F4}~C_{L}UU_{F18}~A_{F}~C_{L}U_{F18}U_{F}~A_{F}~G_{F}
P-U_{F}~U_{M}~G_{L}U_{F12}~C_{F4}U_{L}~C_{F4}U_{F12}~G_{F}G_{L}U_{F12}~C_{F}~C_{L}UU_{F12}~A_{F}~C_{L}U_{F}U_{F}~A_{F}~G_{F}
P-U_{F}~U_{M}~G_{L}U_{F3}~C_{F4}U_{L}~C_{F4}U_{F3}~G_{F}G_{L}U_{F3}~C_{F}~C_{L}UU_{F3}~A_{F}~C_{L}U_{F}U_{F}~A_{F}~G_{F}
P-U_{F}~U_{M}~G_{L}U_{F18}~C_{F4}U_{L}~C_{F4}U_{F18}~G_{F}G_{L}U_{F18}~C_{F}~C_{L}UU_{F18}~A_{F}~C_{L}U_{F}U_{F}~A_{F}~G_{F}
P-U_{F}~U_{M}~G_{L}U_{F12}~C_{F4}U_{L}~C_{F4}U_{F12}~G_{F}G_{L}U_{F12}~C_{F}~C_{L}UU_{F12}~A_{F}~C_{L}U_{F}U_{F}~A_{F}~G_{F}
P-U_{F}~U_{M}~G_{L}U_{F3}~C_{F4}U_{L}~C_{F4}U_{F3}~G_{F}G_{L}U_{F3}~C_{F}~C_{L}UU_{F3}~A_{F}~C_{L}U_{F}U_{F}~A_{F}~G_{F}
P-U_{F}~U_{M}~G_{L}U_{F18}~C_{F4}U_{L}~C_{F4}U_{F18}~G_{F}G_{L}U_{F18}~C_{F}~C_{L}UU_{F18}~A_{F}~C_{L}U_{F}U_{F}~A_{F}~G_{F}
P-U_{F}~U_{M}~G_{L}U_{F12}~C_{F4}U_{L}~C_{F4}U_{F12}~G_{F}G_{L}U_{F12}~C_{F4}~C_{L}UU_{F12}~A_{F}~C_{L}U_{F12}U_{F}~A_{F}~G_{F}
P-U_{F}~U_{M}~G_{L}U_{F3}~C_{F4}U_{L}~C_{F4}U_{F3}~G_{F}G_{L}U_{F3}~C_{F4}~C_{L}UU_{F3}~A_{F}~C_{L}U_{F3}U_{F}~A_{F}~G_{F}
P-U_{F}~U_{M}~G_{L}U_{F18}~C_{F4}U_{L}~C_{F4}U_{F18}~G_{F}G_{L}U_{F18}~C_{F4}~C_{L}UU_{F18}~A_{F}~C_{L}U_{F18}U_{F}~A_{F}~G_{F}
P-U_{F}~U_{M}~G_{L}U_{F}~C_{F4}U_{L}~C_{F4}A_{F}~G_{F}G_{L}U_{F}~C_{F}~C_{L}UU_{F}~A_{F}~C_{L}U_{F}U_{F}~A_{F}~G_{F}
P-U_{F}~U_{M}~G_{L}U_{F}~C_{F4}U_{L}~C_{F4}U_{F}~A_{F}G_{L}U_{F}~C_{F}~C_{L}UU_{F}~A_{F}~C_{L}U_{F}U_{F}~A_{F}~G_{F}

TABLE 8B
Compositions of Matter Suitable for Administration to a Subject
with or without a Protective Carrier and for Any Other use as
Provided for herein (5′-3′)

P-U_{M}~U_{F}~G_{M}U_{F12}~C_{M}U_{F}~C_{M4}U_{F12}~G_{M}G_{F}~U_{M}C_{F}~C_{M4}U~U_{M12}~A_{F}~C_{M}~U_{F}~U_{M}~A_{L}~A_{L}~G_{M}
P-U_{M}~U_{F}~G_{M}U_{F3}~C_{M}U_{F}~C_{M4}U_{F3}~G_{M}G_{F}~U_{M}C_{F}~C_{M4}U~U_{M3}~A_{F}~C_{M}~U_{F}~U_{M}~A_{L}~A_{L}~G_{M}
P-U_{M}~U_{F}~G_{M}U_{F18}~C_{M}U_{F}~C_{M4}U_{F18}~G_{M}G_{F}~U_{M}C_{F}~C_{M4}U~U_{M18}~A_{F}~C_{M}~U_{F}~U_{M}~A_{L}~A_{L}~G_{M}
P-U_{M}~U_{F}~G_{M}U_{F12}~C_{M4}U_{F}~C_{M}U_{F12}~G_{M}G_{F}~U_{M}C_{F4}~C_{M}U~U_{M12}~A_{F}~C_{M4}~U_{F}~U_{M}~A_{L}~A_{L}~G_{M}
P-U_{M}~U_{F}~G_{M}U_{F3}~C_{M4}U_{F}~C_{M}U_{F3}~G_{M}G_{F}~U_{M}C_{F4}~C_{M}U~U_{M3}~A_{F}~C_{M4}~U_{F}~U_{M}~A_{L}~A_{L}~G_{M}
P-U_{M}~U_{F}~G_{M}U_{F18}~C_{M4}U_{F}~C_{M}U_{F18}~G_{M}G_{F}~U_{M}C_{F4}~C_{M}U~U_{M18}~A_{F}~C_{M4}~U_{F}~U_{M}~A_{L}~A_{L}~G_{M}
P-U_{M}~U_{F}~G_{M}U_{F12}~C_{M4}U_{F}~C_{M4}U_{F12}~G_{M}G_{F}~U_{M}C_{F4}~C_{M4}U~U_{M12}~A_{F}~C_{M4}~U_{F}~U_{M}~A_{L}~A_{L}~G_{M}
P-U_{M}~U_{F}~G_{M}U_{F3}~C_{M4}U_{F}~C_{M4}U_{F3}~G_{M}G_{F}~U_{M}C_{F4}~C_{M4}U~U_{M3}~A_{F}~C_{M4}~U_{F}~U_{M}~A_{L}~A_{L}~G_{M}
P-U_{M}~U_{F}~G_{M}U_{F18}~C_{M4}U_{F}~C_{M4}U_{F18}~G_{M}G_{F}~U_{M}C_{F4}~C_{M4}U~U_{M18}~A_{F}~C_{M4}~U_{F}~U_{M}~A_{L}~A_{L}~G_{M}
P-U_{M}~U_{F}~G_{M}U_{F12}~C_{M}U_{F}~C_{M}U_{F12}~G_{M}G_{F}~U_{M12}C_{F}~C_{M}U~U_{M12}~A_{F}~C_{M}~U_{F}~U_{M}~A_{L}~A_{L}~G_{M}
P-U_{M}~U_{F}~G_{M}U_{F3}~C_{M}U_{F}~C_{M}U_{F3}~G_{M}G_{F}~U_{M3}C_{F}~C_{M}U~U_{M3}~A_{F}~C_{M}~U_{F}~U_{M}~A_{L}~A_{L}~G_{M}
P-U_{M}~U_{F}~G_{M}U_{F18}~C_{M}U_{F}~C_{M}U_{F18}~G_{M}G_{F}~U_{M18}C_{F}~C_{M}U~U_{M18}~A_{F}~C_{M}~U_{F}~U_{M}~A_{L}~A_{L}~G_{M}
P-U_{M}~U_{F}~G_{M}U_{F12}~C_{M}U_{F12}~C_{M}U_{F12}~G_{M}G_{F}~U_{M12}C_{F}~C_{M}U~U_{M12}~A_{F}~C_{M}~U_{F}~U_{M}~A_{L}~A_{L}~G_{M}
P-U_{M}~U_{F}~G_{M}U_{F3}~C_{M}U_{F3}~C_{M}U_{F3}~G_{M}G_{F}~U_{M3}C_{F}~C_{M}U~U_{M3}~A_{F}~C_{M}~U_{F}~U_{M}~A_{L}~A_{L}~G_{M}
P-U_{M}~U_{F}~G_{M}U_{F18}~C_{M}U_{F18}~C_{M}U_{F18}~G_{M}G_{F}~U_{M18}C_{F}~C_{M}U~U_{M18}~A_{F}~C_{M}~U_{F}~U_{M}~A_{L}~A_{L}~G_{M}
P-U_{M}~U_{F}~G_{M}U_{F12}~C_{M}U_{F12}~C_{M}U_{F12}~G_{M}G_{F}~U_{M12}C_{F}~C_{M}U~U_{M12}~A_{F}~C_{M}~U_{F12}~U_{M}~A_{L}~A_{L}~G_{M}
P-U_{M}~U_{F}~G_{M}U_{F3}~C_{M}U_{F3}~C_{M4}U_{F3}~G_{M}G_{F}~U_{M3}C_{F4}~C_{M}U~U_{M3}~A_{F}~C_{M}~U_{F3}~U_{M}~A_{L}~A_{L}~G_{M}
P-U_{M}~U_{F}~G_{M}U_{F18}~C_{M}U_{F18}~C_{M4}U_{F18}~G_{M}G_{F}~U_{M18}C_{F}~C_{M}U~U_{M18}~A_{F}~C_{M}~U_{F18}~U_{M}~A_{L}~A_{L}~G_{M}
P-U_{M}~U_{F}~G_{M}U_{F12}~C_{M}U_{F}~C_{M4}U_{F12}~G_{M}G_{F}~U_{M12}C_{F4}~C_{M}U~U_{M12}~A_{F}~C_{M4}~U_{F}~U_{M}~A_{L}~A_{L}~G_{M}
P-U_{M}~U_{F}~G_{M}U_{F3}~C_{M}U_{F}~C_{M4}U_{F3}~G_{M}G_{F}~U_{M3}C_{F4}~C_{M}U~U_{M3}~A_{F}~C_{M4}~U_{F}~U_{M}~A_{L}~A_{L}~G_{M}
P-U_{M}~U_{F}~G_{M}U_{F18}~C_{M}U_{F}~C_{M4}U_{F18}~G_{M}G_{F}~U_{M18}C_{F4}~C_{M}U~U_{M18}~A_{F}~C_{M4}~U_{F}~U_{M}~A_{L}~A_{L}~G_{M}
P-U_{M}~U_{F}~G_{M}U_{F12}~C_{M}U_{F12}~C_{M4}U_{F12}~G_{M}G_{F}~U_{M12}C_{F4}~C_{M}U~U_{M12}~A_{F}~C_{M4}~U_{F}~U_{M}~A_{L}~A_{L}~G_{M}
P-U_{M}~U_{F}~G_{M}U_{F3}~C_{M}U_{L}F3~C_{M4}U_{F3}~G_{M}G_{F}~U_{M3}C_{F4}~C_{M}U~U_{M3}~A_{F}~C_{M4}~U_{F}~U_{M}~A_{L}~A_{L}~G_{M}
P-U_{M}~U_{F}~G_{M}U_{F18}~C_{M}U_{F18}~C_{M4}U_{F18}~G_{M}G_{F}~U_{M18}C_{F4}~C_{M}U~U_{M18}~A_{F}~C_{M4}~U_{F}~U_{M}~A_{L}~A_{L}~G_{M}
P-U_{M}~U_{F}~G_{M}U_{F12}~C_{M}U_{F12}~C_{M4}U_{F12}~G_{M}G_{F}~U_{M12}C_{F4}~C_{M}U~U_{M12}~A_{F}~C_{M4}~U_{F12}~U_{M}~A_{L}~A_{L}~G_{M}
P-U_{M}~U_{F}~G_{M}U_{F3}~C_{M}U_{F3}~C_{M4}U_{F3}~G_{M}G_{F}~U_{M3}C_{F4}~C_{M}U~U_{M3}~A_{F}~C_{M4}~U_{F3}~U_{M}~A_{L}~A_{L}~G_{M}
P-U_{M}~U_{F}~G_{M}U_{F18}~C_{M}U_{F18}~C_{M4}U_{F18}~G_{M}G_{F}~U_{M18}C_{F4}~C_{M}U~U_{M18}~A_{F}~C_{M4}~U_{F18}~U_{M}~A_{L}~A_{L}~G_{M}
P-U_{M}~U_{F}~G_{M}U_{F12}~C_{M4}U_{F}~C_{M4}U_{F12}~G_{M}G_{F}~U_{M12}C_{F4}~C_{M4}U~U_{M12}~A_{F}~C_{M4}~U_{F}~U_{M}~A_{L}~A_{L}~G_{M}
P-U_{M}~U_{F}~G_{M}U_{F3}~C_{M4}U_{F}~C_{M4}U_{F3}~G_{M}G_{F}~U_{M3}C_{F4}~C_{M4}U~U_{M3}~A_{F}~C_{M4}~U_{F}~U_{M}~A_{L}~A_{L}~G_{M}
P-U_{M}~U_{F}~G_{M}U_{F18}~C_{M4}U_{F}~C_{M4}U_{F18}~G_{M}G_{F}~U_{M18}C_{F4}~C_{M4}U~U_{M18}~A_{F}~C_{M4}~U_{F}~U_{M}~A_{L}~A_{L}~G_{M}
P-U_{M}~U_{F}~G_{M}U_{F12}~C_{M4}U_{F12}~C_{M4}U_{F12}~G_{M}G_{F}~U_{M12}C_{F4}~C_{M4}U~U_{M12}~A_{F}~C_{M4}~U_{F}~U_{M}~A_{L}~A_{L}~G_{M}
P-U_{M}~U_{F}~G_{M}U_{F3}~C_{M4}U_{F3}~C_{M4}U_{F3}~G_{M}G_{F}~U_{M3}C_{F4}~C_{M4}U~U_{M3}~A_{F}~C_{M4}~U_{F}~U_{M}~A_{L}~A_{L}~G_{M}
P-U_{M}~U_{F}~G_{M}U_{F18}~C_{M4}U_{F18}~C_{M4}U_{F18}~G_{M}G_{F}~U_{M18}C_{F4}~C_{M4}U~U_{M18}~A_{F}~C_{M4}~U_{F}~U_{M}~A_{L}~A_{L}~G_{M}
P-U_{M}~U_{F}~G_{M}U_{F12}~C_{M4}U_{F12}~C_{M4}U_{F12}~G_{M}G_{F}~U_{M12}C_{F4}~C_{M4}U~U_{M12}~A_{F}~C_{M4}~U_{F12}~U_{M}~A_{L}~A_{L}~G_{M}
P-U_{M}~U_{F}~G_{M}U_{F3}~C_{M4}U_{F3}~C_{M4}U_{F3}~G_{M}G_{F}~U_{M3}C_{F4}~C_{M4}U~U_{M3}~A_{F}~C_{M4}~U_{F3}~U_{M}~A_{L}~A_{L}~G_{M}
P-U_{M}~U_{F}~G_{M}U_{F18}~C_{M4}U_{F18}~C_{M4}U_{F18}~G_{M}G_{F}~U_{M18}C_{F4}~C_{M4}U~U_{M18}~A_{F}~C_{M4}~U_{F18}~U_{M}~A_{L}~A_{L}~G_{M}
P-U_{M}~U_{F}~G_{M}U_{F}~C_{M4}U_{F}~C_{M4}A_{F}~G_{M}G_{F}~U_{M}C_{F4}~C_{M4}U~U_{M}~A_{F}~C_{M4}~U_{F}~U_{M}~A_{L}~A_{L}~G_{M}
P-U_{M}~U_{F}~G_{M}U_{F}~C_{M4}U_{F}~C_{M4}A_{F}~A_{M}G_{F}~U_{M}C_{F4}~C_{M4}U~U_{M}~A_{F}~C_{M4}~U_{F}~U_{M}~A_{L}~A_{L}~G_{M}
P-U_{M}~U_{F}~G_{L}U_{F}~C_{M}U_{L}~C_{M}U_{F}~G_{L}G_{F}~U_{M}C_{F}~C_{L}U~U_{M}~A_{F}~C_{L}~U_{F}~U_{M}~A_{L}~A_{L}~G_{M}
P-U_{M}~U_{F}~G_{L}U_{F12}~C_{M}U_{L}~C_{M4}U_{F12}~G_{L}G_{F}~U_{M}C_{F}~C_{L4}U~U_{M12}~A_{F}~C_{L}~U_{F}~U_{M}~A_{L}~A_{L}~G_{M}
P-U_{M}~U_{F}~G_{L}U_{F3}~C_{M}U_{L}~C_{M4}U_{F3}~G_{L}G_{F}~U_{M}C_{F}~C_{L4}U~U_{M3}~A_{F}~C_{L}~U_{F}~U_{M}~A_{L}~A_{L}~G_{M}
P-U_{M}~U_{F}~G_{L}U_{F18}~C_{M}U_{L}~C_{M4}U_{F18}~G_{L}G_{F}~U_{M}C_{F}~C_{L4}U~U_{M18}~A_{F}~C_{L}~U_{F}~U_{M}~A_{L}~A_{L}~G_{M}
P-U_{M}~U_{F}~G_{L}U_{F12}~C_{M4}U_{L}~C_{M}U_{F12}~G_{L}G_{F}~U_{M}C_{F4}~C_{L}U~U_{M12}~A_{F}~C_{L4}~U_{F}~U_{M}~A_{L}~A_{L}~G_{M}
P-U_{M}~U_{F}~G_{L}U_{F3}~C_{M4}U_{L}~C_{M}U_{F3}~G_{L}G_{F}~U_{M}C_{F4}~C_{L}U~U_{M3}~A_{F}~C_{L4}~U_{F}~U_{M}~A_{L}~A_{L}~G_{M}
P-U_{M}~U_{F}~G_{L}U_{F18}~C_{M4}U_{L}~C_{M}U_{F18}~G_{L}G_{F}~U_{M}C_{F4}~C_{L}U~U_{M18}~A_{F}~C_{L4}~U_{F}~U_{M}~A_{L}~A_{L}~G_{M}
P-U_{M}~U_{F}~G_{L}U_{F12}~C_{M4}U_{L}~C_{M4}U_{F12}~G_{L}G_{F}~U_{M}C_{F4}~C_{L4}U~U_{M12}~A_{F}~C_{L4}~U_{F}~U_{M}~A_{L}~A_{L}~G_{M}
P-U_{M}~U_{F}~G_{L}U_{F3}~C_{M4}U_{L}~C_{M4}U_{F3}~G_{L}G_{F}~U_{M}C_{F4}~C_{L4}U~U_{M3}~A_{F}~C_{L4}~U_{F}~U_{M}~A_{L}~A_{L}~G_{M}
P-U_{M}~U_{F}~G_{L}U_{F18}~C_{M4}U_{L}~C_{M4}U_{F18}~G_{L}G_{F}~U_{M}C_{F4}~C_{L4}U~U_{M18}~A_{F}~C_{L4}~U_{F}~U_{M}~A_{L}~A_{L}~G_{M}
P-U_{M}~U_{F}~G_{L}U_{F12}~C_{M}U_{L}~C_{M}U_{F12}~G_{L}G_{F}~U_{M12}C_{F}~C_{L}U~U_{M12}~A_{F}~C_{L}~U_{F}~U_{M}~A_{L}~A_{L}~G_{M}
P-U_{M}~U_{F}~G_{L}U_{F3}~C_{M}U_{L}~C_{M}U_{F3}~G_{L}G_{F}~U_{M3}C_{F}~C_{L}U~U_{M3}~A_{F}~C_{L}~U_{F}~U_{M}~A_{L}~A_{L}~G_{M}
P-U_{M}~U_{F}~G_{L}U_{F18}~C_{M}U_{L}~C_{M}U_{F18}~G_{L}G_{F}~U_{M18}C_{F}~C_{L}U~U_{M18}~A_{F}~C_{L}~U_{F}~U_{M}~A_{L}~A_{L}~G_{M}
P-U_{M}~U_{F}~G_{L}U_{F12}~C_{M}U_{L12}~C_{M}U_{F12}~G_{L}G_{F}~U_{M12}C_{F}~C_{L}U~U_{M12}~A_{F}~C_{L}~U_{F}~U_{M}~A_{L}~A_{L}~G_{M}
P-U_{M}~U_{F}~G_{L}U_{F3}~C_{M}U_{L3}~C_{M}U_{F3}~G_{L}G_{F}~U_{M3}C_{F}~C_{L}U~U_{M3}~A_{F}~C_{L}~U_{F}~U_{M}~A_{L}~A_{L}~G_{M}
P-U_{M}~U_{F}~G_{L}U_{F18}~C_{M}U_{L18}~C_{M}U_{F18}~G_{L}G_{F}~U_{M18}C_{F}~C_{L}U~U_{M18}~A_{F}~C_{L}~U_{F}~U_{M}~A_{L}~A_{L}~G_{M}
P-U_{M}~U_{F}~G_{L}U_{F12}~C_{M}U_{L12}~C_{M}U_{F12}~G_{L}G_{F}~U_{M12}C_{F}~C_{L}U~U_{M12}~AL~C_{L}~U_{F12}~U_{M}~A_{L}~A_{L}~G_{M}
P-U_{M}~U_{F}~G_{L}U_{F3}~C_{M}U_{L3}~C_{M}U_{F3}~G_{L}G_{F}~U_{M3}C_{F}~C_{L}U~U_{M3}~A_{F}~C_{L}~U_{F3}~U_{M}~A_{L}~A_{L}~G_{M}
P-U_{M}~U_{F}~G_{L}U_{F18}~C_{M}U_{L18}~C_{M}U_{F18}~G_{L}G_{F}~U_{M18}C_{F}~C_{L}U~U_{M18}~A_{F}~C_{L}~U_{F18}~U_{M}~A_{L}~A_{L}~G_{M}
P-U_{M}~U_{F}~G_{L}U_{F12}~C_{M}U_{L}~C_{M4}U_{F12}~G_{L}G_{F}~U_{M12}C_{F4}~C_{L}U~U_{M12}~A_{F}~C_{L4}~U_{F}~U_{M}~A_{L}~A_{L}~G_{M}
P-U_{M}~U_{F}~G_{L}U_{F3}~C_{M}U_{L}~C_{M4}U_{F3}~G_{L}G_{F}~U_{M3}C_{F4}~C_{L}U~U_{M3}~A_{F}~C_{L4}~U_{F}~U_{M}~A_{L}~A_{L}~G_{M}
P-U_{M}~U_{F}~G_{L}U_{F18}~C_{M}U_{L}~C_{M4}U_{F18}~G_{L}G_{F}~U_{M18}C_{F4}~C_{L}U~U_{M18}~A_{F}~C_{L4}~U_{F}~U_{M}~A_{L}~A_{L}~G_{M}
P-U_{M}~U_{F}~G_{L}U_{F12}~C_{M}U_{L12}~C_{M4}U_{F12}~G_{L}G_{F}~U_{M12}C_{F4}~C_{L}U~U_{M12}~A_{F}~C_{L4}~U_{F}~U_{M}~A_{L}~A_{L}~G_{M}
P-U_{M}~U_{F}~G_{L}U_{F3}~C_{M}U_{L3}~C_{M4}U_{F3}~G_{L}G_{F}~U_{M3}C_{F4}~C_{L}U~U_{M3}~A_{F}~C_{L4}~U_{F}~U_{M}~A_{L}~A_{L}~G_{M}
P-U_{M}~U_{F}~G_{L}U_{F18}~C_{M}U_{L18}~C_{M4}U_{F18}~G_{L}G_{F}~U_{M18}C_{F4}~C_{L}U~U_{M18}~A_{F}~C_{L4}~U_{F}~U_{M}~A_{L}~A_{L}~G_{M}
P-U_{M}~U_{F}~G_{L}U_{F12}~C_{M}U_{L12}~C_{M4}U_{F12}~G_{L}G_{F}~U_{M12}C_{F4}~C_{L}U~U_{M12}~A_{F}~C_{L4}~U_{F12}~U_{M}~A_{L}~A_{L}~G_{M}
P-U_{M}~U_{F}~G_{L}U_{F3}~C_{M}U_{L3}~C_{M4}U_{F3}~G_{L}G_{F}~U_{M3}C_{F4}~C_{L}U~U_{M3}~A_{F}~C_{L4}~U_{F3}~U_{M}~A_{L}~A_{L}~G_{M}
P-U_{M}~U_{F}~G_{L}U_{F18}~C_{M}U_{L18}~C_{M4}U_{F18}~G_{L}G_{F}~U_{M18}C_{F4}~C_{L}U~U_{M18}~A_{F}~C_{L4}~U_{F18}~U_{M}~A_{L}~A_{L}~G_{M}
P-U_{M}~U_{F}~G_{L}U_{F12}~C_{M4}U_{L}~C_{M4}U_{F12}~G_{L}G_{F}~U_{M12}C_{F4}~C_{L4}U~U_{M12}~A_{F}~C_{L4}~U_{F}~U_{M}~A_{L}~A_{L}~G_{M}
P-U_{M}~U_{F}~G_{L}U_{F3}~C_{M4}U_{L}~C_{M4}U_{F3}~G_{L}G_{F}~U_{M3}C_{F4}~C_{L4}U~U_{M3}~A_{F}~C_{L4}~U_{F}~U_{M}~A_{L}~A_{L}~G_{M}
P-U_{M}~U_{F}~G_{L}U_{F18}~C_{M4}U_{L}~C_{M4}U_{F18}~G_{L}G_{F}~U_{M18}C_{F4}~C_{L4}U~U_{M18}~A_{F}~C_{L4}~U_{F}~U_{M}~A_{L}~A_{L}~G_{M}
P-U_{M}~U_{F}~G_{L}U_{F12}~C_{M4}U_{L12}~C_{M4}U_{F12}~G_{L}G_{F}~U_{M12}C_{F4}~C_{L4}U~U_{M12}~A_{F}~C_{L4}~U_{F}~U_{M}~A_{L}~A_{L}~G_{M}
P-U_{M}~U_{F}~G_{L}U_{F3}~C_{M4}U_{L3}~C_{M4}U_{F3}~G_{L}G_{F}~U_{M3}C_{F4}~C_{L4}U~U_{M3}~A_{F}~C_{L4}~U_{F}~U_{M}~A_{L}~A_{L}~G_{M}
P-U_{M}~U_{F}~G_{L}U_{F18}~C_{M4}U_{L18}~C_{M4}U_{F18}~G_{L}G_{F}~U_{M18}C_{F4}~C_{L4}U~U_{M18}~A_{F}~C_{L4}~U_{F}~U_{M}~A_{L}~A_{L}~G_{M}
P-U_{M}~U_{F}~G_{L}U_{F12}~C_{M4}U_{L12}~C_{M4}U_{F12}~G_{L}G_{F}~U_{M12}C_{F4}~C_{L4}U~U_{M12}~A_{F}~C_{L4}~U_{F12}~U_{M}~A_{L}~A_{L}~G_{M}
P-U_{M}~U_{F}~G_{L}U_{F3}~C_{M4}U_{L3}~C_{M4}U_{F3}~G_{L}G_{F}~U_{M3}C_{F4}~C_{L4}U~U_{M3}~A_{F}~C_{L4}~U_{F3}~U_{M}~A_{L}~A_{L}~G_{M}
P-U_{M}~U_{F}~G_{L}U_{F18}~C_{M4}U_{L18}~C_{M4}U_{F18}~G_{L}G_{F}~U_{M18}C_{F4}~C_{L4}U~U_{M18}~A_{F}~C_{L4}~U_{F18}~U_{M}~A_{L}~A_{L}~G_{M}
P-U_{M}~U_{F}~G_{L}U_{F}~C_{M4}U_{L}~C_{M4}A_{F}~G_{L}G_{F}~U_{M}C_{F4}~C_{L4}U~U_{M}~A_{F}~C_{L4}~U_{F}~U_{M}~A_{L}~A_{L}~G_{M}
P-U_{M}~U_{F}~G_{L}U_{F}~C_{M4}U_{L}~C_{M4}U_{F}~A_{L}G_{F}~U_{M}C_{F4}~C_{L4}U~U_{M}~A_{F}~C_{L4}~U_{F}~U_{M}~A_{L}~A_{L}~G_{M}
P-U_{M}~U_{F}~G_{M}U_{L}~C_{M}U_{F}~C_{M}U_{L}~G_{M}G_{F}~U_{M}C_{L}~C_{M}U~U_{L}~A_{F}~C_{M}~U_{L}~U_{M}~A_{L}~A_{L}~G_{M}
P-U_{M}~U_{F}~G_{M}U_{L12}~C_{M}U_{F}~C_{M4}U_{L12}~G_{M}G_{F}~U_{M}C_{L}~C_{M4}U~U_{L12}~A_{F}~C_{M}~U_{L}~U_{M}~A_{L}~A_{L}~G_{M}
P-U_{M}~U_{F}~G_{M}U_{L3}~C_{M}U_{F}~C_{M4}U_{L3}~G_{M}G_{F}~U_{M}C_{L}~C_{M4}U~U_{L3}~A_{F}~C_{M}~U_{L}~U_{M}~A_{L}~A_{L}~G_{M}
P-U_{M}~U_{F}~G_{M}U_{L18}~C_{M}U_{F}~C_{M4}U_{L18}~G_{M}G_{F}~U_{M}C_{L}~C_{M4}U~U_{L18}~A_{F}~C_{M}~U_{L}~U_{M}~A_{L}~A_{L}~G_{M}
P-U_{M}~U_{F}~G_{M}U_{L12}~C_{M4}U_{F}~C_{M}U_{L12}~G_{M}G_{F}~U_{M}C_{L4}~C_{M}U~U_{L12}~A_{F}~C_{M4}~U_{L}~U_{M}~A_{L}~A_{L}~G_{M}
P-U_{M}~U_{F}~G_{M}U_{L3}~C_{M4}U_{F}~C_{M}U_{L3}~G_{M}G_{F}~U_{M}C_{L4}~C_{M}U~U_{L3}~A_{F}~C_{M4}~U_{L}~U_{M}~A_{L}~A_{L}~G_{M}
P-U_{M}~U_{F}~G_{M}U_{L18}~C_{M4}U_{F}~C_{M}U_{L18}~G_{M}G_{F}~U_{M}C_{L4}~C_{M}U~U_{L18}~A_{F}~C_{M4}~U_{L}~U_{M}~A_{L}~A_{L}~G_{M}
P-U_{M}~U_{F}~G_{M}U_{L12}~C_{M4}U_{F}~C_{M4}U_{L12}~G_{M}G_{F}~U_{M}C_{L4}~C_{M4}U~U_{L12}~A_{F}~C_{M4}~U_{L}~U_{M}~A_{L}~A_{L}~G_{M}
P-U_{M}~U_{F}~G_{M}U_{L3}~C_{M4}U_{F}~C_{M4}U_{L3}~G_{M}G_{F}~U_{M}C_{L4}~C_{M4}U~U_{L3}~A_{F}~C_{M4}~U_{L}~U_{M}~A_{L}~A_{L}~G_{M}
P-U_{M}~U_{F}~G_{M}U_{L18}~C_{M4}U_{F}~C_{M4}U_{L18}~G_{M}G_{F}~U_{M}C_{L4}~C_{M4}U~U_{L18}~A_{F}~C_{M4}~U_{L}~U_{M}~A_{L}~A_{L}~G_{M}
P-U_{M}~U_{F}~G_{M}U_{L12}~C_{M}U_{F}~C_{M}U_{L12}~G_{M}G_{F}~U_{M12}C_{L}~C_{M}U~U_{L12}~A_{F}~C_{M}~U_{L}~U_{M}~A_{L}~A_{L}~G_{M}
P-U_{M}~U_{F}~G_{M}U_{L3}~C_{M}U_{F}~C_{M}U_{L3}~G_{M}G_{F}~U_{M3}C_{L}~C_{M}U~U_{L3}~A_{F}~C_{M}~U_{L}~U_{M}~A_{L}~A_{L}~G_{M}
P-U_{M}~U_{F}~G_{M}U_{L18}~C_{M}U_{F}~C_{M}U_{L18}~G_{M}G_{F}~U_{M18}C_{L}~C_{M}U~U_{L18}~A_{F}~C_{M}~U_{L}~U_{M}~A_{L}~A_{L}~G_{M}
P-U_{M}~U_{F}~G_{M}U_{L12}~C_{M}U_{F12}~C_{M}U_{L12}~G_{M}G_{F}~U_{M12}C_{L}~C_{M}U~U_{L12}~A_{F}~C_{M}~U_{L}~U_{M}~A_{L}~A_{L}~G_{M}
P-U_{M}~U_{F}~G_{M}U_{L3}~C_{M}U_{F3}~C_{M}U_{L3}~G_{M}G_{F}~U_{M3}C_{L}~C_{M}U~U_{L3}~A_{F}~C_{M}~U_{L}~U_{M}~A_{L}~A_{L}~G_{M}
P-U_{M}~U_{F}~G_{M}U_{L18}~C_{M}U_{F18}~C_{M}U_{L18}~G_{M}G_{F}~U_{M1S}C_{L}~C_{M}U~U_{L18}~A_{F}~C_{M}~U_{L}~U_{M}~A_{L}~A_{L}~G_{M}
P-U_{M}~U_{F}~G_{M}U_{F12}~C_{M}U_{F12}~C_{M}U_{L12}~G_{M}G_{F}~U_{M12}C_{L}~C_{M}U~U_{L12}~A_{F}~C_{M}~U_{L12}~U_{M}~A_{L}~A_{L}~G_{M}
P-U_{M}~U_{F}~G_{M}U_{L3}~C_{M}U_{F3}~C_{M}U_{L3}~G_{M}G_{F}~U_{M3}C_{L}~C_{M}U~U_{L3}~A_{F}~C_{M}~U_{M}~A_{L}~A_{L}~G_{M}
P-U_{M}~U_{F}~G_{M}U_{L18}~C_{M}U_{F18}~C_{M}U_{L18}~G_{M}G_{F}~U_{M1S}C_{L}~C_{M}U~U_{L18}~A_{F}~C_{M}~U_{L18}~U_{M}~A_{L}~A_{L}~G_{M}
P-U_{M}~U_{F}~G_{M}U_{L12}~C_{M}U_{F}~C_{M4}U_{L12}~G_{M}G_{F}~U_{M12}C_{L4}~C_{M}U~U_{L12}~A_{F}~C_{M4}~U_{L}~U_{M}~A_{L}~A_{L}~G_{M}
P-U_{M}~U_{F}~G_{M}U_{L3}~C_{M}U_{F}~C_{M4}U_{L3}~G_{M}G_{F}~U_{M3}C_{L4}~C_{M}U~U_{L3}~A_{F}~C_{M4}~U_{L}~U_{M}~A_{L}~A_{L}~G_{M}
P-U_{M}~U_{F}~G_{M}U_{L18}~C_{M}U_{F}~C_{M4}U_{L18}~G_{M}G_{F}~U_{M18}C_{L4}~C_{M}U~U_{L18}~A_{F}~C_{M4}~U_{L}~U_{M}~A_{L}~A_{L}~G_{M}
P-U_{M}~U_{F}~G_{M}U_{L12}~C_{M}U_{F12}~C_{M4}U_{L12}~G_{M}G_{F}~U_{M12}C_{L4}~C_{M}U~U_{L12}~A_{F}~C_{M4}~U_{L}~U_{M}~A_{L}~A_{L}~G_{M}
P-U_{M}~U_{F}~G_{M}U_{L3}~C_{M}U_{F3}~C_{M4}U_{L3}~G_{M}G_{F}~U_{M3}C_{L4}~C_{M}U~U_{L3}~A_{F}~C_{M4}~U_{L}~U_{M}~A_{L}~A_{L}~G_{M}
P-U_{M}~U_{F}~G_{M}U_{L18}~C_{M}U_{F18}~C_{M4}U_{L18}~G_{M}G_{F}~U_{M18}C_{L4}~C_{M}U~U_{L18}~A_{F}~C_{M4}~U_{L}~U_{M}~A_{L}~A_{L}~G_{M}
P-U_{M}~U_{F}~G_{M}U_{L12}~C_{M}U_{F12}~C_{M4}U_{L12}~G_{M}G_{F}~U_{M12}C_{L4}~C_{M}U~U_{L12}~A_{F}~C_{M4}~U_{L}~U_{M}~A_{L}~A_{L}~G_{M}
P-U_{M}~U_{F}~G_{M}U_{L3}~C_{M}U_{F3}~C_{M4}U_{L3}~G_{M}G_{F}~U_{M3}C_{L4}~C_{M}U~U_{L3}~A_{F}~C_{M4}~U_{L3}~U_{M}~A_{L}~A_{L}~G_{M}
P-U_{M}~U_{F}~G_{M}U_{L18}~C_{M}U_{F18}~C_{M4}U_{L18}~G_{M}G_{F}~U_{M18}C_{L4}~C_{M}U~U_{L18}~A_{F}~C_{M4}~U_{L18}~U_{M}~A_{L}~A_{L}~G_{M}
P-U_{M}~U_{F}~G_{M}U_{L12}~C_{M4}U_{F}~C_{M4}U_{L12}~G_{M}G_{F}~U_{M12}C_{L4}~C_{M4}U~U_{L12}~A_{F}~C_{M4}~U_{L}~U_{M}~A_{L}~A_{L}~G_{M}
P-U_{M}~U_{F}~G_{M}U_{L3}~C_{M4}U_{F}~C_{M4}U_{L3}~G_{M}G_{F}~U_{M3}C_{L4}~C_{M4}U~U_{L3}~A_{F}~C_{M4}~U_{L}~U_{M}~A_{L}~A_{L}~G_{M}
P-U_{M}~U_{F}~G_{M}U_{L18}~C_{M4}U_{F}~C_{M4}U_{L18}~G_{M}G_{F}~U_{M18}C_{L4}~C_{M4}U~U_{L18}~A_{F}~C_{M4}~U_{L}~U_{M}~A_{L}~A_{L}~G_{M}
P-U_{M}~U_{F}~G_{M}U_{L12}~C_{M4}U_{F12}~C_{M4}U_{L12}~G_{M}G_{F}~U_{M12}C_{L4}~C_{M4}U~U_{L12}~A_{F}~C_{M4}~U_{L}~U_{M}~A_{L}~A_{L}~G_{M}
P-U_{M}~U_{F}~G_{M}U_{L3}~C_{M4}U_{F3}~C_{M4}U_{L3}~G_{M}G_{F}~U_{M3}C_{L4}~C_{M4}U~U_{L3}~A_{F}~C_{M4}~U_{L}~U_{M}~A_{L}~A_{L}~G_{M}
P-U_{M}~U_{F}~G_{M}U_{L18}~C_{M4}U_{F18}~C_{M4}U_{L18}~G_{M}G_{F}~U_{M18}C_{L4}~C_{M4}U~U_{L18}~A_{F}~C_{M4}~U_{L}~U_{M}~A_{L}~A_{L}~G_{M}
P-U_{M}~U_{F}~G_{M}U_{L12}~C_{M4}U_{F12}~C_{M4}U_{L12}~G_{M}G_{F}~U_{M12}C_{L4}~C_{M4}U~U_{L12}~A_{F}~C_{M4}~U_{L12}~U_{M}~A_{L}~A_{L}~G_{M}
P-U_{M}~U_{F}~G_{M}U_{L3}~C_{M4}U_{F3}~C_{M4}U_{L3}~G_{M}G_{F}~U_{M3}C_{L4}~C_{M4}U~U_{L3}~A_{F}~C_{M4}~U_{L3}~U_{M}~A_{L}~A_{L}~G_{M}
P-U_{M}~U_{F}~G_{M}U_{L18}~C_{M4}U_{F18}~C_{M4}U_{L18}~G_{M}G_{F}~U_{M18}C_{L4}~C_{M4}U~U_{L18}~A_{F}~C_{M4}~U_{L18}~U_{M}~A_{L}~A_{L}~G_{M}
P-U_{M}~U_{F}~G_{M}U_{L}~C_{M4}U_{F}~C_{M4}A_{L}~G_{M}G_{F}~U_{M}C_{L4}~C_{M4}U~U_{L}~A_{F}~C_{M4}~U_{L}~U_{M}~A_{L}~A_{L}~G_{M}
P-U_{M}~U_{F}~G_{M}U_{L}~C_{M4}U_{F}~C_{M4}U_{L}~A_{M}G_{F}~U_{M}C_{L4}~C_{M4}U~U_{L}~A_{F}~C_{M4}~U_{L}~U_{M}~A_{L}~A_{L}~G_{M}
P-U_{M}~U_{F}~G_{M}U_{F}~C_{L}U_{F}~C_{L}U_{F}~G_{M}G_{F}~U_{M}C_{F}~C_{L}U~U_{L}~A_{F}~C_{M}~U_{L}~U_{M}~A_{L}~A_{L}~G_{M}
P-U_{M}~U_{F}~G_{M}U_{F12}~C_{L}U_{F}~C_{L4}U_{F12}~G_{M}G_{F}~U_{M}C_{F}~C_{L4}U~U_{L12}~A_{F}~C_{M}~U_{L}~U_{M}~A_{L}~A_{L}~G_{M}
P-U_{M}~U_{F}~G_{M}U_{F3}~C_{L}U_{F}~C_{L4}U_{F3}~G_{M}G_{F}~U_{M}C_{F}~C_{L4}U~U_{L3}~A_{F}~C_{M}~U_{L}~U_{M}~A_{L}~A_{L}~G_{M}
P-U_{M}~U_{F}~G_{M}U_{F18}~C_{L}U_{F}~C_{L4}U_{F18}~G_{M}G_{F}~U_{M}C_{F}~C_{L4}U~U_{L18}~A_{F}~C_{M}~U_{L}~U_{M}~A_{L}~A_{L}~G_{M}
P-U_{M}~U_{F}~G_{M}U_{F12}~C_{L4}U_{F}~C_{L}U_{F12}~G_{M}G_{F}~U_{M}C_{F4}~C_{L}U~U_{L12}~A_{F}~C_{M4}~U_{L}~U_{M}~A_{L}~A_{L}~G_{M}
P-U_{M}~U_{F}~G_{M}U_{F3}~C_{L4}U_{F}~C_{L}U_{F3}~G_{M}G_{F}~U_{M}C_{F4}~C_{L}U~U_{L3}~A_{F}~C_{M4}~U_{L}~U_{M}~A_{L}~A_{L}~G_{M}
P-U_{M}~U_{F}~G_{M}U_{F18}~C_{L4}U_{F}~C_{L}U_{F18}~G_{M}G_{F}~U_{M}C_{F4}~C_{L}U~U_{L18}~A_{F}~C_{M4}~U_{L}~U_{M}~A_{L}~A_{L}~G_{M}
P-U_{M}~U_{F}~G_{M}U_{F12}~C_{L4}U_{F}~C_{L4}U_{F12}~G_{M}G_{F}~U_{M}C_{F4}~C_{L4}U~U_{L12}~A_{F}~C_{M4}~U_{L}~U_{M}~A_{L}~A_{L}~G_{M}
P-U_{M}~U_{F}~G_{M}U_{F3}~C_{L4}U_{F}~C_{L4}U_{F3}~G_{M}G_{F}~U_{M}C_{F4}~C_{L4}U~U_{L3}~A_{F}~C_{M4}~U_{L}~U_{M}~A_{L}~A_{L}~G_{M}
P-U_{M}~U_{F}~G_{M}U_{F18}~C_{L4}U_{F}~C_{L4}U_{F18}~G_{M}G_{F}~U_{M}C_{F4}~C_{L4}U~U_{L18}~A_{F}~C_{M4}~U_{L}~U_{M}~A_{L}~A_{L}~G_{M}
P-U_{M}~U_{F}~G_{M}U_{F12}~C_{L}U_{F}~C_{L}U_{F12}~G_{M}G_{F}~U_{M12}C_{F}~C_{L}U~U_{L12}~A_{F}~C_{M}~U_{L}~U_{M}~A_{L}~A_{L}~G_{M}
P-U_{M}~U_{F}~G_{M}U_{F3}~C_{L}U_{F}~C_{L}U_{F3}~G_{M}G_{F}~U_{M3}C_{F}~C_{L}U~U_{L3}~A_{F}~C_{M}~U_{L}~U_{M}~A_{L}~A_{L}~G_{M}
P-U_{M}~U_{F}~G_{M}U_{F18}~C_{L}U_{F}~C_{L}U_{F18}~G_{M}G_{F}~U_{M18}C_{F}~C_{L}U~U_{L18}~A_{F}~C_{M}~U_{L}~U_{M}~A_{L}~A_{L}~G_{M}
P-U_{M}~U_{F}~G_{M}U_{F12}~C_{L}U_{F12}~C_{L}U_{F12}~G_{M}G_{F}~U_{M12}C_{F}~C_{L}U~U_{L12}~A_{F}~C_{M}~U_{L}~U_{M}~A_{L}~A_{L}~G_{M}
P-U_{M}~U_{F}~G_{M}U_{F3}~C_{L}U_{F3}~C_{L}U_{F3}~G_{M}G_{F}~U_{M3}C_{F}~C_{L}U~U_{L3}~A_{F}~C_{M}~U_{L}~U_{M}~A_{L}~A_{L}~G_{M}
P-U_{M}~U_{F}~G_{M}U_{F18}~C_{L}U_{F18}~C_{L}U_{F18}~G_{M}G_{F}~U_{M18}C_{F}~C_{L}U~U_{L18}~A_{F}~C_{M}~U_{L}~U_{M}~A_{L}~A_{L}~G_{M}
P-U_{M}~U_{F}~G_{M}U_{F12}~C_{L}U_{F12}~C_{L}U_{F12}~G_{M}G_{F}~U_{M12}C_{F}~C_{L}U~U_{L12}~A_{F}~C_{M}~U_{L12}~U_{M}~A_{L}~A_{L}~G_{M}
P-U_{M}~U_{F}~G_{M}U_{F3}~C_{L}U_{F3}~C_{L}U_{F3}~G_{M}G_{F}~U_{M3}C_{F}~C_{L}U~U_{L3}~A_{F}~C_{M}~U_{L3}~U_{M}~A_{L}~A_{L}~G_{M}
P-U_{M}~U_{F}~G_{M}U_{F18}~C_{L}U_{F18}~C_{L}U_{F18}~G_{M}G_{F}~U_{M18}C_{F}~C_{L}U~U_{L18}~A_{F}~C_{M}~U_{L18}~U_{M}~A_{L}~A_{L}~G_{M}
P-U_{M}~U_{F}~G_{M}U_{F12}~C_{L}U_{F}~C_{L4}U_{F12}~G_{M}G_{F}~U_{M12}C_{F4}~C_{L}U~U_{L12}~A_{F}~C_{M4}~U_{L}~U_{M}~A_{L}~A_{L}~G_{M}
P-U_{M}~U_{F}~G_{M}U_{F3}~C_{L}U_{F}~C_{L4}U_{F3}~G_{M}G_{F}~U_{M3}C_{F4}~C_{L}U~U_{L3}~A_{F}~C_{M4}~U_{L}~U_{M}~A_{L}~A_{L}~G_{M}
P-U_{M}~U_{F}~G_{M}U_{F18}~C_{L}U_{F}~C_{L4}U_{F18}~G_{M}G_{F}~U_{M18}C_{F4}~C_{L}U~U_{L18}~A_{F}~C_{M4}~U_{L}~U_{M}~A_{L}~A_{L}~G_{M}
P-U_{M}~U_{F}~G_{M}U_{F12}~C_{L}U_{F12}~C_{L4}U_{F12}~G_{M}G_{F}~U_{M12}C_{F4}~C_{L}U~U_{L12}~A_{F}~C_{M4}~U_{L}~U_{M}~A_{L}~A_{L}~G_{M}
P-U_{M}~U_{F}~G_{M}U_{F3}~C_{L}U_{F3}~C_{L4}U_{F3}~G_{M}G_{F}~U_{M3}C_{F4}~C_{L}U~U_{L3}~A_{F}~C_{M4}~U_{L}~U_{M}~A_{L}~A_{L}~G_{M}
P-U_{M}~U_{F}~G_{M}U_{F18}~C_{L}U_{F18}~C_{L4}U_{F18}~G_{M}G_{F}~U_{M18}C_{F4}~C_{L}U~U_{L18}~A_{F}~C_{M4}~U_{L}~U_{M}~A_{L}~A_{L}~G_{M}
P-U_{M}~U_{F}~G_{M}U_{F12}~C_{L}U_{F12}~C_{L4}U_{F12}~G_{M}G_{F}~U_{M12}C_{F4}~C_{F}U~U_{F12}~A_{F}~C_{M4}~U_{F12}~U_{M}~A_{L}~A_{L}~G_{M}
P-U_{M}~U_{F}~G_{M}U_{F3}~C_{L}U_{F3}~C_{L4}U_{F3}~G_{M}G_{F}~U_{M3}C_{F4}~C_{L}U~U_{L3}~A_{F}~C_{M4}~U_{L3}~U_{M}~A_{L}~A_{L}~G_{M}
P-U_{M}~U_{F}~G_{M}U_{F18}~C_{L}U_{F18}~C_{L4}U_{F18}~G_{M}G_{F}~U_{M18}C_{F4}~C_{L}U~U_{L18}~A_{F}~C_{M4}~U_{L18}~U_{M}~A_{L}~A_{L}~G_{M}
P-U_{M}~U_{F}~G_{M}U_{F12}~C_{L4}U_{F}~C_{L4}U_{F12}~G_{M}G_{F}~U_{M12}C_{F4}~C_{L4}U~U_{L12}~A_{F}~C_{M4}~U_{L}~U_{M}~A_{L}~A_{L}~G_{M}
P-U_{M}~U_{F}~G_{M}U_{F3}~C_{L4}U_{F}~C_{L4}U_{F3}~G_{M}G_{F}~U_{M3}C_{F4}~C_{L4}U~U_{L3}~A_{F}~C_{M4}~U_{L}~U_{M}~A_{L}~A_{L}~G_{M}
P-U_{M}~U_{F}~G_{M}U_{F18}~C_{L4}U_{F}~C_{L4}U_{F18}~G_{M}G_{F}~U_{M18}C_{F4}~C_{L4}U~U_{L18}~A_{F}~C_{M4}~U_{L}~U_{M}~A_{L}~A_{L}~G_{M}
P-U_{M}~U_{F}~G_{M}U_{F12}~C_{L4}U_{F12}~C_{L4}U_{F12}~G_{M}G_{F}~U_{M12}C_{F4}~C_{L4}U~U_{L12}~A_{F}~C_{M4}~U_{L}~U_{M}~A_{L}~A_{L}~G_{M}
P-U_{M}~U_{F}~G_{M}U_{F3}~C_{L4}U_{F3}~C_{L4}U_{F3}~G_{M}G_{F}~U_{M3}C_{F4}~C_{L4}U~U_{L3}~A_{F}~C_{M4}~U_{L}~U_{M}~A_{L}~A_{L}~G_{M}
P-U_{M}~U_{F}~G_{M}U_{F18}~C_{L4}U_{F18}~C_{L4}U_{F18}~G_{M}G_{F}~U_{M18}C_{F4}~C_{L4}U~U_{L18}~A_{F}~C_{M4}~U_{L}~U_{M}~A_{L}~A_{L}~G_{M}
P-U_{M}~U_{F}~G_{M}U_{F12}~C_{L4}U_{F12}~C_{L4}U_{F12}~G_{M}G_{F}~U_{M12}C_{F4}~C_{L4}U~U_{L12}~A_{F}~C_{M4}~U_{L12}~U_{M}~A_{L}~A_{L}~G_{M}
P-U_{M}~U_{F}~G_{M}U_{F3}~C_{L4}U_{F3}~C_{L4}U_{F3}~G_{M}G_{F}~U_{M3}C_{F4}~C_{L4}U~U_{L3}~A_{F}~C_{M4}~U_{L3}~U_{M}~A_{L}~A_{L}~G_{M}
P-U_{M}~U_{F}~G_{M}U_{F18}~C_{L4}U_{F18}~C_{L4}U_{F18}~G_{M}G_{F}~U_{M18}C_{F4}~C_{L4}U~U_{L18}~A_{F}~C_{M4}~U_{L18}~U_{M}~A_{L}~A_{L}~G_{M}
P-U_{M}~U_{F}~G_{M}U_{F}~C_{L4}U_{F}~C_{L4}A_{F}~G_{M}G_{F}~U_{M}C_{F4}~C_{L4}U~U_{L}~A_{F}~C_{M4}~U_{L}~U_{M}~A_{L}~A_{L}~G_{M}
P-U_{M}~U_{F}~G_{M}U_{F}~C_{L4}U_{F}~C_{L4}U_{F}~A_{M}G_{F}~U_{M}C_{F4}~C_{L4}U~U_{L}~A_{F}~C_{M4}~U_{L}~U_{M}~A_{L}~A_{L}~G_{M}

### Table 9

### Examples of Murine/Human PTEN ags-siRNA Compositions

### A. Murine/Human PTEN Antisense Sequence:

UGAACAUUGGAAUAGUUUC

Lima et al. (Cell 150: 883-94, 2012) used this sequence to generate ssRNAi compounds against human/murine PTEN including the following:

5′-VP-Tₘₒₑ~G_{F}~A_{M}A_{F}~C_{M}A_{F}~U_{M}U_{F}~G_{M}G_{F}~
A_{M}A_{F}~U_{M}A_{F}~G_{M}~U_{F}~U_{M}~U_{F}~C_{M}~Aₘₒₑ~Aₘₒₑ

Any of the 5′-end nucleosides (P-U_{M}) shown in the examples of agsRNAi compounds in Table 9 can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-A_{F}, P-A_{M}, P-A_{N}, P-A_{L}, P-A_{Z}, P-A_{H}, P-A_{B}, P-A_{K}, P-A_{Q}, P-A_{S}, P-A_{T}, P-A_{W}, P-A_{V}, P-U_{F}, P-U_{M}, P-U_{L}, P-U_{Z}, P-U_{H}, P-U_{H}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 9 with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the compounds in Table 9 can any of those units and linkages provided for herein for overhang precursors.

B. Examples of agsRNAi Compounds:

Compositions of Matter Suitable for Administration to a Subject
with or without a Protective Carrier and for Any Other use as
Provided for Herein (5′-3′)

P-U_{M}~G_{F}~A_{M}A_{F}~C_{M4}A_{F}~U_{M3}U_{F3}~G_{M}G_{F}~A_{M}A_{F}~U_{M}A_{F}~G_{M}~U_{F3}~U_{M3}~U_{F}~C_{M4}~A_{F}~G_{M}
P-U_{M}~G_{F}~A_{M}A_{F}~C_{M4}A_{F}~U_{M3}U_{F3}~G_{M}G_{F}~A_{M}A_{F}~U_{M3}A_{F}~G_{M}~U_{F3}~U_{M3}~U_{F3}~C_{M4}~A_{F}~G_{M}
P-U_{M}~G_{F}~A_{M}A_{F}~C_{M4}A_{F}~U_{M12}U_{F12}~G_{M}G_{F}~A_{M}A_{F}~U_{M}A_{F}~G_{M}~U_{F12}~U_{M12}~U_{F}~C_{M4}~A_{F}~G_{M}
P-U_{M}~G_{F}~A_{M}A_{F}~C_{M4}A_{F}~U_{M12}U_{F12}~G_{M}G_{F}~A_{M}A_{F}~U_{M12}A_{F}~G_{M}~U_{F12}~U_{M12}~U_{F12}~C_{M4}~A_{F}~G_{M}
P-U_{M}~G_{F}~A_{M}A_{F}~C_{M4}A_{F}~U_{M18}U_{F18}~G_{M}G_{F}~A_{M}A_{F}~U_{M}A_{F}~G_{M}~U_{F18}~U_{M18}~U_{F}~C_{M4}~A_{F}~G_{M}
P-U_{M}~G_{F}~A_{M}A_{F}~C_{M4}A_{F}~U_{M18}U_{F18}~G_{M}G_{F}~A_{M}A_{F}~U_{M18}A_{F}~G_{M}~U_{F18}~U_{M18}~U_{F18}~C_{M4}~A_{F}~G_{M}
P-U_{M}~G_{F}~A_{M}A_{F2}~C_{M4}A_{F}~U_{M3}U_{F3}~G_{M}G_{F}~A_{M}A_{F}~U_{M}A_{F}~G_{M}~U_{F3}~U_{M3}~U_{F}~C_{M4}~A_{F}~G_{M}
P-U_{M}~G_{F}~A_{M}A_{F2}~C_{M4}A_{F}~U_{M3}U_{F3}~G_{M}G_{F}~A_{M}A_{F}~U_{M3}A_{F}~G_{M}~U_{F3}~U_{M3}~U_{F3}~C_{M4}~A_{F}~G_{M}
P-U_{M}~G_{F}~A_{M}A_{F2}~C_{M}A_{F}~U_{M3}U_{F3}~G_{M}G_{F}~A_{M}A_{F}~U_{M}A_{F}~G_{M}~U_{F3}~U_{M3}~U_{F3}~C_{M}~A_{F}~G_{M}
P-U_{M}~G_{F}~A_{M}A_{F2}~C_{M}A_{F}~U_{M3}U_{F3}~G_{M}G_{F}~A_{M}A_{F}~U_{M3}A_{F}~G_{M}~U_{F3}~U_{M3}~U_{F3}~C_{M}~A_{F}~G_{M}
P-U_{M}~G_{F}~A_{M}A_{F2}~C_{M4}A_{F}~U_{M12}U_{F12}~G_{M}G_{F}~A_{M}A_{F}~U_{M}A_{F}~G_{M}~U_{F12}~U_{M12}~U_{F3}~C_{M4}~A_{F}~G_{M}
P-U_{M}~G_{F}~A_{M}A_{F2}~C_{M4}A_{F}~U_{M12}U_{F12}~G_{M}G_{F}~A_{M}A_{F}~U_{M12}A_{F}~G_{M}~U_{F12}~U_{M12}~U_{F12}~C_{M4}~A_{F}~G_{M}
P-U_{M}~G_{F}~A_{M}A_{F2}~C_{M}A_{F}~U_{M12}U_{F12}~G_{M}G_{F}~A_{M}A_{F}~U_{M}A_{F}~G_{M}~U_{F12}~U_{M12}~U_{F12}~C_{M}~A_{F}~G_{M}
P-U_{M}~G_{F}~A_{M}A_{F2}~C_{M}A_{F}~U_{M12}U_{F12}~G_{M}G_{F}~A_{M}A_{F}~U_{M12}A_{F}~G_{M}~U_{F12}~U_{M12}~U_{F12}~C_{M}~A_{F}~G_{M}
P-U_{M}~G_{F}~A_{M}A_{F2}~C_{M4}A_{F}~U_{M18}U_{F18}~G_{M}G_{F}~A_{M}A_{F}~U_{M}A_{F}~G_{M}~U_{F18}~U_{M18}~U_{F18}~C_{M4}~A_{F}~G_{M}
P-U_{M}~G_{F}~A_{M}A_{F2}~C_{M4}A_{F}~U_{M18}U_{F18}~G_{M}G_{F}~A_{M}A_{F}~U_{M18}A_{F}~G_{M}~U_{F18}~U_{M18}~U_{F18}~C_{M4}~A_{F}~G_{M}
P-U_{M}~G_{F}~A_{M}A_{F2}~C_{M}A_{F}~U_{M18}U_{F18}~G_{M}G_{F}~A_{M}A_{F}~U_{M}A_{F}~G_{M}~U_{F18}~U_{M18}~U_{F18}~C_{M}~A_{F}~G_{M}
P-U_{M}~G_{F}~A_{M}A_{F2}~C_{M}A_{F}~U_{M18}U_{F18}~G_{M}G_{F}~A_{M}A_{F}~U_{M18}A_{F}~G_{M}~U_{F18}~U_{M18}~U_{F18}~C_{M}~A_{F}~G_{M}
P-U_{M}~G_{F}~A_{M}A_{F}~C_{M4}A_{F}~U_{M3}U_{F3}~G_{M}G_{F}~A_{M}A_{F}~U_{M}A~G_{M}~U_{F3}~U_{M3}~U_{F}~C_{M4}~A_{F}~G_{M}
P-U_{M}~G_{F}~A_{M}A_{F}~C_{M4}A_{F}~U_{M3}U_{F3}~G_{M}G_{F}~A_{M}A_{F}~U_{M3}A~G_{M}~U_{F3}~U_{M3}~U_{F3}~C_{M4}~A_{F}~G_{M}
P-U_{M}~G_{F}~A_{M}A_{F}~C_{M4}A_{F}~U_{M12}U_{F12}~G_{M}G_{F}~A_{M}A_{F}~U_{M}A~G_{M}~U_{F12}~U_{M12}~U_{F}~C_{M4}~A_{F}~G_{M}
P-U_{M}~G_{F}~A_{M}A_{F}~C_{M4}A_{F}~U_{M12}U_{F12}~G_{M}G_{F}~A_{M}A_{F}~U_{M12}A~G_{M}~U_{F12}~U_{M12}~U_{F12}~C_{M4}~A_{F}~G_{M}
P-U_{M}~G_{F}~A_{M}A_{F}~C_{M4}A_{F}~U_{M18}U_{F18}~G_{M}G_{F}~A_{M}A_{F}~U_{M}A~G_{M}~U_{F18}~U_{M18}~U_{F}~C_{M4}~A_{F}~G_{M}
P-U_{M}~G_{F}~A_{M}A_{F}~C_{M4}A_{F}~U_{M18}U_{F18}~G_{M}G_{F}~A_{M}A_{F}~U_{M18}A~G_{M}~U_{F18}~U_{M18}~U_{F18}~C_{M4}~A_{F}~G_{M}
P-U_{M}~G_{F}~A_{M}A_{F2}~C_{M4}A_{F}~U_{M3}U_{F3}~G_{M}G_{F}~A_{M}A_{F}~U_{M}A~G_{M}~U_{F3}~U_{M3}~U_{F}~C_{M4}~A_{F}~G_{M}
P-U_{M}~G_{F}~A_{M}A_{F2}~C_{M4}A_{F}~U_{M3}U_{F3}~G_{M}G_{F}~A_{M}A_{F}~U_{M3}A~G_{M}~U_{F3}~U_{M3}~U_{F3}~C_{M4}~A_{F}~G_{M}
P-U_{M}~G_{F}~A_{M}A_{F2}~C_{M}A_{F}~U_{M3}U_{F3}~G_{M}G_{F}~A_{M}A_{F}~U_{M}A~G_{M}~U_{F3}~U_{M3}~U_{F3}~C_{M}~A_{F}~G_{M}
P-U_{M}~G_{F}~A_{M}A_{F2}~C_{M}A_{F}~U_{M3}U_{F3}~G_{M}G_{F}~A_{M}A_{F}~U_{M3}A~G_{M}~U_{F3}~U_{M3}~U_{F3}~C_{M}~A_{F}~G_{M}
P-U_{M}~G_{F}~A_{M}A_{F2}~C_{M4}A_{F}~U_{M12}U_{F12}~G_{M}G_{F}~A_{M}A_{F}~U_{M}A~G_{M}~U_{F12}~U_{M12}~U_{F3}~C_{M4}~A_{F}~G_{M}
P-U_{M}~G_{F}~A_{M}A_{F2}~C_{M4}A_{F}~U_{M12}U_{F12}~G_{M}G_{F}~A_{M}A_{F}~U_{M12}A~G_{M}~U_{F12}~U_{M12}~U_{F12}~C_{M4}~A_{F}~G_{M}
P-U_{M}~G_{F}~A_{M}A_{F2}~C_{M}A_{F}~U_{M12}U_{F12}~G_{M}G_{F}~A_{M}A_{F}~U_{M}A~G_{M}~U_{F12}~U_{M12}~U_{F12}~C_{M}~A_{F}~G_{M}
P-U_{M}~G_{F}~A_{M}A_{F2}~C_{M}A_{F}~U_{M12}U_{F12}~G_{M}G_{F}~A_{M}A_{F}~U_{M12}A~G_{M}~U_{F12}~U_{M12}~U_{F12}~C_{M}~A_{F}~G_{M}
P-U_{M}~G_{F}~A_{M}A_{F2}~C_{M4}A_{F}~U_{M18}U_{F18}~G_{M}G_{F}~A_{M}A_{F}~U_{M}A~G_{M}~U_{F18}~U_{M18}~U_{F18}~C_{M4}~A_{F}~G_{M}
P-U_{M}~G_{F}~A_{M}A_{F2}~C_{M4}A_{F}~U_{M18}U_{F18}~G_{M}G_{F}~A_{M}A_{F}~U_{M18}A~G_{M}~U_{F18}~U_{M18}~U_{F18}~C_{M4}~A_{F}~G_{M}
P-U_{M}~G_{F}~A_{M}A_{F2}~C_{M}A_{F}~U_{M18}U_{F18}~G_{M}G_{F}~A_{M}A_{F}~U_{M18}~G_{M}~U_{F18}~U_{M18}~U_{F18}~C_{M}~A_{F}~G_{M}
P-U_{M}~G_{F}~A_{M}A_{F2}~C_{M}A_{F}~U_{M18}U_{F18}~G_{M}G_{F}~A_{M}A_{F}~U_{M18}A~G_{M}~U_{F18}~U_{M18}~U_{F18}~C_{M}~A_{F}~G_{M}
P-U_{M}~G_{F}~A_{L}A_{F}~C_{M}A_{L}~U_{M}U_{F}~G_{L}G_{F}~A_{M}A_{L}~U_{M}A_{F}~G_{L}~U_{F}~U_{M}~U_{L}~C_{M}~A_{F}~G_{M}
P-U_{M}~G_{F}~A_{L}A_{F}~C_{M}A_{L}~U_{M}U_{F}~G_{L}G_{F}~A_{M}A_{L}~U_{M}A~G_{L}~U_{F}~U_{M}~U_{L}~C_{M}~A_{F}~G_{M}
P-U_{M}~G_{F}~A_{L}A_{F}~C_{M4}A_{L}~U_{M3}U_{F3}~G_{L}G_{F}~A_{M}A_{L}~U_{M}A_{F}~G_{L}~U_{F3}~U_{M3}~U_{L}~C_{M4}~A_{F}~G_{M}
P-U_{M}~G_{F}~A_{L}A_{F}~C_{M4}A_{L}~U_{M3}U_{F3}~G_{L}G_{F}~A_{M}A_{L}~U_{M3}A_{F}~G_{L}~U_{F3}~U_{M3}~U_{L3}~C_{M4}~A_{F}~G_{M}
P-U_{M}~G_{F}~A_{L}A_{F}~C_{M4}A_{L}~U_{M12}U_{F12}~G_{L}G_{F}~A_{M}A_{L}~U_{M}A_{F}~G_{L}~U_{F12}~U_{M12}~U_{L}~C_{M4}~A_{F}~G_{M}
P-U_{M}~G_{F}~A_{L}A_{F}~C_{M4}A_{L}~U_{M12}U_{F12}~G_{L}G_{F}~A_{M}A_{L}~U_{M12}A_{F}~G_{L}~U_{F12}~U_{M12}~U_{L12}~C_{M4}~A_{F}~G_{M}
P-U_{M}~G_{F}~A_{L}A_{F}~C_{M4}A_{L}~U_{M18}U_{F18}~G_{L}G_{F}~A_{M}A_{L}~U_{M}A_{F}~G_{L}~U_{F18}~U_{M18}~U_{L}~C_{M4}~A_{F}~G_{M}
P-U_{M}~G_{F}~A_{L}A_{F}~C_{M4}A_{L}~U_{M18}U_{F18}~G_{L}G_{F}~A_{M}A_{L}~U_{M18}A_{F}~G_{L}~U_{F18}~U_{M18}~U_{L18}~C_{M4}~A_{F}~G_{M}
P-U_{M}~G_{F}~A_{L}A_{F2}~C_{M4}A_{L}~U_{M3}U_{F3}~G_{L}G_{F}~A_{M}A_{L}~U_{M}A_{F}~G_{L}~U_{F3}~U_{M3}~U_{L}~C_{M4}~A_{F}~G_{M}
P-U_{M}~G_{F}~A_{L}A_{F2}~C_{M4}A_{L}~U_{M3}U_{F3}~G_{L}G_{F}~A_{M}A_{L}~U_{M3}A_{F}~G_{L}~U_{F3}~U_{M3}~U_{L3}~C_{M4}~A_{F}~G_{M}
P-U_{M}~G_{F}~A_{L}A_{F2}~C_{M}A_{L}~U_{M3}U_{F3}~G_{L}G_{F}~A_{M}A_{L}~U_{M}A_{F}~G_{L}~U_{F3}~U_{M3}~U_{L3}~C_{M}~A_{F}~G_{M}
P-U_{M}~G_{F}~A_{L}A_{F2}~C_{M}A_{L}~U_{M3}U_{F3}~G_{L}G_{F}~A_{M}A_{L}~U_{M3}A_{F}~G_{U}~U_{F3}~U_{M3}~U_{L3}~C_{M}~A_{F}~G_{M}
P-U_{M}~G_{F}~A_{L}A_{F2}~C_{M4}A_{L}~U_{M12}U_{F12}~G_{L}G_{F}~A_{M}A_{L}~U_{M}A_{F}~G_{L}~U_{F12}~U_{M12}~U_{L3}~C_{M4}~A_{F}~G_{M}
P-U_{M}~G_{F}~A_{L}A_{F2}~C_{M4}A_{L}~U_{M12}U_{F12}~G_{L}G_{F}~A_{M}A_{L}~U_{M12}A_{F}~G_{L}~U_{F12}~U_{M12}~U_{L12}~C_{M4}~A_{F}~G_{M}
P-U_{M}~G_{F}~A_{L}A_{F2}~C_{M}A_{L}~U_{M12}U_{F12}~G_{L}G_{F}~A_{M}A_{L}~U_{M}A_{F}~G_{L}~U_{F12}~U_{M12}~U_{L12}~C_{M}~A_{F}~G_{M}
P-U_{M}~G_{F}~A_{L}A_{F2}~C_{M}A_{L}~U_{M12}U_{F12}~G_{L}G_{F}~A_{M}A_{L}~U_{M12}A_{F}~G_{L}~U_{F12}~U_{M12}~U_{L12}~C_{M}~A_{F}~G_{M}
P-U_{M}~G_{F}~A_{L}A_{F2}~C_{M4}A_{L}~U_{M18}U_{F18}~G_{L}G_{F}~A_{M}A_{L}~U_{M}A_{F}~G_{L}~U_{F18}~U_{M18}~U_{L18}~C_{M4}~A_{F}~G_{M}
P-U_{M}~G_{F}~A_{L}A_{F2}~C_{M4}A_{L}~U_{M18}U_{F18}~G_{L}G_{F}~A_{M}A_{L}~U_{M18}A_{F}~G_{L}~U_{F18}~U_{M18}~U_{L18}~C_{M4}~A_{F}~G_{M}
P-U_{M}~G_{F}~A_{L}A_{F2}~C_{M}A_{L}~U_{M18}U_{F18}~G_{L}G_{F}~A_{M}A_{L}~U_{M}A_{F}~G_{L}~U_{F18}~U_{M18}~U_{L18}~C_{M}~A_{F}~G_{M}
P-U_{M}~G_{F}~A_{L}A_{F2}~C_{M}A_{L}~U_{M18}U_{F18}~G_{L}G_{F}~A_{M}A_{L}~U_{M18}A_{F}~G_{L}~U_{F18}~U_{M18}~U_{L18}~C_{M}~A_{F}~G_{M}
P-U_{M}~G_{F}~A_{L}A_{F}~C_{M4}A_{L}~U_{M3}U_{F3}~G_{L}G_{F}~A_{M}A_{L}~U_{M}A~G_{L}~U_{F3}~U_{M3}~U_{L}~C_{M4}~A_{F}~G_{M}
P-U_{M}~G_{F}~A_{L}A_{F}~C_{M4}A_{L}~U_{M3}U_{F3}~G_{L}G_{F}~A_{M}A_{L}~U_{M3}A~G_{L}~U_{F3}~U_{M3}~U_{L3}~C_{M4}~A_{F}~G_{M}
P-U_{M}~G_{F}~A_{L}A_{F}~C_{M4}A_{L}~U_{M12}U_{F12}~G_{L}G_{F}~A_{M}A_{L}~U_{M}A~G_{L}~U_{F12}~U_{M12}~U_{L}~C_{M4}~A_{F}~G_{M}
P-U_{M}~G_{F}~A_{L}A_{F}~C_{M4}A_{L}~U_{M12}U_{F12}~G_{L}G_{F}~A_{M}A_{L}~U_{M12}A~G_{L}~U_{F12}~U_{M12}~U_{L12}~C_{M4}~A_{F}~G_{M}
P-U_{M}~G_{F}~A_{L}A_{F}~C_{M4}A_{L}~U_{M18}U_{F18}~G_{L}G_{F}~A_{M}A_{L}~U_{M}A~G_{U}~U_{F18}~U_{M18}~U_{L}~C_{M4}~A_{F}~G_{M}
P-U_{M}~G_{F}~A_{L}A_{F}~C_{M4}A_{L}~U_{M18}U_{F18}~G_{L}G_{F}~A_{M}A_{L}~U_{M18}A~G_{L}~U_{F18}~U_{M18}~U_{L18}~C_{M4}~A_{F}~G_{M}
P-U_{M}~G_{F}~A_{L}A_{F2}~C_{M4}A_{L}~U_{M3}U_{F3}~G_{L}G_{F}~A_{M}A_{L}~U_{M}A~G_{L}~U_{F3}~U_{M3}~U_{L}~C_{M4}~A_{F}~G_{M}
P-U_{M}~G_{F}~A_{L}A_{F2}~C_{M4}A_{L}~U_{M3}U_{F3}~G_{L}G_{F}~A_{M}A_{L}~U_{M3}A~G_{L}~U_{F3}~U_{M3}~U_{L3}~C_{M4}~A_{F}~G_{M}
P-U_{M}~G_{F}~A_{L}A_{F2}~C_{M}A_{L}~U_{M3}U_{F3}~G_{L}G_{F}~A_{M}A_{L}~U_{M}A~G_{U}~U_{F3}~U_{M3}~U_{L3}~C_{M}~A_{F}~G_{M}
P-U_{M}~G_{F}~A_{L}A_{F2}~C_{M}A_{L}~U_{M3}U_{F3}~G_{L}G_{F}~A_{M}A_{L}~U_{M3}A~G_{L}~U_{F3}~U_{M3}~U_{L3}~C_{M}~A_{F}~G_{M}
P-U_{M}~G_{F}~A_{L}A_{F2}~C_{M4}A_{L}~U_{M12}U_{F12}~G_{L}G_{F}~A_{M}A_{L}~U_{M}A~G_{L}~U_{F12}~U_{M2}~U_{L3}~C_{M4}~A_{F}~G_{M}
P-U_{M}~G_{F}~A_{L}A_{F2}~C_{M4}A_{L}~U_{M12}U_{F12}~G_{L}G_{F}~A_{M}A_{L}~U_{M12}A~G_{L}~U_{F12}~U_{M12}~U_{L12}~C_{M4}~A_{F}~G_{M}
P-U_{M}~G_{F}~A_{L}A_{F2}~C_{M}A_{L}~U_{M12}U_{F12}~G_{L}G_{F}~A_{M}A_{L}~U_{M}A~G_{L}~U_{F12}~U_{M12}~U_{L12}~C_{M}~A_{F}~G_{M}
P-U_{M}~G_{F}~A_{L}A_{F2}~C_{M}A_{L}~U_{M12}U_{F12}~G_{L}G_{F}~A_{M}A_{L}~U_{M12}A~G_{L}~U_{F12}~U_{M12}~U_{L12}~C_{M}~A_{F}~G_{M}
P-U_{M}~G_{F}~A_{L}A_{F2}~C_{M4}A_{L}~U_{M18}U_{F18}~G_{L}G_{F}~A_{M}A_{L}~U_{M}A~G_{L}~U_{F18}~U_{M18}~U_{L18}~C_{M4}~A_{F}~G_{M}
P-U_{M}~G_{F}~A_{L}A_{F2}~C_{M4}A_{L}~U_{M18}U_{F18}~G_{L}G_{F}~A_{M}A_{L}~U_{M18}A~G_{U}~U_{F18}~U_{M18}~U_{L18}~C_{M4}~A_{F}~G_{M}
P-U_{M}~G_{F}~A_{L}A_{F2}~C_{M}A_{L}~U_{M18}U_{F18}~G_{L}G_{F}~A_{M}A_{L}~U_{M}A~G_{L}~U_{F18}~U_{M18}~U_{L18}~C_{M}~A_{F}~G_{M}
P-U_{M}~G_{F}~A_{L}A_{F2}~C_{M}A_{L}~U_{M18}U_{F18}~G_{L}G_{F}~A_{M}A_{L}~U_{M18}A~G_{L}~U_{F18}~U_{M18}~U_{L18}~C_{M}~A_{F}~G_{M}
P-U_{M}~G_{F}~A_{M}A_{L}~C_{M}A_{F}~U_{M}U_{F}~G_{L}G_{F}~A_{M}A_{F}~U_{L}A_{F}~G_{M}~U_{F}~U_{L}~U_{F}~C_{M}~A_{F}~G_{M}
P-U_{M}~G_{F}~A_{M}A_{L}~C_{M}A_{F}~U_{M}U_{F}~G_{L}G_{F}~A_{M}A_{F}~U_{L}A~G_{M}~U_{F}~U_{L}~U_{F}~C_{M}~A_{F}~G_{M}
P-U_{M}~G_{F}~A_{M}A_{L}~C_{M4}A_{F}~U_{M3}U_{F3}~G_{L}G_{F}~A_{M}A_{F}~U_{L}A_{F}~G_{M}~U_{F3}~U_{L3}~U_{F}~C_{M4}~A_{F}~G_{M}
P-U_{M}~G_{F}~A_{M}A_{L}~C_{M4}A_{F}~U_{M3}U_{F3}~G_{L}G_{F}~A_{M}A_{F}~U_{L3}A_{F}~G_{M}~U_{F3}~U_{L3}~U_{F3}~C_{M4}~A_{F}~G_{M}
P-U_{M}~G_{F}~A_{M}A_{L}~C_{M4}A_{F}~U_{M12}U_{F12}~G_{L}G_{F}~A_{M}A_{F}~U_{L}A_{F}~G_{M}~U_{F12}~U_{L12}~U_{F}~C_{M4}~A_{F}~G_{M}
P-U_{M}~G_{F}~A_{M}A_{L}~C_{M4}A_{F}~U_{M12}U_{F12}~G_{L}G_{F}~A_{M}A_{F}~U_{L12}A_{F}~G_{M}~U_{F12}~U_{L12}~U_{F12}~C_{M4}~A_{F}~G_{M}
P-U_{M}~G_{F}~A_{M}A_{L}~C_{M4}A_{F}~U_{M18}U_{F18}~G_{L}G_{F}~A_{M}A_{F}~U_{L}A_{F}~G_{M}~U_{F18}~U_{L18}~U_{F}~C_{M4}~A_{F}~G_{M}
P-U_{M}~G_{F}~A_{M}A_{L}~C_{M4}A_{F}~U_{M18}U_{F18}~G_{L}G_{F}~A_{M}A_{F}~U_{L18}A_{F}~G_{M}~U_{L18}~U_{M18}~U_{F18}~C_{M4}~A_{F}~G_{M}
P-U_{M}~G_{F}~A_{M}A_{L2}~C_{M4}A_{F}~U_{M3}U_{F3}~G_{L}G_{F}~A_{M}A_{F}~U_{L}A_{F}~G_{M}~U_{F3}~U_{L3}~U_{F}~C_{M4}~A_{F}~G_{M}
P-U_{M}~G_{F}~A_{M}A_{L2}~C_{M4}A_{F}~U_{M3}U_{F3}~G_{L}G_{F}~A_{M}A_{F}~U_{L3}A_{F}~G_{M}~U_{F3}~U_{L3}~U_{F3}~C_{M4}~A_{F}~G_{M}
P-U_{M}~G_{F}~A_{M}A_{L2}~C_{M}A_{F}~U_{M3}U_{F3}~G_{L}G_{F}~A_{M}A_{F}~U_{L}A_{F}~G_{M}~U_{F3}~U_{L3}~U_{F3}~C_{M}~A_{F}~G_{M}
P-U_{M}~G_{F}~A_{M}A_{L2}~C_{M}A_{F}~U_{M3}U_{F3}~G_{L}G_{F}~A_{M}A_{F}~U_{L3}A_{F}~G_{M}~U_{F3}~U_{L3}~U_{F3}~C_{M}~A_{F}~G_{M}
P-U_{M}~G_{F}~A_{M}A_{L2}~C_{M4}A_{F}~U_{M12}U_{F12}~G_{L}G_{F}~A_{M}A_{F}~U_{L}A_{F}~G_{M}~U_{F12}~U_{L12}~U_{F3}~C_{M4}~A_{F}~G_{M}
P-U_{M}~G_{F}~A_{M}A_{L2}~C_{M4}A_{F}~U_{M12}U_{F12}~G_{L}G_{F}~A_{M}A_{F}~U_{L12}A_{F}~G_{M}~U_{F12}~U_{L12}~U_{F12}~C_{M4}~A_{F}~G_{M}
P-U_{M}~G_{F}~A_{M}A_{L2}~C_{M}A_{F}~U_{M12}U_{F12}~G_{L}G_{F}~A_{M}A_{F}~U_{L}A_{F}~G_{M}~U_{F12}~U_{L12}~U_{F12}~C_{M}~A_{F}~G_{M}
P-U_{M}~G_{F}~A_{M}A_{L2}~C_{M}A_{F}~U_{M12}U_{F12}~G_{L}G_{F}~A_{M}A_{F}~U_{L12}A_{F}~G_{M}~U_{F12}~U_{L12}~U_{F12}~C_{M}~A_{F}~G_{M}
P-U_{M}~G_{F}~A_{M}A_{L2}~C_{M4}A_{F}~U_{M18}U_{F18}~G_{L}G_{F}~A_{M}A_{F}~U_{L}A_{F}~G_{M}~U_{F18}~U_{L18}~U_{F18}~C_{M4}~A_{F}~G_{M}
P-U_{M}~G_{F}~A_{M}A_{L2}~C_{M4}A_{F}~U_{M18}U_{F18}~G_{L}G_{F}~A_{M}A_{F}~U_{L18}A_{F}~G_{M}~U_{F18}~U_{L18}~U_{F18}~C_{M4}~A_{F}~G_{M}
P-U_{M}~G_{F}~A_{M}A_{L2}~C_{M}A_{F}~U_{M18}U_{F18}~G_{L}G_{F}~A_{M}A_{F}~U_{L}A_{F}~G_{M}~U_{F18}~U_{L18}~U_{F18}~C_{M}~A_{F}~G_{M}
P-U_{M}~G_{F}~A_{M}A_{L2}~C_{M}A_{F}~U_{M18}U_{F18}~G_{L}G_{F}~A_{M}A_{F}~U_{L18}A_{F}~G_{M}~U_{F18}~U_{L18}~U_{F18}~C_{M}~A_{F}~G_{M}
P-U_{M}~G_{F}~A_{M}A_{L}~C_{M4}A_{F}~U_{M3}U_{F3}~G_{L}G_{F}~A_{M}A_{F}~U_{L}A~G_{M}~U_{F3}~U_{L3}~U_{F}~C_{M4}~A_{F}~G_{M}
P-U_{M}~G_{F}~A_{M}A_{L}~C_{M4}A_{F}~U_{M3}U_{F3}~G_{L}G_{F}~A_{M}A_{F}~U_{L3}A~G_{M}~U_{F3}~U_{L3}~U_{F3}~C_{M4}~A_{F}~G_{M}
P-U_{M}~G_{F}~A_{M}A_{L}~C_{M4}A_{F}~U_{M12}U_{F12}~G_{L}G_{F}~A_{M}A_{F}~U_{L}A~G_{M}~U_{F12}~U_{L12}~U_{F}~C_{M4}~A_{F}~G_{M}
P-U_{M}~G_{F}~A_{M}A_{L}~C_{M4}A_{F}~U_{M12}U_{F12}~G_{L}G_{F}~A_{M}A_{F}~U_{L12}A~G_{M}~U_{F12}~U_{L12}~U_{F12}~C_{M4}~A_{F}~G_{M}
P-U_{M}~G_{F}~A_{M}A_{L}~C_{M4}A_{F}~U_{M18}U_{F18}~G_{L}G_{F}~A_{M}A_{F}~U_{L}A~G_{M}~U_{F18}~U_{L18}~U_{F}~C_{M4}~A_{F}~G_{M}
P-U_{M}~G_{F}~A_{M}A_{L}~C_{M4}A_{F}~U_{M18}U_{F18}~G_{L}G_{F}~A_{M}A_{F}~U_{L18}A~G_{M}~U_{F18}~U_{L18}~U_{F18}~C_{M4}~A_{F}~G_{M}
P-U_{M}~G_{F}~A_{M}A_{L2}~C_{M4}A_{F}~U_{M3}U_{F3}~G_{L}G_{F}~A_{M}A_{F}~U_{L}A~G_{M}~U_{F3}~U_{L3}~U_{F}~C_{M4}~A_{F}~G_{M}
P-U_{M}~G_{F}~A_{M}A_{L2}~C_{M4}A_{F}~U_{M3}U_{F3}~G_{L}G_{F}~A_{M}A_{F}~U_{L3}A~G_{M}~U_{F3}~U_{L3}~U_{F3}~C_{M4}~A_{F}~G_{M}
P-U_{M}~G_{F}~A_{M}A_{L2}~C_{M}A_{F}~U_{M3}U_{F3}~G_{L}G_{F}~A_{M}A_{F}~U_{L}A~G_{M}~U_{F3}~U_{L3}~U_{F3}~C_{M}~A_{F}~G_{M}
P-U_{M}~G_{F}~A_{M}A_{L2}~C_{M}A_{F}~U_{M3}U_{F3}~G_{L}G_{F}~A_{M}A_{F}~U_{L3}A~G_{M}~U_{F3}~U_{L3}~U_{F3}~C_{M}~A_{F}~G_{M}
P-U_{M}~G_{F}~A_{M}A_{L2}~C_{M4}A_{F}~U_{M12}U_{F12}~G_{L}G_{F}~A_{M}A_{F}~U_{L}A~G_{M}~U_{F12}~U_{L12}~U_{F3}~C_{M4}~A_{F}~G_{M}
P-U_{M}~G_{F}~A_{M}A_{L2}~C_{M4}A_{F}~U_{M12}U_{F12}~G_{L}G_{F}~A_{M}A_{F}~U_{L12}A~G_{M}~U_{F12}~U_{L12}~U_{F12}~C_{M4}~A_{F}~G_{M}
P-U_{M}~G_{F}~A_{M}A_{L2}~C_{M}A_{F}~U_{M12}U_{F12}~G_{L}G_{F}~A_{M}A_{F}~U_{L}A~G_{M}~U_{F12}~U_{L12}~U_{F12}~C_{M}~A_{F}~G_{M}
P-U_{M}~G_{F}~A_{M}A_{L2}~C_{M}A_{F}~U_{M12}U_{F12}~G_{L}G_{F}~A_{M}A_{F}~U_{L12}A~G_{M}~U_{F12}~U_{L12}~U_{F12}~C_{M}~A_{F}~G_{M}
P-U_{M}~G_{F}~A_{M}A_{L2}~C_{M4}A_{F}~U_{M18}U_{F18}~G_{L}G_{F}~A_{M}A_{F}~U_{L}A~G_{M}~U_{F18}~U_{L18}~U_{F18}~C_{M4}~A_{F}~G_{M}
P-U_{M}~G_{F}~A_{M}A_{L2}~C_{M4}A_{F}~U_{M18}U_{F18}~G_{L}G_{F}~A_{M}A_{F}~U_{L18}A~G_{M}~U_{F18}~U_{L18}~U_{F18}~C_{M4}~A_{F}~G_{M}
P-U_{M}~G_{F}~A_{M}A_{L2}~C_{M}A_{F}~U_{M18}U_{F18}~G_{L}G_{F}~A_{M}A_{F}~U_{L}A~G_{M}~U_{F18}~U_{L18}~U_{F18}~C_{M}~A_{F}~G_{M}
P-U_{M}~G_{F}~A_{M}A_{L2}~C_{M}A_{F}~U_{M18}U_{F18}~G_{L}G_{F}~A_{M}A_{F}~U_{L18}A~G_{M}~U_{F18}~U_{L18}~U_{F18}~C_{M}~A_{F}~G_{M}
P-U_{M}~G_{F}~A_{L}A_{F}~C_{M}A_{L}~U_{M}U_{F}~G_{M}G_{L}~A_{M}A_{F}~U_{M}A_{F}~G_{L}~U_{F}~U_{M}~U_{L}~C_{M}~A_{F}~G_{M}
P-U_{M}~G_{F}~A_{L}A_{F}~C_{M}A_{L}~U_{M}U_{F}~G_{M}G_{L}~A_{M}A_{F}~U_{M}A~G_{L}~U_{F}~U_{M}~U_{L}~C_{M}~A_{F}~G_{M}
P-U_{M}~G_{F}~A_{L}A_{F}~C_{M4}A_{L}~U_{M3}U_{F3}~G_{M}G_{L}~A_{M}A_{F}~U_{M}A_{F}~G_{L}~U_{F3}~U_{M3}~U_{L}~C_{M4}~A_{F}~G_{M}
P-U_{M}~G_{F}~A_{L}A_{F}~C_{M4}A_{L}~U_{M3}U_{F3}~G_{M}G_{L}~A_{M}A_{F}~U_{M3}A_{F}~G_{L}~U_{F3}~U_{M3}~U_{L3}~C_{M4}~A_{F}~G_{M}
P-U_{M}~G_{F}~A_{L}A_{F}~C_{M4}A_{L}~U_{M12}U_{F12}~G_{M}G_{L}~A_{M}A_{F}~U_{M}A_{F}~G_{L}~U_{F12}~U_{M12}~U_{L}~C_{M4}~A_{F}~G_{M}
P-U_{M}~G_{F}~A_{L}A_{F}~C_{M4}A_{L}~U_{M12}U_{F12}~G_{M}G_{L}~A_{M}A_{F}~U_{M12}A_{F}~G_{L}~U_{F12}~U_{M12}~U_{L12}~C_{M4}~A_{F}~G_{M}
P-U_{M}~G_{F}~A_{L}A_{F}~C_{M4}A_{L}~U_{M18}U_{F18}~G_{M}G_{L}~A_{M}A_{F}~U_{M}A_{F}~G_{L}~U_{F18}~U_{M18}~U_{L}~C_{M4}~A_{F}~G_{M}
P-U_{M}~G_{F}~A_{L}A_{F}~C_{M4}A_{L}~U_{M18}U_{F18}~G_{M}G_{L}~A_{M}A_{F}~U_{M18}A_{F}~G_{L}~U_{F18}~U_{M18}~U_{L18}~C_{M4}~A_{F}~G_{M}
P-U_{M}~G_{F}~A_{L}A_{F2}~C_{M4}A_{L}~U_{M3}U_{F3}~G_{M}G_{L}~A_{M}A_{F}~U_{M}A_{F}~G_{L}~U_{F3}~U_{M3}~U_{L}~C_{M4}~A_{F}~G_{M}
P-U_{M}~G_{F}~A_{L}A_{F2}~C_{M4}A_{L}~U_{M3}U_{F3}~G_{M}G_{L}~A_{M}A_{F}~U_{M3}A_{F}~G_{L}~U_{F3}~U_{M3}~U_{L3}~C_{M4}~A_{F}~G_{M}
P-U_{M}~G_{F}~A_{L}A_{F2}~C_{M}A_{L}~U_{M3}U_{F3}~G_{M}G_{L}~A_{M}A_{F}~U_{M}A_{F}~G_{L}~U_{F3}~U_{M3}~U_{L3}~C_{M}~A_{F}~G_{M}
P-U_{M}~G_{F}~A_{L}A_{F2}~C_{M}A_{L}~U_{M3}U_{F3}~G_{M}G_{L}~A_{M}A_{F}~U_{M3}A_{F}~G_{L}~U_{F3}~U_{M3}~U_{L3}~C_{M}~A_{F}~G_{M}
P-U_{M}~G_{F}~A_{L}A_{F2}~C_{M4}A_{L}~U_{M12}U_{F12}~G_{M}G_{L}~A_{M}A_{F}~U_{M}A_{F}~G_{L}~U_{F12}~U_{M12}~U_{L3}~C_{M4}~A_{F}~G_{M}
P-U_{M}~G_{F}~A_{L}A_{F2}~C_{M4}A_{L}~U_{M12}U_{F12}~G_{M}G_{L}~A_{M}A_{F}~U_{M12}A_{F}~G_{L}~U_{F12}~U_{M12}~U_{L12}~C_{M4}~A_{F}~G_{M}
P-U_{M}~G_{F}~A_{L}A_{F2}~C_{M}A_{L}~U_{M12}U_{F12}~G_{M}G_{L}~A_{M}A_{F}~U_{M}A_{F}~G_{L}~U_{F12}~U_{M12}~U_{L12}~C_{M}~A_{F}~G_{M}
P-U_{M}~G_{F}~A_{L}A_{F2}~C_{M}A_{L}~U_{M12}U_{F12}~G_{M}G_{L}~A_{M}A_{F}~U_{M12}A_{F}~G_{L}~U_{F12}~U_{M12}~U_{L12}~C_{M}~A_{F}~G_{M}
P-U_{M}~G_{F}~A_{L}A_{F2}~C_{M4}A_{L}~U_{M18}U_{F18}~G_{M}G_{L}~A_{M}A_{F}~U_{M}A_{F}~G_{L}~U_{F18}~U_{M18}~U_{L18}~C_{M4}~A_{F}~G_{M}
P-U_{M}~G_{F}~A_{L}A_{F2}~C_{M4}A_{L}~U_{M18}U_{F18}~G_{M}G_{L}~A_{M}A_{F}~U_{M18}A_{F}~G_{L}~U_{F18}~U_{M18}~U_{L18}~C_{M4}~A_{F}~G_{M}
P-U_{M}~G_{F}~A_{L}A_{F2}~C_{M}A_{L}~U_{M18}U_{F18}~G_{M}G_{L}~A_{M}A_{F}~U_{M}A_{F}~G_{L}~U_{F18}~U_{M18}~U_{L18}~C_{M}~A_{F}~G_{M}
P-U_{M}~G_{F}~A_{L}A_{F2}~C_{M}A_{L}~U_{M18}U_{F18}~G_{M}G_{L}~A_{M}A_{F}~U_{M18}A_{F}~G_{L}~U_{F18}~U_{M18}~U_{L18}~C_{M}~A_{F}~G_{M}
P-U_{M}~G_{F}~A_{L}A_{F}~C_{M4}A_{L}~U_{M3}U_{F3}~G_{M}G_{L}~A_{M}A_{F}~U_{M}A~G_{L}~U_{F3}~U_{M3}~U_{L}~C_{M4}~A_{F}~G_{M}
P-U_{M}~G_{F}~A_{L}A_{F}~C_{M4}A_{L}~U_{M3}U_{F3}~G_{M}G_{L}~A_{M}A_{F}~U_{M3}A~G_{L}~U_{F3}~U_{M3}~U_{L3}~C_{M4}~A_{F}~G_{M}
P-U_{M}~G_{F}~A_{L}A_{F}~C_{M4}A_{L}~U_{M12}U_{F12}~G_{M}G_{L}~A_{M}A_{F}~U_{M}A~G_{L}~U_{F12}~U_{M12}~U_{L}~C_{M4}~A_{F}~G_{M}
P-U_{M}~G_{F}~A_{L}A_{F}~C_{M4}A_{L}~U_{M12}U_{F12}~G_{M}G_{L}~A_{M}A_{F}~U_{M12}A~G_{L}~U_{F12}~U_{M12}~U_{L12}~C_{M4}~A_{F}~G_{M}
P-U_{M}~G_{F}~A_{L}A_{F}~C_{M4}A_{L}~U_{M18}U_{P18}~G_{M}G_{L}~A_{M}A_{F}~U_{M}A~G_{L}~U_{F18}~U_{M18}~U_{L}~C_{M4}~A_{F}~G_{M}
P-U_{M}~G_{F}~A_{L}A_{F}~C_{M4}A_{L}~U_{M18}U_{F18}~G_{M}G_{L}~A_{M}A_{F}~U_{M18}A~G_{L}~U_{F18}~U_{M18}~U_{L18}~C_{M4}~A_{F}~G_{M}
P-U_{M}~G_{F}~A_{L}A_{F2}~C_{M4}A_{L}~U_{M3}U_{F3}~G_{M}G_{L}~A_{M}A_{F}~U_{M}A~G_{L}~U_{F3}~U_{M3}~U_{L}~C_{M4}~A_{F}~G_{M}
P-U_{M}~G_{F}~A_{L}A_{F2}~C_{M4}A_{L}~U_{M3}U_{F3}~G_{M}G_{L}~A_{M}A_{F}~U_{M3}A~G_{L}~U_{F3}~U_{M3}~U_{L3}~C_{M4}~A_{F}~G_{M}
P-U_{M}~G_{F}~A_{L}A_{F2}~C_{M}A_{L}~U_{M3}U_{F3}~G_{M}G_{L}~A_{M}A_{F}~U_{M}A~G_{L}~U_{F3}~U_{M3}~U_{L3}~C_{M}~A_{F}~G_{M}
P-U_{M}~G_{F}~A_{L}A_{F2}~C_{M}A_{L}~U_{M3}U_{F3}~G_{M}G_{L}~A_{M}A_{F}~U_{M3}A~G_{L}~U_{F3}~U_{M3}~U_{L3}~C_{M}~A_{F}~G_{M}
P-U_{M}~G_{F}~A_{L}A_{F2}~C_{M4}A_{L}~U_{M12}U_{P12}~G_{M}G_{L}~A_{M}A_{F}~U_{M}A~G_{L}~U_{F12}~U_{M12}~U_{L3}~C_{M4}~A_{F}~G_{M}
P-U_{M}~G_{F}~A_{L}A_{F2}~C_{M4}A_{L}~U_{M12}U_{F12}~G_{M}G_{L}~A_{M}A_{F}~U_{M12}A~G_{L}~U_{F12}~U_{M12}~U_{L12}~C_{M4}~A_{F}~G_{M}
P-U_{M}~G_{F}~A_{L}A_{F2}~C_{M}A_{L}~U_{M12}U_{F12}~G_{M}G_{L}~A_{M}A_{F}~U_{M}A~G_{L}~U_{F12}~U_{M12}~U_{L12}~C_{M}~A_{F}~G_{M}
P-U_{M}~G_{F}~A_{L}A_{F2}~C_{M}A_{L}~U_{M12}U_{F12}~G_{M}G_{L}~A_{M}A_{F}~U_{M12}A~G_{L}~U_{F12}~U_{M12}~U_{L12}~C_{M}~A_{F}~G_{M}
P-U_{M}~G_{F}~A_{L}A_{F2}~C_{M4}A_{L}~U_{M18}U_{P18}~G_{M}G_{L}~A_{M}A_{F}~U_{M}A~G_{L}~U_{F18}~U_{M18}~U_{L18}~C_{M4}~A_{F}~G_{M}
P-U_{M}~G_{F}~A_{L}A_{F2}~C_{M4}A_{L}~U_{M18}U_{P18}~G_{M}G_{L}~A_{M}A_{F}~U_{M18}A~G_{L}~U_{F18}~U_{M18}~U_{L18}~C_{M4}~A_{F}~G_{M}
P-U_{M}~G_{F}~A₁A_{F2}~C_{M}A_{L}~U_{M18}U_{F18}~G_{M}G_{L}~A_{M}A_{F}~U_{M}A~G_{L}~U_{F18}~U_{M18}~U_{L18}~C_{M}~A_{F}~G_{M}
P-U_{M}~G_{F}~A_{L}A_{F2}~C_{M}A_{L}~U_{M18}U_{P18}~G_{M}G_{L}~A_{M}A_{F}~U_{M18}A~G_{L}~U_{F18}~U_{M18}~U_{L18}~C_{M}~A_{F}~G_{M}

### Table 10

### Examples of Murine/Human PTEN ags-siRNA Compositions

### A. Murine/Human PTEN Antisense Sequence:

UUAUCUAUAAUGAUCAGGU

Lima et al. (Cell 150: 883-94, 2012) used this sequence to generate ssRNAi compounds against human/murine PTEN including the following:

5′-VP-Tₘₒₑ~U_{F}~A_{M}U_{F}~C_{M}U_{F}~A_{M}U_{F}~A_{M}A_{F}~
U_{M}G_{F}~A_{M}U_{F}~C_{M}~A_{F}~G_{M}~G_{F}~U_{M}~Aₘₒₑ~Aₘₒₑ

B. Examples of Application of AGSD Rules to these Compounds:

Any of the 5′-end nucleosides (P-U_{M}) shown in the examples of agsRNAi compounds in Table 10 can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 10 with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the compounds in Table 10 can any of those units and linkages provided for herein for overhang precursors.

B. Examples of agsRNAi Compounds:

Compositions of Matter Suitable for Administration to a Subject
with or without a Protective Carrier and for Any Other use as
Provided for Herein (5′-3′)

P-U_{M}~U_{F}~A_{M}U_{F3}~C_{M}U_{F3}~A_{M}U_{F3}~A_{M}A_{F}~U_{M}G_{F}~A_{M}U_{F}~C_{M4}~A_{F}~G_{M}~G_{F}~U_{M3}~A_{F}~G_{M}
P-U_{M}~U_{F3}~A_{M}U_{F3}~C_{M}U_{F}~A_{M}U_{F3}~A_{M}A_{F}~U_{M}G_{F}~A_{M}U_{F}~C_{M4}~A_{F}~G_{M}~G_{F}~U_{M3}~A_{F}~G_{M}
P-U_{M}~U_{F12}~A_{M}U_{P12}~C_{M}U_{F12}~A_{M}U_{P}~A_{M}A_{F}~U_{M}G_{F}~A_{M}U_{F}~C_{M4}~A_{F}~G_{M}~G_{F}~U_{M12}~A_{F}~G_{M}
P-U_{M}~U_{F12}~A_{M}U_{F12}~C_{M}U_{F12}~A_{M}U_{F12}~A_{M}A_{F}~U_{M}G_{F}~A_{M}U_{F}~C_{M}~A_{F}~G_{M}~G_{F}~U_{M12}~A_{F}~G_{M}
P-U_{M}~U_{F18}~A_{M}U_{P18}~C_{M4}U_{F18}~A_{M}U_{P18}~A_{M}A_{F}~U_{M}G_{F}~A_{M}U_{F}~C_{M4}~A_{F}~G_{M}~G_{F}~U_{M}~A_{F}~G_{M}
P-U_{M}~U_{F}~A_{M}U_{F18}~C_{M4}U_{F18}~A_{M}U_{F18}~A_{M}A_{F}~U_{M}G_{F}~A_{M}U_{F}~C_{M4}~A_{F}~G_{M}~G_{F}~U_{M}~A_{F}~G_{M}
P-U_{M}~U_{F3}~A_{M}U_{F3}~C_{M}U_{F3}~A_{M}U_{F3}~A_{M}A_{F}~U_{M}G_{F}~A_{M}U_{F}~C_{M4}~A_{F}~G_{M}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M}G_{F}~A_{M}U_{F3}~C_{M4}~A_{F}~G_{M}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M3}G_{F}~A_{M}U_{F3}~C_{M4}~A_{F}~G_{M}~G_{F}~U_{M3}~A_{F}~G_{M}
P-U_{M}~U_{F12}~A_{M}U_{P12}~C_{M}U_{F12}~A_{M}U_{P12}~A_{M}A_{F}~U_{M}G_{F}~A_{M}U_{F}~C_{M4}~A_{F}~G_{M}~G_{F}~U_{M12}~A_{F}~G_{M}
P-U_{M}~U_{F12}~A_{M}U_{F12}~C_{M4}U_{F12}~A_{M}U_{F12}~A_{M}A_{F}~U_{M}G_{F}~A_{M}U_{F12}~C_{M4}~A_{F}~G_{M}~G_{F}~U_{M12}~A_{F}~G_{M}
P-U_{M}~U_{F12}~A_{M}U_{P12}~C_{M4}U_{F12}~A_{M}U_{P12}~A_{M}A_{F}~U_{M12}G_{P}~A_{M}U_{P12}~C_{M4}~A_{F}~G_{M}~G_{F}~U_{M12}~A_{F}~G_{M}
P-U_{M}~U_{F18}~A_{M}U_{F18}~C_{M}U_{F18}~A_{M}U_{F18}~A_{M}A_{F}~U_{M}G_{F}~A_{M}U_{F}~C_{M4}~A_{F}~G_{M}~G_{F}~U_{M18}~A_{F}~G_{M}
P-U_{M}~U_{F18}~A_{M}U_{P18}~C_{M4}U_{F18}~A_{M}U_{P18}~A_{M}A_{F}~U_{M}G_{F}~A_{M}U_{P18}~C_{M4}~A_{F}~G_{M}~G_{F}~U_{M18}~A_{F}~G_{M}
P-U_{M}~U_{F18}~A_{M}U_{F18}~C_{M4}U_{F18}~A_{M}U_{F18}~A_{M}A_{F}~U_{M1S}G_{F}~A_{M}U_{F18}~C_{M4}~A_{F}~G_{M}~G_{F}~U_{M18}~A_{F}~G_{M}
P-U_{M}~U_{F}~A_{M}U_{F3}~C_{M}U_{F3}~A_{M}U_{F3}~A_{M}A_{F}~U_{M}G_{F}~A_{M}U~C_{M4}~A_{F}~G_{M}~G_{F}~U_{M3}~A_{F}~G_{M}
P-U_{M}~U_{F3}~A_{M}U_{F3}~C_{M}U_{F}~A_{M}U_{F3}~A_{M}A_{F}~U_{M}G_{F}~A_{M}U~C_{M4}~A_{F}~G_{M}~G_{F}~U_{M3}~A_{F}~G_{M}
P-U_{M}~U_{F12}~A_{M}U_{P12}~C_{M}U_{F12}~A_{M}U_{P}~A_{M}A_{F}~U_{M}G_{F}~A_{M}U~C_{M4}~A_{F}~G_{M}~G_{F}~U_{M12}~A_{F}~G_{M}
P-U_{M}~U_{F12}~A_{M}U_{F12}~C_{M}U_{F12}~A_{M}U_{F12}~A_{M}A_{F}~U_{M}G_{F}~A_{M}U~C_{M}~A_{F}~G_{M}~G_{F}~U_{M12}~A_{F}~G_{M}
P-U_{M}~U_{F18}~A_{M}U_{P18}~C_{M4}U_{F18}~A_{M}U_{P18}~A_{M}A_{F}~U_{M}G_{F}~A_{M}U~C_{M4}~A_{F}~G_{M}~G_{F}~U_{M}~A_{F}~G_{M}
P-U_{M}~U_{F}~A_{M}U_{F18}~C_{M4}U_{F18}~A_{M}U_{F18}~A_{M}A_{F}~U_{M}G_{F}~A_{M}U~C_{M4}~A_{F}~G_{M}~G_{F}~U_{M}~A_{F}~G_{M}
P-U_{M}~U_{F3}~A_{M}U_{F3}~C_{M}U_{F3}~A_{M}U_{F3}~A_{M}A_{F}~U_{M}G_{F}~A_{M}U~C_{M4}~A_{F}~G_{M}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M}G_{F}~A_{M}U~C_{M4}~A_{F}~G_{M}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M3}G_{F}~A_{M}U~C_{M4}~A_{F}~G_{M}~G_{F}~U_{M3}~A_{F}~G_{M}
P-U_{M}~U_{F12}~A_{M}U_{P12}~C_{M}U_{F12}~A_{M}U_{P12}~A_{M}A_{F}~U_{M}G_{F}~A_{M}U~C_{M4}~A_{F}~G_{M}~G_{F}~U_{M12}~A_{F}~G_{M}
P-U_{M}~U_{F12}~A_{M}U_{F12}~C_{M4}U_{F12}~A_{M}U_{F12}~A_{M}A_{F}~U_{M}G_{F}~A_{M}U~C_{M4}~A_{F}~G_{M}~G_{F}~U_{M12}~A_{F}~G_{M}
P-U_{M}~U_{F12}~A_{M}U_{P12}~C_{M4}U_{F12}~A_{M}U_{P12}~A_{M}A_{F}~U_{M12}G_{P}~A_{M}U~C_{M4}~A_{F}~G_{M}~G_{F}~U_{M12}~A_{F}~G_{M}
P-U_{M}~U_{F18}~A_{M}U_{P18}~C_{M}U_{F18}~A_{M}U_{P18}~A_{M}A_{F}~U_{M}G_{F}~A_{M}U~C_{M4}~A_{F}~G_{M}~G_{F}~U_{M18}~A_{F}~G_{M}
P-U_{M}~U_{F18}~A_{M}U_{F18}~C_{M4}U_{F18}~A_{M}U_{F18}~A_{M}A_{F}~U_{M}G_{F}~A_{M}U~C_{M4}~A_{F}~G_{M}~G_{F}~U_{M18}~A_{F}~G_{M}
P-U_{M}~U_{F18}~A_{M}U_{P18}~C_{M4}U_{F18}~A_{M}U_{P18}~A_{M}A_{F}~U_{M1S}G_{P}~A_{M}U~C_{M4}~A_{F}~G_{M}~G_{F}~U_{M18}~A_{F}~G_{M}
P-U_{M}~U_{M}~A_{F}U_{M3}~C_{F}U_{M3}~A_{F}U_{M3}~A_{F}A_{F}~U_{M}G_{F}~A_{M}U_{F}~C_{M4}~A_{F}~G_{M}~G_{F}~U_{M3}~A_{F}~G_{M}
P-U_{M}~U_{M3}~A_{F}U_{M3}~C_{F}U_{M}~A_{F}U_{M3}~A_{F}A_{F}~U_{M}G_{F}~A_{M}U_{F}~C_{M4}~A_{F}~G_{M}~G_{F}~U_{M3}~A_{F}~G_{M}
P-U_{M}~U_{M12}~A_{F}U_{M12}~C_{F}U_{M12}~A_{F}U_{M}~A_{F}A_{F}~U_{M}G_{F}~A_{M}U_{F}~C_{M4}~A_{F}~G_{M}~G_{F}~U_{M12}~A_{F}~G_{M}
P-U_{M}~U_{M12}~A_{F}U_{M12}~C_{F}U_{M12}~A_{F}U_{M12}~A_{P}A_{F}~U_{M}G_{F}~A_{M}U_{F}~C_{M}~A_{F}~G_{M}~G_{F}~U_{M12}~A_{F}~G_{M}
P-U_{M}~U_{M18}~A_{F}U_{M18}~C_{F4}U_{M18}~A_{F}U_{M18}~A_{P}A_{F}~U_{M}G_{F}~A_{M}U_{F}~C_{M4}~A_{F}~G_{M}~G_{F}~U_{M}~A_{F}~G_{M}
P-U_{M}~U_{M}~A_{F}U_{M18}~C_{F4}U_{M18}~A_{F}U_{M18}~A_{F}A_{F}~U_{M}G_{F}~A_{M}U_{F}~C_{M4}~A_{F}~G_{M}~G_{F}~U_{M}~A_{F}~G_{M}
P-U_{M}~U_{M}~A_{F}U_{M3}~C_{F}U_{M3}~A_{F}U_{M3}~A_{F}A_{F}~U_{M}G_{F}~A_{M}U_{F}~C_{M4}~A_{F}~G_{M}~G_{F}~U_{M3}~A_{F}~G_{M}
P-U_{M}~U_{M}~A_{F}U_{M3}~C_{F4}U_{M3}~A_{F}U_{M3}~A_{F}A_{F}~U_{M}G_{F}~A_{M}U_{F3}~C_{M4}~A_{F}~G_{M}~G_{F}~U_{M3}~A_{F}~G_{M}
P-U_{M}~U_{M}~A_{F}U_{M3}~C_{F4}U_{M3}~A_{F}U_{M3}~A_{F}A_{F}~U_{M3}G_{F}~A_{M}U_{F3}~C_{M4}~A_{F}~G_{M}~G_{F}~U_{M3}~A_{F}~G_{M}
P-U_{M}~U_{M}~A_{F}U_{M12}~C_{F}U_{M12}~A_{F}U_{M12}~A_{F}A_{F}~U_{M}G_{F}~A_{M}U_{F}~C_{M4}~A_{F}~G_{M}~G_{F}~U_{M12}~A_{F}~G_{M}
P-U_{M}~U_{M}~A_{F}U_{M12}~C_{F4}U_{M12}~A_{F}U_{M12}~A_{F}A_{F}~U_{M}G_{F}~A_{M}U_{F12}~C_{M4}~A_{F}~G_{M}~G_{F}~U_{M12}~A_{F}~G_{M}
P-U_{M}~U_{M}~A_{F}U_{M12}~C_{F4}U_{M12}~A_{F}U_{M12}~A_{F}A_{F}~U_{M12}G_{F}~A_{M}U_{F12}~C_{M4}~A_{F}~G_{M}~G_{F}~U_{M12}~A_{F}~G_{M}
P-U_{M}~U_{M}~A_{F}U_{M18}~C_{F}U_{M18}~A_{F}U_{M18}~A_{F}A_{F}~U_{M}G_{F}~A_{M}U_{F}~C_{M4}~A_{F}~G_{M}~G_{F}~U_{M18}~A_{F}~G_{M}
P-U_{M}~U_{M}~A_{F}U_{M18}~C_{F4}U_{M18}~A_{F}U_{M18}~A_{F}A_{F}~U_{M}G_{F}~A_{M}U_{F18}~C_{M4}~A_{F}~G_{M}~G_{F}~U_{M18}~A_{F}~G_{M}
P-U_{M}~U_{M}~A_{F}U_{M18}~C_{F4}U_{M18}~A_{F}U_{M18}~A_{F}A_{F}~U_{M18}G_{F}~A_{M}U_{F18}~C_{M4}~A_{F}~G_{M}~G_{F}~U_{M18}~A_{F}~G_{M}
P-U_{M}~U_{F}~A_{L}U_{F}~C_{M}U_{L}~A_{M}U_{F}~A_{L}A_{F}~U_{M}G_{F}~A_{L}U_{F}~C_{L}~A_{F}~G_{M}~G_{L}~U_{M}~A_{F}~G_{M}
P-U_{M}~U_{F}~A_{L}U_{F}~C_{M}U_{L}~A_{M}U_{F}~A_{L}A_{F}~U_{M}G_{F}~A_{L}U~C_{L}~A_{F}~G_{M}~G_{L}~U_{M}~A_{F}~G_{M}
P-U_{M}~U_{M}~A_{L}U_{M}~C_{F}U_{L}~A_{F}U_{M}~A_{L}A_{F}~U_{M}G_{F}~A_{L}U_{F}~C_{L}~A_{F}~G_{M}~G_{L}~U_{M}~A_{F}~G_{M}
P-U_{M}~U_{F}~A_{L}U_{F3}~C_{M}U_{L3}~A_{M}U_{F3}~A_{L}A_{F}~U_{M}G_{F}~A_{L}U_{F}~C_{L4}~A_{F}~G_{M}~G_{L}~U_{M3}~A_{F}~G_{M}
P-U_{M}~U_{F3}~A_{L}U_{F3}~C_{M}U_{L}~A_{M}U_{F3}~A_{L}A_{F}~U_{M}G_{F}~A_{L}U_{F}~C_{L4}~A_{F}~G_{M}~G_{L}~U_{M3}~A_{F}~G_{M}
P-U_{M}~U_{F12}~A_{L}U_{F12}~C_{M}U_{L12}~A_{M}U_{F}~A_{L}A_{F}~U_{M}G_{F}~A_{L}U_{F}~C_{L4}~A_{F}~G_{M}~G_{L}~U_{M12}~A_{F}~G_{M}
P-U_{M}~U_{F12}~A_{L}U_{F12}~C_{M}U_{L12}~A_{M}U_{F12}~A_{L}A_{F}~U_{M}G_{F}~A_{L}U_{F}~C_{L}~A_{F}~G_{M}~G_{L}~U_{M12}~A_{F}~G_{M}
P-U_{M}~U_{F18}~A_{L}U_{F18}~C_{M4}U_{L18}~A_{M}U_{F18}~A_{L}A_{F}~U_{M}G_{F}~A_{L}U_{F}~C_{L4}~A_{F}~G_{M}~G_{L}~U_{M}~A_{F}~G_{M}
P-U_{M}~U_{F}~A_{L}U_{F18}~C_{M4}U_{L18}~A_{M}U_{F18}~A_{L}A_{F}~U_{M}G_{F}~A_{L}U_{F}~C_{L4}~A_{F}~G_{M}~G_{L}~U_{M}~A_{F}~G_{M}
P-U_{M}~U_{F3}~A_{L}U_{F3}~C_{M}U_{L3}~A_{M}U_{F3}~A_{L}A_{F}~U_{M}G_{F}~A_{L}U_{F}~C_{L4}~A_{F}~G_{M}~G_{L}~U_{M3}~A_{F}~G_{M}
P-U_{M}~U_{F3}~A_{L}U_{F3}~C_{M4}U_{L3}~A_{M}U_{F3}~A_{L}A_{F}~U_{M}G_{F}~A_{L}U_{F3}~C_{L4}~A_{F}~G_{M}~G_{L}~U_{M3}~A_{F}~G_{M}
P-U_{M}~U_{F3}~A_{L}U_{F3}~C_{M4}U_{L3}~A_{M}U_{F3}~A_{L}A_{F}~U_{M3}G_{F}~A_{L}U_{F3}~C_{L4}~A_{F}~G_{M}~G_{L}~U_{M3}~A_{F}~G_{M}
P-U_{M}~U_{F12}~A_{L}U_{F12}~C_{M}U_{L12}~A_{M}U_{F12}~A_{L}A_{F}~U_{M}G_{F}~A_{L}U_{F}~C_{L4}~A_{F}~G_{M}~G_{L}~U_{M12}~A_{F}~G_{M}
P-U_{M}~U_{F12}~A_{L}U_{F12}~C_{M4}U_{L12}~A_{M}U_{F12}~A_{L}A_{F}~U_{M}G_{F}~A_{L}U_{F12}~C_{L4}~A_{F}~G_{M}~G_{L}~U_{M12}~A_{F}~G_{M}
P-U_{M}~U_{F12}~A_{L}U_{F12}~C_{M4}U_{L12}~A_{M}U_{F12}~A_{L}A_{F}~U_{M12}G_{F}~A_{L}U_{F12}~C_{L4}~A_{F}~G_{M}~G_{L}~U_{M12}~A_{F}~G_{M}
P-U_{M}~U_{F18}~A_{L}U_{F18}~C_{M}U_{L18}~A_{M}U_{F18}~A_{L}A_{F}~U_{M}G_{F}~A_{L}U_{F}~C_{L4}~A_{F}~G_{M}~G_{L}~U_{M18}~A_{F}~G_{M}
P-U_{M}~U_{F18}~A_{L}U_{F18}~C_{M4}U_{L18}~A_{M}U_{F18}~A_{L}A_{F}~U_{M}G_{F}~A_{L}U_{F18}~C_{L4}~A_{F}~G_{M}~G_{L}~U_{M18}~A_{F}~G_{M}
P-U_{M}~U_{F18}~A_{L}U_{F18}~C_{M4}U_{L18}~A_{M}U_{F18}~A_{L}A_{F}~U_{M18}G_{F}~A_{L}U_{F18}~C_{L4}~A_{F}~G_{M}~G_{L}~U_{M18}~A_{F}~G_{M}
P-U_{M}~U_{F}~A_{L}U_{F3}~C_{M}U_{L3}~A_{M}U_{F3}~A_{L}A_{F}~U_{M}G_{F}~A_{L}U~C_{L4}~A_{F}~G_{M}~G_{L}~U_{M3}~A_{F}~G_{M}
P-U_{M}~U_{F3}~A_{L}U_{F3}~C_{M}U_{L}~A_{M}U_{F3}~A_{L}A_{F}~U_{M}G_{F}~A_{L}U~C_{L4}~A_{F}~G_{M}~G_{L}~U_{M3}~A_{F}~G_{M}
P-U_{M}~U_{F12}~A_{L}U_{F12}~C_{M}U_{L12}~A_{M}U_{F}~A_{L}A_{F}~U_{M}G_{F}~A_{L}U~C_{L4}~A_{F}~G_{M}~G_{L}~U_{M12}~A_{F}~G_{M}
P-U_{M}~U_{F12}~A_{L}U_{F12}~C_{M}U_{L12}~A_{M}U_{F12}~A_{L}A_{F}~U_{M}G_{F}~A_{L}U~C_{L}~A_{F}~G_{M}~G_{L}~U_{M12}~A_{F}~G_{M}
P-U_{M}~U_{F18}~A_{L}U_{F18}~C_{M4}U_{L18}~A_{M}U_{F18}~A_{L}A_{F}~U_{M}G_{F}~A_{L}U~C_{L4}~A_{F}~G_{M}~G_{L}~U_{M}~A_{F}~G_{M}
P-U_{M}~U_{F}~A_{L}U_{F18}~C_{M4}U_{L18}~A_{M}U_{F18}~A_{L}A_{F}~U_{M}G_{F}~A_{L}U~C_{L4}~A_{F}~G_{M}~G_{L}~U_{M}~A_{F}~G_{M}
P-U_{M}~U_{F3}~A_{L}U_{F3}~C_{M}U_{L3}~A_{M}U_{F3}~A_{L}A_{F}~U_{M}G_{F}~A_{L}U~C_{L4}~A_{F}~G_{M}~G_{L}~U_{M3}~A_{F}~G_{M}
P-U_{M}~U_{F3}~A_{L}U_{F3}~C_{M4}U_{L3}~A_{M}U_{F3}~A_{L}A_{F}~U_{M}G_{F}~A_{L}U~C_{L4}~A_{F}~G_{M}~G_{L}~U_{M3}~A_{F}~G_{M}
P-U_{M}~U_{F3}~A_{L}U_{F3}~C_{M4}U_{L3}~A_{M}U_{F3}~A_{L}A_{F}~U_{M3}G_{F}~A_{L}U~C_{L4}~A_{F}~G_{M}~G_{L}~U_{M3}~A_{F}~G_{M}
P-U_{M}~U_{F12}~A_{L}U_{F12}~C_{M}U_{L12}~A_{M}U_{F12}~A_{L}A_{F}~U_{M}G_{F}~A_{L}U~C_{L4}~A_{F}~G_{M}~G_{L}~U_{M12}~A_{F}~G_{M}
P-U_{M}~U_{F12}~A_{L}U_{F12}~C_{M4}U_{L12}~A_{M}U_{F12}~A_{L}A_{F}~U_{M}G_{F}~A_{L}U~C_{L4}~A_{F}~G_{M}~G_{L}~U_{M12}~A_{F}~G_{M}
P-U_{M}~U_{F12}~A_{L}U_{F12}~C_{M4}U_{L12}~A_{M}U_{F12}~A_{L}A_{F}~U_{M12}G_{F}~A_{L}U~C_{L4}~A_{F}~G_{M}~G_{L}~U_{M12}~A_{F}~G_{M}
P-U_{M}~U_{F18}~A_{L}U_{F18}~C_{M}U_{L18}~A_{M}U_{F18}~A_{L}A_{F}~U_{M}G_{F}~A_{L}U~C_{L4}~A_{F}~G_{M}~G_{L}~U_{M18}~A_{F}~G_{M}
P-U_{M}~U_{F18}~A_{L}U_{F18}~C_{M4}U_{L18}~A_{M}U_{F18}~A_{L}A_{F}~U_{M}G_{F}~A_{L}U~C_{L4}~A_{F}~G_{M}~G_{L}~U_{M18}~A_{F}~G_{M}
P-U_{M}~U_{F18}~A_{L}U_{F18}~C_{M4}U_{L18}~A_{M}U_{F18}~A_{L}A_{F}~U_{M18}G_{F}~A_{L}U~C_{L4}~A_{F}~G_{M}~G_{L}~U_{M18}~A_{F}~G_{M}
P-U_{M}~U_{M}~A_{L}U_{M3}~C_{F}U_{L3}~A_{F}U_{M3}~A_{L}A_{F}~U_{M}G_{F}~A_{L}U_{F}~C_{L4}~A_{F}~G_{M}~G_{L}~U_{M3}~A_{F}~G_{M}
P-U_{M}~U_{M3}~A_{L}U_{M3}~C_{F}U_{L}~A_{F}U_{M3}~A_{L}A_{F}~U_{M}G_{F}~A_{L}U_{F}~C_{L4}~A_{F}~G_{M}~G_{L}~U_{M3}~A_{F}~G_{M}
P-U_{M}~U_{M12}~A_{L}U_{M12}~C_{F}U_{L12}~A_{F}U_{M}~A_{L}A_{F}~U_{M}G_{F}~A_{L}U_{F}~C_{L4}~A_{F}~G_{M}~G_{L}~U_{M12}~A_{F}~G_{M}
P-U_{M}~U_{M12}~A_{L}U_{M12}~C_{F}U_{L12}~A_{F}U_{M12}~A_{L}A_{F}~U_{M}G_{F}~A_{L}U_{F}~C_{L}~A_{F}~G_{M}~G_{L}~U_{M12}~A_{F}~G_{M}
P-U_{M}~U_{M18}~A_{L}U_{M18}~C_{F4}U_{L18}~A_{F}U_{M18}~A_{L}A_{F}~U_{M}G_{F}~A_{L}U_{F}~C_{L4}~A_{F}~G_{M}~G_{L}~U_{M}~A_{F}~G_{M}
P-U_{M}~U_{M}~A_{L}U_{M18}~C_{F4}U_{L18}~A_{F}U_{M18}~A₁A_{F}~U_{M}G_{F}~A_{L}U_{F}~C_{L4}~A_{F}~G_{M}~G_{L}~U_{M}~A_{F}~G_{M}
P-U_{M}~U_{M}~A_{L}U_{M3}~C_{F}U_{L3}~A_{F}U_{M3}~A_{L}A_{F}~U_{M}G_{F}~A_{L}U_{F}~C_{L4}~A_{F}~G_{M}~G_{L}~U_{M3}~A_{F}~G_{M}
P-U_{M}~U_{M}~A_{L}U_{M3}~C_{F4}U_{L3}~A_{F}U_{M3}~A_{L}A_{F}~U_{M}G_{F}~A_{L}U_{F3}~C_{L4}~A_{F}~G_{M}~G_{L}~U_{M3}~A_{F}~G_{M}
P-U_{M}~U_{M}~A_{L}U_{M3}~C_{F4}U_{L3}~A_{F}U_{M3}~A_{L}A_{F}~U_{M3}G_{F}~A_{L}U_{F3}~C_{L4}~A_{F}~G_{M}~G_{L}~U_{M3}~A_{F}~G_{M}
P-U_{M}~U_{M}~A_{L}U_{M12}~C_{F}U_{L12}~A_{F}U_{M12}~A_{L}A_{F}~U_{M}G_{F}~A_{L}U_{F}~C_{L4}~A_{F}~G_{M}~G_{L}~U_{M12}~A_{F}~G_{M}
P-U_{M}~U_{M}~A_{L}U_{M12}~C_{F4}U_{L12}~A_{F}U_{M12}~A_{L}A_{F}~U_{M}G_{F}~A_{L}U_{F12}~C_{L4}~A_{F}~G_{M}~G_{L}~U_{M12}~A_{F}~G_{M}
P-U_{M}~U_{M}~A_{L}U_{M12}~C_{F4}U_{L12}~A_{F}U_{M12}~A_{L}A_{F}~U_{M12}G_{P}~A_{L}U_{P12}~C_{L4}~A_{F}~G_{M}~G_{L}~U_{M12}~A_{F}~G_{M}
P-U_{M}~U_{M}~A_{L}U_{M18}~C_{F}U_{L18}~A_{F}U_{M18}~A_{L}A_{F}~U_{M}G_{F}~A_{L}U_{F}~C_{L4}~A_{F}~G_{M}~G_{L}~U_{M18}~A_{F}~G_{M}
P-U_{M}~U_{M}~A_{L}U_{M18}~C_{F4}U_{L18}~A_{F}U_{M18}~A_{L}A_{F}~U_{M}G_{F}~A_{L}U_{F18}~C_{L4}~A_{F}~G_{M}~G_{L}~U_{M18}~A_{F}~G_{M}
P-U_{M}~U_{M}~A_{L}U_{M18}~C_{F4}U_{L18}~A_{F}U_{M18}~A_{L}A_{F}~U_{M1S}G_{P}~A_{L}U_{P18}~C_{L4}~A_{F}~G_{M}~G_{L}~U_{M18}~A_{F}~G_{M}
P-U_{M}~U_{F}~A_{M}U_{L}~C_{M}U_{F}~A_{L}U_{F}~A_{M}A_{L}~U_{M}G_{F}~A_{M}U_{F}~C_{L}~A_{F}~G_{L}~G_{F}~U_{M}~A_{F}~G_{M}
P-U_{M}~U_{F}~A_{M}U_{L}~C_{M}U_{F}~A_{L}U_{F}~A_{M}A_{L}~U_{M}G_{F}~A_{M}U~C_{L}~A_{F}~G_{L}~G_{F}~U_{M}~A_{F}~G_{M}
P-U_{M}~U_{M}~A_{F}U_{L}~C_{F}U_{M}~A_{L}U_{M}~A_{F}A_{L}~U_{M}G_{F}~A_{M}U_{F}~C_{L}~A_{F}~G_{L}~G_{F}~U_{M}~A_{F}~G_{M}
P-U_{M}~U_{F}~A_{M}U_{L3}~C_{M}U_{F3}~A_{L}U_{F3}~A_{M}A_{L}~U_{M}G_{F}~A_{M}U_{F}~C_{L4}~A_{F}~G_{L}~G_{F}~U_{M3}~A_{F}~G_{M}
P-U_{M}~U_{F3}~A_{M}U_{L3}~C_{M}U_{F}~A_{L}U_{F3}~A_{M}A_{L}~U_{M}G_{F}~A_{M}U_{F}~C_{L4}~A_{F}~G_{L}~G_{F}~U_{M3}~A_{F}~G_{M}
P-U_{M}~U_{F12}~A_{M}U_{L12}~C_{M}U_{F12}~A_{L}U_{F}~A_{M}A_{L}~U_{M}G_{F}~A_{M}U_{F}~C_{L4}~A_{F}~G_{L}~G_{F}~U_{M12}~A_{F}~G_{M}
P-U_{M}~U_{F12}~A_{M}U_{L12}~C_{M}U_{F12}~A_{L}U_{P12}~A_{M}A_{L}~U_{M}G_{F}~A_{M}U_{F}~C_{L}~A_{F}~G_{L}~G_{F}~U_{M12}~A_{F}~G_{M}
P-U_{M}~U_{F18}~A_{M}U_{L18}~C_{M4}U_{F18}~A_{L}U_{F18}~A_{M}A_{L}~U_{M}G_{F}~A_{M}U_{F}~C_{L4}~A_{F}~G_{L}~G_{F}~U_{M}~A_{F}~G_{M}
P-U_{M}~U_{F}~A_{M}U_{L18}~C_{M4}U_{F18}~A_{L}U_{F18}~A_{M}A_{L}~U_{M}G_{F}~A_{M}U_{F}~C_{L4}~A_{F}~G_{L}~G_{F}~U_{M}~A_{F}~G_{M}
P-U_{M}~U_{F3}~A_{M}U_{L3}~C_{M}U_{F3}~A_{L}U_{F3}~A_{M}A_{L}~U_{M}G_{F}~A_{M}U_{F}~C_{L4}~A_{F}~G_{L}~G_{F}~U_{M3}~A_{F}~G_{M}
P-U_{M}~U_{F3}~A_{M}U_{L3}~C_{M4}U_{F3}~A_{L}U_{F3}~A_{M}A_{L}~U_{M}G_{F}~A_{M}U_{F3}~C_{L4}~A_{F}~G_{L}~G_{F}~U_{M3}~A_{F}~G_{M}
P-U_{M}~U_{F3}~A_{M}U_{L3}~C_{M4}U_{F3}~A_{L}U_{F3}~A_{M}A_{L}~U_{M3}G_{F}~A_{M}U_{F3}~C_{L4}~A_{F}~G_{L}~G_{F}~U_{M3}~A_{F}~G_{M}
P-U_{M}~U_{F12}~A_{M}U_{L12}~C_{M}U_{F12}~A_{L}U_{F12}~A_{M}A_{L}~U_{M}G_{F}~A_{M}U_{F}~C_{L4}~A_{F}~G_{L}~G_{F}~U_{M12}~A_{F}~G_{M}
P-U_{M}~U_{F12}~A_{M}U_{L12}~C_{M4}U_{F12}~A_{L}U_{P12}~A_{M}A_{L}~U_{M}G_{F}~A_{M}U_{P12}~C_{L4}~A_{F}~G_{L}~G_{F}~U_{M12}~A_{F}~G_{M}
P-U_{M}~U_{F12}~A_{M}U_{L12}~C_{M4}U_{F12}~A_{L}U_{F12}~A_{M}A_{L}~U_{M12}G_{F}~A_{M}U_{F12}~C_{L4}~A_{F}~G_{L}~G_{F}~U_{M12}~A_{F}~G_{M}
P-U_{M}~U_{F18}~A_{M}U_{L18}~C_{M}U_{F18}~A_{L}U_{F18}~A_{M}A_{L}~U_{M}G_{F}~A_{M}U_{F}~C_{L4}~A_{F}~G_{L}~G_{F}~U_{M18}~A_{F}~G_{M}
P-U_{M}~U_{F18}~A_{M}U_{L18}~C_{M4}U_{F18}~A_{L}U_{P18}~A_{M}A_{L}~U_{M}G_{F}~A_{M}U_{P18}~C_{L4}~A_{F}~G_{L}~G_{F}~U_{M18}~A_{F}~G_{M}
P-U_{M}~U_{F18}~A_{M}U_{L18}~C_{M4}U_{F18}~A_{L}U_{F18}~A_{M}A_{L}~U_{M1S}G_{F}~A_{M}U_{F18}~C_{L4}~A_{F}~G_{L}~G_{F}~U_{M18}~A_{F}~G_{M}
P-U_{M}~U_{F}~A_{M}U_{L3}~C_{M}U_{F3}~A_{L}U_{F3}~A_{M}A_{L}~U_{M}G_{F}~A_{M}U~C_{L4}~A_{F}~G_{L}~G_{F}~U_{M3}~A_{F}~G_{M}
P-U_{M}~U_{F3}~A_{M}U_{L3}~C_{M}U_{F}~A_{L}U_{F3}~A_{M}A_{L}~U_{M}G_{F}~A_{M}U~C_{L4}~A_{F}~G_{L}~G_{F}~U_{M3}~A_{F}~G_{M}
P-U_{M}~U_{F12}~A_{M}U_{L12}~C_{M}U_{F12}~A_{L}U_{F}~A_{M}A_{L}~U_{M}G_{F}~A_{M}U~C_{L4}~A_{F}~G_{L}~G_{F}~U_{M12}~A_{F}~G_{M}
P-U_{M}~U_{F12}~A_{M}U_{L12}~C_{M}U_{F12}~A_{L}U_{F12}~A_{M}A_{L}~U_{M}G_{F}~A_{M}U~C_{L}~A_{F}~G_{L}~G_{F}~U_{M12}~A_{F}~G_{M}
P-U_{M}~U_{F18}~A_{M}U_{L18}~C_{M4}U_{F18}~A_{L}U_{F18}~A_{M}A_{L}~U_{M}G_{F}~A_{M}U~C_{L4}~A_{F}~G_{L}~G_{F}~U_{M}~A_{F}~G_{M}
P-U_{M}~U_{F}~A_{M}U_{L18}~C_{M4}U_{F18}~A_{L}U_{F18}~A_{M}A_{L}~U_{M}G_{F}~A_{M}U~C_{L4}~A_{F}~G_{L}~G_{F}~U_{M}~A_{F}~G_{M}
P-U_{M}~U_{F3}~A_{M}U_{L3}~C_{M}U_{F3}~A_{L}U_{F3}~A_{M}A_{L}~U_{M}G_{F}~A_{M}U~C_{L4}~A_{F}~G_{L}~G_{F}~U_{M3}~A_{F}~G_{M}
P-U_{M}~U_{F3}~A_{M}U_{L3}~C_{M4}U_{F3}~A_{L}U_{F3}~A_{M}A_{L}~U_{M}G_{F}~A_{M}U~C_{L4}~A_{F}~G_{L}~G_{F}~U_{M3}~A_{F}~G_{M}
P-U_{M}~U_{F3}~A_{M}U_{L3}~C_{M4}U_{F3}~A_{L}U_{F3}~A_{M}A_{L}~U_{M3}G_{F}~A_{M}U~C_{L4}~A_{F}~G_{L}~G_{F}~U_{M3}~A_{F}~G_{M}
P-U_{M}~U_{F12}~A_{M}U_{L12}~C_{M}U_{F12}~A_{L}U_{P12}~A_{M}A_{L}~U_{M}G_{F}~A_{M}U~C_{L4}~A_{F}~G_{L}~G_{F}~U_{M12}~A_{F}~G_{M}
P-U_{M}~U_{F12}~A_{M}U_{L12}~C_{M4}U_{F12}~A_{L}U_{F12}~A_{M}A_{L}~U_{M}G_{F}~A_{M}U~C_{L4}~A_{F}~G_{L}~G_{F}~U_{M12}~A_{F}~G_{M}
P-U_{M}~U_{F12}~A_{M}U_{L12}~C_{M4}U_{F12}~A_{L}U_{F12}~A_{M}A_{L}~U_{M12}G_{F}~A_{M}U~C_{L4}~A_{F}~G_{L}~G_{F}~U_{M12}~A_{F}~G_{M}
P-U_{M}~U_{F18}~A_{M}U_{L18}~C_{M}U_{F18}~A_{L}U_{F18}~A_{M}A_{L}~U_{M}G_{F}~A_{M}U~C_{L4}~A_{F}~G_{L}~G_{F}~U_{M18}~A_{F}~G_{M}
P-U_{M}~U_{F18}~A_{M}U_{L18}~C_{M4}U_{F18}~A_{L}U_{F18}~A_{M}A_{L}~U_{M}G_{F}~A_{M}U~C_{L4}~A_{F}~G_{L}~G_{F}~U_{M18}~A_{F}~G_{M}
P-U_{M}~U_{F18}~A_{M}U_{L18}~C_{M4}U_{F18}~A_{L}U_{F18}~A_{M}A_{L}~U_{M1S}G_{F}~A_{M}U~C_{L4}~A_{F}~G_{L}~G_{F}~U_{M18}~A_{F}~G_{M}
P-U_{M}~U_{M}~A_{F}U_{L3}~C_{F}U_{M3}~A_{L}U_{M3}~A_{F}~A_{L}~U_{M}G_{F}~A_{M}U_{F}~C_{L4}~A_{F}~G_{L}~G_{F}~U_{M3}~A_{F}~G_{M}
P-U_{M}~U_{M3}~A_{F}U_{L3}~C_{F}U_{M}~A_{L}U_{M3}~A_{F}A_{L}~U_{M}G_{F}~A_{M}U_{F}~C_{L4}~A_{F}~G_{L}~G_{F}~U_{M3}~A_{F}~G_{M}
P-U_{M}~U_{M12}~A_{F}U_{L12}~C_{F}U_{M12}~A_{L}U_{M}~A_{F}A_{L}~U_{M}G_{F}~A_{M}U_{F}~C_{L4}~A_{F}~G_{L}~G_{F}~U_{M12}~A_{F}~G_{M}
P-U_{M}~U_{M12}~A_{F}U_{L12}~C_{F}U_{M12}~A_{L}U_{M12}~A_{F}A_{L}~U_{M}G_{F}~A_{M}U_{F}~C_{L}~A_{F}~G_{L}~G_{F}~U_{M12}~A_{F}~G_{M}
P-U_{M}~U_{M18}~A_{F}U_{L18}~C_{F4}U_{M18}~A_{L}U_{M18}~A_{F}A_{L}~U_{M}G_{F}~A_{M}U_{F}~C_{L4}~A_{F}~G_{L}~G_{F}~U_{M}~A_{F}~G_{M}
P-U_{M}~U_{M}~A_{F}U_{L18}~C_{F4}U_{M18}~A_{L}U_{M18}~A_{F}A_{L}~U_{M}G_{F}~A_{M}U_{F}~C_{L4}~A_{F}~G_{L}~G_{F}~U_{M}~A_{F}~G_{M}
P-U_{M}~U_{M}~A_{F}U_{L3}~C_{F}U_{M3}~A_{L}U_{M3}~A_{F}A_{L}~U_{M}G_{F}~A_{M}U_{F}~C_{L4}~A_{F}~G_{L}~G_{F}~U_{M3}~A_{F}~G_{M}
P-U_{M}~U_{M}~A_{F}U_{L3}~C_{F4}U_{M3}~A_{L}U_{M3}~A_{F}A_{L}~U_{M}G_{F}~A_{M}U_{F3}~C_{L4}~A_{F}~G_{L}~G_{F}~U_{M3}~A_{F}~G_{M}
P-U_{M}~U_{M}~A_{F}U_{L3}~C_{F4}U_{M3}~A_{L}U_{M3}~A_{F}A_{L}~U_{M}G_{F}~A_{M}U_{F3}~C_{L4}~A_{F}~G_{L}~G_{F}~U_{M3}~A_{F}~G_{M}
P-U_{M}~U_{M}~A_{F}U_{L12}~C_{F}U_{M12}~A_{L}U_{M12}~A_{F}A_{L}~U_{M}G_{F}~A_{M}U_{F}~C_{L4}~A_{F}~G_{L}~G_{F}~U_{M12}~A_{F}~G_{M}
P-U_{M}~U_{M}~A_{F}U_{L12}~C_{F4}U_{M12}~A_{L}U_{M12}~A_{F}A_{L}~U_{M}G_{F}~A_{M}U_{F12}~C_{L4}~A_{F}~G_{L}~G_{F}~U_{M12}~A_{F}~G_{M}
P-U_{M}~U_{M}~A_{F}U_{L12}~C_{F4}U_{M12}~A_{L}U_{M12}~A_{F}A_{L}~U_{M12}G_{F}~A_{M}U_{F12}~C_{L4}~A_{F}~G_{L}~G_{F}~U_{M12}~A_{F}~G_{M}
P-U_{M}~U_{M}~A_{F}U_{L18}~C_{F}U_{M18}~A_{L}U_{M18}~A_{F}A_{L}~U_{M}G_{F}~A_{M}U_{F}~C_{L4}~A_{F}~G_{L}~G_{F}~U_{M18}~A_{F}~G_{M}
P-U_{M}~U_{M}~A_{F}U_{L18}~C_{F4}U_{M18}~A_{L}U_{M18}~A_{F}A_{L}~U_{M}G_{F}~A_{M}U_{F18}~C_{L4}~A_{F}~G_{L}~G_{F}~U_{M18}~A_{F}~G_{M}
P-U_{M}~U_{M}~A_{F}U_{L18}~C_{F4}U_{M18}~A_{L}U_{M18}~A_{F}A_{L}~U_{M1S}G_{F}~A_{M}U_{F18}~C_{L4}~A_{F}~G_{L}~G_{F}~U_{M18}~A_{F}~G_{M}
P-U_{M}~U_{F}~A_{L}U_{F}~C_{M}U_{F}~A_{M}U_{L}~A_{M}A_{F}~U_{L}G_{F}~A_{M}U_{F}~C_{M}~A_{F}~G_{L}~G_{F}~U_{M}~A_{F}~G_{M}
P-U_{M}~U_{F}~A_{L}U_{F}~C_{M}U_{F}~A_{M}U_{L}~A_{M}A_{F}~U_{L}G_{F}~A_{M}U~C_{M}~A_{F}~G_{L}~G_{F}~U_{M}~A_{F}~G_{M}
P-U_{M}~U_{M}~A_{L}U_{M}~C_{F}U_{M}~A_{F}U_{L}~A_{F}A_{F}~U_{L}G_{F}~A_{M}U_{F}~C_{M}~A_{F}~G_{L}~G_{F}~U_{M}~A_{F}~G_{M}
P-U_{M}~U_{F}~A_{L}U_{F3}~C_{M}U_{F3}~A_{M}U_{L3}~A_{M}A_{F}~U_{L}G_{F}~A_{M}U_{F}~C_{M4}~A_{F}~G_{L}~G_{F}~U_{M3}~A_{F}~G_{M}
P-U_{M}~U_{F3}~A_{L}U_{F3}~C_{M}U_{F}~A_{M}U_{L3}~A_{M}A_{F}~U_{L}G_{F}~A_{M}U_{F}~C_{M4}~A_{F}~G_{L}~G_{F}~U_{M3}~A_{F}~G_{M}
P-U_{M}~U_{F12}~A_{L}U_{F12}~C_{M}U_{F12}~A_{M}U_{L}~A_{M}A_{F}~U_{L}G_{F}~A_{M}U_{F}~C_{M4}~A_{F}~G_{L}~G_{F}~U_{M12}~A_{F}~G_{M}
P-U_{M}~U_{F12}~A_{L}U_{F12}~C_{M}U_{F12}~A_{M}U_{L12}~A_{M}A_{F}~U_{L}G_{F}~A_{M}U_{F}~C_{M}~A_{F}~G_{L}~G_{F}~U_{M12}~A_{F}~G_{M}
P-U_{M}~U_{F18}~A_{L}U_{F18}~C_{M4}U_{F18}~A_{M}U_{L18}~A_{M}A_{F}~U_{L}G_{F}~A_{M}U_{F}~C_{M4}~A_{F}~G_{L}~G_{F}~U_{M}~A_{F}~G_{M}
P-U_{M}~U_{F}~A_{L}U_{F18}~C_{M4}U_{F18}~A_{M}U_{L18}~A_{M}A_{F}~U_{L}G_{F}~A_{M}U_{F}~C_{M4}~A_{F}~G_{L}~G_{F}~U_{M}~A_{F}~G_{M}
P-U_{M}~U_{F3}~A_{L}U_{F3}~C_{M}U_{F3}~A_{M}U_{L3}~A_{M}A_{F}~U_{L}G_{F}~A_{M}U_{F}~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{L3}~A_{M}A_{F}~U_{L}G_{F}~A_{M}U_{F3}~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{L3}~A_{M}A_{F}~U_{L3}G_{F}~A_{M}U_{F3}~C_{M4}~A_{F}~G_{L}~G_{F}~U_{M3}~A_{F}~G_{M}
P-U_{M}~U_{F12}~A_{L}U_{F12}~C_{M}U_{F12}~A_{M}U_{L12}~A_{M}A_{F}~U_{L}G_{F}~A_{M}U_{F}~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{L12}~A_{M}A_{F}~U_{L}G_{F}~A_{M}U_{F12}~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{L12}~A_{M}A_{F}~U_{L12}G_{F}~A_{M}U_{F12}~C_{M4}~A_{F}~G_{L}~G_{F}~U_{M12}~A_{F}~G_{M}
P-U_{M}~U_{F18}~A_{L}U_{F18}~C_{M}U_{F18}~A_{M}U_{L18}~A_{M}A_{F}~U_{L}G_{F}~A_{M}U_{F}~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{L18}~A_{M}A_{F}~U_{L}G_{F}~A_{M}U_{F18}~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{L18}~A_{M}A_{F}~U_{L18}G_{F}~A_{M}U_{F18}~C_{M4}~A_{F}~G_{L}~G_{F}~U_{M18}~A_{F}~G_{M}
P-U_{M}~U_{F}~A_{L}U_{F3}~C_{M}U_{F3}~A_{M}U_{L3}~A_{M}A_{F}~U_{L}G_{F}~A_{M}U~C_{M4}~A_{F}~G_{L}~G_{F}~U_{M3}~A_{F}~G_{M}
P-U_{M}~U_{F3}~A_{L}U_{F3}~C_{M}U_{F}~A_{M}U_{L3}~A_{M}A_{F}~U_{L}G_{F}~A_{M}U~C_{M4}~A_{F}~G_{L}~G_{F}~U_{M3}~A_{F}~G_{M}
P-U_{M}~U_{F12}~A_{L}U_{F12}~C_{M}U_{F12}~A_{M}U_{L}~A_{M}A_{F}~U_{L}G_{F}~A_{M}U~C_{M4}~A_{F}~G_{L}~G_{F}~U_{M12}~A_{F}~G_{M}
P-U_{M}~U_{F12}~A_{L}U_{F12}~C_{M}U_{F12}~A_{M}U_{L12}~A_{M}A_{F}~U_{L}G_{F}~A_{M}U~C_{M}~A_{F}~G_{L}~G_{F}~U_{M12}~A_{F}~G_{M}
P-U_{M}~U_{F18}~A_{L}U_{F18}~C_{M4}U_{F18}~A_{M}U_{L18}~A_{M}A_{F}~U_{L}G_{F}~A_{M}U~C_{M4}~A_{F}~G_{L}~G_{F}~U_{M}~A_{F}~G_{M}
P-U_{M}~U_{F}~A_{L}U_{F18}~C_{M4}U_{F18}~A_{M}U_{L18}~A_{M}A_{F}~U_{L}G_{F}~A_{M}U~C_{M4}~A_{F}~G_{L}~G_{F}~U_{M}~A_{F}~G_{M}
P-U_{M}~U_{F3}~A_{L}U_{F3}~C_{M}U_{F3}~A_{M}U_{L3}~A_{M}A_{F}~U_{L}G_{F}~A_{M}U~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{L3}~A_{M}A_{F}~U_{L}G_{F}~A_{M}U~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{L3}~A_{M}A_{F}~U_{L3}G_{F}~A_{M}U~C_{M4}~A_{F}~G_{L}~G_{F}~U_{M3}~A_{F}~G_{M}
P-U_{M}~U_{F12}~A_{L}U_{F12}~C_{M}U_{F12}~A_{M}U_{L12}~A_{M}A_{F}~U_{L}G_{F}~A_{M}U~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{L12}~A_{M}A_{F}~U_{L}G_{F}~A_{M}U~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{L12}~A_{M}A_{F}~U_{L12}G_{F}~A_{M}U~C_{M4}~A_{F}~G_{L}~G_{F}~U_{M12}~A_{F}~G_{M}
P-U_{M}~U_{F18}~A_{L}U_{F18}~C_{M}U_{F18}~A_{M}U_{L18}~A_{M}A_{F}~U_{L}G_{F}~A_{M}U~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{L18}~A_{M}A_{F}~U_{L}G_{F}~A_{M}U~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{L18}~A_{M}A_{F}~U_{L18}G_{F}~A_{M}U~C_{M4}~A_{F}~G_{L}~G_{F}~U_{M18}~A_{F}~G_{M}
P-U_{M}~U_{M}~A_{L}U_{M3}~C_{F}U_{M3}~A_{F}U_{L3}~A_{F}A_{F}~U_{L}G_{F}~A_{M}U_{F}~C_{M4}~A_{F}~G_{L}~G_{F}~U_{M3}~A_{F}~G_{M}
P-U_{M}~U_{M3}~A_{L}U_{M3}~C_{F}U_{M}~A_{F}U_{L3}~A_{F}A_{F}~U_{L}G_{F}~A_{M}U_{F}~C_{M4}~A_{F}~G_{L}~G_{F}~U_{M3}~A_{F}~G_{M}
P-U_{M}~U_{M12}~A_{L}U_{M12}~C_{F}U_{M12}~A_{F}U_{L}~A_{F}A_{F}~U_{L}G_{F}~A_{M}U_{F}~C_{M4}~A_{F}~G_{L}~G_{F}~U_{M12}~A_{F}~G_{M}
P-U_{M}~U_{M12}~A_{L}U_{M12}~C_{F}U_{M12}~A_{F}U_{L12}~A_{F}A_{F}~U_{L}G_{F}~A_{M}U_{F}~C_{M}~A_{F}~G_{L}~G_{F}~U_{M12}~A_{F}~G_{M}
P-U_{M}~U_{M18}~A_{L}U_{M18}~C_{F4}U_{M18}~A_{F}U_{L18}~A_{F}A_{F}~U_{L}G_{F}~A_{M}U_{F}~C_{M4}~A_{F}~G_{L}~G_{F}~U_{M}~A_{F}~G_{M}
P-U_{M}~U_{M}~A_{L}U_{M18}~C_{F4}U_{M18}~A_{F}U_{L18}~A_{F}A_{F}~U_{L}G_{F}~A_{M}U_{F}~C_{M4}~A_{F}~G_{L}~G_{F}~U_{M}~A_{F}~G_{M}
P-U_{M}~U_{M}~A_{L}U_{M3}~C_{F}U_{M3}~A_{F}U_{L3}~A_{F}A_{F}~U_{L}G_{F}~A_{M}U_{F}~C_{M4}~A_{F}~G_{L}~G_{F}~U_{M3}~A_{F}~G_{M}
P-U_{M}~U_{M}~A_{L}U_{M3}~C_{F4}U_{M3}~A_{F}U_{L3}~A_{F}A_{F}~U_{L}G_{F}~A_{M}U_{F3}~C_{M4}~A_{F}~G_{L}~G_{F}~U_{M3}~A_{F}~G_{M}
P-U_{M}~U_{M}~A_{L}U_{M3}~C_{F4}U_{M3}~A_{F}U_{L3}~A_{F}A_{F}~U_{L3}G_{F}~A_{M}U_{F3}~C_{M4}~A_{F}~G_{L}~G_{F}~U_{M3}~A_{F}~G_{M}
P-U_{M}~U_{M}~A_{L}U_{M12}~C_{F}U_{M12}~A_{F}U_{L12}~A_{F}A_{F}~U_{L}G_{F}~A_{M}U_{F}~C_{M4}~A_{F}~G_{L}~G_{F}~U_{M12}~A_{F}~G_{M}
P-U_{M}~U_{M}~A_{L}U_{M12}~C_{F4}U_{M12}~A_{F}U_{L12}~A_{F}A_{F}~U_{L}G_{F}~A_{M}U_{F12}~C_{M4}~A_{F}~G_{L}~G_{F}~U_{M12}~A_{F}~G_{M}
P-U_{M}~U_{M}~A_{L}U_{M12}~C_{F4}U_{M12}~A_{F}U_{L12}~A_{F}A_{F}~U_{L12}G_{F}~A_{M}U_{F12}~C_{M4}~A_{F}~G_{L}~G_{F}~U_{M12}~A_{F}~G_{M}
P-U_{M}~U_{M}~A_{L}U_{M18}~C_{F}U_{M18}~A_{F}U_{L18}~A_{F}A_{F}~U_{L}G_{F}~A_{M}U_{F}~C_{M4}~A_{F}~G_{L}~G_{F}~U_{M18}~A_{F}~G_{M}
P-U_{M}~U_{M}~A_{L}U_{M18}~C_{F4}U_{M18}~A_{F}U_{L18}~A_{F}A_{F}~U_{L}G_{F}~A_{M}U_{F18}~C_{M4}~A_{F}~G_{L}~G_{F}~U_{M18}~A_{F}~G_{M}
P-U_{M}~U_{M}~A_{L}U_{M18}~C_{F4}U_{M18}~A_{F}U_{L18}~A_{F}A_{F}~U_{L18}G_{F}~A_{M}U_{F18}~C_{M4}~A_{F}~G_{L}~G_{F}~U_{M18}~A_{F}~G_{M}
P-U_{M}~U_{F}~A_{L}U_{F}~C_{M}U_{F}~A_{M}U_{F}~A_{L}A_{F}~U_{M}G_{F}~A_{L}U_{F}~C_{M}~A_{L}~G_{M}~G_{F}~U_{M}~A_{F}~G_{M}
P-U_{M}~U_{F}~A_{L}U_{F}~C_{M}U_{F}~A_{M}U_{F}~A_{L}A_{F}~U_{M}G_{F}~A_{L}U~C_{M}~A_{L}~G_{M}~G_{F}~U_{M}~A_{F}~G_{M}
P-U_{M}~U_{M}~A_{L}U_{M}~C_{F}U_{M}~A_{F}U_{M}~A_{L}A_{F}~U_{M}G_{F}~A_{L}U_{F}~C_{M}~A_{L}~G_{M}~G_{F}~U_{M}~A_{F}~G_{M}
P-U_{M}~U_{F}~A_{L}U_{F3}~C_{M}U_{F3}~A_{M}U_{F3}~A_{L}A_{F}~U_{M}G_{F}~A_{L}U_{F}~C_{M4}~A_{L}~G_{M}~G_{F}~U_{M3}~A_{F}~G_{M}
P-U_{M}~U_{F3}~A_{L}U_{F3}~C_{M}U_{F}~A_{M}U_{F3}~A_{L}A_{F}~U_{M}G_{F}~A_{L}U_{F}~C_{M4}~A_{L}~G_{M}~G_{F}~U_{M3}~A_{F}~G_{M}
P-U_{M}~U_{F12}~A_{L}U_{F12}~C_{M}U_{F12}~A_{M}U_{F}~A_{L}A_{F}~U_{M}G_{F}~A_{L}U_{F}~C_{M4}~A_{L}~G_{M}~G_{F}~U_{M12}~A_{F}~G_{M}
P-U_{M}~U_{F12}~A_{L}U_{F12}~C_{M}U_{F12}~A_{M}U_{F12}~A_{L}A_{F}~U_{M}G_{F}~A_{L}U_{F}~C_{M}~A_{L}~G_{M}~G_{F}~U_{M12}~A_{F}~G_{M}
P-U_{M}~U_{F18}~A_{L}U_{F18}~C_{M4}U_{F18}~A_{M}U_{F18}~A_{L}A_{F}~U_{M}G_{F}~A_{L}U_{F}~C_{M4}~A_{L}~G_{M}~G_{F}~U_{M}~A_{F}~G_{M}
P-U_{M}~U_{F}~A_{L}U_{F18}~C_{M4}U_{F18}~A_{M}U_{F18}~A_{L}A_{F}~U_{M}G_{F}~A_{L}U_{F}~C_{M4}~A_{L}~G_{M}~G_{F}~U_{M}~A_{F}~G_{M}
P-U_{M}~U_{F3}~A_{L}U_{F3}~C_{M}U_{F3}~A_{M}U_{F3}~A_{L}A_{F}~U_{M}G_{F}~A_{L}U_{F}~C_{M4}~A_{L}~G_{M}~G_{F}~U_{M3}~A_{F}~G_{M}
P-U_{M}~U_{F3}~A_{L}U_{F3}~C_{M4}U_{F3}~A_{M}U_{F3}~A_{L}A_{F}~U_{M}G_{F}~A_{L}U_{F3}~C_{M4}~A_{L}~G_{M}~G_{F}~U_{M3}~A_{F}~G_{M}
P-U_{M}~U_{F3}~A_{L}U_{F3}~C_{M4}U_{F3}~A_{M}U_{F3}~A_{L}A_{F}~U_{M3}G_{F}~A_{L}U_{F3}~C_{M4}~A_{L}~G_{M}~G_{F}~U_{M3}~A_{F}~G_{M}
P-U_{M}~U_{F12}~A_{L}U_{F12}~C_{M}U_{F12}~A_{M}U_{F12}~A_{L}A_{F}~U_{M}G_{F}~A_{L}U_{F}~C_{M4}~A_{L}~G_{M}~G_{F}~U_{M12}~A_{F}~G_{M}
P-U_{M}~U_{F12}~A_{L}U_{F12}~C_{M4}U_{F12}~A_{M}U_{F12}~A_{L}A_{F}~U_{M}G_{F}~A_{L}U_{F12}~C_{M4}~A_{L}~G_{M}~G_{F}~U_{M12}~A_{F}~G_{M}
P-U_{M}~U_{F12}~A_{L}U_{F12}~C_{M4}U_{F12}~A_{M}U_{F12}~A_{L}A_{F}~U_{M12}G_{F}~A_{L}U_{F12}~C_{M4}~A_{L}~G_{M}~G_{F}~U_{M12}~A_{F}~G_{M}
P-U_{M}~U_{F18}~A_{L}U_{F18}~C_{M}U_{F18}~A_{M}U_{F18}~A_{L}A_{F}~U_{M}G_{F}~A_{L}U_{F}~C_{M4}~A_{L}~G_{M}~G_{F}~U_{M18}~A_{F}~G_{M}
P-U_{M}~U_{F18}~A_{L}U_{F18}~C_{M4}U_{F18}~A_{M}U_{F18}~A_{L}A_{F}~U_{M}G_{F}~A_{L}U_{F18}~C_{M4}~A_{L}~G_{M}~G_{F}~U_{M18}~A_{F}~G_{M}
P-U_{M}~U_{F18}~A_{L}U_{F18}~C_{M}U_{F18}~A_{M}U_{F18}~A_{L}A_{F}~U_{M18}G_{F}~A_{L}U_{F18}~C_{M4}~A_{L}~G_{M}~G_{F}~U_{M18}~A_{F}~G_{M}
P-U_{M}~U_{F}~A_{L}U_{F3}~C_{M}U_{F3}~A_{M}U_{F3}~A_{L}A_{F}~U_{M}G_{F}~A_{L}U~C_{M4}~A_{L}~G_{M}~G_{F}~U_{M3}~A_{F}~G_{M}
P-U_{M}~U_{F3}~A_{L}U_{F3}~C_{M}U_{F}~A_{M}U_{F3}~A_{L}A_{F}~U_{M}G_{F}~A_{L}U~C_{M4}~A_{L}~G_{M}~G_{F}~U_{M3}~A_{F}~G_{M}
P-U_{M}~U_{F12}~A_{L}U_{F12}~C_{M}U_{F12}~A_{M}U_{F}~A_{L}A_{F}~U_{M}G_{F}~A_{L}U~C_{M4}~A_{L}~G_{M}~G_{F}~U_{M12}~A_{F}~G_{M}
P-U_{M}~U_{F12}~A_{L}U_{F12}~C_{M}U_{F12}~A_{M}U_{F12}~A_{L}A_{F}~U_{M}G_{F}~A_{L}U~C_{M}~A_{L}~G_{M}~G_{F}~U_{M12}~A_{F}~G_{M}
P-U_{M}~U_{F18}~A_{L}U_{F18}~C_{M4}U_{F18}~A_{M}U_{F18}~A_{L}A_{F}~U_{M}G_{F}~A_{L}U~C_{M4}~A_{L}~G_{M}~G_{F}~U_{M}~A_{F}~G_{M}
P-U_{M}~U_{F}~A_{L}U_{F18}~C_{M4}U_{F18}~A_{M}U_{F18}~A_{L}A_{F}~U_{M}G_{F}~A_{L}U~C_{M4}~A_{L}~G_{M}~G_{F}~U_{M}~A_{F}~G_{M}
P-U_{M}~U_{F3}~A_{L}U_{F3}~C_{M}U_{F3}~A_{M}U_{F3}~A_{L}A_{F}~U_{M}G_{F}~A_{L}U~C_{M4}~A_{L}~G_{M}~G_{F}~U_{M3}~A_{F}~G_{M}
P-U_{M}~U_{F3}~A_{L}U_{F3}~C_{M4}U_{F3}~A_{M}U_{F3}~A_{L}A_{F}~U_{M}G_{F}~A_{L}U~C_{M4}~A_{L}~G_{M}~G_{F}~U_{M3}~A_{F}~G_{M}
P-U_{M}~U_{F3}~A_{L}U_{F3}~C_{M4}U_{F3}~A_{M}U_{F3}~A_{L}A_{F}~U_{M3}G_{F}~A_{L}U~C_{M4}~A_{L}~G_{M}~G_{F}~U_{M3}~A_{F}~G_{M}
P-U_{M}~U_{F12}~A_{L}U_{F12}~C_{M}U_{F12}~A_{M}U_{F12}~A_{L}A_{F}~U_{M}G_{F}~A_{L}U~C_{M4}~A_{L}~G_{M}~G_{F}~U_{M12}~A_{F}~G_{M}
P-U_{M}~U_{F12}~A_{L}U_{F12}~C_{M4}U_{F12}~A_{M}U_{F12}~A_{L}A_{F}~U_{M}G_{F}~A_{L}U~C_{M4}~A_{L}~G_{M}~G_{F}~U_{M12}~A_{F}~G_{M}
P-U_{M}~U_{F12}~A_{L}U_{F12}~C_{M4}U_{F12}~A_{M}U_{F12}~A_{L}A_{F}~U_{M12}G_{F}~A_{L}U~C_{M4}~A_{L}~G_{M}~G_{F}~U_{M12}~A_{F}~G_{M}
P-U_{M}~U_{F18}~A_{L}U_{F18}~C_{M}U_{F18}~A_{M}U_{F18}~A_{L}A_{F}~U_{M}G_{F}~A_{L}U~C_{M4}~A_{L}~G_{M}~G_{F}~U_{M18}~A_{F}~G_{M}
P-U_{M}~U_{F18}~A_{L}U_{F18}~C_{M4}U_{F18}~A_{M}U_{F18}~A_{L}A_{F}~U_{M}G_{F}~A_{L}U~C_{M4}~A_{L}~G_{M}~G_{F}~U_{M18}~A_{F}~G_{M}
P-U_{M}~U_{F18}~A_{L}U_{F18}~C_{M4}U_{F18}~A_{M}U_{F18}~A_{L}A_{F}~U_{M18}G_{F}~A_{L}U~C_{M4}~A_{L}~G_{M}~G_{F}~U_{M18}~A_{F}~G_{M}
P-U_{M}~U_{M}~A_{L}U_{M3}~C_{F}U_{M3}~A_{F}U_{M3}~A_{L}A_{F}~U_{M}G_{F}~A_{L}U_{F}~C_{M4}~A_{L}~G_{M}~G_{F}~U_{M3}~A_{F}~G_{M}
P-U_{M}~U_{M3}~A_{L}U_{M3}~C_{F}U_{M}~A_{F}U_{M3}~A_{L}A_{F}~U_{M}G_{F}~A_{L}U_{F}~C_{M4}~A_{L}~G_{M}~G_{F}~U_{M3}~A_{F}~G_{M}
P-U_{M}~U_{M12}~A_{L}U_{M12}~C_{F}U_{M12}~A_{F}U_{M}~A_{L}A_{F}~U_{M}G_{F}~A_{L}U_{F}~C_{M4}~A_{L}~G_{M}~G_{F}~U_{M12}~A_{F}~G_{M}
P-U_{M}~U_{M12}~A_{L}U_{M12}~C_{F}U_{M12}~A_{F}U_{M12}~A_{L}A_{F}~U_{M}G_{F}~A_{L}U_{F}~C_{M}~A_{L}~G_{M}~G_{F}~U_{M12}~A_{F}~G_{M}
P-U_{M}~U_{M18}~A_{L}U_{M18}~C_{F4}U_{M18}~A_{F}U_{M18}~A_{L}A_{F}~U_{M}G_{F}~A_{L}U_{F}~C_{M4}~A_{L}~G_{M}~G_{F}~U_{M}~A_{F}~G_{M}
P-U_{M}~U_{M}~A_{L}U_{M18}~C_{F}U_{M18}~A_{F}U_{M18}~A_{L}A_{F}~U_{M}G_{F}~A_{L}U_{F}~C_{M4}~A_{L}~G_{M}~G_{F}~U_{M}~A_{F}~G_{M}
P-U_{M}~U_{M}~A_{L}U_{M3}~C_{F}U_{M3}~A_{F}U_{M3}~A_{L}A_{F}~U_{M}G_{F}~A_{L}U_{F}~C_{M4}~A_{L}~G_{M}~G_{F}~U_{M3}~A_{F}~G_{M}
P-U_{M}~U_{M}~A_{L}U_{M3}~C_{F4}U_{M3}~A_{F}U_{M3}~A_{L}A_{F}~U_{M}G_{F}~A_{L}U_{F3}~C_{M4}~A_{L}~G_{M}~G_{F}~U_{M3}~A_{F}~G_{M}
P-U_{M}~U_{M}~A_{L}U_{M3}~C_{F4}U_{M3}~A_{F}U_{M3}~A_{L}A_{F}~U_{M3}G_{F}~A_{L}U_{F3}~C_{M4}~A_{L}~G_{M}~G_{F}~U_{M3}~A_{F}~G_{M}
P-U_{M}~U_{M}~A_{L}U_{M12}~C_{F}U_{M12}~A_{F}U_{M12}~A_{L}A_{F}~U_{M}G_{F}~A_{L}U_{F}~C_{M4}~A_{L}~G_{M}~G_{F}~U_{M12}~A_{F}~G_{M}
P-U_{M}~U_{M}~A_{L}U_{M12}~C_{F4}U_{M12}~A_{F}U_{M12}~A_{L}A_{F}~U_{M}G_{F}~A_{L}U_{F12}~C_{M4}~A_{L}~G_{M}~G_{F}~U_{M12}~A_{F}~G_{M}
P-U_{M}~U_{M}~A_{L}U_{M12}~C_{F4}U_{M12}~A_{F}U_{M12}~A_{L}A_{F}~U_{M12}G_{F}~A_{L}U_{F12}~C_{M4}~A_{L}~G_{M}~G_{F}~U_{M12}~A_{F}~G_{M}
P-U_{M}~U_{M}~A_{L}U_{M18}~C_{F}U_{M18}~A_{F}U_{M18}~A_{L}A_{F}~U_{M}G_{F}~A_{L}U_{F}~C_{M4}~A_{L}~G_{M}~G_{F}~U_{M18}~A_{F}~G_{M}
P-U_{M}~U_{M}~A_{L}U_{M18}~C_{F4}U_{M15}~A_{F}U_{M18}~A_{L}A_{F}~U_{M}G_{F}~A_{L}U_{F18}~C_{M4}~A_{L}~G_{M}~G_{F}~U_{M18}~A_{F}~G_{M}
P-U_{M}~U_{M}~A_{L}U_{M18}~C_{F4}U_{M18}~A_{F}U_{M18}~A_{L}A_{F}~U_{M18}G_{F}~A_{L}U_{F18}~C_{M4}~A_{L}~G_{M}~G_{F}~U_{M18}~A_{F}~G_{M}
P-U_{M}~U_{F}~A_{M}U_{L}~C_{M}U_{F}~A_{M}U_{L}~A_{M}A_{F}~U_{L}G_{F}~A_{M}U_{F}~C_{L}~A_{F}~G_{M}~G_{L}~U_{M}~A_{F}~G_{M}
P-U_{M}~U_{F}~A_{M}U_{L}~C_{M}U_{F}~A_{M}U_{L}~A_{M}A_{F}~U_{L}G_{F}~A_{M}U_{F}~C_{L}~A_{F}~G_{M}~G_{L}~U_{M}~A_{F}~G_{M}
P-U_{M}~U_{M}~A_{F}U_{L}~C_{F}U_{M}~A_{F}U_{L}~A_{F}A_{F}~U_{L}G_{F}~A_{M}U_{F}~C_{L}~A_{F}~G_{M}~G_{L}~U_{M}~A_{F}~G_{M}
P-U_{M}~U_{F}~A_{M}U_{L3}~C_{M}U_{F3}~A_{M}U_{L3}~A_{M}A_{F}~U_{L}G_{F}~A_{M}U_{F}~C_{L4}~A_{F}~G_{M}~G_{L}~U_{M3}~A_{F}~G_{M}
P-U_{M}~U_{F3}~A_{M}U_{L3}~C_{M}U_{F}~A_{M}U_{L3}~A_{M}A_{F}~U_{L}G_{F}~A_{M}U_{F}~C_{L4}~A_{F}~G_{M}~G_{L}~U_{M3}~A_{F}~G_{M}
P-U_{M}~U_{F12}~A_{M}U_{L12}~C_{M}U_{F12}~A_{M}U_{L}~A_{M}A_{F}~U_{L}G_{F}~A_{M}U_{F}~C_{L4}~A_{F}~G_{M}~G_{L}~U_{M12}~A_{F}~G_{M}
P-U_{M}~U_{F12}~A_{M}U_{L12}~C_{M}U_{F12}~A_{M}U_{L12}~A_{M}A_{F}~U_{L}G_{F}~A_{M}U_{F}~C_{L}~A_{F}~G_{M}~G_{L}~U_{M12}~A_{F}~G_{M}
P-U_{M}~U_{F18}~A_{M}U_{L18}~C_{M4}U_{F18}~A_{M}U_{L18}~A_{M}A_{F}~U_{L}G_{F}~A_{M}U_{F}~C_{L4}~A_{F}~G_{M}~G_{L}~U_{M}~A_{F}~G_{M}
P-U_{M}~U_{F}~A_{M}U_{L18}~C_{M4}U_{F18}~A_{M}U_{L18}~A_{M}A_{F}~U_{L}G_{F}~A_{M}U_{F}~C_{L4}~A_{F}~G_{M}~G_{L}~U_{M}~A_{F}~G_{M}
P-U_{M}~U_{F3}~A_{M}U_{L3}~C_{M}U_{F3}~A_{M}U_{L3}~A_{M}A_{F}~U_{L}G_{F}~A_{M}U_{F}~C_{L4}~A_{F}~G_{M}~G_{L}~U_{M3}~A_{F}~G_{M}
P-U_{M}~U_{F3}~A_{M}U_{L3}~C_{M4}U_{F3}~A_{M}U_{L3}~A_{M}A_{F}~U_{L}G_{F}~A_{M}U_{F3}~C_{L4}~A_{F}~G_{M}~G_{L}~U_{M3}~A_{F}~G_{M}
P-U_{M}~U_{F3}~A_{M}U_{L3}~C_{M4}U_{F3}~A_{M}U_{L3}~A_{M}A_{F}~U_{L3}G_{F}~A_{M}U_{F3}~C_{L4}~A_{F}~G_{M}~G_{L}~U_{M3}~A_{F}~G_{M}
P-U_{M}~U_{F12}~A_{M}U_{L12}~C_{M}U_{F12}~A_{M}U_{L12}~A_{M}A_{F}~U_{L}G_{F}~A_{M}U_{F}~C_{L4}~A_{F}~G_{M}~G_{L}~U_{M12}~A_{F}~G_{M}
P-U_{M}~U_{F12}~A_{M}U_{L12}~C_{M4}U_{F12}~A_{M}U_{L12}~A_{M}A_{F}~U_{L}G_{F}~A_{M}U_{F12}~C_{L4}~A_{F}~G_{M}~G_{L}~U_{M12}~A_{F}~G_{M}
P-U_{M}~U_{F12}~A_{M}U_{L12}~C_{M4}U_{F12}~A_{M}U_{L12}~A_{M}A_{F}~U_{L12}G_{F}~A_{M}U_{F12}~C_{L4}~A_{F}~G_{M}~G_{L}~U_{M12}~A_{F}~G_{M}
P-U_{M}~U_{F18}~A_{M}U_{L18}~C_{M}U_{F18}~A_{M}U_{L18}~A_{M}A_{F}~U_{L}G_{F}~A_{M}U_{F}~C_{L4}~A_{F}~G_{M}~G_{L}~U_{M18}~A_{F}~G_{M}
P-U_{M}~U_{F18}~A_{M}U_{L18}~C_{M4}U_{F18}~A_{M}U_{L18}~A_{M}A_{F}~U_{L}G_{F}~A_{M}U_{F18}~C_{L4}~A_{F}~G_{M}~G_{L}~U_{M18}~A_{F}~G_{M}
P-U_{M}~U_{F18}~A_{M}U_{L18}~C_{M4}U_{F18}~A_{M}U_{L18}~A_{M}A_{F}~U_{L18}G_{F}~A_{M}U_{F18}~C_{L4}~A_{F}~G_{M}~G_{L}~U_{M18}~A_{F}~G_{M}
P-U_{M}~U_{F}~A_{M}U_{L3}~C_{M}U_{F3}~A_{M}U_{L3}~A_{M}A_{F}~U_{L}G_{F}~A_{M}U~C_{L4}~A_{F}~G_{M}~G_{L}~U_{M3}~A_{F}~G_{M}
P-U_{M}~U_{F3}~A_{M}U_{L3}~C_{M}U_{F}~A_{M}U_{L3}~A_{M}A_{F}~U_{L}G_{F}~A_{M}U~C_{L4}~A_{F}~G_{M}~G_{L}~U_{M3}~A_{F}~G_{M}
P-U_{M}~U_{F12}~A_{M}U_{L12}~C_{M}U_{F12}~A_{M}U_{L}~A_{M}A_{F}~U_{L}G_{F}~A_{M}U~C_{L4}~A_{F}~G_{M}~G_{L}~U_{M12}~A_{F}~G_{M}
P-U_{M}~U_{F12}~A_{M}U_{L12}~C_{M}U_{F12}~A_{M}U_{L12}~A_{M}A_{F}~U_{L}G_{F}~A_{M}U~C_{L}~A_{F}~G_{M}~G_{L}~U_{M12}~A_{F}~G_{M}
P-U_{M}~U_{F18}~A_{M}U_{L18}~C_{M4}U_{F18}~A_{M}U_{L18}~A_{M}A_{F}~U_{L}G_{F}~A_{M}U~C_{L4}~A_{F}~G_{M}~G_{L}~U_{M}~A_{F}~G_{M}
P-U_{M}~U_{F}~A_{M}U_{L18}~C_{M4}U_{F18}~A_{M}U_{L18}~A_{M}A_{F}~U_{L}G_{F}~A_{M}U~C_{L4}~A_{F}~G_{M}~G_{L}~U_{M}~A_{F}~G_{M}
P-U_{M}~U_{F3}~A_{M}U_{L3}~C_{M}U_{F3}~A_{M}U_{L3}~A_{M}A_{F}~U_{L}G_{F}~A_{M}U~C_{L4}~A_{F}~G_{M}~G_{L}~U_{M3}~A_{F}~G_{M}
P-U_{M}~U_{F3}~A_{M}U_{L3}~C_{M4}U_{F3}~A_{M}U_{L3}~A_{M}A_{F}~U_{L}G_{F}~A_{M}U~C_{L4}~A_{F}~G_{M}~G_{L}~U_{M3}~A_{F}~G_{M}
P-U_{M}~U_{F3}~A_{M}U_{L3}~C_{M4}U_{F3}~A_{M}U_{L3}~A_{M}A_{F}~U_{L3}G_{F}~A_{M}U~C_{L4}~A_{F}~G_{M}~G_{L}~U_{M3}~A_{F}~G_{M}
P-U_{M}~U_{F12}~A_{M}U_{L12}~C_{M}U_{F12}~A_{M}U_{L12}~A_{M}A_{F}~U_{L}G_{F}~A_{M}U~C_{L4}~A_{F}~G_{M}~G_{L}~U_{M12}~A_{F}~G_{M}
P-U_{M}~U_{F12}~A_{M}U_{L12}~C_{M4}U_{F12}~A_{M}U_{L12}~A_{M}A_{F}~U_{L}G_{F}~A_{M}U~C_{L4}~A_{F}~G_{M}~G_{L}~U_{M12}~A_{F}~G_{M}
P-U_{M}~U_{F12}~A_{M}U_{L12}~C_{M4}U_{F12}~A_{M}U_{L12}~A_{M}A_{F}~U_{L12}G_{F}~A_{M}U~C_{L4}~A_{F}~G_{M}~G_{L}~U_{M12}~A_{F}~G_{M}
P-U_{M}~U_{F18}~A_{M}U_{L18}~C_{M}U_{F18}~A_{M}U_{L18}~A_{M}A_{F}~U_{L}G_{F}~A_{M}U~C_{L4}~A_{F}~G_{M}~G_{L}~U_{M18}~A_{F}~G_{M}
P-U_{M}~U_{F18}~A_{M}U_{L18}~C_{M4}U_{F18}~A_{M}U_{L18}~A_{M}A_{F}~U_{L}G_{F}~A_{M}U~C_{L4}~A_{F}~G_{M}~G_{L}~U_{M18}~A_{F}~G_{M}
P-U_{M}~U_{F18}~A_{M}U_{L18}~C_{M4}U_{F18}~A_{M}U_{L18}~A_{M}A_{F}~U_{L18}G_{F}~A_{M}U~C_{L4}~A_{F}~G_{M}~G_{L}~U_{M18}~A_{F}~G_{M}
P-U_{M}~U_{M}~A_{F}U_{L3}~C_{F}U_{M3}~A_{F}U_{L3}~A_{F}A_{F}~U_{L}G_{F}~A_{M}U_{F}~C_{L4}~A_{F}~G_{M}~G_{L}~U_{M3}~A_{F}~G_{M}
P-U_{M}~U_{M3}~A_{F}U_{L3}~C_{F}U_{M}~A_{F}U_{L3}~A_{F}A_{F}~U_{L}G_{F}~A_{M}U_{F}~C_{L4}~A_{F}~G_{M}~G_{L}~U_{M3}~A_{F}~G_{M}
P-U_{M}~U_{M12}~A_{F}U_{L12}~C_{F}U_{M12}~A_{F}U_{L}~A_{F}A_{F}~U_{L}G_{F}~A_{M}U_{F}~C_{L4}~A_{F}~G_{M}~G_{L}~U_{M12}~A_{F}~G_{M}
P-U_{M}~U_{M12}~A_{F}U_{L12}~C_{F}U_{M12}~A_{F}U_{L12}~A_{F}A_{F}~U_{L}G_{F}~A_{M}U_{F}~C_{L}~A_{F}~G_{M}~G_{L}~U_{M12}~A_{F}~G_{M}
P-U_{M}~U_{M18}~A_{F}U_{L18}~C_{F4}U_{M18}~A_{F}U_{L18}~A_{F}A_{F}~U_{L}G_{F}~A_{M}U_{F}~C_{L4}~A_{F}~G_{M}~G_{L}~U_{M}~A_{F}~G_{M}
P-U_{M}~U_{M}~A_{F}U_{L18}~C_{F4}U_{M18}~A_{F}U_{L18}~A_{F}A_{F}~U_{L}G_{F}~A_{M}U_{F}~C_{L4}~A_{F}~G_{M}~G_{L}~U_{M}~A_{F}~G_{M}
P-U_{M}~U_{M}~A_{F}U_{L3}~C_{F}U_{M3}~A_{F}U_{L3}~A_{F}A_{F}~U_{L}G_{F}~A_{M}U_{F}~C_{L4}~A_{F}~G_{M}~G_{L}~U_{M3}~A_{F}~G_{M}
P-U_{M}~U_{M}~A_{F}U_{L3}~C_{F4}U_{M3}~A_{F}U_{L3}~A_{F}A_{F}~U_{L}G_{F}~A_{M}U_{F3}~C_{L4}~A_{F}~G_{M}~G_{L}~U_{M3}~A_{F}~G_{M}
P-U_{M}~U_{M}~A_{F}U_{L3}~C_{F4}U_{M3}~A_{F}U_{L3}~A_{F}A_{F}~U_{L3}G_{F}~A_{M}U_{F3}~C_{L4}~A_{F}~G_{M}~G_{L}~U_{M3}~A_{F}~G_{M}
P-U_{M}~U_{M}~A_{F}U_{L12}~C_{F}U_{M12}~A_{F}U_{L12}~A_{F}A_{F}~U_{L}G_{F}~A_{M}U_{F}~C_{L4}~A_{F}~G_{M}~G_{L}~U_{M12}~A_{F}~G_{M}
P-U_{M}~U_{M}~A_{F}U_{L12}~C_{F4}U_{M12}~A_{F}U_{L12}~A_{F}A_{F}~U_{L}G_{F}~A_{M}U_{F12}~C_{L4}~A_{F}~G_{M}~G_{L}~U_{M12}~A_{F}~G_{M}
P-U_{M}~U_{M}~A_{F}U_{L12}~C_{F4}U_{M12}~A_{F}U_{L12}~A_{F}A_{F}~U_{L12}G_{F}~A_{M}U_{F12}~C_{L4}~A_{F}~G_{M}~G_{L}~U_{M12}~A_{F}~G_{M}
P-U_{M}~U_{M}~A_{F}U_{L18}~C_{F}U_{M18}~A_{F}U_{L18}~A_{F}A_{F}~U_{L}G_{F}~A_{M}U_{F}~C_{L4}~A_{F}~G_{M}~G_{L}~U_{M18}~A_{F}~G_{M}
P-U_{M}~U_{M}~A_{F}U_{L18}~C_{F4}U_{M18}~A_{F}U_{L18}~A_{F}A_{F}~U_{L}G_{F}~A_{M}U_{F18}~C_{L4}~A_{F}~G_{M}~G_{L}~U_{M18}~A_{F}~G_{M}
P-U_{M}~U_{M}~A_{F}U_{L18}~C_{F4}U_{M18}~A_{F}U_{L18}~A_{F}A_{F}~U_{L18}G_{F}~A_{M}U_{F18}~C_{L4}~A_{F}~G_{M}~G_{L}~U_{M18}~A_{F}~G_{M}
P-U_{M}~U_{F}~A_{M}U_{F}~C_{M}U_{F}~A_{M}U_{F}~A_{M}A_{F}~U_{M}G_{F}~A_{M}U_{F}~C_{M}~A_{F}~G_{L}~G_{F}~U_{M}~A_{F}~G_{M}
P-U_{M}~U_{F}~A_{M}U_{F}~C_{M}U_{F}~A_{M}U_{F}~A_{M}A_{F}~U_{M}G_{F}~A_{M}U~C_{M}~A_{F}~G_{L}~G_{F}~U_{M}~A_{F}~G_{M}
P-U_{M}~U_{M}~A_{F}U_{M}~C_{F}U_{M}~A_{F}U_{M}~A_{F}A_{F}~U_{M}G_{F}~A_{M}U_{F}~C_{M}~A_{F}~G_{L}~G_{F}~U_{M}~A_{F}~G_{M}
P-U_{M}~U_{F3}~A_{M}U_{F3}~C_{M}U_{F3}~A_{M}U_{F3}~A_{M}A_{F}~U_{M}G_{F}~A_{M}U_{F}~C_{M4}~A_{F}~G_{L}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M}G_{F}~A_{M}U_{F3}~C_{M4}~A_{F}~G_{L}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M3}G_{F}~A_{M}U_{F3}~C_{M4}~A_{F}~G_{L}~G_{F}~U_{M3}~A_{F}~G_{M}
P-U_{M}~U_{F12}~A_{M}U_{F12}~C_{M}U_{F12}~A_{M}U_{F12}~A_{M}A_{F}~U_{M}G_{F}~A_{M}U_{F}~C_{M4}~A_{F}~G_{L}~G_{F}~U_{M12}~A_{F}~G_{M}
P-U_{M}~U_{F12}~A_{M}U_{F12}~C_{M4}U_{F12}~A_{M}U_{F12}~A_{M}A_{F}~U_{M}G_{F}~A_{M}U_{F12}~C_{M4}~A_{F}~G_{L}~G_{F}~U_{M12}~A_{F}~G_{M}
P-U_{M}~U_{F12}~A_{M}U_{F12}~C_{M4}U_{F12}~A_{M}U_{F12}~A_{M}A_{F}~U_{M12}G_{F}~A_{M}U_{F12}~C_{M4}~A_{F}~G_{L}~G_{F}~U_{M12}~A_{F}~G_{M}
P-U_{M}~U_{F18}~A_{M}U_{F18}~C_{M}U_{F18}~A_{M}U_{F18}~A_{M}A_{F}~U_{M}G_{F}~A_{M}U_{F}~C_{M4}~A_{F}~G_{L}~G_{F}~U_{M18}~A_{F}~G_{M}
P-U_{M}~U_{F18}~A_{M}U_{F18}~C_{M4}U_{F18}~A_{M}U_{F18}~A_{M}A_{F}~U_{M}G_{F}~A_{M}U_{F18}~C_{M4}~A_{F}~G_{L}~G_{F}~U_{M18}~A_{F}~G_{M}
P-U_{M}~U_{F18}~A_{M}U_{F18}~C_{M4}U_{F18}~A_{M}U_{F18}~A_{M}A_{F}~U_{M18}G_{F}~A_{M}U_{F18}~C_{M4}~A_{F}~G_{L}~G_{F}~U_{M18}~A_{F}~G_{M}
P-U_{M}~U_{F3}~A_{M}U_{F3}~C_{M}U_{F3}~A_{M}U_{F3}~A_{M}A_{F}~U_{M}G_{F}~A_{M}U~C_{M4}~A_{F}~G_{L}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M}G_{F}~A_{M}U~C_{M4}~A_{F}~G_{L}~G_{F}~U_{M3}~A_{F}~G_{M}
P-U_{M}~U_{F3}~A_{M}U_{F3}~C_{M4}U_{F3}~A_{M}U_{F3}~A_{M}A_{F}~U_{M3}G_{F}~A_{M}U~C_{M4}~A_{F}~G_{L}~G_{F}~U_{M3}~A_{F}~G_{M}
P-U_{M}~U_{F12}~A_{M}U_{F12}~C_{M}U_{F12}~A_{M}U_{F12}~A_{M}A_{F}~U_{M}G_{F}~A_{M}U~C_{M4}~A_{F}~G_{L}~G_{F}~U_{M12}~A_{F}~G_{M}
P-U_{M}~U_{F12}~A_{M}U_{F12}~C_{M4}U_{F12}~A_{M}U_{F12}~A_{M}A_{F}~U_{M}G_{F}~A_{M}U~C_{M4}~A_{F}~G_{L}~G_{F}~U_{M12}~A_{F}~G_{M}
P-U_{M}~U_{F12}~A_{M}U_{F12}~C_{M4}U_{F12}~A_{M}U_{F12}~A_{M}A_{F}~U_{M12}G_{F}~A_{M}U~C_{M4}~A_{F}~G_{L}~G_{F}~U_{M12}~A_{F}~G_{M}
P-U_{M}~U_{F18}~A_{M}U_{F18}~C_{M}U_{F18}~A_{M}U_{F18}~A_{M}A_{F}~U_{M}G_{F}~A_{M}U~C_{M4}~A_{F}~G_{L}~G_{F}~U_{M18}~A_{F}~G_{M}
P-U_{M}~U_{F18}~A_{M}U_{F18}~C_{M4}U_{F18}~A_{M}U_{F18}~A_{M}A_{F}~U_{M}G_{F}~A_{M}U~C_{M4}~A_{F}~G_{L}~G_{F}~U_{M18}~A_{F}~G_{M}
P-U_{M}~U_{F18}~A_{M}U_{F18}~C_{M4}U_{F18}~A_{M}U_{F18}~A_{M}A_{F}~U_{M18}G_{F}~A_{M}U~C_{M4}~A_{F}~G_{L}~G_{F}~U_{M18}~A_{F}~G_{M}
P-U_{M}~U_{M}~A_{F}U_{M3}~C_{F}U_{M3}~A_{F}U_{M3}~A_{F}A_{F}~U_{M}G_{F}~A_{M}U_{F}~C_{M4}~A_{F}~G_{L}~G_{F}~U_{M3}~A_{F}~G_{M}
P-U_{M}~U_{M}~A_{F}U_{M3}~C_{F4}U_{M3}~A_{F}U_{M3}~A_{F}A_{F}~U_{M}G_{F}~A_{M}U_{F3}~C_{M4}~A_{F}~G_{L}~G_{F}~U_{M3}~A_{F}~G_{M}
P-U_{M}~U_{M}~A_{F}U_{M3}~C_{F4}U_{M3}~A_{F}U_{M3}~A_{F}A_{F}~U_{M3}G_{F}~A_{M}U_{F3}~C_{M4}~A_{F}~G_{L}~G_{F}~U_{M3}~A_{F}~G_{M}
P-U_{M}~U_{M}~A_{F}U_{M12}~C_{F}U_{M12}~A_{F}U_{M12}~A_{F}A_{F}~U_{M}G_{F}~A_{M}U_{F}~C_{M4}~A_{F}~G_{L}~G_{F}~U_{M12}~A_{F}~G_{M}
P-U_{M}~U_{M}~A_{F}U_{M12}~C_{F4}U_{M12}~A_{F}U_{M12}~A_{F}A_{F}~U_{M}G_{F}~A_{M}U_{F12}~C_{M4}~A_{F}~G_{L}~G_{F}~U_{M12}~A_{F}~G_{M}
P-U_{M}~U_{M}~A_{F}U_{M12}~C_{F4}U_{M12}~A_{F}U_{M12}~A_{F}A_{F}~U_{M12}G_{F}~A_{M}U_{F12}~C_{M4}~A_{F}~G_{L}~G_{F}~U_{M12}~A_{F}~G_{M}
P-U_{M}~U_{M}~A_{F}U_{M18}~C_{F}U_{M18}~A_{F}U_{M18}~A_{F}A_{F}~U_{M}G_{F}~A_{M}U_{F}~C_{M4}~A_{F}~G_{L}~G_{F}~U_{M18}~A_{F}~G_{M}
P-U_{M}~U_{M}~A_{F}U_{M18}~C_{F4}U_{M18}~A_{F}U_{M18}~A_{F}A_{F}~U_{M}G_{F}~A_{M}U_{F18}~C_{M4}~A_{F}~G_{L}~G_{F}~U_{M18}~A_{F}~G_{M}
P-U_{M}~U_{M}~A_{F}U_{M18}~C_{F4}U_{M18}~A_{F}U_{M18}~A_{F}A_{F}~U_{M18}G_{F}~A_{M}U_{F18}~C_{M4}~A_{F}~G_{L}~G_{F}~U_{M18}~A_{F}~G_{M}
P-U_{M}~U_{F}~A_{M}U_{F}~C_{M}U_{F}~A_{M}U_{F}~A_{M}A_{F}~U_{M}G_{F}~A_{M}U_{F}~C_{M}~A_{L}~G_{M}~G_{L}~U_{M}~A_{F}~G_{M}
P-U_{M}~U_{F}~A_{M}U_{F}~C_{M}U_{F}~A_{M}U_{F}~A_{M}A_{F}~U_{M}G_{F}~A_{M}U~C_{M}~A_{L}~G_{M}~G_{L}~U_{M}~A_{F}~G_{M}
P-U_{M}~U_{M}~A_{F}U_{M}~C_{F}U_{M}~A_{F}U_{M}~A_{F}A_{F}~U_{M}G_{F}~A_{M}U_{F}~C_{M}~A_{L}~G_{M}~G_{L}~U_{M}~A_{F}~G_{M}
P-U_{M}~U_{F3}~A_{M}U_{F3}~C_{M}U_{F3}~A_{M}U_{F3}~A_{M}A_{F}~U_{M}G_{F}~A_{M}U_{F}~C_{M4}~A_{L}~G_{M}~G_{L}~U_{M3}~A_{F}~G_{M}
P-U_{M}~U_{F3}~A_{M}U_{F3}~C_{M4}U_{F3}~A_{M}U_{F3}~A_{M}A_{F}~U_{M}G_{F}~A_{M}U_{F3}~C_{M4}~A_{L}~G_{M}~G_{L}~U_{M3}~A_{F}~G_{M}
P-U_{M}~U_{F3}~A_{M}U_{F3}~C_{M4}U_{F3}~A_{M}U_{F3}~A_{M}A_{F}~U_{M3}G_{F}~A_{M}U_{F3}~C_{M4}~A_{L}~G_{M}~G_{L}~U_{M3}~A_{F}~G_{M}
P-U_{M}~U_{F12}~A_{M}U_{F12}~C_{M}U_{F12}~A_{M}U_{F12}~A_{M}A_{F}~U_{M}G_{F}~A_{M}U_{F}~C_{M4}~A_{L}~G_{M}~G_{L}~U_{M12}~A_{F}~G_{M}
P-U_{M}~U_{F12}~A_{M}U_{F12}~C_{M4}U_{F12}~A_{M}U_{F12}~A_{M}A_{F}~U_{M}G_{F}~A_{M}U_{F12}~C_{M4}~A_{L}~G_{M}~G_{L}~U_{M12}~A_{F}~G_{M}
P-U_{M}~U_{F12}~A_{M}U_{F12}~C_{M4}U_{F12}~A_{M}U_{F12}~A_{M}A_{F}~U_{M12}G_{F}~A_{M}U_{F12}~C_{M4}~A_{L}~G_{M}~G_{L}~U_{M12}~A_{F}~G_{M}
P-U_{M}~U_{F18}~A_{M}U_{F18}~C_{M}U_{F18}~A_{M}U_{F18}~A_{M}A_{F}~U_{M}G_{F}~A_{M}U_{F}~C_{M4}~A_{L}~G_{M}~G_{L}~U_{M18}~A_{F}~G_{M}
P-U_{M}~U_{F18}~A_{M}U_{F18}~C_{M4}U_{F18}~A_{M}U_{F18}~A_{M}A_{F}~U_{M}G_{F}~A_{M}U_{F18}~C_{M4}~A_{L}~G_{M}~G_{L}~U_{M18}~A_{F}~G_{M}
P-U_{M}~U_{F18}~A_{M}U_{F18}~C_{M4}U_{F18}~A_{M}U_{F18}~A_{M}A_{F}~U_{M18}G_{F}~A_{M}U_{F18}~C_{M4}~A_{L}~G_{M}~G_{L}~U_{M18}~A_{F}~G_{M}
P-U_{M}~U_{F3}~A_{M}U_{F3}~C_{M}U_{F3}~A_{M}U_{F3}~A_{M}A_{F}~U_{M}G_{F}~A_{M}U~C_{M4}~A_{L}~G_{M}~G_{L}~U_{M3}~A_{F}~G_{M}
P-U_{M}~U_{F3}~A_{M}U_{F3}~C_{M4}U_{F3}~A_{M}U_{F3}~A_{M}A_{F}~U_{M}G_{F}~A_{M}U~C_{M4}~A_{L}~G_{M}~G_{L}~U_{M3}~A_{F}~G_{M}
P-U_{M}~U_{F3}~A_{M}U_{F3}~C_{M4}U_{F3}~A_{M}U_{F3}~A_{M}A_{F}~U_{M3}G_{F}~A_{M}U~C_{M4}~A_{L}~G_{M}~G_{L}~U_{M3}~A_{F}~G_{M}
P-U_{M}~U_{F12}~A_{M}U_{F12}~C_{M}U_{F12}~A_{M}U_{F12}~A_{M}A_{F}~U_{M}G_{F}~A_{M}U~C_{M4}~A_{L}~G_{M}~G_{L}~U_{M12}~A_{F}~G_{M}
P-U_{M}~U_{F12}~A_{M}U_{F12}~C_{M4}U_{F12}~A_{M}U_{F12}~A_{M}A_{F}~U_{M}G_{F}~A_{M}U~C_{M4}~A_{L}~G_{M}~G_{L}~U_{M12}~A_{F}~G_{M}
P-U_{M}~U_{F12}~A_{M}U_{F12}~C_{M4}U_{F12}~A_{M}U_{F12}~A_{M}A_{F}~U_{M12}G_{F}~A_{M}U~C_{M4}~A_{L}~G_{M}~G_{L}~U_{M12}~A_{F}~G_{M}
P-U_{M}~U_{F18}~A_{M}U_{F18}~C_{M}U_{F18}~A_{M}U_{F18}~A_{M}A_{F}~U_{M}G_{F}~A_{M}U~C_{M4}~A_{L}~G_{M}~G_{L}~U_{M18}~A_{F}~G_{M}
P-U_{M}~U_{F18}~A_{M}U_{F18}~C_{M4}U_{F18}~A_{M}U_{F18}~A_{M}A_{F}~U_{M}G_{F}~A_{M}U~C_{M4}~A_{L}~G_{M}~G_{L}~U_{M18}~A_{F}~G_{M}
P-U_{M}~U_{F18}~A_{M}U_{F18}~C_{M4}U_{F18}~A_{M}U_{F18}~A_{M}A_{F}~U_{M18}G_{F}~A_{M}U~C_{M4}~A_{L}~G_{M}~G_{L}~U_{M18}~A_{F}~G_{M}
P-U_{M}~U_{M}~A_{F}U_{M3}~C_{F}U_{M3}~A_{F}U_{M3}~A_{F}A_{F}~U_{M}G_{F}~A_{M}U_{F}~C_{M4}~A_{L}~G_{M}~G_{L}~U_{M3}~A_{F}~G_{M}
P-U_{M}~U_{M}~A_{F}U_{M3}~C_{F4}U_{M3}~A_{F}U_{M3}~A_{F}A_{F}~U_{M}G_{F}~A_{M}U_{F3}~C_{M4}~A_{L}~G_{M}~G_{L}~U_{M3}~A_{F}~G_{M}
P-U_{M}~U_{M}~A_{F}U_{M3}~C_{F4}U_{M3}~A_{F}U_{M3}~A_{F}A_{F}~U_{M3}G_{F}~A_{M}U_{F3}~C_{M4}~A_{L}~G_{M}~G_{L}~U_{M3}~A_{F}~G_{M}
P-U_{M}~U_{M}~A_{F}U_{M12}~C_{F}U_{M12}~A_{F}U_{M12}~A_{F}A_{F}~U_{M}G_{F}~A_{M}U_{F}~C_{M4}~A_{L}~G_{M}~G_{L}~U_{M12}~A_{F}~G_{M}
P-U_{M}~U_{M}~A_{F}U_{M12}~C_{F4}U_{M12}~A_{F}U_{M12}~A_{F}A_{F}~U_{M}G_{F}~A_{M}U_{F12}~C_{M4}~A_{L}~G_{M}~G_{L}~U_{M12}~A_{F}~G_{M}
P-U_{M}~U_{M}~A_{F}U_{M12}~C_{F4}U_{M12}~A_{F}U_{M12}~A_{F}A_{F}~U_{M12}G_{F}~A_{M}U_{F12}~C_{M4}~A_{L}~G_{M}~G_{L}~U_{M12}~A_{F}~G_{M}
P-U_{M}~U_{M}~A_{F}U_{M18}~C_{F}U_{M18}~A_{F}U_{M18}~A_{F}A_{F}~U_{M}G_{F}~A_{M}U_{F}~C_{M4}~A_{L}~G_{M}~G_{L}~U_{M18}~A_{F}~G_{M}
P-U_{M}~U_{M}~A_{F}U_{M18}~C_{F4}U_{M18}~A_{F}U_{M18}~A_{F}A_{F}~U_{M}G_{F}~A_{M}U_{F18}~C_{M4}~A_{L}~G_{M}~G_{L}~U_{M18}~A_{F}~G_{M}
P-U_{M}~U_{M}~A_{F}U_{M18}~C_{F4}U_{M18}~A_{F}U_{M18}~A_{F}A_{F}~U_{M18}G_{F}~A_{M}U_{F18}~C_{M4}~A_{L}~G_{M}~G_{L}~U_{M18}~A_{F}~G_{M}
P-U_{M}~U_{F}~A_{M}U_{F}~C_{M}U_{F}~A_{L}U_{F}~A_{L}A_{F}~U_{M}G_{L}~A_{M}U_{F}~C_{M}~A_{F}~G_{L}~G_{F}~U_{M}~A_{F}~G_{M}
P-U_{M}~U_{F}~A_{M}U_{F}~C_{M}U_{F}~A_{L}U_{F}~A_{L}A_{F}~U_{M}G_{L}~A_{M}U~C_{M}~A_{F}~G_{L}~G_{F}~U_{M}~A_{F}~G_{M}
P-U_{M}~U_{M}~A_{F}U_{M}~C_{F}U_{M}~A_{L}U_{M}~A_{L}A_{F}~U_{M}G_{L}~A_{M}U_{F}~C_{M}~A_{F}~G_{L}~G_{F}~U_{M}~A_{F}~G_{M}
P-U_{M}~U_{F3}~A_{M}U_{F3}~C_{M}U_{F3}~A_{L}U_{F3}~A_{L}A_{F}~U_{M}G_{L}~A_{M}U_{F}~C_{M4}~A_{F}~G_{L}~G_{F}~U_{M3}~A_{F}~G_{M}
P-U_{M}~U_{F3}~A_{M}U_{F3}~C_{M4}U_{F3}~A_{L}U_{F3}~A_{L}A_{F}~U_{M}G_{L}~A_{M}U_{F3}~C_{M4}~A_{F}~G_{L}~G_{F}~U_{M3}~A_{F}~G_{M}
P-U_{M}~U_{F3}~A_{M}U_{F3}~C_{M4}U_{F3}~A_{L}U_{F3}~A_{L}A_{F}~U_{M3}G_{L}~A_{M}U_{F3}~C_{M4}~A_{F}~G_{L}~G_{F}~U_{M3}~A_{F}~G_{M}
P-U_{M}~U_{F12}~A_{M}U_{F12}~C_{M}U_{F12}~A_{L}U_{F12}~A_{L}A_{F}~U_{M}G_{L}~A_{M}U_{F}~C_{M4}~A_{F}~G_{L}~G_{F}~U_{M12}~A_{F}~G_{M}
P-U_{M}~U_{F12}~A_{M}U_{F12}~C_{M4}U_{F12}~A_{L}U_{F12}~A_{L}A_{F}~U_{M}G_{L}~A_{M}U_{F12}~C_{M4}~A_{F}~G_{L}~G_{F}~U_{M12}~A_{F}~G_{M}
P-U_{M}~U_{F12}~A_{M}U_{F12}~C_{M4}U_{F12}~A_{L}U_{F12}~A_{L}A_{F}~U_{M12}G_{L}~A_{M}U_{F12}~C_{M4}~A_{F}~G_{L}~G_{F}~U_{M12}~A_{F}~G_{M}
P-U_{M}~U_{F18}~A_{M}U_{F18}~C_{M}U_{F18}~A_{L}U_{F18}~A_{L}A_{F}~U_{M}G_{L}~A_{M}U_{F}~C_{M4}~A_{F}~G_{L}~G_{F}~U_{M18}~A_{F}~G_{M}
P-U_{M}~U_{F18}~A_{M}U_{F18}~C_{M4}U_{F18}~A_{L}U_{F18}~A_{L}A_{F}~U_{M}G_{L}~A_{M}U_{F18}~C_{M4}~A_{F}~G_{L}~G_{F}~U_{M18}~A_{F}~G_{M}
P-U_{M}~U_{F18}~A_{M}U_{F18}~C_{M4}U_{F18}~A_{L}U_{F18}~A_{L}A_{F}~U_{M18}G_{L}~A_{M}U_{F18}~C_{M4}~A_{F}~G_{L}~G_{F}~U_{M18}~A_{F}~G_{M}
P-U_{M}~U_{F3}~A_{M}U_{F3}~C_{M}U_{F3}~A_{L}U_{F3}~A_{L}A_{F}~U_{M}G_{L}~A_{M}U~C_{M4}~A_{F}~G_{L}~G_{F}~U_{M3}~A_{F}~G_{M}
P-U_{M}~U_{F3}~A_{M}U_{F3}~C_{M4}U_{F3}~A_{L}U_{F3}~A_{L}A_{F}~U_{M}G_{L}~A_{M}U~C_{M4}~A_{F}~G_{L}~G_{F}~U_{M3}~A_{F}~G_{M}
P-U_{M}~U_{F3}~A_{M}U_{F3}~C_{M4}U_{F3}~A_{L}U_{F3}~A_{L}A_{F}~U_{M3}G_{L}~A_{M}U~C_{M4}~A_{F}~G_{L}~G_{F}~U_{M3}~A_{F}~G_{M}
P-U_{M}~U_{F12}~A_{M}U_{F12}~C_{M}U_{F12}~A_{L}U_{F12}~A_{L}A_{F}~U_{M}G_{L}~A_{M}U~C_{M4}~A_{F}~G_{L}~G_{F}~U_{M12}~A_{F}~G_{M}
P-U_{M}~U_{F12}~A_{M}U_{F12}~C_{M4}U_{F12}~A_{L}U_{F12}~A_{L}A_{F}~U_{M}G_{L}~A_{M}U~C_{M4}~A_{F}~G_{L}~G_{F}~U_{M12}~A_{F}~G_{M}
P-U_{M}~U_{F12}~A_{M}U_{F12}~C_{M4}U_{F12}~A_{L}U_{F12}~A_{L}A_{F}~U_{M12}G_{L}~A_{M}U~C_{M4}~A_{F}~G_{L}~G_{F}~U_{M12}~A_{F}~G_{M}
P-U_{M}~U_{F18}~A_{M}U_{F18}~C_{M}U_{F18}~A_{L}U_{F18}~A_{L}A_{F}~U_{M}G_{L}~A_{M}U~C_{M4}~A_{F}~G_{L}~G_{F}~U_{M18}~A_{F}~G_{M}
P-U_{M}~U_{F18}~A_{M}U_{F18}~C_{M4}U_{F18}~A_{L}U_{F18}~A_{L}A_{F}~U_{M}G_{L}~A_{M}U~C_{M4}~A_{F}~G_{L}~G_{F}~U_{M18}~A_{F}~G_{M}
P-U_{M}~U_{F18}~A_{M}U_{F18}~C_{M4}U_{F18}~A_{L}U_{F18}~A_{L}A_{F}~U_{M18}G_{L}~A_{M}U~C_{M4}~A_{F}~G_{L}~G_{F}~U_{M18}~A_{F}~G_{M}
P-U_{M}~U_{M}~A_{F}U_{M3}~C_{F}U_{M3}~A_{L}U_{F3}~A_{L}A_{F}~U_{M}G_{L}~A_{M}U_{F}~C_{M4}~A_{F}~G_{L}~G_{F}~U_{M3}~A_{F}~G_{M}
P-U_{M}~U_{M}~A_{F}U_{M3}~C_{F4}U_{M3}~A_{L}U_{F3}~A_{L}A_{F}~U_{M}G_{L}~A_{M}U_{F3}~C_{M4}~A_{F}~G_{L}~G_{F}~U_{M3}~A_{F}~G_{M}
P-U_{M}~U_{M}~A_{F}U_{M3}~C_{F4}U_{M3}~A_{L}U_{F3}~A_{L}A_{F}~U_{M3}G_{L}~A_{M}U_{F3}~C_{M4}~A_{F}~G_{L}~G_{F}~U_{M3}~A_{F}~G_{M}
P-U_{M}~U_{M}~A_{F}U_{M12}~C_{F}U_{M12}~A_{L}U_{F12}~A_{L}A_{F}~U_{M}G_{L}~A_{M}U_{F}~C_{M4}~A_{F}~G_{L}~G_{F}~U_{M12}~A_{F}~G_{M}
P-U_{M}~U_{M}~A_{F}U_{M12}~C_{F4}U_{M12}~A_{L}U_{F12}~A_{L}A_{F}~U_{M}G_{L}~A_{M}U_{F12}~C_{M4}~A_{F}~G_{L}~G_{F}~U_{M12}~A_{F}~G_{M}
P-U_{M}~U_{M}~A_{F}U_{M12}~C_{F4}U_{M12}~A_{L}U_{F12}~A_{L}A_{F}~U_{M12}G_{L}~A_{M}U_{F12}~C_{M4}~A_{F}~G_{L}~G_{F}~U_{M12}~A_{F}~G_{M}
P-U_{M}~U_{M}~A_{F}U_{M18}~C_{F}U_{M18}~A_{L}U_{F18}~A_{L}A_{F}~U_{M}G_{L}~A_{M}U_{F}~C_{M4}~A_{F}~G_{L}~G_{F}~U_{M18}~A_{F}~G_{M}
P-U_{M}~U_{M}~A_{F}U_{M18}~C_{F4}U_{M18}~A_{L}U_{F18}~A_{L}A_{F}~U_{M}G_{L}~A_{M}U_{F18}~C_{M4}~A_{F}~G_{L}~G_{F}~U_{M18}~A_{F}~G_{M}
P-U_{M}~U_{M}~A_{F}U_{M18}~C_{F4}U_{M18}~A_{L}U_{F18}~A_{L}A_{F}~U_{M18}G_{L}~A_{M}U_{F18}~C_{M4}~A_{F}~G_{L}~G_{F}~U_{M18}~A_{F}~G_{M}
P-U_{M}~U_{F}~A_{L}U_{F}~C_{M}U_{F}~A_{M}U_{F}~A_{M}A_{F}~U_{M}G_{F}~A_{L}U_{F}~C_{M}~A_{F}~G_{L}~G_{F}~U_{M}~A_{F}~G_{M}
P-U_{M}~U_{F}~A_{L}U_{F}~C_{M}U_{F}~A_{M}U_{F}~A_{M}A_{F}~U_{M}G_{F}~A_{L}U~C_{M}~A_{F}~G_{L}~G_{F}~U_{M}~A_{F}~G_{M}
P-U_{M}~U_{M}~A_{L}U_{M}~C_{F}U_{M}~A_{F}U_{M}~A_{F}A_{F}~U_{M}G_{F}~A_{L}U_{F}~C_{M}~A_{F}~G_{L}~G_{F}~U_{M}~A_{F}~G_{M}
P-U_{M}~U_{F3}~A_{L}U_{F3}~C_{M}U_{F3}~A_{M}U_{F3}~A_{M}A_{F}~U_{M}G_{F}~A_{L}U_{F}~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{F3}~A_{M}A_{F}~U_{M}G_{F}~A_{L}U_{F3}~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{F3}~A_{M}A_{F}~U_{M3}G_{F}~A_{L}U_{F3}~C_{M4}~A_{F}~G_{L}~G_{F}~U_{M3}~A_{F}~G_{M}
P-U_{M}~U_{F12}~A_{L}U_{F12}~C_{M}U_{F12}~A_{M}U_{F12}~A_{M}A_{F}~U_{M}G_{F}~A_{L}U_{F}~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{F12}~A_{M}A_{F}~U_{M}G_{F}~A_{L}U_{F12}~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{F12}~A_{M}A_{F}~U_{M12}G_{F}~A_{L}U_{F12}~C_{M4}~A_{F}~G_{L}~G_{F}~U_{M12}~A_{F}~G_{M}
P-U_{M}~U_{F18}~A_{L}U_{F18}~C_{M}U_{F18}~A_{M}U_{F18}~A_{M}A_{F}~U_{M}G_{F}~A_{L}U_{F}~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{F18}~A_{M}A_{F}~U_{M}G_{F}~A_{L}U_{F18}~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{F18}~A_{M}A_{F}~U_{M18}G_{F}~A_{L}U_{F18}~C_{M4}~A_{F}~G_{L}~G_{F}~U_{M18}~A_{F}~G_{M}
P-U_{M}~U_{F3}~A_{L}U_{F3}~C_{M}U_{F3}~A_{M}U_{F3}~A_{M}A_{F}~U_{M}G_{F}~A_{L}U~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{F3}~A_{M}A_{F}~U_{M}G_{F}~A_{L}U~C_{M4}~A_{F}~G_{L}~G_{F}~U_{M3}~A_{F}~G_{M}
P-U_{M}~U_{F3}~A_{L}U_{F3}~C_{M4}U_{F3}~A_{M}U_{F3}~A_{M}A_{F}~U_{M3}G_{F}~A_{L}U~C_{M4}~A_{F}~G_{L}~G_{F}~U_{M3}~A_{F}~G_{M}
P-U_{M}~U_{F12}~A_{L}U_{F12}~C_{M}U_{F12}~A_{M}U_{F12}~A_{M}A_{F}~U_{M}G_{F}~A_{L}U~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{F12}~A_{M}A_{F}~U_{M}G_{F}~A_{L}U~C_{M4}~A_{F}~G_{L}~G_{F}~U_{M12}~A_{F}~G_{M}
P-U_{M}~U_{F12}~A_{L}U_{F12}~C_{M4}U_{F12}~A_{M}U_{F12}~A_{M}A_{F}~U_{M12}G_{F}~A_{L}U~C_{M4}~A_{F}~G_{L}~G_{F}~U_{M12}~A_{F}~G_{M}
P-U_{M}~U_{F18}~A_{L}U_{F18}~C_{M}U_{F18}~A_{M}U_{F18}~A_{M}A_{F}~U_{M}G_{F}~A_{L}U~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{F18}~A_{M}A_{F}~U_{M}G_{F}~A_{L}U ~C_{M4}~A_{F}~G_{L}~G_{F}~U_{M18}~A_{F}~G_{M}
P-U_{M}~U_{F18}~A_{L}U_{F18}~C_{M4}U_{F18}~A_{M}U_{F18}~A_{M}A_{F}~U_{M18}G_{F}~A_{L}U ~C_{M4}~A_{F}~G_{L}~G_{F}~U_{M18}~A_{F}~G_{M}
P-U_{M}~U_{M}~A_{L}U_{M3}~C_{F}U_{M3}~A_{F}U_{M3}~A_{F}A_{F}~U_{M}G_{F}~A_{L}U_{F}~C_{M4}~A_{F}~G_{L}~G_{F}~U_{M3}~A_{F}~G_{M}
P-U_{M}~U_{M}~A_{L}U_{M3}~C_{F4}U_{M3}~A_{F}U_{M3}~A_{F}A_{F}~U_{M}G_{F}~A_{L}U_{F3}~C_{M4}~A_{F}~G_{L}~G_{F}~U_{M3}~A_{F}~G_{M}
P-U_{M}~U_{M}~A_{L}U_{M3}~C_{F4}U_{M3}~A_{F}U_{M3}~A_{F}A_{F}~U_{M3}G_{F}~A_{L}U_{F3}~C_{M4}~A_{F}~G_{L}~G_{F}~U_{M3}~A_{F}~G_{M}
P-U_{M}~U_{M}~A_{L}U_{M12}~C_{F}U_{M12}~A_{F}U_{M12}~A_{F}A_{F}~U_{M}G_{F}~A_{L}U_{F}~C_{M4}~A_{F}~G_{L}~G_{F}~U_{M12}~A_{F}~G_{M}
P-U_{M}~U_{M}~A_{L}U_{M12}~C_{F4}U_{M12}~A_{F}U_{M12}~A_{F}A_{F}~U_{M}G_{F}~A_{L}U_{F12}~C_{M4}~A_{F}~G_{L}~G_{F}~U_{M12}~A_{F}~G_{M}
P-U_{M}~U_{M}~A_{L}U_{M12}~C_{F4}U_{M12}~A_{F}U_{M12}~A_{F}A_{F}~U_{M12}G_{F}~A_{L}U_{F12}~C_{M4}~A_{F}~G_{L}~G_{F}~U_{M12}~A_{F}~G_{M}
P-U_{M}~U_{M}~A_{L}U_{M18}~C_{F}U_{M18}~A_{F}U_{M18}~A_{F}A_{F}~U_{M}G_{F}~A_{L}U_{F}~C_{M4}~A_{F}~G_{L}~G_{F}~U_{M18}~A_{F}~G_{M}
P-U_{M}~U_{M}~A_{L}U_{M18}~C_{F4}U_{M18}~A_{F}U_{M18}~A_{F}A_{F}~U_{M}G_{F}~A_{L}U_{F18}~C_{M4}~A_{F}~G_{L}~G_{F}~U_{M18}~A_{F}~G_{M}
P-U_{M}~U_{M}~A_{L}UM₁₈~C_{F4}U_{M18}~A_{F}U_{M18}~A_{P}A_{F}~U_{M18}G_{F}~A_{L}U_{F18}~C_{M4}~A_{F}~G_{L}~G_{F}~U_{M18}~A_{F}~G_{M}
P-U_{M}~U_{F}~A_{L}U_{F}~C_{M}U_{F}~A_{L}U_{F}~A_{L}A_{F}~U_{M}G_{F}~A_{L}U_{F}~C_{M}~A_{L}~G_{M}~G_{L}~U_{M}~A_{F}~G_{M}
P-U_{M}~U_{F}~A_{L}U_{F}~C_{M}U_{F}~A_{L}U_{F}~A_{L}A_{F}~U_{M}G_{F}~A_{L}U ~C_{M}~A_{L}~G_{M}~G_{L}~U_{M}~A_{F}~G_{M}
P-U_{M}~U_{M}~A_{L}U_{M}~C_{F}U_{M}~A_{L}U_{M}~A_{L}A_{F}~U_{M}G_{F}~A_{L}U_{F}~C_{M}~A_{L}~G_{M}~G_{L}~U_{M}~A_{F}~G_{M}
P-U_{M}~U_{F3}~A_{L}U_{F3}~C_{M}U_{F3}~A_{L}U_{F3}~A_{L}A_{F}~U_{M}G_{F}~A_{L}U_{F}~C_{M4}~A_{L}~G_{F}~G_{L}~U_{M3}~A_{F}~G_{M}
P-U_{M}~U_{F3}~A_{L}U_{F3}~C_{M4}U_{F3}~A_{L}U_{F3}~A_{L}A_{F}~U_{M}G_{F}~A_{L}U_{F3}~C_{M4}~A_{L}~G_{F}~G_{L}~U_{M3}~A_{F}~G_{M}
P-U_{M}~U_{F3}~A_{L}U_{F3}~C_{M4}U_{F3}~A_{L}U_{F3}~A_{L}A_{F}~U_{M3}G_{F}~A_{L}U_{F3}~C_{M4}~A_{L}~G_{F}~G_{L}~U_{M3}~A_{F}~G_{M}
P-U_{M}~U_{F12}~A_{L}U_{F12}~C_{M}U_{F12}~A_{L}U_{F12}~A_{L}A_{F}~U_{M}G_{F}~A_{L}U_{F}~C_{M4}~A_{L}~G_{F}~G_{L}~U_{M12}~A_{F}~G_{M}
P-U_{M}~U_{F12}~A_{L}U_{F12}~C_{M4}U_{F12}~A_{L}U_{F12}~A_{L}A_{F}~U_{M}G_{F}~A_{L}U_{F12}~C_{M4}~A_{L}~G_{F}~G_{L}~U_{M12}~A_{F}~G_{M}
P-U_{M}~U_{F12}~A_{L}U_{F12}~C_{M4}U_{F12}~A_{L}U_{F12}~A_{L}A_{F}~U_{M12}G_{F}~A_{L}U_{F12}~C_{M4}~A_{L}~G_{F}~G_{L}~U_{M12}~A_{F}~G_{M}
P-U_{M}~U_{F18}~A_{L}U_{F18}~C_{M}U_{F18}~A_{L}U_{F18}~A_{L}A_{F}~U_{M}G_{F}~A_{L}U_{F}~C_{M4}~A_{L}~G_{F}~G_{L}~U_{M18}~A_{F}~G_{M}
P-U_{M}~U_{F18}~A_{L}U_{F18}~C_{M4}U_{F18}~A_{L}U_{F18}~A_{L}A_{F}~U_{M}G_{F}~A_{L}U_{F18}~C_{M4}~A_{L}~G_{F}~G_{L}~U_{M18}~A_{F}~G_{M}
P-U_{M}~U_{F18}~A_{L}U_{F18}~C_{M4}U_{F18}~A_{L}U_{F18}~A_{L}A_{F}~U_{M18}G_{F}~A_{L}U_{F18}~C_{M4}~A_{L}~G_{F}~G_{L}~U_{M18}~A_{F}~G_{M}
P-U_{M}~U_{F3}~A_{L}U_{F3}~C_{M}U_{F3}~A_{L}U_{F3}~A_{L}A_{F}~U_{M}G_{F}~A_{L}U ~C_{M4}~A_{L}~G_{F}~G_{L}~U_{M3}~A_{F}~G_{M}
P-U_{M}~U_{F3}~A_{L}U_{F3}~C_{M4}U_{F3}~A_{L}U_{F3}~A_{L}A_{F}~U_{M}G_{F}~A_{L}U ~C_{M4}~A_{L}~G_{F}~G_{L}~U_{M3}~A_{F}~G_{M}
P-U_{M}~U_{F3}~A_{L}U_{F3}~C_{M4}U_{F3}~A_{L}U_{F3}~A_{L}A_{F}~U_{M3}G_{F}~A_{L}U ~C_{M4}~A_{L}~G_{F}~G_{L}~U_{M3}~A_{F}~G_{M}
P-U_{M}~U_{F12}~A_{L}U_{F12}~C_{M}U_{F12}~A_{L}U_{F12}~A_{L}A_{F}~U_{M}G_{F}~A_{L}U ~C_{M4}~A_{L}~G_{F}~G_{L}~U_{M12}~A_{F}~G_{M}
P-U_{M}~U_{F12}~A_{L}U_{F12}~C_{M4}U_{F12}~A_{L}U_{F12}~A_{L}A_{F}~U_{M}G_{F}~A_{L}U ~C_{M4}~A_{L}~G_{F}~G_{L}~U_{M12}~A_{F}~G_{M}
P-U_{M}~U_{F12}~A_{L}U_{F12}~C_{M4}U_{F12}~A_{L}U_{F12}~A_{L}A_{F}~U_{M12}G_{F}~A_{L}U ~C_{M4}~A_{L}~G_{F}~G_{L}~U_{M12}~A_{F}~G_{M}
P-U_{M}~U_{F18}~A_{L}U_{F18}~C_{M}U_{F18}~A_{L}U_{F18}~A_{L}A_{F}~U_{M}G_{F}~A_{L}U ~C_{M4}~A_{L}~G_{F}~G_{L}~U_{M18}~A_{F}~G_{M}
P-U_{M}~U_{F18}~A_{L}U_{F18}~C_{M4}U_{F18}~A_{L}U_{F18}~A_{L}A_{F}~U_{M}G_{F}~A_{L}U ~C_{M4}~A_{L}~G_{F}~G_{L}~U_{M18}~A_{F}~G_{M}
P-U_{M}~U_{F18}~A_{L}U_{F18}~C_{M4}U_{F18}~A_{L}U_{F18}~A_{L}A_{F}~U_{M18}G_{F}~A_{L}U ~C_{M4}~A_{L}~G_{F}~G_{L}~U_{M18}~A_{F}~G_{M}
P-U_{M}~U_{M}~A_{L}U_{M3}~C_{F}U_{M3}~A_{L}U_{F3}~A_{L}A_{F}~U_{M}G_{F}~A_{L}U_{F}~C_{M4}~A_{L}~G_{F}~G_{L}~U_{M3}~A_{F}~G_{M}
P-U_{M}~U_{M}~A_{L}U_{M3}~C_{F4}U_{M3}~A_{L}U_{F3}~A_{L}A_{F}~U_{M}G_{F}~A_{L}U_{F3}~C_{M4}~A_{L}~G_{F}~G_{L}~U_{M3}~A_{F}~G_{M}
P-U_{M}~U_{M}~A_{L}U_{M3}~C_{F4}U_{M3}~A_{L}U_{F3}~A_{L}A_{F}~U_{M3}G_{F}~A_{L}U_{F3}~C_{M4}~A_{L}~G_{F}~G_{L}~U_{M3}~A_{F}~G_{M}
P-U_{M}~U_{M}~A_{L}U_{M12}~C_{F}U_{M12}~A_{L}U_{F12}~A_{L}A_{F}~U_{M}G_{F}~A_{L}U_{F}~C_{M4}~A_{L}~G_{F}~G_{L}~U_{M12}~A_{F}~G_{M}
P-U_{M}~U_{M}~A_{L}U_{M12}~C_{F4}U_{M12}~A_{L}U_{F12}~A_{L}A_{F}~U_{M}G_{F}~A_{L}U_{F12}~C_{M4}~A_{L}~G_{F}~G_{L}~U_{M12}~A_{F}~G_{M}
P-U_{M}~U_{M}~A_{L}U_{M12}~C_{F4}U_{M12}~A_{L}U_{F12}~A_{L}A_{F}~U_{M12}G_{F}~A_{L}U_{F12}~C_{M4}~A_{L}~G_{F}~G_{L}~U_{M12}~A_{F}~G_{M}
P-U_{M}~U_{M}~A_{L}U_{M18}~C_{F}U_{M18}~A_{L}U_{F18}~A_{L}A_{F}~U_{M}G_{F}~A_{L}U_{F}~C_{M4}~A_{L}~G_{F}~G_{L}~U_{M18}~A_{F}~G_{M}
P-U_{M}~U_{M}~A_{L}U_{M18}~C_{F4}U_{M18}~A_{L}U_{F18}~A_{L}A_{F}~U_{M}G_{F}~A_{L}U_{F18}~C_{M4}~A_{L}~G_{F}~G_{L}~U_{M18}~A_{F}~G_{M}
P-U_{M}~U_{M}~A_{L}U_{M18}~C_{F4}U_{M18}~A_{L}U_{F18}~A_{L}A_{F}~U_{M18}G_{F}~A_{L}U_{F18}~C_{M4}~A_{L}~G_{F}~G_{L}~U_{M18}~A_{F}~G_{M}
P-U_{M}~U_{F}~A_{M}U_{F}~C_{L}U_{F}~A_{M}U_{L}~A_{M}A_{F}~U_{M}G_{F}~A_{M}U_{F}~C_{L}~A_{F}~G_{M}~G_{L}~U_{M}~A_{F}~G_{M}
P-U_{M}~U_{F}~A_{M}U_{F}~C_{L}U_{F}~A_{M}U_{L}~A_{M}A_{F}~U_{M}G_{F}~A_{M}U ~C_{L}~A_{F}~G_{M}~G_{L}~U_{M}~A_{F}~G_{M}
P-U_{M}~U_{M}~A_{F}U_{M}~C_{L}U_{M}~A_{F}U_{L}~A_{F}A_{F}~U_{M}G_{F}~A_{M}U_{F}~C_{L}~A_{F}~G_{M}~G_{L}~U_{M}~A_{F}~G_{M}
P-U_{M}~U_{F3}~A_{M}U_{F3}~C_{L}U_{F3}~A_{M}U_{L}~A_{M}A_{F}~U_{M}G_{F}~A_{M}U_{F}~C_{L4}~A_{F}~G_{M}~G_{L}~U_{M3}~A_{F}~G_{M}
P-U_{M}~U_{F3}~A_{M}U_{F3}~C_{L}U_{F3}~A_{M}U_{L}~A_{M}A_{F}~U_{M}G_{F}~A_{M}U_{F3}~C_{L4}~A_{F}~G_{M}~G_{L}~U_{M3}~A_{F}~G_{M}
P-U_{M}~U_{F3}~A_{M}U_{F3}~C_{L}U_{F3}~A_{M}U_{L}~A_{M}A_{F}~U_{M3}G_{F}~A_{M}U_{F3}~C_{L4}~A_{F}~G_{M}~G_{L}~U_{M3}~A_{F}~G_{M}
P-U_{M}~U_{F12}~A_{M}U_{F12}~C_{L}U_{F12}~A_{M}U_{L}~A_{M}A_{F}~U_{M}G_{F}~A_{M}U_{F}~C_{L4}~A_{F}~G_{M}~G_{L}~U_{M12}~A_{F}~G_{M}
P-U_{M}~U_{F12}~A_{M}U_{F12}~C_{L}U_{F12}~A_{M}U_{L}~A_{M}A_{F}~U_{M}G_{F}~A_{M}U_{F12}~C_{L4}~A_{F}~G_{M}~G_{L}~U_{M12}~A_{F}~G_{M}
P-U_{M}~U_{F12}~A_{M}U_{F12}~C_{L}U_{F12}~A_{M}U_{L}~A_{M}A_{F}~U_{M12}G_{F}~A_{M}U_{F12}~C_{L4}~A_{F}~G_{M}~G_{L}~U_{M12}~A_{F}~G_{M}
P-U_{M}~U_{F18}~A_{M}U_{F18}~C_{L}U_{F18}~A_{M}U_{L}~A_{M}A_{F}~U_{M}G_{F}~A_{M}U_{F}~C_{L4}~A_{F}~G_{M}~G_{L}~U_{M18}~A_{F}~G_{M}
P-U_{M}~U_{F18}~A_{M}U_{F18}~C_{L}U_{F18}~A_{M}U_{L}~A_{M}A_{F}~U_{M}G_{F}~A_{M}U_{F18}~C_{L4}~A_{F}~G_{M}~G_{L}~U_{M18}~A_{F}~G_{M}
P-U_{M}~U_{F18}~A_{M}U_{F18}~C_{L}U_{F18}~A_{M}U_{L}~A_{M}A_{F}~U_{M}G_{F}~A_{M}U_{F18}~C_{L4}~A_{F}~G_{M}~G_{L}~U_{M18}~A_{F}~G_{M}
P-U_{M}~U_{F3}~A_{M}U_{F3}~C_{L}U_{F3}~A_{M}U_{L}~A_{M}A_{F}~U_{M}G_{F}~A_{M}U ~C_{L4}~A_{F}~G_{M}~G_{L}~U_{M3}~A_{F}~G_{M}
P-U_{M}~U_{F3}~A_{M}U_{F3}~C_{L}U_{F3}~A_{M}U_{L}~A_{M}A_{F}~U_{M}G_{F}~A_{M}U ~C_{L4}~A_{F}~G_{M}~G_{L}~U_{M3}~A_{F}~G_{M}
P-U_{M}~U_{F3}~A_{M}U_{F3}~C_{L}U_{F3}~A_{M}U_{L}~A_{M}A_{F}~U_{M3}G_{F}~A_{M}U ~C_{L4}~A_{F}~G_{M}~G_{L}~U_{M3}~A_{F}~G_{M}
P-U_{M}~U_{F3}~A_{M}U_{F3}~C_{L}U_{F3}~A_{M}U_{L}~A_{M}A_{F}~U_{M}G_{F}~A_{M}U ~C_{L}~A_{F}~G_{M}~G_{L}~U_{M3}~A_{F}~G_{M}
P-U_{M}~U_{F3}~A_{M}U_{F3}~C_{L}U_{F3}~A_{M}U_{L}~A_{M}A_{F}~U_{M3}G_{F}~A_{M}U ~C_{L}~A_{F}~G_{M}~G_{L}~U_{M3}~A_{F}~G_{M}
P-U_{M}~U_{F12}~A_{M}U_{F12}~C_{L}U_{F12}~A_{M}U_{L}~A_{M}A_{F}~U_{M}G_{F}~A_{M}U ~C_{L4}~A_{F}~G_{M}~G_{L}~U_{M12}~A_{F}~G_{M}
P-U_{M}~U_{F12}~A_{M}U_{F12}~C_{L}U_{F12}~A_{M}U_{L}~A_{M}A_{F}~U_{M}G_{F}~A_{M}U ~C_{L4}~A_{F}~G_{M}~G_{L}~U_{M12}~A_{F}~G_{M}
P-U_{M}~U_{F12}~A_{M}U_{F12}~C_{L}U_{F12}~A_{M}U_{L}~A_{M}A_{F}~U_{M12}G_{F}~A_{M}U ~C_{L4}~A_{F}~G_{M}~G_{L}~U_{M12}~A_{F}~G_{M}
P-U_{M}~U_{F12}~A_{M}U_{F12}~C_{L}U_{F12}~A_{M}U_{L}~A_{M}A_{F}~U_{M12}G_{F}~A_{M}U ~C_{L}~A_{F}~G_{M}~G_{L}~U_{M12}~A_{F}~G_{M}
P-U_{M}~U_{F18}~A_{M}U_{F18}~C_{L}U_{F18}~A_{M}U_{L}~A_{M}A_{F}~U_{M}G_{F}~A_{M}U ~C_{L4}~A_{F}~G_{M}~G_{L}~U_{M18}~A_{F}~G_{M}
P-U_{M}~U_{F18}~A_{M}U_{F18}~C_{L}U_{F18}~A_{M}U_{L}~A_{M}A_{F}~U_{M}G_{F}~A_{M}U ~C_{L4}~A_{F}~G_{M}~G_{L}~U_{M18}~A_{F}~G_{M}
P-U_{M}~U_{F18}~A_{M}U_{F18}~C_{L}U_{F18}~A_{M}U_{L}~A_{M}A_{F}~U_{M18}G_{F}~A_{M}U ~C_{L4}~A_{F}~G_{M}~G_{L}~U_{M18}~A_{F}~G_{M}
P-U_{M}~U_{M}~A_{F}U_{M3}~C_{L}U_{M3}~A_{F}U_{L}~A_{F}A_{F}~U_{M}G_{F}~A_{M}U_{F}~C_{L4}~A_{F}~G_{M}~G_{L}~U_{M3}~A_{F}~G_{M}
P-U_{M}~U_{M}~A_{F}U_{M3}~C_{L}U_{M3}~A_{F}U_{L}~A_{F}A_{F}~U_{M}G_{F}~A_{M}U_{F3}~C_{L4}~A_{F}~G_{M}~G_{L}~U_{M3}~A_{F}~G_{M}
P-U_{M}~U_{M}~A_{F}U_{M3}~C_{L}U_{M3}~A_{F}U_{L}~A_{F}A_{F}~U_{M3}G_{F}~A_{M}U_{F3}~C_{L4}~A_{F}~G_{M}~G_{L}~U_{M3}~A_{F}~G_{M}
P-U_{M}~U_{M}~A_{F}U_{M12}~C_{L}U_{M12}~A_{F}U_{L}~A_{F}A_{F}~U_{M}G_{F}~A_{M}U_{F}~C_{L4}~A_{F}~G_{M}~G_{L}~U_{M12}~A_{F}~G_{M}
P-U_{M}~U_{M}~A_{F}U_{M12}~C_{L}U_{M12}~A_{F}U_{L}~A_{F}A_{F}~U_{M}G_{F}~A_{M}U_{F12}~C_{L4}~A_{F}~G_{M}~G_{L}~U_{M12}~A_{F}~G_{M}
P-U_{M}~U_{M}~A_{F}U_{M12}~C_{L}U_{M12}~A_{F}U_{L}~A_{F}A_{F}~U_{M12}G_{F}~A_{M}U_{F12}~C_{L4}~A_{F}~G_{M}~G_{L}~U_{M12}~A_{F}~G_{M}
P-U_{M}~U_{M}~A_{F}U_{M18}~C_{L}U_{M18}~A_{F}U_{L}~A_{F}A_{F}~U_{M}G_{F}~A_{M}U_{F}~C_{L4}~A_{F}~G_{M}~G_{L}~U_{M18}~A_{F}~G_{M}
P-U_{M}~U_{M}~A_{F}U_{M18}~C_{L}U_{M18}~A_{F}U_{L}~A_{F}A_{F}~U_{M}G_{F}~A_{M}U_{F18}~C_{L4}~A_{F}~G_{M}~G_{L}~U_{M18}~A_{F}~G_{M}
P-U_{M}~U_{M}~A_{F}U_{M18}~C_{L}U_{M18}~A_{F}U_{L}~A_{F}A_{F}~U_{M18}G_{F}~A_{M}U_{F18}~C_{L4}~A_{F}~G_{M}~G_{L}~U_{M18}~A_{F}~G_{M}
P-U_{M}~U_{F}~A_{M}U_{F}~C_{L}U_{F}~A_{M}U_{L}~A_{M}A_{L}~U_{M}G_{F}~A_{M}U ~C_{L}~A_{F}~G_{M}~G_{L}~U_{M}~A_{F}~G_{M}
P-U_{M}~U_{F12}~A_{M}U_{F12}~C_{L}U_{F12}~A_{M}U_{L}~A_{M}_{A}_{L}~U_{M}G_{F}~A_{M}U ~C_{L}~A_{F}~G_{M}~G_{L}~U_{M12}~A_{F}~G_{M}
P-U_{M}~U_{F12}~A_{M}U_{F12}~C_{L}U_{F12}~A_{M}U_{L}~A_{M}A_{L}~U_{M12}G_{F}~A_{M}U ~C_{L}~A_{F}~G_{M}~G_{L}~U_{M12}~A_{F}~G_{M}
P-U_{M}~U_{F18}~A_{M}U_{F18}~C_{L}U_{F18}~A_{M}U_{L}~A_{M}A_{L}~U_{M}G_{F}~A_{M}U ~C_{L}~A_{F}~G_{M}~G_{L}~U_{M18}~A_{F}~G_{M}
P-U_{M}~U_{F18}~A_{M}U_{F18}~C_{L}U_{F18}~A_{M}U_{L}~A_{M}A_{L}~U_{M18}G_{F}~A_{M}U ~C_{L}~A_{F}~G_{M}~G_{L}~U_{M18}~A_{F}~G_{M}
P-U_{M}~U_{F3}~A_{M}U_{F3}~C_{L}U_{F3}~A_{M}U_{L}~A_{M}A_{L}~U_{M}G_{F}~A_{M}U ~C_{L}~A_{F}~G_{M}~G_{L}~U_{M3}~A_{F}~G_{M}
P-U_{M}~U_{F3}~A_{M}U_{F3}~C_{L}U_{F3}~A_{M}U_{L}~A_{M}A_{L}~U_{M3}G_{F}~A_{M}U ~C_{L}~A_{F}~G_{M}~G_{L}~U_{M3}~A_{F}~G_{M}

### Table 11

### Examples of Murine Factor VII ags-siRNA Compositions

### A. Murine Factor VII Antisense Sequence:

UUAAGACUUGAGAUGAUCC

Lima et al. (Cell 150: 883-94, 2012) used this sequence to generate ssRNAi compounds against human/murine PTEN including the following:

5′-VP-Tₘₒₑ~U_{F}~A_{M}A_{F}~G_{M}A_{F}~C_{M}U_{F}~U_{M}G_{F}~A_{M}G_{F}~A_{M}U_{F}~G_{M}~A_{F}~U_{M}~C_{F}~C_{M}~Aₘₒₑ~Aₘₒₑ

With the 5′-VP, T and 2′-MOE modifications removed and replaced by P, U and 2′-O-methyl:

P-U_{M}~U_{F}~A_{M}A_{F}~G_{M}A_{F}~C_{M}U_{F}~U_{M}G_{F}~A_{M}G_{F}~A_{M}U_{F}~G_{M}~A_{F}~U_{M}~C_{F}~C_{M}~A_{M}~A_{M}

Any of the 5′-end nucleosides (P-U_{M}) shown in the examples of agsRNAi compounds in Table 11 can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 11 with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the compounds in Table 11 can any of those units and linkages provided for herein for overhang precursors.

B. Examples of Application of AGSD Rules to this Compound:

Compositions of Matter Suitable for Administration to a Subject
with or without a Protective Carrier and for Any Other use
as Provided for Herein (5′-3′)

P-U_{M}~U_{F}~A_{M}A_{F}~G_{M}A_{F}~C_{M4}U_{F3}~U_{M}G_{F}~A_{M}G_{F}~A_{M}U_{F3}~G_{M}~A_{F}~U_{M3}~C_{F}~C_{M4}~A_{M}~A_{M}
P-U_{M}~U_{F}~A_{M}A_{F}~G_{M}A_{F}~C_{M4}U_{F3}~U_{M}G_{F}~A_{M}G_{F}~A_{M}U_{F3}~G_{M}~A_{F}~U_{M3}~C_{F4}~C_{M}~A_{M}~A_{M}
P-U_{M}~U_{F}~A_{M}A_{F}~G_{M}A_{F}~C_{M}U_{F3}~U_{M3}G_{F}~A_{M}G_{F}~A_{M}U_{F3}~G_{M}~A_{F}~U_{M}~C_{F4}~C_{M}~A_{M}~A_{M}
P-U_{M}~U_{F}~A_{M}A_{F}~G_{M}A_{F}~C_{M}U_{F3}~U_{M3}G_{F}~A_{M}G_{F}~A_{M}U_{F}~G_{M}~A_{F}~U_{M3}~C_{F}~C_{M4}~A_{M}~A_{M}
P-U_{M}~U_{F3}~A_{M}A_{F}~G_{M}A_{F}~C_{M}U_{F}~U_{M3}G_{F}~A_{M}G_{F}~A_{M}U_{F}~G_{M}~A_{F}~U_{M}~C_{F4}~C_{M}~A_{M}~A_{M}
P-U_{M}~U_{F3}~A_{M}A_{F}~G_{M}A_{F}~C_{M4}U_{F3}~U_{M}G_{F}~A_{M}G_{F}~A_{M}U_{F3}~G_{M}~A_{F}~U_{M3}~C_{F}~C_{M4}~A_{M}~A_{M}
P-U_{M}~U_{F3}~A_{M}A_{F}~G_{M}A_{F}~C_{M4}U_{F}~U_{M3}G_{F}~A_{M}G_{F}~A_{M}U_{F3}~G_{M}~A_{F}~U_{M}~C_{F4}~C_{M}~A_{M}~A_{M}
P-U_{M}~U_{F3}~A_{M}A_{F}~G_{M}A_{F}~C_{M4}U_{F3}~U_{M3}G_{F}~A_{M}G_{F}~A_{M}U_{F3}~G_{M}~A_{F}~U_{M}~C_{F4}~C_{M4}~A_{M}~A_{M}
P-U_{M}~U_{F}~A_{M}A_{F}~G_{M}A_{F}~C_{M4}U_{F3}~U_{M}G_{F}~A_{M}G_{F}~A_{M}U~G_{M}~A_{F}~U_{M3}~C_{F}~C_{M4}~A_{M}~A_{M}
P-U_{M}~U_{F}~A_{M}A_{F}~G_{M}A_{F}~C_{M4}U_{F3}~U_{M}G_{F}~A_{M}G_{F}~A_{M}U~G_{M}~A_{F}~U_{M3}~C_{F4}~C_{M}~A_{M}~A_{M}
P-U_{M}~U_{F}~A_{M}A_{F}~G_{M}A_{F}~C_{M}U_{F3}~U_{M3}G_{F}~A_{M}G_{F}~A_{M}U~G_{M}~A_{F}~U_{M}~C_{F4}~C_{M}~A_{M}~A_{M}
P-U_{M}~U_{F}~A_{M}A_{F}~G_{M}A_{F}~C_{M}U_{F3}~U_{M3}G_{F}~A_{M}G_{F}~A_{M}U~G_{M}~A_{F}~U_{M3}~C_{F}~C_{M4}~A_{M}~A_{M}
P-U_{M}~U_{F3}~A_{M}A_{F}~G_{M}A_{F}~C_{M}U_{F}~U_{M3}G_{F}~A_{M}G_{F}~A_{M}U~G_{M}~A_{F}~U_{M}~C_{F4}~C_{M}~A_{M}~A_{M}
P-U_{M}~U_{F3}~A_{M}A_{F}~G_{M}A_{F}~C_{M4}U_{F3}~U_{M}G_{F}~A_{M}G_{F}~A_{M}U~G_{M}~A_{F}~U_{M3}~C_{F}~C_{M4}~A_{M}~A_{M}
P-U_{M}~U_{F3}~A_{M}A_{F}~G_{M}A_{F}~C_{M4}U_{F}~U_{M3}G_{F}~A_{M}G_{F}~A_{M}U~G_{M}~A_{F}~U_{M}~C_{F4}~C_{M}~A_{M}~A_{M}
P-U_{M}~U_{F3}~A_{M}A_{F}~G_{M}A_{F}~C_{M4}U_{F3}~U_{M3}G_{F}~A_{M}G_{F}~A_{M}U~G_{M}~A_{F}~U_{M}~C_{F4}~C_{M4}~A_{M}~A_{M}
P-U_{M}~U_{F}~A_{M}A_{F}~G_{M}A_{F}~C_{M4}U_{F12}~U_{M}G_{F}~A_{M}G_{F}~A_{M}U_{F12}~G_{M}~A_{F}~U_{M12}~C_{F}~C_{M4}~A_{M}~A_{M}
P-U_{M}~U_{F}~A_{M}A_{F}~G_{M}A_{F}~C_{M4}U_{F12}~U_{M}G_{F}~A_{M}G_{F}~A_{M}U_{F12}~G_{M}~A_{F}~U_{M12}~C_{F4}~C_{M}~A_{M}~A_{M}
P-U_{M}~U_{F}~A_{M}A_{F}~G_{M}A_{F}~C_{M}U_{F12}~U_{M12}G_{F}~A_{M}G_{F}~A_{M}U_{F12}~G_{M}~A_{F}~U_{M}~C_{F4}~C_{M}~A_{M}~A_{M}
P-U_{M}~U_{F}~A_{M}A_{F}~G_{M}A_{F}~C_{M}U_{F12}~U_{M12}G_{F}~A_{M}G_{F}~A_{M}U_{F}~G_{M}~A_{F}~U_{M12}~C_{F}~C_{M4}~A_{M}~A_{M}
P-U_{M}~U_{F12}~A_{M}A_{F}~G_{M}A_{F}~C_{M}U_{F}~U_{M12}G_{F}~A_{M}G_{F}~A_{M}U_{F}~G_{M}~A_{F}~U_{M}~C_{F4}~C_{M}~A_{M}~A_{M}
P-U_{M}~U_{F12}~A_{M}A_{F}~G_{M}A_{F}~C_{M4}U_{F12}~U_{M}G_{F}~A_{M}G_{F}~A_{M}U_{F12}~G_{M}~A_{F}~U_{M12}~C_{F}~C_{M4}~A_{M}~A_{M}
P-U_{M}~U_{F12}~A_{M}A_{F}~G_{M}A_{F}~C_{M4}U_{F}~U_{M12}G_{F}~A_{M}G_{F}~A_{M}U_{F12}~G_{M}~A_{F}~U_{M}~C_{F4}~C_{M}~A_{M}~A_{M}
P-U_{M}~U_{F12}~A_{M}A_{F}~G_{M}A_{F}~C_{M4}U_{F12}~U_{M12}G_{F}~A_{M}G_{F}~A_{M}U_{F12}~G_{M}~A_{F}~U_{M}~C_{F4}~C_{M4}~A_{M}~A_{M}
P-U_{M}~U_{F}~A_{M}A_{F}~G_{M}A_{F}~C_{M4}U_{F12}~U_{M}G_{F}~A_{M}G_{F}~A_{M}U~G_{M}~A_{F}~U_{M12}~C_{F}~C_{M4}~A_{M}~A_{M}
P-U_{M}~U_{F}~A_{M}A_{F}~G_{M}A_{F}~C_{M4}U_{F12}~U_{M}G_{F}~A_{M}G_{F}~A_{M}U~G_{M}~A_{F}~U_{M12}~C_{F4}~C_{M}~A_{M}~A_{M}
P-U_{M}~U_{F}~A_{M}A_{F}~G_{M}A_{F}~C_{M}U_{F12}~U_{M12}G_{F}~A_{M}G_{F}~A_{M}U~G_{M}~A_{F}~U_{M}~C_{F4}~C_{M}~A_{M}~A_{M}
P-U_{M}~U_{F}~A_{M}A_{F}~G_{M}A_{F}~C_{M}U_{F12}~U_{M12}G_{F}~A_{M}G_{F}~A_{M}U~G_{M}~A_{F}~U_{M12}~C_{F}~C_{M4}~A_{M}~A_{M}
P-U_{M}~U_{F12}~A_{M}A_{F}~G_{M}A_{F}~C_{M}U_{F}~U_{M12}G_{F}~A_{M}G_{F}~A_{M}U~G_{M}~A_{F}~U_{M}~C_{F4}~C_{M}~A_{M}~A_{M}
P-U_{M}~U_{F12}~A_{M}A_{F}~G_{M}A_{F}~C_{M4}U_{F12}~U_{M}G_{F}~A_{M}G_{F}~A_{M}U~G_{M}~A_{F}~U_{M12}~C_{F}~C_{M4}~A_{M}~A_{M}
P-U_{M}~U_{F12}~A_{M}A_{F}~G_{M}A_{F}~C_{M4}U_{F}~U_{M12}G_{F}~A_{M}G_{F}~A_{M}U~G_{M}~A_{F}~U_{M}~C_{F4}~C_{M}~A_{M}~A_{M}
P-U_{M}~U_{F12}~A_{M}A_{F}~G_{M}A_{F}~C_{M4}U_{F12}~U_{M12}G_{F}~A_{M}G_{F}~A_{M}U~G_{M}~A_{F}~U_{M}~C_{F4}~C_{M4}~A_{M}~A_{M}
P-U_{M}~U_{F}~A_{M}A_{F}~G_{M}A_{F}~C_{M4}U_{F18}~U_{M}G_{F}~A_{M}G_{F}~A_{M}U_{F18}~G_{M}~A_{F}~U_{M18}~C_{F}~C_{M4}~A_{M}~A_{M}
P-U_{M}~U_{F}~A_{M}A_{F}~G_{M}A_{F}~C_{M4}U_{F18}~U_{M}G_{F}~A_{M}G_{F}~A_{M}U_{F18}~G_{M}~A_{F}~U_{M18}~C_{F4}~C_{M}~A_{M}~A_{M}
P-U_{M}~U_{F}~A_{M}A_{F}~G_{M}A_{F}~C_{M}U_{F18}~U_{M18}G_{F}~A_{M}G_{F}~A_{M}U_{F18}~G_{M}~A_{F}~U_{M}~C_{F4}~C_{M}~A_{M}~A_{M}
P-U_{M}~U_{F}~A_{M}A_{F}~G_{M}A_{F}~C_{M}U_{F18}~U_{M18}G_{F}~A_{M}G_{F}~A_{M}U_{F}~G_{M}~A_{F}~U_{M18}~C_{F}~C_{M4}~A_{M}~A_{M}
P-U_{M}~U_{F18}~A_{M}A_{F}~G_{M}A_{F}~C_{M}U_{F}~U_{M18}G_{F}~A_{M}G_{F}~A_{M}U_{F}~G_{M}~A_{F}~U_{M}~C_{F4}~C_{M}~A_{M}~A_{M}
P-U_{M}~U_{F18}~A_{M}A_{F}~G_{M}A_{F}~C_{M4}U_{F18}~U_{M}G_{F}~A_{M}G_{F}~A_{M}U_{F18}~G_{M}~A_{F}~U_{M18}~C_{F}~C_{M4}~A_{M}~A_{M}
P-U_{M}~U_{F18}~A_{M}A_{F}~G_{M}A_{F}~C_{M4}U_{F}~U_{M18}G_{F}~A_{M}G_{F}~A_{M}U_{F18}~G_{M}~A_{F}~U_{M}~C_{F4}~C_{M}~A_{M}~A_{M}
P-U_{M}~U_{F18}~A_{M}A_{F}~G_{M}A_{F}~C_{M4}U_{F18}~U_{M1s}G_{F}~A_{M}G_{F}~A_{M}U_{F18}~G_{M}~A_{F}~U_{M}~C_{F4}~C_{M4}~A_{M}~A_{M}
P-U_{M}~U_{F}~A_{M}A_{F}~G_{M}A_{F}~C_{M4}U_{F18}~U_{M}G_{F}~A_{M}G_{F}~A_{M}U~G_{M}~A_{F}~U_{M18}~C_{F}~C_{M4}~A_{M}~A_{M}
P-U_{M}~U_{F}~A_{M}A_{F}~G_{M}A_{F}~C_{M4}U_{F18}~U_{M}G_{F}~A_{M}G_{F}~A_{M}U~G_{M}~A_{F}~U_{M18}~C_{F4}~C_{M}~A_{M}~A_{M}
P-U_{M}~U_{F}~A_{M}A_{F}~G_{M}A_{F}~C_{M}U_{F18}~U_{M18}G_{F}~A_{M}G_{F}~A_{M}U~G_{M}~A_{F}~U_{M}~C_{F4}~C_{M}~A_{M}~A_{M}
P-U_{M}~U_{F}~A_{M}A_{F}~G_{M}A_{F}~C_{M}U_{F18}~U_{M18}G_{F}~A_{M}G_{F}~A_{M}U~G_{M}~A_{F}~U_{M18}~C_{F}~C_{M4}~A_{M}~A_{M}
P-U_{M}~U_{F18}~A_{M}A_{F}~G_{M}A_{F}~C_{M}U_{F}~U_{M18}G_{F}~A_{M}G_{F}~A_{M}U~G_{M}~A_{F}~U_{M}~C_{F4}~C_{M}~A_{M}~A_{M}
P-U_{M}~U_{F18}~A_{M}A_{F}~G_{M}A_{F}~C_{M4}U_{F18}~U_{M}G_{F}~A_{M}G_{F}~A_{M}U~G_{M}~A_{F}~U_{M18}~C_{F}~C_{M4}~A_{M}~A_{M}
P-U_{M}~U_{F18}~A_{M}A_{F}~G_{M}A_{F}~C_{M4}U_{F}~U_{M18}G_{F}~A_{M}G_{F}~A_{M}U~G_{M}~A_{F}~U_{M}~C_{F4}~C_{M}~A_{M}~A_{M}
P-U_{M}~U_{F18}~A_{M}A_{F}~G_{M}A_{F}~C_{M4}U_{F18}~U_{M18}G_{F}~A_{M}G_{F}~A_{M}U~G_{M}~A_{F}~U_{M}~C_{F4}~C_{M4}~A_{M}~A_{M}
P-U_{M}~U_{F}~A_{M}A_{L}~G_{M}A_{F}~C_{M}U_{L}~U_{M}G_{F}~A_{M}G_{L}~A_{M}U_{F}~G_{L}~A_{F}~U_{M}~C_{L}~C_{M}~A_{M}~A_{M}
P-U_{M}~U_{F}~A_{M}A_{L}~G_{M}A_{F}~C_{M}U_{L}~U_{M}G_{F}~A_{M}G_{L}~A_{M}U~G_{L}~A_{F}~U_{M}~C_{L}~C_{M}~A_{M}~A_{M}
P-U_{M}~U_{F}~A_{M}A_{L}~G_{M}A_{F}~C_{M4}U_{L3}~U_{M}G_{F}~A_{M}G_{L}~A_{M}U_{F3}~G_{L}~A_{F}~U_{M3}~C_{L}~C_{M4}~A_{M}~A_{M}
P-U_{M}~U_{F}~A_{M}A_{L}~G_{M}A_{F}~C_{M4}U_{L3}~U_{M}G_{F}~A_{M}G_{L}~A_{M}U_{F3}~G_{L}~A_{F}~U_{M3}~C_{L4}~C_{M}~A_{M}~A_{M}
P-U_{M}~U_{F}~A_{M}A_{L}~G_{M}A_{F}~C_{M}U_{L3}~U_{M3}G_{F}~A_{M}G_{L}~A_{M}U_{F3}~G_{L}~A_{F}~U_{M}~C_{L4}~C_{M}~A_{M}~A_{M}
P-U_{M}~U_{F}~A_{M}A_{L}~G_{M}A_{F}~C_{M}U_{L3}~U_{M3}G_{F}~A_{M}G_{L}~A_{M}U_{F}~G_{L}~A_{F}~U_{M3}~C_{L}~C_{M4}~A_{M}~A_{M}
P-U_{M}~U_{F3}~A_{M}A_{L}~G_{M}A_{F}~C_{M}U_{L}~U_{M3}G_{F}~A_{M}G_{L}~A_{M}U_{F}~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{L3}~U_{M}G_{F}~A_{M}G_{L}~A_{M}U_{F3}~G_{L}~A_{F}~U_{M3}~C_{L}~C_{M4}~A_{M}~A_{M}
P-U_{M}~U_{F3}~A_{M}A_{L}~G_{M}A_{F}~C_{M4}U_{L}~U_{M3}G_{F}~A_{M}G_{L}~A_{M}U_{F3}~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{L3}~U_{M3}G_{F}~A_{M}G_{L}~A_{M}U_{F3}~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{L3}~U_{M}G_{F}~A_{M}G_{L}~A_{M}U~G_{L}~A_{F}~U_{M3}~C_{L}~C_{M4}~A_{M}~A_{M}
P-U_{M}~U_{F}~A_{M}A_{L}~G_{M}A_{F}~C_{M4}U_{L3}~U_{M}G_{F}~A_{M}G_{L}~A_{M}U~G_{L}~A_{F}~U_{M3}~C_{L4}~C_{M}~A_{M}~A_{M}
P-U_{M}~U_{F}~A_{M}A_{L}~G_{M}A_{F}~C_{M}U_{L3}~U_{M3}G_{F}~A_{M}G_{L}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{M}U_{L3}~U_{M3}G_{F}~A_{M}G_{L}~A_{M}U~G_{L}~A_{F}~U_{M3}~C_{L}~C_{M4}~A_{M}~A_{M}
P-U_{M}~U_{F3}~A_{M}A_{L}~G_{M}A_{F}~C_{M}U_{L}~U_{M3}G_{F}~A_{M}G_{L}~A_{M}U~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{L3}~U_{M}G_{F}~A_{M}G_{L}~A_{M}U~G_{L}~A_{F}~U_{M3}~C_{L}~C_{M4}~A_{M}~A_{M}
P-U_{M}~U_{F3}~A_{M}A_{L}~G_{M}A_{F}~C_{M4}U_{L}~U_{M3}G_{F}~A_{M}G_{L}~A_{M}U~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{L3}~U_{M3}G_{F}~A_{M}G_{L}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{L12}~U_{M}G_{F}~A_{M}G_{L}~A_{M}U_{F12}~G_{L}~A_{F}~U_{M12}~C_{L}~C_{M4}~A_{M}~A_{M}
P-U_{M}~U_{F}~A_{M}A_{L}~G_{M}A_{F}~C_{M4}U_{L12}~U_{M}G_{F}~A_{M}G_{L}~A_{M}U_{F12}~G_{L}~A_{F}~U_{M12}~C_{L4}~C_{M}~A_{M}~A_{M}
P-U_{M}~U_{F}~A_{M}A_{L}~G_{M}A_{F}~C_{M}U_{L12}~U_{M12}G_{F}~A_{M}G_{L}~A_{M}U_{F12}~G_{L}~A_{F}~U_{M}~C_{L4}~C_{M}~A_{M}~A_{M}
P-U_{M}~U_{F}~A_{M}A_{L}~G_{M}A_{F}~C_{M}U_{L12}~U_{M12}G_{F}~A_{M}G_{L}~A_{M}U_{F}~G_{L}~A_{F}~U_{M12}~C_{L}~C_{M4}~A_{M}~A_{M}
P-U_{M}~U_{F12}~A_{M}A_{L}~G_{M}A_{F}~C_{M}U_{L}~U_{M12}G_{F}~A_{M}G_{L}~A_{M}U_{F}~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{L12}~U_{M}G_{F}~A_{M}G_{L}~A_{M}U_{F12}~G_{L}~A_{F}~U_{M12}~C_{L}~C_{M4}~A_{M}~A_{M}
P-U_{M}~U_{F12}~A_{M}A_{L}~G_{M}A_{F}~C_{M4}U_{L}~U_{M12}G_{F}~A_{M}G₁~A_{M}U_{F12}~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{L12}~U_{M12}G_{F}~A_{M}G_{L}~A_{M}U_{F12}~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{L12}~U_{M}G_{F}~A_{M}G_{L}~A_{M}U~G_{L}~A_{F}~U_{M12}~C_{L}~C_{M4}~A_{M}~A_{M}
P-U_{M}~U_{F}~A_{M}A_{L}~G_{M}A_{F}~C_{M4}U_{L12}~U_{M}G_{F}~A_{M}G_{L}~A_{M}U~G_{L}~A_{F}~U_{M12}~C_{L4}~C_{M}~A_{M}~A_{M}
P-U_{M}~U_{F}~A_{M}A_{L}~G_{M}A_{F}~C_{M}U_{L12}~U_{M12}G_{F}~A_{M}G_{L}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{M}U_{L12}~U_{M12}G_{F}~A_{M}G_{L}~A_{M}U~G_{L}~A_{F}~U_{M12}~C_{L}~C_{M4}~A_{M}~A_{M}
P-U_{M}~U_{F12}~A_{M}A_{L}~G_{M}A_{F}~C_{M}U_{L}~U_{M12}G_{F}~A_{M}G_{L}~A_{M}U~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{L12}~U_{M}G_{F}~A_{M}G_{L}~A_{M}U~G_{L}~A_{F}~U_{M12}~C_{L}~C_{M4}~A_{M}~A_{M}
P-U_{M}~U_{F12}~A_{M}A_{L}~G_{M}A_{F}~C_{M4}U_{L}~U_{M12}G_{F}~A_{M}G_{L}~A_{M}U~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{L12}~U_{M12}G_{F}~A_{M}G_{L}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{L18}~U_{M}G_{F}~A_{M}G_{L}~A_{M}U_{F18}~G_{L}~A_{F}~U_{M18}~C_{L}~C_{M4}~A_{M}~A_{M}
P-U_{M}~U_{F}~A_{M}A_{L}~G_{M}A_{F}~C_{M4}U_{L18}~U_{M}G_{F}~A_{M}G_{L}~A_{M}U_{F18}~G_{L}~A_{F}~U_{M18}~C_{L4}~C_{M}~A_{M}~A_{M}
P-U_{M}~U_{F}~A_{M}A_{L}~G_{M}A_{F}~C_{M}U_{L18}~U_{M18}G_{F}~A_{M}G_{L}~A_{M}U_{F18}~G_{L}~A_{F}~U_{M}~C_{L4}~C_{M}~A_{M}~A_{M}
P-U_{M}~U_{F}~A_{M}A_{L}~G_{M}A_{F}~C_{M}U_{L18}~U_{M18}G_{F}~A_{M}G_{L}~A_{M}U_{F}~G_{L}~A_{F}~U_{M18}~C_{L}~C_{M4}~A_{M}~A_{M}
P-U_{M}~U_{F18}~A_{M}A_{L}~G_{M}A_{F}~C_{M}U_{L}~U_{M18}G_{F}~A_{M}G_{L}~A_{M}U_{F}~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{L18}~U_{M}G_{F}~A_{M}G_{L}~A_{M}U_{F18}~G_{L}~A_{F}~U_{M18}~C_{L}~C_{M4}~A_{M}~A_{M}
P-U_{M}~U_{F18}~A_{M}A_{L}~G_{M}A_{F}~C_{M4}U_{L}~U_{M18}G_{F}~A_{M}G_{L}~A_{M}U_{F18}~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{L18}~U_{M18}G_{F}~A_{M}G_{L}~A_{M}U_{F18}~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{L18}~U_{M}G_{F}~A_{M}G_{L}~A_{M}U~G_{L}~A_{F}~U_{M18}~C_{L}~C_{M4}~A_{M}~A_{M}
P-U_{M}~U_{F}~A_{M}A_{L}~G_{M}A_{F}~C_{M4}U_{L18}~U_{M}G_{F}~A_{M}G_{L}~A_{M}U~G_{L}~A_{F}~U_{M18}~C_{L4}~C_{M}~A_{M}~A_{M}
P-U_{M}~U_{F}~A_{M}A_{L}~G_{M}A_{F}~C_{M}U_{L18}~U_{M18}G_{F}~A_{M}G_{L}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{M}U_{L18}~U_{M18}G_{F}~A_{M}G_{L}~A_{M}U~G_{L}~A_{F}~U_{M18}~C_{L}~C_{M4}~A_{M}~A_{M}
P-U_{M}~U_{F18}~A_{M}A_{L}~G_{M}A_{F}~C_{M}U_{L}~U_{M18}G_{F}~A_{M}G_{L}~A_{M}U~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{L18}~U_{M}G_{F}~A_{M}G_{L}~A_{M}U~G_{L}~A_{F}~U_{M18}~C_{L}~C_{M4}~A_{M}~A_{M}
P-U_{M}~U_{F18}~A_{M}A_{L}~G_{M}A_{F}~C_{M4}U_{L}~U_{M18}G_{F}~A_{M}G_{L}~A_{M}U~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{L18}~U_{M18}G_{F}~A_{M}G_{L}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{L}U_{F}~U_{M}G_{L}~A_{M}G_{F}~A_{M}U_{F}~G_{L}~A_{F}~U_{M}~C_{L}~C_{M}~A_{M}~A_{M}
P-U_{M}~U_{F}~A_{M}A_{L}~G_{M}A_{F}~C_{L}U_{F}~U_{M}G_{L}~A_{M}G_{F}~A_{M}U~G_{L}~A_{F}~U_{M}~C_{L}~C_{M}~A_{M}~A_{M}
P-U_{M}~U_{F}~A_{M}A_{L}~G_{M}A_{F}~C_{L4}U_{F3}~U_{M}G_{L}~A_{M}G_{F}~A_{M}U_{F3}~G_{L}~A_{F}~U_{M3}~C_{L}~C_{M4}~A_{M}~A_{M}
P-U_{M}~U_{F}~A_{M}A_{L}~G_{M}A_{F}~C_{L4}U_{F3}~U_{M}G_{L}~A_{M}G_{F}~A_{M}U_{F3}~G_{L}~A_{F}~U_{M3}~C_{L4}~C_{M}~A_{M}~A_{M}
P-U_{M}~U_{F}~A_{M}A_{L}~G_{M}A_{F}~C_{L}U_{F3}~U_{M3}G_{L}~A_{M}G_{F}~A_{M}U_{F3}~G_{L}~A_{F}~U_{M}~C_{L4}~C_{M}~A_{M}~A_{M}
P-U_{M}~U_{F}~A_{M}A_{L}~G_{M}A_{F}~C_{L}U_{F3}~U_{M3}G_{L}~A_{M}G_{F}~A_{M}U_{F}~G_{L}~A_{F}~U_{M3}~C_{L}~C_{M4}~A_{M}~A_{M}
P-U_{M}~U_{F3}~A_{M}A_{L}~G_{M}A_{F}~C_{L}U_{F}~U_{M3}G_{L}~A_{M}G_{F}~A_{M}U_{F}~G_{L}~A_{F}~U_{M}~C_{L4}~C_{M}~A_{M}~A_{M}
P-U_{M}~U_{F3}~A_{M}A_{L}~G_{M}A_{F}~C_{L4}U_{F3}~U_{M}G_{L}~A_{M}G_{F}~A_{M}U_{F3}~G_{L}~A_{F}~U_{M3}~C_{L}~C_{M4}~A_{M}~A_{M}
P-U_{M}~U_{F3}~A_{M}A_{L}~G_{M}A_{F}~C_{L4}U_{F}~U_{M3}G_{L}~A_{M}G_{F}~A_{M}U_{F3}~G_{L}~A_{F}~U_{M}~C_{L4}~C_{M}~A_{M}~A_{M}
P-U_{M}~U_{F3}~A_{M}A_{L}~G_{M}A_{F}~C_{L4}U_{F3}~U_{M3}G_{L}~A_{M}G_{F}~A_{M}U_{F3}~G_{L}~A_{F}~U_{M}~C_{L4}~C_{M4}~A_{M}~A_{M}
P-U_{M}~U_{F}~A_{M}A_{L}~G_{M}A_{F}~C_{L4}U_{F3}~U_{M}G_{L}~A_{M}G_{F}~A_{M}U~G_{L}~A_{F}~U_{M3}~C_{L}~C_{M4}~A_{M}~A_{M}
P-U_{M}~U_{F}~A_{M}A_{L}~G_{M}A_{F}~C_{L4}U_{F3}~U_{M}G_{L}~A_{M}G_{F}~A_{M}U~G_{L}~A_{F}~U_{M3}~C_{L4}~C_{M}~A_{M}~A_{M}
P-U_{M}~U_{F}~A_{M}A_{L}~G_{M}A_{F}~C_{L}U_{F3}~U_{M3}G_{L}~A_{M}G_{F}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{L}U_{F3}~U_{M3}G_{L}~A_{M}G_{F}~A_{M}U~G_{L}~A_{F}~U_{M3}~C_{L}~C_{M4}~A_{M}~A_{M}
P-U_{M}~U_{F3}~A_{M}A_{L}~G_{M}A_{F}~C_{L}U_{F}~U_{M3}G_{L}~A_{M}G_{F}~A_{M}U~G_{L}~A_{F}~U_{M}~C_{L4}~C_{M}~A_{M}~A_{M}
P-U_{M}~U_{F3}~A_{M}A_{L}~G_{M}A_{F}~C_{L4}U_{F3}~U_{M}G_{L}~A_{M}G_{F}~A_{M}U~G_{L}~A_{F}~U_{M3}~C_{L}~C_{M4}~A_{M}~A_{M}
P-U_{M}~U_{F3}~A_{M}A_{L}~G_{M}A_{F}~C_{L4}U_{F}~U_{M3}G_{L}~A_{M}G_{F}~A_{M}U~G_{L}~A_{F}~U_{M}~C_{L4}~C_{M}~A_{M}~A_{M}
P-U_{M}~U_{F3}~A_{M}A_{L}~G_{M}A_{F}~C_{L4}U_{F3}~U_{M3}G_{L}~A_{M}G_{F}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{L4}U_{F12}~U_{M}G_{L}~A_{M}G_{F}~A_{M}U_{F12}~G_{L}~A_{F}~U_{M12}~C_{L}~C_{M4}~A_{M}~A_{M}
P-U_{M}~U_{F}~A_{M}A_{L}~G_{M}A_{F}~C_{L4}U_{F12}~U_{M}G_{L}~A_{M}G_{F}~A_{M}U_{F12}~G_{L}~A_{F}~U_{M12}~C_{L4}~C_{M}~A_{M}~A_{M}
P-U_{M}~U_{F}~A_{M}A_{L}~G_{M}A_{F}~C_{L}U_{F12}~U_{M12}G_{L}~A_{M}G_{F}~A_{M}U_{F12}~G_{L}~A_{F}~U_{M}~C_{L4}~C_{M}~A_{M}~A_{M}
P-U_{M}~U_{F}~A_{M}A_{L}~G_{M}A_{F}~C_{L}U_{F12}~U_{M12}G_{L}~A_{M}G_{F}~A_{M}U_{F}~G_{L}~A_{F}~U_{M12}~C_{L}~C_{M4}~A_{M}~A_{M}
P-U_{M}~U_{F12}~A_{M}A_{L}~G_{M}A_{F}~C_{L}U_{F}~U_{M12}G_{L}~A_{M}G_{F}~A_{M}U_{F}~G_{L}~A_{F}~U_{M}~C_{L4}~C_{M}~A_{M}~A_{M}
P-U_{M}~U_{F12}~A_{M}A_{L}~G_{M}A_{F}~C_{L4}U_{F12}~U_{M}G_{L}~A_{M}G_{F}~A_{M}U_{F12}~G_{L}~A_{F}~U_{M12}~C_{L}~C_{M4}~A_{M}~A_{M}
P-U_{M}~U_{F12}~A_{M}A_{L}~G_{M}A_{F}~C_{L4}U_{F}~U_{M12}G_{L}~A_{M}G_{F}~A_{M}U_{F12}~G_{L}~A_{F}~U_{M}~C_{L4}~C_{M}~A_{M}~A_{M}
P-U_{M}~U_{F12}~A_{M}A_{L}~G_{M}A_{F}~C_{L4}U_{F12}~U_{M12}G_{L}~A_{M}G_{F}~A_{M}U_{F12}~G_{L}~A_{F}~U_{M}~C_{L4}~C_{M4}~A_{M}~A_{M}
P-U_{M}~U_{F}~A_{M}A_{L}~G_{M}A_{F}~C_{L4}U_{F12}~U_{M}G_{L}~A_{M}G_{F}~A_{M}U~G_{L}~A_{F}~U_{M12}~C_{L}~C_{M4}~A_{M}~A_{M}
P-U_{M}~U_{F}~A_{M}A_{L}~G_{M}A_{F}~C_{L4}U_{F12}~U_{M}G_{L}~A_{M}G_{F}~A_{M}U~G_{L}~A_{F}~U_{M12}~C_{L4}~C_{M}~A_{M}~A_{M}
P-U_{M}~U_{F}~A_{M}A_{L}~G_{M}A_{F}~C_{L}U_{F12}~U_{M12}G_{L}~A_{M}G_{F}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{L}U_{F12}~U_{M12}G_{L}~A_{M}G_{F}~A_{M}U~G_{L}~A_{F}~U_{M12}~C_{L}~C_{M4}~A_{M}~A_{M}
P-U_{M}~U_{F12}~A_{M}A_{L}~G_{M}A_{F}~C_{L}U_{F}~U_{M12}G_{L}~A_{M}G_{F}~A_{M}U~G_{L}~A_{F}~U_{M}~C_{L4}~C_{M}~A_{M}~A_{M}
P-U_{M}~U_{F12}~A_{M}A_{L}~G_{M}A_{F}~C_{L4}U_{F12}~U_{M}G_{L}~A_{M}G_{F}~A_{M}U~G_{L}~A_{F}~U_{M12}~C_{L}~C_{M4}~A_{M}~A_{M}
P-U_{M}~U_{F12}~A_{M}A_{L}~G_{M}A_{F}~C_{L4}U_{F}~U_{M12}G_{L}~A_{M}G_{F}~A_{M}U~G_{L}~A_{F}~U_{M}~C_{L4}~C_{M}~A_{M}~A_{M}
P-U_{M}~U_{F12}~A_{M}A_{L}~G_{M}A_{F}~C_{L4}U_{F12}~U_{M12}G_{L}~A_{M}G_{F}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{L4}U_{F18}~U_{M}G_{L}~A_{M}G_{F}~A_{M}U_{F18}~G_{L}~A_{F}~U_{M18}~C_{L}~C_{M4}~A_{M}~A_{M}
P-U_{M}~U_{F}~A_{M}A_{L}~G_{M}A_{F}~C_{L4}U_{F18}~U_{M}G_{L}~A_{M}G_{F}~A_{M}U_{F18}~G_{L}~A_{F}~U_{M18}~C_{L4}~C_{M}~A_{M}~A_{M}
P-U_{M}~U_{F}~A_{M}A_{L}~G_{M}A_{F}~C_{L}U_{F18}~U_{M18}G_{L}~A_{M}G_{F}~A_{M}U_{F18}~G_{L}~A_{F}~U_{M}~C_{L4}~C_{M}~A_{M}~A_{M}
P-U_{M}~U_{F}~A_{M}A_{L}~G_{M}A_{F}~C_{L}U_{F18}~U_{M18}G_{L}~A_{M}G_{F}~A_{M}U_{F}~G_{L}~A_{F}~U_{M18}~C_{L}~C_{M4}~A_{M}~A_{M}
P-U_{M}~U_{F18}~A_{M}A_{L}~G_{M}A_{F}~C_{L}U_{F}~U_{M18}G_{L}~A_{M}G_{F}~A_{M}U_{F}~G_{L}~A_{F}~U_{M}~C_{L4}~C_{M}~A_{M}~A_{M}
P-U_{M}~U_{F18}~A_{M}A_{L}~G_{M}A_{F}~C_{L4}U_{F18}~U_{M}G_{L}~A_{M}G_{F}~A_{M}U_{F18}~G_{L}~A_{F}~U_{M18}~C_{L}~C_{M4}~A_{M}~A_{M}
P-U_{M}~U_{F18}~A_{M}A_{L}~G_{M}A_{F}~C_{L4}U_{F}~U_{M18}G_{L}~A_{M}G_{F}~A_{M}U_{F18}~G_{L}~A_{F}~U_{M}~C_{L4}~C_{M}~A_{M}~A_{M}
P-U_{M}~U_{F18}~A_{M}A_{L}~G_{M}A_{F}~C_{L4}U_{F18}~U_{M18}G_{L}~A_{M}G_{F}~A_{M}U_{F18}~G_{L}~A_{F}~U_{M}~C_{L4}~C_{M4}~A_{M}~A_{M}
P-U_{M}~U_{F}~A_{M}A_{L}~G_{M}A_{F}~C_{L4}U_{F18}~U_{M}G_{L}~A_{M}G_{F}~A_{M}U~G_{L}~A_{F}~U_{M18}~C_{L}~C_{M4}~A_{M}~A_{M}
P-U_{M}~U_{F}~A_{M}A_{L}~G_{M}A_{F}~C_{L4}U_{F18}~U_{M}G_{L}~A_{M}G_{F}~A_{M}U~G_{L}~A_{F}~U_{M18}~C_{L4}~C_{M}~A_{M}~A_{M}
P-U_{M}~U_{F}~A_{M}A_{L}~G_{M}A_{F}~C_{L}U_{F18}~U_{M18}G_{L}~A_{M}G_{F}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{L}U_{F18}~U_{M18}G_{L}~A_{M}G_{F}~A_{M}U~G_{L}~A_{F}~U_{M18}~C_{L}~C_{M4}~A_{M}~A_{M}
P-U_{M}~U_{F18}~A_{M}A_{L}~G_{M}A_{F}~C_{L}U_{F}~U_{M18}G_{L}~A_{M}G_{F}~A_{M}U~G_{L}~A_{F}~U_{M}~C_{L4}~C_{M}~A_{M}~A_{M}
P-U_{M}~U_{F18}~A_{M}A_{L}~G_{M}A_{F}~C_{L4}U_{F18}~U_{M}G_{L}~A_{M}G_{F}~A_{M}U~G_{L}~A_{F}~U_{M18}~C_{L}~C_{M4}~A_{M}~A_{M}
P-U_{M}~U_{F18}~A_{M}A_{L}~G_{M}A_{F}~C_{L4}U_{F}~U_{M18}G_{L}~A_{M}G_{F}~A_{M}U~G_{L}~A_{F}~U_{M}~C_{L4}~C_{M}~A_{M}~A_{M}
P-U_{M}~U_{F18}~A_{M}A_{L}~G_{M}A_{F}~C_{L4}U_{F18}~U_{M18}G_{L}~A_{M}G_{F}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{M}U_{F}~U_{M}G_{F}~A_{L}G_{F}~A_{M}U_{F}~G_{L}~A_{F}~U_{M}~C_{L}~C_{M}~A_{M}~A_{M}
P-U_{M}~U_{F}~A_{M}A_{L}~G_{M}A_{F}~C_{M}U_{F}~U_{M}G_{F}~A_{L}G_{F}~A_{M}U~G_{L}~A_{F}~U_{M}~C_{L}~C_{M}~A_{M}~A_{M}
P-U_{M}~U_{F}~A_{M}A_{L}~G_{M}A_{F}~C_{M4}U_{F3}~U_{M}G_{F}~A_{L}G_{F}~A_{M}U_{F3}~G_{L}~A_{F}~U_{M3}~C_{L}~C_{M4}~A_{M}~A_{M}
P-U_{M}~U_{F}~A_{M}A_{L}~G_{M}A_{F}~C_{M4}U_{F3}~U_{M}G_{F}~A_{L}G_{F}~A_{M}U_{F3}~G_{L}~A_{F}~U_{M3}~C_{L4}~C_{M}~A_{M}~A_{M}
P-U_{M}~U_{F}~A_{M}A_{L}~G_{M}A_{F}~C_{M}U_{F3}~U_{M3}G_{F}~A_{L}G_{F}~A_{M}U_{F3}~G_{L}~A_{F}~U_{M}~C_{L4}~C_{M}~A_{M}~A_{M}
P-U_{M}~U_{F}~A_{M}A_{L}~G_{M}A_{F}~C_{M}U_{F3}~U_{M3}G_{F}~A_{L}G_{F}~A_{M}U_{F}~G_{L}~A_{F}~U_{M3}~C_{L}~C_{M4}~A_{M}~A_{M}
P-U_{M}~U_{F3}~A_{M}A_{L}~G_{M}A_{F}~C_{M}U_{F}~U_{M3}G_{F}~A_{L}G_{F}~A_{M}U_{F}~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{F3}~U_{M}G_{F}~A_{L}G_{F}~A_{M}U_{F3}~G_{L}~A_{F}~U_{M3}~C_{L}~C_{M4}~A_{M}~A_{M}
P-U_{M}~U_{F3}~A_{M}A_{L}~G_{M}A_{F}~C_{M4}U_{F}~U_{M3}G_{F}~A_{L}G_{F}~A_{M}U_{F3}~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{F3}~U_{M3}G_{F}~A_{L}G_{F}~A_{M}U_{F3}~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{F3}~U_{M}G_{F}~A_{L}G_{F}~A_{M}U~G_{L}~A_{F}~U_{M3}~C_{L}~C_{M4}~A_{M}~A_{M}
P-U_{M}~U_{F}~A_{M}A_{L}~G_{M}A_{F}~C_{M4}U_{F3}~U_{M}G_{F}~A_{L}G_{F}~A_{M}U~G_{L}~A_{F}~U_{M3}~C_{L4}~C_{M}~A_{M}~A_{M}
P-U_{M}~U_{F}~A_{M}A_{L}~G_{M}A_{F}~C_{M}U_{F3}~U_{M3}G_{F}~A_{L}G_{F}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{M}U_{F3}~U_{M3}G_{F}~A_{L}G_{F}~A_{M}U~G_{L}~A_{F}~U_{M3}~C_{L}~C_{M4}~A_{M}~A_{M}
P-U_{M}~U_{F3}~A_{M}A_{L}~G_{M}A_{F}~C_{M}U_{F}~U_{M3}G_{F}~A_{L}G_{F}~A_{M}U~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{F3}~U_{M}G_{F}~A_{L}G_{F}~A_{M}U~G_{L}~A_{F}~U_{M3}~C_{L}~C_{M4}~A_{M}~A_{M}
P-U_{M}~U_{F3}~A_{M}A_{L}~G_{M}A_{F}~C_{M4}U_{F}~U_{M3}G_{F}~A_{L}G_{F}~A_{M}U~G_{L}~A_{F}~U_{M}~C_{L4}~C_{M}~A_{M}~A_{M}
P-U_{M}~U_{F3}~A_{M}A_{L}~G_{M}A_{F}~C_{M4}U_{F3}~U_{M3}G_{F}~A_{L}G_{F}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{F12}~U_{M}G_{F}~A_{L}G_{F}~A_{M}U_{F12}~G_{L}~A_{F}~U_{M12}~C_{L}~C_{M4}~A_{M}~A_{M}
P-U_{M}~U_{F}~A_{M}A_{L}~G_{M}A_{F}~C_{M4}U_{F12}~U_{M}G_{F}~A_{L}G_{F}~A_{M}U_{F12}~G_{L}~A_{F}~U_{M12}~C_{L4}~C_{M}~A_{M}~A_{M}
P-U_{M}~U_{F}~A_{M}A_{L}~G_{M}A_{F}~C_{M}U_{F12}~U_{M12}G_{F}~A_{L}G_{F}~A_{M}U_{F12}~G_{L}~A_{F}~U_{M}~C_{L4}~C_{M}~A_{M}~A_{M}
P-U_{M}~U_{F}~A_{M}A_{L}~G_{M}A_{F}~C_{M}U_{F12}~U_{M12}G_{F}~A_{L}G_{F}~A_{M}U_{F}~G_{L}~A_{F}~U_{M12}~C_{L}~C_{M4}~A_{M}~A_{M}
P-U_{M}~U_{F12}~A_{M}A_{L}~G_{M}A_{F}~C_{M}U_{F}~U_{M12}G_{F}~A_{L}G_{F}~A_{M}U_{F}~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{F12}~U_{M}G_{F}~A_{L}G_{F}~A_{M}U_{F12}~G_{L}~A_{F}~U_{M12}~C_{L}~C_{M4}~A_{M}~A_{M}
P-U_{M}~U_{F12}~A_{M}A_{L}~G_{M}A_{F}~C_{M4}U_{F}~U_{M12}G_{F}~A_{L}G_{F}~A_{M}U_{F12}~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{F12}~U_{M12}G_{F}~A_{L}G_{F}~A_{M}U_{F12}~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{F12}~U_{M}G_{F}~A_{L}G_{F}~A_{M}U~G_{L}~A_{F}~U_{M12}~C_{L}~C_{M4}~A_{M}~A_{M}
P-U_{M}~U_{F}~A_{M}A_{L}~G_{M}A_{F}~C_{M4}U_{F12}~U_{M}G_{F}~A_{L}G_{F}~A_{M}U~G_{L}~A_{F}~U_{M12}~C_{L4}~C_{M}~A_{M}~A_{M}
P-U_{M}~U_{F}~A_{M}A_{L}~G_{M}A_{F}~C_{M}U_{F12}~U_{M12}G_{F}~A_{L}G_{F}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{M}U_{F12}~U_{M12}G_{F}~A_{L}G_{F}~A_{M}U~G_{L}~A_{F}~U_{M12}~C_{L}~C_{M4}~A_{M}~A_{M}
P-U_{M}~U_{F12}~A_{M}A_{L}~G_{M}A_{F}~C_{M}U_{F}~U_{M12}G_{F}~A_{L}G_{F}~A_{M}U~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{F12}~U_{M}G_{F}~A_{L}G_{F}~A_{M}U~G_{L}~A_{F}~U_{M12}~C_{L}~C_{M4}~A_{M}~A_{M}
P-U_{M}~U_{F12}~A_{M}A_{L}~G_{M}A_{F}~C_{M4}U_{F}~U_{M12}G_{F}~A_{L}G_{F}~A_{M}U~G_{L}~A_{F}~U_{M}~C_{L4}~C_{M}~A_{M}~A_{M}
P-U_{M}~U_{F12}~A_{M}A_{L}~G_{M}A_{F}~C_{M4}U_{F12}~U_{M12}G_{F}~A_{L}G_{F}~A_{M}U~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{F18}~U_{M}G_{F}~A_{L}G_{F}~A_{M}U_{F18}~G_{L}~A_{F}~U_{M18}~C_{L}~C_{M4}~A_{M}~A_{M}
P-U_{M}~U_{F}~A_{M}A_{L}~G_{M}A_{F}~C_{M4}U_{F18}~U_{M}G_{F}~A_{L}G_{F}~A_{M}U_{F18}~G_{L}~A_{F}~U_{M18}~C_{L4}~C_{M}~A_{M}~A_{M}
P-U_{M}~U_{F}~A_{M}A_{L}~G_{M}A_{F}~C_{M}U_{F18}~U_{M18}G_{F}~A_{L}G_{F}~A_{M}U_{F18}~G_{L}~A_{F}~U_{M}~C_{L4}~C_{M}~A_{M}~A_{M}
P-U_{M}~U_{F}~A_{M}A_{L}~G_{M}A_{F}~C_{M}U_{F18}~U_{M18}G_{F}~A_{L}G_{F}~A_{M}U_{F}~G_{L}~A_{F}~U_{M18}~C_{L}~C_{M4}~A_{M}~A_{M}
P-U_{M}~U_{F18}~A_{M}A_{L}~G_{M}A_{F}~C_{M}U_{F}~U_{M18}G_{F}~A_{L}G_{F}~A_{M}U_{F}~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{F18}~U_{M}G_{F}~A_{L}G_{F}~A_{M}U_{F18}~G_{L}~A_{F}~U_{M18}~C_{L}~C_{M4}~A_{M}~A_{M}
P-U_{M}~U_{F18}~A_{M}A_{L}~G_{M}A_{F}~C_{M4}U_{F}~U_{M18}G_{F}~A_{L}G_{F}~A_{M}U_{F18}~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{F18}~U_{M18}G_{F}~A_{L}G_{F}~A_{M}U_{F18}~G_{L}~A_{F}~U_{M}~C_{L4}~C_{M4}~A_{M}~A_{M}
P-U_{M}~U_{F}~A_{M}A_{L}~G_{M}A_{F}~C_{M4}U_{F18}~U_{M}G_{F}~A_{L}G_{F}~A_{M}U~G_{L}~A_{F}~U_{M18}~C_{L}~C_{M4}~A_{M}~A_{M}
P-U_{M}~U_{F}~A_{M}A_{L}~G_{M}A_{F}~C_{M4}U_{F18}~U_{M}G_{F}~A_{L}G_{F}~A_{M}U~G_{L}~A_{F}~U_{M18}~C_{L4}~C_{M}~A_{M}~A_{M}
P-U_{M}~U_{F}~A_{M}A_{L}~G_{M}A_{F}~C_{M}U_{F18}~U_{M18}G_{F}~A_{L}G_{F}~A_{M}U~G_{L}~A_{F}~U_{M}~C_{L4}~C_{M}~A_{M}~A_{M}
P-U_{M}~U_{F}~A_{M}A_{L}~G_{M}A_{F}~C_{M}U_{F18}~U_{M18}G_{F}~A_{L}G_{F}~A_{M}U~G_{L}~A_{F}~U_{M18}~C_{L}~C_{M4}~A_{M}~A_{M}
P-U_{M}~U_{F18}~A_{M}A_{L}~G_{M}A_{F}~C_{M}U_{F}~U_{M18}G_{F}~A_{L}G_{F}~A_{M}U~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{F18}~U_{M}G_{F}~A_{L}G_{F}~A_{M}U~G_{L}~A_{F}~U_{M18}~C_{L}~C_{M4}~A_{M}~A_{M}
P-U_{M}~U_{F18}~A_{M}A_{L}~G_{M}A_{F}~C_{M4}U_{F}~U_{M18}G_{F}~A_{L}G_{F}~A_{M}U~G_{L}~A_{F}~U_{M}~C_{L4}~C_{M}~A_{M}~A_{M}
P-U_{M}~U_{F18}~A_{M}A_{L}~G_{M}A_{F}~C_{M4}U_{F18}~U_{M18}G_{F}~A_{L}G_{F}~A_{M}U~G_{L}~A_{F}~U_{M}~C_{L4}~C_{M4}~A_{M}~A_{M}
P-U_{M}~U_{F}~A_{L}A_{F}~G_{M}A_{L}~C_{M}U_{F}~U_{M}G_{F}~A_{M}G_{L}~A_{M}U_{F}~G_{M}~A_{F}~U_{L}~C_{F}~C_{M}~A_{M}~A_{M}
P-U_{M}~U_{F}~A_{L}A_{F}~G_{M}A_{L}~C_{M}U_{F}~U_{M}G_{F}~A_{M}G_{L}~A_{M}U~G_{M}~A_{F}~U_{L}~C_{F}~C_{M}~A_{M}~A_{M}
P-U_{M}~U_{F}~A_{L}A_{F}~G_{M}A_{L}~C_{M4}U_{F3}~U_{M}G_{F}~A_{M}G_{L}~A_{M}U_{F3}~G_{M}~A_{F}~U_{L3}~C_{F}~C_{M4}~A_{M}~A_{M}
P-U_{M}~U_{F}~A_{L}A_{F}~G_{M}A_{L}~C_{M4}U_{F3}~U_{M}G_{F}~A_{M}G_{L}~A_{M}U_{F3}~G_{M}~A_{F}~U_{L3}~C_{F4}~C_{M}~A_{M}~A_{M}
P-U_{M}~U_{F}~A_{L}A_{F}~G_{M}A_{L}~C_{M}U_{F3}~U_{M3}G_{F}~A_{M}G_{L}~A_{M}U_{F3}~G_{M}~A_{F}~U_{L}~C_{F4}~C_{M}~A_{M}~A_{M}
P-U_{M}~U_{F}~A_{L}A_{F}~G_{M}A_{L}~C_{M}U_{F3}~U_{M3}G_{F}~A_{M}G_{L}~A_{M}U_{F}~G_{M}~A_{F}~U_{L3}~C_{F}~C_{M4}~A_{M}~A_{M}
P-U_{M}~U_{F3}~A_{L}A_{F}~G_{M}A_{L}~C_{M}U_{F}~U_{M3}G_{F}~A_{M}G_{L}~A_{M}U_{F}~G_{M}~A_{F}~U_{L}~C_{F4}~C_{M}~A_{M}~A_{M}
P-U_{M}~U_{F3}~A_{L}A_{F}~G_{M}A_{L}~C_{M4}U_{F3}~U_{M}G_{F}~A_{M}G_{L}~A_{M}U_{F3}~G_{M}~A_{F}~U_{L3}~C_{F}~C_{M4}~A_{M}~A_{M}
P-U_{M}~U_{F3}~A_{L}A_{F}~G_{M}A_{L}~C_{M4}U_{F}~U_{M3}G_{F}~A_{M}G_{L}~A_{M}U_{F3}~G_{M}~A_{F}~U_{L}~C_{F4}~C_{M}~A_{M}~A_{M}
P-U_{M}~U_{F3}~A_{L}A_{F}~G_{M}A_{L}~C_{M4}U_{F3}~U_{M3}G_{F}~A_{M}G_{L}~A_{M}U_{F3}~G_{M}~A_{F}~U_{L}~C_{F4}~C_{M4}~A_{M}~A_{M}
P-U_{M}~U_{F}~A_{L}A_{F}~G_{M}A_{L}~C_{M4}U_{F3}~U_{M}G_{F}~A_{M}G_{L}~A_{M}U~G_{M}~A_{F}~U_{L3}~C_{F}~C_{M4}~A_{M}~A_{M}
P-U_{M}~U_{F}~A_{L}A_{F}~G_{M}A_{L}~C_{M4}U_{F3}~U_{M}G_{F}~A_{M}G_{L}~A_{M}U~G_{M}~A_{F}~U_{L3}~C_{F4}~C_{M}~A_{M}~A_{M}
P-U_{M}~U_{F}~A_{L}A_{F}~G_{M}A_{L}~C_{M}U_{F3}~U_{M3}G_{F}~A_{M}G_{L}~A_{M}U~G_{M}~A_{F}~U_{L}~C_{F4}~C_{M}~A_{M}~A_{M}
P-U_{M}~U_{F}~A_{L}A_{F}~G_{M}A_{L}~C_{M}U_{F3}~U_{M3}G_{F}~A_{M}G_{L}~A_{M}U~G_{M}~A_{F}~U_{L3}~C_{F}~C_{M4}~A_{M}~A_{M}
P-U_{M}~U_{F3}~A_{L}A_{F}~G_{M}A_{L}~C_{M}U_{F}~U_{M3}G_{F}~A_{M}G_{L}~A_{M}U~G_{M}~A_{F}~U_{L}~C_{F4}~C_{M}~A_{M}~A_{M}
P-U_{M}~U_{F3}~A_{L}A_{F}~G_{M}A_{L}~C_{M4}U_{F3}~U_{M}G_{F}~A_{M}G_{L}~A_{M}U~G_{M}~A_{F}~U_{L3}~C_{F}~C_{M4}~A_{M}~A_{M}
P-U_{M}~U_{F3}~A_{L}A_{F}~G_{M}A_{L}~C_{M4}U_{F}~U_{M3}G_{F}~A_{M}G_{L}~A_{M}U~G_{M}~A_{F}~U_{L}~C_{F4}~C_{M}~A_{M}~A_{M}
P-U_{M}~U_{F3}~A_{L}A_{F}~G_{M}A_{L}~C_{M4}U_{F3}~U_{M3}G_{F}~A_{M}G_{L}~A_{M}U~G_{M}~A_{F}~U_{L}~C_{F4}~C_{M4}~A_{M}~A_{M}
P-U_{M}~U_{F}~A_{L}A_{F}~G_{M}A_{L}~C_{M4}U_{F12}~U_{M}G_{F}~A_{M}G_{L}~A_{M}U_{F12}~G_{M}~A_{F}~U_{L12}~C_{F}~C_{M4}~A_{M}~A_{M}
P-U_{M}~U_{F}~A_{L}A_{F}~G_{M}A_{L}~C_{M4}U_{F12}~U_{M}G_{F}~A_{M}G_{L}~A_{M}U_{F12}~G_{M}~A_{F}~U_{L12}~C_{F4}~C_{M}~A_{M}~A_{M}
P-U_{M}~U_{F}~A_{L}A_{F}~G_{M}A_{L}~C_{M}U_{F12}~U_{M12}G_{F}~A_{M}G_{L}~A_{M}U_{F12}~G_{M}~A_{F}~U_{L}~C_{F4}~C_{M}~A_{M}~A_{M}
P-U_{M}~U_{F}~A_{L}A_{F}~G_{M}A_{L}~C_{M}U_{F12}~U_{M12}G_{F}~A_{M}G_{L}~A_{M}U_{F}~G_{M}~A_{F}~U_{L12}~C_{F}~C_{M4}~A_{M}~A_{M}
P-U_{M}~U_{F12}~A_{L}A_{F}~G_{M}A_{L}~C_{M}U_{F}~U_{M12}G_{F}~A_{M}G_{L}~A_{M}U_{F}~G_{M}~A_{F}~U_{L}~C_{F4}~C_{M}~A_{M}~A_{M}
P-U_{M}~U_{F12}~A_{L}A_{F}~G_{M}A_{L}~C_{M4}U_{F12}~U_{M}G_{F}~A_{M}G_{L}~A_{M}U_{F12}~G_{M}~A_{F}~U_{L12}~C_{F}~C_{M4}~A_{M}~A_{M}
P-U_{M}~U_{F12}~A_{L}A_{F}~G_{M}A_{L}~C_{M4}U_{F}~U_{M12}G_{F}~A_{M}G_{L}~A_{M}U_{F12}~G_{M}~A_{F}~U_{L}~C_{F4}~C_{M}~A_{M}~A_{M}
P-U_{M}~U_{F12}~A_{L}A_{F}~G_{M}A_{L}~C_{M4}U_{F12}~U_{M12}G_{F}~A_{M}G_{L}~A_{M}U_{F12}~G_{M}~A_{F}~U_{L}~C_{F4}~C_{M4}~A_{M}~A_{M}
P-U_{M}~U_{F}~A_{L}A_{F}~G_{M}A_{L}~C_{M4}U_{F12}~U_{M}G_{F}~A_{M}G_{L}~A_{M}U~G_{M}~A_{F}~U_{L12}~C_{F}~C_{M4}~A_{M}~A_{M}
P-U_{M}~U_{F}~A_{L}A_{F}~G_{M}A_{L}~C_{M4}U_{F12}~U_{M}G_{F}~A_{M}G_{L}~A_{M}U~G_{M}~A_{F}~U_{L12}~C_{F4}~C_{M}~A_{M}~A_{M}
P-U_{M}~U_{F}~A_{L}A_{F}~G_{M}A_{L}~C_{M}U_{F12}~U_{M12}G_{F}~A_{M}G_{L}~A_{M}U~G_{M}~A_{F}~U_{L}~C_{F4}~C_{M}~A_{M}~A_{M}
P-U_{M}~U_{F}~A_{L}A_{F}~G_{M}A_{L}~C_{M}U_{F12}~U_{M12}G_{F}~A_{M}G_{L}~A_{M}U~G_{M}~A_{F}~U_{L12}~C_{F}~C_{M4}~A_{M}~A_{M}
P-U_{M}~U_{F12}~A_{L}A_{F}~G_{M}A_{L}~C_{M}U_{F}~U_{M12}G_{F}~A_{M}G_{L}~A_{M}U~G_{M}~A_{F}~U_{L}~C_{F4}~C_{M}~A_{M}~A_{M}
P-U_{M}~U_{F12}~A_{L}A_{F}~G_{M}A_{L}~C_{M4}U_{F12}~U_{M}G_{F}~A_{M}G_{L}~A_{M}U~G_{M}~A_{F}~U_{L12}~C_{F}~C_{M4}~A_{M}~A_{M}
P-U_{M}~U_{F12}~A_{L}A_{F}~G_{M}A_{L}~C_{M4}U_{F}~U_{M12}G_{F}~A_{M}G_{L}~A_{M}U~G_{M}~A_{F}~U_{L}~C_{F4}~C_{M}~A_{M}~A_{M}
P-U_{M}~U_{F12}~A_{L}A_{F}~G_{M}A_{L}~C_{M4}U_{F12}~U_{M12}G_{F}~A_{M}G_{L}~A_{M}U~G_{M}~A_{F}~U_{L}~C_{F4}~C_{M4}~A_{M}~A_{M}
P-U_{M}~U_{F}~A_{L}A_{F}~G_{M}A_{L}~C_{M4}U_{F18}~U_{M}G_{F}~A_{M}G_{L}~A_{M}U_{F18}~G_{M}~A_{F}~U_{L18}~C_{F}~C_{M4}~A_{M}~A_{M}
P-U_{M}~U_{F}~A_{L}A_{F}~G_{M}A_{L}~C_{M4}U_{F18}~U_{M}G_{F}~A_{M}G_{L}~A_{M}U_{F18}~G_{M}~A_{F}~U_{L18}~C_{F4}~C_{M}~A_{M}~A_{M}
P-U_{M}~U_{F}~A_{L}A_{F}~G_{M}A_{L}~C_{M}U_{F18}~U_{M18}G_{F}~A_{M}G_{L}~A_{M}U_{F18}~G_{M}~A_{F}~U_{L}~C_{F4}~C_{M}~A_{M}~A_{M}
P-U_{M}~U_{F}~A_{L}A_{F}~G_{M}A_{L}~C_{M}U_{F18}~U_{M18}G_{F}~A_{M}G_{L}~A_{M}U_{F}~G_{M}~A_{F}~U_{L18}~C_{F}~C_{M4}~A_{M}~A_{M}
P-U_{M}~U_{F18}~A_{L}A_{F}~G_{M}A_{L}~C_{M}U_{F}~U_{M18}G_{F}~A_{M}G_{L}~A_{M}U_{F}~G_{M}~A_{F}~U_{L}~C_{F4}~C_{M}~A_{M}~A_{M}
P-U_{M}~U_{F18}~A_{L}A_{F}~G_{M}A_{L}~C_{M4}U_{F18}~U_{M}G_{F}~A_{M}G_{L}~A_{M}U_{F18}~G_{M}~A_{F}~U_{L18}~C_{F}~C_{M4}~A_{M}~A_{M}
P-U_{M}~U_{F18}~A_{L}A_{F}~G_{M}A_{L}~C_{M4}U_{F}~U_{M18}G_{F}~A_{M}G_{L}~A_{M}U_{F18}~G_{M}~A_{F}~U_{L}~C_{F4}~C_{M}~A_{M}~A_{M}
P-U_{M}~U_{F18}~A_{L}A_{F}~G_{M}A_{L}~C_{M4}U_{F18}~U_{M18}G_{F}~A_{M}G_{L}~A_{M}U_{F18}~G_{M}~A_{F}~U_{L}~C_{F4}~C_{M4}~A_{M}~A_{M}
P-U_{M}~U_{F}~A_{L}A_{F}~G_{M}A_{L}~C_{M4}U_{F18}~U_{M}G_{F}~A_{M}G_{L}~A_{M}U~G_{M}~A_{F}~U_{L18}~C_{F}~C_{M4}~A_{M}~A_{M}
P-U_{M}~U_{F}~A_{L}A_{F}~G_{M}A_{L}~C_{M4}U_{F18}~U_{M}G_{F}~A_{M}G_{L}~A_{M}U~G_{M}~A_{F}~U_{L18}~C_{F4}~C_{M}~A_{M}~A_{M}
P-U_{M}~U_{F}~A_{L}A_{F}~G_{M}A_{L}~C_{M}U_{F18}~U_{M18}G_{F}~A_{M}G_{L}~A_{M}U~G_{M}~A_{F}~U_{L}~C_{F4}~C_{M}~A_{M}~A_{M}
P-U_{M}~U_{F}~A_{L}A_{F}~G_{M}A_{L}~C_{M}U_{F18}~U_{M18}G_{F}~A_{M}G_{L}~A_{M}U~G_{M}~A_{F}~U_{L18}~C_{F}~C_{M4}~A_{M}~A_{M}
P-U_{M}~U_{F18}~A_{L}A_{F}~G_{M}A_{L}~C_{M}U_{F}~U_{M18}G_{F}~A_{M}G_{L}~A_{M}U~G_{M}~A_{F}~U_{L}~C_{F4}~C_{M}~A_{M}~A_{M}
P-U_{M}~U_{F18}~A_{L}A_{F}~G_{M}A_{L}~C_{M4}U_{F18}~U_{M}G_{F}~A_{M}G_{L}~A_{M}U~G_{M}~A_{F}~U_{L18}~C_{F}~C_{M4}~A_{M}~A_{M}
P-U_{M}~U_{F18}~A_{L}A_{F}~G_{M}A_{L}~C_{M4}U_{F}~U_{M18}G_{F}~A_{M}G_{L}~A_{M}U~G_{M}~A_{F}~U_{L}~C_{F4}~C_{M}~A_{M}~A_{M}
P-U_{M}~U_{F18}~A_{L}A_{F}~G_{M}A_{L}~C_{M4}U_{F18}~U_{M18}G_{F}~A_{M}G_{L}~A_{M}U~G_{M}~A_{F}~U_{L}~C_{F4}~C_{M4}~A_{M}~A_{M}
P-U_{M}~U_{F}~A_{L}A_{F}~G_{M}A_{L}~C_{M}U_{F}~U_{M}G_{F}~A_{M}G_{F}~A_{L}U_{F}~G_{M}~A_{L}~U_{M}~C_{F}~C_{L}~A_{M}~A_{M}
P-U_{M}~U_{F}~A_{L}A_{F}~G_{M}A_{L}~C_{M}U_{F}~U_{M}G_{F}~A_{M}G_{F}~A_{L}U~G_{M}~A_{L}~U_{M}~C_{F}~C_{L}~A_{M}~A_{M}
P-U_{M}~U_{F}~A_{L}A_{F}~G_{M}A_{L}~C_{M4}U_{F3}~U_{M}G_{F}~A_{M}G_{F}~A_{L}U_{F3}~G_{M}~A_{L}~U_{M3}~C_{F}~C_{L4}~A_{M}~A_{M}
P-U_{M}~U_{F}~A_{L}A_{F}~G_{M}A_{L}~C_{M4}U_{F3}~U_{M}G_{F}~A_{M}G_{F}~A_{L}U_{F3}~G_{M}~A_{L}~U_{M3}~C_{F4}~C_{L}~A_{M}~A_{M}
P-U_{M}~U_{F}~A_{L}A_{F}~G_{M}A_{L}~C_{M}U_{F3}~U_{M3}G_{F}~A_{M}G_{F}~A_{L}U_{F3}~G_{M}~A_{L}~U_{M}~C_{F4}~C_{L}~A_{M}~A_{M}
P-U_{M}~U_{F}~A_{L}A_{F}~G_{M}A_{L}~C_{M}U_{F3}~U_{M3}G_{F}~A_{M}G_{F}~A_{L}U_{F}~G_{M}~A_{L}~U_{M3}~C_{F}~C_{L4}~A_{M}~A_{M}
P-U_{M}~U_{F3}~A_{L}A_{F}~G_{M}A_{L}~C_{M}U_{F}~U_{M3}G_{F}~A_{M}G_{F}~A_{L}U_{F}~G_{M}~A_{L}~U_{M}~C_{F4}~C_{L}~A_{M}~A_{M}
P-U_{M}~U_{F3}~A_{L}A_{F}~G_{M}A_{L}~C_{M4}U_{F3}~U_{M}G_{F}~A_{M}G_{F}~A_{L}U_{F3}~G_{M}~A_{L}~U_{M3}~C_{F}~C_{L4}~A_{M}~A_{M}
P-U_{M}~U_{F3}~A_{L}A_{F}~G_{M}A_{L}~C_{M4}U_{F}~U_{M3}G_{F}~A_{M}G_{F}~A_{L}U_{F3}~G_{M}~A_{L}~U_{M}~C_{F4}~C_{L}~A_{M}~A_{M}
P-U_{M}~U_{F3}~A_{L}A_{F}~G_{M}A_{L}~C_{M4}U_{F3}~U_{M3}G_{F}~A_{M}G_{F}~A_{L}U_{F3}~G_{M}~A_{L}~U_{M}~C_{F4}~C_{L4}~A_{M}~A_{M}
P-U_{M}~U_{F}~A_{L}A_{F}~G_{M}A_{L}~C_{M4}U_{F3}~U_{M}G_{F}~A_{M}G_{F}~A_{L}U~G_{M}~A_{L}~U_{M3}~C_{F}~C_{L4}~A_{M}~A_{M}
P-U_{M}~U_{F}~A_{L}A_{F}~G_{M}A_{L}~C_{M4}U_{F3}~U_{M}G_{F}~A_{M}G_{F}~A_{L}U~G_{M}~A_{L}~U_{M3}~C_{F4}~C_{L}~A_{M}~A_{M}
P-U_{M}~U_{F}~A_{L}A_{F}~G_{M}A_{L}~C_{M}U_{F3}~U_{M3}G_{F}~A_{M}G_{F}~A_{L}U~G_{M}~A_{L}~U_{M}~C_{F4}~C_{L}~A_{M}~A_{M}
P-U_{M}~U_{F}~A_{L}A_{F}~G_{M}A_{L}~C_{M}U_{F3}~U_{M3}G_{F}~A_{M}G_{F}~A_{L}U~G_{M}~A_{L}~U_{M3}~C_{F}~C_{L4}~A_{M}~A_{M}
P-U_{M}~U_{F3}~A_{L}A_{F}~G_{M}A_{L}~C_{M}U_{F}~U_{M3}G_{F}~A_{M}G_{F}~A_{L}U~G_{M}~A_{L}~U_{M}~C_{F4}~C_{L}~A_{M}~A_{M}
P-U_{M}~U_{F3}~A_{L}A_{F}~G_{M}A_{L}~C_{M4}U_{F3}~U_{M}G_{F}~A_{M}G_{F}~A_{L}U~G_{M}~A_{L}~U_{M3}~C_{F}~C_{L4}~A_{M}~A_{M}
P-U_{M}~U_{F3}~A_{L}A_{F}~G_{M}A_{L}~C_{M4}U_{F}~U_{M3}G_{F}~A_{M}G_{F}~A_{L}U~G_{M}~A_{L}~U_{M}~C_{F4}~C_{L}~A_{M}~A_{M}
P-U_{M}~U_{F3}~A_{L}A_{F}~G_{M}A_{L}~C_{M4}U_{F3}~U_{M3}G_{F}~A_{M}G_{F}~A_{L}U~G_{M}~A_{L}~U_{M}~C_{F4}~C_{L4}~A_{M}~A_{M}
P-U_{M}~U_{F}~A_{L}A_{F}~G_{M}A_{L}~C_{M4}U_{F12}~U_{M}G_{F}~A_{M}G_{F}~A_{L}U_{F12}~G_{M}~A_{L}~U_{M12}~C_{F}~C_{L4}~A_{M}~A_{M}
P-U_{M}~U_{F}~A_{L}A_{F}~G_{M}A_{L}~C_{M4}U_{F12}~U_{M}G_{F}~A_{M}G_{F}~A_{L}U_{F12}~G_{M}~A_{L}~U_{M12}~C_{F4}~C_{L}~A_{M}~A_{M}
P-U_{M}~U_{F}~A_{L}A_{F}~G_{M}A_{L}~C_{M}U_{F12}~U_{M12}G_{F}~A_{M}G_{F}~A_{L}U_{F12}~G_{M}~A_{L}~U_{M}~C_{F4}~C_{L}~A_{M}~A_{M}
P-U_{M}~U_{F}~A_{L}A_{F}~G_{M}A_{L}~C_{M}U_{F12}~U_{M12}G_{F}~A_{M}G_{F}~A_{L}U_{F}~G_{M}~A_{L}~U_{M12}~C_{F}~C_{L4}~A_{M}~A_{M}
P-U_{M}~U_{F12}~A_{L}A_{F}~G_{M}A_{L}~C_{M}U_{F}~U_{M12}G_{F}~A_{M}G_{F}~A_{L}U_{F}~G_{M}~A_{L}~U_{M}~C_{F4}~C_{L}~A_{M}~A_{M}
P-U_{M}~U_{F12}~A_{L}A_{F}~G_{M}A_{L}~C_{M4}U_{F12}~U_{M}G_{F}~A_{M}G_{F}~A_{L}U_{F12}~G_{M}~A_{L}~U_{M12}~C_{F}~C_{L4}~A_{M}~A_{M}
P-U_{M}~U_{F12}~A_{L}A_{F}~G_{M}A_{L}~C_{M4}U_{F}~U_{M12}G_{F}~A_{M}G_{F}~A_{L}U_{F12}~G_{M}~A_{L}~U_{M}~C_{F4}~C_{L}~A_{M}~A_{M}
P-U_{M}~U_{F12}~A_{L}A_{F}~G_{M}A_{L}~C_{M4}U_{F12}~U_{M12}G_{F}~A_{M}G_{F}~A_{L}U_{F12}~G_{M}~A_{L}~U_{M}~C_{F4}~C_{L4}~A_{M}~A_{M}
P-U_{M}~U_{F}~A_{L}A_{F}~G_{M}A_{L}~C_{M4}U_{F12}~U_{M}G_{F}~A_{M}G_{F}~A_{L}U~G_{M}~A_{L}~U_{M12}~C_{F}~C_{L4}~A_{M}~A_{M}
P-U_{M}~U_{F}~A_{L}A_{F}~G_{M}A_{L}~C_{M4}U_{F12}~U_{M}G_{F}~A_{M}G_{F}~A_{L}U~G_{M}~A_{L}~U_{M12}~C_{F4}~C_{L}~A_{M}~A_{M}
P-U_{M}~U_{F}~A_{L}A_{F}~G_{M}A_{L}~C_{M}U_{F12}~U_{M12}G_{F}~A_{M}G_{F}~A_{L}U~G_{M}~A_{L}~U_{M}~C_{F4}~C_{L}~A_{M}~A_{M}
P-U_{M}~U_{F}~A_{L}A_{F}~G_{M}A_{L}~C_{M}U_{F12}~U_{M12}G_{F}~A_{M}G_{F}~A_{L}U~G_{M}~A_{L}~U_{M12}~C_{F}~C_{L4}~A_{M}~A_{M}
P-U_{M}~U_{F12}~A_{L}A_{F}~G_{M}A_{L}~C_{M}U_{F}~U_{M12}G_{F}~A_{M}G_{F}~A_{L}U~G_{M}~A_{L}~U_{M}~C_{F4}~C_{L}~A_{M}~A_{M}
P-U_{M}~U_{F12}~A_{L}A_{F}~G_{M}A_{L}~C_{M4}U_{F12}~U_{M}G_{F}~A_{M}G_{F}~A_{L}U~G_{M}~A_{L}~U_{M12}~C_{F}~C_{L4}~A_{M}~A_{M}
P-U_{M}~U_{F12}~A_{L}A_{F}~G_{M}A_{L}~C_{M4}U_{F}~U_{M12}G_{F}~A_{M}G_{F}~A_{L}U~G_{M}~A_{L}~U_{M}~C_{F4}~C_{L}~A_{M}~A_{M}
P-U_{M}~U_{F12}~A_{L}A_{F}~G_{M}A_{L}~C_{M4}U_{F12}~U_{M12}G_{F}~A_{M}G_{F}~A_{L}U~G_{M}~A_{L}~U_{M}~C_{F4}~C_{L4}~A_{M}~A_{M}
P-U_{M}~U_{F}~A_{L}A_{F}~G_{M}A_{L}~C_{M4}U_{F18}~U_{M}G_{F}~A_{M}G_{F}~A_{L}U_{F18}~G_{M}~A_{L}~U_{M18}~C_{F}~C_{L4}~A_{M}~A_{M}
P-U_{M}~U_{F}~A_{L}A_{F}~G_{M}A_{L}~C_{M4}U_{F18}~U_{M}G_{F}~A_{M}G_{F}~A_{L}U_{F18}~G_{M}~A_{L}~U_{M18}~C_{F4}~C_{L}~A_{M}~A_{M}
P-U_{M}~U_{F}~A_{L}A_{F}~G_{M}A_{L}~C_{M}U_{F18}~U_{M18}G_{F}~A_{M}G_{F}~A_{L}U_{F18}~G_{M}~A_{L}~U_{M}~C_{F4}~C_{L}~A_{M}~A_{M}
P-U_{M}~U_{F}~A_{L}A_{F}~G_{M}A_{L}~C_{M}U_{F18}~U_{M18}G_{F}~A_{M}G_{F}~A_{L}U_{F}~G_{M}~A_{L}~U_{M18}~C_{F}~C_{L4}~A_{M}~A_{M}
P-U_{M}~U_{F18}~A_{L}A_{F}~G_{M}A_{L}~C_{M}U_{F}~U_{M18}G_{F}~A_{M}G_{F}~A_{L}U_{F}~G_{M}~A_{L}~U_{M}~C_{F4}~C_{L}~A_{M}~A_{M}
P-U_{M}~U_{F18}~A_{L}A_{F}~G_{M}A_{L}~C_{M4}U_{F18}~U_{M}G_{F}~A_{M}G_{F}~A_{L}U_{F18}~G_{M}~A_{L}~U_{M18}~C_{F}~C_{L4}~A_{M}~A_{M}
P-U_{M}~U_{F18}~A_{L}A_{F}~G_{M}A_{L}~C_{M4}U_{F}~U_{M18}G_{F}~A_{M}G_{F}~A_{L}U_{F18}~G_{M}~A_{L}~U_{M}~C_{F4}~C_{L}~A_{M}~A_{M}
P-U_{M}~U_{F18}~A_{L}A_{F}~G_{M}A_{L}~C_{M4}U_{F18}~U_{M18}G_{F}~A_{M}G_{F}~A_{L}U_{F18}~G_{M}~A_{L}~U_{M}~C_{F4}~C_{L4}~A_{M}~A_{M}
P-U_{M}~U_{F}~A_{L}A_{F}~G_{M}A_{L}~C_{M4}U_{F18}~U_{M}G_{F}~A_{M}G_{F}~A_{L}U~G_{M}~A_{L}~U_{M18}~C_{F}~C_{L4}~A_{M}~A_{M}
P-U_{M}~U_{F}~A_{L}A_{F}~G_{M}A_{L}~C_{M4}U_{F18}~U_{M}G_{F}~A_{M}G_{F}~A_{L}U~G_{M}~A_{L}~U_{M18}~C_{F4}~C_{L}~A_{M}~A_{M}
P-U_{M}~U_{F}~A_{L}A_{F}~G_{M}A_{L}~C_{M}U_{F18}~U_{M18}G_{F}~A_{M}G_{F}~A_{L}U~G_{M}~A_{L}~U_{M}~C_{F4}~C_{L}~A_{M}~A_{M}
P-U_{M}~U_{F}~A_{L}A_{F}~G_{M}A_{L}~C_{M}U_{F18}~U_{M18}G_{F}~A_{M}G_{F}~A_{L}U~G_{M}~A_{L}~U_{M18}~C_{F}~C_{L4}~A_{M}~A_{M}
P-U_{M}~U_{F18}~A_{L}A_{F}~G_{M}A_{L}~C_{M}U_{F}~U_{M18}G_{F}~A_{M}G_{F}~A_{L}U~G_{M}~A_{L}~U_{M}~C_{F4}~C_{L}~A_{M}~A_{M}
P-U_{M}~U_{F18}~A_{L}A_{F}~G_{M}A_{L}~C_{M4}U_{F18}~U_{M}G_{F}~A_{M}G_{F}~A_{L}U~G_{M}~A_{L}~U_{M18}~C_{F}~C_{L4}~A_{M}~A_{M}
P-U_{M}~U_{F18}~A_{L}A_{F}~G_{M}A_{L}~C_{M4}U_{F}~U_{M18}G_{F}~A_{M}G_{F}~A_{L}U~G_{M}~A_{L}~U_{M}~C_{F4}~C_{L}~A_{M}~A_{M}
P-U_{M}~U_{F18}~A_{L}A_{F}~G_{M}A_{L}~C_{M4}U_{F18}~U_{M18}G_{F}~A_{M}G_{F}~A_{L}U~G_{M}~A_{L}~U_{M}~C_{F4}~C_{L4}~A_{M}~A_{M}
P-U_{M}~U_{F}~A_{M}A_{L}~G_{M}A_{L}~C_{M}U_{F}~U_{M}G_{F}~A_{L}G_{F}~A_{L}U_{F}~G_{L}~A_{F}~U_{L}~C_{F}~C_{M}~A_{M}~A_{M}
P-U_{M}~U_{F}~A_{M}A_{L}~G_{M}A_{L}~C_{M}U_{F}~U_{M}G_{F}~A_{L}G_{F}~A_{L}U~G_{L}~A_{F}~U_{L}~C_{F}~C_{M}~A_{M}~A_{M}
P-U_{M}~U_{F}~A_{M}A_{L}~G_{M}A_{L}~C_{M4}U_{F3}~U_{M}G_{F}~A_{L}G_{F}~A_{L}U_{F3}~G_{L}~A_{F}~U_{L3}~C_{F}~C_{M4}~A_{M}~A_{M}
P-U_{M}~U_{F}~A_{M}A_{L}~G_{M}A_{L}~C_{M4}U_{F3}~U_{M}G_{F}~A_{L}G_{F}~A_{L}U_{F3}~G_{L}~A_{F}~U_{L3}~C_{F4}~C_{M}~A_{M}~A_{M}
P-U_{M}~U_{F}~A_{M}A_{L}~G_{M}A_{L}~C_{M}U_{F3}~U_{M3}G_{F}~A_{L}G_{F}~A_{L}U_{F3}~G_{L}~A_{F}~U_{L}~C_{F4}~C_{M}~A_{M}~A_{M}
P-U_{M}~U_{F}~A_{M}A_{L}~G_{M}A_{L}~C_{M}U_{F3}~U_{M3}G_{F}~A_{L}G_{F}~A_{L}U_{F}~G_{L}~A_{F}~U_{L3}~C_{F}~C_{M4}~A_{M}~A_{M}
P-U_{M}~U_{F3}~A_{M}A_{L}~G_{M}A_{L}~C_{M}U_{F}~U_{M3}G_{F}~A_{L}G_{F}~A_{L}U_{F}~G_{L}~A_{F}~U_{L}~C_{F4}~C_{M}~A_{M}~A_{M}
P-U_{M}~U_{F3}~A_{M}A_{L}~G_{M}A_{L}~C_{M4}U_{F3}~U_{M}G_{F}~A_{L}G_{F}~A_{L}U_{F3}~G_{L}~A_{F}~U_{L3}~C_{F}~C_{M4}~A_{M}~A_{M}
P-U_{M}~U_{F3}~A_{M}A_{L}~G_{M}A_{L}~C_{M4}U_{F}~U_{M3}G_{F}~A_{L}G_{F}~A_{L}U_{F3}~G_{L}~A_{F}~U_{L}~C_{F4}~C_{M}~A_{M}~A_{M}
P-U_{M}~U_{F3}~A_{M}A_{L}~G_{M}A_{L}~C_{M4}U_{F3}~U_{M3}G_{F}~A_{L}G_{F}~A_{L}U_{F3}~G_{L}~A_{F}~U_{L}~C_{F4}~C_{M4}~A_{M}~A_{M}
P-U_{M}~U_{F}~A_{M}A_{L}~G_{M}A_{L}~C_{M4}U_{F3}~U_{M}G_{F}~A_{L}G_{F}~A_{L}U~G_{L}~A_{F}~U_{L3}~C_{F}~C_{M4}~A_{M}~A_{M}
P-U_{M}~U_{F}~A_{M}A_{L}~G_{M}A_{L}~C_{M4}U_{F3}~U_{M}G_{F}~A_{L}G_{F}~A_{L}U~G_{L}~A_{F}~U_{L3}~C_{F4}~C_{M}~A_{M}~A_{M}
P-U_{M}~U_{F}~A_{M}A_{L}~G_{M}A_{L}~C_{M}U_{F3}~U_{M3}G_{F}~A_{L}G_{F}~A_{L}U~G_{L}~A_{F}~U_{L}~C_{F4}~C_{M}~A_{M}~A_{M}
P-U_{M}~U_{F}~A_{M}A_{L}~G_{M}A_{L}~C_{M}U_{F3}~U_{M3}G_{F}~A_{L}G_{F}~A_{L}U~G_{L}~A_{F}~U_{L3}~C_{F}~C_{M4}~A_{M}~A_{M}
P-U_{M}~U_{F3}~A_{M}A_{L}~G_{M}A_{L}~C_{M}U_{F}~U_{M3}G_{F}~A_{L}G_{F}~A_{L}U~G_{L}~A_{F}~U_{L}~C_{F4}~C_{M}~A_{M}~A_{M}
P-U_{M}~U_{F3}~A_{M}A_{L}~G_{M}A_{L}~C_{M4}U_{F3}~U_{M}G_{F}~A_{L}G_{F}~A_{L}U~G_{L}~A_{F}~U_{L3}~C_{F}~C_{M4}~A_{M}~A_{M}
P-U_{M}~U_{F3}~A_{M}A_{L}~G_{M}A_{L}~C_{M4}U_{F}~U_{M3}G_{F}~A_{L}G_{F}~A_{L}U~G_{L}~A_{F}~U_{L}~C_{F4}~C_{M}~A_{M}~A_{M}
P-U_{M}~U_{F3}~A_{M}A_{L}~G_{M}A_{L}~C_{M4}U_{F3}~U_{M3}G_{F}~A_{L}G_{F}~A_{L}U~G_{L}~A_{F}~U_{L}~C_{F4}~C_{M4}~A_{M}~A_{M}
P-U_{M}~U_{F}~A_{M}A_{L}~G_{M}A_{L}~C_{M4}U_{F12}~U_{M}G_{F}~A_{L}G_{F}~A_{L}U_{F12}~G_{L}~A_{F}~U_{L12}~C_{F}~C_{M4}~A_{M}~A_{M}
P-U_{M}~U_{F}~A_{M}A_{L}~G_{M}A_{L}~C_{M4}U_{F12}~U_{M}G_{F}~A_{L}G_{F}~A_{L}U_{F12}~G_{L}~A_{F}~U_{L12}~C_{F4}~C_{M}~A_{M}~A_{M}
P-U_{M}~U_{F}~A_{M}A_{L}~G_{M}A_{L}~C_{M}U_{F12}~U_{M12}G_{F}~A_{L}G_{F}~A_{L}U_{F12}~G_{L}~A_{F}~U_{L}~C_{F4}~C_{M}~A_{M}~A_{M}
P-U_{M}~U_{F}~A_{M}A_{L}~G_{M}A_{L}~C_{M}U_{F12}~U_{M12}G_{F}~A_{L}G_{F}~A_{L}U_{F}~G_{L}~A_{F}~U_{L12}~C_{F}~C_{M4}~A_{M}~A_{M}
P-U_{M}~U_{F12}~A_{M}A_{L}~G_{M}A_{L}~C_{M}U_{F}~U_{M12}G_{F}~A_{L}G_{F}~A_{L}U_{F}~G_{L}~A_{F}~U_{L}~C_{F4}~C_{M}~A_{M}~A_{M}
P-U_{M}~U_{F12}~A_{M}A_{L}~G_{M}A_{L}~C_{M4}U_{F12}~U_{M}G_{F}~A_{L}G_{F}~A_{L}U_{F12}~G_{L}~A_{F}~U_{L12}~C_{F}~C_{M4}~A_{M}~A_{M}
P-U_{M}~U_{F12}~A_{M}A_{L}~G_{M}A_{L}~C_{M4}U_{F}~U_{M12}G_{F}~A_{L}G_{F}~A_{L}U_{F12}~G_{L}~A_{F}~U_{L}~C_{F4}~C_{M}~A_{M}~A_{M}
P-U_{M}~U_{F12}~A_{M}A_{L}~G_{M}A_{L}~C_{M4}U_{F12}~U_{M12}G_{F}~A_{L}G_{F}~A_{L}U_{F12}~G_{L}~A_{F}~U_{L}~C_{F4}~C_{M4}~A_{M}~A_{M}
P-U_{M}~U_{F}~A_{M}A_{L}~G_{M}A_{L}~C_{M4}U_{F12}~U_{M}G_{F}~A_{L}G_{F}~A_{L}U~G_{L}~A_{F}~U_{L12}~C_{F}~C_{M4}~A_{M}~A_{M}
P-U_{M}~U_{F}~A_{M}A_{L}~G_{M}A_{L}~C_{M4}U_{F12}~U_{M}G_{F}~A_{L}G_{F}~A_{L}U~G_{L}~A_{F}~U_{L12}~C_{F4}~C_{M}~A_{M}~A_{M}
P-U_{M}~U_{F}~A_{M}A_{L}~G_{M}A_{L}~C_{M}U_{F12}~U_{M12}G_{F}~A_{L}G_{F}~A_{L}U~G_{L}~A_{F}~U_{L}~C_{F4}~C_{M}~A_{M}~A_{M}
P-U_{M}~U_{F}~A_{M}A_{L}~G_{M}A_{L}~C_{M}U_{F12}~U_{M12}G_{F}~A_{L}G_{F}~A_{L}U~G_{L}~A_{F}~U_{L12}~C_{F}~C_{M4}~A_{M}~A_{M}
P-U_{M}~U_{F12}~A_{M}A_{L}~G_{M}A_{L}~C_{M}U_{F}~U_{M12}G_{F}~A_{L}G_{F}~A_{L}U~G_{L}~A_{F}~U_{L}~C_{F4}~C_{M}~A_{M}~A_{M}
P-U_{M}~U_{F12}~A_{M}A_{L}~G_{M}A_{L}~C_{M4}U_{F12}~U_{M}G_{F}~A_{L}G_{F}~A_{L}U~G_{L}~A_{F}~U_{L12}~C_{F}~C_{M4}~A_{M}~A_{M}
P-U_{M}~U_{F12}~A_{M}A_{L}~G_{M}A_{L}~C_{M4}U_{F}~U_{M12}G_{F}~A_{L}G_{F}~A_{L}U~G_{L}~A_{F}~U_{L}~C_{F4}~C_{M}~A_{M}~A_{M}
P-U_{M}~U_{F12}~A_{M}A_{L}~G_{M}A_{L}~C_{M4}U_{F12}~U_{M12}G_{F}~A_{L}G_{F}~A_{L}U~G_{L}~A_{F}~U_{L}~C_{F4}~C_{M4}~A_{M}~A_{M}
P-U_{M}~U_{F}~A_{M}A_{L}~G_{M}A_{L}~C_{M4}U_{F18}~U_{M}G_{F}~A_{L}G_{F}~A_{L}U_{F18}~G_{L}~A_{F}~U_{L18}~C_{F}~C_{M4}~A_{M}~A_{M}
P-U_{M}~U_{F}~A_{M}A_{L}~G_{M}A_{L}~C_{M4}U_{F18}~U_{M}G_{F}~A_{L}G_{F}~A_{L}U_{F18}~G_{L}~A_{F}~U_{L18}~C_{F4}~C_{M}~A_{M}~A_{M}
P-U_{M}~U_{F}~A_{M}A_{L}~G_{M}A_{L}~C_{M}U_{F18}~U_{M18}G_{F}~A_{L}G_{F}~A_{L}U_{F18}~G_{L}~A_{F}~U_{L}~C_{F4}~C_{M}~A_{M}~A_{M}
P-U_{M}~U_{F}~A_{M}A_{L}~G_{M}A_{L}~C_{M}U_{F18}~U_{M18}G_{F}~A_{L}G_{F}~A_{L}U_{F}~G_{L}~A_{F}~U_{L18}~C_{F}~C_{M4}~A_{M}~A_{M}
P-U_{M}~U_{F18}~A_{M}A_{L}~G_{M}A_{L}~C_{M}U_{F}~U_{M18}G_{F}~A_{L}G_{F}~A_{L}U_{F}~G_{L}~A_{F}~U_{L}~C_{F4}~C_{M}~A_{M}~A_{M}
P-U_{M}~U_{F18}~A_{M}A_{L}~G_{M}A_{L}~C_{M4}U_{F18}~U_{M}G_{F}~A_{L}G_{F}~A_{L}U_{F18}~G_{L}~A_{F}~U_{L18}~C_{F}~C_{M4}~A_{M}~A_{M}
P-U_{M}~U_{F18}~A_{M}A_{L}~G_{M}A_{L}~C_{M4}U_{F}~U_{M18}G_{F}~A_{L}G_{F}~A_{L}U_{F18}~G_{L}~A_{F}~U_{L}~C_{F4}~C_{M}~A_{M}~A_{M}
P-U_{M}~U_{F18}~A_{M}A_{L}~G_{M}A_{L}~C_{M4}U_{F18}~U_{M18}G_{F}~A_{L}G_{F}~A_{L}U_{F18}~G_{L}~A_{F}~U_{L}~C_{F4}~C_{M4}~A_{M}~A_{M}
P-U_{M}~U_{F}~A_{M}A_{L}~G_{M}A_{L}~C_{M4}U_{F18}~U_{M}G_{F}~A_{L}G_{F}~A_{L}U~G_{L}~A_{F}~U_{L18}~C_{F}~C_{M4}~A_{M}~A_{M}
P-U_{M}~U_{F}~A_{M}A_{L}~G_{M}A_{L}~C_{M4}U_{F18}~U_{M}G_{F}~A_{L}G_{F}~A_{L}U~G_{L}~A_{F}~U_{L18}~C_{F4}~C_{M}~A_{M}~A_{M}
P-U_{M}~U_{F}~A_{M}A_{L}~G_{M}A_{L}~C_{M}U_{F18}~U_{M18}G_{F}~A_{L}G_{F}~A_{L}U~G_{L}~A_{F}~U_{L}~C_{F4}~C_{M}~A_{M}~A_{M}
P-U_{M}~U_{F}~A_{M}A_{L}~G_{M}A_{L}~C_{M}U_{F18}~U_{M18}G_{F}~A_{L}G_{F}~A_{L}U~G_{L}~A_{F}~U_{L18}~C_{F}~C_{M4}~A_{M}~A_{M}
P-U_{M}~U_{F18}~A_{M}A_{L}~G_{M}A_{L}~C_{M}U_{F}~U_{M18}G_{F}~A_{L}G_{F}~A_{L}U~G_{L}~A_{F}~U_{L}~C_{F4}~C_{M}~A_{M}~A_{M}
P-U_{M}~U_{F18}~A_{M}A_{L}~G_{M}A_{L}~C_{M4}U_{F18}~U_{M}G_{F}~A_{L}G_{F}~A_{L}U~G_{L}~A_{F}~U_{L18}~C_{F}~C_{M4}~A_{M}~A_{M}
P-U_{M}~U_{F18}~A_{M}A_{L}~G_{M}A_{L}~C_{M4}U_{F}~U_{M18}G_{F}~A_{L}G_{F}~A_{L}U~G_{L}~A_{F}~U_{L}~C_{F4}~C_{M}~A_{M}~A_{M}
P-U_{M}~U_{F18}~A_{M}A_{L}~G_{M}A_{L}~C_{M4}U_{F18}~U_{M18}G_{F}~A_{L}G_{F}~A_{L}U~G_{L}~A_{F}~U_{L}~C_{F4}~C_{M4}~A_{M}~A_{M}

### Table 12

### Examples of Murine APO-CIII ags-siRNA Compositions

### A. Murine Apo-CIII Antisense Sequence:

UAUGAGAACAUACUUUCCC

Lima et al. (Cell 150: 883-94, 2012) used this sequence to generate ssRNAi compounds against human/murine PTEN including the following:

5′-VP~Tₘₒₑ~A_{F}~T_{M}G_{F}~A_{M}G_{F}~A_{M}A_{F}~C_{M}A_{F}~T_{M}A_{F}~C_{M}T_{F}~T_{M}~T_{F}~C_{M}~C_{F}~C_{M}~Aₘₒₑ~Aₘₒₑ

### With the 5′-VP, T and 2′-MOE Modifications Removed and Replaced by P, U and 2′-O-methyl:

P-U_{M}~A_{F}~U_{M}G_{F}~A_{M}G_{F}~A_{M}A_{F}~C_{M}A_{F}~U_{M}A_{F}~C_{M}U_{F}~U_{M}~U_{F}~C_{M}~C_{F}~C_{M}~A_{M}~A_{M}

Any of the 5′-end nucleosides (P-U_{M}) shown in the examples of agsRNAi compounds in Table 12 can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-A_{F}, P-A_{M}, P-A_{N}, P-A_{L}, P-A_{Z}, P-A_{H}, P-A_{B}, P-A_{K}, P-A_{Q}, P-A_{S}, P-A_{T}, P-A_{W}, P-A_{V}, P-A_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 12 with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the compounds in Table 12 can any of those units and linkages provided for herein for overhang precursors.

B. Examples of Application of AGSD to this Compound:

Compositions of Matter Suitable for Administration to a Subject
with or without a Protective Carrier and for Any Other use
as Provided for Herein (5′-3′)

P-U_{M}~A_{F}~U_{M}G_{F}~A_{M}G_{F}~A_{M}A_{F}~C_{M}A_{F}~U_{M}~A_{F}~C_{M}U_{F3}~U_{M}~U_{F3}~C_{M}~C_{F4}~C_{M}~A_{M}~A_{M}
P-U_{M}~A_{F}~U_{M}G_{F}~A_{M}G_{F}~A_{M}A_{F}~C_{M4}A_{F}~U_{M}~A_{F}~C_{M4}U_{F}~U_{M3}~U_{F}~C_{M4}~C_{F}~C_{M}~A_{M}~A_{M}
P-U_{M}~A_{F}~U_{M}G_{F}~A_{M}G_{F}~A_{M}A_{F}~C_{M4}A_{F}~U_{M3}A_{F}~C_{M4}U_{F3}~U_{M3}~U_{F3}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U_{F3}~U_{M}~U_{F3}~C_{M}~C_{F4}~C_{M}~A_{M}~A_{M}
P-U_{M}~A_{F}~U_{M3}G_{F}~A_{M}G_{F}~A_{M}A_{F}~C_{M4}A_{F}~U_{M}~A_{F}~C_{M4}U_{F}~U_{M3}~U_{F}~C_{M4}~C_{F}~C_{M4}~A_{M}~A_{M}
P-U_{M}~A_{F}~U_{M3}G_{F}~A_{M}G_{F}~A_{M}A_{F}~C_{M4}A_{F}~U_{M3}A_{F}~C_{M4}U_{F3}~U_{M3}~U_{F3}~C_{M4}~C_{F4}~C_{M4}~A_{M}~A_{M}
P-U_{M}~A_{F}~U_{M3}G_{F}~A_{M}G_{F}~A_{M}A_{F}~C_{M}A_{F}~U_{M}~A_{F}~C_{M}U_{F3}~U_{M}~U_{F3}~C_{M}~C_{F4}~C_{M}~A_{M}~A_{M}
P-U_{M}~A_{F}~U_{M3}G_{F}~A_{M}G_{F}~A_{M}A_{F}~C_{M4}A_{F}~U_{M}~A_{F}~C_{M4}U_{F}~U_{M3}~U_{F}~C_{M4}~C_{F}~C_{M4}~A_{M}~A_{M}
P-U_{M}~A_{F}~U_{M3}G_{F}~A_{M}G_{F}~A_{M}A_{F}~C_{M4}A_{F}~U_{M3}A_{F}~C_{M4}U_{F3}~U_{M3}~U_{F3}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U_{F12}~U_{M}~U_{F12}~C_{M}~C_{F4}~C_{M}~A_{M}~A_{M}
P-U_{M}~A_{F}~U_{M}G_{F}~A_{M}G_{F}~A_{M}A_{F}~C_{M4}A_{F}~U_{M}~A_{F}~C_{M4}U_{F}~U_{M12}~U_{F}~C_{M4}~C_{F}~C_{M4}~A_{M}~A_{M}
P-U_{M}~A_{F}~U_{M}G_{F}~A_{M}G_{F}~A_{M}A_{F}~C_{M4}A_{F}~U_{M12}A_{F}~C_{M4}U_{F12}~U_{M12}~U_{F12}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U_{F12}~U_{M}~U_{F12}~C_{M}~C_{F4}~C_{M}~A_{M}~A_{M}
P-U_{M}~A_{F}~U_{M12}G_{F}~A_{M}G_{F}~A_{M}A_{F}~C_{M4}A_{F}~U_{M}~A_{F}~C_{M4}U_{F}~U_{M12}~U_{F}~C_{M4}~C_{F}~C_{M4}~A_{M}~A_{M}
P-U_{M}~A_{F}~U_{M12}G_{F}~A_{M}G_{F}~A_{M}A_{F}~C_{M4}A_{F}~U_{M12}A_{F}~C_{M4}U_{F12}~U_{M12}~U_{F12}~C_{M4}~C_{F4}~C_{M4}~A_{M}~A_{M}
P-U_{M}~A_{F}~U_{M12}G_{F}~A_{M}G_{F}~A_{M}A_{F}~C_{M}A_{F}~U_{M}~A_{F}~C_{M}U_{F12}~U_{M}~U_{F12}~C_{M}~C_{F4}~C_{M}~A_{M}~A_{M}
P-U_{M}~A_{F}~U_{M12}G_{F}~A_{M}G_{F}~A_{M}A_{F}~C_{M4}A_{F}~U_{M}~A_{F}~C_{M4}U_{F}~U_{M12}~U_{F}~C_{M4}~C_{F}~C_{M4}~A_{M}~A_{M}
P-U_{M}~A_{F}~U_{M12}G_{F}~A_{M}G_{F}~A_{M}A_{F}~C_{M4}A_{F}~U_{M12}A_{F}~C_{M4}U_{F12}~U_{M12}~U_{F12}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U_{F18}~U_{M}~U_{F18}~C_{M}~C_{F4}~C_{M}~A_{M}~A_{M}
P-U_{M}~A_{F}~U_{M}G_{F}~A_{M}G_{F}~A_{M}A_{F}~C_{M4}A_{F}~U_{M}~A_{F}~C_{M4}U_{F}~U_{M18}~U_{F}~C_{M4}~C_{F}~C_{M4}~A_{M}~A_{M}
P-U_{M}~A_{F}~U_{M}G_{F}~A_{M}G_{F}~A_{M}A_{F}~C_{M4}A_{F}~U_{M18}A_{F}~C_{M4}U_{F18}~U_{M18}~U_{F18}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U_{F18}~U_{M}~U_{F18}~C_{M}~C_{F4}~C_{M}~A_{M}~A_{M}
P-U_{M}~A_{F}~U_{M18}G_{F}~A_{M}G_{F}~A_{M}A_{F}~C_{M4}A_{F}~U_{M}~A_{F}~C_{M4}U_{F}~U_{M18}~U_{F}~C_{M4}~C_{F}~C_{M4}~A_{M}~A_{M}
P-U_{M}~A_{F}~U_{M18}G_{F}~A_{M}G_{F}~A_{M}A_{F}~C_{M4}A_{F}~U_{M18}A_{F}~C_{M4}U_{F18}~U_{M18}~U_{F18}~C_{M4}~C_{F4}~C_{M4}~A_{M}~A_{M}
P-U_{M}~A_{F}~U_{M18}G_{F}~A_{M}G_{F}~A_{M}A_{F}~C_{M}A_{F}~U_{M}~A_{F}~C_{M}U_{F18}~U_{M}~U_{F18}~C_{M}~C_{F4}~C_{M}~A_{M}~A_{M}
P-U_{M}~A_{F}~U_{M18}G_{F}~A_{M}G_{F}~A_{M}A_{F}~C_{M4}A_{F}~U_{M}~A_{F}~C_{M4}U_{F}~U_{M18}~U_{F}~C_{M4}~C_{F}~C_{M4}~A_{M}~A_{M}
P-U_{M}~A_{F}~U_{M18}G_{F}~A_{M}G_{F}~A_{M}A_{F}~C_{M4}A_{F}~U_{M18}A_{F}~C_{M4}U_{F18}~U_{M18}~U_{F18}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U~U_{M}~U_{F3}~C_{M}~C_{F4}~C_{M}~A_{M}~A_{M}
P-U_{M}~A_{F}~U_{M}G_{F}~A_{M}G_{F}~A_{M}A_{F}~C_{M4}A_{F}~U_{M}~A_{F}~C_{M4}U~U_{M3}~U_{F}~C_{M4}~C_{F}~C_{M4}~A_{M}~A_{M}
P-U_{M}~A_{F}~U_{M}G_{F}~A_{M}G_{F}~A_{M}A_{F}~C_{M4}A_{F}~U_{M3}A_{F}~C_{M4}U~U_{M3}~U_{F3}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U~U_{M}~U_{F3}~C_{M}~C_{F4}~C_{M}~A_{M}~A_{M}
P-U_{M}~A_{F}~U_{M3}G_{F}~A_{M}G_{F}~A_{M}A_{F}~C_{M4}A_{F}~U_{M}~A_{F}~C_{M4}U~U_{M3}~U_{F}~C_{M4}~C_{F}~C_{M4}~A_{M}~A_{M}
P-U_{M}~A_{F}~U_{M3}G_{F}~A_{M}G_{F}~A_{M}A_{F}~C_{M4}A_{F}~U_{M3}A_{F}~C_{M4}U~U_{M3}~U_{F3}~C_{M4}~C_{F4}~C_{M4}~A_{M}~A_{M}
P-U_{M}~A_{F}~U_{M3}G_{F}~A_{M}G_{F}~A_{M}A_{F}~C_{M}A_{F}~U_{M}~A_{F}~C_{M}U~U_{M}~U_{F3}~C_{M}~C_{F4}~C_{M}~A_{M}~A_{M}
P-U_{M}~A_{F}~U_{M3}G_{F}~A_{M}G_{F}~A_{M}A_{F}~C_{M4}A_{F}~U_{M}~A_{F}~C_{M4}U~U_{M3}~U_{F}~C_{M4}~C_{F}~C_{M4}~A_{M}~A_{M}
P-U_{M}~A_{F}~U_{M3}G_{F}~A_{M}G_{F}~A_{M}A_{F}~C_{M4}A_{F}~U_{M3}A_{F}~C_{M4}U~U_{M3}~U_{F3}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U~U_{M}~U_{F12}~C_{M}~C_{F4}~C_{M}~A_{M}~A_{M}
P-U_{M}~A_{F}~U_{M}G_{F}~A_{M}G_{F}~A_{M}A_{F}~C_{M4}A_{F}~U_{M}~A_{F}~C_{M4}U~U_{M12}~U_{F}~C_{M4}~C_{F}~C_{M4}~A_{M}~A_{M}
P-U_{M}~A_{F}~U_{M}G_{F}~A_{M}G_{F}~A_{M}A_{F}~C_{M4}A_{F}~U_{M12}A_{F}~C_{M4}U~U_{M12}~U_{F12}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U~U_{M}~U_{F12}~C_{M}~C_{F4}~C_{M}~A_{M}~A_{M}
P-U_{M}~A_{F}~U_{M12}G_{F}~A_{M}G_{F}~A_{M}A_{F}~C_{M4}A_{F}~U_{M}~A_{F}~C_{M4}U~U_{M12}~U_{F}~C_{M4}~C_{F}~C_{M4}~A_{M}~A_{M}
P-U_{M}~A_{F}~U_{M12}G_{F}~A_{M}G_{F}~A_{M}A_{F}~C_{M4}A_{F}~U_{M12}A_{F}~C_{M4}U~U_{M12}~U_{F12}~C_{M4}~C_{F4}~C_{M4}~A_{M}~A_{M}
P-U_{M}~A_{F}~U_{M12}G_{F}~A_{M}G_{F}~A_{M}A_{F}~C_{M}A_{F}~U_{M}~A_{F}~C_{M}U~U_{M}~U_{F12}~C_{M}~C_{F4}~C_{M}~A_{M}~A_{M}
P-U_{M}~A_{F}~U_{M12}G_{F}~A_{M}G_{F}~A_{M}A_{F}~C_{M4}A_{F}~U_{M}~A_{F}~C_{M4}U~U_{M12}~U_{F}~C_{M4}~C_{F}~C_{M4}~A_{M}~A_{M}
P-U_{M}~A_{F}~U_{M12}G_{F}~A_{M}G_{F}~A_{M}A_{F}~C_{M4}A_{F}~U_{M12}A_{F}~C_{M4}U~U_{M12}~U_{F12}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U~U_{M}~U_{F18}~C_{M}~C_{F4}~C_{M}~A_{M}~A_{M}
P-U_{M}~A_{F}~U_{M}G_{F}~A_{M}G_{F}~A_{M}A_{F}~C_{M4}A_{F}~U_{M}~A_{F}~C_{M4}U~U_{M18}~U_{F}~C_{M4}~C_{F}~C_{M4}~A_{M}~A_{M}
P-U_{M}~A_{F}~U_{M}G_{F}~A_{M}G_{F}~A_{M}A_{F}~C_{M4}A_{F}~U_{M18}A_{F}~C_{M4}U~U_{M18}~U_{F18}~C_{M4}~C_{F4}~C_{M4}~A_{M}~A_{M}
P-U_{M}~A_{F}~U_{M}G_{F}~A_{M}G_{F}~A_{M}A_{F}~C_{M}A_{F}~U_{M}~A_{F}~C_{M}U~U_{M}~U_{F18}~C_{M}~C_{F4}~C_{M}~A_{M}~A_{M}
P-U_{M}~A_{F}~U_{M18}G_{F}~A_{M}G_{F}~A_{M}A_{F}~C_{M4}A_{F}~U_{M}~A_{F}~C_{M4}U~U_{M18}~U_{F}~C_{M4}~C_{F}~C_{M4}~A_{M}~A_{M}
P-U_{M}~A_{F}~U_{M18}G_{F}~A_{M}G_{F}~A_{M}A_{F}~C_{M4}A_{F}~U_{M18}A_{F}~C_{M4}U~U_{M18}~U_{F18}~C_{M4}~C_{F4}~C_{M4}~A_{M}~A_{M}
P-U_{M}~A_{F}~U_{M18}G_{F}~A_{M}G_{F}~A_{M}A_{F}~C_{M}A_{F}~U_{M}~A_{F}~C_{M}U~U_{M}~U_{F18}~C_{M}~C_{F4}~C_{M}~A_{M}~A_{M}
P-U_{M}~A_{F}~U_{M18}G_{F}~A_{M}G_{F}~A_{M}A_{F}~C_{M4}A_{F}~U_{M}~A_{F}~C_{M4}U~U_{M18}~U_{F}~C_{M4}~C_{F}~C_{M4}~A_{M}~A_{M}
P-U_{M}~A_{F}~U_{M18}G_{F}~A_{M}G_{F}~A_{M}A_{F}~C_{M4}A_{F}~U_{M18}A_{F}~C_{M4}U~U_{M18}~U_{F18}~C_{M4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U_{F}~U_{M}~U_{F}~C_{L}~C_{F}~C_{M}~A_{M}~A_{M}
P-U_{M}~A_{F}~U_{M}G_{L}~A_{M}G_{F}~A_{L}A_{F}~C_{M}A_{L}~U_{M}A_{L}~C_{M}U~U_{M}~U_{F}~C_{L}~C_{F}~C_{M}~A_{M}~A_{M}
P-U_{M}~A_{F}~U_{M}G_{L}~A_{M}G_{F}~A_{L}A_{F}~C_{M}A_{L}~U_{M}A_{L}~C_{M}U_{F3}~U_{M}~U_{F3}~C_{L}~C_{F4}~C_{M}~A_{M}~A_{M}
P-U_{M}~A_{F}~U_{M}G_{L}~A_{M}G_{F}~A_{L}A_{F}~C_{M4}A_{L}~U_{M}A_{L}~C_{M4}U_{F}~U_{M3}~U_{F}~C_{L4}~C_{F}~C_{M4}~A_{M}~A_{M}
P-U_{M}~A_{F}~U_{M}G_{L}~A_{M}G_{F}~A_{L}A_{F}~C_{M4}A_{L}~U_{M3}A_{L}~C_{M4}U_{F3}~U_{M3}~U_{F3}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U_{F3}~U_{M}~U_{F3}~C_{L}~C_{F4}~C_{M}~A_{M}~A_{M}
P-U_{M}~A_{F}~U_{M3}G_{L}~A_{M}G_{F}~A_{L}A_{F}~C_{M4}A_{L}~U_{M}A_{L}~C_{M4}U_{F}~U_{M3}~U_{F}~C_{L4}~C_{F}~C_{M4}~A_{M}~A_{M}
P-U_{M}~A_{F}~U_{M3}G_{L}~A_{M}G_{F}~A_{L}A_{F}~C_{M4}A_{L}~U_{M3}A_{L}~C_{M4}U_{F3}~U_{M3}~U_{F3}~C_{L4}~C_{F4}~C_{M4}~A_{M}~A_{M}
P-U_{M}~A_{F}~U_{M3}G_{L}~A_{M}G_{F}~A_{L}A_{F}~C_{M}A_{L}~U_{M}A_{L}~C_{M}U_{F3}~U_{M}~U_{F3}~C_{L}~C_{F4}~C_{M}~A_{M}~A_{M}
P-U_{M}~A_{F}~U_{M3}G_{L}~A_{M}G_{F}~A_{L}A_{F}~C_{M4}A_{L}~U_{M}A_{L}~C_{M4}U_{F}~U_{M3}~U_{F}~C_{L4}~C_{F}~C_{M4}~A_{M}~A_{M}
P-U_{M}~A_{F}~U_{M3}G_{L}~A_{M}G_{F}~A_{L}A_{F}~C_{M4}A_{L}~U_{M3}A_{L}~C_{M4}U_{F3}~U_{M3}~U_{F3}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U_{F12}~U_{M}~U_{F12}~C_{L}~C_{F4}~C_{M}~A_{M}~A_{M}
P-U_{M}~A_{F}~U_{M}G_{L}~A_{M}G_{F}~A_{L}A_{F}~C_{M4}A_{L}~U_{M}A_{L}~C_{M4}U_{F}~U_{M12}~U_{F}~C_{L4}~C_{F}~C_{M4}~A_{M}~A_{M}
P-U_{M}~A_{F}~U_{M}G_{L}~A_{M}G_{F}~A_{L}A_{F}~C_{M4}A_{L}~U_{M12}A_{L}~C_{M4}U_{F12}~U_{M12}~U_{F12}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U_{F12}~U_{M}~U_{F12}~C_{L}~C_{F4}~C_{M}~A_{M}~A_{M}
P-U_{M}~A_{F}~U_{M12}G_{L}~A_{M}G_{F}~A_{L}A_{F}~C_{M4}A_{L}~U_{M}A_{L}~C_{M4}U_{F}~U_{M12}~U_{F}~C_{L4}~C_{F}~C_{M4}~A_{M}~A_{M}
P-U_{M}~A_{F}~U_{M12}G_{L}~A_{M}G_{F}~A_{L}A_{F}~C_{M4}A_{L}~U_{M12}A_{L}~C_{M4}U_{F12}~U_{M12}~U_{F12}~C_{L4}~C_{F4}~C_{M4}~A_{M}~A_{M}
P-U_{M}~A_{F}~U_{M12}G_{L}~A_{M}G_{F}~A_{L}A_{F}~C_{M}A_{L}~U_{M}A_{L}~C_{M}U_{F12}~U_{M}~U_{F12}~C_{L}~C_{F4}~C_{M}~A_{M}~A_{M}
P-U_{M}~A_{F}~U_{M12}G_{L}~A_{M}G_{F}~A_{L}A_{F}~C_{M4}A_{L}~U_{M}A_{L}~C_{M4}U_{F}~U_{M12}~U_{F}~C_{L4}~C_{F}~C_{M4}~A_{M}~A_{M}
P-U_{M}~A_{F}~U_{M12}G_{L}~A_{M}G_{F}~A_{L}A_{F}~C_{M4}A_{L}~U_{M12}A_{L}~C_{M4}U_{F12}~U_{M12}~U_{F12}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U_{F18}~U_{M}~U_{F18}~C_{L}~C_{F4}~C_{M}~A_{M}~A_{M}
P-U_{M}~A_{F}~U_{M}G_{L}~A_{M}G_{F}~A_{L}A_{F}~C_{M4}A_{L}~U_{M}A_{L}~C_{M4}U_{F}~U_{M18}~U_{F}~C_{L4}~C_{F}~C_{M4}~A_{M}~A_{M}
P-U_{M}~A_{F}~U_{M}G_{L}~A_{M}G_{F}~A_{L}A_{F}~C_{M4}A_{L}~U_{M18}A_{L}~C_{M4}U_{F18}~U_{M18}~U_{F18}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U_{F18}~U_{M}~U_{F18}~C_{L}~C_{F4}~C_{M}~A_{M}~A_{M}
P-U_{M}~A_{F}~U_{M18}G_{L}~A_{M}G_{F}~A_{L}A_{F}~C_{M4}A_{L}~U_{M}A_{L}~C_{M4}U_{F}~U_{M18}~U_{F}~C_{L4}~C_{F}~C_{M4}~A_{M}~A_{M}
P-U_{M}~A_{F}~U_{M18}G_{L}~A_{M}G_{F}~A_{L}A_{F}~C_{M4}A_{L}~U_{M18}A_{L}~C_{M4}U_{F18}~U_{M18}~U_{F18}~C_{L4}~C_{F4}~C_{M4}~A_{M}~A_{M}
P-U_{M}~A_{F}~U_{M18}G_{L}~A_{M}G_{F}~A_{L}A_{F}~C_{M}A_{L}~U_{M}A_{L}~C_{M}U_{F18}~U_{M}~U_{F18}~C_{L}~C_{F4}~C_{M}~A_{M}~A_{M}
P-U_{M}~A_{F}~U_{M18}G_{L}~A_{M}G_{F}~A_{L}A_{F}~C_{M4}A_{L}~U_{M}A_{L}~C_{M4}U_{F}~U_{M18}~U_{F}~C_{L4}~C_{F}~C_{M4}~A_{M}~A_{M}
P-U_{M}~A_{F}~U_{M18}G_{L}~A_{M}G_{F}~A_{L}A_{F}~C_{M4}A_{L}~U_{M18}A_{L}~C_{M4}U_{F18}~U_{M18}~U_{F18}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U~U_{M}~U_{F3}~C_{L}~C_{F4}~C_{M}~A_{M}~A_{M}
P-U_{M}~A_{F}~U_{M}G_{L}~A_{M}G_{F}~A_{L}A_{F}~C_{M4}A_{L}~U_{M}A_{L}~C_{M4}U~U_{M3}~U_{F}~C_{L4}~C_{F}~C_{M4}~A_{M}~A_{M}
P-U_{M}~A_{F}~U_{M}G_{L}~A_{M}G_{F}~A_{L}A_{F}~C_{M4}A_{L}~U_{M3}A_{L}~C_{M4}U~U_{M3}~U_{F3}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U~U_{M}~U_{F3}~C_{L}~C_{F4}~C_{M}~A_{M}~A_{M}
P-U_{M}~A_{F}~U_{M3}G_{L}~A_{M}G_{F}~A_{L}A_{F}~C_{M4}A_{L}~U_{M}A_{L}~C_{M4}U~U_{M3}~U_{F}~C_{L4}~C_{F}~C_{M4}~A_{M}~A_{M}
P-U_{M}~A_{F}~U_{M3}G_{L}~A_{M}G_{F}~A_{L}A_{F}~C_{M4}A_{L}~U_{M3}A_{L}~C_{M4}U~U_{M3}~U_{F3}~C_{L4}~C_{F4}~C_{M4}~A_{M}~A_{M}
P-U_{M}~A_{F}~U_{M3}G_{L}~A_{M}G_{F}~A_{L}A_{F}~C_{M}A_{L}~U_{M}A_{L}~C_{M}U~U_{M}~U_{F3}~C_{L}~C_{F4}~C_{M}~A_{M}~A_{M}
P-U_{M}~A_{F}~U_{M3}G_{L}~A_{M}G_{F}~A_{L}A_{F}~C_{M4}A_{L}~U_{M}A_{L}~C_{M4}U~U_{M3}~U_{F}~C_{L4}~C_{F}~C_{M4}~A_{M}~A_{M}
P-U_{M}~A_{F}~U_{M3}G_{L}~A_{M}G_{F}~A_{L}A_{F}~C_{M4}A_{L}~U_{M3}A_{L}~C_{M4}U~U_{M3}~U_{F3}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U~U_{M}~U_{F12}~C_{L}~C_{F4}~C_{M}~A_{M}~A_{M}
P-U_{M}~A_{F}~U_{M}G_{L}~A_{M}G_{F}~A_{L}A_{F}~C_{M4}A_{L}~U_{M}A_{L}~C_{M4}U~U_{M12}~U_{F}~C_{L4}~C_{F}~C_{M4}~A_{M}~A_{M}
P-U_{M}~A_{F}~U_{M}G_{L}~A_{M}G_{F}~A_{L}A_{F}~C_{M4}A_{L}~U_{M12}A_{L}~C_{M4}U~U_{M12}~U_{F12}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U~U_{M}~U_{F12}~C_{L}~C_{F4}~C_{M}~A_{M}~A_{M}
P-U_{M}~A_{F}~U_{M12}G_{L}~A_{M}G_{F}~A_{L}A_{F}~C_{M4}A_{L}~U_{M}A_{L}~C_{M4}U~U_{M12}~U_{F}~C_{L4}~C_{F}~C_{M4}~A_{M}~A_{M}
P-U_{M}~A_{F}~U_{M12}G_{L}~A_{M}G_{F}~A_{L}A_{F}~C_{M4}A_{L}~U_{M12}A_{L}~C_{M4}U~U_{M12}~U_{F12}~C_{L4}~C_{F4}~C_{M4}~A_{M}~A_{M}
P-U_{M}~A_{F}~U_{M12}G_{L}~A_{M}G_{F}~A_{L}A_{F}~C_{M}A_{L}~U_{M}A_{L}~C_{M}U~U_{M}~U_{F12}~C_{L}~C_{F4}~C_{M}~A_{M}~A_{M}
P-U_{M}~A_{F}~U_{M12}G_{L}~A_{M}G_{F}~A_{L}A_{F}~C_{M4}A_{L}~U_{M}A_{L}~C_{M4}U~U_{M12}~U_{F}~C_{L4}~C_{F}~C_{M4}~A_{M}~A_{M}
P-U_{M}~A_{F}~U_{M12}G_{L}~A_{M}G_{F}~A_{L}A_{F}~C_{M4}A_{L}~U_{M12}A_{L}~C_{M4}U~U_{M12}~U_{F12}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U~U_{M}~U_{F18}~C_{L}~C_{F4}~C_{M}~A_{M}~A_{M}
P-U_{M}~A_{F}~U_{M}G_{L}~A_{M}G_{F}~A_{L}A_{F}~C_{M4}A_{L}~U_{M}A_{L}~C_{M4}U~U_{M18}~U_{F}~C_{L4}~C_{F}~C_{M4}~A_{M}~A_{M}
P-U_{M}~A_{F}~U_{M}G_{L}~A_{M}G_{F}~A_{L}A_{F}~C_{M4}A_{L}~U_{M18}A_{L}~C_{M4}U~U_{M18}~U_{F18}~C_{L4}~C_{F4}~C_{M4}~A_{M}~A_{M}
P-U_{M}~A_{F}~U_{M}G_{L}~A_{M}G_{F}~A_{L}A_{F}~C_{M}A_{L}~U_{M}A_{L}~C_{M}U~U_{M}~U_{F18}~C_{L}~C_{F4}~C_{M}~A_{M}~A_{M}
P-U_{M}~A_{F}~U_{M18}G_{L}~A_{M}G_{F}~A_{L}A_{F}~C_{M4}A_{L}~U_{M}A_{L}~C_{M4}U~U_{M18}~U_{F}~C_{L4}~C_{F}~C_{M4}~A_{M}~A_{M}
P-U_{M}~A_{F}~U_{M18}G_{L}~A_{M}G_{F}~A_{L}A_{F}~C_{M4}A_{L}~U_{M18}A_{L}~C_{M4}U~U_{M18}~U_{F18}~C_{L4}~C_{F4}~C_{M4}~A_{M}~A_{M}
P-U_{M}~A_{F}~U_{M18}G_{L}~A_{M}G_{F}~A_{L}A_{F}~C_{M}A_{L}~U_{M}A_{L}~C_{M}U~U_{M}~U_{F18}~C_{L}~C_{F4}~C_{M}~A_{M}~A_{M}
P-U_{M}~A_{F}~U_{M18}G_{L}~A_{M}G_{F}~A_{L}A_{F}~C_{M4}A_{L}~U_{M}A_{L}~C_{M4}U~U_{M18}~U_{F}~C_{L4}~C_{F}~C_{M4}~A_{M}~A_{M}
P-U_{M}~A_{F}~U_{M18}G_{L}~A_{M}G_{F}~A_{L}A_{F}~C_{M4}A_{L}~U_{M18}A_{L}~C_{M4}U~U_{M18}~U_{F18}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U_{F}~U_{M}~U_{F}~C_{L}~C_{F}~C_{M}~A_{M}~A_{M}
P-U_{M}~A_{F}~U_{M}G_{F}~A_{L}G_{F}~A_{M}A_{L}~C_{M}A_{F}~U_{M}A_{L}~C_{M}U~U_{M}~U_{F}~C_{L}~C_{F}~C_{M}~A_{M}~A_{M}
P-U_{M}~A_{F}~U_{M}G_{F}~A_{L}G_{F}~A_{M}A_{L}~C_{M}A_{F}~U_{M}A_{L}~C_{M}U_{F3}~U_{M}~U_{F3}~C_{L}~C_{F4}~C_{M}~A_{M}~A_{M}
P-U_{M}~A_{F}~U_{M}G_{F}~A_{L}G_{F}~A_{M}A_{L}~C_{M4}A_{F}~U_{M}A_{L}~C_{M4}U_{F}~U_{M3}~U_{F}~C_{L4}~C_{F}~C_{M4}~A_{M}~A_{M}
P-U_{M}~A_{F}~U_{M}G_{F}~A_{L}G_{F}~A_{M}A_{L}~C_{M4}A_{F}~U_{M3}A_{L}~C_{M4}U_{F3}~U_{M3}~U_{F3}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U_{F3}~U_{M}~U_{F3}~C_{L}~C_{F4}~C_{M}~A_{M}~A_{M}
P-U_{M}~A_{F}~U_{M3}G_{F}~A_{L}G_{F}~A_{M}A_{L}~C_{M4}A_{F}~U_{M}A_{L}~C_{M4}U_{F}~U_{M3}~U_{F}~C_{L4}~C_{F}~C_{M4}~A_{M}~A_{M}
P-U_{M}~A_{F}~U_{M3}G_{F}~A_{L}G_{F}~A_{M}A_{L}~C_{M4}A_{F}~U_{M3}A_{L}~C_{M4}U_{F3}~U_{M3}~U_{F3}~C_{L4}~C_{F4}~C_{M4}~A_{M}~A_{M}
P-U_{M}~A_{F}~U_{M3}G_{F}~A_{L}G_{F}~A_{M}A_{L}~C_{M}A_{F}~U_{M}A_{L}~C_{M}U_{F3}~U_{M}~U_{F3}~C_{L}~C_{F4}~C_{M}~A_{M}~A_{M}
P-U_{M}~A_{F}~U_{M3}G_{F}~A_{L}G_{F}~A_{M}A_{L}~C_{M4}A_{F}~U_{M}A_{L}~C_{M4}U_{F}~U_{M3}~U_{F}~C_{L4}~C_{F}~C_{M4}~A_{M}~A_{M}
P-U_{M}~A_{F}~U_{M3}G_{F}~A_{L}G_{F}~A_{M}A_{L}~C_{M4}A_{F}~U_{M3}A_{L}~C_{M4}U_{F3}~U_{M3}~U_{F3}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U_{F12}~U_{M}~U_{F12}~C_{L}~C_{F4}~C_{M}~A_{M}~A_{M}
P-U_{M}~A_{F}~U_{M}G_{F}~A_{L}G_{F}~A_{M}A_{L}~C_{M4}A_{F}~U_{M}A_{L}~C_{M4}U_{F}~U_{M12}~U_{F}~C_{L4}~C_{F}~C_{M4}~A_{M}~A_{M}
P-U_{M}~A_{F}~U_{M}G_{F}~A_{L}G_{F}~A_{M}A_{L}~C_{M4}A_{F}~U_{M12}A_{L}~C_{M4}U_{F12}~U_{M12}~U_{F12}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U_{F12}~U_{M}~U_{F12}~C_{L}~C_{F4}~C_{M}~A_{M}~A_{M}
P-U_{M}~A_{F}~U_{M12}G_{F}~A_{L}G_{F}~A_{M}A_{L}~C_{M4}A_{F}~U_{M}A_{L}~C_{M4}U_{F}~U_{M12}~U_{F}~C_{L4}~C_{F}~C_{M4}~A_{M}~A_{M}
P-U_{M}~A_{F}~U_{M12}G_{F}~A_{L}G_{F}~A_{M}A_{L}~C_{M4}A_{F}~U_{M12}A_{L}~C_{M4}U_{F12}~U_{M12}~U_{F12}~C_{L4}~C_{F4}~C_{M4}~A_{M}~A_{M}
P-U_{M}~A_{F}~U_{M12}G_{F}~A_{L}G_{F}~A_{M}A_{L}~C_{M}A_{F}~U_{M}A_{L}~C_{M}U_{F12}~U_{M}~U_{F12}~C_{L}~C_{F4}~C_{M}~A_{M}~A_{M}
P-U_{M}~A_{F}~U_{M12}G_{F}~A_{L}G_{F}~A_{M}A_{L}~C_{M4}A_{F}~U_{M}A_{L}~C_{M4}U_{F}~U_{M12}~U_{F}~C_{L4}~C_{F}~C_{M4}~A_{M}~A_{M}
P-U_{M}~A_{F}~U_{M12}G_{F}~A_{L}G_{F}~A_{M}A_{L}~C_{M4}A_{F}~U_{M12}A_{L}~C_{M4}U_{F12}~U_{M12}~U_{F12}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U_{F18}~U_{M}~U_{F18}~C_{L}~C_{F4}~C_{M}~A_{M}~A_{M}
P-U_{M}~A_{F}~U_{M}G_{F}~A_{L}G_{F}~A_{M}A_{L}~C_{M4}A_{F}~U_{M}A_{L}~C_{M4}U_{F}~U_{M18}~U_{F}~C_{L4}~C_{F}~C_{M4}~A_{M}~A_{M}
P-U_{M}~A_{F}~U_{M}G_{F}~A_{L}G_{F}~A_{M}A_{L}~C_{M4}A_{F}~U_{M18}A_{L}~C_{M4}U_{F18}~U_{M18}~U_{F18}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U_{F18}~U_{M}~U_{F18}~C_{L}~C_{F4}~C_{M}~A_{M}~A_{M}
P-U_{M}~A_{F}~U_{M18}G_{F}~A_{L}G_{F}~A_{M}A_{L}~C_{M4}A_{F}~U_{M}A_{L}~C_{M4}U_{F}~U_{M18}~U_{F}~C_{L4}~C_{F}~C_{M4}~A_{M}~A_{M}
P-U_{M}~A_{F}~U_{M18}G_{F}~A_{L}G_{F}~A_{M}A_{L}~C_{M4}A_{F}~U_{M18}A_{L}~C_{M4}U_{F18}~U_{M18}~U_{F18}~C_{L4}~C_{F4}~C_{M4}~A_{M}~A_{M}
P-U_{M}~A_{F}~U_{M18}G_{F}~A_{L}G_{F}~A_{M}A_{L}~C_{M}A_{F}~U_{M}A_{L}~C_{M}U_{F18}~U_{M}~U_{F18}~C_{L}~C_{F4}~C_{M}~A_{M}~A_{M}
P-U_{M}~A_{F}~U_{M18}G_{F}~A_{L}G_{F}~A_{M}A_{L}~C_{M4}A_{F}~U_{M}A_{L}~C_{M4}U_{F}~U_{M18}~U_{F}~C_{L4}~C_{F}~C_{M4}~A_{M}~A_{M}
P-U_{M}~A_{F}~U_{M18}G_{F}~A_{L}G_{F}~A_{M}A_{L}~C_{M4}A_{F}~U_{M18}A_{L}~C_{M4}U_{F18}~U_{M18}~U_{F18}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U~U_{M}~U_{F3}~C_{L}~C_{F4}~C_{M}~A_{M}~A_{M}
P-U_{M}~A_{F}~U_{M}G_{F}~A_{L}G_{F}~A_{M}A_{L}~C_{M4}A_{F}~U_{M}A_{L}~C_{M4}U~U_{M3}~U_{F}~C_{L4}~C_{F}~C_{M4}~A_{M}~A_{M}
P-U_{M}~A_{F}~U_{M}G_{F}~A_{L}G_{F}~A_{M}A_{L}~C_{M4}A_{F}~U_{M3}A_{L}~C_{M4}U~U_{M3}~U_{F3}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U~U_{M}~U_{F3}~C_{L}~C_{F4}~C_{M}~A_{M}~A_{M}
P-U_{M}~A_{F}~U_{M3}G_{F}~A_{L}G_{F}~A_{M}A_{L}~C_{M4}A_{F}~U_{M}A_{L}~C_{M4}U~U_{M3}~U_{F}~C_{L4}~C_{F}~C_{M4}~A_{M}~A_{M}
P-U_{M}~A_{F}~U_{M3}G_{F}~A_{L}G_{F}~A_{M}A_{L}~C_{M4}A_{F}~U_{M3}A_{L}~C_{M4}U~U_{M3}~U_{F3}~C_{L4}~C_{F4}~C_{M4}~A_{M}~A_{M}
P-U_{M}~A_{F}~U_{M3}G_{F}~A_{L}G_{F}~A_{M}A_{L}~C_{M}A_{F}~U_{M}A_{L}~C_{M}U~U_{M}~U_{F3}~C_{L}~C_{F4}~C_{M}~A_{M}~A_{M}
P-U_{M}~A_{F}~U_{M3}G_{F}~A_{L}G_{F}~A_{M}A_{L}~C_{M4}A_{F}~U_{M}A_{L}~C_{M4}U~U_{M3}~U_{F}~C_{L4}~C_{F}~C_{M4}~A_{M}~A_{M}
P-U_{M}~A_{F}~U_{M3}G_{F}~A_{L}G_{F}~A_{M}A_{L}~C_{M4}A_{F}~U_{M3}A_{L}~C_{M4}U~U_{M3}~U_{F3}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U~U_{M}~U_{F12}~C_{L}~C_{F4}~C_{M}~A_{M}~A_{M}
P-U_{M}~A_{F}~U_{M}G_{F}~A_{L}G_{F}~A_{M}A_{L}~C_{M4}A_{F}~U_{M}A_{L}~C_{M4}U~U_{M12}~U_{F}~C_{L4}~C_{F}~C_{M4}~A_{M}~A_{M}
P-U_{M}~A_{F}~U_{M}G_{F}~A_{L}G_{F}~A_{M}A_{L}~C_{M4}A_{F}~U_{M12}A_{L}~C_{M4}U~U_{M12}~U_{F12}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U~U_{M}~U_{F12}~C_{L}~C_{F4}~C_{M}~A_{M}~A_{M}
P-U_{M}~A_{F}~U_{M12}G_{F}~A_{L}G_{F}~A_{M}A_{L}~C_{M4}A_{F}~U_{M}A_{L}~C_{M4}U~U_{M12}~U_{F}~C_{L4}~C_{F}~C_{M4}~A_{M}~A_{M}
P-U_{M}~A_{F}~U_{M12}G_{F}~A_{L}G_{F}~A_{M}A_{L}~C_{M4}A_{F}~U_{M12}A_{L}~C_{M4}U~U_{M12}~U_{F12}~C_{L4}~C_{F4}~C_{M4}~A_{M}~A_{M}
P-U_{M}~A_{F}~U_{M12}G_{F}~A_{L}G_{F}~A_{M}A_{L}~C_{M}A_{F}~U_{M}A_{L}~C_{M}U~U_{M}~U_{F12}~C_{L}~C_{F4}~C_{M}~A_{M}~A_{M}
P-U_{M}~A_{F}~U_{M12}G_{F}~A_{L}G_{F}~A_{M}A_{L}~C_{M4}A_{F}~U_{M}A_{L}~C_{M4}U~U_{M12}~U_{F}~C_{L4}~C_{F}~C_{M4}~A_{M}~A_{M}
P-U_{M}~A_{F}~U_{M12}G_{F}~A_{L}G_{F}~A_{M}A_{L}~C_{M4}A_{F}~U_{M12}A_{L}~C_{M4}U~U_{M12}~U_{F12}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U~U_{M}~U_{F18}~C_{L}~C_{F4}~C_{M}~A_{M}~A_{M}
P-U_{M}~A_{F}~U_{M}G_{F}~A_{L}G_{F}~A_{M}A_{L}~C_{M4}A_{F}~U_{M}A_{L}~C_{M4}U~U_{M18}~U_{F}~C_{L4}~C_{F}~C_{M4}~A_{M}~A_{M}
P-U_{M}~A_{F}~U_{M}G_{F}~A_{L}G_{F}~A_{M}A_{L}~C_{M4}A_{F}~U_{M18}A_{L}~C_{M4}U~U_{M18}~U_{F18}~C_{L4}~C_{F4}~C_{M4}~A_{M}~A_{M}
P-U_{M}~A_{F}~U_{M}G_{F}~A_{L}G_{F}~A_{M}A_{L}~C_{M}A_{F}~U_{M}A_{L}~C_{M}U~U_{M}~U_{F18}~C_{L}~C_{F4}~C_{M}~A_{M}~A_{M}
P-U_{M}~A_{F}~U_{M18}G_{F}~A_{L}G_{F}~A_{M}A_{L}~C_{M4}A_{F}~U_{M}A_{L}~C_{M4}U~U_{M18}~U_{F}~C_{L4}~C_{F}~C_{M4}~A_{M}~A_{M}
P-U_{M}~A_{F}~U_{M18}G_{F}~A_{L}G_{F}~A_{M}A_{L}~C_{M4}A_{F}~U_{M18}A_{L}~C_{M4}U~U_{M18}~U_{F18}~C_{L4}~C_{F4}~C_{M4}~A_{M}~A_{M}
P-U_{M}~A_{F}~U_{M18}G_{F}~A_{L}G_{F}~A_{M}A_{L}~C_{M}A_{F}~U_{M}A_{L}~C_{M}U~U_{M}~U_{F18}~C_{L}~C_{F4}~C_{M}~A_{M}~A_{M}
P-U_{M}~A_{F}~U_{M18}G_{F}~A_{L}G_{F}~A_{M}A_{L}~C_{M4}A_{F}~U_{M}A_{L}~C_{M4}U~U_{M18}~U_{F}~C_{L4}~C_{F}~C_{M4}~A_{M}~A_{M}
P-U_{M}~A_{F}~U_{M18}G_{F}~A_{L}G_{F}~A_{M}A_{L}~C_{M4}A_{F}~U_{M18}A_{L}~C_{M4}U~U_{M18}~U_{F18}~C_{L4}~C_{F4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U_{F}~U_{L}~U_{F}~C_{M}~C_{L}~C_{M}~A_{M}~A_{M}
P-U_{M}~A_{F}~U_{M}G_{F}~A_{L}G_{F}~A_{L}A_{F}~C_{M}A_{F}~U_{L}A_{F}~C_{M}U~U_{L}~U_{F}~C_{M}~C_{L}~C_{M}~A_{M}~A_{M}
P-U_{M}~A_{F}~U_{M}G_{F}~A_{L}G_{F}~A_{L}A_{F}~C_{M}A_{F}~U_{L}A_{F}~C_{M}U_{F3}~U_{L}~U_{F3}~C_{M}~C_{L4}~C_{M}~A_{M}~A_{M}
P-U_{M}~A_{F}~U_{M}G_{F}~A_{L}G_{F}~A_{L}A_{F}~C_{M4}A_{F}~U_{L}A_{F}~C_{M4}U_{F}~U_{L3}~U_{F}~C_{M4}~C_{L}~C_{M4}~A_{M}~A_{M}
P-U_{M}~A_{F}~U_{M}G_{F}~A_{L}G_{F}~A_{L}A_{F}~C_{M4}A_{F}~U_{L3}A_{F}~C_{M4}U_{F3}~U_{L3}~U_{F3}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U_{F3}~U_{L}~U_{F3}~C_{M}~C_{L4}~C_{M}~A_{M}~A_{M}
P-U_{M}~A_{F}~U_{M3}G_{F}~A_{L}G_{F}~A_{L}A_{F}~C_{M4}A_{F}~U_{L}A_{F}~C_{M4}U_{F}~U_{L3}~U_{F}~C_{M4}~C_{L}~C_{M4}~A_{M}~A_{M}
P-U_{M}~A_{F}~U_{M3}G_{F}~A_{L}G_{F}~A_{L}A_{F}~C_{M4}A_{F}~U_{L3}A_{F}~C_{M4}U_{F3}~U_{L3}~U_{F3}~C_{M4}~C_{L4}~C_{M4}~A_{M}~A_{M}
P-U_{M}~A_{F}~U_{M3}G_{F}~A_{L}G_{F}~A_{L}A_{F}~C_{M}A_{F}~U_{L}A_{F}~C_{M}U_{F3}~U_{L}~U_{F3}~C_{M}~C_{L4}~C_{M}~A_{M}~A_{M}
P-U_{M}~A_{F}~U_{M3}G_{F}~A_{L}G_{F}~A_{L}A_{F}~C_{M4}A_{F}~U_{L}A_{F}~C_{M4}U_{F}~U_{L3}~U_{F}~C_{M4}~C_{L}~C_{M4}~A_{M}~A_{M}
P-U_{M}~A_{F}~U_{M3}G_{F}~A_{L}G_{F}~A_{L}A_{F}~C_{M4}A_{F}~U_{L3}A_{F}~C_{M4}U_{F3}~U_{L3}~U_{F3}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U_{F12}~U_{L}~U_{F12}~C_{M}~C_{L4}~C_{M}~A_{M}~A_{M}
P-U_{M}~A_{F}~U_{M}G_{F}~A_{L}G_{F}~A_{L}A_{F}~C_{M4}A_{F}~U_{L}A_{F}~C_{M4}U_{F}~U_{L12}~U_{F}~C_{M4}~C_{L}~C_{M4}~A_{M}~A_{M}
P-U_{M}~A_{F}~U_{M}G_{F}~A_{L}G_{F}~A_{L}A_{F}~C_{M4}A_{F}~U_{L12}A_{F}~C_{M4}U_{F12}~U_{L12}~U_{F12}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U_{F12}~U_{L}~U_{F12}~C_{M}~C_{L4}~C_{M}~A_{M}~A_{M}
P-U_{M}~A_{F}~U_{M12}G_{F}~A_{L}G_{F}~A_{L}A_{F}~C_{M4}A_{F}~U_{L}A_{F}~C_{M4}U_{F}~U_{L12}~U_{F}~C_{M4}~C_{L}~C_{M4}~A_{M}~A_{M}
P-U_{M}~A_{F}~U_{M12}G_{F}~A_{L}G_{F}~A_{L}A_{F}~C_{M4}A_{F}~U_{L12}A_{F}~C_{M4}U_{F12}~U_{L12}~U_{F12}~C_{M4}~C_{L4}~C_{M4}~A_{M}~A_{M}
P-U_{M}~A_{F}~U_{M12}G_{F}~A_{L}G_{F}~A_{L}A_{F}~C_{M}A_{F}~U_{L}A_{F}~C_{M}U_{F12}~U_{L}~U_{F12}~C_{M}~C_{L4}~C_{M}~A_{M}~A_{M}
P-U_{M}~A_{F}~U_{M12}G_{F}~A_{L}G_{F}~A_{L}A_{F}~C_{M4}A_{F}~U_{L}A_{F}~C_{M4}U_{F}~U_{L12}~U_{F}~C_{M4}~C_{L}~C_{M4}~A_{M}~A_{M}
P-U_{M}~A_{F}~U_{M12}G_{F}~A_{L}G_{F}~A_{L}A_{F}~C_{M4}A_{F}~U_{L12}A_{F}~C_{M4}U_{F12}~U_{L12}~U_{F12}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U_{F18}~U_{L}~U_{F18}~C_{M}~C_{L4}~C_{M}~A_{M}~A_{M}
P-U_{M}~A_{F}~U_{M}G_{F}~A_{L}G_{F}~A_{L}A_{F}~C_{M4}A_{F}~U_{L}A_{F}~C_{M4}U_{F}~U_{L18}~U_{F}~C_{M4}~C_{L}~C_{M4}~A_{M}~A_{M}
P-U_{M}~A_{F}~U_{M}G_{F}~A_{L}G_{F}~A_{L}A_{F}~C_{M4}A_{F}~U_{L18}A_{F}~C_{M4}U_{F18}~U_{L18}~U_{F18}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U_{F18}~U_{L}~U_{F18}~C_{M}~C_{L4}~C_{M}~A_{M}~A_{M}
P-U_{M}~A_{F}~U_{M18}G_{F}~A_{L}G_{F}~A_{L}A_{F}~C_{M4}A_{F}~U_{L}A_{F}~C_{M4}U_{F}~U_{L18}~U_{F}~C_{M4}~C_{L}~C_{M4}~A_{M}~A_{M}
P-U_{M}~A_{F}~U_{M18}G_{F}~A_{L}G_{F}~A_{L}A_{F}~C_{M4}A_{F}~U_{L18}A_{F}~C_{M4}U_{F18}~U_{L18}~UFL8~C_{M4}~C_{L4}~C_{M4}~A_{M}~A_{M}
P-U_{M}~A_{F}~U_{M18}G_{F}~A_{L}G_{F}~A_{L}A_{F}~C_{M}A_{F}~U_{L}A_{F}~C_{M}U_{F18}~U_{L}~U_{F18}~C_{M}~C_{L4}~C_{M}~A_{M}~A_{M}
P-U_{M}~A_{F}~U_{M18}G_{F}~A_{L}G_{F}~A_{L}A_{F}~C_{M4}A_{F}~U_{L}A_{F}~C_{M4}U_{F}~U_{L18}~U_{F}~C_{M4}~C_{L}~C_{M4}~A_{M}~A_{M}
P-U_{M}~A_{F}~U_{M18}G_{F}~A_{L}G_{F}~A_{L}A_{F}~C_{M4}A_{F}~U_{L18}A_{F}~C_{M4}U_{F18}~U_{L18}~UFL8~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U~U_{L}~U_{F3}~C_{M}~C_{L4}~C_{M}~A_{M}~A_{M}
P-U_{M}~A_{F}~U_{M}G_{F}~A_{L}G_{F}~A_{L}A_{F}~C_{M4}A_{F}~U_{L}A_{F}~C_{M4}U~U_{L3}~U_{F}~C_{M4}~C_{L}~C_{M4}~A_{M}~A_{M}
P-U_{M}~A_{F}~U_{M}G_{F}~A_{L}G_{F}~A_{L}A_{F}~C_{M4}A_{F}~U_{L3}A_{F}~C_{M4}U~U_{L3}~U_{F3}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U~U_{L}~U_{F3}~C_{M}~C_{L4}~C_{M}~A_{M}~A_{M}
P-U_{M}~A_{F}~U_{M3}G_{F}~A_{L}G_{F}~A_{L}A_{F}~C_{M4}A_{F}~U_{L}A_{F}~C_{M4}U~U_{L3}~U_{F}~C_{M4}~C_{L}~C_{M4}~A_{M}~A_{M}
P-U_{M}~A_{F}~U_{M3}G_{F}~A_{L}G_{F}~A_{L}A_{F}~C_{M4}A_{F}~U_{L3}A_{F}~C_{M4}U~U_{L3}~U_{F3}~C_{M4}~C_{L4}~C_{M4}~A_{M}~A_{M}
P-U_{M}~A_{F}~U_{M3}G_{F}~A_{M}G_{F}~A_{L}A_{F}~C_{M}A_{F}~U_{L}A_{F}~C_{M}U~U_{L}~U_{F3}~C_{M}~C_{L4}~C_{M}~A_{M}~A_{M}
P-U_{M}~A_{F}~U_{M3}G_{F}~A_{L}G_{F}~A_{L}A_{F}~C_{M4}A_{F}~U_{L}A_{F}~C_{M4}U~U_{L3}~U_{F}~C_{M4}~C_{L}~C_{M4}~A_{M}~A_{M}
P-U_{M}~A_{F}~U_{M3}G_{F}~A_{L}G_{F}~A_{L}A_{F}~C_{M4}A_{F}~U_{L3}A_{F}~C_{M4}U~U_{L3}~U_{F3}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U~U_{L}~U_{F12}~C_{M}~C_{L4}~C_{M}~A_{M}~A_{M}
P-U_{M}~A_{F}~U_{M}G_{F}~A_{L}G_{F}~A_{L}A_{F}~C_{M4}A_{F}~U_{L}A_{F}~C_{M4}U~U_{L12}~U_{F}~C_{M4}~C_{L}~C_{M4}~A_{M}~A_{M}
P-U_{M}~A_{F}~U_{M}G_{F}~A_{L}G_{F}~A_{L}A_{F}~C_{M4}A_{F}~U_{L12}A_{F}~C_{M4}U~U_{L12}~U_{F12}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U~U_{L}~U_{F12}~C_{M}~C_{L4}~C_{M}~A_{M}~A_{M}
P-U_{M}~A_{F}~U_{M12}G_{F}~A_{L}G_{F}~A_{L}A_{F}~C_{M4}A_{F}~U_{L}A_{F}~C_{M4}U~U_{L12}~U_{F}~C_{M4}~C_{L}~C_{M4}~A_{M}~A_{M}
P-U_{M}~A_{F}~U_{M12}G_{F}~A_{L}G_{F}~A_{L}A_{F}~C_{M4}A_{F}~U_{L12}A_{F}~C_{M4}U~U_{L12}~U_{F12}~C_{M4}~C_{L4}~C_{M4}~A_{M}~A_{M}
P-U_{M}~A_{F}~U_{M12}G_{F}~A_{L}G_{F}~A_{L}A_{F}~C_{M}A_{F}~U_{L}A_{F}~C_{M}U~U_{L}~U_{F12}~C_{M}~C_{L4}~C_{M}~A_{M}~A_{M}
P-U_{M}~A_{F}~U_{M12}G_{F}~A_{L}G_{F}~A_{L}A_{F}~C_{M4}A_{F}~U_{L}A_{F}~C_{M4}U~U_{L12}~U_{F}~C_{M4}~C_{L}~C_{M4}~A_{M}~A_{M}
P-U_{M}~A_{F}~U_{M12}G_{F}~A_{L}G_{F}~A_{L}A_{F}~C_{M4}A_{F}~U_{L12}A_{F}~C_{M4}U~U_{L12}~U_{F12}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U~U_{L}~U_{F18}~C_{M}~C_{L4}~C_{M}~A_{M}~A_{M}
P-U_{M}~A_{F}~U_{M}G_{F}~A_{L}G_{F}~A_{L}A_{F}~C_{M4}A_{F}~U_{L}A_{F}~C_{M4}U~U_{L18}~U_{F}~C_{M4}~C_{L}~C_{M4}~A_{M}~A_{M}
P-U_{M}~A_{F}~U_{M}G_{F}~A_{L}G_{F}~A_{L}A_{F}~C_{M4}A_{F}~U_{L18}A_{F}~C_{M4}U~U_{L18}~U_{F18}~C_{M4}~C_{L4}~C_{M4}~A_{M}~A_{M}
P-U_{M}~A_{F}~U_{M}G_{F}~A_{L}G_{F}~A_{L}A_{F}~C_{M}A_{F}~U_{L}A_{F}~C_{M}U~U_{L}~U_{F18}~C_{M}~C_{L4}~C_{M}~A_{M}~A_{M}
P-U_{M}~A_{F}~U_{M18}G_{F}~A_{L}G_{F}~A_{L}A_{F}~C_{M4}A_{F}~U_{L}A_{F}~C_{M4}U~U_{L18}~U_{F}~C_{M4}~C_{L}~C_{M4}~A_{M}~A_{M}
P-U_{M}~A_{F}~U_{M18}G_{F}~A_{L}G_{F}~A_{L}A_{F}~C_{M4}A_{F}~U_{L18}A_{F}~C_{M4}U~U_{L18}~U_{F18}~C_{M4}~C_{L4}~C_{M4}~A_{M}~A_{M}
P-U_{M}~A_{F}~U_{M18}G_{F}~A_{L}G_{F}~A_{L}A_{F}~C_{M}A_{F}~U_{L}A_{F}~C_{M}U~U_{L}~U_{F18}~C_{M}~C_{L4}~C_{M}~A_{M}~A_{M}
P-U_{M}~A_{F}~U_{M18}G_{F}~A_{L}G_{F}~A_{L}A_{F}~C_{M4}A_{F}~U_{L}A_{F}~C_{M4}U~U_{L18}~U_{F}~C_{M4}~C_{L}~C_{M4}~A_{M}~A_{M}
P-U_{M}~A_{F}~U_{M18}G_{F}~A_{L}G_{F}~A_{L}A_{F}~C_{M4}A_{F}~U_{L18}A_{F}~C_{M4}U~U_{L18}~U_{F18}~C_{M4}~C_{L4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U_{F}~U_{M}~U_{F}~C_{L}~C_{F}~C_{M}~A_{M}~A_{M}
P-U_{M}~A_{L}~U_{M}G_{F}~A_{M}G_{L}~A_{M}A_{F}~C_{L}A_{F}~U_{M}A_{F}~C_{L}U~U_{M}~U_{F}~C_{L}~C_{F}~C_{M}~A_{M}~A_{M}
P-U_{M}~A_{L}~U_{M}G_{F}~A_{M}G_{L}~A_{M}A_{F}~C_{L}A_{F}~U_{M}A_{F}~C_{L}U_{F3}~U_{M}~U_{F3}~C_{L}~C_{F4}~C_{M}~A_{M}~A_{M}
P-U_{M}~A_{L}~U_{M}G_{F}~A_{M}G_{L}~A_{M}A_{F}~C_{L4}A_{F}~U_{M}A_{F}~C_{L4}U_{F}~U_{M3}~U_{F}~C_{L4}~C_{F}~C_{M4}~A_{M}~A_{M}
P-U_{M}~A_{L}~U_{M}G_{F}~A_{M}G_{L}~A_{M}A_{F}~C_{L4}A_{F}~U_{M3}A_{F}~C_{L4}U_{F3}~U_{M3}~U_{F3}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U_{F3}~U_{M}~U_{F3}~C_{L}~C_{F4}~C_{M}~A_{M}~A_{M}
P-U_{M}~A_{L}~U_{M3}G_{F}~A_{M}G_{L}~A_{M}A_{F}~C_{L4}A_{F}~U_{M}A_{F}~C_{L4}U_{F}~U_{M3}~U_{F}~C_{L4}~C_{F}~C_{M4}~A_{M}~A_{M}
P-U_{M}~A_{L}~U_{M3}G_{F}~A_{M}G_{L}~A_{M}A_{F}~C_{L4}A_{F}~U_{M3}A_{F}~C_{L4}U_{F3}~U_{M3}~U_{F3}~C_{L4}~C_{F4}~C_{M4}~A_{M}~A_{M}
P-U_{M}~A_{L}~U_{M3}G_{F}~A_{M}G_{L}~A_{M}A_{F}~C_{L}A_{F}~U_{M}A_{F}~C_{L}U_{F3}~U_{M}~U_{F3}~C_{L}~C_{F4}~C_{M}~A_{M}~A_{M}
P-U_{M}~A_{L}~U_{M3}G_{F}~A_{M}G_{L}~A_{M}A_{F}~C_{L4}A_{F}~U_{M}A_{F}~C_{L4}U_{F}~U_{M3}~U_{F}~C_{L4}~C_{F}~C_{M4}~A_{M}~A_{M}
P-U_{M}~A_{L}~U_{M3}G_{F}~A_{M}G_{L}~A_{M}A_{F}~C_{L4}A_{F}~U_{M3}A_{F}~C_{L4}U_{F3}~U_{M3}~U_{F3}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U_{F12}~U_{M}U_{F12}~C_{L}~C_{F4}~C_{M}~A_{M}~A_{M}
P-U_{M}~A_{L}~U_{M}G_{F}~A_{M}G_{L}~A_{M}A_{F}~C_{L4}A_{F}~U_{M}A_{F}~C_{L4}U_{F}~U_{M12}~U_{F}~C_{L4}~C_{F}~C_{M4}~A_{M}~A_{M}
P-U_{M}~A_{L}~U_{M}G_{F}~A_{M}G_{L}~A_{M}A_{F}~C_{L4}A_{F}~U_{M12}A_{F}~C_{L4}U_{F12}~U_{M12}~U_{F12}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U_{F12}~U_{M}~U_{F12}~C_{L}~C_{F4}~C_{M}~A_{M}~A_{M}
P-U_{M}~A_{L}~U_{M12}G_{F}~A_{M}G_{L}~A_{M}A_{F}~C_{L4}A_{F}~U_{M}A_{F}~C_{L4}U_{F}~U_{M12}~U_{F}~C_{L4}~C_{F}~C_{M4}~A_{M}~A_{M}
P-U_{M}~A_{L}~U_{M12}G_{F}~A_{M}G_{L}~A_{M}A_{F}~C_{L4}A_{F}~U_{M12}A_{F}~C_{L4}U_{F12}~U_{M12}~U_{F12}~C_{L4}~C_{F4}~C_{M4}~A_{M}~A_{M}
P-U_{M}~A_{L}~U_{M12}G_{F}~A_{M}G_{L}~A_{M}A_{F}~C_{L}A_{F}~U_{M}A_{F}~C_{L}U_{F12}~U_{M}~U_{F12}~C_{L}~C_{F4}~C_{M}~A_{M}~A_{M}
P-U_{M}~A_{L}~U_{M12}G_{F}~A_{M}G_{L}~A_{M}A_{F}~C_{L4}A_{F}~U_{M}A_{F}~C_{L4}U_{F}~U_{M12}~U_{F}~C_{L4}~C_{F}~C_{M4}~A_{M}~A_{M}
P-U_{M}~A_{L}~U_{M12}G_{F}~A_{M}G_{L}~A_{M}A_{F}~C_{L4}A_{F}~U_{M12}A_{F}~C_{L4}U_{F12}~U_{M12}~U_{F12}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U_{F18}~U_{M}~U_{F18}~C_{L}~C_{F4}~C_{M}~A_{M}~A_{M}
P-U_{M}~A_{L}~U_{M}G_{F}~A_{M}G_{L}~A_{M}A_{F}~C_{L4}A_{F}~U_{M}A_{F}~C_{L4}U_{F}~U_{M18}~U_{F}~C_{L4}~C_{F}~C_{M4}~A_{M}~A_{M}
P-U_{M}~A_{L}~U_{M}G_{F}~A_{M}G_{L}~A_{M}A_{F}~C_{L4}A_{F}~U_{M18}A_{F}~C_{L4}U_{F18}~U_{M18}~U_{F18}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U_{F18}~U_{M}~U_{F18}~C_{L}~C_{F4}~C_{M}~A_{M}~A_{M}
P-U_{M}~A_{L}~U_{M18}G_{F}~A_{M}G_{L}~A_{M}A_{F}~C_{L4}A_{F}~U_{M}A_{F}~C_{L4}U_{F}~U_{M18}~U_{F}~C_{L4}~C_{F}~C_{M4}~A_{M}~A_{M}
P-U_{M}~A_{L}~U_{M18}G_{F}~A_{M}G_{L}~A_{M}A_{F}~C_{L4}A_{F}~U_{M18}A_{F}~C_{L4}U_{F18}~U_{M18}~U_{F18}~C_{L4}~C_{F4}~C_{M4}~A_{M}~A_{M}
P-U_{M}~A_{L}~U_{M18}G_{F}~A_{M}G_{L}~A_{M}A_{F}~C_{L}A_{F}~U_{M}A_{F}~C_{L}U_{F18}~U_{M}~U_{F18}~C_{L}~C_{F4}~C_{M}~A_{M}~A_{M}
P-U_{M}~A_{L}~U_{M18}G_{F}~A_{M}G_{L}~A_{M}A_{F}~C_{L4}A_{F}~U_{M}A_{F}~C_{L4}U_{F}~U_{M18}~U_{F}~C_{L4}~C_{F}~C_{M4}~A_{M}~A_{M}
P-U_{M}~A_{L}~U_{M18}G_{F}~A_{M}G_{L}~A_{M}A_{F}~C_{L4}A_{F}~U_{M18}A_{F}~C_{L4}U_{F18}~U_{M18}~U_{F18}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U~U_{M}~U_{F3}~C_{L}~C_{F4}~C_{M}~A_{M}~A_{M}
P-U_{M}~A_{L}~U_{M}G_{F}~A_{M}G_{L}~A_{M}A_{F}~C_{L4}A_{F}~U_{M}A_{F}~C_{L4}U~U_{M3}~U_{F}~C_{L4}~C_{F}~C_{M4}~A_{M}~A_{M}
P-U_{M}~A_{L}~U_{M}G_{F}~A_{M}G_{L}~A_{M}A_{F}~C_{L4}A_{F}~U_{M3}A_{F}~C_{L4}U~U_{M3}~U_{F3}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U~U_{M}~U_{F3}~C_{L}~C_{F4}~C_{M}~A_{M}~A_{M}
P-U_{M}~A_{L}~U_{M3}G_{F}~A_{M}G_{L}~A_{M}A_{F}~C_{L4}A_{F}~U_{M}A_{F}~C_{L4}U~U_{M3}~U_{F}~C_{L4}~C_{F}~C_{M4}~A_{M}~A_{M}
P-U_{M}~A_{L}~U_{M3}G_{F}~A_{M}G_{L}~A_{M}A_{F}~C_{L4}A_{F}~U_{M3}A_{F}~C_{L4}U~U_{M3}~U_{F3}~C_{L4}~C_{F4}~C_{M4}~A_{M}~A_{M}
P-U_{M}~A_{L}~U_{M3}G_{F}~A_{M}G_{L}~A_{M}A_{F}~C_{L}A_{F}~U_{M}A_{F}~C_{L}U~U_{M}~U_{F3}~C_{L}~C_{F4}~C_{M}~A_{M}~A_{M}
P-U_{M}~A_{L}~U_{M3}G_{F}~A_{M}G_{L}~A_{M}A_{F}~C_{L4}A_{F}~U_{M}A_{F}~C_{L4}U~U_{M3}~U_{F}~C_{L4}~C_{F}~C_{M4}~A_{M}~A_{M}
P-U_{M}~A_{L}~U_{M3}G_{F}~A_{M}G_{L}~A_{M}A_{F}~C_{L4}A_{F}~U_{M3}A_{F}~C_{L4}U~U_{M3}~U_{F3}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U~U_{M}~U_{F12}~C_{L}~C_{F4}~C_{M}~A_{M}~A_{M}
P-U_{M}~A_{L}~U_{M}G_{F}~A_{M}G_{L}~A_{M}A_{F}~C_{L4}A_{F}~U_{M}A_{F}~C_{L4}U~U_{M12}~U_{F}~C_{L4}~C_{F}~C_{M4}~A_{M}~A_{M}
P-U_{M}~A_{L}~U_{M}G_{F}~A_{M}G_{L}~A_{M}A_{F}~C_{L4}A_{F}~U_{M12}A_{F}~C_{L4}U~U_{M12}~U_{F12}~C_{L4}~C_{F4}~C_{M}~A_{M}~A_{M}
P-U_{M}~A_{L}~U_{M}G_{F}~A_{M}G_{L}~A_{M}A_{F}~C_{L}A_{F}~U_{M}A_{F}~C_{L}U~U_{M}~U_{F12}~C_{L}~C_{F4}~C_{M}~A_{M}~A_{M}
P-U_{M}~A_{L}~U_{M12}G_{F}~A_{M}G_{L}~A_{M}A_{F}~C_{L4}A_{F}~U_{M}A_{F}~C_{L4}U~U_{M12}~U_{F}~C_{L4}~C_{F}~C_{M4}~A_{M}~A_{M}
P-U_{M}~A_{L}~U_{M12}G_{F}~A_{M}G_{L}~A_{M}A_{F}~C_{L4}A_{F}~U_{M12}A_{F}~C_{L4}U~U_{M12}~U_{F12}~C_{L4}~C_{F4}~C_{M4}~A_{M}~A_{M}
P-U_{M}~A_{L}~U_{M12}G_{F}~A_{M}G_{L}~A_{M}A_{F}~C_{L}A_{F}~U_{M}A_{F}~C_{L}U~U_{M}~U_{F12}~C_{L}~C_{F4}~C_{M}~A_{M}~A_{M}
P-U_{M}~A_{L}~U_{M12}G_{F}~A_{M}G_{L}~A_{M}A_{F}~C_{L4}A_{F}~U_{M}A_{F}~C_{L4}U~U_{M12}~U_{F}~C_{L4}~C_{F}~C_{M4}~A_{M}~A_{M}
P-U_{M}~A_{L}~U_{M12}G_{F}~A_{M}G_{L}~A_{M}A_{F}~C_{L4}A_{F}~U_{M12}A_{F}~C_{L4}U~U_{M12}~U_{F12}~C_{L4}~C_{F4}~C_{M4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U~U_{M}~U_{F18}~C_{L}~C_{F4}~C_{M}~A_{M}~A_{M}
P-U_{M}~A_{L}~U_{M}G_{F}~A_{M}G_{L}~A_{M}A_{F}~C_{L4}A_{F}~U_{M}A_{F}~C_{L4}U~U_{M18}~U_{F}~C_{L4}~C_{F}~C_{M4}~A_{M}~A_{M}
P-U_{M}~A_{L}~U_{M}G_{F}~A_{M}G_{L}~A_{M}A_{F}~C_{L4}A_{F}~U_{M18}A_{F}~C_{L4}U~U_{M18}~U_{F18}~C_{L4}~C_{F4}~A_{M}~A_{M}
P-U_{M}~A_{L}~U_{M}G_{F}~A_{M}G_{L}~A_{M}A_{F}~C_{L}A_{F}~U_{M}A_{F}~C_{L}U~U_{M}~U_{F18}~C_{L}~C_{F4}~C_{M}~A_{M}~A_{M}
P-U_{M}~A_{L}~U_{M18}G_{F}~A_{M}G_{L}~A_{M}A_{F}~C_{L4}A_{F}~U_{M}A_{F}~C_{L4}U~U_{M18}~U_{F}~C_{L4}~C_{F}~C_{M4}~A_{M}~A_{M}
P-U_{M}~A_{L}~U_{M18}G_{F}~A_{M}G_{L}~A_{M}A_{F}~C_{L4}A_{F}~U_{M18}A_{F}~C_{L4}U~U_{M18}~U_{F18}~C_{L4}~C_{F4}~C_{M4}~A_{M}~A_{M}
P-U_{M}~A_{L}~U_{M18}G_{F}~A_{M}G_{L}~A_{M}A_{F}~C_{L}A_{F}~U_{M}A_{F}~C_{L}U~U_{M}~U_{F18}~C_{L}~C_{F4}~C_{M}~A_{M}~A_{M}
P-U_{M}~A_{L}~U_{M18}G_{F}~A_{M}G_{L}~A_{M}A_{F}~C_{L4}A_{F}~U_{M}A_{F}~C_{L4}U~U_{M18}~U_{F}~C_{L4}~C_{F}~C_{M4}~A_{M}~A_{M}
P-U_{M}~A_{L}~U_{M18}G_{F}~A_{M}G_{L}~A_{M}A_{F}~C_{L4}A_{F}~U_{M18}A_{F}~C_{L4}U~U_{M18}~U_{F18}~C_{L4}~C_{F4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U_{F}~U_{L}~U_{F}~C_{M}~C_{L}~C_{M}~A_{M}~A_{M}
P-U_{M}~A_{F}~U_{L}G_{F}~A_{L}G_{F}~A_{M}A_{F}~C_{L}A_{F}~U_{M}A_{F}~C_{M}U~U_{L}~U_{F}~C_{M}~C_{L}~C_{M}~A_{M}~A_{M}
P-U_{M}~A_{F}~U_{L}G_{F}~A_{L}G_{F}~A_{M}A_{F}~C_{L}A_{F}~U_{M}A_{F}~C_{M}U_{F3}~U_{L}~U_{F3}~C_{M}~C_{L4}~C_{M}~A_{M}~A_{M}
P-U_{M}~A_{F}~U_{L}G_{F}~A_{L}G_{F}~A_{M}A_{F}~C_{L4}A_{F}~U_{M}A_{F}~C_{M4}U_{F}~U_{L3}~U_{F}~C_{M4}~C_{L}~C_{M4}~A_{M}~A_{M}
P-U_{M}~A_{F}~U_{L}G_{F}~A_{L}G_{F}~A_{M}A_{F}~C_{L4}A_{F}~U_{M3}A_{F}~C_{M4}U_{F3}~U_{L3}~U_{F3}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U_{F3}~U_{L}~U_{F3}~C_{M}~C_{L4}~C_{M}~A_{M}~A_{M}
P-U_{M}~A_{F}~U_{L3}G_{F}~A_{L}G_{F}~A_{M}A_{F}~C_{L4}A_{F}~U_{M}A_{F}~C_{M4}U_{F}~U_{L3}~U_{F}~C_{M4}~C_{L}~C_{M4}~A_{M}~A_{M}
P-U_{M}~A_{F}~U_{L3}G_{F}~A_{L}G_{F}~A_{M}A_{F}~C_{L4}A_{F}~U_{M3}A_{F}~C_{M4}U_{F3}~U_{L3}~U_{F3}~C_{M4}~C_{L4}~C_{M4}~A_{M}~A_{M}
P-U_{M}~A_{F}~U_{L3}G_{F}~A_{L}G_{F}~A_{M}A_{F}~C_{L}A_{F}~U_{M}A_{F}~C_{M}U_{F3}~U_{L}~U_{F3}~C_{M}~C_{L4}~C_{M}~A_{M}~A_{M}
P-U_{M}~A_{F}~U_{L3}G_{F}~A_{L}G_{F}~A_{M}A_{F}~C_{L4}A_{F}~U_{M}A_{F}~C_{M4}U_{F}~U_{L3}~U_{F}~C_{M4}~C_{L}~C_{M4}~A_{M}~A_{M}
P-U_{M}~A_{F}~U_{L3}G_{F}~A_{L}G_{F}~A_{M}A_{F}~C_{L4}A_{F}~U_{M3}A_{F}~C_{M4}U_{F3}~U_{L3}~U_{F3}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U_{F12}~U_{L}~U_{F12}~C_{M}~C_{L4}~C_{M}~A_{M}~A_{M}
P-U_{M}~A_{F}~U_{L}G_{F}~A_{L}G_{F}~A_{M}A_{F}~C_{L4}A_{F}~U_{M}A_{F}~C_{M4}U_{F}~U_{L12}~U_{F}~C_{M4}~C_{L}~C_{M4}~A_{M}~A_{M}
P-U_{M}~A_{F}~U_{L}G_{F}~A_{L}G_{F}~A_{M}A_{F}~C_{L4}A_{F}~U_{M12}A_{F}~C_{M4}U_{F12}~U_{L12}~U_{F12}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U_{F12}~U_{L}~U_{F12}~C_{M}~C_{L4}~C_{M}~A_{M}~A_{M}
P-U_{M}~A_{F}~U_{L12}G_{F}~A_{L}G_{F}~A_{M}A_{F}~C_{L4}A_{F}~U_{M}A_{F}~C_{M4}U_{F}~U_{L12}~U_{F}~C_{M4}~C_{L}~C_{M4}~A_{M}~A_{M}
P-U_{M}~A_{F}~U_{L12}G_{F}~A_{L}G_{F}~A_{M}A_{F}~C_{L4}A_{F}~U_{M12}A_{F}~C_{M4}U_{F12}~U_{L12}~U_{F12}~C_{M4}~C_{L4}~C_{M4}~A_{M}~A_{M}
P-U_{M}~A_{F}~U_{L12}G_{F}~A_{L}G_{F}~A_{M}A_{F}~C_{L}A_{F}~U_{M}A_{F}~C_{M}U_{F12}~U_{L}~U_{F12}~C_{M}~C_{L4}~C_{M}~A_{M}~A_{M}
P-U_{M}~A_{F}~U_{L12}G_{F}~A_{L}G_{F}~A_{M}A_{F}~C_{L4}A_{F}~U_{M}A_{F}~C_{M4}U_{F}~U_{L12}~U_{F}~C_{M4}~C_{L}~C_{M4}~A_{M}~A_{M}
P-U_{M}~A_{F}~U_{L12}G_{F}~A_{L}G_{F}~A_{M}A_{F}~C_{L4}A_{F}~U_{M12}A_{F}~C_{M4}U_{F12}~U_{L12}~U_{F12}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U_{F18}~U_{L}~U_{F18}~C_{M}~C_{L4}~C_{M}~A_{M}~A_{M}
P-U_{M}~A_{F}~U_{L}G_{F}~A_{L}G_{F}~A_{M}A_{F}~C_{L4}A_{F}~U_{M}A_{F}~C_{M4}U_{F}~U_{L18}~U_{F}~C_{M4}~C_{L}~C_{M4}~A_{M}~A_{M}
P-U_{M}~A_{F}~U_{L}G_{F}~A_{L}G_{F}~A_{M}A_{F}~C_{L4}A_{F}~U_{M18}A_{F}~C_{M4}U_{F18}~U_{L18}~U_{F18}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U_{F18}~U_{L}~U_{F18}~C_{M}~C_{L4}~C_{M}~A_{M}~A_{M}
P-U_{M}~A_{F}~U_{L18}G_{F}~A_{L}G_{F}~A_{M}A_{F}~C_{L4}A_{F}~U_{M}A_{F}~C_{M4}U_{F}~U_{L18}~U_{F}~C_{M4}~C_{L}~C_{M4}~A_{M}~A_{M}
P-U_{M}~A_{F}~U_{L18}G_{F}~A_{L}G_{F}~A_{M}A_{F}~C_{L4}A_{F}~U_{M18}A_{F}~C_{M4}U_{F18}~U_{L18}~U_{F18}~C_{M4}~C_{L4}~C_{M4}~A_{M}~A_{M}
P-U_{M}~A_{F}~U_{L18}G_{F}~A_{L}G_{F}~A_{M}A_{F}~C_{L}A_{F}~U_{M}A_{F}~C_{M}U_{F18}~U_{L}~U_{F18}~C_{M}~C_{L4}~C_{M}~A_{M}~A_{M}
P-U_{M}~A_{F}~U_{L18}G_{F}~A_{L}G_{F}~A_{M}A_{F}~C_{L4}A_{F}~U_{M}A_{F}~C_{M4}U_{F}~U_{L18}~U_{F}~C_{M4}~C_{L}~C_{M4}~A_{M}~A_{M}
P-U_{M}~A_{F}~U_{L18}G_{F}~A_{L}G_{F}~A_{M}A_{F}~C_{L4}A_{F}~U_{M18}A_{F}~C_{M4}U_{F18}~U_{L18}~U_{F18}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U~U_{L}~U_{F3}~C_{M}~C_{L4}~C_{M}~A_{M}~A_{M}
P-U_{M}~A_{F}~U_{L}G_{F}~A_{L}G_{F}~A_{M}A_{F}~C_{L4}A_{F}~U_{M}A_{F}~C_{M4}U~U_{L3}~U_{F}~C_{M4}~C_{L}~C_{M4}~A_{M}~A_{M}
P-U_{M}~A_{F}~U_{L}G_{F}~A_{L}G_{F}~A_{M}A_{F}~C_{L4}A_{F}~U_{M3}A_{F}~C_{M4}U~U_{L3}~U_{F3}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U~U_{L}~U_{F3}~C_{M}~C_{L4}~C_{M}~A_{M}~A_{M}
P-U_{M}~A_{F}~U_{L3}G_{F}~A_{L}G_{F}~A_{M}A_{F}~C_{L4}A_{F}~U_{M}A_{F}~C_{M4}U~U_{L3}~U_{F}~C_{M4}~C_{L}~C_{M4}~A_{M}~A_{M}
P-U_{M}~A_{F}~U_{L3}G_{F}~A_{L}G_{F}~A_{M}A_{F}~C_{L4}A_{F}~U_{M3}A_{F}~C_{M4}U~U_{L3}~U_{F3}~C_{M4}~C_{L4}~C_{M4}~A_{M}~A_{M}
P-U_{M}~A_{F}~U_{L3}G_{F}~A_{L}G_{F}~A_{M}A_{F}~C_{L}A_{F}~U_{M}A_{F}~C_{M}U~U_{L}~U_{F3}~C_{M}~C_{L4}~C_{M}~A_{M}~A_{M}
P-U_{M}~A_{F}~U_{L3}G_{F}~A_{L}G_{F}~A_{M}A_{F}~C_{L4}A_{F}~U_{M}A_{F}~C_{M4}U~U_{L3}~U_{F}~C_{M4}~C_{L}~C_{M4}~A_{M}~A_{M}
P-U_{M}~A_{F}~U_{L3}G_{F}~A_{L}G_{F}~A_{M}A_{F}~C_{L4}A_{F}~U_{M3}A_{F}~C_{M4}U~U_{L3}~U_{F3}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U~U_{L}~U_{F12}~C_{M}~C_{L4}~C_{M}~A_{M}~A_{M}
P-U_{M}~A_{F}~U_{L}G_{F}~A_{L}G_{F}~A_{M}A_{F}~C_{L4}A_{F}~U_{M}A_{F}~C_{M4}U~U_{L12}~U_{F}~C_{M4}~C_{L}~C_{M4}~A_{M}~A_{M}
P-U_{M}~A_{F}~U_{L}G_{F}~A_{L}G_{F}~A_{M}A_{F}~C_{L4}A_{F}~U_{M12}A_{F}~C_{M4}U~U_{L12}~U_{F12}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U~U_{L}~U_{F12}~C_{M}~C_{L4}~C_{M}~A_{M}~A_{M}
P-U_{M}~A_{F}~U_{L12}G_{F}~A_{L}G_{F}~A_{M}A_{F}~C_{L4}A_{F}~U_{M}A_{F}~C_{M4}U~U_{L12}~U_{F}~C_{M4}~C_{L}~C_{M4}~A_{M}~A_{M}
P-U_{M}~A_{F}~U_{L12}G_{F}~A_{L}G_{F}~A_{M}A_{F}~C_{L4}A_{F}~U_{M12}A_{F}~C_{M4}U~U_{L12}~U_{F12}~C_{M4}~C_{L4}~C_{M4}~A_{M}~A_{M}
P-U_{M}~A_{F}~U_{L12}G_{F}~A_{L}G_{F}~A_{M}A_{F}~C_{L}A_{F}~U_{M}A_{F}~C_{M}U~U_{L}~U_{F12}~C_{M}~C_{L4}~C_{M}~A_{M}~A_{M}
P-U_{M}~A_{F}~U_{L12}G_{F}~A_{L}G_{F}~A_{M}A_{F}~C_{L4}A_{F}~U_{M}A_{F}~C_{M4}U~U_{L12}~U_{F}~C_{M4}~C_{L}~C_{M4}~A_{M}~A_{M}
P-U_{M}~A_{F}~U_{L12}G_{F}~A_{L}G_{F}~A_{M}A_{F}~C_{L4}A_{F}~U_{M12}A_{F}~C_{M4}U~U_{L12}~U_{F12}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U~U_{L}~U_{F18}~C_{M}~C_{L4}~C_{M}~A_{M}~A_{M}
P-U_{M}~A_{F}~U_{L}G_{F}~A_{L}G_{F}~A_{M}A_{F}~C_{L4}A_{F}~U_{M}A_{F}~C_{M4}U~U_{L18}~U_{F}~C_{M4}~C_{L}~C_{M4}~A_{M}~A_{M}
P-U_{M}~A_{F}~U_{L}G_{F}~A_{L}G_{F}~A_{M}A_{F}~C_{L4}A_{F}~U_{M18}A_{F}~C_{M4}U~U_{L18}~U_{F18}~C_{M4}~C_{L4}~C_{M4}~A_{M}~A_{M}
P-U_{M}~A_{F}~U_{L}G_{F}~A_{L}G_{F}~A_{M}A_{F}~C_{L}A_{F}~U_{M}A_{F}~C_{M}U~U_{L}~U_{F18}~C_{M}~C_{L4}~C_{M}~A_{M}~A_{M}
P-U_{M}~A_{F}~U_{L18}G_{F}~A_{L}G_{F}~A_{M}A_{F}~C_{L4}A_{F}~U_{M}A_{F}~C_{M4}U~U_{L18}~U_{F}~C_{M4}~C_{L}~C_{M4}~A_{M}~A_{M}
P-U_{M}~A_{F}~U_{L18}G_{F}~A_{L}G_{F}~A_{M}A_{F}~C_{L4}A_{F}~U_{M18}A_{F}~C_{M4}U~U_{L18}~U_{F18}~C_{M4}~C_{L4}~C_{M4}~A_{M}~A_{M}
P-U_{M}~A_{F}~U_{L18}G_{F}~A_{L}G_{F}~A_{M}A_{F}~C_{L}A_{F}~U_{M}A_{F}~C_{M}U~U_{L}~U_{F18}~C_{M}~C_{L4}~C_{M}~A_{M}~A_{M}
P-U_{M}~A_{F}~U_{L18}G_{F}~A_{L}G_{F}~A_{M}A_{F}~C_{L4}A_{F}~U_{M}A_{F}~C_{M4}U~U_{L18}~U_{F}~C_{M4}~C_{L}~C_{M4}~A_{M}~A_{M}
P-U_{M}~A_{F}~U_{L18}G_{F}~A_{L}G_{F}~A_{M}A_{F}~C_{L4}A_{F}~U_{M18}A_{F}~C_{M4}U~U_{L18}~U_{F18}~C_{M4}~C_{L4}~C_{M4}~A_{M}~A_{M}

### Table 13

### Examples of Murine SSB ags-siRNA Compositions

### A. Murine SSB Antisense Sequence:

ACAUUAAAGUCUGUUGUCA

Haringsma et al. (Nucleic Acids Res 40: 4125-36, 2012) used this sequence to generate ssRNAi compounds against murine SSB target site 386 including the following:

P-A_{F}C_{F}A_{F}U_{F}U_{F}A_{F}A_{F}A_{F}G_{F}U_{F}C_{F}U_{F}G_{F}U_{F}U_{F}G_{F}U_{F}C_{F}A_{F}U_{M}U_{M}

Any of the 5′-end nucleosides (P-A_{F} or P-A_{M}) shown in the examples of agsRNAi compounds in Tables 13A and 13B can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-A_{F}, P-A_{M}, P-A_{N}, P-A_{L}, P-A_{Z}, P-A_{H}, P-A_{B}, P-A_{K}, P-A_{Q}, P-A_{S}, P-A_{T}, P-A_{W}, P-A_{V}, P-A_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Tables 13A and 13B with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the compounds in Tables 13A and 13B can any of those units and linkages provided for herein for overhang precursors.

B. Examples of Application of AGSD to this Compound:

TABLE 13A
Compositions of Matter Suitable for Use in vitro or in a
Subject with a Protective Carrier (5′-3′)

P-A_{F}C_{F}A_{F}U_{F3}U_{F3}A_{F}A_{F}A_{F}G_{F}U_{F}C_{F}U_{F3}G_{F}U_{F}U_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F}C_{F}U_{F3}G_{F}U_{F}U_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F}C_{F}U_{F3}G_{F}U_{F3}U_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F}C_{F}U_{F3}G_{F}U_{F3}U_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F3}C_{F}U_{F3}G_{F}U_{F3}U_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F3}C_{F}U_{F3}G_{F}U_{F3}U_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F}C_{F4}U_{F3}G_{F}U_{F3}U_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F}C_{F}U_{F3}G_{F}U_{F3}U_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F3}C_{F4}U_{F3}G_{F}U_{F3}U_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F3}C_{F4}U_{F3}G_{F}U_{F3}U_{F3}G_{F}U_{F3}C_{F4}A_{F}A_{F}G_{M}
P-A_{F}C_{F}A_{F}U_{F12}U_{F12}A_{F}A_{F}A_{F}G_{F}U_{F}C_{F}U_{F12}G_{F}U_{F}U_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F}C_{F}U_{F12}G_{F}U_{F}U_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F}C_{F}U_{F12}G_{F}U_{F12}U_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F}C_{F}U_{F12}G_{F}U_{F12}U_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F12}C_{F}U_{F12}G_{F}U_{F12}U_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F12}C_{F}U_{F12}G_{F}U_{F12}U_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F}C_{F4}U_{F12}G_{F}U_{F12}U_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F}C_{F}U_{F12}G_{F}U_{F12}U_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F12}C_{F4}U_{F12}G_{F}U_{F12}U_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F12}C_{F4}U_{F12}G_{F}U_{F12}U_{F12}G_{F}U_{F12}C_{F4}A_{F}A_{F}G_{M}
P-A_{F}C_{F}A_{F}U_{F18}U_{F18}A_{F}A_{F}A_{F}G_{F}U_{F}C_{F}U_{F18}G_{F}U_{F}U_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F}C_{F}U_{F18}G_{F}U_{F}U_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F}C_{F}U_{F18}G_{F}U_{F18}U_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F}C_{F}U_{F18}G_{F}U_{F18}U_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F18}C_{F}U_{F18}G_{F}U_{F18}U_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F18}C_{F}U_{F18}G_{F}U_{F18}U_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F}C_{F4}U_{F18}G_{F}U_{F18}U_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F}C_{F}U_{F18}G_{F}U_{F18}U_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F18}C_{F4}U_{F18}G_{F}U_{F18}U_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F18}C_{F4}U_{F18}G_{F}U_{F18}U_{F18}G_{F}U_{F18}C_{F4}A_{F}A_{F}G_{M}
P-A_{F}C_{F}A_{F}U_{F3}U_{F3}A_{F}A_{F}A_{F}G_{F}U_{F}C_{F}U_{F3}G_{F}UU_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F}C_{F}U_{F3}G_{F}UU_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F}C_{F}U_{F3}G_{F}UU_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F}C_{F}U_{F3}G_{F}UU_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F3}C_{F}U_{F3}G_{F}UU_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F3}C_{F}U_{F3}G_{F}UU_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F}C_{F4}U_{F3}G_{F}UU_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F}C_{F}U_{F3}G_{F}UU_{F3}G_{F}U_{F3}C_{F4}A_{F}A_{F}G_{M}
P-A_{F}C_{F}A_{F}U_{F3}U_{F3}A_{F}A_{F}A_{F}G_{F}U_{F3}C_{F4}U_{F3}G_{F}UU_{F3}G_{F}U_{F3}C_{F}A_{F}A_{F}G_{M}
P-A_{F}C_{F4}A_{F}U_{F3}U_{F3}A_{F}A_{F}A_{F}G_{F}U_{F3}C_{F4}U_{F3}G_{F}UU_{F3}G_{F}U_{F3}C_{F4}A_{F}A_{F}G_{M}
P-A_{F}C_{F}A_{F}U_{F12}U_{F12}A_{F}A_{F}A_{F}G_{F}U_{F}C_{F}U_{F12}G_{F}UU_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F}C_{F}U_{F12}G_{F}UU_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F}C_{F}U_{F12}G_{F}UU_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F}C_{F}U_{F12}G_{F}UU_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F12}C_{F}U_{F12}G_{F}UU_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F12}C_{F}U_{F12}G_{F}UU_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F}C_{F4}U_{F12}G_{F}UU_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F}C_{F}U_{F12}G_{F}UU_{F12}G_{F}U_{F12}C_{F4}A_{F}A_{F}G_{M}
P-A_{F}C_{F}A_{F}U_{F12}U_{F12}A_{F}A_{F}A_{F}G_{F}U_{F12}C_{F4}U_{F12}G_{F}UU_{F12}G_{F}U_{F12}C_{F}A_{F}A_{F}G_{M}
P-A_{F}C_{F4}A_{F}U_{F12}U_{F12}A_{F}A_{F}A_{F}G_{F}U_{F12}C_{F4}U_{F12}G_{F}UU_{F12}G_{F}U_{F12}C_{F4}A_{F}A_{F}G_{M}
P-A_{F}C_{F}A_{F}U_{F18}U_{F18}A_{F}A_{F}A_{F}G_{F}U_{F}C_{F}U_{F18}G_{F}UU_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F}C_{F}U_{F18}G_{F}UU_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F}C_{F}U_{F18}G_{F}UU_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F}C_{F}U_{F18}G_{F}UU_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F18}C_{F}U_{F18}G_{F}UU_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F18}C_{F}U_{F18}G_{F}UU_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F}C_{F4}U_{F18}G_{F}UU_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F}C_{F}U_{F18}G_{F}UU_{F18}G_{F}U_{F18}C_{F4}A_{F}A_{F}G_{M}
P-A_{F}C_{F}A_{F}U_{F18}U_{F18}A_{F}A_{F}A_{F}G_{F}U_{F18}C_{F4}U_{F18}G_{F}UU_{F18}G_{F}U_{F18}C_{F}A_{F}A_{F}G_{M}
P-A_{F}C_{F4}A_{F}U_{F18}U_{F18}A_{F}A_{F}A_{F}G_{F}U_{F18}C_{F4}U_{F18}G_{F}UU_{F18}G_{F}U_{F18}C_{F4}A_{F}A_{F}G_{M}
P-A_{F}C_{F}A_{L}U_{F}U_{F}A_{L}A_{F}A_{F}G_{L}U_{F}C_{F}U_{F}G_{L}U_{F}U_{F}G_{F}U_{L}C_{F}A_{F}A_{F}G_{M}
P-A_{F}C_{F}A_{L}U_{F}U_{F}A_{L}A_{F}A_{F}G_{L}U_{F}C_{F}U_{F}G_{L}UU_{F}G_{F}U_{L}C_{F}A_{F}A_{F}G_{M}
P-A_{F}C_{F4}A_{L}U_{F3}U_{F3}A_{L}A_{F}A_{F}G_{L}U_{F}C_{F}U_{F3}G_{L}U_{F}U_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F}C_{F}U_{F3}G_{L}U_{F3}U_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F}C_{F}U_{F3}G_{L}U_{F3}U_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F3}C_{F}U_{F3}G_{L}U_{F3}U_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F3}C_{F}U_{F3}G_{L}U_{F3}U_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F}C_{F4}U_{F3}G_{L}U_{F3}U_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F}C_{F}U_{F3}G_{L}U_{F3}U_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F3}C_{F4}U_{F3}G_{L}U_{F3}U_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F3}C_{F4}U_{F3}G_{L}U_{F3}U_{F3}G_{F}U_{L3}C_{F4}A_{F}A_{F}G_{M}
P-A_{F}C_{F}A_{L}U_{F12}U_{F12}A_{L}A_{F}A_{F}G_{L}U_{F}C_{F}U_{F12}G_{L}U_{F}U_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F}C_{F}U_{F12}G_{L}U_{F}U_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F}C_{F}U_{F12}G_{L}U_{F12}U_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F}C_{F}U_{F12}G_{L}U_{F12}U_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F12}C_{F}U_{F12}G_{L}U_{F12}U_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F12}C_{F}U_{F12}G_{L}U_{F12}U_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F}C_{F4}U_{F12}G_{L}U_{F12}U_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F}C_{F}U_{F12}G_{L}U_{F12}U_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F12}C_{F4}U_{F12}G_{L}U_{F12}U_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F12}C_{F4}U_{F12}G_{L}U_{F12}U_{F12}G_{F}U_{L12}C_{F4}A_{F}A_{F}G_{M}
P-A_{F}C_{F}A_{L}U_{F18}U_{F18}A_{L}A_{F}A_{F}G_{L}U_{F}C_{F}U_{F18}G_{L}U_{F}U_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F}C_{F}U_{F18}G_{L}U_{F}U_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F}C_{F}U_{F18}G_{L}U_{F18}U_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F}C_{F}U_{F18}G_{L}U_{F18}U_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F18}C_{F}U_{F18}G_{L}U_{F18}U_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F18}C_{F}U_{F18}G_{L}U_{F18}U_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F}C_{F4}U_{F18}G_{L}U_{F18}U_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F}C_{F}U_{F18}G_{L}U_{F18}U_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F18}C_{F4}U_{F18}G_{L}U_{F18}U_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F18}C_{F4}U_{F18}G_{L}U_{F18}U_{F18}G_{F}U_{L18}C_{F4}A_{F}A_{F}G_{M}
P-A_{F}C_{F}A_{L}U_{F3}U_{F3}A_{L}A_{F}A_{F}G_{L}U_{F}C_{F}U_{F3}G_{L}UU_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F}C_{F}U_{F3}G_{L}UU_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F}C_{F}U_{F3}G_{L}UU_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F}C_{F}U_{F3}G_{L}UU_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F3}C_{F}U_{F3}G_{L}UU_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F3}C_{F}U_{F3}G_{L}UU_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F}C_{F4}U_{F3}G_{L}UU_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F}C_{F}U_{F3}G_{L}UU_{F3}G_{F}U_{L3}C_{F4}A_{F}A_{F}G_{M}
P-A_{F}C_{F}A_{L}U_{F3}U_{F3}A_{L}A_{F}A_{F}G_{L}U_{F3}C_{F4}U_{F3}G_{L}UU_{F3}G_{F}U_{L3}C_{F}A_{F}A_{F}G_{M}
P-A_{F}C_{F4}A_{L}U_{F3}U_{F3}A_{L}A_{F}A_{F}G_{L}U_{F3}C_{F4}U_{F3}G_{L}UU_{F3}G_{F}U_{L3}C_{F4}A_{F}A_{F}G_{M}
P-A_{F}C_{F}A_{L}U_{F12}U_{F12}A_{L}A_{F}A_{F}G_{L}U_{F}C_{F}U_{F12}G_{L}UU_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F}C_{F}U_{F12}G_{L}UU_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F}C_{F}U_{F12}G_{L}UU_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F}C_{F}U_{F12}G_{L}UU_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F12}C_{F}U_{F12}G_{L}UU_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F12}C_{F}U_{F12}G_{L}UU_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F}C_{F4}U_{F12}G_{L}UU_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F}C_{F}U_{F12}G_{L}UU_{F12}G_{F}U_{L12}C_{F4}A_{F}A_{F}G_{M}
P-A_{F}C_{F}A_{L}U_{F12}U_{F12}A_{L}A_{F}A_{F}G_{L}U_{F12}C_{F4}U_{F12}G_{L}UU_{F12}G_{F}U_{L12}C_{F}A_{F}A_{F}G_{M}
P-A_{F}C_{F4}A_{L}U_{F12}U_{F12}A_{L}A_{F}A_{F}G_{L}U_{F12}C_{F4}U_{F12}G_{L}UU_{F12}G_{F}U_{L12}C_{F4}A_{F}A_{F}G_{M}
P-A_{F}C_{F}A_{L}U_{F18}U_{F18}A_{L}A_{F}A_{F}G_{L}U_{F}C_{F}U_{F18}G_{L}UU_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F}C_{F}U_{F18}G_{L}UU_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F}C_{F}U_{F18}G_{L}UU_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F}C_{F}U_{F18}G_{L}UU_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F18}C_{F}U_{F18}G_{L}UU_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F18}C_{F}U_{F18}G_{L}UU_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F}C_{F4}U_{F18}G_{L}UU_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F}C_{F}U_{F18}G_{L}UU_{F18}G_{F}U_{L18}C_{F4}A_{F}A_{F}G_{M}
P-A_{F}C_{F}A_{L}U_{F18}U_{F18}A_{L}A_{F}A_{F}G_{L}U_{F18}C_{F4}U_{F18}G_{L}UU_{F18}G_{F}U_{L18}C_{F}A_{F}A_{F}G_{M}
P-A_{F}C_{F4}A_{L}U_{F18}U_{F18}A_{L}A_{F}A_{F}G_{L}U_{F18}C_{F4}U_{F18}G_{L}UU_{F18}G_{F}U_{L18}C_{F4}A_{F}A_{F}G_{M}
P-A_{F}C_{F}A_{F}U_{F}U_{F}A_{L}A_{F}A_{L}G_{F}U_{F}C_{F}U_{F}G_{L}U_{F}U_{F}G_{L}U_{F}C_{L}A_{F}A_{F}G_{M}
P-A_{F}C_{F}A_{F}U_{F}U_{F}A_{L}A_{F}A_{F}G_{L}U_{F}C_{F}U_{F}G_{L}UU_{F}G_{L}U_{F}C_{L}A_{F}A_{F}G_{M}
P-A_{F}C_{F}A_{F}U_{F3}U_{F3}A_{L}A_{F}A_{L}G_{F}U_{F}C_{F}U_{F3}G_{L}U_{F}U_{F3}G_{L}U_{F3}C_{L}A_{F}A_{F}G_{M}
P-A_{F}C_{F4}A_{F}U_{F3}U_{F3}A_{L}A_{F}A_{L}G_{F}U_{F}C_{F}U_{F3}G_{L}U_{F}U_{F3}G_{L}U_{F3}C_{L4}A_{F}A_{F}G_{M}
P-A_{F}C_{F}A_{F}U_{F3}U_{F3}A_{L}A_{F}A_{L}G_{F}U_{F}C_{F}U_{F3}G_{L}U_{F3}U_{F3}G_{L}U_{F3}C_{L}A_{F}A_{F}G_{M}
P-A_{F}C_{F4}A_{F}U_{F3}U_{F3}A_{L}A_{F}A_{L}G_{F}U_{F}C_{F}U_{F3}G_{L}U_{F3}U_{F3}G_{L}U_{F3}C_{L4}A_{F}A_{F}G_{M}
P-A_{F}C_{F}A_{F}U_{F3}U_{F3}A_{L}A_{F}A_{L}G_{F}U_{F3}C_{F}U_{F3}G_{L}U_{F3}U_{F3}G_{L}U_{F3}C_{L}A_{F}A_{F}G_{M}
P-A_{F}C_{F4}A_{F}U_{F3}U_{F3}A_{L}A_{F}A_{L}G_{F}U_{F3}C_{F}U_{F3}G_{L}U_{F3}U_{F3}G_{L}U_{F3}C_{L4}A_{F}A_{F}G_{M}
P-A_{F}C_{F}A_{F}U_{F3}U_{F3}A_{L}A_{F}A_{L}G_{F}U_{F}C_{F4}U_{F3}G_{L}U_{F3}U_{F3}G_{L}U_{F3}C_{L}A_{F}A_{F}G_{M}
P-A_{F}C_{F4}A_{F}U_{F3}U_{F3}A_{L}A_{F}A_{L}G_{F}U_{F}C_{F}U_{F3}G_{L}U_{F3}U_{F3}G_{L}U_{F3}C_{L4}A_{F}A_{F}G_{M}
P-A_{F}C_{F}A_{F}U_{F3}U_{F3}A_{L}A_{F}A_{L}G_{F}U_{F3}C_{F4}U_{F3}G_{L}U_{F3}U_{F3}G_{L}U_{F3}C_{L}A_{F}A_{F}G_{M}
P-A_{F}C_{F4}A_{F}U_{F3}U_{F3}A_{L}A_{F}A_{L}G_{F}U_{F3}C_{F4}U_{F3}G_{L}U_{F3}U_{F3}G_{L}U_{F3}C_{L4}A_{F}A_{F}G_{M}
P-A_{F}C_{F}A_{F}U_{F12}U_{F12}A_{L}A_{F}A_{L}G_{F}U_{F}C_{F}U_{F12}G_{L}U_{F}U_{F12}G_{L}U_{F12}C_{L}A_{F}A_{F}G_{M}
P-A_{F}C_{F4}A_{F}U_{F12}U_{F12}A_{L}A_{F}A_{L}G_{F}U_{F}C_{F}U_{F12}G_{L}U_{F}U_{F12}G_{L}U_{F12}C_{L4}A_{F}A_{F}G_{M}
P-A_{F}C_{F}A_{F}U_{F12}U_{F12}A_{L}A_{F}A_{L}G_{F}U_{F}C_{F}U_{F12}G_{L}U_{F12}U_{F12}G_{L}U_{F12}C_{L}A_{F}A_{F}G_{M}
P-A_{F}C_{F4}A_{F}U_{F12}U_{F12}A_{L}A_{F}A_{L}G_{F}U_{F}C_{F}U_{F12}G_{L}U_{F12}U_{F12}G_{L}U_{F12}C_{L4}A_{F}A_{F}G_{M}
P-A_{F}C_{F}A_{F}U_{F12}U_{F12}A_{L}A_{F}A_{L}G_{F}U_{F12}C_{F}U_{F12}G_{L}U_{F12}U_{F12}G_{L}U_{F12}C_{L}A_{F}A_{F}G_{M}
P-A_{F}C_{F4}A_{F}U_{F12}U_{F12}A_{L}A_{F}A_{L}G_{F}U_{F12}C_{F}U_{F12}G_{L}U_{F12}U_{F12}G_{L}U_{F12}C_{L4}A_{F}A_{F}G_{M}
P-A_{F}C_{F}A_{F}U_{F12}U_{F12}A_{L}A_{F}A_{L}G_{F}U_{F}C_{F4}U_{F12}G_{L}U_{F12}U_{F12}G_{L}U_{F12}C_{L}A_{F}A_{F}G_{M}
P-A_{F}C_{F4}A_{F}U_{F12}U_{F12}A_{L}A_{F}A_{L}G_{F}U_{F}C_{F}U_{F12}G_{L}U_{F12}U_{F12}G_{L}U_{F12}C_{L4}A_{F}A_{F}G_{M}
P-A_{F}C_{F}A_{F}U_{F12}U_{F12}A_{L}A_{F}A_{L}G_{F}U_{F12}C_{F4}U_{F12}G_{L}U_{F12}U_{F12}G_{L}U_{F12}C_{L}A_{F}A_{F}G_{M}
P-A_{F}C_{F4}A_{F}U_{F12}U_{F12}A_{L}A_{F}A_{L}G_{F}U_{F12}C_{F4}U_{F12}G_{L}U_{F12}U_{F12}G_{L}U_{F12}C_{L4}A_{F}A_{F}G_{M}
P-A_{F}C_{F}A_{F}U_{F18}U_{F18}A_{L}A_{F}A_{L}G_{F}U_{F}C_{F}U_{F18}G_{L}U_{F}U_{F18}G_{L}U_{F18}C_{L}A_{F}A_{F}G_{M}
P-A_{F}C_{F4}A_{F}U_{F18}U_{F18}A_{L}A_{F}A_{L}G_{F}U_{F}C_{F}U_{F18}G_{L}U_{F}U_{F18}G_{L}U_{F18}C_{L4}A_{F}A_{F}G_{M}
P-A_{F}C_{F}A_{F}U_{F18}U_{F18}A_{L}A_{F}A_{L}G_{F}U_{F}C_{F}U_{F18}G_{L}U_{F18}U_{F18}G_{L}U_{F18}C_{L}A_{F}A_{F}G_{M}
P-A_{F}C_{F4}A_{F}U_{F18}U_{F18}A_{L}A_{F}A_{L}G_{F}U_{F}C_{F}U_{F18}G_{L}U_{F18}U_{F18}G_{L}U_{F18}C_{L4}A_{F}A_{F}G_{M}
P-A_{F}C_{F}A_{F}U_{F18}U_{F18}A_{L}A_{F}A_{L}G_{F}U_{F18}C_{F}U_{F18}G_{L}U_{F18}U_{F18}G_{L}U_{F18}C_{L}A_{F}A_{F}G_{M}
P-A_{F}C_{F4}A_{F}U_{F18}U_{F18}A_{L}A_{F}A_{L}G_{F}U_{F18}C_{F}U_{F18}G_{L}U_{F18}U_{F18}G_{L}U_{F18}C_{L4}A_{F}A_{F}G_{M}
P-A_{F}C_{F}A_{F}U_{F18}U_{F18}A_{L}A_{F}A_{L}G_{F}U_{F}C_{F4}U_{F18}G_{L}U_{F18}U_{F18}G_{L}U_{F18}C_{L}A_{F}A_{F}G_{M}
P-A_{F}C_{F4}A_{F}U_{F18}U_{F18}A_{L}A_{F}A_{L}G_{F}U_{F}C_{F}U_{F18}G_{L}U_{F18}U_{F18}G_{L}U_{F18}C_{L4}A_{F}A_{F}G_{M}
P-A_{F}C_{F}A_{F}U_{F18}U_{F18}A_{L}A_{F}A_{L}G_{F}U_{F18}C_{F4}U_{F18}G_{L}U_{F18}U_{F18}G_{L}U_{F18}C_{L}A_{F}A_{F}G_{M}
P-A_{F}C_{F4}A_{F}U_{F18}U_{F18}A_{L}A_{F}A_{L}G_{F}U_{F18}C_{F4}U_{F18}G_{L}U_{F18}U_{F18}G_{L}U_{F18}C_{L4}A_{F}A_{F}G_{M}
P-A_{F}C_{F}A_{F}U_{F3}U_{F3}A_{L}A_{F}A_{F}G_{L}U_{F}C_{F}U_{F3}G_{L}UU_{F3}G_{L}U_{F3}C_{L}A_{F}A_{F}G_{M}
P-A_{F}C_{F4}A_{F}U_{F3}U_{F3}A_{L}A_{F}A_{F}G_{L}U_{F}C_{F}U_{F3}G_{L}UU_{F3}G_{L}U_{F3}C_{L4}A_{F}A_{F}G_{M}
P-A_{F}C_{F}A_{F}U_{F3}U_{F3}A_{L}A_{F}A_{F}G_{L}U_{F}C_{F}U_{F3}G_{L}UU_{F3}G_{L}U_{F3}C_{L}A_{F}A_{F}G_{M}
P-A_{F}C_{F4}A_{F}U_{F3}U_{F3}A_{L}A_{F}A_{F}G_{L}U_{F}C_{F}U_{F3}G_{L}UU_{F3}G_{L}U_{F3}C_{L4}A_{F}A_{F}G_{M}
P-A_{F}C_{F}A_{F}U_{F3}U_{F3}A_{L}A_{F}A_{F}G_{L}U_{F3}C_{F}U_{F3}G_{L}UU_{F3}G_{L}U_{F3}C_{L}A_{F}A_{F}G_{M}
P-A_{F}C_{F4}A_{F}U_{F3}U_{F3}A_{L}A_{F}A_{F}G_{L}U_{F3}C_{F}U_{F3}G_{L}UU_{F3}G_{L}U_{F3}C_{L4}A_{F}A_{F}G_{M}
P-A_{F}C_{F}A_{F}U_{F3}U_{F3}A_{L}A_{F}A_{F}G_{L}U_{F}C_{F4}U_{F3}G_{L}UU_{F3}G_{L}U_{F3}C_{L}A_{F}A_{F}G_{M}
P-A_{F}C_{F4}A_{F}U_{F3}U_{F3}A_{L}A_{F}A_{F}G_{L}U_{F}C_{F}U_{F3}G_{L}UU_{F3}G_{L}U_{F3}C_{L4}A_{F}A_{F}G_{M}
P-A_{F}C_{F}A_{F}U_{F3}U_{F3}A_{L}A_{F}A_{F}G_{L}U_{F3}C_{F4}U_{F3}G_{L}UU_{F3}G_{L}U_{F3}C_{L}A_{F}A_{F}G_{M}
P-A_{F}C_{F4}A_{F}U_{F3}U_{F3}A_{L}A_{F}A_{F}G_{L}U_{F3}C_{F4}U_{F3}G_{L}UU_{F3}G_{L}U_{F3}C_{L4}A_{F}A_{F}G_{M}
P-A_{F}C_{F}A_{F}U_{F12}U_{F12}A_{L}A_{F}A_{F}G_{L}U_{F}C_{F}U_{F12}G_{L}UU_{F12}G_{L}U_{F12}C_{L}A_{F}A_{F}G_{M}
P-A_{F}C_{F4}A_{F}U_{F12}U_{F12}A_{L}A_{F}A_{F}G_{L}U_{F}C_{F}U_{F12}G_{L}UU_{F12}G_{L}U_{F12}C_{L4}A_{F}A_{F}G_{M}
P-A_{F}C_{F}A_{F}U_{F12}U_{F12}A_{L}A_{F}A_{F}G_{L}U_{F}C_{F}U_{F12}G_{L}UU_{F12}G_{L}U_{F12}C_{L}A_{F}A_{F}G_{M}
P-A_{F}C_{F4}A_{F}U_{F12}U_{F12}A_{L}A_{F}A_{F}G_{L}U_{F}C_{F}U_{F12}G_{L}UU_{F12}G_{L}U_{F12}C_{L4}A_{F}A_{F}G_{M}
P-A_{F}C_{F}A_{F}U_{F12}U_{F12}A_{L}A_{F}A_{F}G_{L}U_{F12}C_{F}U_{F12}G_{L}UU_{F12}G_{L}U_{F12}C_{L}A_{F}A_{F}G_{M}
P-A_{F}C_{F4}A_{F}U_{F12}U_{F12}A_{L}A_{F}A_{F}G_{L}U_{F12}C_{F}U_{F12}G_{L}UU_{F12}G_{L}U_{F12}C_{L4}A_{F}A_{F}G_{M}
P-A_{F}C_{F}A_{F}U_{F12}U_{F12}A_{L}A_{F}A_{F}G_{L}U_{F}C_{F4}U_{F12}G_{L}UU_{F12}G_{L}U_{F12}C_{L}A_{F}A_{F}G_{M}
P-A_{F}C_{F4}A_{F}U_{F12}U_{F12}A_{L}A_{F}A_{F}G_{L}U_{F}C_{F}U_{F12}G_{L}UU_{F12}G_{L}U_{F12}C_{L4}A_{F}A_{F}G_{M}
P-A_{F}C_{F}A_{F}U_{F12}U_{F12}A_{L}A_{F}A_{F}G_{L}U_{F12}C_{F4}U_{F12}G_{L}UU_{F12}G_{L}U_{F12}C_{L}A_{F}A_{F}G_{M}
P-A_{F}C_{F4}A_{F}U_{F12}U_{F12}A_{L}A_{F}A_{F}G_{L}U_{F12}C_{F4}U_{F12}G_{L}UU_{F12}G_{L}U_{F12}C_{L4}A_{F}A_{F}G_{M}
P-A_{F}C_{F}A_{F}U_{F18}U_{F18}A_{L}A_{F}A_{F}G_{L}U_{F}C_{F}U_{F18}G_{L}UU_{F18}G_{L}U_{F18}C_{L}A_{F}A_{F}G_{M}
P-A_{F}C_{F4}A_{F}U_{F18}U_{F18}A_{L}A_{F}A_{F}G_{L}U_{F}C_{F}U_{F18}G_{L}UU_{F18}G_{L}U_{F18}C_{L4}A_{F}A_{F}G_{M}
P-A_{F}C_{F}A_{F}U_{F18}U_{F18}A_{L}A_{F}A_{F}G_{L}U_{F}C_{F}U_{F18}G_{L}UU_{F18}G_{L}U_{F18}C_{L}A_{F}A_{F}G_{M}
P-A_{F}C_{F4}A_{F}U_{F18}U_{F18}A_{L}A_{F}A_{F}G_{L}U_{F}C_{F}U_{F18}G_{L}UU_{F18}G_{L}U_{F18}C_{L4}A_{F}A_{F}G_{M}
P-A_{F}C_{F}A_{F}U_{F18}U_{F18}A_{L}A_{F}A_{F}G_{L}U_{F18}C_{F}U_{F18}G_{L}UU_{F18}G_{L}U_{F18}C_{L}A_{F}A_{F}G_{M}
P-A_{F}C_{F4}A_{F}U_{F18}U_{F18}A_{L}A_{F}A_{F}G_{L}U_{F18}C_{F}U_{F18}G_{L}UU_{F18}G_{L}U_{F18}C_{L4}A_{F}A_{F}G_{M}
P-A_{F}C_{F}A_{F}U_{F18}U_{F18}A_{L}A_{F}A_{F}G_{L}U_{F}C_{F4}U_{F18}G_{L}UU_{F18}G_{L}U_{F18}C_{L}A_{F}A_{F}G_{M}
P-A_{F}C_{F4}A_{F}U_{F18}U_{F18}A_{L}A_{F}A_{F}G_{L}U_{F}C_{F}U_{F18}G_{L}UU_{F18}G_{L}U_{F18}C_{L4}A_{F}A_{F}G_{M}
P-A_{F}C_{F}A_{F}U_{F18}U_{F18}A_{L}A_{F}A_{F}G_{L}U_{F18}C_{F4}U_{F18}G_{L}UU_{F18}G_{L}U_{F18}C_{L}A_{F}A_{F}G_{M}
P-A_{F}C_{F4}A_{F}U_{F18}U_{F18}A_{L}A_{F}A_{F}G_{L}U_{F18}C_{F4}U_{F18}G_{L}UU_{F18}G_{L}U_{F18}C_{L4}A_{F}A_{F}G_{M}
P-A_{F}C_{F}A_{L}U_{F}U_{F}A_{F}A_{L}A_{F}G_{L}U_{F}C_{F}U_{F}G_{F}U_{F}U_{L}G_{F}U_{L}C_{F}A_{L}A_{F}G_{M}
P-A_{F}C_{F}A_{L}U_{F}U_{F}A_{F}A_{L}A_{F}G_{L}U_{F}C_{F}U_{F}G_{F}UU_{L}G_{F}U_{L}C_{F}A_{F}A_{F}G_{M}
P-A_{F}C_{F}A_{L}U_{F3}U_{F3}A_{F}A_{L}A_{F}G_{L}U_{F}C_{F}U_{F3}G_{F}U_{F}U_{L3}G_{F}U_{L3}C_{F}A_{L}A_{F}G_{M}
P-A_{F}C_{F4}A_{L}U_{F3}U_{F3}A_{F}A_{L}A_{F}G_{L}U_{F}C_{F}U_{F3}G_{F}U_{F}U_{L3}G_{F}U_{L3}C_{F4}A_{L}A_{F}G_{M}
P-A_{F}C_{F}A_{L}U_{F3}U_{F3}A_{F}A_{L}A_{F}G_{L}U_{F}C_{F}U_{F3}G_{F}U_{F3}U_{L3}G_{F}U_{L3}C_{F}A_{L}A_{F}G_{M}
P-A_{F}C_{F4}A_{L}U_{F3}U_{F3}A_{F}A_{L}A_{F}G_{L}U_{F}C_{F}U_{F3}G_{F}U_{F3}U_{L3}G_{F}U_{L3}C_{F4}A_{L}A_{F}G_{M}
P-A_{F}C_{F}A_{L}U_{F3}U_{F3}A_{F}A_{L}A_{F}G_{L}U_{F3}C_{F}U_{F3}G_{F}U_{F3}U_{L3}G_{F}U_{L3}C_{F}A_{L}A_{F}G_{M}
P-A_{F}C_{F4}A_{L}U_{F3}U_{F3}A_{F}A_{L}A_{F}G_{L}U_{F3}C_{F}U_{F3}G_{F}U_{F3}U_{L3}G_{F}U_{L3}C_{F4}A_{L}A_{F}G_{M}
P-A_{F}C_{F}A_{L}U_{F3}U_{F3}A_{F}A_{L}A_{F}G_{L}U_{F}C_{F4}U_{F3}G_{F}U_{F3}U_{L3}G_{F}U_{L3}C_{F}A_{L}A_{F}G_{M}
P-A_{F}C_{F4}A_{L}U_{F3}U_{F3}A_{F}A_{L}A_{F}G_{L}U_{F}C_{F}U_{F3}G_{F}U_{F3}U_{L3}G_{F}U_{L3}C_{F4}A_{L}A_{F}G_{M}
P-A_{F}C_{F}A_{L}U_{F3}U_{F3}A_{F}A_{L}A_{F}G_{L}U_{F3}C_{F4}U_{F3}G_{F}U_{F3}U_{L3}G_{F}U_{L3}C_{F}A_{L}A_{F}G_{M}
P-A_{F}C_{F4}A_{L}U_{F3}U_{F3}A_{F}A_{L}A_{F}G_{L}U_{F3}C_{F4}U_{F3}G_{F}U_{F3}U_{L3}G_{F}U_{L3}C_{F4}A_{L}A_{F}G_{M}
P-A_{F}C_{F}A_{L}U_{F12}U_{F12}A_{F}A_{L}A_{F}G_{L}U_{F}C_{F}U_{F12}G_{F}U_{F}U_{L12}G_{F}U_{L12}C_{F}A_{L}A_{F}G_{M}
P-A_{F}C_{F4}A_{L}U_{F12}U_{F12}A_{F}A_{L}A_{F}G_{L}U_{F}C_{F}U_{F12}G_{F}U_{F}U_{L12}G_{F}U_{L12}C_{F4}A_{L}A_{F}G_{M}
P-A_{F}C_{F}A_{L}U_{F12}U_{F12}A_{F}A_{L}A_{F}G_{L}U_{F}C_{F}U_{F12}G_{F}U_{F12}U_{L12}G_{F}U_{L12}C_{F}A_{L}A_{F}G_{M}
P-A_{F}C_{F4}A_{L}U_{F12}U_{F12}A_{F}A_{L}A_{F}G_{L}U_{F}C_{F}U_{F12}G_{F}U_{F12}U_{L12}G_{F}U_{L12}C_{F4}A_{L}A_{F}G_{M}
P-A_{F}C_{F}A_{L}U_{F12}U_{F12}A_{F}A_{L}A_{F}G_{L}U_{F12}C_{F}U_{F12}G_{F}U_{F12}U_{L12}G_{F}U_{L12}C_{F}A_{L}A_{F}G_{M}
P-A_{F}C_{F4}A_{L}U_{F12}U_{F12}A_{F}A_{L}A_{F}G_{L}U_{F12}C_{F}U_{F12}G_{F}U_{F12}U_{L12}G_{F}U_{L12}C_{F4}A_{L}A_{F}G_{M}
P-A_{F}C_{F}A_{L}U_{F12}U_{F12}A_{F}A_{L}A_{F}G_{L}U_{F}C_{F4}U_{F12}G_{F}U_{F12}U_{L12}G_{F}U_{L12}C_{F}A_{L}A_{F}G_{M}
P-A_{F}C_{F4}A_{L}U_{F12}U_{F12}A_{F}A_{L}A_{F}G_{L}U_{F}C_{F}U_{F12}G_{F}U_{F12}U_{L12}G_{F}U_{L12}C_{F4}A_{L}A_{F}G_{M}
P-A_{F}C_{F}A_{L}U_{F12}U_{F12}A_{F}A_{L}A_{F}G_{L}U_{F12}C_{F4}U_{F12}G_{F}U_{F12}U_{L12}G_{F}U_{L12}C_{F}A_{L}A_{F}G_{M}
P-A_{F}C_{F4}A_{L}U_{F12}U_{F12}A_{F}A_{L}A_{F}G_{L}U_{F12}C_{F4}U_{F12}G_{F}U_{F12}U_{L12}G_{F}U_{L12}C_{F4}A_{L}A_{F}G_{M}
P-A_{F}C_{F}A_{L}U_{F18}U_{F18}A_{F}A_{L}A_{F}G_{L}U_{F}C_{F}U_{F18}G_{F}U_{F}U_{L18}G_{F}U_{L18}C_{F}A_{L}A_{F}G_{M}
P-A_{F}C_{F4}A_{L}U_{F18}U_{F18}A_{F}A_{L}A_{F}G_{L}U_{F}C_{F}U_{F18}G_{F}U_{F}U_{L18}G_{F}U_{L18}C_{F4}A_{L}A_{F}G_{M}
P-A_{F}C_{F}A_{L}U_{F18}U_{F18}A_{F}A_{L}A_{F}G_{L}U_{F}C_{F}U_{F18}G_{F}U_{F18}U_{L18}G_{F}U_{L18}C_{F}A_{L}A_{F}G_{M}
P-A_{F}C_{F4}A_{L}U_{F18}U_{F18}A_{F}A_{L}A_{F}G_{L}U_{F}C_{F}U_{F18}G_{F}U_{F18}U_{L18}G_{F}U_{L18}C_{F4}A_{L}A_{F}G_{M}
P-A_{F}C_{F}A_{L}U_{F18}U_{F18}A_{F}A_{L}A_{F}G_{L}U_{F18}C_{F}U_{F18}G_{F}U_{F18}U_{L18}G_{F}U_{L18}C_{F}A_{L}A_{F}G_{M}
P-A_{F}C_{F4}A_{L}U_{F18}U_{F18}A_{F}A_{L}A_{F}G_{L}U_{F18}C_{F}U_{F18}G_{F}U_{F18}U_{L18}G_{F}U_{L18}C_{F4}A_{L}A_{F}G_{M}
P-A_{F}C_{F}A_{L}U_{F18}U_{F18}A_{F}A_{L}A_{F}G_{L}U_{F}C_{F4}U_{F18}G_{F}U_{F18}U_{L18}G_{F}U_{L18}C_{F}A_{L}A_{F}G_{M}
P-A_{F}C_{F4}A_{L}U_{F18}U_{F18}A_{F}A_{L}A_{F}G_{L}U_{F}C_{F}U_{F18}G_{F}U_{F18}U_{L18}G_{F}U_{L18}C_{F4}A_{L}A_{F}G_{M}
P-A_{F}C_{F}A_{L}U_{F18}U_{F18}A_{F}A_{L}A_{F}G_{L}U_{F18}C_{F4}U_{F18}G_{F}U_{F18}U_{L18}G_{F}U_{L18}C_{F}A_{L}A_{F}G_{M}
P-A_{F}C_{F4}A_{L}U_{F18}U_{F18}A_{F}A_{L}A_{F}G_{L}U_{F18}C_{F4}U_{F18}G_{F}U_{F18}U_{L18}G_{F}U_{L18}C_{F4}A_{L}A_{F}G_{M}
P-A_{F}C_{F}A_{L}U_{F3}U_{F3}A_{F}A_{L}A_{F}G_{L}U_{F}C_{F}U_{F3}G_{F}UU_{L3}G_{F}U_{L3}C_{F}A_{F}A_{F}G_{M}
P-A_{F}C_{F4}A_{L}U_{F3}U_{F3}A_{F}A_{L}A_{F}G_{L}U_{F}C_{F}U_{F3}G_{F}UU_{L3}G_{F}U_{L3}C_{F4}A_{F}A_{F}G_{M}
P-A_{F}C_{F}A_{L}U_{F3}U_{F3}A_{F}A_{L}A_{F}G_{L}U_{F}C_{F}U_{F3}G_{F}UU_{L3}G_{F}U_{L3}C_{F}A_{F}A_{F}G_{M}
P-A_{F}C_{F4}A_{L}U_{F3}U_{F3}A_{F}A_{L}A_{F}G_{L}U_{F}C_{F}U_{F3}G_{F}UU_{L3}G_{F}U_{L3}C_{F4}A_{F}A_{F}G_{M}
P-A_{F}C_{F}A_{L}U_{F3}U_{F3}A_{F}A_{L}A_{F}G_{L}U_{F3}C_{F}U_{F3}G_{F}UU_{L3}G_{F}U_{L3}C_{F}A_{F}A_{F}G_{M}
P-A_{F}C_{F4}A_{L}U_{F3}U_{F3}A_{F}A_{L}A_{F}G_{L}U_{F3}C_{F}U_{F3}G_{F}UU_{L3}G_{F}U_{L3}C_{F4}A_{F}A_{F}G_{M}
P-A_{F}C_{F}A_{L}U_{F3}U_{F3}A_{F}A_{L}A_{F}G_{L}U_{F}C_{F4}U_{F3}G_{F}UU_{L3}G_{F}U_{L3}C_{F}A_{F}A_{F}G_{M}
P-A_{F}C_{F4}A_{L}U_{F3}U_{F3}A_{F}A_{L}A_{F}G_{L}U_{F}C_{F}U_{F3}G_{F}UU_{L3}G_{F}U_{L3}C_{F4}A_{F}A_{F}G_{M}
P-A_{F}C_{F}A_{L}U_{F3}U_{F3}A_{F}A_{L}A_{F}G_{L}U_{F3}C_{F4}U_{F3}G_{F}UU_{L3}G_{F}U_{L3}C_{F}A_{F}A_{F}G_{M}
P-A_{F}C_{F4}A_{L}U_{F3}U_{F3}A_{F}A_{L}A_{F}G_{L}U_{F3}C_{F4}U_{F3}G_{F}UU_{L3}G_{F}U_{L3}C_{F4}A_{F}A_{F}G_{M}
P-A_{F}C_{F}A_{L}U_{F12}U_{F12}A_{F}A_{L}A_{F}G_{L}U_{F}C_{F}U_{F12}G_{F}UU_{L12}G_{F}U_{L12}C_{F}A_{F}A_{F}G_{M}
P-A_{F}C_{F4}A_{L}U_{F12}U_{F12}A_{F}A_{L}A_{F}G_{L}U_{F}C_{F}U_{F12}G_{F}UU_{L12}G_{F}U_{L12}C_{F4}A_{F}A_{F}G_{M}
P-A_{F}C_{F}A_{L}U_{F12}U_{F12}A_{F}A_{L}A_{F}G_{L}U_{F}C_{F}U_{F12}G_{F}UU_{L12}G_{F}U_{L12}C_{F}A_{F}A_{F}G_{M}
P-A_{F}C_{F4}A_{L}U_{F12}U_{F12}A_{F}A_{L}A_{F}G_{L}U_{F}C_{F}U_{F12}G_{F}UU_{L12}G_{F}U_{L12}C_{F4}A_{F}A_{F}G_{M}
P-A_{F}C_{F}A_{L}U_{F12}U_{F12}A_{F}A_{L}A_{F}G_{L}U_{F12}C_{F}U_{F12}G_{F}UU_{L12}G_{F}U_{L12}C_{F}A_{F}A_{F}G_{M}
P-A_{F}C_{F4}A_{L}U_{F12}U_{F12}A_{F}A_{L}A_{F}G_{L}U_{F12}C_{F}U_{F12}G_{F}UU_{L12}G_{F}U_{L12}C_{F4}A_{F}A_{F}G_{M}
P-A_{F}C_{F}A_{L}U_{F12}U_{F12}A_{F}A_{L}A_{F}G_{L}U_{F}C_{F4}U_{F12}G_{F}UU_{L12}G_{F}U_{L12}C_{F}A_{F}A_{F}G_{M}
P-A_{F}C_{F4}A_{L}U_{F12}U_{F12}A_{F}A_{L}A_{F}G_{L}U_{F}C_{F}U_{F12}G_{F}UU_{L12}G_{F}U_{L12}C_{F4}A_{F}A_{F}G_{M}
P-A_{F}C_{F}A_{L}U_{F12}U_{F12}A_{F}A_{L}A_{F}G_{L}U_{F12}C_{F4}U_{F12}G_{F}UU_{L12}G_{F}U_{L12}C_{F}A_{F}A_{F}G_{M}
P-A_{F}C_{F4}A_{L}U_{F12}U_{F12}A_{F}A_{L}A_{F}G_{L}U_{F12}C_{F4}U_{F12}G_{F}UU_{L12}G_{F}U_{L12}C_{F4}A_{F}A_{F}G_{M}
P-A_{F}C_{F}A_{L}U_{F18}U_{F18}A_{F}A_{L}A_{F}G_{L}U_{F}C_{F}U_{F18}G_{F}UU_{L18}G_{F}U_{L18}C_{F}A_{F}A_{F}G_{M}
P-A_{F}C_{F4}A_{L}U_{F18}U_{F18}A_{F}A_{L}A_{F}G_{L}U_{F}C_{F}U_{F18}G_{F}UU_{L18}G_{F}U_{L18}C_{F4}A_{F}A_{F}G_{M}
P-A_{F}C_{F}A_{L}U_{F18}U_{F18}A_{F}A_{L}A_{F}G_{L}U_{F}C_{F}U_{F18}G_{F}UU_{L18}G_{F}U_{L18}C_{F}A_{F}A_{F}G_{M}
P-A_{F}C_{F4}A_{L}U_{F18}U_{F18}A_{F}A_{L}A_{F}G_{L}U_{F}C_{F}U_{F18}G_{F}UU_{L18}G_{F}U_{L18}C_{F4}A_{F}A_{F}G_{M}
P-A_{F}C_{F}A_{L}U_{F18}U_{F18}A_{F}A_{L}A_{F}G_{L}U_{F18}C_{F}U_{F18}G_{F}UU_{L18}G_{F}U_{L18}C_{F}A_{F}A_{F}G_{M}
P-A_{F}C_{F4}A_{L}U_{F18}U_{F18}A_{F}A_{L}A_{F}G_{L}U_{F18}C_{F}U_{F18}G_{F}UU_{L18}G_{F}U_{L18}C_{F4}A_{F}A_{F}G_{M}
P-A_{F}C_{F}A_{L}U_{F18}U_{F18}A_{F}A_{L}A_{F}G_{L}U_{F}C_{F4}U_{F18}G_{F}UU_{L18}G_{F}U_{L18}C_{F}A_{F}A_{F}G_{M}
P-A_{F}C_{F4}A_{L}U_{F18}U_{F18}A_{F}A_{L}A_{F}G_{L}U_{F}C_{F}U_{F18}G_{F}UU_{L18}G_{F}U_{L18}C_{F4}A_{F}A_{F}G_{M}
P-A_{F}C_{F}A_{L}U_{F18}U_{F18}A_{F}A_{L}A_{F}G_{L}U_{F18}C_{F4}U_{F18}G_{F}UU_{L18}G_{F}U_{L18}C_{F}A_{F}A_{F}G_{M}
P-A_{F}C_{F4}A_{L}U_{F18}U_{F18}A_{F}A_{L}A_{F}G_{L}U_{F18}C_{F4}U_{F18}G_{F}UU_{L18}G_{F}U_{L18}C_{F4}A_{F}A_{F}G_{M}
P-A_{F}C_{F}A_{F}U_{L}U_{F}A_{F}A_{F}A_{L}G_{F}U_{F}C_{F}U_{L}G_{F}U_{F}U_{L}G_{F}U_{F}C_{L}A_{F}A_{F}G_{M}
P-A_{F}C_{F}A_{F}U_{L}U_{F}A_{F}A_{F}A_{L}G_{F}U_{F}C_{F}U_{L}G_{F}UU_{L}G_{F}U_{L}C_{F}A_{F}A_{F}G_{M}
P-A_{F}C_{F}A_{F}U_{L3}U_{F3}A_{F}A_{F}A_{L}G_{F}U_{F}C_{F}U_{L3}G_{F}U_{F}U_{L3}G_{F}U_{F3}C_{L}A_{F}A_{F}G_{M}
P-A_{F}C_{F4}A_{F}U_{L3}U_{F3}A_{F}A_{F}A_{L}G_{F}U_{F}C_{F}U_{L3}G_{F}U_{F}U_{L3}G_{F}U_{F3}C_{L4}A_{F}A_{F}G_{M}
P-A_{F}C_{F}A_{F}U_{L3}U_{F3}A_{F}A_{F}A_{L}G_{F}U_{F}C_{F}U_{L3}G_{F}U_{F3}U_{L3}G_{F}U_{F3}C_{L}A_{F}A_{F}G_{M}
P-A_{F}C_{F4}A_{F}U_{L3}U_{F3}A_{F}A_{F}A_{L}G_{F}U_{F}C_{F}U_{L3}G_{F}U_{F3}U_{L3}G_{F}U_{F3}C_{L4}A_{F}A_{F}G_{M}
P-A_{F}C_{F}A_{F}U_{L3}U_{F3}A_{F}A_{F}A_{L}G_{F}U_{F3}C_{F}U_{L3}G_{F}U_{F3}U_{L3}G_{F}U_{F3}C_{L}A_{F}A_{F}G_{M}
P-A_{F}C_{F4}A_{F}U_{L3}U_{F3}A_{F}A_{F}A_{L}G_{F}U_{F3}C_{F}U_{L3}G_{F}U_{F3}U_{L3}G_{F}U_{F3}C_{L4}A_{F}A_{F}G_{M}
P-A_{F}C_{F}A_{F}U_{L3}U_{F3}A_{F}A_{F}A_{L}G_{F}U_{F}C_{F4}U_{L3}G_{F}U_{F3}U_{L3}G_{F}U_{F3}C_{L}A_{F}A_{F}G_{M}
P-A_{F}C_{F4}A_{F}U_{L3}U_{F3}A_{F}A_{F}A_{L}G_{F}U_{F}C_{F}U_{L3}G_{F}U_{F3}U_{L3}G_{F}U_{F3}C_{L4}A_{F}A_{F}G_{M}
P-A_{F}C_{F}A_{F}U_{L3}U_{F3}A_{F}A_{F}A_{L}G_{F}U_{F3}C_{F4}U_{L3}G_{F}U_{F3}U_{L3}G_{F}U_{F3}C_{L}A_{F}A_{F}G_{M}
P-A_{F}C_{F4}A_{F}U_{L3}U_{F3}A_{F}A_{F}A_{L}G_{F}U_{F3}C_{F4}U_{L3}G_{F}U_{F3}U_{L3}G_{F}U_{F3}C_{L4}A_{F}A_{F}G_{M}
P-A_{F}C_{F}A_{F}U_{L12}U_{F12}A_{F}A_{F}A_{L}G_{F}U_{F}C_{F}U_{L12}G_{F}U_{F}U_{L12}G_{F}U_{F12}C_{L}A_{F}A_{F}G_{M}
P-A_{F}C_{F4}A_{F}U_{L12}U_{F12}A_{F}A_{F}A_{L}G_{F}U_{F}C_{F}U_{L12}G_{F}U_{F}U_{L12}G_{F}U_{F12}C_{L4}A_{F}A_{F}G_{M}
P-A_{F}C_{F}A_{F}U_{L12}U_{F12}A_{F}A_{F}A_{L}G_{F}U_{F}C_{F}U_{L12}G_{F}U_{F12}U_{L12}G_{F}U_{F12}C_{L}A_{F}A_{F}G_{M}
P-A_{F}C_{F4}A_{F}U_{L12}U_{F12}A_{F}A_{F}A_{L}G_{F}U_{F}C_{F}U_{L12}G_{F}U_{F12}U_{L12}G_{F}U_{F12}C_{L4}A_{F}A_{F}G_{M}
P-A_{F}C_{F}A_{F}U_{L12}U_{F12}A_{F}A_{F}A_{L}G_{F}U_{F12}C_{F}U_{L12}G_{F}U_{F12}U_{L12}G_{F}U_{F12}C_{L}A_{F}A_{F}G_{M}
P-A_{F}C_{F4}A_{F}U_{L12}U_{F12}A_{F}A_{F}A_{L}G_{F}U_{F12}C_{F}U_{L12}G_{F}U_{F12}U_{L12}G_{F}U_{F12}C_{L4}A_{F}A_{F}G_{M}
P-A_{F}C_{F}A_{F}U_{L12}U_{F12}A_{F}A_{F}A_{L}G_{F}U_{F}C_{F4}U_{L12}G_{F}U_{F12}U_{L12}G_{F}U_{F12}C_{L}A_{F}A_{F}G_{M}
P-A_{F}C_{F4}A_{F}U_{L12}U_{F12}A_{F}A_{F}A_{L}G_{F}U_{F}C_{F}U_{L12}G_{F}U_{F12}U_{L12}G_{F}U_{F12}C_{L4}A_{F}A_{F}G_{M}
P-A_{F}C_{F}A_{F}U_{L12}U_{F12}A_{F}A_{F}A_{L}G_{F}U_{F12}C_{F4}U_{L12}G_{F}U_{F12}U_{L12}G_{F}U_{F12}C_{L}A_{F}A_{F}G_{M}
P-A_{F}C_{F4}A_{F}U_{L12}U_{F12}A_{F}A_{F}A_{L}G_{F}U_{F12}C_{F4}U_{L12}G_{F}U_{F12}U_{L12}G_{F}U_{F12}C_{L4}A_{F}A_{F}G_{M}
P-A_{F}C_{F}A_{F}U_{L18}U_{F18}A_{F}A_{F}A_{L}G_{F}U_{F}C_{F}U_{L18}G_{F}U_{F}U_{L18}G_{F}U_{F18}C_{L}A_{F}A_{F}G_{M}
P-A_{F}C_{F4}A_{F}U_{L18}U_{F18}A_{F}A_{F}A_{L}G_{F}U_{F}C_{F}U_{L18}G_{F}U_{F}U_{L18}G_{F}U_{F18}C_{L4}A_{F}A_{F}G_{M}
P-A_{F}C_{F}A_{F}U_{L18}U_{F18}A_{F}A_{F}A_{L}G_{F}U_{F}C_{F}U_{L18}G_{F}U_{F18}U_{L18}G_{F}U_{F18}C_{L}A_{F}A_{F}G_{M}
P-A_{F}C_{F4}A_{F}U_{L18}U_{F18}A_{F}A_{F}A_{L}G_{F}U_{F}C_{F}U_{L18}G_{F}U_{F18}U_{L18}G_{F}U_{F18}C_{F4}A_{F}A_{F}G_{M}
P-A_{F}C_{F}A_{F}U_{L18}U_{F18}A_{F}A_{F}A_{L}G_{F}U_{F18}C_{F}U_{L18}G_{F}U_{F18}U_{L18}G_{F}U_{F18}C_{L}A_{F}A_{F}G_{M}
P-A_{F}C_{F4}A_{F}U_{L18}U_{F18}A_{F}A_{F}A_{L}G_{F}U_{F18}C_{F}U_{L18}G_{F}U_{F18}U_{L18}G_{F}U_{F18}C_{L4}A_{F}A_{F}G_{M}
P-A_{F}C_{F}A_{F}U_{L18}U_{F18}A_{F}A_{F}A_{L}G_{F}U_{F}C_{F4}U_{L18}G_{F}U_{F18}U_{L18}G_{F}U_{F18}C_{L}A_{F}A_{F}G_{M}
P-A_{F}C_{F4}A_{F}U_{L18}U_{F18}A_{F}A_{F}A_{L}G_{F}U_{F}C_{F}U_{F18}G_{F}U_{F18}U_{L18}G_{F}U_{F18}C_{L4}A_{F}A_{F}G_{M}
P-A_{F}C_{F}A_{F}U_{L18}U_{F18}A_{F}A_{F}A_{L}G_{F}U_{F18}C_{F4}U_{L18}G_{F}U_{F18}U_{L18}G_{F}U_{F18}C_{L}A_{F}A_{F}G_{M}
P-A_{F}C_{F4}A_{F}U_{L18}U_{F18}A_{F}A_{F}A_{L}G_{F}U_{F18}C_{F4}U_{L18}G_{F}U_{F18}U_{L18}G_{F}U_{F18}C_{L4}A_{F}A_{F}G_{M}
P-A_{F}C_{F}A_{F}U_{L3}U_{F3}A_{F}A_{F}A_{L}G_{F}U_{F}C_{F}U_{L3}G_{F}UU_{L3}G_{F}U_{L3}C_{F}A_{F}A_{F}G_{M}
P-A_{F}C_{F4}A_{F}U_{L3}U_{F3}A_{F}A_{F}A_{L}G_{F}U_{F}C_{F}U_{L3}G_{F}UU_{L3}G_{F}U_{L3}C_{F4}A_{F}A_{F}G_{M}
P-A_{F}C_{F}A_{F}U_{L3}U_{F3}A_{F}A_{F}A_{L}G_{F}U_{F}C_{F}U_{L3}G_{F}UU_{L3}G_{F}U_{L3}C_{F}A_{F}A_{F}G_{M}
P-A_{F}C_{F4}A_{F}U_{L3}U_{F3}A_{F}A_{F}A_{L}G_{F}U_{F}C_{F}U_{L3}G_{F}UU_{L3}G_{F}U_{L3}C_{F4}A_{F}A_{F}G_{M}
P-A_{F}C_{F}A_{F}U_{L3}U_{F3}A_{F}A_{F}A_{L}G_{F}U_{F3}C_{F}U_{F3}G_{F}UU_{L3}G_{F}U_{L3}C_{F}A_{F}A_{F}G_{M}
P-A_{F}C_{F4}A_{F}U_{L3}U_{F3}A_{F}A_{F}A_{L}G_{F}U_{F3}C_{F}U_{L3}G_{F}UU_{L3}G_{F}U_{L3}C_{F4}A_{F}A_{F}G_{M}
P-A_{F}C_{F}A_{F}U_{L3}U_{F3}A_{F}A_{F}A_{L}G_{F}U_{F}C_{F4}U_{L3}G_{F}UU_{L3}G_{F}U_{L3}C_{F}A_{F}A_{F}G_{M}
P-A_{F}C_{F4}A_{F}U_{L3}U_{F3}A_{F}A_{F}A_{L}G_{F}U_{F}C_{F}U_{L3}G_{F}UU_{L3}G_{F}U_{L3}C_{F4}A_{F}A_{F}G_{M}
P-A_{F}C_{F}A_{F}U_{L3}U_{F3}A_{F}A_{F}A_{L}G_{F}U_{F3}C_{F4}U_{L3}G_{F}UU_{L3}G_{F}U_{L3}C_{F}A_{F}A_{F}G_{M}
P-A_{F}C_{F4}A_{F}U_{L3}U_{F3}A_{F}A_{F}A_{L}G_{F}U_{F3}C_{F4}U_{L3}G_{F}UU_{L3}G_{F}U_{L3}C_{F4}A_{F}A_{F}G_{M}
P-A_{F}C_{F}A_{F}U_{L12}U_{F12}A_{F}A_{F}A_{L}G_{F}U_{F}C_{F}U_{L12}G_{F}UU_{L12}G_{F}U_{L12}C_{F}A_{F}A_{F}G_{M}
P-A_{F}C_{F4}A_{F}U_{L12}U_{F12}A_{F}A_{F}A_{L}G_{F}U_{F}C_{F}U_{L12}G_{F}UU_{L12}G_{F}U_{L12}C_{F4}A_{F}A_{F}G_{M}
P-A_{F}C_{F}A_{F}U_{L12}U_{F12}A_{F}A_{F}A_{L}G_{F}U_{F}C_{F}U_{L12}G_{F}UU_{L12}G_{F}U_{L12}C_{F}A_{F}A_{F}G_{M}
P-A_{F}C_{F4}A_{F}U_{L12}U_{F12}A_{F}A_{F}A_{L}G_{F}U_{F}C_{F}U_{L12}G_{F}UU_{L12}G_{F}U_{L12}C_{F4}A_{F}A_{F}G_{M}
P-A_{F}C_{F}A_{F}U_{L12}U_{F12}A_{F}A_{F}A_{L}G_{F}U_{F12}C_{F}U_{L12}G_{F}UU_{L12}G_{F}U_{L12}C_{F}A_{F}A_{F}G_{M}
P-A_{F}C_{F4}A_{F}U_{L12}U_{F12}A_{F}A_{F}A_{L}G_{F}U_{F12}C_{F}U_{L12}G_{F}UU_{L12}G_{F}U_{L12}C_{F4}A_{F}A_{F}G_{M}
P-A_{F}C_{F}A_{F}U_{L12}U_{F12}A_{F}A_{F}A_{L}G_{F}U_{F}C_{F4}U_{L12}G_{F}UU_{L12}G_{F}U_{L12}C_{F}A_{F}A_{F}G_{M}
P-A_{F}C_{F4}A_{F}U_{L12}U_{F12}A_{F}A_{F}A_{L}G_{F}U_{F}C_{F}U_{L12}G_{F}UU_{L12}G_{F}U_{L12}C_{F4}A_{F}A_{F}G_{M}
P-A_{F}C_{F}A_{F}U_{L12}U_{F12}A_{F}A_{F}A_{L}G_{F}U_{F12}C_{F4}U_{L12}G_{F}UU_{L12}G_{F}U_{L12}C_{F}A_{F}A_{F}G_{M}
P-A_{F}C_{F4}A_{F}U_{L12}U_{F12}A_{F}A_{F}A_{L}G_{F}U_{F12}C_{F4}U_{L12}G_{F}UU_{L12}G_{F}U_{L12}C_{F4}A_{F}A_{F}G_{M}
P-A_{F}C_{F}A_{F}U_{L18}U_{F18}A_{F}A_{F}A_{L}G_{F}U_{F}C_{F}U_{L18}G_{F}UU_{L18}G_{F}U_{L18}C_{F}A_{F}A_{F}G_{M}
P-A_{F}C_{F4}A_{F}U_{L18}U_{F18}A_{F}A_{F}A_{L}G_{F}U_{F}C_{F}U_{L18}G_{F}UU_{L18}G_{F}U_{L18}C_{F4}A_{F}A_{F}G_{M}
P-A_{F}C_{F}A_{F}U_{L18}U_{F18}A_{F}A_{F}A_{L}G_{F}U_{F}C_{F}U_{L18}G_{F}UU_{L18}G_{F}U_{L18}C_{F}A_{F}A_{F}G_{M}
P-A_{F}C_{F4}A_{F}U_{L18}U_{F18}A_{F}A_{F}A_{L}G_{F}U_{F}C_{F}U_{L18}G_{F}UU_{L18}G_{F}U_{L18}C_{F4}A_{F}A_{F}G_{M}
P-A_{F}C_{F}A_{F}U_{L18}U_{F18}A_{F}A_{F}A_{L}G_{F}U_{F18}C_{F}U_{L18}G_{F}UU_{L18}G_{F}U_{L18}C_{F}A_{F}A_{F}G_{M}
P-A_{F}C_{F4}A_{F}U_{L18}U_{F18}A_{F}A_{F}A_{L}G_{F}U_{F18}C_{F}U_{L18}G_{F}UU_{L18}G_{F}U_{L18}C_{F4}A_{F}A_{F}G_{M}
P-A_{F}C_{F}A_{F}U_{L18}U_{F18}A_{F}A_{F}A_{L}G_{F}U_{F}C_{F4}U_{L18}G_{F}UU_{L18}G_{F}U_{L18}C_{F}A_{F}A_{F}G_{M}
P-A_{F}C_{F4}A_{F}U_{L18}U_{F18}A_{F}A_{F}A_{L}G_{F}U_{F}C_{F}U_{L18}G_{F}UU_{L18}G_{F}U_{L18}C_{F4}A_{F}A_{F}G_{M}
P-A_{F}C_{F}A_{F}U_{L18}U_{F18}A_{F}A_{F}A_{L}G_{F}U_{F18}C_{F4}U_{L18}G_{F}UU_{L18}G_{F}U_{L18}C_{F}A_{F}A_{F}G_{M}
P-A_{F}C_{F4}A_{F}U_{L18}U_{F18}A_{F}A_{F}A_{L}G_{F}U_{F18}C_{F4}U_{L18}G_{F}UU_{L18}G_{F}U_{L18}C_{F4}A_{F}A_{F}G_{M}

TABLE 13B
Compositions of Matter Suitable for Administration to a Subject
with or without a Protective Carrier and for Any Other use
as Provided for Herein (5′-3′)

P-A_{M}~C_{F}~A_{M}U_{F3}~U_{M3}A_{F}~A_{M}A_{F}~G_{M}U_{F}~C_{M}U_{F3}~G_{M}U_{F}~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F}~C_{M}U_{F3}~G_{M}U_{F}~U_{M3}~G_{F}~U_{M3}~C_{F4-}A_{M}~A_{F}~G_{M}
P-A_{M}~C_{F}~A_{M}U_{F3}~U_{M3}A_{F}~A_{M}A_{F}~G_{M}U_{F}~C_{M}U_{F3}~G_{M}U_{F3}~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F}~C_{M}U_{F3}~G_{M}U_{F3}~U_{M3}~G_{F}~U_{M3}~C_{F4}~A_{M}~A_{F}~G_{M}
P-A_{M}~C_{F}~A_{M}U_{F3}~U_{M3}A_{F}~A_{M}A_{F}~G_{M}U_{F3}~C_{M}U_{F3}~G_{M}U_{F3}~U_{M3}~G_{F}~U_{M3}~C_{F-}A_{M}~A_{F}~G_{M}
P-A_{M}~C_{F4}~A_{M}U_{F3}~U_{M3}A_{F}~A_{M}A_{F}~G_{M}U_{F3}~C_{M}U_{F3}~G_{M}U_{F3}~U_{M3}~G_{F}~U_{M3}~C_{F4}~A_{M}~A_{F}~G_{M}
P-A_{M}~C_{F}~A_{M}U_{F3}~U_{M3}A_{F}~A_{M}A_{F}~G_{M}U_{F}~C_{M4}U_{F3}~G_{M}U_{F3}~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F}~C_{M}U_{F3}~G_{M}U_{F3}~U_{M3}~G_{F}~U_{M3}~C_{F4}~A_{M}~A_{F}~G_{M}
P-A_{M}~C_{F}~A_{M}U_{F3}~U_{M3}A_{F}~A_{M}A_{F}~G_{M}U_{F3}~C_{M4}U_{F3}~G_{M}U_{F3}~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F3}~C_{M4}U_{F3}~G_{M}U_{F3}~U_{M3}~G_{F}~U_{M3}~C_{F4}~A_{M}~A_{F}~G_{M}
P-A_{M}~C_{F}~A_{M}U_{F12}~U_{M12}A_{F}~A_{M}A_{F}~G_{M}U_{F}~C_{M}U_{F12}~G_{M}U_{F}~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F}~C_{M}U_{F12}~G_{M}U_{F}~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F}~C_{M}U_{F12}~G_{M}U_{F12}~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F}~C_{M}U_{F12}~G_{M}U_{F12}~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F12}~C_{M}U_{F12}~G_{M}U_{F12}~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F12}~C_{M}U_{F12}~G_{M}U_{F12}~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F}~C_{M4}U_{F12}~G_{M}U_{F12}~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F}~C_{M}U_{F12}~G_{M}U_{F12}~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F12}~C_{M4}U_{F12}~G_{M}U_{F12}~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F12}~C_{M4}U_{F12}~G_{M}U_{F12}~U_{M12}~G_{F}~U_{M12}~C_{F4}~A_{M}~A_{F}~G_{M}
P-A_{M}~C_{F}~A_{M}U_{F18}~U_{M18}A_{F}~A_{M}A_{F}~G_{M}U_{F}~C_{M}U_{F18}~G_{M}U_{F}~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F}~C_{M}U_{F18}~G_{M}U_{F}~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F}~C_{M}U_{F18}~G_{M}U_{F18}~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F}~C_{M}U_{F18}~G_{M}U_{F18}~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F18}~C_{M}U_{F18}~G_{M}U_{F18}~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F18}~C_{M}U_{F18}~G_{M}U_{F18}~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F}~C_{M4}U_{F18}~G_{M}U_{F18}~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F}~C_{M}U_{F18}~G_{M}U_{F18}~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F18}~C_{M4}U_{F18}~G_{M}U_{F18}~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F18}~C_{M4}U_{F18}~G_{M}U_{F18}~U_{M18}~G_{F}~U_{M18}~C_{F4}~A_{M}~A_{F}~G_{M}
P-A_{M}~C_{F}~A_{M}U_{F3}~U_{M3}A_{F}~A_{M}A_{F}~G_{M}U_{F}~C_{M}U_{F3}~G_{M}U~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F}~C_{M}U_{F3}~G_{M}U~U_{M3}~G_{F}~U_{M3}~C_{F4}~A_{M}~A_{F}~G_{M}
P-A_{M}~C_{F}~A_{M}U_{F3}~U_{M3}A_{F}~A_{M}A_{F}~G_{M}U_{F}~C_{M}U_{F3}~G_{M}U~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F}~C_{M}U_{F3}~G_{M}U~U_{M3}~G_{F}~U_{M3}~C_{F4}~A_{M}~A_{F}~G_{M}
P-A_{M}~C_{F}~A_{M}U_{F3}~U_{M3}A_{F}~A_{M}A_{F}~G_{M}U_{F3}~C_{M}U_{F3}~G_{M}U~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F3}~C_{M}U_{F3}~G_{M}U~U_{M3}~G_{F}~U_{M3}~C_{F4}~A_{M}~A_{F}~G_{M}
P-A_{M}~C_{F}~A_{M}U_{F3}~U_{M3}A_{F}~A_{M}A_{F}~G_{M}U_{F}~C_{M4}U_{F3}~G_{M}U~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F}~C_{M}U_{F3}~G_{M}U~U_{M3}~G_{F}~U_{M3}~C_{F4}~A_{M}~A_{F}~G_{M}
P-A_{M}~C_{F}~A_{M}U_{F3}~U_{M3}A_{F}~A_{M}A_{F}~G_{M}U_{F3}~C_{M4}U_{F3}~G_{M}U~U_{M3}~G_{F}~U_{M3}~C_{F}~A_{M}~A_{F}~G_{M}
P-A_{M}~C_{F4}~A_{M}U_{F3}~U_{M3}A_{F}~A_{M}A_{F}~G_{M}U_{F3}~C_{M4}U_{F3}~G_{M}U~U_{M3}~G_{F}~U_{M3}~C_{F4}~A_{M}~A_{F}~G_{M}
P-A_{M}~C_{F}~A_{M}U_{F12}~U_{M12}A_{F}~A_{M}A_{F}~G_{M}U_{F}~C_{M}U_{F12}~G_{M}U~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F}~C_{M}U_{F12}~G_{M}U~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F}~C_{M}U_{F12}~G_{M}U~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F}~C_{M}U_{F12}~G_{M}U~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F12}~C_{M}U_{F12}~G_{M}U~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F12}~C_{M}U_{F12}~G_{M}U~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F}~C_{M4}U_{F12}~G_{M}U~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F}~C_{M}U_{F12}~G_{M}U~U_{M12}~G_{F}~U_{M12}~C_{F4}~A_{M}~A_{F}~G_{M}
P-A_{M}~C_{F}~A_{M}U_{F12}~U_{M12}A_{F}~A_{M}A_{F}~G_{M}U_{F12}~C_{M4}U_{F12}~G_{M}U~U_{M12}~G_{F}~U_{M12}~C_{F}~A_{M}~A_{F}~G_{M}
P-A_{M}~C_{F4}~A_{M}U_{F12}~U_{M12}A_{F}~A_{M}A_{F}~G_{M}U_{F12}~C_{M4}U_{F12}~G_{M}U~U_{M12}~G_{F}~U_{M12}~C_{F4}~A_{M}~A_{F}~G_{M}
P-A_{M}~C_{F}~A_{M}U_{F18}~U_{M18}A_{F}~A_{M}A_{F}~G_{M}U_{F}~C_{M}U_{F18}~G_{M}U~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F}~C_{M}U_{F18}~G_{M}U~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F}~C_{M}U_{F18}~G_{M}U~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F}~C_{M}U_{F18}~G_{M}U~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F18}~C_{M}U_{F18}~G_{M}U~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F18}~C_{M}U_{F18}~G_{M}U~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F}~C_{M4}U_{F18}~G_{M}U~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F}~C_{M}U_{F18}~G_{M}U~U_{M18}~G_{F}~U_{M18}~C_{F4}~A_{M}~A_{F}~G_{M}
P-A_{M}~C_{F}~A_{M}U_{F18}~U_{M18}A_{F}~A_{M}A_{F}~G_{M}U_{F18}~C_{M4}U_{F18}~G_{M}U~U_{M18}~G_{F}~U_{M18}~C_{F}~A_{M}~A_{F}~G_{M}
P-A_{M}~C_{F4}~A_{M}U_{F18}~U_{M18}A_{F}~A_{M}A_{F}~G_{M}U_{F18}~C_{M4}U_{F18}~G_{M}U~U_{M18}~G_{F}~U_{M18}~C_{F4}~A_{M}~A_{F}~G_{M}
P-A_{M}~C_{F}~A_{L}U_{F}~U_{M}A_{L}~A_{M}A_{F}~G_{L}U_{F}~C_{M}U_{F}~G_{L}U_{F}~U_{M}~G_{F}~U_{L}~C_{F}~A_{M}~A_{F}~G_{M}
P-A_{M}~C_{F}~A_{L}U_{F}~U_{M}A_{L}~A_{M}A_{F}~G_{L}U_{F}~C_{M}U_{F}~G_{L}U~U_{M}~G_{F}~U_{L}~C_{F}~A_{M}~A_{F}~G_{M}
P-A_{M}~C_{F}~A_{L}U_{F3}~U_{M3}A_{L}~A_{M}A_{F}~G_{L}U_{F}~C_{M}U_{F3}~G_{L}U_{F}~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F}~C_{M}U_{F3}~G_{L}U_{F}~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F}~C_{M}U_{F3}~G_{L}U_{F3}~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F}~C_{M}U_{F3}~G_{L}U_{F3}~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F3}~C_{M}U_{F3}~G_{L}U_{F3}~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F3}~C_{M}U_{F3}~G_{L}U_{F3}~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F}~C_{M4}U_{F3}~G_{L}U_{F3}~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F}~C_{M}U_{F3}~G_{L}U_{F3}~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F3}~C_{M4}U_{F3}~G_{L}U_{F3}~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F3}~C_{M4}U_{F3}~G_{L}U_{F3}~U_{M3}~G_{F}~U_{L3}~C_{F4}~A_{M}~A_{F}~G_{M}
P-A_{M}~C_{F}~A_{L}U_{F12}~U_{M12}A_{L}~A_{M}A_{F}~G_{L}U_{F}~C_{M}U_{F12}~G_{L}U_{F}~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F}~C_{M}U_{F12}~G_{L}U_{F}~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F}~C_{M}U_{F12}~G_{L}U_{F12}~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F}~C_{M}U_{F12}~G_{L}U_{F12}~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F12}~C_{M}U_{F12}~G_{L}U_{F12}~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F12}~C_{M}U_{F12}~G_{L}U_{F12}~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F}~C_{M4}U_{F12}~G_{L}U_{F12}~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F}~C_{M}U_{F12}~G_{L}U_{F12}~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F12}~C_{M4}U_{F12}~G_{L}U_{F12}~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F12}~C_{M4}U_{F12}~G_{L}U_{F12}~U_{M12}~G_{F}~U_{L12}~C_{F4}~A_{M}~A_{F}~G_{M}
P-A_{M}~C_{F}~A_{L}U_{F18}~U_{M18}A_{L}~A_{M}A_{F}~G_{L}U_{F}~C_{M}U_{F18}~G_{L}U_{F}~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F}~C_{M}U_{F18}~G_{L}U_{F}~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F}~C_{M}U_{F18}~G_{L}U_{F18}~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F}~C_{M}U_{F18}~G_{L}U_{F18}~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F18}~C_{M}U_{F18}~G_{L}U_{F18}~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F18}~C_{M}U_{F18}~G_{L}U_{F18}~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F}~C_{M4}U_{F18}~G_{L}U_{F18}~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F}~C_{M}U_{F18}~G_{L}U_{F18}~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F18}~C_{M4}U_{F18}~G_{L}U_{F18}~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F18}~C_{M4}U_{F18}~G_{L}U_{F18}~U_{M18}~G_{F}~U_{L18}~C_{F4}~A_{M}~A_{F}~G_{M}
P-A_{M}~C_{F}~A_{L}U_{F3}~U_{M3}A_{L}~A_{M}A_{F}~G_{L}U_{F}~C_{M}U_{F3}~G_{L}U~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F}~C_{M}U_{F3}~G_{L}U~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F}~C_{M}U_{F3}~G_{L}U~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F}~C_{M}U_{F3}~G_{L}U~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F3}~C_{M}U_{F3}~G_{L}U~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F3}~C_{M}U_{F3}~G_{L}U~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F}~C_{M4}U_{F3}~G_{L}U~U_{M3}~G_{F}~U_{L3}~C_{F}~A_{M}~A_{F}~G_{M}
P-A_{M}~C_{F4}~A_{M}U_{F3}~U_{M3}A_{L}~A_{M}A_{F}~G_{L}U_{F}~C_{M}U_{F3}~G_{L}U~U_{M3}~G_{F}~U_{L3}~C_{F4}~A_{M}~A_{F}~G_{M}
P-A_{M}~C_{F}~A_{L}U_{F3}~U_{M3}A_{L}~A_{M}A_{F}~G_{L}U_{F3}~C_{M4}U_{F3}~G_{L}U~U_{M3}~G_{F}~U_{L3}~C_{F}~A_{M}~A_{F}~G_{M}
P-A_{M}~C_{F4}~A_{L}U_{F3}~U_{M3}A_{L}~A_{M}A_{F}~G_{L}U_{F3}~C_{M4}U_{F3}~G_{L}U~U_{M3}~G_{F}~U_{L3}~C_{F4}~A_{M}~A_{F}~G_{M}
P-A_{M}~C_{F}~A_{L}U_{F12}~U_{M12}A_{L}~A_{M}A_{F}~G_{L}U_{F}~C_{M}U_{F12}~G_{L}U~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F}~C_{M}U_{F12}~G_{L}U~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F}~C_{M}U_{F12}~G_{L}U~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F}~C_{M}U_{F12}~G_{L}U~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F12}~C_{M}U_{F12}~G_{L}U~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F12}~C_{M}U_{F12}~G_{L}U~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F}~C_{M4}U_{F12}~G_{L}U~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F}~C_{M}U_{F12}~G_{L}U~U_{M12}~G_{F}~U_{L12}~C_{F4}~A_{M}~A_{F}~G_{M}
P-A_{M}~C_{F}~A_{L}U_{F12}~U_{M12}A_{L}~A_{M}A_{F}~G_{L}U_{F12}~C_{M4}U_{F12}~G_{L}U~U_{M12}~G_{F}~U_{L12}~C_{F}~A_{M}~A_{F}~G_{M}
P-A_{M}~C_{F4}~A_{L}U_{F12}~U_{M12}A_{L}~A_{M}A_{F}~G_{L}U_{F12}~C_{M4}U_{F12}~G_{L}U~U_{M12}~G_{F}~U_{L12}~C_{F4}~A_{M}~A_{F}~G_{M}
P-A_{M}~C_{F}~A_{L}U_{F18}~U_{M18}A_{L}~A_{M}A_{F}~G_{L}U_{F}~C_{M}U_{F18}~G_{L}U~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F}~C_{M}U_{F18}~G_{L}U~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F}~C_{M}U_{F18}~G_{L}U~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F}~C_{M}U_{F18}~G_{L}U~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F18}~C_{M}U_{F18}~G_{L}U~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F18}~C_{M}U_{F18}~G_{L}U~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F}~C_{M4}U_{F18}~G_{L}U~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F}~C_{M}U_{F18}~G_{L}U~U_{M18}~G_{F}~U_{L18}~C_{F4}~A_{M}~A_{F}~G_{M}
P-A_{M}~C_{F}~A_{L}U_{F18}~U_{M18}A_{L}~A_{M}A_{F}~G_{L}U_{F18}~C_{M4}U_{F18}~G_{L}U~U_{M18}~G_{F}~U_{L18}~C_{F}~A_{M}~A_{F}~G_{M}
P-A_{M}~C_{F4}~A_{L}U_{F18}~U_{M18}A_{L}~A_{M}A_{F}~G_{L}U_{F18}~C_{M4}U_{F18}~G_{L}U~U_{M18}~G_{F}~U_{L18}~C_{F4}~A_{M}~A_{F}~G_{M}
P-A_{M}~C_{F}~A_{M}U_{F}~U_{M}A_{L}~A_{M}A_{L}~G_{M}U_{F}~C_{M}U_{F}~G_{L}U_{F}~U_{M}~G_{L}~U_{M}~C_{L}~A_{M}~A_{F}~G_{M}
P-A_{M}~C_{F}~A_{M}U_{F}~U_{M}A_{L}~A_{M}A_{F}~G_{L}U_{F}~C_{M}U_{F}~G_{L}U~U_{M}~G_{L}~U_{M}~C_{L}~A_{M}~A_{F}~G_{M}
P-A_{M}~C_{F}~A_{M}U_{F3}~U_{M3}A_{L}~A_{M}A_{L}~G_{M}U_{F}~C_{M}U_{F3}~G_{L}U_{F}~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{L}~G_{M}U_{F}~C_{M}U_{F3}~G_{L}U_{F}~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{L}~G_{M}U_{F}~C_{M}U_{F3}~G_{L}U_{F3}~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{L}~G_{M}U_{F}~C_{M}U_{F3}~G_{L}U_{F3}~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{L}~G_{M}U_{F3}~C_{M}U_{F3}~G_{L}U_{F3}~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{L}~G_{M}U_{F3}~C_{M}U_{F3}~G_{L}U_{F3}~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{L}~G_{M}U_{F}~C_{M4}U_{F3}~G_{L}U_{F3}~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{L}~G_{M}U_{F}~C_{M}U_{F3}~G_{L}U_{F3}~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{L}~G_{M}U_{F3}~C_{M4}U_{F3}~G_{L}U_{F3}~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{L}~G_{M}U_{F3}~C_{M4}U_{F3}~G_{L}U_{F3}~U_{M3}~G_{L}~U_{M3}~C_{L4}~A_{M}~A_{F}~G_{M}
P-A_{M}~C_{F}~A_{M}U_{F12}~U_{M12}A_{L}~A_{M}A_{L}~G_{M}U_{F}~C_{M}U_{F12}~G_{L}U_{F}~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{L}~G_{M}U_{F}~C_{M}U_{F12}~G_{L}U_{F}~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{L}~G_{M}U_{F}~C_{M}U_{F12}~G_{L}U_{F12}~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{L}~G_{M}U_{F}~C_{M}U_{F12}~G_{L}U_{F12}~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{L}~G_{M}U_{F12}~C_{M}U_{F12}~G_{L}U_{F12}~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{L}~G_{M}U_{F12}~C_{M}U_{F12}~G_{L}U_{F12}~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{L}~G_{M}U_{F}~C_{M4}U_{F12}~G_{L}U_{F12}~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{L}~G_{M}U_{F}~C_{M}U_{F12}~G_{L}U_{F12}~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{L}~G_{M}U_{F12}~C_{M4}U_{F12}~G_{L}U_{F12}~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{L}~G_{M}U_{F12}~C_{M4}U_{F12}~G_{L}U_{F12}~U_{M12}~G_{L}~U_{M12}~C_{L4}~A_{M}~A_{F}~G_{M}
P-A_{M}~C_{F}~A_{M}U_{F18}~U_{M18}A_{L}~A_{M}A_{L}~G_{M}U_{F}~C_{M}U_{F18}~G_{L}U_{F}~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{L}~G_{M}U_{F}~C_{M}U_{F18}~G_{L}U_{F}~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{L}~G_{M}U_{F}~C_{M}U_{F18}~G_{L}U_{F18}~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{L}~G_{M}U_{F}~C_{M}U_{F18}~G_{L}U_{F18}~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{L}~G_{M}U_{F18}~C_{M}U_{F18}~G_{L}U_{F18}~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{L}~G_{M}U_{F18}~C_{M}U_{F18}~G_{L}U_{F18}~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{L}~G_{M}U_{F}~C_{M4}U_{F18}~G_{L}U_{F18}~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{L}~G_{M}U_{F}~C_{M}U_{F18}~G_{L}U_{F18}~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{L}~G_{M}U_{F18}~C_{M4}U_{F18}~G_{L}U_{F18}~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{L}~G_{M}U_{F18}~C_{M4}U_{F18}~G_{L}U_{F18}~U_{M18}~G_{L}~U_{M18}~C_{L4}~A_{M}~A_{F}~G_{M}
P-A_{M}~C_{F}~A_{M}U_{F3}~U_{M3}A_{L}~A_{M}A_{F}~G_{L}U_{F}~C_{M}U_{F3}~G_{L}U~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{F}~G_{L}U_{F}~C_{M}U_{F3}~G_{L}U~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{F}~G_{L}U_{F}~C_{M}U_{F3}~G_{L}U~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{F}~G_{L}U_{F}~C_{M}U_{F3}~G_{L}U~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{F}~G_{L}U_{F3}~C_{M}U_{F3}~G_{L}U~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{F}~G_{L}U_{F3}~C_{M}U_{F3}~G_{L}U~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{F}~G_{L}U_{F}~C_{M4}U_{F3}~G_{L}U~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{F}~G_{L}U_{F}~C_{M}U_{F3}~G_{L}U~U_{M3}~G_{L}~U_{M3}~C_{L4}~A_{M}~A_{F}~G_{M}
P-A_{M}~C_{F}~A_{M}U_{F3}~U_{M3}A_{L}~A_{M}A_{F}~G_{L}U_{F3}~C_{M4}U_{F3}~G_{L}U~U_{M3}~G_{L}~U_{M3}~C_{L}~A_{M}~A_{F}~G_{M}
P-A_{M}~C_{F4}~A_{M}U_{F3}~U_{M3}A_{L}~A_{M}A_{F}~G_{L}U_{F3}~C_{M4}U_{F3}~G_{L}U~U_{M3}~G_{L}~U_{M3}~C_{L4}~A_{M}~A_{F}~G_{M}
P-A_{M}~C_{F}~A_{M}U_{F12}~U_{M12}A_{L}~A_{M}A_{F}~G_{L}U_{F}~C_{M}U_{F12}~G_{L}U~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{F}~G_{L}U_{F}~C_{M}U_{F12}~G_{L}U~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{F}~G_{L}U_{F}~C_{M}U_{F12}~G_{L}U~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{F}~G_{L}U_{F}~C_{M}U_{F12}~G_{L}U~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{F}~G_{L}U_{F12}~C_{M}U_{F12}~G_{L}U~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{F}~G_{L}U_{F12}~C_{M}U_{F12}~G_{L}U~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{F}~G_{L}U_{F}~C_{M4}U_{F12}~G_{L}U~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{F}~G_{L}U_{F}~C_{M}U_{F12}~G_{L}U~U_{M12}~G_{L}~U_{M12}~C_{L4}~A_{M}~A_{F}~G_{M}
P-A_{M}~C_{F}~A_{M}U_{F12}~U_{M12}A_{L}~A_{M}A_{F}~G_{L}U_{F12}~C_{M4}U_{F12}~G_{L}U~U_{M12}~G_{L}~U_{M12}~C_{L}~A_{M}~A_{F}~G_{M}
P-A_{M}~C_{F4}~A_{M}U_{F12}~U_{M12}A_{L}~A_{M}A_{F}~G_{L}U_{F12}~C_{M4}U_{F12}~G_{L}U~U_{M12}~G_{L}~U_{M12}~C_{L4}~A_{M}~A_{F}~G_{M}
P-A_{M}~C_{F}~A_{M}U_{F18}~U_{M18}A_{L}~A_{M}A_{F}~G_{L}U_{F}~C_{M}U_{F18}~G_{L}U~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{F}~G_{L}U_{F}~C_{M}U_{F18}~G_{L}U~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{F}~G_{L}U_{F}~C_{M}U_{F18}~G_{L}U~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{F}~G_{L}U_{F}~C_{M}U_{F18}~G_{L}U~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{F}~G_{L}U_{F18}~C_{M}U_{F18}~G_{L}U~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{F}~G_{L}U_{F18}~C_{M}U_{F18}~G_{L}U~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{F}~G_{L}U_{F}~C_{M4}U_{F18}~G_{L}U~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{F}~G_{L}U_{F}~C_{M}U_{F18}~G_{L}U~U_{M18}~G_{L}~U_{M18}~C_{L4}~A_{M}~A_{F}~G_{M}
P-A_{M}~C_{F}~A_{M}U_{F18}~U_{M18}A_{L}~A_{M}A_{F}~G_{L}U_{F18}~C_{M4}U_{F18}~G_{L}U~U_{M18}~G_{L}~U_{M18}~C_{L}~A_{M}~A_{F}~G_{M}
P-A_{M}~C_{F4}~A_{M}U_{F18}~U_{M18}A_{L}~A_{M}A_{F}~G_{L}U_{F18}~C_{M4}U_{F18}~G_{L}U~U_{M18}~G_{L}~U_{M18}~C_{L4}~A_{M}~A_{F}~G_{M}
P-A_{M}~C_{F}~A_{L}U_{F}~U_{M}A_{F}~A_{L}A_{F}~G_{L}U_{F}~C_{M}U_{F}~G_{M}U_{F}~U_{L}~G_{F}~U_{M}~C_{F}~A_{L}~A_{F}~G_{M}
P-A_{M}~C_{F}~A_{L}U_{F}~U_{M}A_{F}~A_{L}A_{F}~G_{L}U_{F}~C_{M}U_{F}~G_{M}U~U_{L}~G_{F}~U_{L}~C_{F}~A_{M}~A_{F}~G_{M}
P-A_{M}~C_{F}~A_{L}U_{F3}~U_{M3}A_{F}~A_{L}A_{F}~G_{L}U_{F}~C_{M}U_{F3}~G_{M}U_{F}~U_{L3}~G_{F}~U_{M3}~C_{F}~A_{L}~A_{F}~G_{M}
P-A_{M}~C_{F4}~A_{L}U_{F3}~U_{M3}A_{F}~A_{L}A_{F}~G_{L}U_{F}~C_{M}U_{F3}~G_{M}U_{F}~U_{L3}~G_{F}~U_{M3}~C_{F4}~A_{L}~A_{F}~G_{M}
P-A_{M}~C_{F}~A_{L}U_{F3}~U_{M3}A_{F}~A_{L}A_{F}~G_{L}U_{F}~C_{M}U_{F3}~G_{M}U_{F3}~U_{L3}~G_{F}~U_{M3}~C_{F}~A_{L}~A_{F}~G_{M}
P-A_{M}~C_{F4}~A_{L}U_{F3}~U_{M3}A_{F}~A_{L}A_{F}~G_{L}U_{F3}~C_{M}U_{F3}~G_{M}U_{F3}~U_{L3}~G_{F}~U_{M3}~C_{F4}~A_{L}~A_{F}~G_{M}
P-A_{M}~C_{F}~A_{L}U_{F3}~U_{M3}A_{F}~A_{L}A_{F}~G_{L}U_{F3}~C_{M}U_{F3}~G_{M}U_{F3}~U_{L3}~G_{F}~U_{M3}~C_{F}~A_{L}~A_{F}~G_{M}
P-A_{M}~C_{F4}~A_{L}U_{F3}~U_{M3}A_{F}~A_{L}A_{F}~G_{L}U_{F3}~C_{M}U_{F3}~G_{M}U_{F3}~U_{L3}~G_{F}~U_{M3}~C_{F4}~A_{L}~A_{F}~G_{M}
P-A_{M}~C_{F}~A_{L}U_{F3}~U_{M3}A_{F}~A_{L}A_{F}~G_{L}U_{F}~C_{M}U_{F3}~G_{M}U_{F3}~U_{L3}~G_{F}~U_{M3}~C_{F}~A_{L}~A_{F}~G_{M}
P-A_{M}~C_{F4}~A_{L}U_{F3}~U_{M3}A_{F}~A_{L}A_{F}~G_{L}U_{F}~C_{M}U_{F3}~G_{M}U_{F3}~U_{L3}~G_{F}~U_{M3}~C_{F4}~A_{L}~A_{F}~G_{M}
P-A_{M}~C_{F}~A_{L}U_{F3}~U_{M3}A_{F}~A_{L}A_{F}~G_{L}U_{F3}~C_{M4}U_{F3}~G_{M}U_{F3}~U_{L3}~G_{F}~U_{M3}~C_{F}~A_{L}~A_{F}~G_{M}
P-A_{M}~C_{F4}~A_{L}U_{F3}~U_{M3}A_{F}~A_{L}A_{F}~G_{L}U_{F3}~C_{M4}U_{F3}~G_{M}U_{F3}~U_{L3}~G_{F}~U_{M3}~C_{F4}~A_{L}~A_{F}~G_{M}
P-A_{M}~C_{F}~A_{L}U_{F12}~U_{M12}A_{F}~A_{L}A_{F}~G_{L}U_{F}~C_{M}U_{F12}~G_{M}U_{F}~U_{L12}~G_{F}~U_{M12}~C_{F}~A_{L}~A_{F}~G_{M}
P-A_{M}~C_{F4}~A_{L}U_{F12}~U_{M12}A_{F}~A_{L}A_{F}~G_{L}U_{F}~C_{M}U_{F12}~G_{M}U_{F}~U_{L12}~G_{F}~U_{M12}~C_{F4}~A_{L}~A_{F}~G_{M}
P-A_{M}~C_{F}~A_{L}U_{F12}~U_{M12}A_{F}~A_{L}A_{F}~G_{L}U_{F}~C_{M}U_{F12}~G_{M}U_{F12}~U_{L12}~G_{F}~U_{M12}~C_{F}~A_{L}~A_{F}~G_{M}
P-A_{M}~C_{F4}~A_{L}U_{F12}~U_{M12}A_{F}~A_{L}A_{F}~G_{L}U_{F}~C_{M}U_{F12}~G_{M}U_{F12}~U_{L12}~G_{F}~U_{M12}~C_{F4}~A_{L}~A_{F}~G_{M}
P-A_{M}~C_{F}~A_{L}U_{F12}~U_{M12}A_{F}~A_{L}A_{F}~G_{L}U_{F12}~C_{M}U_{F12}~G_{M}U_{F12}~U_{L12}~G_{F}~U_{M12}~C_{F}~A_{L}~A_{F}~G_{M}
P-A_{M}~C_{F4}~A_{L}U_{F12}~U_{M12}A_{F}~A_{L}A_{F}~G_{L}U_{F12}~C_{M}U_{F12}~G_{M}U_{F12}~U_{L12}~G_{F}~U_{M12}~C_{F4}~A_{L}~A_{F}~G_{M}
P-A_{M}~C_{F}~A_{L}U_{F12}~U_{M12}A_{F}~A_{L}A_{F}~G_{L}U_{F}~C_{M4}U_{F12}~G_{M}U_{F12}~U_{L12}~G_{F}~U_{M12}~C_{F}~A_{L}~A_{F}~G_{M}
P-A_{M}~C_{F4}~A_{L}U_{F12}~U_{M12}A_{F}~A_{L}A_{F}~G_{L}U_{F}~C_{M}U_{F12}~G_{M}U_{F12}~U_{L12}~G_{F}~U_{M12}~C_{F4}~A_{L}~A_{F}~G_{M}
P-A_{M}~C_{F}~A_{L}U_{F12}~U_{M12}A_{F}~A_{L}A_{F}~G_{L}U_{F12}~C_{M4}U_{F12}~G_{M}U_{F12}~U_{L12}~G_{F}~U_{M12}~C_{F}~A_{L}~A_{F}~G_{M}
P-A_{M}~C_{F4}~A_{L}U_{F12}~U_{M12}A_{F}~A_{L}A_{F}~G_{L}U_{F12}~C_{M4}U_{F12}~G_{M}U_{F12}~U_{L12}~G_{F}~U_{M12}~C_{F4}~A_{L}~A_{F}~G_{M}
P-A_{M}~C_{F}~A_{L}U_{F18}~U_{M18}A_{F}~A_{L}A_{F}~G_{L}U_{F}~C_{M}U_{F18}~G_{M}U_{F}~U_{L18}~G_{F}~U_{M18}~C_{F}~A_{L}~A_{F}~G_{M}
P-A_{M}~C_{F4}~A_{L}U_{F18}~U_{M18}A_{F}~A_{L}A_{F}~G_{L}U_{F}~C_{M}U_{F18}~G_{M}U_{F}~U_{L18}~G_{F}~U_{M18}~C_{F4}~A_{L}~A_{F}~G_{M}
P-A_{M}~C_{F}~A_{L}U_{F18}~U_{M18}A_{F}~A_{L}A_{F}~G_{L}U_{F}~C_{M}U_{F18}~G_{M}U_{F18}~U_{L18}~G_{F}~U_{M18}~C_{F}~A_{L}~A_{F}~G_{M}
P-A_{M}~C_{F4}~A_{L}U_{F18}~U_{M18}A_{F}~A_{L}A_{F}~G_{L}U_{F}~C_{M}U_{F18}~G_{M}U_{F18~U}_{L18}~G_{F}~U_{M18}~C_{F4}~A_{L}~A_{F}~G_{M}
P-A_{M}~C_{F}~A_{L}U_{F18}~U_{M18}A_{F}~A_{L}A_{F}~G_{L}U_{F18}~C_{M}U_{F18}~G_{M}U_{F18}~U_{L18}~G_{F}~U_{M18}~C_{F}~A_{L}~A_{F}~G_{M}
P-A_{M}~C_{F4}~A_{L}U_{F18}~U_{M18}A_{F}~A_{L}A_{F}~G_{L}U_{F18}~C_{M}U_{F18}~G_{M}U_{F18}~U_{L18}~G_{F}~U_{M18}~C_{F4}~A_{L}~A_{F}~G_{M}
P-A_{M}~C_{F}~A_{L}U_{F18}~U_{M18}A_{F}~A_{L}A_{F}~G_{L}U_{F}~C_{M4}U_{F18}~G_{M}U_{F18}~U_{L18}~G_{F}~U_{M18}~C_{F}~A_{L}~A_{F}~G_{M}
P-A_{M}~C_{F4}~A_{L}U_{F18}~U_{M18}A_{F}~A_{L}A_{F}~G_{L}U_{F}~C_{M}U_{F18}~G_{M}U_{F18}U_{L18}~G_{F}~U_{M18}~C_{F4}~A_{L}~A_{F}~G_{M}
P-A_{M}~C_{F}~A_{L}U_{F18}~U_{M18}A_{F}~A_{L}A_{F}~G_{L}U_{F18}~C_{M4}U_{F18}~G_{M}U_{F18}~U_{L18}~G_{F}~U_{M18}~C_{F}~A_{L}~A_{F}~G_{M}
P-A_{M}~C_{F4}~A_{L}U_{F18}~U_{M18}A_{F}~A_{L}A_{F}~G_{L}U_{F18}~C_{M4}U_{F18}~G_{M}U_{F18}~U_{L18}~G_{F}~U_{M18}~C_{F4}~A_{L}~A_{F}~G_{M}
P-A_{M}~C_{F}~A_{L}U_{F3}~U_{M3}A_{F}~A_{L}A_{F}~G_{L}U_{F}~C_{M}U_{F3}~G_{M}U~U_{L3}~G_{F}~U_{L3}~C_{F}~A_{M}~A_{F}~G_{M}
P-A_{M}~C_{F4}~A_{L}U_{F3}~U_{M3}A_{F}~A_{L}A_{F}~G_{L}U_{F}~C_{M}U_{F3}~G_{M}U~U_{L3}~G_{F}~U_{L3}~C_{F4}~A_{M}~A_{F}~G_{M}
P-A_{M}~C_{F}~A_{L}U_{F3}~U_{M3}A_{F}~A_{L}A_{F}~G_{L}U_{F}~C_{M}U_{F3}~G_{M}U~U_{L3}~G_{F}~U_{L3}~C_{F}~A_{M}~A_{F}~G_{M}
P-A_{M}~C_{F4}~A_{L}U_{F3}~U_{M3}A_{F}~A_{L}A_{F}~G_{L}U_{F}~C_{M}U_{F3}~G_{M}U~U_{L3}~G_{F}~U_{L3}~C_{F4}~A_{M}~A_{F}~G_{M}
P-A_{M}~C_{F}~A_{L}U_{F3}~U_{M3}A_{F}~A_{L}A_{F}~G_{L}U_{F3}~C_{M}U_{F3}~G_{M}U~U_{L3}~G_{F}~U_{L3}~C_{F}~A_{M}~A_{F}~G_{M}
P-A_{M}~C_{F4}~A_{L}U_{F3}~U_{M3}A_{F}~A_{L}A_{F}~G_{L}U_{F3}~C_{M}U_{F3}~G_{M}U~U_{L3}~G_{F}~U_{L3}~C_{F4}~A_{M}~A_{F}~G_{M}
P-A_{M}~C_{F}~A_{L}U_{F3}~U_{M3}A_{F}~A_{L}A_{F}~G_{L}U_{F}~C_{M}U_{F3}~G_{M}U~U_{L3}~G_{F}~U_{L3}~C_{F}~A_{M}~A_{F}~G_{M}
P-A_{M}~C_{F4}~A_{L}U_{F3}~U_{M3}A_{F}~A_{L}A_{F}~G_{L}U_{F}~C_{M}U_{F3}~G_{M}U~U_{L3}~G_{F}~U_{L3}~C_{F4}~A_{M}~A_{F}~G_{M}
P-A_{M}~C_{F}~A_{L}U_{F3}~U_{M3}A_{F}~A_{L}A_{F}~G_{L}U_{F3}~C_{M4}U_{F3}~G_{M}U~U_{L3}~G_{F}~U_{L3}~C_{F}~A_{M}~A_{F}~G_{M}
P-A_{M}~C_{F4}~A_{L}U_{F3}~U_{M3}A_{F}~A_{L}A_{F}~G_{L}U_{F3}~C_{M4}U_{F3}~G_{M}U~U_{L3}~G_{F}~U_{L3}~C_{F4}~A_{M}~A_{F}~G_{M}
P-A_{M}~C_{F}~A_{L}U_{F12}~U_{M12}A_{F}~A_{L}A_{F}~G_{L}U_{F}~C_{M}U_{F12}~G_{M}U~U_{L12}~G_{F}~U_{L12}~C_{F}~A_{M}~A_{F}~G_{M}
P-A_{M}~C_{F4}~A_{L}U_{F12}~U_{M12}A_{F}~A_{L}A_{F}~G_{L}U_{F}~C_{M}U_{F12}~G_{M}U~U_{L12}~G_{F}~U_{L12}~C_{F4}~A_{M}~A_{F}~G_{M}
P-A_{M}~C_{F}~A_{L}U_{F12}~U_{M12}A_{F}~A_{L}A_{F}~G_{L}U_{F}~C_{M}U_{F12}~G_{M}U~U_{L12}~G_{F}~U_{L12}~C_{F}~A_{M}~A_{F}~G_{M}
P-A_{M}~C_{F4}~A_{L}U_{F12}~U_{M12}A_{F}~A_{L}A_{F}~G_{L}U_{F}~C_{M}U_{F12}~G_{M}U~U_{L12}~G_{F}~U_{L12}~C_{F4}~A_{M}~A_{F}~G_{M}
P-A_{M}~C_{F}~A_{L}U_{F12}~U_{M12}A_{F}~A_{L}A_{F}~G_{L}U_{F12}~C_{M}U_{F12}~G_{M}U~U_{L12}~G_{F}~U_{L12}~C_{F}~A_{M}~A_{F}~G_{M}
P-A_{M}~C_{F4}~A_{L}U_{F12}~U_{M12}A_{F}~A_{L}A_{F}~G_{L}U_{F12}~C_{M}U_{F12}~G_{M}U~U_{L12}~G_{F}~U_{L12}~C_{F4}~A_{M}~A_{F}~G_{M}
P-A_{M}~C_{F4}~A_{L}U_{F12}~U_{M12}A_{F}~A_{L}A_{F}~G_{L}U_{F}~C_{M4}U_{F12}~G_{M}U~U_{L12}~G_{F}~U_{L12}~C_{F}~A_{M}~A_{F}~G_{M}
P-A_{M}~C_{F4}~A_{L}U_{F12}~U_{M12}A_{F}~A_{L}A_{F}~G_{L}U_{F}~C_{M}U_{F12}~G_{M}U~U_{L12}~G_{F}~U_{L12}~C_{F4}~A_{M}~A_{F}~G_{M}
P-A_{M}~C_{F}~A_{L}U_{F12}~U_{M12}A_{F}~A_{L}A_{F}~G_{L}U_{F12}~C_{M4}U_{F12}~G_{M}U~U_{L12}~G_{F}~U_{L12}~C_{F}~A_{M}~A_{F}~G_{M}
P-A_{M}~C_{F4}~A_{L}U_{F12}~U_{M12}A_{F}~A_{L}A_{F}~G_{L}U_{F12}~C_{M4}U_{F12}~G_{M}U~U_{L12}~G_{F}~U_{L12}~C_{F4}~A_{M}~A_{F}~G_{M}
P-A_{M}~C_{F}~A_{L}U_{F18}~U_{M18}A_{F}~A_{L}A_{F}~G_{L}U_{F}~C_{M}U_{F18}~G_{M}U~U_{L18}~G_{F}~U_{L18}~C_{F}~A_{M}~A_{F}~G_{M}
P-A_{M}~C_{F4}~A_{L}U_{F18}~U_{M18}A_{F}~A_{L}A_{F}~G_{L}U_{F}~C_{M}U_{F18}~G_{M}U~U_{L18}~G_{F}~U_{L18}~C_{F4}~A_{M}~A_{F}~G_{M}
P-A_{M}~C_{F}~A_{L}U_{F18}~U_{M18}A_{F}~A_{L}A_{F}~G_{L}U_{F}~C_{M}U_{F18}~G_{M}U~U_{L18}~G_{F}~U_{L18}~C_{F}~A_{M}~A_{F}~G_{M}
P-A_{M}~C_{F4}~A_{L}U_{F18}~U_{M18}A_{F}~A_{L}A_{F}~G_{L}U_{F}~C_{M}U_{F18}~G_{M}U~U_{L18}~G_{F}~U_{L18}~C_{F4}~A_{M}~A_{F}~G_{M}
P-A_{M}~C_{F}~A_{L}U_{F18}~U_{M18}A_{F}~A_{L}A_{F}~G_{L}U_{F18}~C_{M}U_{F18}~G_{M}U~U_{L18}~G_{F}~U_{L18}~C_{F}~A_{M}~A_{F}~G_{M}
P-A_{M}~C_{F4}~A_{L}U_{F18}~U_{M18}A_{F}~A_{L}A_{F}~G_{L}U_{F18}~C_{M}U_{F18}~G_{M}U~U_{L18}~G_{F}~U_{L18}~C_{F4}~A_{M}~A_{F}~G_{M}
P-A_{M}~C_{F}~A_{L}U_{F18}~U_{M18}A_{F}~A_{L}A_{F}~G_{L}U_{F}~C_{M4}U_{F18}~G_{M}U~U_{L18}~G_{F}~U_{L18}~C_{F}~A_{M}~A_{F}~G_{M}
P-A_{M}~C_{F4}~A_{L}U_{F18}~U_{M18}A_{F}~A_{L}A_{F}~G_{L}U_{F}~C_{M}U_{F18}~G_{M}U~U_{L18}~G_{F}~U_{L18}~C_{F4}~A_{M}~A_{F}~G_{M}
P-A_{M}~C_{F}~A_{L}U_{F18}~U_{M18}A_{F}~A_{L}A_{F}~G_{L}U_{F18}~C_{M4}U_{F18}~G_{M}U~U_{L18}~G_{F}~U_{L18}~C_{F}~A_{M}~A_{F}~G_{M}
P-A_{M}~C_{F4}~A_{L}U_{F18}~U_{M18}A_{F}~A_{L}A_{F}~G_{L}U_{F18}~C_{M4}U_{F18}~G_{M}U~U_{L18}~G_{F}~U_{L18}~C_{F}~A_{M}~A_{F}~G_{M}
P-A_{M}~C_{F}~A_{M}U_{L}~U_{M}A_{F}~A_{M}A_{L}~G_{M}U_{F}~C_{M}U_{L}~G_{M}U_{F}~U_{L}~G_{F}~U_{M}~C_{L}~A_{M}~A_{F}~G_{M}
P-A_{M}~C_{F}~A_{M}U_{L}~U_{M}A_{F}~A_{M}A_{L}~G_{M}U_{F}~C_{M}U_{L}~G_{M}U~U_{L}~G_{F}~U_{L}~C_{F}~A_{M}~A_{F}~G_{M}
P-A_{M}~C_{F}~A_{M}U_{L3}~U_{M3}A_{F}~A_{M}A_{L}~G_{M}U_{F}~C_{M}U_{L3}~G_{M}U_{F}~U_{L3}~G_{F}~U_{M3}~C_{L}~A_{M}~A_{F}~G_{M}
P-A_{M}~C_{F4}~A_{M}U_{L3}~U_{M3}A_{F}~A_{M}A_{L}~G_{M}U_{F}~C_{M}U_{L3}~G_{M}U_{F}~U_{L3}~G_{F}~U_{M3}~C_{L4}~A_{M}~A_{F}~G_{M}
P-A_{M}~C_{F}~A_{M}U_{L3}~U_{M3}A_{F}~A_{M}A_{L}~G_{M}U_{F}~C_{M}U_{L3}~G_{M}U_{F3}~U_{L3}~G_{F}~U_{M3}~C_{L}~A_{M}~A_{F}~G_{M}
P-A_{M}~C_{F4}~A_{M}U_{L3}~U_{M3}A_{F}~A_{M}A_{L}~G_{M}U_{F}~C_{M}U_{L3}~G_{M}U_{F3}~U_{L3}~G_{F}~U_{M3}~C_{L4}~A_{M}~A_{F}~G_{M}
P-A_{M}~C_{F}~A_{M}U_{L3}~U_{M3}A_{F}~A_{M}A_{L}~G_{M}U_{F3}~C_{M}U_{L3}~G_{M}U_{F3}~U_{L3}~G_{F}~U_{M3}~C_{L}~A_{M}~A_{F}~G_{M}
P-A_{M}~C_{F4}~A_{M}U_{L3}~U_{M3}A_{F}~A_{M}A_{L}~G_{M}U_{F3}~C_{M}U_{L3}~G_{M}U_{F3}~U_{L3}~G_{F}~U_{M3}~C_{L4}~A_{M}~A_{F}~G_{M}
P-A_{M}~C_{F}~A_{M}U_{L3}~U_{M3}A_{F}~A_{M}A_{L}~G_{M}U_{F}~C_{M4}U_{L3}~G_{M}U_{F3}~U_{L3}~G_{F}~U_{M3}~C_{L}~A_{M}~A_{F}~G_{M}
P-A_{M}~C_{F4}~A_{M}U_{L3}~U_{M3}A_{F}~A_{M}A_{L}~G_{M}U_{F}~C_{M}U_{L3}~G_{M}U_{F3}~U_{L3}~G_{F}~U_{M3}~C_{L4}~A_{M}~A_{F}~G_{M}
P-A_{M}~C_{F}~A_{M}U_{L3}~U_{M3}A_{F}~A_{M}A_{L}~G_{M}U_{F3}~C_{M4}U_{L3}~G_{M}U_{F3}~U_{L3}~G_{F}~U_{M3}~C_{L}~A_{M}~A_{F}~G_{M}
P-A_{M}~C_{F4}~A_{M}U_{L3}~U_{M3}A_{F}~A_{M}A_{L}~G_{M}U_{F3}~C_{M4}U_{L3}~G_{M}U_{F3}~U_{L3}~G_{F}~U_{M3}~C_{L4}~A_{M}~A_{F}~G_{M}
P-A_{M}~C_{F}~A_{M}U_{L12}~U_{M12}A_{F}~A_{M}A_{L}~G_{M}U_{F}~C_{M}U_{L12}~G_{M}U_{F}~U_{L12}~G_{F}~U_{M12}~C_{L}~A_{M}~A_{F}~G_{M}
P-A_{M}~C_{F4}~A_{M}U_{L12}~U_{M12}A_{F}~A_{M}A_{L}~G_{M}U_{F}~C_{M}U_{L12}~G_{M}U_{F}~U_{L12}~G_{F}~U_{M12}~C_{L4}~A_{M}~A_{F}~G_{M}
P-A_{M}~C_{F}~A_{M}U_{L12}~U_{M12}A_{F}~A_{M}A_{L}~G_{M}U_{F}~C_{M}U_{L12}~G_{M}U_{F}~U_{L12}~G_{F}~U_{M12}~C_{L}~A_{M}~A_{F}~G_{M}
P-A_{M}~C_{F4}~A_{M}U_{L12}~U_{M12}A_{F}~A_{M}A_{L}~G_{M}U_{F}~C_{M}U_{L12}~G_{M}U_{F}~U_{L12}~G_{F}~U_{M12}~C_{L4}~A_{M}~A_{F}~G_{M}
P-A_{M}~C_{F}~A_{M}U_{L12}~U_{M12}A_{F}~A_{M}A_{L}~G_{M}U_{F12}~C_{M}U_{L12}~G_{M}U_{F12}~U_{L12}~G_{F}~U_{M12}~C_{L}~A_{M}~A_{F}~G_{M}
P-A_{M}~C_{F4}~A_{M}U_{L12}~U_{M12}A_{F}~A_{M}A_{L}~G_{M}U_{F12}~C_{M}U_{L12}~G_{M}U_{F12}~U_{L12}~G_{F}~U_{M12}~C_{L4}~A_{M}~A_{F}~G_{M}
P-A_{M}~C_{F}~A_{M}U_{L12}~U_{M12}A_{F}~A_{M}A_{L}~G_{M}U_{F}~C_{M4}U_{L12}~G_{M}U_{F12}~U_{L12}~G_{F}~U_{M12}~C_{L}~A_{M}~A_{F}~G_{M}
P-A_{M}~C_{F4}~A_{M}U_{L12}~U_{M12}A_{F}~A_{M}A_{L}~G_{M}U_{F}~C_{M}U_{L12}~G_{M}U_{F12}~U_{L12}~G_{F}~U_{M12}~C_{L4}~A_{M}~A_{F}~G_{M}
P-A_{M}~C_{F}~A_{M}U_{L12}~U_{M12}A_{F}~A_{M}A_{L}~G_{M}U_{F12}~C_{M4}U_{L12}~G_{M}U_{F12}~U_{L12}~G_{F}~U_{M12}~C_{L}~A_{M}~A_{F}~G_{M}
P-A_{M}~C_{F4}~A_{M}U_{L12}~U_{M12}A_{F}~A_{M}A_{L}~G_{M}U_{F12}~C_{M4}U_{L12}~G_{M}U_{F12}~U_{L12}~G_{F}~U_{M12}~C_{L4}~A_{M}~A_{F}~G_{M}
P-A_{M}~C_{F}~A_{M}U_{L18}~U_{M18}A_{F}~A_{M}A_{L}~G_{M}U_{F}~C_{M}U_{L18}~G_{M}U_{F}~U_{L18}~G_{F}~U_{M18}~C_{L}~A_{M}~A_{F}~G_{M}
P-A_{M}~C_{F4}~A_{M}U_{L18}~U_{M18}A_{F}~A_{M}A_{L}~G_{M}U_{F}~C_{M}U_{L18}~G_{M}U_{F}~U_{L18}~G_{F}~U_{M18}~C_{L4}~A_{M}~A_{F}~G_{M}
P-A_{M}~C_{F}~A_{M}U_{L18}~U_{M18}A_{F}~A_{M}A_{L}~G_{M}U_{F}~C_{M}U_{L18}~G_{M}U_{F18}~U_{L18}~G_{F}~U_{M18}~C_{L}~A_{M}~A_{F}~G_{M}
P-A_{M}~C_{F4}~A_{M}U_{L18}~U_{M18}A_{F}~A_{M}A_{L}~G_{M}U_{F}~C_{M}U_{L18}~G_{M}U_{F18}~U_{L18}~G_{F}~U_{M18}~C_{L4}~A_{M}~A_{F}~G_{M}
P-A_{M}~C_{F}~A_{M}U_{L18}~U_{M18}A_{F}~A_{M}A_{L}~G_{M}U_{F18}~C_{M}U_{L18}~G_{M}U_{F18}~U_{L18}~G_{F}~U_{M18}~C_{L}~A_{M}~A_{F}~G_{M}
P-A_{M}~C_{F4}~A_{M}U_{L18}~U_{M18}A_{F}~A_{M}A_{L}~G_{M}U_{F18}~C_{M}U_{L18}~G_{M}U_{F18}~U_{L18}~G_{F}~U_{M18}~C_{L4}~A_{M}~A_{F}~G_{M}
P-A_{M}~C_{F}~A_{M}U_{L18}~U_{M18}A_{F}~A_{M}A_{L}~G_{M}U_{F}~C_{M4}U_{L18}~G_{M}U_{F18}~U_{L18}~G_{F}~U_{M18}~C_{L}~A_{M}~A_{F}~G_{M}
P-A_{M}~C_{F4}~A_{M}U_{L18}~U_{M18}A_{F}~A_{M}A_{L}~G_{M}U_{F}~C_{M}U_{L18}~G_{M}U_{F18}~U_{L18}~G_{F}~U_{M18}~C_{L4}~A_{M}~A_{F}~G_{M}
P-A_{M}~C_{F}~A_{M}U_{L18}~U_{M18}A_{F}~A_{M}A_{L}~G_{M}U_{F18}~C_{M4}U_{L18}~G_{M}U_{F18}~U_{L18}~G_{F}~U_{M18}~C_{L}~A_{M}~A_{F}~G_{M}
P-A_{M}~C_{F4}~A_{M}U_{L18}~U_{M18}A_{F}~A_{M}A_{L}~G_{M}U_{F18}~C_{M4}U_{L18}~G_{M}U_{F18}~U_{L18}~G_{F}~U_{M18}~C_{L4}~A_{M}~A_{F}~G_{M}
P-A_{M}~C_{F}~A_{M}U_{L3}~U_{M3}A_{F}~A_{M}A_{L}~G_{M}U_{F}~C_{M}U_{L3}~G_{M}U~U_{L3}~G_{F}~U_{L3}~C_{F}~A_{M}~A_{F}~G_{M}
P-A_{M}~C_{F4}~A_{M}U_{L3}~U_{M3}A_{F}~A_{M}A_{L}~G_{M}U_{F}~C_{M}U_{L3}~G_{M}U~U_{L3}~G_{F}~U_{L3}~C_{F}~A_{M}~A_{F}~G_{M}
P-A_{M}~C_{F}~A_{M}U_{L3}~U_{M3}A_{F}~A_{M}A_{L}~G_{M}U_{F}~C_{M}U_{L3}~G_{M}U~U_{L3}~G_{F}~U_{L3}~C_{F}~A_{M}~A_{F}~G_{M}
P-A_{M}~C_{F4}~A_{M}U_{L3}~U_{M3}A_{F}~A_{M}A_{L}~G_{M}U_{F}~C_{M}U_{L3}~G_{M}U~U_{L3}~G_{F}~U_{L3}~C_{F4}~A_{M}~A_{F}~G_{M}
P-A_{M}~C_{F}~A_{M}U_{L3}~U_{M3}A_{F}~A_{M}A_{L}~G_{M}U_{F3}~C_{M}U_{L3}~G_{M}U~U_{L3}~G_{F}~U_{L3}~C_{F}~A_{M}~A_{F}~G_{M}
P-A_{M}~C_{F4}~A_{M}U_{L3}~U_{M3}A_{F}~A_{M}A_{L}~G_{M}U_{F3}~C_{M}U_{L3}~G_{M}U~U_{L3}~G_{F}~U_{L3}~C_{F4}~A_{M}~A_{F}~G_{M}
P-A_{M}~C_{F}~A_{M}U_{L3}~U_{M3}A_{F}~A_{M}A_{L}~G_{M}U_{F}~C_{M4}U_{L3}~G_{M}U~U_{L3}~G_{F}~U_{L3}~C_{F}~A_{M}~A_{F}~G_{M}
P-A_{M}~C_{F4}~A_{M}U_{L3}~U_{M3}A_{F}~A_{M}A_{L}~G_{M}U_{F}~C_{M}U_{L3}~G_{M}U~U_{L3}~G_{F}~U_{L3}~C_{F4}~A_{M}~A_{F}~G_{M}
P-A_{M}~C_{F}~A_{M}U_{L3}~U_{M3}A_{F}~A_{M}A_{L}~G_{M}U_{F3}~C_{M4}U_{L3}~G_{M}U~U_{L3}~G_{F}~U_{L3}~C_{F}~A_{M}~A_{F}~G_{M}
P-A_{M}~C_{F4}~A_{M}U_{L3}~U_{M3}A_{F}~A_{M}A_{L}~G_{M}U_{F3}~C_{M4}U_{L3}~G_{M}U~U_{L3}~G_{F}~U_{L3}~C_{F}~A_{M}~A_{F}~G_{M}
P-A_{M}~C_{F}~A_{M}U_{L12}~U_{M12}A_{F}~A_{M}A_{L}~G_{M}U_{F}~C_{M}U_{L12}~G_{M}U~U_{L12}~G_{F}~U_{L12}~C_{F}~A_{M}~A_{F}~G_{M}
P-A_{M}~C_{F4}~A_{M}U_{L12}~U_{M12}A_{F}~A_{M}A_{L}~G_{M}U_{F}~C_{M}U_{L12}~G_{M}U~U_{L12}~G_{F}~U_{L12}~C_{F4}~A_{M}~A_{F}~G_{M}
P-A_{M}~C_{F}~A_{M}U_{L12}~U_{M12}A_{F}~A_{M}A_{L}~G_{M}U_{F}~C_{M}U_{L12}~G_{M}U~U_{L12}~G_{F}~U_{L12}~C_{F}~A_{M}~A_{F}~G_{M}
P-A_{M}~C_{F4}~A_{M}U_{L12}~U_{M12}A_{F}~A_{M}A_{L}~G_{M}U_{F}~C_{M}U_{L12}~G_{M}U~U_{L12}~G_{F}~U_{L12}~C_{F4}~A_{M}~A_{F}~G_{M}
P-A_{M}~C_{F}~A_{M}U_{L12}~U_{M12}A_{F}~A_{M}A_{L}~G_{M}U_{F12}~C_{M}U_{L12}~G_{M}U~U_{L12}~G_{F}~U_{L12}~C_{F}~A_{M}~A_{F}~G_{M}
P-A_{M}~C_{F4}~A_{M}U_{L12}~U_{M12}A_{F}~A_{M}A_{L}~G_{M}U_{F12}~C_{M}U_{L12}~G_{M}U~U_{L12}~G_{F}~U_{L12}~C_{F4}~A_{M}~A_{F}~G_{M}
P-A_{M}~C_{F}~A_{M}U_{L12}~U_{M12}A_{F}~A_{M}A_{L}~G_{M}U_{F}~C_{M4}U_{L12}~G_{M}U~U_{L12}~G_{F}~U_{L12}~C_{F}~A_{M}~A_{F}~G_{M}
P-A_{M}~C_{F4}~A_{M}U_{L12}~U_{M12}A_{F}~A_{M}A_{L}~G_{M}U_{F}~C_{M}U_{L12}~G_{M}U~U_{L12}~G_{F}~U_{L12}~C_{F4}~A_{M}~A_{F}~G_{M}
P-A_{M}~C_{F}~A_{M}U_{L12}~U_{M12}A_{F}~A_{M}A_{L}~G_{M}U_{F12}~C_{M4}U_{L12}~G_{M}U~U_{L12}~G_{F}~U_{L12}~C_{F}~A_{M}~A_{F}~G_{M}
P-A_{M}~C_{F4}~A_{M}U_{L12}~U_{M12}A_{F}~A_{M}A_{L}~G_{M}U_{F12}~C_{M4}U_{L12}~G_{M}U~U_{L12}~G_{F}~U_{L12}~C_{F4}~A_{M}~A_{F}~G_{M}
P-A_{M}~C_{F}~A_{M}U_{L18}~U_{M18}A_{F}~A_{M}A_{L}~G_{M}U_{F}~C_{M}U_{L18}~G_{M}U~U_{L18}~G_{F}~U_{L18}~C_{F}~A_{M}~A_{F}~G_{M}
P-A_{M}~C_{F4}~A_{M}U_{L18}~U_{M18}A_{F}~A_{M}A_{L}~G_{M}U_{F}~C_{M}U_{L18}~G_{M}U~U_{L18}~G_{F}~U_{L18}~C_{F4}~A_{M}~A_{F}~G_{M}
P-A_{M}~C_{F}~A_{M}U_{L18}~U_{M18}A_{F}~A_{M}A_{L}~G_{M}U_{F}~C_{M}U_{L18}~G_{M}U~U_{L18}~G_{F}~U_{L18}~C_{F}~A_{M}~A_{F}~G_{M}
P-A_{M}~C_{F4}~A_{M}U_{L18}~U_{M18}A_{F}~A_{M}A_{L}~G_{M}U_{F}~C_{M}U_{L18}~G_{M}U~U_{L18}~G_{F}~U_{L18}~C_{F4}~A_{M}~A_{F}~G_{M}
P-A_{M}~C_{F}~A_{M}U_{L18}~U_{M18}A_{F}~A_{M}A_{L}~G_{M}U_{F18}~C_{M}U_{L18}~G_{M}U~U_{L18}~G_{F}~U_{L18}~C_{F}~A_{M}~A_{F}~G_{M}
P-A_{M}~C_{F4}~A_{M}U_{L18}~U_{M18}A_{F}~A_{M}A_{L}~G_{M}U_{F18}~C_{M}U_{L18}~G_{M}U~U_{L18}~G_{F}~U_{L18}~C_{F4}~A_{M}~A_{F}~G_{M}
P-A_{M}~C_{F}~A_{M}U_{L18}~U_{M18}A_{F}~A_{M}A_{L}~G_{M}U_{F}~C_{M4}U_{L18}~G_{M}U~U_{L18}~G_{F}~U_{L18}~C_{F}~A_{M}~A_{F}~G_{M}
P-A_{M}~C_{F4}~A_{M}U_{L18}~U_{M18}A_{F}~A_{M}A_{L}~G_{M}U_{F}~C_{M}U_{L18}~G_{M}U~U_{L18}~G_{F}~U_{L18}~C_{F4}~A_{M}~A_{F}~G_{M}
P-A_{M}~C_{F}~A_{M}U_{L18}~U_{M18}A_{F}~A_{M}A_{L}~G_{M}U_{F18}~C_{M4}U_{L18}~G_{M}U~U_{L18}~G_{F}~U_{L18}~C_{F}~A_{M}~A_{F}~G_{M}
P-A_{M}~C_{F4}~A_{M}U_{L18}~U_{M18}A_{F}~A_{M}A_{L}~G_{M}U_{F18}~C_{M4}U_{L18}~G_{M}U~U_{L18}~G_{F}~U_{L18}~C_{F4}~A_{M}~A_{F}~G_{M}

### Table 14

### Examples of Murine SSB ags-siRNA Compositions

### A. Murine SSB Antisense Sequence:

UUUUAUUUCACCAUGAUUU

Haringsma et al. (Nucleic Acids Res 40: 4125-36, 2012) used this sequence to generate ssRNAi compounds against murine SSB target site 963 including the following:

P-U_{F}U_{F}U_{F}U_{F}A_{F}U_{F}U_{F}U_{F}C_{F}A_{F}C_{F}C_{F}A_{F}U_{F}G_{F}A_{F}U_{F}U_{F}U_{F}U_{M}U_{M}

Any of the 5′-end nucleosides (P-U_{F} or P-U_{M}) shown in the examples of agsRNAi compounds in Tables 14A and 14B can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Tables 14A and 14B with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the compounds in Tables 14A and 14B can any of those units and linkages provided for herein for overhang precursors.

B. Examples of Application of AGSD to this Compound:

TABLE 14A
Compositions of Matter Suitable for Use in vitro or in a
Subject with a Protective Carrier (5′-3′)

P-U_{F}U_{F}U_{F}U_{F3}A_{F}U_{F3}U_{F}U_{F}C_{F4}A_{F}C_{F}C_{F4}A_{F}U_{F}G_{F}A_{F}U_{F3}U_{F}U_{F}U_{M}U_{M}
P-U_{F}U_{F}U_{F3}U_{F3}A_{F}U_{F3}U_{F3}U_{F}C_{F4}A_{F}C_{F}C_{F}A_{F}U_{F}G_{F}A_{F}U_{F3}U_{F3}U_{F}U_{M}U_{M}
P-U_{F}U_{F3}U_{F3}U_{F3}A_{F}U_{F3}U_{F3}U_{F3}C_{F}A_{F}C_{F}C_{F}A_{F}U_{F}G_{F}A_{F}U_{F3}U_{F3}U_{F3}U_{M}U_{M}
P-U_{F}U_{F3}U_{F3}U_{F3}A_{F}U_{F3}U_{F3}U_{F3}C_{F4}A_{F}C_{F4}A_{F}U_{F}G_{F}A_{F}U_{F3}U_{F3}U_{F3}U_{M}U_{M}
P-U_{F}U_{F}U_{F}U_{F12}A_{F}U_{F12}U_{F}U_{F}C_{F4}A_{F}C_{F}C_{F4}A_{F}U_{F}G_{F}A_{F}U_{F12}U_{F}U_{F}U_{M}U_{M}
P-U_{F}U_{F}U_{F12}U_{F12}A_{F}U_{F12}U_{F12}U_{F}C_{F4}A_{F}C_{F}C_{F}A_{F}U_{F}G_{F}A_{F}U_{F12}U_{F12}U_{F}U_{M}U_{M}
P-U_{F}U_{F12}U_{F12}U_{F12}A_{F}U_{F12}U_{F12}U_{F12}C_{F}A_{F}C_{F}C_{F}A_{F}U_{F}G_{F}A_{F}U_{F12}U_{F12}U_{F12}U_{M}U_{M}
P-U_{F}U_{F12}U_{F12}U_{F12}A_{F}U_{F12}U_{F12}U_{F12}C_{F4}A_{F}C_{F}C_{F4}A_{F}U_{F}G_{F}A_{F}U_{F12}U_{F12}U_{F12}U_{M}U_{M}
P-U_{F}U_{F}U_{F}U_{F18}A_{F}U_{F18}U_{F}U_{F}C_{F4}A_{F}C_{F}C_{F4}A_{F}U_{F}G_{F}A_{F}U_{F18}U_{F}U_{F}U_{M}U_{M}
P-U_{F}U_{F}U_{F18}U_{F18}A_{F}U_{F18}U_{F18}U_{F}C_{F4}A_{F}C_{F}C_{F}A_{F}U_{F}G_{F}A_{F}U_{F18}U_{F18}U_{F}U_{M}U_{M}
P-U_{F}U_{F18}U_{F18}U_{F18}A_{F}U_{F18}U_{F18}U_{F18}C_{F}A_{F}C_{F}C_{F}A_{F}U_{F}G_{F}A_{F}U_{F18}U_{F18}U_{F18}U_{M}U_{M}
P-U_{F}U_{F18}U_{F18}U_{F18}A_{F}U_{F18}U_{F18}U_{F18}C_{F4}A_{F}C_{F}C_{F4}A_{F}U_{F}G_{F}A_{F}U_{F18}U_{F18}U_{F18}U_{M}U_{M}
P-U_{F}U_{F}U_{F}U_{F3}A_{F}U_{F3}U_{F}U_{F}C_{F4}A_{F}C_{F}C_{F4}A_{F}UG_{F}A_{F}U_{F3}U_{F}U_{F}U_{M}U_{M}
P-U_{F}U_{F}U_{F3}U_{F3}A_{F}U_{F3}U_{F3}U_{F}C_{F4}A_{F}C_{F}C_{F}A_{F}UG_{F}A_{F}U_{F3}U_{F3}U_{F}U_{M}U_{M}
P-U_{F}U_{F3}U_{F3}U_{F3}A_{F}U_{F3}U_{F3}U_{F3}C_{F}A_{F}C_{F}C_{F}A_{F}UG_{F}A_{F}U_{F3}U_{F3}U_{F3}U_{M}U_{M}
P-U_{F}U_{F3}U_{F3}U_{F}3A_{F}U_{F3}U_{F3}U_{F3}C_{F4}A_{F}C_{F}C_{F4}A_{F}UG_{F}A_{F}U_{F3}U_{F3}U_{F3}U_{M}U_{M}
P-U_{F}U_{F}U_{F}U_{F12}A_{F}U_{F12}U_{F}U_{F}C_{F4}A_{F}C_{F}C_{F4}A_{F}UG_{F}A_{F}U_{F12}U_{F}U_{F}U_{M}U_{M}
P-U_{F}U_{F}U_{F12}U_{F12}A_{F}U_{F12}U_{F12}U_{F}C_{F4}A_{F}C_{F}C_{F}A_{F}UG_{F}A_{F}U_{F12}U_{F12}U_{F}U_{M}U_{M}
P-U_{F}U_{F12}U_{F12}U_{F12}A_{F}U_{F12}U_{F12}U_{F12}C_{F}A_{F}C_{F}C_{F}A_{F}UG_{F}A_{F}U_{F12}U_{F12}U_{F12}U_{M}U_{M}
P-U_{F}U_{F12}U_{F12}U_{F12}A_{F}U_{F12}U_{F12}U_{F12}C_{F4}A_{F}C_{F}C_{F4}A_{F}UG_{F}A_{F}U_{F12}U_{F12}U_{F12}U_{M}U_{M}
P-U_{F}U_{F}U_{F}U_{F18}A_{F}U_{F18}U_{F}U_{F}C_{F4}A_{F}C_{F}C_{F4}A_{F}UG_{F}A_{F}U_{F18}U_{F}U_{F}U_{M}U_{M}
P-U_{F}U_{F}U_{F18}U_{F18}A_{F}U_{F18}U_{F18}U_{F}C_{F4}A_{F}C_{F}C_{F}A_{F}UG_{F}A_{F}U_{F18}U_{F18}U_{F}U_{M}U_{M}
P-U_{F}U_{F18}U_{F18}U_{F18}A_{F}U_{F18}U_{F18}U_{F18}C_{F}A_{F}C_{F}C_{F}A_{F}UG_{F}A_{F}U_{F18}U_{F18}U_{F18}U_{M}U_{M}
P-U_{F}U_{F18}U_{F18}U_{F18}A_{F}U_{F18}U_{F18}U_{F18}C_{F4}A_{F}C_{F}C_{F4}A_{F}UG_{F}A_{F}U_{F18}U_{F18}U_{F18}U_{M}U_{M}
P-U_{F}U_{F}U_{L}U_{F}A_{L}U_{F}U_{F}U_{F}C_{L}A_{F}C_{F}C_{F}A_{F}U_{F}G_{L}A_{F}U_{L}U_{F}U_{F}U_{M}U_{M}
P-U_{F}U_{F}U_{L}U_{F3}A_{L}U_{F3}U_{F}U_{F}C_{L4}A_{F}C_{F}C_{F4}A_{F}U_{F}G_{L}A_{F}U_{L3}U_{F}U_{F}U_{M}U_{M}
P-U_{F}U_{F}U_{L3}U_{F3}A_{L}U_{F3}U_{F3}U_{F}C_{L4}A_{F}C_{F}C_{F}A_{F}U_{F}G_{L}A_{F}U_{L3}U_{F3}U_{F}U_{M}U_{M}
P-U_{F}U_{F3}U_{L3}U_{F3}A_{L}U_{F3}U_{F3}U_{F3}C_{L}A_{F}C_{F}C_{F}A_{F}U_{F}G_{L}A_{F}U_{L3}U_{F3}U_{F3}U_{M}U_{M}
P-U_{F}U_{F3}U_{L3}U_{F3}A_{L}U_{F3}U_{F3}U_{F3}C_{L4}A_{F}C_{F}C_{F4}A_{F}U_{F}G_{L}A_{F}U_{L3}U_{F3}U_{F3}U_{M}U_{M}
P-U_{F}U_{F}U_{L}U_{F12}A_{L}U_{F12}U_{F}U_{F}C_{L4}A_{F}C_{F}C_{F4}A_{F}U_{F}G_{L}A_{F}U_{L12}U_{F}U_{F}U_{M}U_{M}
P-U_{F}U_{F}U_{L12}U_{F12}A_{L}U_{F12}U_{F12}U_{F}C_{L4}A_{F}C_{F}C_{F}A_{F}U_{F}G_{L}A_{F}U_{L12}U_{F12}U_{F}U_{M}U_{M}
P-U_{F}U_{F12}U_{L12}U_{F12}A_{L}U_{F12}U_{F12}U_{F12}C_{L}A_{F}C_{F}C_{F}A_{F}U_{F}G_{L}A_{F}U_{L12}U_{F12}U_{F12}U_{M}U_{M}
P-U_{F}U_{F12}U_{L12}U_{F12}A_{L}U_{F12}U_{F12}U_{F12}C_{L4}A_{F}C_{F}C_{F4}A_{F}U_{F}G_{L}A_{F}U_{L12}U_{F12}U_{F12}U_{M}U_{M}
P-U_{F}U_{F}U_{L}U_{F18}A_{L}U_{F18}U_{F}U_{F}C_{L4}A_{F}C_{F}C_{F4}A_{F}U_{F}G_{L}A_{F}U_{L18}U_{F}U_{F}U_{M}U_{M}
P-U_{F}U_{F}U_{L18}U_{F18}A_{L}U_{F18}U_{F18}U_{F}C_{L4}A_{F}C_{F}C_{F}A_{F}U_{F}G_{L}A_{F}U_{L18}U_{F18}U_{F}U_{M}U_{M}
P-U_{F}U_{F18}U_{L18}U_{F18}A_{L}U_{F18}U_{F18}U_{F18}C_{L}A_{F}C_{F}C_{F}A_{F}U_{F}G_{L}A_{F}U_{L18}U_{F18}U_{F18}U_{M}U_{M}
P-U_{F}U_{F18}U_{L18}U_{F18}A_{L}U_{F18}U_{F18}U_{F18}C_{L4}A_{F}C_{F}C_{F4}A_{F}U_{F}G_{L}A_{F}U_{L18}U_{F18}U_{F18}U_{M}U_{M}
P-U_{F}U_{F}U_{L}U_{F3}A_{L}U_{F3}U_{F}U_{F}C_{L4}A_{F}C_{F}C_{F4}A_{F}UG_{L}A_{F}U_{L3}U_{F}U_{F}U_{M}U_{M}
P-U_{F}U_{F}U_{L3}U_{F3}A_{L}U_{F3}U_{F3}U_{F}C_{L4}A_{F}C_{F}C_{F}A_{F}UG_{L}A_{F}U_{L3}U_{F3}U_{F}U_{M}U_{M}
P-U_{F}U_{F3}U_{L3}U_{F3}A_{L}U_{F3}U_{F3}U_{F3}C_{L}A_{F}C_{F}C_{F}A_{F}UG_{L}A_{F}U_{L3}U_{F3}U_{F3}U_{M}U_{M}
P-U_{F}U_{F3}U_{L3}U_{F3}A_{L}U_{F3}U_{F3}U_{F3}C_{L4}A_{F}C_{F}C_{F4}A_{F}UG_{L}A_{F}U_{L3}U_{F3}U_{F3}U_{M}U_{M}
P-U_{F}U_{F}U_{L}U_{F12}A_{L}U_{F12}U_{F}U_{F}C_{L4}A_{F}C_{F}C_{F4}A_{F}UG_{L}A_{F}U_{L12}U_{F}U_{F}U_{M}U_{M}
P-U_{F}U_{F}U_{L12}U_{F12}A_{L}U_{F12}U_{F12}U_{F}C_{L4}A_{F}C_{F}C_{F}A_{F}UG_{L}A_{F}U_{L12}U_{F12}U_{F}U_{M}U_{M}
P-U_{F}U_{F12}U_{L12}U_{F12}A_{L}U_{F12}U_{F12}U_{F12}C_{L}A_{F}C_{F}C_{F}A_{F}UG_{L}A_{F}U_{L12}U_{F12}U_{F12}U_{M}U_{M}
P-U_{F}U_{F12}U_{L12}U_{F12}A_{L}U_{F12}U_{F12}U_{F12}C_{L4}A_{F}C_{F}C_{F4}A_{F}UG_{L}A_{F}U_{L12}U_{F12}U_{F12}U_{M}U_{M}
P-U_{F}U_{F}U_{L}U_{F18}A_{L}U_{F18}U_{F}U_{F}C_{L4}A_{F}C_{F}C_{F4}A_{F}UG_{L}A_{F}U_{L18}U_{F}U_{F}U_{M}U_{M}
P-U_{F}U_{F}U_{L18}U_{F18}A_{L}U_{F18}U_{F18}U_{F}C_{L4}A_{F}C_{F}C_{F}A_{F}UG_{L}A_{F}U_{L18}U_{F18}U_{F}U_{M}U_{M}
P-U_{F}U_{F18}U_{L18}U_{F18}A_{L}U_{F18}U_{F18}U_{F18}C_{L}A_{F}C_{F}C_{F}A_{F}UG_{L}A_{F}U_{L18}U_{F18}U_{F18}U_{M}U_{M}
P-U_{F}U_{F18}U_{L18}U_{F18}A_{L}U_{F18}U_{F18}U_{F18}C_{L4}A_{F}C_{F}C_{F4}A_{F}UG_{L}A_{F}U_{L18}U_{F18}U_{F18}U_{M}U_{M}
P-U_{F}U_{F}U_{L}U_{F}A_{F}U_{L}U_{F}U_{F}C_{F}A_{F}C_{F}C_{F}A_{L}U_{F}G_{L}A_{F}U_{L}U_{F}U_{F}U_{M}U_{M}
P-U_{F}U_{F}U_{L}U_{F3}A_{F}U_{L3}U_{F}U_{F}C_{F4}A_{F}C_{F}C_{F4}A_{L}U_{F}G_{L}A_{F}U_{L3}U_{F}U_{F}U_{M}U_{M}
P-U_{F}U_{F}U_{L3}U_{F3}A_{F}U_{L3}U_{F3}U_{F}C_{F4}A_{F}C_{F}C_{F}A_{L}U_{F}G_{L}A_{F}U_{L3}U_{F3}U_{F}U_{M}U_{M}
P-U_{F}U_{F3}U_{L3}U_{F3}A_{F}U_{L3}U_{F3}U_{F3}C_{F}A_{F}C_{F}C_{F}A_{L}U_{F}G_{L}A_{F}U_{L3}U_{F3}U_{F3}U_{M}U_{M}
P-U_{F}U_{F3}U_{L3}U_{F3}A_{F}U_{L3}U_{F3}U_{F3}C_{F4}A_{F}C_{F}C_{F4}A_{L}U_{F}G_{L}A_{F}U_{L3}U_{F3}U_{F3}U_{M}U_{M}
P-U_{F}U_{F}U_{L}U_{F12}A_{F}U_{L12}U_{F}U_{F}C_{F4}A_{F}C_{F}C_{F4}A_{L}U_{F}G_{L}A_{F}U_{L12}U_{F}U_{F}U_{M}U_{M}
P-U_{F}U_{F}U_{L12}U_{F12}A_{F}U_{L12}U_{F12}U_{F}C_{F4}A_{F}C_{F}C_{F}A_{L}U_{F}G_{L}A_{F}U_{L12}U_{F12}U_{F}U_{M}U_{M}
P-U_{F}U_{F12}U_{L12}U_{F12}A_{F}U_{L12}U_{F12}U_{F12}C_{F}A_{F}C_{F}C_{F}A_{L}U_{F}G_{L}A_{F}U_{L12}U_{F12}U_{F12}U_{M}U_{M}
P-U_{F}U_{F12}U_{L12}U_{F12}A_{F}U_{L12}U_{F12}U_{F12}C_{F4}A_{F}C_{F}C_{F4}A_{L}U_{F}G_{L}A_{F}U_{L12}U_{F12}U_{F12}U_{M}U_{M}
P-U_{F}U_{F}U_{L}U_{F18}A_{F}U_{L18}U_{F}U_{F}C_{F4}A_{F}C_{F}C_{F4}A_{L}U_{F}G_{L}A_{F}U_{L18}U_{F}U_{F}U_{M}U_{M}
P-U_{F}U_{F}U_{L18}U_{F18}A_{F}U_{L18}U_{F18}U_{F}C_{F4}A_{F}C_{F}C_{F}A_{L}U_{F}G_{L}A_{F}U_{L18}U_{F18}U_{F}U_{M}U_{M}
P-U_{F}U_{F18}U_{L18}U_{F18}A_{F}U_{L18}U_{F18}U_{F18}C_{F}A_{F}C_{F}C_{F}A_{L}U_{F}G_{L}A_{F}U_{L18}U_{F18}U_{F18}U_{M}U_{M}
P-U_{F}U_{F18}U_{L18}U_{F18}A_{F}U_{L18}U_{F18}U_{F18}C_{F4}A_{F}C_{F}C_{F4}A_{L}U_{F}G_{L}A_{F}U_{L18}U_{F18}U_{F18}U_{M}U_{M}
P-U_{F}U_{F}U_{L}U_{F3}A_{F}U_{L3}U_{F}U_{F}C_{F4}A_{F}C_{F}C_{F4}A_{L}UG_{L}A_{F}U_{L3}U_{F}U_{F}U_{M}U_{M}
P-U_{F}U_{F}U_{L3}U_{F3}A_{F}U_{L3}U_{F3}U_{F}C_{F4}A_{F}C_{F}C_{F}A_{L}UG_{L}A_{F}U_{L3}U_{F3}U_{F}U_{M}U_{M}
P-U_{F}U_{F3}U_{L3}U_{F3}A_{F}U_{L3}U_{F3}U_{F3}C_{F}A_{F}C_{F}C_{F}A_{L}UG_{L}A_{F}U_{L3}U_{F3}U_{F3}U_{M}U_{M}
P-U_{F}U_{F3}U_{L3}U_{F3}A_{F}U_{L3}U_{F3}U_{F3}C_{F4}A_{F}C_{F}C_{F4}A_{L}UG_{L}A_{F}U_{L3}U_{F3}U_{F3}U_{M}U_{M}
P-U_{F}U_{F}U_{L}U_{F12}A_{F}U_{L12}U_{F}U_{F}C_{F4}A_{F}C_{F}C_{F4}A_{L}UG_{L}A_{F}U_{L12}U_{F}U_{F}U_{M}U_{M}
P-U_{F}U_{F}U_{L12}U_{F12}A_{F}U_{L12}U_{F12}U_{F}C_{F4}A_{F}C_{F}C_{F}A_{L}UG_{L}A_{F}U_{L12}U_{F12}U_{F}U_{M}U_{M}
P-U_{F}U_{F12}U_{L12}U_{F12}A_{F}U_{L12}U_{F12}U_{F12}C_{F}A_{F}C_{F}C_{F}A_{L}UG_{L}A_{F}U_{L12}U_{F12}U_{F12}U_{M}U_{M}
P-U_{F}U_{F12}U_{L12}U_{F12}A_{F}U_{L12}U_{F12}U_{F12}C_{F4}A_{F}C_{F}C_{F4}A_{L}UG_{L}A_{F}U_{L12}U_{F12}U_{F12}U_{M}U_{M}
P-U_{F}U_{F}U_{L}U_{F18}A_{F}U_{L18}U_{F}U_{F}C_{F4}A_{F}C_{F}C_{F4}A_{L}UG_{L}A_{F}U_{L18}U_{F}U_{F}U_{M}U_{M}
P-U_{F}U_{F}U_{L18}U_{F18}A_{F}U_{L18}U_{F18}U_{F}C_{F4}A_{F}C_{F}C_{F}A_{L}UG_{L}A_{F}U_{L18}U_{F18}U_{F}U_{M}U_{M}
P-U_{F}U_{F18}U_{L18}U_{F18}A_{F}U_{L18}U_{F18}U_{F18}C_{F}A_{F}C_{F}C_{F}A_{L}UG_{L}A_{F}U_{L18}U_{F18}U_{F18}U_{M}U_{M}
P-U_{F}U_{F18}U_{L18}U_{F18}A_{F}U_{L18}U_{F18}U_{F18}C_{F4}A_{F}C_{F}C_{F4}A_{L}UG_{L}A_{F}U_{L18}U_{F18}U_{F18}U_{M}U_{M}
P-U_{F}U_{F}U_{F}U_{F}A_{L}U_{F}U_{F}U_{L}C_{F}A_{F}C_{F}C_{L}A_{F}U_{F}G_{F}A_{F}U_{L}U_{F}U_{L}U_{M}U_{M}
P-U_{F}U_{F}U_{F}U_{F3}A_{L}U_{F3}U_{F}U_{L}C_{F4}A_{F}C_{F}C_{L4}A_{F}U_{F}G_{F}A_{F}U_{L3}U_{F}U_{L}U_{M}U_{M}
P-U_{F}U_{F}U_{F3}U_{F3}A_{L}U_{F3}U_{F3}U_{L}C_{F4}A_{F}C_{F}C_{L}A_{F}U_{F}G_{F}A_{F}U_{L3}U_{F3}U_{L}U_{M}U_{M}
P-U_{F}U_{F3}U_{F3}U_{F3}A_{L}U_{F3}U_{F3}U_{L3}C_{F}A_{F}C_{F}C_{L}A_{F}U_{F}G_{F}A_{F}U_{L3}U_{F3}U_{L3}U_{M}U_{M}
P-U_{F}U_{F3}U_{F3}U_{F3}A_{L}U_{F3}U_{F3}U_{L3}C_{F4}A_{F}C_{F}C_{L4}A_{F}U_{F}G_{F}A_{F}U_{L3}U_{F3}U_{L3}U_{M}U_{M}
P-U_{F}U_{F}U_{F}U_{F12}A_{L}U_{F12}U_{F}U_{L}C_{F4}A_{F}C_{F}C_{L4}A_{F}U_{F}G_{F}A_{F}U_{L12}U_{F}U_{L}U_{M}U_{M}
P-U_{F}U_{F}U_{F12}U_{F12}A_{L}U_{F12}U_{F12}U_{L}C_{F4}A_{F}C_{F}C_{L}A_{F}U_{F}G_{F}A_{F}U_{L12}U_{F12}U_{L}U_{M}U_{M}
P-U_{F}U_{F12}U_{F12}U_{F12}A_{L}U_{F12}U_{F12}U_{L12}C_{F}A_{F}C_{F}C_{L}A_{F}U_{F}G_{F}A_{F}U_{L12}U_{F12}U_{L12}U_{M}U_{M}
P-U_{F}U_{F12}U_{F12}U_{F12}A_{L}U_{F12}U_{F12}U_{L12}C_{F4}A_{F}C_{F}C_{L4}A_{F}U_{F}G_{F}A_{F}U_{L12}U_{F12}U_{L12}U_{M}U_{M}
P-U_{F}U_{F}U_{F}U_{F18}A_{L}U_{F18}U_{F}U_{L}C_{F4}A_{F}C_{F}C_{L4}A_{F}U_{F}G_{F}A_{F}U_{L18}U_{F}U_{L}U_{M}U_{M}
P-U_{F}U_{F}U_{F18}U_{F18}A_{L}U_{F18}U_{F18}U_{L}C_{F4}A_{F}C_{F}C_{L}A_{F}U_{F}G_{F}A_{F}U_{L18}U_{F18}U_{L}U_{M}U_{M}
P-U_{F}U_{F18}U_{F18}U_{F18}A_{L}U_{F18}U_{F18}U_{L18}C_{F}A_{F}C_{F}C_{L}A_{F}U_{F}G_{F}A_{F}U_{L18}U_{F18}U_{L18}U_{M}U_{M}
P-U_{F}U_{F18}U_{F18}U_{F18}A_{L}U_{F18}U_{F18}U_{L18}C_{F4}A_{F}C_{F}C_{L4}A_{F}U_{F}G_{F}A_{F}U_{L18}U_{F18}U_{L18}U_{M}U_{M}
P-U_{F}U_{F}U_{F}U_{F3}A_{L}U_{F3}U_{F}U_{L}C_{F4}A_{F}C_{F}C_{L4}A_{F}UG_{F}A_{F}U_{L3}U_{F}U_{L}U_{M}U_{M}
P-U_{F}U_{F}U_{F3}U_{F3}A_{L}U_{F3}U_{F3}U_{L}C_{F4}A_{F}C_{F}C_{L}A_{F}UG_{F}A_{F}U_{L3}U_{F3}U_{L}U_{M}U_{M}
P-U_{F}U_{F3}U_{F3}U_{F3}A_{L}U_{F3}U_{F3}U_{L3}C_{F}A_{F}C_{F}C_{L}A_{F}UG_{F}A_{F}U_{L3}U_{F3}U_{L3}U_{M}U_{M}
P-U_{F}U_{F3}U_{F3}U_{F3}A_{L}U_{F3}U_{F3}U_{L3}C_{F4}A_{F}C_{F}C_{L4}A_{F}UG_{F}A_{F}U_{L3}U_{F3}U_{L3}U_{M}U_{M}
P-U_{F}U_{F}U_{F}U_{F12}A_{L}U_{F12}U_{F}U_{L}C_{F4}A_{F}C_{F}C_{L4}A_{F}UG_{F}A_{F}U_{L12}U_{F}U_{L}U_{M}U_{M}
P-U_{F}U_{F}U_{F12}U_{F12}A_{L}U_{F12}U_{F12}U_{L}C_{F4}A_{F}C_{F}C_{L}A_{F}UG_{F}A_{F}U_{L12}U_{F12}U_{L}U_{M}U_{M}
P-U_{F}U_{F12}U_{F12}U_{F12}A_{L}U_{F12}U_{F12}U_{L12}C_{F}A_{F}C_{F}C_{L}A_{F}UG_{F}A_{F}U_{L12}U_{F12}U_{L12}U_{M}U_{M}
P-U_{F}U_{F12}U_{F12}U_{F12}A_{L}U_{F12}U_{F12}U_{L12}C_{F4}A_{F}C_{F}C_{L4}A_{F}UG_{F}A_{F}U_{L12}U_{F12}U_{L12}U_{M}U_{M}
P-U_{F}U_{F}U_{F}U_{F18}A_{L}U_{F18}U_{F}U_{L}C_{F4}A_{F}C_{F}C_{L4}A_{F}UG_{F}A_{F}U_{L18}U_{F}U_{L}U_{M}U_{M}
P-U_{F}U_{F}U_{F18}U_{F18}A_{L}U_{F18}U_{F18}U_{L}C_{F4}A_{F}C_{F}C_{L}A_{F}UG_{F}A_{F}U_{L18}U_{F18}U_{L}U_{M}U_{M}
P-U_{F}U_{F18}U_{F18}U_{F18}A_{L}U_{F18}U_{F18}U_{L18}C_{F}A_{F}C_{F}C_{L}A_{F}UG_{F}A_{F}U_{L18}U_{F18}U_{L18}U_{M}U_{M}
P-U_{F}U_{F18}U_{F18}U_{F18}A_{L}U_{F18}U_{F18}U_{L18}C_{F4}A_{F}C_{F}C_{L4}A_{F}UG_{F}A_{F}U_{L18}U_{F18}U_{L18}U_{M}U_{M}

TABLE 14B
Compositions of Matter Suitable for Administration to a Subject
with or without a Protective Carrier and for Any Other use
as Provided for Herein (5′-3′)

P-U_{M}~U_{F}~U_{M}U_{F3}~A_{M}U_{F3}~U_{M}U_{F}~C_{M4}A_{F}~C_{M}C_{F4}~A_{M}U_{F}~G_{M}~A_{F}~U_{M3}~U_{F}~U_{M}~A_{F}~G_{M}
P-U_{M}~U_{F}~U_{M3}U_{F3}~A_{M}U_{F3}~U_{M3}U_{F}~C_{M4}A_{F}~C_{M}C_{F}~A_{M}U_{F}~G_{M}~A_{F}~U_{M3}~U_{F3}~U_{M}~A_{F}~G_{M}
P-U_{M}~U_{F3}~U_{M3}U_{F3}~A_{M}U_{F3}~U_{M3}U_{F3}~C_{M}A_{F}~C_{M}C_{F}~A_{M}U_{F}~G_{M}~A_{F}~U_{M3}~U_{F3}~U_{M3}~A_{F}~G_{M}
P-U_{M}~U_{F3}~U_{M3}U_{F3}~A_{M}U_{F3}~U_{M3}U_{F3}~C_{M3}A_{F}~C_{M}C_{F3}~A_{M}U_{F}~G_{M}~A_{F}~U_{M3}~U_{F3}~U_{M3}~A_{F}~G_{M}
P-U_{M}~U_{F}~U_{M}U_{F12}~A_{M}U_{F12}~U_{M}U_{F}~C_{M4}A_{F}~C_{M}C_{F4}~A_{M}U_{F}~G_{M}~A_{F}~U_{M12}~U_{F}~U_{M}~A_{F}~G_{M}
P-U_{M}~U_{F}~U_{M12}U_{F12}~A_{M}U_{F12}~U_{M12}U_{F}~C_{M4}A_{F}~C_{M}C_{F4}~A_{M}U_{F}~G_{M}~A_{F}~U_{M12}~U_{F12}~U_{M}~A_{F}~G_{M}
P-U_{M}~U_{F12}~U_{M12}U_{F12}~A_{M}U_{F12}~U_{M12}U_{F12}~C_{M}A_{F}~C_{M}C_{F4}~A_{M}U_{F}~G_{M}~A_{F}~U_{M12}~U_{F12}~U_{M12}~A_{F}~G_{M}
P-U_{M}~U_{F12}~U_{M12}U_{F12}~A_{M}U_{F12}~U_{M12}U_{F12}~C_{M12}A_{F}~C_{M}C_{F12}~A_{M}U_{F}~G_{M}~A_{F}~U_{M12}~U_{F12}~U_{M12}~A_{F}~G_{M}
P-U_{M}~U_{F}~U_{M}U_{F12}~A_{M}U_{F18}~U_{M}U_{F}~C_{M4}A_{F}~C_{M}C_{F4}~A_{M}U_{F}~G_{M}~A_{F}~U_{M18}~U_{F}~U_{M}~A_{F}~G_{M}
P-U_{M}~U_{F}~U_{M18}U_{F18}~A_{M}U_{F18}~U_{M18}U_{F}~C_{M4}A_{F}~C_{M}C_{F}~A_{M}U_{F}~G_{M}~A_{F}~U_{M18}~U_{F18}~U_{M}~A_{F}~G_{M}
P-U_{M}~U_{F18}~U_{M18}U_{F18}~A_{M}U_{F18}~U_{M18}U_{F18}~C_{M}A_{F}~C_{M}C_{F}~A_{M}U_{F}~G_{M}~A_{F}~U_{M18}~U_{F18}~U_{M18}~A_{F}~G_{M}
P-U_{M}~U_{F18}~U_{M18}U_{F18}~A_{M}U_{F18}~U_{M18}U_{F18}~C_{M18}A_{F}~C_{M}C_{F18}~A_{M}U_{F}~G_{M}~A_{F}~U_{M18}~U_{F18}~U_{M18}~A_{F}~G_{M}
P-U_{M}~U_{F}~U_{M}U_{F3}~A_{M}U_{F3}~U_{M}U_{F}~C_{M4}A_{F}~C_{M}C_{F4}~A_{M}U~G_{M}~A_{F}~U_{M3}~U_{F}~U_{M}~A_{F}~G_{M}
P-U_{M}~U_{F}~U_{M3}U_{F3}~A_{M}U_{F3}~U_{M3}U_{F}~C_{M4}A_{F}~C_{M}C_{F}~A_{M}U~G_{M}~A_{F}~U_{M3}~U_{F3}~U_{M}~A_{F}~G_{M}
P-U_{M}~U_{F3}~U_{M3}U_{F3}~A_{M}U_{F3}~U_{M3}U_{F3}~C_{M}A_{F}~C_{M}C_{F}~A_{M}U~G_{M}~A_{F}~U_{M3}~U_{F3}~U_{M3}~A_{F}~G_{M}
P-U_{M}~U_{F3}~U_{M3}U_{F3}~A_{M}U_{F3}~U_{M3}U_{F3}~C_{M3}A_{F}~C_{M}C_{F3}~A_{M}U~G_{M}~A_{F}~U_{M3}~U_{F3}~U_{M3}~A_{F}~G_{M}
P-U_{M}~U_{F}~U_{M}U_{F12}~A_{M}U_{F12}~U_{M}U_{F}~C_{M4}A_{F}~C_{M}C_{F4}~A_{M}U~G_{M}~A_{F}~U_{M12}~U_{F}~U_{M}~A_{F}~G_{M}
P-U_{M}~U_{F}~U_{M12}U_{F12}~A_{M}U_{F12}~U_{M12}U_{F}~C_{M4}A_{F}~C_{M}C_{F4}~A_{M}U~G_{M}~A_{F}~U_{M12}~U_{F12}~U_{M}~A_{F}~G_{M}
P-U_{M}~U_{F12}~U_{M12}U_{F12}~A_{M}U_{F12}~U_{M12}U_{F12}~C_{M}A_{F}~C_{M}C_{F4}~A_{M}U~G_{M}~A_{F}~U_{M12}~U_{F12}~U_{M12}~A_{F}~G_{M}
P-U_{M}~U_{F12}~U_{M12}U_{F12}~A_{M}U_{F12}~U_{M12}U_{F12}~C_{M12}A_{F}~C_{M}C_{F12}~A_{M}U~G_{M}~A_{F}~U_{M12}~U_{F12}~U_{M12}~A_{F}~G_{M}
P-U_{M}~U_{F}~U_{M}U_{F12}~A_{M}U_{F18}~U_{M}U_{F}~C_{M4}A_{F}~C_{M}C_{F4}~A_{M}U~G_{M}~A_{F}~U_{M18}~U_{F}~U_{M}~A_{F}~G_{M}
P-U_{M}~U_{F}~U_{M18}U_{F18}~A_{M}U_{F18}~U_{M18}U_{F}~C_{M4}A_{F}~C_{M}C_{F}~A_{M}U~G_{M}~A_{F}~U_{M18}~U_{F18}~U_{M}~A_{F}~G_{M}
P-U_{M}~U_{F18}~U_{M18}U_{F18}~A_{M}U_{F18}~U_{M18}U_{F18}~C_{M}A_{F}~C_{M}C_{F}~A_{M}U~G_{M}~A_{F}~U_{M18}~U_{F18}~U_{M18}~A_{F}~G_{M}
P-U_{M}~U_{F18}~U_{M18}U_{F18}~A_{M}U_{F18}~U_{M18}U_{F18}~C_{M18}A_{F}~C_{M}C_{F18}~A_{M}U~G_{M}~A_{F}~U_{M18}~U_{F18}~U_{M18}~A_{F}~G_{M}
P-U_{M}~U_{F}~U_{L}U_{F}~A_{L}U_{F}~U_{M}U_{F}~C_{L}A_{F}~C_{M}C_{F}~A_{M}U_{F}~G_{L}~A_{F}~U_{L}~U_{F}~U_{M}~A_{F}~G_{M}
P-U_{M}~U_{F}~U_{L}U_{F3}~A_{L}U_{F3}~U_{M}U_{F}~C_{L4}A_{F}~C_{M}C_{F4}~A_{M}U_{F}~G_{L}~A_{F}~U_{L3}~U_{F}~U_{M}~A_{F}~G_{M}
P-U_{M}~U_{F}~U_{L3}U_{F3}~A_{L}U_{F3}~U_{M3}U_{F}~C_{L4}A_{F}~C_{M}C_{F}~A_{M}U_{F}~G_{L}~A_{F}~U_{L3}~U_{F3}~U_{M}~A_{F}~G_{M}
P-U_{M}~U_{F3}~U_{L3}U_{F3}~A_{L}U_{F3}~U_{M3}U_{F3}~C_{L}A_{F}~C_{M}C_{F}~A_{M}U_{F}~G_{L}~A_{F}~U_{L3}~U_{F3}~U_{M3}~A_{F}~G_{M}
P-U_{M}~U_{F3}~U_{L3}U_{F3}~A_{L}U_{F3}~U_{M3}U_{F3}~C_{L3}A_{F}~C_{M}C_{F3}~A_{M}U_{F}~G_{L}~A_{F}~U_{L3}~U_{F3}~U_{M3}~A_{F}~G_{M}
P-U_{M}~U_{F}~U_{L}U_{F12}~A_{L}U_{F12}~U_{M}U_{F}~C_{L4}A_{F}~C_{M}C_{F4}~A_{M}U_{F}~G_{L}~A_{F}~U_{L12}~U_{F}~U_{M}~A_{F}~G_{M}
P-U_{M}~U_{F}~U_{L12}U_{F12}~A_{L}U_{F12}~U_{M12}U_{F}~C_{L4}A_{F}~C_{M}C_{F}~A_{M}U_{F}~G_{L}~A_{F}~U_{L12}~U_{F12}~U_{M}~A_{F}~G_{M}
P-U_{M}~U_{F12}~U_{L12}U_{F12}~A_{L}U_{F12}~U_{M12}U_{F12}~C_{L}A_{F}~C_{M}C_{F}~A_{M}U_{F}~G_{L}~A_{F}~U_{L12}~U_{F12}~U_{M12}~A_{F}~G_{M}
P-U_{M}~U_{F12}~U_{L12}U_{F12}~A_{L}U_{F12}~U_{M12}U_{F12}~C_{L12}A_{F}~C_{M}C_{F12}~A_{M}U_{F}~G_{L}~A_{F}~U_{L12}~U_{F12}~U_{M12}~A_{F}~G_{M}
P-U_{M}~U_{F}~U_{L}U_{F18}~A_{L}U_{F18}~U_{M}U_{F}~C_{L4}A_{F}~C_{M}C_{F4}~A_{M}U_{F}~G_{L}~A_{F}~U_{L18}~U_{F}~U_{M}~A_{F}~G_{M}
P-U_{M}~U_{F}~U_{L18}U_{F18}~A_{L}U_{F18}~U_{M18}U_{F}~C_{L4}A_{F}~C_{M}C_{F}~A_{M}U_{F}~G_{L}~A_{F}~U_{L18}~U_{F18}~U_{M}~A_{F}~G_{M}
P-U_{M}~U_{F18}~U_{L18}U_{F18}~A_{L}U_{F18}~U_{M18}U_{F18}~C_{L}A_{F}~C_{M}C_{F}~A_{M}U_{F}~G_{L}~A_{F}~U_{L18}~U_{F18}~U_{M18}~A_{F}~G_{M}
P-U_{M}~U_{F18}~U_{L18}U_{F18}~A_{L}U_{F18}~U_{M18}U_{F18}~C_{L18}A_{F}~C_{M}C_{F18}~A_{M}U_{F}~G_{L}~A_{F}~U_{L18}~U_{F18}~U_{M18}~A_{F}~G_{M}
P-U_{M}~U_{F}~U_{L}U_{F3}~A_{L}U_{F3}~U_{M}U_{F}~C_{L4}A_{F}~C_{M}C_{F4}~A_{M}U~G_{L}~A_{F}~U_{L3}~U_{F}~U_{M}~A_{F}~G_{M}
P-U_{M}~U_{F}~U_{L3}U_{F3}~A_{L}U_{F3}~U_{M3}U_{F}~C_{L4}A_{F}~C_{M}C_{F}~A_{M}U~G_{L}~A_{F}~U_{L3}~U_{F3}~U_{M}~A_{F}~G_{M}
P-U_{M}~U_{F3}~U_{L3}U_{F3}~A_{L}U_{F3}~U_{M3}U_{F3}~C_{L}A_{F}~C_{M}C_{F}~A_{M}U~G_{L}~A_{F}~U_{L3}~U_{F3}~U_{M3}~A_{F}~G_{M}
P-U_{M}~U_{F3}~U_{L3}U_{F3}~A_{L}U_{F3}~U_{M3}U_{F3}~C_{L3}A_{F}~C_{M}C_{F3}~A_{M}U~G_{L}~A_{F}~U_{L3}~U_{F3}~U_{M3}~A_{F}~G_{M}
P-U_{M}~U_{F}~U_{L}U_{F12}~A_{L}U_{F12}~U_{M}U_{F}~C_{L4}A_{F}~C_{M}C_{F4}~A_{M}U~G_{L}~A_{F}~U_{L12}~U_{F}~U_{M}~A_{F}~G_{M}
P-U_{M}~U_{F}~U_{L12}U_{F12}~A_{L}U_{F12}~U_{M12}U_{F}~C_{L4}A_{F}~C_{M}C_{F}~A_{M}U~G_{L}~A_{F}~U_{L12}~U_{F12}~U_{M}~A_{F}~G_{M}
P-U_{M}~U_{F12}~U_{L12}U_{F12}~A_{L}U_{F12}~U_{M12}U_{F12}~C_{L}A_{F}~C_{M}C_{F}~A_{M}U~G_{L}~A_{F}~U_{L12}~U_{F12}~U_{M12}~A_{F}~G_{M}
P-U_{M}~U_{F12}~U_{L12}U_{F12}~A_{L}U_{F12}~U_{M12}U_{F12}~C_{L12}A_{F}~C_{M}C_{F12}~A_{M}U~G_{L}~A_{F}~U_{L12}~U_{F12}~U_{M12}~A_{F}~G_{M}
P-U_{M}~U_{F}~U_{L}U_{F18}~A_{L}U_{F18}~U_{M}U_{F}~C_{L4}A_{F}~C_{M}C_{F4}~A_{M}U~G_{L}~A_{F}~U_{L18}~U_{F}~U_{M}~A_{F}~G_{M}
P-U_{M}~U_{F}~U_{L18}U_{F18}~A_{L}U_{F18}~U_{M18}U_{F}~C_{L4}A_{F}~C_{M}C_{F}~A_{M}U~G_{L}~A_{F}~U_{L18}~U_{F18}~U_{M}~A_{F}~G_{M}
P-U_{M}~U_{F18}~U_{L18}U_{F18}~A_{L}U_{F18}~U_{M18}U_{F18}~C_{L}A_{F}~C_{M}C_{F}~A_{M}U~G_{L}~A_{F}~U_{L18}~U_{F18}~U_{M18}~A_{F}~G_{M}
P-U_{M}~U_{F18}~U_{L18}U_{F18}~A_{L}U_{F18}~U_{M18}U_{F18}~C_{L18}A_{F}~C_{M}C_{F18}~A_{M}U~G_{L}~A_{F}~U_{L18}~U_{F18}~U_{M18}~A_{F}~G_{M}
P-U_{M}~U_{F}~U_{L}U_{F}~A_{M}U_{L}~U_{M}U_{F}~C_{M}A_{F}~C_{M}C_{F}~A_{L}U_{F}~G_{L}~A_{F}~U_{L}~U_{F}~U_{M}~A_{F}~G_{M}
P-U_{M}~U_{F}~U_{L}U_{F3}~A_{M}U_{L3}~U_{M}U_{F}~C_{M4}A_{F}~C_{M}C_{F4}~A_{L}U_{F}~G_{L}~A_{F}~U_{L3}~U_{F}~U_{M}~A_{F}~G_{M}
P-U_{M}~U_{F}~U_{L3}U_{F3}~A_{M}U_{L3}~U_{M3}U_{F}~C_{M4}A_{F}~C_{M}C_{F}~A_{L}U_{F}~G_{L}~A_{F}~U_{L3}~U_{F3}~U_{M}~A_{F}~G_{M}
P-U_{M}~U_{F3}~U_{L3}U_{F3}~A_{M}U_{L3}~U_{M3}U_{F3}~C_{M}A_{F}~C_{M}C_{F}~A_{L}U_{F}~G_{L}~A_{F}~U_{L3}~U_{F3}~U_{M3}~A_{F}~G_{M}
P-U_{M}~U_{F3}~U_{L3}U_{F3}~A_{M}U_{L3}~U_{M3}U_{F3}~C_{M3}A_{F}~C_{M}C_{F3}~A_{L}U_{F}~G_{L}~A_{F}~U_{L3}~U_{F3}~U_{M3}~A_{F}~G_{M}
P-U_{M}~U_{F}~U_{L}U_{F12}~A_{M}U_{L12}~U_{M}U_{F}~C_{M4}A_{F}~C_{M}C_{F4}~A_{L}U_{F}~G_{L}~A_{F}~U_{L12}~U_{F}~U_{M}~A_{F}~G_{M}
P-U_{M}~U_{F}~U_{L12}U_{F12}~A_{M}U_{L12}~U_{M12}U_{F}~C_{M4}A_{F}~C_{M}C_{F}~A_{L}U_{F}~G_{L}~A_{F}~U_{L12}~U_{F12}~U_{M}~A_{F}~G_{M}
P-U_{M}~U_{F12}~U_{L12}U_{F12}~A_{M}U_{L12}~U_{M12}U_{F12}~C_{M}A_{F}~C_{M}C_{F}~A_{L}U_{F}~G_{L}~A_{F}~U_{L12}~U_{F12}~U_{M12}~A_{F}~G_{M}
P-U_{M}~U_{F12}~U_{L12}U_{F12}~A_{M}U_{L12}~U_{M12}U_{F12}~C_{M12}A_{F}~C_{M}C_{F12}~A_{L}U_{F}~G_{L}~A_{F}~U_{L12}~U_{F12}~U_{M12}~A_{F}~G_{M}
P-U_{M}~U_{F}~U_{L}U_{F18}~A_{M}U_{L18}~U_{M}U_{F}~C_{M4}A_{F}~C_{M}C_{F4}~A_{L}U_{F}~G_{L}~A_{F}~U_{L18}~U_{F}~U_{M}~A_{F}~G_{M}
P-U_{M}~U_{F}~U_{L18}U_{F18}~A_{M}U_{L18}~U_{M18}U_{F}~C_{M4}A_{F}~C_{M}C_{F}~A_{L}U_{F}~G_{L}~A_{F}~U_{L18}~U_{F18}~U_{M}~A_{F}~G_{M}
P-U_{M}~U_{F18}~U_{L18}U_{F18}~A_{M}U_{L18}~U_{M18}U_{F18}~C_{M}A_{F}~C_{M}C_{F}~A_{L}U_{F}~G_{L}~A_{F}~U_{L18}~U_{F18}~U_{M18}~A_{F}~G_{M}
P-U_{M}~U_{F18}~U_{L18}U_{F18}~A_{M}U_{L18}~U_{M18}U_{F18}~C_{M18}A_{F}~C_{M}C_{F18}~A_{L}U_{F}~G_{L}~A_{F}~U_{L18}~U_{F18}~U_{M18}~A_{F}~G_{M}
P-U_{M}~U_{F}~U_{L}U_{F3}~A_{M}U_{L3}~U_{M}U_{F}~C_{M4}A_{F}~C_{M}C_{F4}~A_{L}U~G_{L}~A_{F}~U_{L3}~U_{F}~U_{M}~A_{F}~G_{M}
P-U_{M}~U_{F}~U_{L3}U_{F3}~A_{M}U_{L3}~U_{M3}U_{F}~C_{M4}A_{F}~C_{M}C_{F}~A_{L}U~G_{L}~A_{F}~U_{L3}~U_{F3}~U_{M}~A_{F}~G_{M}
P-U_{M}~U_{F3}~U_{L3}U_{F3}~A_{M}U_{L3}~U_{M3}U_{F3}~C_{M}A_{F}~C_{M}C_{F}~A_{L}U~G_{L}~A_{F}~U_{L3}~U_{F3}~U_{M3}~A_{F}~G_{M}
P-U_{M}~U_{F3}~U_{L3}U_{F3}~A_{M}U_{L3}~U_{M3}U_{F3}~C_{M3}A_{F}~C_{M}C_{F3}~A_{L}U~G_{L}~A_{F}~U_{L3}~U_{F3}~U_{M3}~A_{F}~G_{M}
P-U_{M}~U_{F}~U_{L}U_{F12}~A_{M}U_{L12}~U_{M}U_{F}~C_{M4}A_{F}~C_{M}C_{F4}~A_{L}U~G_{L}~A_{F}~U_{L12}~U_{F}~U_{M}~A_{F}~G_{M}
P-U_{M}~U_{F}~U_{L12}U_{F12}~A_{M}U_{L12}~U_{M12}U_{F}~C_{M4}A_{F}~C_{M}C_{F}~A_{L}U~G_{L}~A_{F}~U_{L12}~U_{F12}~U_{M}~A_{F}~G_{M}
P-U_{M}~U_{F12}~U_{L12}U_{F12}~A_{M}U_{L12}~U_{M12}U_{F12}~C_{M}A_{F}~C_{M}C_{F}~A_{L}U~G_{L}~A_{F}~U_{L12}~U_{F12}~U_{M12}~A_{F}~G_{M}
P-U_{M}~U_{F12}~U_{L12}U_{F12}~A_{M}U_{L12}~U_{M12}U_{F12}~C_{M12}A_{F}~C_{M}C_{F12}~A_{L}U~G_{L}~A_{F}~U_{L12}~U_{F12}~U_{M12}~A_{F}~G_{M}
P-U_{M}~U_{F}~U_{L}U_{F18}~A_{M}U_{L18}~U_{M}U_{F}~C_{M4}A_{F}~C_{M}C_{F4}~A_{L}U~G_{L}~A_{F}~U_{L18}~U_{F}~U_{M}~A_{F}~G_{M}
P-U_{M}~U_{F}~U_{L18}U_{F18}~A_{M}U_{L18}~U_{M18}U_{F}~C_{M4}A_{F}~C_{M}C_{F}~A_{L}U~G_{L}~A_{F}~U_{L18}~U_{F18}~U_{M}~A_{F}~G_{M}
P-U_{M}~U_{F18}~U_{L18}U_{F18}~A_{M}U_{L18}~U_{M18}U_{F18}~C_{M}A_{F}~C_{M}C_{F}~A_{L}U~G_{L}~A_{F}~U_{L18}~U_{F18}~U_{M18}~A_{F}~G_{M}
P-U_{M}~U_{F18}~U_{L18}U_{F18}~A_{M}U_{L18}~U_{M18}U_{F18}~C_{M18}A_{F}~C_{M}C_{F18}~A_{L}U~G_{L}~A_{F}~U_{L18}~U_{F18}~U_{M18}~A_{F}~G_{M}
P-U_{M}~U_{F}~U_{M}U_{F}~A_{L}U_{F}~U_{M}U_{L}~C_{M}A_{F}~C_{M}C_{L}~A_{M}U_{F}~G_{M}~A_{F}~U_{L}~U_{F}~U_{L}~A_{F}~G_{M}
P-U_{M}~U_{F}~U_{M}U_{F3}~A_{L}U_{F3}~U_{M}U_{L}~C_{M4}A_{F}~C_{M}C_{L4}~A_{M}U_{F}~G_{M}~A_{F}~U_{L3}~U_{F}~U_{L}~A_{F}~G_{M}
P-U_{M}~U_{F}~U_{M3}U_{F3}~A_{L}U_{F3}~U_{M3}U_{L}~C_{M4}A_{F}~C_{M}C_{L}~A_{M}U_{F}~G_{M}~A_{F}~U_{L3}~U_{F3}~U_{L}~A_{F}~G_{M}
P-U_{M}~U_{F3}~U_{M3}U_{F3}~A_{L}U_{F3}~U_{M3}U_{L3}~C_{M}A_{F}~C_{M}C_{L}~A_{M}U_{F}~G_{M}~A_{F}~U_{L3}~U_{F3}~U_{L3}~A_{F}~G_{M}
P-U_{M}~U_{F3}~U_{M3}U_{F3}~A_{L}U_{F3}~U_{M3}U_{L3}~C_{M3}A_{F}~C_{M}C_{L3}~A_{M}U_{F}~G_{M}~A_{F}~U_{L3}~U_{F3}~U_{L3}~A_{F}~G_{M}
P-U_{M}~U_{F}~U_{M}U_{F12}~A_{L}U_{F12}~U_{M}U_{L}~C_{M4}A_{F}~C_{M}C_{L4}~A_{M}U_{F}~G_{M}~A_{F}~U_{L12}~U_{F}~U_{L}~A_{F}~G_{M}
P-U_{M}~U_{F}~U_{M12}U_{F12}~A_{L}U_{F12}~U_{M12}U_{L}~C_{M4}A_{F}~C_{M}C_{L}~A_{M}U_{F}~G_{M}~A_{F}~U_{L12}~U_{F12}~U_{L}~A_{F}~G_{M}
P-U_{M}~U_{F12}~U_{M12}U_{F12}~A_{L}U_{F12}~U_{M12}U_{L12}~C_{M}A_{F}~C_{M}C_{L}~A_{M}U_{F}~G_{M}~A_{F}~U_{L12}~U_{F12}~U_{L12}~A_{F}~G_{M}
P-U_{M}~U_{F12}~U_{M12}U_{F12}~A_{L}U_{F12}~U_{M12}U_{L12}~C_{M12}A_{F}~C_{M}C_{L12}~A_{M}U_{F}~G_{M}~A_{F}~U_{L12}~U_{F12}~U_{L12}~A_{F}~G_{M}
P-U_{M}~U_{F}~U_{M}U_{F18}~A_{L}U_{F18}~U_{M}U_{L}~C_{M4}A_{F}~C_{M}C_{L4}~A_{M}U_{F}~G_{M}~A_{F}~U_{L18}~U_{F}~U_{L}~A_{F}~G_{M}
P-U_{M}~U_{F}~U_{M18}U_{F18}~A_{L}U_{F18}~U_{M18}U_{L}~C_{M4}A_{F}~C_{M}C_{L}~A_{M}U_{F}~G_{M}~A_{F}~U_{L18}~U_{F18}~U_{L}~A_{F}~G_{M}
P-U_{M}~U_{F18}~U_{M18}U_{F18}~A_{L}U_{F18}~U_{M18}U_{L18}~C_{M}A_{F}~C_{M}C_{L}~A_{M}U_{F}~G_{M}~A_{F}~U_{L18}~U_{F18}~U_{L18}~A_{F}~G_{M}
P-U_{M}~U_{F18}~U_{M18}U_{F18}~A_{L}U_{F18}~U_{M18}U_{L18}~C_{M18}A_{F}~C_{M}C_{L18}~A_{M}U_{F}~G_{M}~A_{F}~U_{L18}~U_{F18}~U_{L18}~A_{F}~G_{M}
P-U_{M}~U_{F}~U_{M}U_{F3}~A_{L}U_{F3}~U_{M}U_{L}~C_{M4}A_{F}~C_{M}C_{L4}~A_{M}U~G_{M}~A_{F}~U_{L3}~U_{F}~U_{L}~A_{F}~G_{M}
P-U_{M}~U_{F}~U_{M3}U_{F3}~A_{L}U_{F3}~U_{M3}U_{L}~C_{M4}A_{F}~C_{M}C_{L}~A_{M}U~G_{M}~A_{F}~U_{L3}~U_{F3}~U_{L}~A_{F}~G_{M}
P-U_{M}~U_{F3}~U_{M3}U_{F3}~A_{L}U_{F3}~U_{M3}U_{L3}~C_{M}A_{F}~C_{M}C_{L}~A_{M}U~G_{M}~A_{F}~U_{L3}~U_{F3}~U_{L3}~A_{F}~G_{M}
P-U_{M}~U_{F3}~U_{M3}U_{F3}~A_{L}U_{F3}~U_{M3}U_{L3}~C_{M3}A_{F}~C_{M}C_{L3}~A_{M}U~G_{M}~A_{F}~U_{L3}~U_{F3}~U_{L3}~A_{F}~G_{M}
P-U_{M}~U_{F}~U_{M}U_{F12}~A_{L}U_{F12}~U_{M}U_{L}~C_{M4}A_{F}~C_{M}C_{L4}~A_{M}U~G_{M}~A_{F}~U_{L12}~U_{F}~U_{L}~A_{F}~G_{M}
P-U_{M}~U_{F}~U_{M12}U_{F12}~A_{L}U_{F12}~U_{M12}U_{L}~C_{M4}A_{F}~C_{M}C_{L}~A_{M}U~G_{M}~A_{F}~U_{L12}~U_{F12}~U_{L}~A_{F}~G_{M}
P-U_{M}~U_{F12}~U_{M12}U_{F12}~A_{L}U_{F12}~U_{M12}U_{L12}~C_{M}A_{F}~C_{M}C_{L}~A_{M}U~G_{M}~A_{F}~U_{L12}~U_{F12}~U_{L12}~A_{F}~G_{M}
P-U_{M}~U_{F12}~U_{M12}U_{F12}~A_{L}U_{F12}~U_{M12}U_{L12}~C_{M12}A_{F}~C_{M}C_{L12}~A_{M}U~G_{M}~A_{F}~U_{L12}~U_{F12}~U_{L12}~A_{F}~G_{M}
P-U_{M}~U_{F}~U_{M}U_{F18}~A_{L}U_{F18}~U_{M}U_{L}~C_{M4}A_{F}~C_{M}C_{L4}~A_{M}U~G_{M}~A_{F}~U_{L18}~U_{F}~U_{L}~A_{F}~G_{M}
P-U_{M}~U_{F}~U_{M18}U_{F18}~A_{L}U_{F18}~U_{M18}U_{L}~C_{M4}A_{F}~C_{M}C_{L}~A_{M}U~G_{M}~A_{F}~U_{L18}~U_{F18}~U_{L}~A_{F}~G_{M}
P-U_{M}~U_{F18}~U_{M18}U_{F18}~A_{L}U_{F18}~U_{M18}U_{L18}~C_{M}A_{F}~C_{M}C_{L}~A_{M}U~G_{M}~A_{F}~U_{L18}~U_{F18}~U_{L18}~A_{F}~G_{M}
P-U_{M}~U_{F18}~U_{M18}U_{F18}~A_{L}U_{F18}~U_{M18}U_{L18}~C_{M18}A_{F}~C_{M}C_{L18}~A_{M}U~G_{M}~A_{F}~U_{L18}~U_{F18}~U_{L18}~A_{F}~G_{M}

### Table 15

### Examples of Murine Apo-B ags-siRNA Compounds

### A. Murine Apo-B Ags-RNAi Compositions Described in the Art:

UUGAUCUAAAUGCAUUGUG

Haringsma et al. (Nucleic Acids Res 40: 4125-36, 2012) used this sequence to generate ssRNAi compounds against murine APO-B target site 6981 including the following:

P-U_{F}U_{F}G_{F}A_{F}U_{F}C_{F}U_{F}A_{F}A_{F}A_{F}U_{F}G_{F}C_{F}A_{F}U_{F}U_{F}G_{F}U_{F}G_{F}U_{M}U_{M}

Any of the 5′-end nucleosides (P-U_{F} or P-U_{M}) shown in the examples of agsRNAi compounds in Tables 15A and 15B can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-Q_{E}, 5′-VP-T_{Q} and 5′-VP-T_{B}.

Any of the nucleosides in Tables 15A and 15B with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the compounds in Tables 15A and 15B can any of those units and linkages provided for herein for overhang precursors.

B. Examples of Application of AGSD to this Compound:

TABLE 15A
Compositions of Matter Suitable for Use in vitro or
in a Subject with a Protective Carrier (5′-3′)

P-U_{F}U_{F3}G_{F}A_{F}U_{F3}C_{F4}U_{F}A_{F}A_{F}A_{F}U_{F}G_{F}C_{F4}A_{F}U_{F3}U_{F}G_{F}U_{F3}G_{F}A_{F}G_{M}
P-U_{F}U_{F}G_{F}A_{F}U_{F3}C_{F}U_{F3}A_{F}A_{F}A_{F}U_{F}G_{F}C_{F}A_{F}U_{F3}U_{F}G_{F}U_{F3}G_{F}A_{F}G_{M}
P-U_{F}U_{F}G_{F}A_{F}U_{F3}C_{F}U_{F}A_{F}A_{F}A_{F}U_{F3}G_{F}C_{F}A_{F}U_{F3}U_{F}G_{F}U_{F3}G_{F}A_{F}G_{M}
P-U_{F}U_{F}G_{F}A_{F}U_{F3}C_{F}U_{F}A_{F}A_{F}A_{F}U_{F}G_{F}C_{F}A_{F}U_{F3}U_{F3}G_{F}U_{F3}G_{F}A_{F}G_{M}
P-U_{F}U_{F3}G_{F}A_{F}U_{F}C_{F4}U_{F}A_{F}A_{F}A_{F}U_{F}G_{F4}C_{F4}A_{F}U_{F}U_{F}G_{F}U_{F3}G_{F}A_{F}G_{M}
P-U_{F}U_{F12}G_{F}A_{F}U_{F12}C_{F4}U_{F}A_{F}A_{F}A_{F}U_{F}G_{F}C_{F4}A_{F}U_{F12}U_{F}G_{F}U_{F12}G_{F}A_{F}G_{M}
P-U_{F}U_{F}G_{F}A_{F}U_{F12}C_{F}U_{F12}A_{F}A_{F}A_{F}U_{F}G_{F}C_{F}A_{F}U_{F12}U_{F}G_{F}U_{F12}G_{F}A_{F}G_{M}
P-U_{F}U_{F}G_{F}A_{F}U_{F12}C_{F}U_{F}A_{F}A_{F}A_{F}U_{F12}G_{F}C_{F}A_{F}U_{F12}U_{F}G_{F}U_{F12}G_{F}A_{F}G_{M}
P-U_{F}U_{F}G_{F}A_{F}U_{F12}C_{F}U_{F}A_{F}A_{F}A_{F}U_{F}G_{F}C_{F}A_{F}U_{F12}U_{F12}G_{F}U_{F12}G_{F}A_{F}G_{M}
P-U_{F}U_{F12}G_{F}A_{F}U_{F}C_{F4}U_{F}A_{F}A_{F}A_{F}U_{F}G_{F}C_{F4}A_{F}U_{F}U_{F}G_{F}U_{F12}G_{F}A_{F}G_{M}
P-U_{F}U_{F18}G_{F}A_{F}U_{F18}C_{F4}U_{F}A_{F}A_{F}A_{F}U_{F}G_{F}C_{F4}A_{F}U_{F18}U_{F}G_{F}U_{F18}G_{F}A_{F}G_{M}
P-U_{F}U_{F}G_{F}A_{F}U_{F18}C_{F}U_{F18}A_{F}A_{F}A_{F}U_{F}G_{F}C_{F}A_{F}U_{F18}U_{F}G_{F}U_{F18}G_{F}A_{F}G_{M}
P-U_{F}U_{F}G_{F}A_{F}U_{F18}C_{F}U_{F}A_{F}A_{F}A_{F}U_{F18}G_{F}C_{F}A_{F}U_{F18}U_{F}G_{F}U_{F18}G_{F}A_{F}G_{M}
P-U_{F}U_{F}G_{F}A_{F}U_{F18}C_{F}U_{F}A_{F}A_{F}A_{F}U_{F}G_{F}C_{F}A_{F}U_{F18}U_{F18}G_{F}U_{F18}G_{F}A_{F}G_{M}
P-U_{F}U_{F18}G_{F}A_{F}U_{F}C_{F4}U_{F}A_{F}A_{F}A_{F}U_{F}G_{F}C_{F4}A_{F}U_{F}U_{F}G_{F}U_{F18}G_{F}A_{F}G_{M}
P-U_{F}U_{F3}G_{F}A_{F}U_{F3}C_{F4}U_{F}A_{F}A_{F}A_{F}U_{F}G_{F}C_{F4}AU_{F3}U_{F}G_{F}U_{F3}G_{F}A_{F}G_{M}
P-U_{F}U_{F}G_{F}A_{F}U_{F3}C_{F}U_{F3}A_{F}A_{F}A_{F}U_{F}G_{F}C_{F}AU_{F3}U_{F}G_{F}U_{F3}G_{F}A_{F}G_{M}
P-U_{F}U_{F}G_{F}A_{F}U_{F3}C_{F}U_{F}A_{F}A_{F}A_{F}U_{F3}G_{F}C_{F}AU_{F3}U_{F}G_{F}U_{F3}G_{F}A_{F}G_{M}
P-U_{F}U_{F}G_{F}A_{F}U_{F3}C_{F}U_{F}A_{F}A_{F}A_{F}U_{F}G_{F}C_{F}AU_{F3}U_{F3}G_{F}U_{F3}G_{F}A_{F}G_{M}
P-U_{F}U_{F3}G_{F}A_{F}U_{F}C_{F4}U_{F}A_{F}A_{F}A_{F}U_{F}G_{F}C_{F4}AU_{F}U_{F}G_{F}U_{F3}G_{F}A_{F}G_{M}
P-U_{F}U_{F12}G_{F}A_{F}U_{F12}C_{F4}U_{F}A_{F}A_{F}A_{F}U_{F}G_{F}C_{F}AU_{F12}U_{F}G_{F}U_{F12}G_{F}A_{F}G_{M}
P-U_{F}U_{F}G_{F}A_{F}U_{F12}C_{F}U_{F12}A_{F}A_{F}A_{F}U_{F}G_{F}C_{F}AU_{F12}U_{F}G_{F}U_{F12}G_{F}A_{F}G_{M}
P-U_{F}U_{F}G_{F}A_{F}U_{F12}C_{F}U_{F}A_{F}A_{F}A_{F}U_{F12}G_{F}C_{F}AU_{F}U_{F12}G_{F}U_{F12}G_{F}A_{F}G_{M}
P-U_{F}U_{F}G_{F}A_{F}U_{F12}C_{F}U_{F}A_{F}A_{F}A_{F}U_{F}G_{F}C_{F}AU_{F12}U_{F12}G_{F}U_{F12}G_{F}A_{F}G_{M}
P-U_{F}U_{F12}G_{F}A_{F}U_{F}C_{F4}U_{F}A_{F}A_{F}A_{F}U_{F}G_{F}C_{F4}AU_{F}U_{F}G_{F}U_{F12}G_{F}A_{F}G_{M}
P-U_{F}U_{F18}G_{F}A_{F}U_{F18}C_{F4}U_{F}A_{F}A_{F}A_{F}U_{F}G_{F}C_{F4}AU_{F18}U_{F}G_{F}U_{F18}G_{F}A_{F}G_{M}
P-U_{F}U_{F}G_{F}A_{F}U_{F18}C_{F}U_{F18}A_{F}A_{F}A_{F}U_{F}G_{F}C_{F}AU_{F}U_{F18}G_{F}U_{F18}G_{F}A_{F}G_{M}
P-U_{F}U_{F}G_{F}A_{F}U_{F18}C_{F}U_{F}A_{F}A_{F}A_{F}U_{F18}G_{F}C_{F}AU_{F18}U_{F}G_{F}U_{F18}G_{F}A_{F}G_{M}
P-U_{F}U_{F}G_{F}A_{F}U_{F18}C_{F}U_{F}A_{F}A_{F}A_{F}U_{F}G_{F}C_{F}AU_{F18}U_{F18}G_{F}U_{F18}G_{F}A_{F}G_{M}
P-U_{F}U_{F18}G_{F}A_{F}U_{F}C_{F4}U_{F}A_{F}A_{F}A_{F}U_{F}G_{F}C_{F4}AU_{F}U_{F}G_{F}U_{F18}G_{F}A_{F}G_{M}
P-U_{F}U_{F}G_{L}A_{F}U_{F}C_{L}U_{F}A_{F}A_{L}A_{F}U_{F}G_{L}C_{F}A_{F}U_{L}U_{F}G_{L}U_{F}G_{F}A_{F}G_{M}
P-U_{F}U_{F3}G_{L}A_{F}U_{F3}C_{L4}U_{F}A_{F}A_{L}A_{F}U_{F}G_{L}C_{F4}A_{F}U_{L3}U_{F}G_{L}U_{F3}G_{F}A_{F}G_{M}
P-U_{F}U_{F}G_{L}A_{F}U_{F3}C_{L}U_{F3}A_{F}A_{L}A_{F}U_{F}G_{L}C_{F}A_{F}U_{L3}U_{F}G_{L}U_{F3}G_{F}A_{F}G_{M}
P-U_{F}U_{F}G_{L}A_{F}U_{F3}C_{L}U_{F}A_{F}A_{L}A_{F}U_{F3}G_{L}C_{F}A_{F}U_{L3}U_{F}G_{L}U_{F3}G_{F}A_{F}G_{M}
P-U_{F}U_{F}G_{L}A_{F}U_{F3}C_{L}U_{F}A_{F}A_{L}A_{F}U_{F}G_{L}C_{F}A_{F}U_{L3}U_{F3}G_{L}U_{F3}G_{F}A_{F}G_{M}
P-U_{F}U_{F3}G_{L}A_{F}U_{F}C_{L4}U_{F}A_{F}A_{L}A_{F}U_{F}G_{L}C_{F4}A_{F}U_{L}U_{F}G_{L}U_{F3}G_{F}A_{F}G_{M}
P-U_{F}U_{F12}G_{L}A_{F}U_{F12}C_{L4}U_{F}A_{F}A_{L}A_{F}U_{F}G_{L}C_{F4}A_{F}U_{L12}U_{F}G_{L}U_{F12}G_{F}A_{F}G_{M}
P-U_{F}U_{F}G_{L}A_{F}U_{F12}C_{L}U_{F12}A_{F}A_{L}A_{F}U_{F}G_{L}C_{F}A_{F}U_{L12}U_{F}G_{L}U_{F12}G_{F}A_{F}G_{M}
P-U_{F}U_{F}G_{L}A_{F}U_{F12}C_{L}U_{F}A_{F}A_{L}A_{F}U_{F12}G_{L}C_{F}A_{F}U_{L12}U_{F}G_{L}U_{F12}G_{F}A_{F}G_{M}
P-U_{F}U_{F}G_{L}A_{F}U_{F12}C_{L}U_{F}A_{F}A_{L}A_{F}U_{F}G_{L}C_{F}A_{F}U_{L12}U_{F12}G_{L}U_{F12}G_{F}A_{F}G_{M}
P-U_{F}U_{F12}G_{L}A_{F}U_{F}C_{L4}U_{F}A_{F}A_{L}A_{F}U_{F}G_{L}C_{F4}A_{F}U_{L}U_{F}G_{L}U_{F12}G_{F}A_{F}G_{M}
P-U_{F}U_{F18}G_{L}A_{F}U_{F18}C_{L4}U_{F}A_{F}A_{L}A_{F}U_{F}G_{L}C_{F4}A_{F}U_{L18}U_{F}G_{L}U_{F18}G_{F}A_{F}G_{M}
P-U_{F}U_{F}G_{L}A_{F}U_{F18}C_{L}U_{F18}A_{F}A_{L}A_{F}U_{F}G_{L}C_{F}A_{F}U_{L18}U_{F}G_{L}U_{F18}G_{F}A_{F}G_{M}
P-U_{F}U_{F}G_{L}A_{F}U_{F18}C_{L}U_{F}A_{F}A_{L}A_{F}U_{F18}G_{L}C_{F}A_{F}U_{L18}U_{F}G_{L}U_{F18}G_{F}A_{F}G_{M}
P-U_{F}U_{F}G_{L}A_{F}U_{F18}C_{L}U_{F}A_{F}A_{L}A_{F}U_{F}G_{L}C_{F}A_{F}U_{L18}U_{F18}G_{L}U_{F18}G_{F}A_{F}G_{M}
P-U_{F}U_{F18}G_{L}A_{F}U_{F}C_{L4}U_{F}A_{F}A_{L}A_{F}U_{F}G_{L}C_{F4}A_{F}U_{L}U_{F}G_{L}U_{F18}G_{F}A_{F}G_{M}
P-U_{F}U_{F3}G_{L}A_{F}U_{F3}C_{L4}U_{F}A_{F}A_{L}A_{F}U_{F}G_{L}C_{F4}AU_{L3}U_{F}G_{L}U_{F3}G_{F}A_{F}G_{M}
P-U_{F}U_{F}G_{L}A_{F}U_{F3}C_{L}U_{F3}A_{F}A_{L}A_{F}U_{F}G_{L}C_{F}AU_{L3}U_{F}G_{L}U_{F3}G_{F}A_{F}G_{M}
P-U_{F}U_{F}G_{L}A_{F}U_{F3}C_{L}U_{F}A_{F}A_{L}A_{F}U_{F3}G_{L}C_{F}AU_{L3}U_{F}G_{L}U_{F3}G_{F}A_{F}G_{M}
P-U_{F}U_{F}G_{L}A_{F}U_{F3}C_{L}U_{F}A_{F}A_{L}A_{F}U_{F}G_{L}C_{F}AU_{L3}U_{F3}G_{L}U_{F3}G_{F}A_{F}G_{M}
P-U_{F}U_{F3}G_{L}A_{F}U_{F}C_{L4}U_{F}A_{F}A_{L}A_{F}U_{F}G_{L}C_{F4}AU_{L}U_{F}G_{L}U_{F3}G_{F}A_{F}G_{M}
P-U_{F}U_{F12}G_{L}A_{F}U_{F12}C_{L4}U_{F}A_{F}A_{L}A_{F}U_{F}G_{L}C_{F4}AU_{L12}U_{F}G_{L}U_{F12}G_{F}A_{F}G_{M}
P-U_{F}U_{F}G_{L}A_{F}U_{F12}C_{L}U_{F12}A_{F}A_{L}A_{F}U_{F}G_{L}C_{F}AU_{L12}U_{F}G_{L}U_{F12}G_{F}A_{F}G_{M}
P-U_{F}U_{F}G_{L}A_{F}U_{F12}C_{L}U_{F}A_{F}A_{L}A_{F}U_{F12}G_{L}C_{F}AU_{L12}U_{F}G_{L}U_{F12}G_{F}A_{F}G_{M}
P-U_{F}U_{F}G_{L}A_{F}U_{F12}C_{L}U_{F}A_{F}A_{L}A_{F}U_{F}G_{L}C_{F}AU_{L12}U_{F12}G_{L}U_{F12}G_{F}A_{F}G_{M}
P-U_{F}U_{F12}G_{L}A_{F}U_{F}C_{L4}U_{F}A_{F}A_{L}A_{F}U_{F}G_{L}C_{F4}AU_{L}U_{F}G_{L}U_{F12}G_{F}A_{F}G_{M}
P-U_{F}U_{F18}G_{L}A_{F}U_{F18}C_{L4}U_{F}A_{F}A_{L}A_{F}U_{F}G_{L}C_{F4}AU_{L18}U_{F}G_{L}U_{F18}G_{F}A_{F}G_{M}
P-U_{F}U_{F}G_{L}A_{F}U_{F18}C_{L}U_{F18}A_{F}A_{L}A_{F}U_{F}G_{L}C_{F}AU_{L18}U_{F}G_{L}U_{F18}G_{F}A_{F}G_{M}
P-U_{F}U_{F}G_{L}A_{F}U_{F18}C_{L}U_{F}A_{F}A_{L}A_{F}U_{F18}G_{L}C_{F}AU_{L18}U_{F}G_{L}U_{F18}G_{F}A_{F}G_{M}
P-U_{F}U_{F}G_{L}A_{F}U_{F18}C_{L}U_{F}A_{F}A_{L}A_{F}U_{F}G_{L}C_{F}AU_{L18}U_{F18}G_{L}U_{F18}G_{F}A_{F}G_{M}
P-U_{F}U_{F18}G_{L}A_{F}U_{F}C_{L4}U_{F}A_{F}A_{L}A_{F}U_{F}G_{L}C_{F4}AU_{L}U_{F}G_{L}U_{F18}G_{F}A_{F}G_{M}
P-U_{F}U_{F}G_{F}A_{L}U_{F}C_{L}U_{F}A_{F}A_{L}A_{F}U_{F}G_{F}C_{L}A_{F}U_{F}U_{L}G_{F}U_{L}G_{F}A_{F}G_{M}
P-U_{F}U_{F3}G_{F}A_{L}U_{F3}C_{L4}U_{F}A_{L}A_{F}A_{F}U_{F}G_{F}C_{L4}A_{F}U_{F3}U_{L}G_{F}U_{L3}G_{F}A_{F}G_{M}
P-U_{F}U_{F}G_{F}A_{L}U_{F3}C_{L}U_{F3}A_{F}A_{L}A_{F}U_{F}G_{F}C_{L}A_{F}U_{F3}U_{L}G_{F}U_{L3}G_{F}A_{F}G_{M}
P-U_{F}U_{F}G_{F}A_{L}U_{F3}C_{L}U_{F}A_{F}A_{L}A_{F}U_{F3}G_{F}C_{L}A_{F}U_{F3}U_{L}G_{F}U_{L3}G_{F}A_{F}G_{M}
P-U_{F}U_{F}G_{F}A_{L}U_{F3}C_{L}U_{F}A_{F}A_{L}A_{F}U_{F}G_{F}C_{L}A_{F}U_{F3}U_{L3}G_{F}U_{L3}G_{F}A_{F}G_{M}
P-U_{F}U_{F3}G_{F}A_{L}U_{F}C_{L4}U_{F}A_{F}A_{L}A_{F}U_{F}G_{F}C_{L4}A_{F}U_{F}U_{L}G_{F}U_{L3}G_{F}A_{F}G_{M}
P-U_{F}U_{F12}G_{F}A_{L}U_{F12}C_{L4}U_{F}A_{F}A_{L}A_{F}U_{F}G_{F}C_{L4}A_{F}U_{F12}U_{L}G_{F}U_{L12}G_{F}A_{F}G_{M}
P-U_{F}U_{F}G_{F}A_{L}U_{F12}C_{L}U_{F12}A_{F}A_{L}A_{F}U_{F}G_{F}C_{L}A_{F}U_{F12}U_{L}G_{F}U_{L12}G_{F}A_{F}G_{M}
P-U_{F}U_{F}G_{F}A_{L}U_{F12}C_{L}U_{F}A_{F}A_{L}A_{F}U_{F12}G_{F}C_{L}A_{F}U_{F12}U_{L}G_{F}U_{L12}G_{F}A_{F}G_{M}
P-U_{F}U_{F}G_{F}A_{L}U_{F12}C_{L}U_{F}A_{F}A_{L}A_{F}U_{F}G_{F}C_{L}A_{F}U_{F12}U_{L12}G_{F}U_{L12}G_{F}A_{F}G_{M}
P-U_{F}U_{F12}G_{F}A_{L}U_{F}C_{L4}U_{F}A_{F}A_{L}A_{F}U_{F}G_{F}C_{L4}A_{F}U_{F}U_{L}G_{F}U_{L12}G_{F}A_{F}G_{M}
P-U_{F}U_{F18}G_{F}A_{L}U_{F18}C_{L4}U_{F}A_{F}A_{L}A_{F}U_{F}G_{F}C_{L4}A_{F}U_{F18}U_{L}G_{F}U_{L18}G_{F}A_{F}G_{M}
P-U_{F}U_{F}G_{F}A_{L}U_{F18}C_{L}U_{F18}A_{F}A_{L}A_{F}U_{F}G_{F}C_{L}A_{F}U_{F18}U_{L}G_{F}U_{L18}G_{F}A_{F}G_{M}
P-U_{F}U_{F}G_{F}A_{L}U_{F18}C_{L}U_{F}A_{F}A_{L}A_{F}U_{F18}G_{F}C_{L}A_{F}U_{F18}U_{L}G_{F}U_{L18}G_{F}A_{F}G_{M}
P-U_{F}U_{F}G_{F}A_{L}U_{F18}C_{L}U_{F}A_{F}A_{L}A_{F}U_{F}G_{F}C_{L}A_{F}U_{F18}U_{L18}G_{F}U_{L18}G_{F}A_{F}G_{M}
P-U_{F}U_{F18}G_{F}A_{L}U_{F}C_{L4}U_{F}A_{F}A_{L}A_{F}U_{F}G_{F}C_{L4}A_{F}U_{F}U_{L}G_{F}U_{L18}G_{F}A_{F}G_{M}
P-U_{F}U_{F3}G_{F}A_{L}U_{F3}C_{L4}U_{F}A_{F}A_{L}A_{F}U_{F}G_{F}C_{L4}AU_{F3}U_{L}G_{F}U_{L3}G_{F}A_{F}G_{M}
P-U_{F}U_{F}G_{F}A_{L}U_{F3}C_{L}U_{F3}A_{F}A_{L}A_{F}U_{F}G_{F}C_{L}AU_{F3}U_{L}G_{F}U_{L3}G_{F}A_{F}G_{M}
P-U_{F}U_{F}G_{F}A_{L}U_{F3}C_{L}U_{F}A_{F}A_{L}A_{F}U_{F3}G_{F}C_{L}AU_{F3}U_{L}G_{F}U_{L3}G_{F}A_{F}G_{M}
P-U_{F}U_{F}G_{F}A_{L}U_{F3}C_{L}U_{F}A_{F}A_{L}A_{F}U_{F}G_{F}C_{L}AU_{F3}U_{L3}G_{F}U_{L3}G_{F}A_{F}G_{M}
P-U_{F}U_{F3}G_{F}A_{L}U_{F}C_{L4}U_{F}A_{F}A_{L}A_{F}U_{F}G_{F}C_{L4}AU_{F}U_{L}G_{F}U_{L3}G_{F}A_{F}G_{M}
P-U_{F}U_{F12}G_{F}A_{L}U_{F12}C_{L4}U_{F}A_{F}A_{L}A_{F}U_{F}G_{F}C_{L4}AU_{F12}U_{L}G_{F}U_{L12}G_{F}A_{F}G_{M}
P-U_{F}U_{F}G_{F}A_{L}U_{F12}C_{L}U_{F12}A_{F}A_{L}A_{F}U_{F}G_{F}C_{L}AU_{F12}U_{L}G_{F}U_{L12}G_{F}A_{F}G_{M}
P-U_{F}U_{F}G_{F}A_{L}U_{F12}C_{L}U_{F}A_{F}A_{L}A_{F}U_{F12}G_{F}C_{L}AU_{F12}U_{L}G_{F}U_{L12}G_{F}A_{F}G_{M}
P-U_{F}U_{F}G_{F}A_{L}U_{F12}C_{L}U_{F}A_{F}A_{L}A_{F}U_{F}G_{F}C_{L}AU_{F12}U_{L12}G_{F}U_{L12}G_{F}A_{F}G_{M}
P-U_{F}U_{F12}G_{F}A_{L}U_{F}C_{L4}U_{F}A_{F}A_{L}A_{F}U_{F}G_{F}C_{L4}AU_{F}U_{L}G_{F}U_{L12}G_{F}A_{F}G_{M}
P-U_{F}U_{F18}G_{F}A_{L}U_{F18}C_{L4}U_{F}A_{F}A_{L}A_{F}U_{F}G_{F}C_{L4}AU_{F18}U_{L}G_{F}U_{L18}G_{F}A_{F}G_{M}
P-U_{F}U_{F}G_{F}A_{L}U_{F18}C_{L}U_{F18}A_{F}A_{L}A_{F}U_{F}G_{F}C_{L}AU_{F18}U_{L}G_{F}U_{L18}G_{F}A_{F}G_{M}
P-U_{F}U_{F}G_{F}A_{L}U_{F18}C_{L}U_{F}A_{F}A_{L}A_{F}U_{F18}G_{F}C_{L}AU_{F18}U_{L}G_{F}U_{L18}G_{F}A_{F}G_{M}
P-U_{F}U_{F}G_{F}A_{L}U_{F18}C_{L}U_{F}A_{F}A_{L}A_{F}U_{F}G_{F}C_{L}AU_{F18}U_{L18}G_{F}U_{L18}G_{F}A_{F}G_{M}
P-U_{F}U_{F18}G_{F}A_{L}U_{F}C_{L4}U_{F}A_{F}A_{L}A_{F}U_{F}G_{F}C_{L4}AU_{F}U_{L}G_{F}U_{L18}G_{F}A_{F}G_{M}
P-U_{F}U_{F}G_{F}A_{L}U_{F}C_{F}U_{F}A_{L}A_{F}A_{L}U_{F}G_{L}C_{F}A_{F}U_{L}U_{F}G_{L}U_{F}G_{F}A_{F}G_{M}
P-U_{F}U_{F3}G_{F}A_{L}U_{F3}C_{F4}U_{F}A_{L}A_{F}A_{L}U_{F}G_{L}C_{F4}A_{F}U_{L3}U_{F}G_{L}U_{F3}G_{F}A_{F}G_{M}
P-U_{F}U_{F}G_{F}A_{L}U_{F3}C_{F}U_{F3}A_{L}A_{F}A_{L}U_{F}G_{L}C_{F}A_{F}U_{L3}U_{F}G_{L}U_{F3}G_{F}A_{F}G_{M}
P-U_{F}U_{F}G_{F}A_{L}U_{F3}C_{F}U_{F}A_{L}A_{F}A_{L}U_{F3}G_{L}C_{F}A_{F}U_{L3}U_{F}G_{L}U_{F3}G_{F}A_{F}G_{M}
P-U_{F}U_{F}G_{F}A_{L}U_{F3}C_{F}U_{F}A_{L}A_{F}A_{L}U_{F}G_{L}C_{F}A_{F}U_{L3}U_{F3}G_{L}U_{F3}G_{F}A_{F}G_{M}
P-U_{F}U_{F3}G_{F}A_{L}U_{F}C_{F4}U_{F}A_{L}A_{F}A_{L}U_{F}G_{L}C_{F4}A_{F}U_{L}U_{F}G_{L}U_{F3}G_{F}A_{F}G_{M}
P-U_{F}U_{F12}G_{F}A_{L}U_{F12}C_{F4}U_{F}A_{L}A_{F}A_{L}U_{F}G_{L}C_{F4}A_{F}U_{L12}U_{F}G_{L}U_{F12}G_{F}A_{F}G_{M}
P-U_{F}U_{F}G_{F}A_{L}U_{F12}C_{F}U_{F12}A_{L}A_{F}A_{L}U_{F}G_{L}C_{F}A_{F}U_{L12}U_{F}G_{L}U_{F12}G_{F}A_{F}G_{M}
P-U_{F}U_{F}G_{F}A_{L}U_{F12}C_{F}U_{F}A_{L}A_{F}A_{L}U_{F12}G_{L}C_{F}A_{F}U_{L12}U_{F}G_{L}U_{F12}G_{F}A_{F}G_{M}
P-U_{F}U_{F}G_{F}A_{L}U_{F12}C_{F}U_{F}A_{L}A_{F}A_{L}U_{F}G_{L}C_{F}A_{F}U_{L12}U_{F12}G_{L}U_{F12}G_{F}A_{F}G_{M}
P-U_{F}U_{F12}G_{F}A_{L}U_{F}C_{F4}U_{F}A_{L}A_{F}A_{L}U_{F}G_{L}C_{F4}A_{F}U_{L}U_{F}G_{L}U_{F12}G_{F}A_{F}G_{M}
P-U_{F}U_{F18}G_{F}A_{L}U_{F18}C_{F4}U_{F}A_{L}A_{F}A_{L}U_{F}G_{L}C_{F4}A_{F}U_{L18}U_{F}G_{L}U_{F18}G_{F}A_{F}G_{M}
P-U_{F}U_{F}G_{F}A_{L}U_{F18}C_{F}U_{F18}A_{L}A_{F}A_{L}U_{F}G_{L}C_{F}A_{F}U_{L18}U_{F}G_{L}U_{F18}G_{F}A_{F}G_{M}
P-U_{F}U_{F}G_{F}A_{L}U_{F18}C_{F}U_{F}A_{L}A_{F}A_{L}U_{F18}G_{L}C_{F}A_{F}U_{L18}U_{F}G_{L}U_{F18}G_{F}A_{F}G_{M}
P-U_{F}U_{F}G_{F}A_{L}U_{F18}C_{F}U_{F}A_{L}A_{F}A_{L}U_{F}G_{L}C_{F}A_{F}U_{L18}U_{F18}G_{L}U_{F18}G_{F}A_{F}G_{M}
P-U_{F}U_{F18}G_{F}A_{L}U_{F}C_{F4}U_{F}A_{L}A_{F}A_{L}U_{F}G_{L}C_{F4}A_{F}U_{L}U_{F}G_{L}U_{F18}G_{F}A_{F}G_{M}
P-U_{F}U_{F3}G_{F}A_{L}U_{F3}C_{F4}U_{F}A_{L}A_{F}A_{L}U_{F}G_{L}C_{F4}AU_{L3}U_{F}G_{L}U_{F3}G_{F}A_{F}G_{M}
P-U_{F}U_{F}G_{F}A_{L}U_{F3}C_{F}U_{F3}A_{L}A_{F}A_{L}U_{F}G_{L}C_{F}AU_{L3}U_{F}G_{L}U_{F3}G_{F}A_{F}G_{M}
P-U_{F}U_{F}G_{F}A_{L}U_{F3}C_{F}U_{F}A_{L}A_{F}A_{L}U_{F3}G_{L}C_{F}AU_{L3}U_{F}G_{L}U_{F3}G_{F}A_{F}G_{M}
P-U_{F}U_{F}G_{F}A_{L}U_{F3}C_{F}U_{F}A_{L}A_{F}A_{L}U_{F}G_{L}C_{F}AU_{L3}U_{F3}G_{L}U_{F3}G_{F}A_{F}G_{M}
P-U_{F}U_{F3}G_{F}A_{L}U_{F}C_{F4}U_{F}A_{L}A_{F}A_{L}U_{F}G_{L}C_{F4}AU_{L}U_{F}G_{L}U_{F3}G_{F}A_{F}G_{M}
P-U_{F}U_{F12}G_{F}A_{L}U_{F12}C_{F4}U_{F}A_{L}A_{F}A_{L}U_{F}G_{L}C_{F4}AU_{L12}U_{F}G_{L}U_{F12}G_{F}A_{F}G_{M}
P-U_{F}U_{F}G_{F}A_{L}U_{F12}C_{F}U_{F12}A_{L}A_{F}A_{L}U_{F}G_{L}C_{F}AU_{L12}U_{F}G_{L}U_{F12}G_{F}A_{F}G_{M}
P-U_{F}U_{F}G_{F}A_{L}U_{F12}C_{F}U_{F}A_{L}A_{F}A_{L}U_{F12}G_{L}C_{F}AU_{L12}U_{F}G_{L}U_{F12}G_{F}A_{F}G_{M}
P-U_{F}U_{F}G_{F}A_{L}U_{F12}C_{F}U_{F}A_{L}A_{F}A_{L}U_{F}G_{L}C_{F}AU_{L12}U_{F12}G_{L}U_{F12}G_{F}A_{F}G_{M}
P-U_{F}U_{F12}G_{F}A_{L}U_{F}C_{F4}U_{F}A_{L}A_{F}A_{L}U_{F}G_{L}C_{F4}AU_{L}U_{F}G_{L}U_{F12}G_{F}A_{F}G_{M}
P-U_{F}U_{F18}G_{F}A_{L}U_{F18}C_{F4}U_{F}A_{L}A_{F}A_{L}U_{F}G_{L}C_{F4}AU_{L18}U_{F}G_{L}U_{F18}G_{F}A_{F}G_{M}
P-U_{F}U_{F}G_{F}A_{L}U_{F18}C_{F}U_{F18}A_{L}A_{F}A_{L}U_{F}G_{L}C_{F}AU_{L18}U_{F}G_{L}U_{F18}G_{F}A_{F}G_{M}
P-U_{F}U_{F}G_{F}A_{L}U_{F18}C_{F}U_{F}A_{L}A_{F}A_{L}U_{F18}G_{L}C_{F}AU_{L18}U_{F}G_{L}U_{F18}G_{F}A_{F}G_{M}
P-U_{F}U_{F}G_{F}A_{L}U_{F18}C_{F}U_{F}A_{L}A_{F}A_{L}U_{F}G_{L}C_{F}AU_{L18}U_{F18}G_{L}U_{F18}G_{F}A_{F}G_{M}
P-U_{F}U_{F18}G_{F}A_{L}U_{F}C_{F4}U_{F}A_{L}A_{F}A_{L}U_{F}G_{L}C_{F4}AU_{L}U_{F}G_{L}U_{F18}G_{F}A_{F}G_{M}

TABLE 15B
Compositions of Matter Suitable for Administration to a Subject
with or without a Protective Carrier and for Any Other use
as Provided for Herein (5′-3′)

P-U_{M}~U_{F3}~G_{M}A_{F}~U_{M3}C_{F4}~U_{M}A_{F}~A_{M}A_{F}~U_{M}G_{F}~C_{M4}A_{F}~U_{M3}~U_{F}~G_{M}~U_{F3}~G_{M}~A_{F}~G_{M}
P-U_{M}~U_{F}~G_{M}A_{F}~U_{M3}C_{F}~U_{M3}A_{F}~A_{M}A_{F}~U_{M}G_{F}~C_{M}A_{F}~U_{M3}~U_{F}~G_{M}~U_{F3}~G_{M}~A_{F}~G_{M}
P-U_{M}~U_{F}~G_{M}A_{F}~U_{M3}C_{F}~U_{M}A_{F}~A_{M}A_{F}~U_{M3}G_{F}~C_{M}A_{F}~U_{M3}~U_{F}~G_{M}~U_{F3}~G_{M}~A_{F}~G_{M}
P-U_{M}~U_{F}~G_{M}A_{F}~U_{M3}C_{F}~U_{M}A_{F}~A_{M}A_{F}~U_{M}G_{F}~C_{M}A_{F}~U_{M3}~U_{F3}~G_{M}~U_{F3}~G_{M}~A_{F}~G_{M}
P-U_{M}~U_{F3}~G_{M}A_{F}~U_{M}C_{F4}~U_{M}A_{F}~A_{M}A_{F}~U_{M}G_{F}~C_{M4}A_{F}~U_{M}~U_{F}~G_{M}~U_{F3}~G_{M}~A_{F}~G_{M}
P-U_{M}~U_{F3}~G_{M}A_{F}~U_{M3}C_{F4}~U_{M3}A_{F}~A_{M}A_{F}~U_{M3}G_{F}~C_{M4}A_{F}~U_{M3}~U_{F3}~G_{M}~U_{F3}~G_{M}~A_{F}~G_{M}
P-U_{M}~U_{F12}~G_{M}A_{F}~U_{M12}C_{F4}~U_{M}A_{F}~A_{M}A_{F}~U_{M}G_{F}~C_{M4}A_{F}~U_{M12}~U_{F}~G_{M}~U_{F12}~G_{M}~A_{F}~G_{M}
P-U_{M}~U_{F}~G_{M}A_{F}~U_{M12}C_{F}~U_{M12}A_{F}~A_{M}A_{F}~U_{M}G_{F}~C_{M}A_{F}~U_{M12}~U_{F}~G_{M}~U_{F12}~G_{M}~A_{F}~G_{M}
P-U_{M}~U_{F}~G_{M}A_{F}~U_{M12}C_{F}~U_{M}A_{F}~A_{M}A_{F}~U_{M12}G_{F}~C_{M}A_{F}~U_{M12}~U_{F}~G_{M}~U_{F12}~G_{M}~A_{F}~G_{M}
P-U_{M}~U_{F}~G_{M}A_{F}~U_{M12}C_{F}~U_{M}A_{F}~A_{M}A_{F}~U_{M}G_{F}~C_{M}A_{F}~U_{M12}~U_{F12}~G_{M}~U_{F12}~G_{M}~A_{F}~G_{M}
P-U_{M}~U_{F12}~G_{M}A_{F}~U_{M}C_{F4}~U_{M}A_{F}~A_{M}A_{F}~U_{M}G_{F}~C_{M4}A_{F}~U_{M}~U_{F}~G_{M}~U_{F12}~G_{M}~A_{F}~G_{M}
P-U_{M}~U_{F12}~G_{M}A_{F}~U_{M12}C_{F4}~U_{M12}A_{F}~A_{M}A_{F}~U_{M12}G_{F}~C_{M4}A_{F}~U_{M12}~U_{F12}~G_{M}~U_{F12}~G_{M}~A_{F}~G_{M}
P-U_{M}~U_{F18}~G_{M}A_{F}~U_{M18}C_{F4}~U_{M}A_{F}~A_{M}A_{F}~U_{M}G_{F}~C_{M4}A_{F}~U_{M18}~U_{F}~G_{M}~U_{F18}~G_{M}~A_{F}~G_{M}
P-U_{M}~U_{F}~G_{M}A_{F}~U_{M18}C_{F}~U_{M18}A_{F}~A_{M}A_{F}~U_{M}G_{F}~C_{M}A_{F}~U_{M18}~U_{F}~G_{M}~U_{F18}~G_{M}~A_{F}~G_{M}
P-U_{M}~U_{F}~G_{M}A_{F}~U_{M18}C_{F}~U_{M}A_{F}~A_{M}A_{F}~U_{M18}G_{F}~C_{M}A_{F}~U_{M18}~U_{F}~G_{M}~U_{F18}~G_{M}~A_{F}~G_{M}
P-U_{M}~U_{F}~G_{M}A_{F}~U_{M18}C_{F}~U_{M}A_{F}~A_{M}A_{F}~U_{M}G_{F}~C_{M}A_{F}~U_{M18}~U_{F18}~G_{M}~U_{F18}~G_{M}~A_{F}~G_{M}
P-U_{M}~U_{F18}~G_{M}A_{F}~U_{M}C_{F4}~U_{M}A_{F}~A_{M}A_{F}~U_{M}G_{F}~C_{M4}A_{F}~U_{M}~U_{F}~G_{M}~U_{F18}~G_{M}~A_{F}~G_{M}
P-U_{M}~U_{F18}~G_{M}A_{F}~U_{M18}C_{F4}~U_{M18}A_{F}~A_{M}A_{F}~U_{M18}G_{F}~C_{M4}A_{F}~U_{M18}~U_{F18}~G_{M}~U_{F18}~G_{M}~A_{F}~G_{M}
P-U_{M}~U_{F3}~G_{M}A_{F}~U_{M3}C_{F4}~U_{M}A_{F}~A_{M}A_{F}~U_{M}G_{F}~C_{M4}A~U_{M3}~U_{F}~G_{M}~U_{F3}~G_{M}~A_{F}~G_{M}
P-U_{M}~U_{F}~G_{M}A_{F}~U_{M3}C_{F}~U_{M3}A_{F}~A_{M}A_{F}~U_{M}G_{F}~C_{M}A~U_{M3}~U_{F}~G_{M}~U_{F3}~G_{M}~A_{F}~G_{M}
P-U_{M}~U_{F}~G_{M}A_{F}~U_{M3}C_{F}~U_{M}A_{F}~A_{M}A_{F}~U_{M3}G_{F}~C_{M}A~U_{M3}~U_{F}~G_{M}~U_{F3}~G_{M}~A_{F}~G_{M}
P-U_{M}~U_{F}~G_{M}A_{F}~U_{M3}C_{F}~U_{M}A_{F}~A_{M}A_{F}~U_{M}G_{F}~C_{M}A~U_{M3}~U_{F3}~G_{M}~U_{F3}~G_{M}~A_{F}~G_{M}
P-U_{M}~U_{F3}~G_{M}A_{F}~U_{M}C_{F4}~U_{M}A_{F}~A_{M}A_{F}~U_{M}G_{F}~C_{M4}A~U_{M}~U_{F}~G_{M}~U_{F3}~G_{M}~A_{F}~G_{M}
P-U_{M}~U_{F3}~G_{M}A_{F}~U_{M3}C_{F4}~U_{M3}A_{F}~A_{M}A_{F}~U_{M3}G_{F}~C_{M4}A~U_{M3}~U_{F3}~G_{M}~U_{F3}~G_{M}~A_{F}~G_{M}
P-U_{M}~U_{F12}~G_{M}A_{F}~U_{M12}C_{F4}~U_{M}A_{F}~A_{M}A_{F}~U_{M}G_{F}~C_{M4}A~U_{M12}~U_{F}~G_{M}~U_{F12}~G_{M}~A_{F}~G_{M}
P-U_{M}~U_{F}~G_{M}A_{F}~U_{M12}C_{F}~U_{M12}A_{F}~A_{M}A_{F}~U_{M}G_{F}~C_{M}A~U_{M12}~U_{F}~G_{M}~U_{F12}~G_{M}~A_{F}~G_{M}
P-U_{M}~U_{F}~G_{M}A_{F}~U_{M12}C_{F}~U_{M}A_{F}~A_{M}A_{F}~U_{M12}G_{F}~C_{M}A~U_{M12}~U_{F}~G_{M}~U_{F12}~G_{M}~A_{F}~G_{M}
P-U_{M}~U_{F}~G_{M}A_{F}~U_{M12}C_{F}~U_{M}A_{F}~A_{M}A_{F}~U_{M}G_{F}~C_{M}A~U_{M12}~U_{F12}~G_{M}~U_{F12}~G_{M}~A_{F}~G_{M}
P-U_{M}~U_{F12}~G_{M}A_{F}~U_{M}C_{F4}~U_{M}A_{F}~A_{M}A_{F}~U_{M}G_{F}~C_{M4}A~U_{M}~U_{F}~G_{M}~U_{F12}~G_{M}~A_{F}~G_{M}
P-U_{M}~U_{F12}~G_{M}A_{F}~U_{M12}C_{F4}~U_{M12}A_{F}~A_{M}A_{F}~U_{M12}G_{F}~C_{M4}A~U_{M12}~U_{F12}~G_{M}~U_{F12}~G_{M}~A_{F}~G_{M}
P-U_{M}~U_{F18}~G_{M}A_{F}~U_{M18}C_{F4}~U_{M}A_{F}~A_{M}A_{F}~U_{M}G_{F}~C_{M4}A~U_{M18}~U_{F}~G_{M}~U_{F18}~G_{M}~A_{F}~G_{M}
P-U_{M}~U_{F}~G_{M}A_{F}~U_{M18}C_{F}~U_{M18}A_{F}~A_{M}A_{F}~U_{M}G_{F}~C_{M}A~U_{M18}~U_{F}~G_{M}~U_{F18}~G_{M}~A_{F}~G_{M}
P-U_{M}~U_{F}~G_{M}A_{F}~U_{M18}C_{F}~U_{M}A_{F}~A_{M}A_{F}~U_{M18}G_{F}~C_{M}A~U_{M18}~U_{F}~G_{M}~U_{F18}~G_{M}~A_{F}~G_{M}
P-U_{M}~U_{F}~G_{M}A_{F}~U_{M18}C_{F}~U_{M}A_{F}~A_{M}A_{F}~U_{M}G_{F}~C_{M}A~U_{M18}~U_{F18}~G_{M}~U_{F18}~G_{M}~A_{F}~G_{M}
P-U_{M}~U_{F18}~G_{M}A_{F}~U_{M}C_{F4}~U_{M}A_{F}~A_{M}A_{F}~U_{M}G_{F}~C_{M4}A~U_{M}~U_{F}~G_{M}~U_{F18}~G_{M}~A_{F}~G_{M}
P-U_{M}~U_{F18}~G_{M}A_{F}~U_{M18}C_{F4}~U_{M18}A_{F}~A_{M}A_{F}~U_{M18}G_{F}~C_{M4}A~U_{M18}~U_{F18}~G_{M}~U_{F18}~G_{M}~A_{F}~G_{M}
P-U_{M}~U_{F}~G_{L}A_{F}~U_{M}C_{L}~U_{M}A_{F}~A_{L}A_{F}~U_{M}~G_{L}~C_{M}~A_{F}~U_{L}~U_{F}~G_{L}~U_{F}~G_{M}~A_{F}~G_{M}
P-U_{M}~U_{F}~G_{L}A_{F}~U_{M}C_{L}~U_{M}A_{F}~A_{L}A_{F}~U_{M}~G_{L}~C_{M}A~U_{L}~U_{F}~G_{L}~U_{F}~G_{M}~A_{F}~G_{M}
P-U_{M}~U_{F3}~G_{L}A_{F}~U_{M3}C_{L4}~U_{M}A_{F}~A_{L}A_{F}~U_{M}G_{L}~C_{M4}A_{F}~U_{L3}~U_{F}~G_{L}~U_{F3}~G_{M}~A_{F}~G_{M}
P-U_{M}~U_{F}~G_{L}A_{F}~U_{M3}C_{L}~U_{M3}A_{F}~A_{L}A_{F}~U_{M}G_{L}~C_{M}A_{F}~U_{L3}~U_{F}~G_{L}~U_{F3}~G_{M}~A_{F}~G_{M}
P-U_{M}~U_{F}~G_{L}A_{F}~U_{M3}C_{L}~U_{M}A_{F}~A_{L}A_{F}~U_{M3}G_{L}~C_{M}A_{F}~U_{L3}~U_{F}~G_{L}~U_{F3}~G_{M}~A_{F}~G_{M}
P-U_{M}~U_{F}~G_{L}A_{F}~U_{M3}C_{L}~U_{M}A_{F}~A_{L}A_{F}~U_{M}G_{L}~C_{M}A_{F}~U_{L3}~U_{F3}~G_{L}~U_{F3}~G_{M}~A_{F}~G_{M}
P-U_{M}~U_{F3}~G_{L}A_{F}~U_{M}C_{L4}~U_{M}A_{F}~A_{L}A_{F}~U_{M}G_{L}~C_{M4}A_{F}~U_{L}~U_{F}~G_{L}~U_{F3}~G_{M}~A_{F}~G_{M}
P-U_{M}~U_{F3}~G_{L}A_{F}~U_{M3}C_{L4}~U_{M3}A_{F}~A_{L}A_{F}~U_{M3}G_{L}~C_{M4}A_{F}~U_{L3}~U_{F3}~G_{L}~U_{F3}~G_{M}~A_{F}~G_{M}
P-U_{M}~U_{F12}~G_{L}A_{F}~U_{M12}C_{L4}~U_{M}A_{F}~A_{L}A_{F}~U_{M}G_{L}~C_{M4}A_{F}~U_{L12}~U_{F}~G_{L}~U_{F12}~G_{M}~A_{F}~G_{M}
P-U_{M}~U_{F}~G_{L}A_{F}~U_{M12}C_{L}~U_{M12}A_{F}~A_{L}A_{F}~U_{M}G_{L}~C_{M}A_{F}~U_{L12}~U_{F}~G_{L}~U_{F12}~G_{M}~A_{F}~G_{M}
P-U_{M}~U_{F}~G_{L}A_{F}~U_{M12}C_{L}~U_{M}A_{F}~A_{L}A_{F}~U_{M12}G_{L}~C_{M}A_{F}~U_{L12}~U_{F}~G_{L}~U_{F12}~G_{M}~A_{F}~G_{M}
P-U_{M}~U_{F}~G_{L}A_{F}~U_{M12}C_{L}~U_{M}A_{F}~A_{L}A_{F}~U_{M}G_{L}~C_{M}A_{F}~U_{L12}~U_{F12}~G_{L}~U_{F12}~G_{M}~A_{F}~G_{M}
P-U_{M}~U_{F12}~G_{L}A_{F}~U_{M}C_{L4}~U_{M}A_{F}~A_{L}A_{F}~U_{M}G_{L}~C_{M4}A_{F}~U_{L}~U_{F}~G_{L}~U_{F12}~G_{M}~A_{F}~G_{M}
P-U_{M}~U_{F12}~G_{L}A_{F}~U_{M12}C_{L4}~U_{M12}A_{F}~A_{L}A_{F}~U_{M12}G_{L}~C_{M4}A_{F}~U_{L12}~U_{F12}~G_{L}~U_{F12}~G_{M}~A_{F}~G_{M}
P-U_{M}~U_{F18}~G_{L}A_{F}~U_{M18}C_{L4}~U_{M}A_{F}~A_{L}A_{F}~U_{M}G_{L}~C_{M4}A_{F}~U_{L18}~U_{F}~G_{L}~U_{F18}~G_{M}~A_{F}~G_{M}
P-U_{M}~U_{F}~G_{L}A_{F}~U_{M18}C_{L}~U_{M18}A_{F}~A_{L}A_{F}~U_{M}G_{L}~C_{M}A_{F}~U_{L18}~U_{F}~G_{L}~U_{F18}~G_{M}~A_{F}~G_{M}
P-U_{M}~U_{F}~G_{L}A_{F}~U_{M18}C_{L}~U_{M}A_{F}~A_{L}A_{F}~U_{M18}G_{L}~C_{M}A_{F}~U_{L18}~U_{F}~G_{L}~U_{F18}~G_{M}~A_{F}~G_{M}
P-U_{M}~U_{F}~G_{L}A_{F}~U_{M18}C_{L}~U_{M}A_{F}~A_{L}A_{F}~U_{M}G_{L}~C_{M}A_{F}~U_{L18}~U_{F18}~G_{L}~U_{F18}~G_{M}~A_{F}~G_{M}
P-U_{M}~U_{F18}~G_{L}A_{F}~U_{M}C_{L4}~U_{M}A_{F}~A_{L}A_{F}~U_{M}G_{L}~C_{M4}A_{F}~U_{L}~U_{F}~G_{L}~U_{F18}~G_{M}~A_{F}~G_{M}
P-U_{M}~U_{F18}~G_{L}A_{F}~U_{M18}C_{L4}~U_{M18}A_{F}~A_{L}A_{F}~U_{M18}G_{L}~C_{M4}A_{F}~U_{L18}~U_{F18}~G_{L}~U_{F18}~G_{M}~A_{F}~G_{M}
P-U_{M}~U_{F3}~G_{L}A_{F}~U_{M3}C_{L4}~U_{M}A_{F}~A_{L}A_{F}~U_{M}G_{L}~C_{M4}A~U_{L3}~U_{F}~G_{L}~U_{F3}~G_{M}~A_{F}~G_{M}
P-U_{M}~U_{F}~G_{L}A_{F}~U_{M3}C_{L}~U_{M3}A_{F}~A_{L}A_{F}~U_{M}G_{L}~C_{M}A~U_{L3}~U_{F}~G_{L}~U_{F3}~G_{M}~A_{F}~G_{M}
P-U_{M}~U_{F}~G_{L}A_{F}~U_{M3}C_{L}~U_{M}A_{F}~A_{L}A_{F}~U_{M3}G_{L}~C_{M}A~U_{L3}~U_{F}~G_{L}~U_{F3}~G_{M}~A_{F}~G_{M}
P-U_{M}~U_{F}~G_{L}A_{F}~U_{M3}C_{L}~U_{M}A_{F}~A_{L}A_{F}~U_{M}G_{L}~C_{M}A~U_{L3}~U_{F3}~G_{L}~U_{F3}~G_{M}~A_{F}~G_{M}
P-U_{M}~U_{F3}~G_{L}A_{F}~U_{M}C_{L4}~U_{M}A_{F}~A_{L}A_{F}~U_{M}G_{L}~C_{M4}A~U_{L}~U_{F}~G_{L}~U_{F3}~G_{M}~A_{F}~G_{M}
P-U_{M}~U_{F3}~G_{L}A_{F}~U_{M3}C_{L4}~U_{M3}A_{F}~A_{L}A_{F}~U_{M3}G_{L}~C_{M4}A~U_{L3}~U_{F3}~G_{L}~U_{F3}~G_{M}~A_{F}~G_{M}
P-U_{M}~U_{F12}~G_{L}A_{F}~U_{M12}C_{L4}~U_{M}A_{F}~A_{L}A_{F}~U_{M}G_{L}~C_{M4}A~U_{L12}~U_{F}~G_{L}~U_{F12}~G_{M}~A_{F}~G_{M}
P-U_{M}~U_{F}~G_{L}A_{F}~U_{M12}C_{L}~U_{M12}A_{F}~A_{L}A_{F}~U_{M}G_{L}~C_{M}A~U_{L12}~U_{F}~G_{L}~U_{F12}~G_{M}~A_{F}~G_{M}
P-U_{M}~U_{F}~G_{L}A_{F}~U_{M12}C_{L}~U_{M}A_{F}~A_{L}A_{F}~U_{M12}G_{L}~C_{M}A~U_{L12}~U_{F}~G_{L}~U_{F12}~G_{M}~A_{F}~G_{M}
P-U_{M}~U_{F}~G_{L}A_{F}~U_{M12}C_{L}~U_{M}A_{F}~A_{L}A_{F}~U_{M}G_{L}~C_{M}A~U_{L12}~U_{F12}~G_{L}~U_{F12}~G_{M}~A_{F}~G_{M}
P-U_{M}~U_{F12}~G_{L}A_{F}~U_{M}C_{L4}~U_{M}A_{F}~A_{L}A_{F}~U_{M}G_{L}~C_{M4}A~U_{L}~U_{F}~G_{L}~U_{F12}~G_{M}~A_{F}~G_{M}
P-U_{M}~U_{F12}~G_{L}A_{F}~U_{M12}C_{L4}~U_{M12}A_{F}~A_{L}A_{F}~U_{M12}G_{L}~C_{M4}A~U_{L12}~U_{F12}~G_{L}~U_{F12}~G_{M}~A_{F}~G_{M}
P-U_{M}~U_{F18}~G_{L}A_{F}~U_{M18}C_{L4}~U_{M}A_{F}~A_{L}A_{F}~U_{M}G_{L}~C_{M4}A~U_{L18}~U_{F}~G_{L}~U_{F18}~G_{L}~G_{M}~A_{F}~G_{M}
P-U_{M}~U_{F}~G_{L}A_{F}~U_{M18}C_{L}~U_{M18}A_{F}~A_{L}A_{F}~U_{M}G_{L}~C_{M}A~U_{L18}~U_{F}~G_{L}~U_{F18}~G_{M}~A_{F}~G_{M}
P-U_{M}~U_{F}~G_{L}A_{F}~U_{M18}C_{L}~U_{M}A_{F}~A_{L}A_{F}~U_{M18}G_{L}~C_{M}A~U_{L18}~U_{F}~G_{L}~U_{F18}~G_{M}~A_{F}~G_{M}
P-U_{M}~U_{F}~G_{L}A_{F}~U_{M18}C_{L}~U_{M}A_{F}~A_{L}A_{F}~U_{M}G_{L}~C_{M}A~U_{L18}~U_{F18}~G_{L}~U_{F18}~G_{M}~A_{F}~G_{M}
P-U_{M}~U_{F18}~G_{L}A_{F}~U_{M}C_{L4}~U_{M}A_{F}~A_{L}A_{F}~U_{M}G_{L}~C_{M4}A~U_{L}~U_{F}~G_{L}~U_{F18}~G_{M}~A_{F}~G_{M}
P-U_{M}~U_{F18}~G_{L}A_{F}~U_{M18}C_{L4}~U_{M18}A_{F}~A_{L}A_{F}~U_{M18}G_{L}~C_{M4}A~U_{L18}~U_{F18}~G_{L}~U_{F18}~G_{M}~A_{F}~G_{M}
P-U_{M}~U_{F}~G_{M}A_{L}~U_{M}C_{L}~U_{M}A_{F}~A_{L}A_{F}~U_{M}G_{F}~C_{L}A_{F}~U_{M}~U_{L}~G_{M}~U_{L}~G_{M}~A_{F}~G_{M}
P-U_{M}~U_{F}~G_{M}A_{L}~U_{M}C_{L}~U_{M}A_{F}~A_{L}A_{F}~U_{M}G_{F}~C_{L}A~U_{M}~U_{L}~G_{M}~U_{L}~G_{M}~A_{F}~G_{M}
P-U_{M}~U_{F3}~G_{M}A_{L}~U_{M3}C_{L4}~U_{M}A_{F}~A_{L}A_{F}~U_{M}G_{F}~C_{L4}A_{F}~U_{M3}~U_{L}~G_{M}~U_{L3}~G_{M}~A_{F}~G_{M}
P-U_{M}~U_{F}~G_{M}A_{L}~U_{M3}C_{L}~U_{M3}A_{F}~A_{L}A_{F}~U_{M}G_{F}~C_{L}A_{F}~U_{M3}~U_{L}~G_{M}~U_{L3}~G_{M}~A_{F}~G_{M}
P-U_{M}~U_{F}~G_{M}A_{L}~U_{M3}C_{L}~U_{M}A_{F}~A_{L}A_{F}~U_{M3}G_{F}~C_{L}A_{F}~U_{M3}~U_{L}~G_{M}~U_{L3}~G_{M}~A_{F}~G_{M}
P-U_{M}~U_{F}~G_{M}A_{L}~U_{M3}C_{L}~U_{M}A_{F}~A_{L}A_{F}~U_{M}G_{F}~C_{L}A_{F}~U_{M3}~U_{L3}~G_{M}~U_{L3}~G_{M}~A_{F}~G_{M}
P-U_{M}~U_{F3}~G_{M}A_{L}~U_{M}C_{L4}~U_{M}A_{F}~A_{L}A_{F}~U_{M}G_{F}~C_{L4}A_{F}~U_{M}~U_{L}~G_{M}~U_{L3}~G_{M}~A_{F}~G_{M}
P-U_{M}~U_{F3}~G_{M}A_{L}~U_{M3}C_{L4}~U_{M3}A_{F}~A_{L}A_{F}~U_{M3}G_{F}~C_{L4}A_{F}~U_{M3}~U_{L3}~G_{M}~U_{L3}~G_{M}~A_{F}~G_{M}
P-U_{M}~U_{F12}~G_{M}A_{L}~U_{M12}C_{L4}~U_{M}A_{F}~A_{L}A_{F}~U_{M}G_{F}~C_{L4}A_{F}~U_{M12}~U_{L}~G_{M}~U_{L12}~G_{M}~A_{F}~G_{M}
P-U_{M}~U_{F}~G_{M}A_{L}~U_{M12}C_{L}~U_{M12}A_{F}~A_{L}A_{F}~U_{M}G_{F}~C_{L}A_{F}~U_{M12}~U_{L}~G_{M}~U_{L12}~G_{M}~A_{F}~G_{M}
P-U_{M}~U_{F}~G_{M}A_{L}~U_{M12}C_{L}~U_{M}A_{F}~A_{L}A_{F}~U_{M12}G_{F}~C_{L}A_{F}~U_{M12}~U_{L}~G_{M}~U_{L12}~G_{M}~A_{F}~G_{M}
P-U_{M}~U_{F}~G_{M}A_{L}~U_{M12}C_{L}~U_{M}A_{F}~A_{L}A_{F}~U_{M}G_{F}~C_{L}A_{F}~U_{M12}~U_{L12}~G_{M}~U_{L12}~G_{M}~A_{F}~G_{M}
P-U_{M}~U_{F12}~G_{M}A_{L}~U_{M}C_{L4}~U_{M}A_{F}~A_{L}A_{F}~U_{M}G_{F}~C_{L4}A_{F}~U_{M}~U_{L}~G_{M}~U_{L12}~G_{M}~A_{F}~G_{M}
P-U_{M}~U_{F12}~G_{M}A_{L}~U_{M12}C_{L4}~U_{M12}A_{F}~A_{L}A_{F}~U_{M12}G_{F}~C_{L4}A_{F}~U_{M12}~U_{L12}~G_{M}~U_{L12}~G_{M}~A_{F}~G_{M}
P-U_{M}~U_{F18}~G_{M}A_{L}~U_{M18}C_{L4}~U_{M}A_{F}~A_{L}A_{F}~U_{M}G_{F}~C_{L4}A_{F}~U_{M18}~U_{L}~G_{M}~U_{L12}~G_{M}~A_{F}~G_{M}
P-U_{M}~U_{F}~G_{M}A_{L}~U_{M18}C_{L}~U_{M18}A_{F}~A_{L}A_{F}~U_{M}G_{F}~C_{L}A_{F}~U_{M18}~U_{L}~G_{M}~U_{L18}~G_{M}~A_{F}~G_{M}
P-U_{M}~U_{F}~G_{M}A_{L}~U_{M18}C_{L}~U_{M}A_{F}~A_{L}A_{F}~U_{M18}G_{F}~C_{L}A_{F}~U_{M18}~U_{L}~G_{M}~U_{L18}~G_{M}~A_{F}~G_{M}
P-U_{M}~U_{F}~G_{M}A_{L}~U_{M18}C_{L}~U_{M}A_{F}~A_{L}A_{F}~U_{M}G_{F}~C_{L}A_{F}~U_{M18}~U_{L18}~G_{M}~U_{L18}~G_{M}~A_{F}~G_{M}
P-U_{M}~U_{F18}~G_{M}A_{L}~U_{M}C_{L4}~U_{M}A_{F}~A_{L}A_{F}~U_{M}G_{F}~C_{L4}A_{F}~U_{M}~U_{L}~G_{M}~U_{L18}~G_{M}~A_{F}~G_{M}
P-U_{M}~U_{F18}~G_{M}A_{L}~U_{M18}C_{L4}~U_{M18}A_{F}~A_{L}A_{F}~U_{M18}G_{F}~C_{L4}A_{F}~U_{M18}~U_{L18}~G_{M}~U_{L18}~G_{M}~A_{F}~G_{M}
P-U_{M}~U_{F3}~G_{M}A_{L}~U_{M3}C_{L4}~U_{M}A_{F}~A_{L}A_{F}~U_{M}G_{F}~C_{L4}A~U_{M3}~U_{L}~G_{M}~U_{L3}~G_{M}~A_{F}~G_{M}
P-U_{M}~U_{F}~G_{M}A_{L}~U_{M3}C_{L}~U_{M3}A_{F}~A_{L}A_{F}~U_{M}G_{F}~C_{L}A~U_{M3}~U_{L}~G_{M}~U_{L3}~G_{M}~A_{F}~G_{M}
P-U_{M}~U_{F}~G_{M}A_{L}~U_{M3}C_{L}~U_{M}A_{F}~A_{L}A_{F}~U_{M3}G_{F}~C_{L}A~U_{M3}~U_{L}~G_{M}~U_{L3}~G_{M}~A_{F}~G_{M}
P-U_{M}~U_{F}~G_{M}A_{L}~U_{M3}C_{L}~U_{M}A_{F}~A_{L}A_{F}~U_{M}G_{F}~C_{L}A~U_{M3}~U_{L3}~G_{M}~U_{L3}~G_{M}~A_{F}~G_{M}
P-U_{M}~U_{F3}~G_{M}A_{L}~U_{M}C_{L4}~U_{M}A_{F}~A_{L}A_{F}~U_{M}G_{F}~C_{L4}A~U_{M}~U_{L}~G_{M}~U_{L3}~G_{M}~A_{F}~G_{M}
P-U_{M}~U_{F3}~G_{M}A_{L}~U_{M3}C_{L4}~U_{M3}A_{F}~A_{L}A_{F}~U_{M3}G_{F}~C_{L4}A~U_{M3}~U_{L3}~G_{M}~U_{L3}~G_{M}~A_{F}~G_{M}
P-U_{M}~U_{F12}~G_{M}A_{L}~U_{M12}C_{L4}~U_{M}A_{F}~A_{L}A_{F}~U_{M}G_{F}~C_{L4}A~U_{M12}~U_{L}~G_{M}~U_{L12}~G_{M}~A_{F}~G_{M}
P-U_{M}~U_{F}~G_{M}A_{L}~U_{M12}C_{L}~U_{M12}A_{F}~A_{L}A_{F}~U_{M}G_{F}~C_{L}A~U_{M12}~U_{L}~G_{M}~U_{L12}~G_{M}~A_{F}~G_{M}
P-U_{M}~U_{F}~G_{M}A_{L}~U_{M12}C_{L}~U_{M}A_{F}~A_{L}A_{F}~U_{M12}G_{F}~C_{L}A~U_{M12}~U_{L}~G_{M}~U_{L12}~G_{M}~A_{F}~G_{M}
P-U_{M}~U_{F}~G_{M}A_{L}~U_{M12}C_{L}~U_{M}A_{F}~A_{L}A_{F}~U_{M}G_{F}~C_{L}A~U_{M12}~U_{L12}~G_{M}~U_{L12}~G_{M}~A_{F}~G_{M}
P-U_{M}~U_{F12}~G_{M}A_{L}~U_{M}C_{L4}~U_{M}A_{F}~A_{L}A_{F}~U_{M}G_{F}~C_{L4}A~U_{M}~U_{L}~G_{M}~U_{L12}~G_{M}~A_{F}~G_{M}
P-U_{M}~U_{F12}~G_{M}A_{L}~U_{M12}C_{L4}~U_{M12}A_{F}~A_{L}A_{F}~U_{M12}G_{F}~C_{L4}A~U_{M12}~U_{L12}~G_{M}~U_{L12}~G_{M}~A_{F}~G_{M}
P-U_{M}~U_{F18}~G_{M}A_{L}~U_{M18}C_{L4}~U_{M}A_{F}~A_{L}A_{F}~U_{M}G_{F}~C_{L4}A~U_{M18}~U_{L}~G_{M}~U_{L18}~G_{M}~A_{F}~G_{M}
P-U_{M}~U_{F}~G_{M}A_{L}~U_{M18}C_{L}~U_{M18}A_{F}~A_{L}A_{F}~U_{M}G_{F}~C_{L}A~U_{M18}~U_{L}~G_{M}~U_{L18}~G_{M}~A_{F}~G_{M}
P-U_{M}~U_{F}~G_{M}A_{L}~U_{M18}C_{L}~U_{M}A_{F}~A_{L}A_{F}~U_{M18}G_{F}~C_{L}A~U_{M18}~U_{L}~G_{M}~U_{L18}~G_{M}~A_{F}~G_{M}
P-U_{M}~U_{F}~G_{M}A_{L}~U_{M18}C_{L}~U_{M}A_{F}~A_{L}A_{F}~U_{M}G_{F}~C_{L}A~U_{M18}~U_{L18}~G_{M}~U_{L18}~G_{M}~A_{F}~G_{M}
P-U_{M}~U_{F18}~G_{M}A_{L}~U_{M}C_{L4}~U_{M}A_{F}~A_{L}A_{F}~U_{M}G_{F}~C_{L4}A~U_{M}~U_{L}~G_{M}~U_{L18}~G_{M}~A_{F}~G_{M}
P-U_{M}~U_{F18}~G_{M}A_{L}~U_{M18}C_{L4}~U_{M18}A_{F}~A_{L}A_{F}~U_{M18}G_{F}~C_{L4}A~U_{M18}~U_{L18}~G_{M}~U_{L18}~G_{M}~A_{F}~G_{M}
P-U_{M}~U_{F}~G_{M}A_{L}~U_{M}C_{F}~U_{M}A_{L}~A_{M}A_{L}~U_{M}G_{L}~C_{M}A_{F}~U_{L}~U_{F}~G_{L}~U_{F}~G_{M}
P-U_{M}~U_{F}~G_{M}A_{L}~U_{M}C_{F}~U_{M}A_{L}~A_{M}A_{L}~U_{M}G_{L}~C_{M}A~U_{L}~U_{F}~G_{L}~U_{F}~G_{M}
P-U_{M}~U_{F3}~G_{M}A_{L}~U_{M3}C_{F4}~U_{M}A_{L}~A_{M}A_{L}~U_{M}G_{L}~C_{M4}A_{F}~U_{L3}~U_{F}~G_{L}~U_{F3}~G_{M}~A_{F}~G_{M}
P-U_{M}~U_{F}~G_{M}A_{L}~U_{M3}C_{F}~U_{M3}A_{L}~A_{M}A_{L}~U_{M}G_{L}~C_{M}A_{F}~U_{L3}~U_{F}~G_{L}~U_{F3}~G_{M}~A_{F}~G_{M}
P-U_{M}~U_{F}~G_{M}A_{L}~U_{M3}C_{F}~U_{M}A_{L}~A_{M}A_{L}~U_{M3}G_{L}~C_{M}A_{F}~U_{L3}~U_{F}~G_{L}~U_{F3}~G_{M}~A_{F}~G_{M}
P-U_{M}~U_{F}~G_{M}A_{L}~U_{M3}C_{F}~U_{M}A_{L}~A_{M}A_{L}~U_{M}G_{L}~C_{M}A_{F}~U_{L3}~U_{F3}~G_{L}~U_{F3}~G_{M}~A_{F}~G_{M}
P-U_{M}~U_{F3}~G_{M}A_{L}~U_{M}C_{F4}~U_{M}A_{L}~A_{M}A_{L}~U_{M}G_{L}~C_{M4}A_{F}~U_{L}~U_{F}~G_{L}~U_{F3}~G_{M}~A_{F}~G_{M}
P-U_{M}~U_{F3}~G_{M}A_{L}~U_{M3}C_{F4}~U_{M3}A_{L}~A_{M}A_{L}~U_{M3}G_{L}~C_{M4}A_{F}~U_{L3}~U_{F3}~G_{L}~U_{F3}~G_{M}~A_{F}~G_{M}
P-U_{M}~U_{F12}~G_{M}A_{L}~U_{M12}C_{F4}~U_{M}A_{L}~A_{M}A_{L}~U_{M}G_{L}~C_{M4}A_{F}~U_{L12}~U_{F}~G_{L}~U_{F12}~G_{M}~A_{F}~G_{M}
P-U_{M}~U_{F}~G_{M}A_{L}~U_{M12}C_{F}~U_{M12}A_{L}~A_{M}A_{L}~U_{M}G_{L}~C_{M}A_{F}~U_{L12}~U_{F}~G_{L}~U_{F12}~G_{M}~A_{F}~G_{M}
P-U_{M}~U_{F}~G_{M}A_{L}~U_{M12}C_{F}~U_{M}A_{L}~A_{M}A_{L}~U_{M12}G_{L}~C_{M}A_{F}~U_{L12}~U_{F}~G_{L}~U_{F12}~G_{M}~A_{F}~G_{M}
P-U_{M}~U_{F}~G_{M}A_{L}~U_{M12}C_{F}~U_{M}A_{L}~A_{M}A_{L}~U_{M}G_{L}~C_{M}A_{F}~U_{L12}~U_{F12}~G_{L}~U_{F12}~G_{M}~A_{F}~G_{M}
P-U_{M}~U_{F12}~G_{M}A_{F}~U_{M}C_{F4}~U_{M}A_{L}~A_{M}A_{L}~U_{M}G_{L}~C_{M4}A_{F}~U_{L}~U_{F}~G_{L}~U_{F12}~G_{M}~A_{F}~G_{M}
P-U_{M}~U_{F12}~G_{M}A_{L}~U_{M12}C_{F4}~U_{M12}A_{L}~A_{M}A_{L}~U_{M12}G_{L}~C_{M4}A_{F}~U_{L12}~U_{F12}~G_{L}~U_{F12}~G_{M}~A_{F}~G_{M}
P-U_{M}~U_{F18}~G_{M}A_{L}~U_{M18}C_{F4}~U_{M}A_{L}~A_{M}A_{L}~U_{M}G_{L}~C_{M4}A_{F}~U_{L18}~U_{F}~G_{L}~U_{F18}~G_{M}~A_{F}~G_{M}
P-U_{M}~U_{F}~G_{M}A_{L}~U_{M18}C_{F}~U_{M18}A_{L}~A_{M}A_{L}~U_{M}G_{L}~C_{M}A_{F}~U_{L18}~U_{F}~G_{L}~U_{F18}~G_{M}~A_{F}~G_{M}
P-U_{M}~U_{F}~G_{M}A_{L}~U_{M18}C_{F}~U_{M}A_{L}~A_{M}A_{L}~U_{M18}G_{L}~C_{M}A_{F}~U_{L18}~U_{F}~G_{L}~U_{F18}~G_{M}~A_{F}~G_{M}
P-U_{M}~U_{F}~G_{M}A_{L}~U_{M18}C_{F}~U_{M}A_{L}~A_{M}A_{L}~U_{M}G_{L}~C_{M}A_{F}~U_{L18}~U_{F18}~G_{L}~U_{F18}~G_{M}~A_{F}~G_{M}
P-U_{M}~U_{F18}~G_{M}A_{L}~U_{M}C_{F4}~U_{M}A_{L}~A_{M}A_{L}~U_{M}G_{L}~C_{M4}A_{F}~U_{L}~U_{F}~G_{L}~U_{F18}~G_{M}~A_{F}~G_{M}
P-U_{M}~U_{F18}~G_{M}A_{L}~U_{M18}C_{F4}~U_{M18}A_{L}~A_{M}A_{L}~U_{M18}G_{L}~C_{M4}A_{F}~U_{L18}~U_{F18}~G_{L}~U_{F18}~G_{M}~A_{F}~G_{M}
P-U_{M}~U_{F3}~G_{M}A_{L}~U_{M3}C_{F4}~U_{M}A_{L}~A_{M}A_{L}~U_{M}G_{L}~C_{M4}A~U_{L3}~U_{F}~G_{L}~U_{F3}~G_{M}~A_{F}~G_{M}
P-U_{M}~U_{F}~G_{M}A_{L}~U_{M3}C_{F}~U_{M3}A_{L}~A_{M}A_{L}~U_{M}G_{L}~C_{M}A~U_{L3}~U_{F}~G_{L}~U_{F3}~G_{M}~A_{F}~G_{M}
P-U_{M}~U_{F}~G_{M}A_{L}~U_{M3}C_{F}~U_{M}A_{L}~A_{M}A_{L}~U_{M3}G_{L}~C_{M}A~U_{L3}~U_{F}~G_{L}~U_{F3}~G_{M}~A_{F}~G_{M}
P-U_{M}~U_{F}~G_{M}A_{L}~U_{M3}C_{F}~U_{M}A_{L}~A_{M}A_{L}~U_{M}G_{L}~C_{M}A~U_{L3}~U_{F3}~G_{L}~U_{F3}~G_{M}~A_{F}~G_{M}
P-U_{M}~U_{F3}~G_{M}A_{L}~U_{M}C_{F4}~U_{M}A_{L}~A_{M}A_{L}~U_{M}G_{L}~C_{M4}A~U_{L}~U_{F}~G_{L}~U_{F3}~G_{M}~A_{F}~G_{M}
P-U_{M}~U_{F3}~G_{M}A_{L}~U_{M3}C_{F4}~U_{M3}A_{L}~A_{M}A_{L}~U_{M3}G_{L}~C_{M4}A~U_{L3}~U_{F3}~G_{L}~U_{F3}~G_{M}~A_{F}~G_{M}
P-U_{M}~U_{F12}~G_{M}A_{L}~U_{M12}C_{F4}~U_{M}A_{L}~A_{M}A_{L}~U_{M}G_{L}~C_{M4}A~U_{L12}~U_{F}~G_{L}~U_{F12}~G_{M}~A_{F}~G_{M}
P-U_{M}~U_{F}~G_{M}A_{L}~U_{M12}C_{F}~U_{M12}A_{L}~A_{M}A_{L}~U_{M}G_{L}~C_{M}A~U_{L12}~U_{F}~G_{L}~U_{F12}~G_{M}~A_{F}~G_{M}
P-U_{M}~U_{F}~G_{M}A_{L}~U_{M12}C_{F}~U_{M}A_{L}~A_{M}A_{L}~U_{M12}G_{L}~C_{M}A~U_{L12}~U_{F}~G_{L}~U_{F12}~G_{M}~A_{F}~G_{M}
P-U_{M}~U_{F}~G_{M}A_{L}~U_{M12}C_{F}~U_{M}A_{L}~A_{M}A_{L}~U_{M}G_{L}~C_{M}A~U_{L12}~U_{F12}~G_{L}~U_{F12}~G_{M}~A_{F}~G_{M}
P-U_{M}~U_{F12}~G_{M}A_{L}~U_{M}C_{F4}~U_{M}A_{L}~A_{M}A_{L}~U_{M}G_{L}~C_{M4}A~U_{L}~U_{F}~G_{L}~U_{F12}~G_{M}~A_{F}~G_{M}
P-U_{M}~U_{F12}~G_{M}A_{L}~U_{M12}C_{F4}~U_{M12}A_{L}~A_{M}A_{L}~U_{M12}G_{L}~C_{M4}A~U_{L12}~U_{F12}~G_{L}~U_{F12}~G_{M}~A_{F}~G_{M}
P-U_{M}~U_{F18}~G_{M}A_{L}~U_{M18}C_{F4}~U_{M}A_{L}~A_{M}A_{L}~U_{M}G_{L}~C_{M4}A~U_{L18}~U_{F}~G_{L}~U_{F18}~G_{M}~A_{F}~G_{M}
P-U_{M}~U_{F}~G_{M}A_{L}~U_{M18}C_{F}~U_{M18}A_{L}~A_{M}A_{L}~U_{M}G_{L}~C_{M}A~U_{L18}~U_{F}~G_{L}~U_{F18}~G_{M}~A_{F}~G_{M}
P-U_{M}~U_{F}~G_{M}A_{L}~U_{M18}C_{F}~U_{M}A_{L}~A_{M}A_{L}~U_{M18}G_{L}~C_{M}A~U_{L18}~U_{F}~G_{L}~U_{F18}~G_{M}~A_{F}~G_{M}
P-U_{M}~U_{F}~G_{M}A_{L}~U_{M18}C_{F}~U_{M}A_{L}~A_{M}A_{L}~U_{M}G_{L}~C_{M}A~U_{L18}~U_{F18}~G_{L}~U_{F18}~G_{M}~A_{F}~G_{M}
P-U_{M}~U_{F18}~G_{M}A_{L}~U_{M}C_{F4}~U_{M}A_{L}~A_{M}A_{L}~U_{M}G_{L}~C_{M4}A~U_{L}~U_{F}~G_{L}~U_{F18}~G_{M}~A_{F}~G_{M}
P-U_{M}~U_{F18}~G_{M}A_{L}~U_{M18}C_{F4}~U_{M18}A_{L}~A_{M}A_{L}~U_{M18}G_{L}~C_{M4}A~U_{L18}~U_{F18}~G_{L}~U_{F18}~G_{M}~A_{F}~G_{M}

### Table 16

### Examples of Murine Apo-B ags-siRNA Compounds

UUAAG_{F}GAAGCCUUACUGG

Haringsma et al. (Nucleic Acids Res 40: 4125-36, 2012) used this sequence to generate ssRNAi compounds against murine APO-B target site 8786 including the following:

P-U_{F}U_{F}A_{F}A_{F}G_{F}A_{F}G_{F}A_{F}A_{F}G_{F}C_{F}C_{F}U_{F}U_{F}A_{F}C_{F}U_{F}G_{F}G_{F}U_{M}U_{M}

Any of the 5′-end nucleosides (P-U_{F} or P-U_{M}) shown in the examples of agsRNAi compounds in Tables 16A and 16B can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-Tₘₒₑ, 5′-VP-T_{E}, 5′-VP-T_{Q} and 5′-VP-T_{B}.

Any of the nucleosides in Tables 16A and 16B with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the nucleosides in Tables 16A and 16B with an L modification found in the overhang precursor can have the L replaced with any of those units and linkages provided for herein for overhang precursors including one or more of the sugar or sugar analogs independently selected from the group consisting of: F, J, W, V, Y, T, TL and I.

Any of the compounds in Tables 16A and 16B can any of those units and linkages provided for herein for overhang precursors.

B. Examples of Application of AGSD to this Compound:

TABLE 16A
Compositions of Matter Suitable for Use in vitro
or in a Subject with a Protective Carrier (5′-3′)

P-U_{F}U_{F3}A_{F}A_{F}G_{F}A_{F}G_{F}A_{F}A_{F}G_{F}C_{F}C_{F}U_{F3}U_{F}A_{F}C_{F}U_{F3}G_{F}G_{F}C_{L}G_{L}C_{F}
P-U_{F}U_{F3}A_{F}A_{F}G_{F}A_{F}G_{F}A_{F}A_{F}G_{F}C_{F}C_{F4}U_{F3}U_{F}A_{F}C_{F}U_{F3}G_{F}G_{F}C_{L}G_{L}C_{F}
P-U_{F}U_{F3}A_{F}A_{F}G_{F}A_{F}G_{F}A_{F}A_{F}G_{F}C_{F}C_{F4}U_{F3}U_{F}A_{F}C_{F4}U_{F3}G_{F}G_{F}C_{L}G_{L}C_{F}
P-U_{F}U_{F3}A_{F}A_{F}G_{F}A_{F}G_{F}A_{F}A_{F}G_{F}C_{F4}C_{F4}U_{F3}U_{F}A_{F}C_{F4}U_{F3}G_{F}G_{F}C_{L}G_{L}C_{F}
P-U_{F}U_{F3}A_{F}A_{F2}G_{F}A_{F}G_{F}A_{F}A_{F}G_{F}C_{F4}C_{F4}U_{F3}U_{F}A_{F}C_{F4}U_{F3}G_{F}G_{F}C_{L}G_{L}C_{F}
P-U_{F}U_{F12}A_{F}A_{F}G_{F}A_{F}G_{F}A_{F}A_{F}G_{F}C_{F}C_{F}U_{F12}U_{F}A_{F}C_{F}U_{F12}G_{F}G_{F}C_{L}G_{L}C_{F}
P-U_{F}U_{F12}A_{F}A_{F}G_{F}A_{F}G_{F}A_{F}A_{F}G_{F}C_{F}C_{F4}U_{F12}U_{F}A_{F}C_{F}U_{F12}G_{F}G_{F}C_{L}G_{L}C_{F}
P-U_{F}U_{F12}A_{F}A_{F}G_{F}A_{F}G_{F}A_{F}A_{F}G_{F}C_{F}C_{F4}U_{F12}U_{F}A_{F}C_{F4}U_{F12}G_{F}G_{F}C_{L}G_{L}C_{F}
P-U_{F}U_{F12}A_{F}A_{F}G_{F}A_{F}G_{F}A_{F}A_{F}G_{F}C_{F4}C_{F4}U_{F12}U_{F}A_{F}C_{F4}U_{F12}G_{F}G_{F}C_{L}G_{L}C_{F}
P-U_{F}U_{F12}A_{F}A_{F2}G_{F}A_{F}G_{F}A_{F}A_{F}G_{F}C_{F4}C_{F4}U_{F12}U_{F}A_{F}C_{F4}U_{F12}G_{F}G_{F}C_{L}G_{L}C_{F}
P-U_{F}U_{F18}A_{F}A_{F}G_{F}A_{F}G_{F}A_{F}A_{F}G_{F}C_{F}C_{F}U_{F18}U_{F}A_{F}C_{F}U_{F18}G_{F}G_{F}C_{L}G_{L}C_{F}
P-U_{F}U_{F18}A_{F}A_{F}G_{F}A_{F}G_{F}A_{F}A_{F}G_{F}C_{F}C_{F4}U_{F18}U_{F}A_{F}C_{F}U_{F18}G_{F}G_{F}C_{L}G_{L}C_{F}
P-U_{F}U_{F18}A_{F}A_{F}G_{F}A_{F}G_{F}A_{F}A_{F}G_{F}C_{F}C_{F4}U_{F18}U_{F}A_{F}C_{F4}U_{F18}G_{F}G_{F}C_{L}G_{L}C_{F}
P-U_{F}U_{F18}A_{F}A_{F}G_{F}A_{F}G_{F}A_{F}A_{F}G_{F}C_{F4}C_{F4}U_{F18}U_{F}A_{F}C_{F4}U_{F18}G_{F}G_{F}C_{L}G_{L}C_{F}
P-U_{F}U_{F18}A_{F}A_{F2}G_{F}A_{F}G_{F}A_{F}A_{F}G_{F}C_{F4}C_{F4}U_{F18}U_{F}A_{F}C_{F4}U_{F18}G_{F}G_{F}C_{L}G_{L}C_{F}
P-U_{F}U_{F3}A_{F}A_{F}G_{F}A_{F}G_{F}A_{F}A_{F}G_{F}C_{F}C_{F}U_{F3}UA_{F}C_{F}U_{F3}G_{F}G_{F}C_{L}G_{L}C_{F}
P-U_{F}U_{F3}A_{F}A_{F}G_{F}A_{F}G_{F}A_{F}A_{F}G_{F}C_{F}C_{F4}U_{F3}UA_{F}C_{F}U_{F3}G_{F}G_{F}C_{L}G_{L}C_{F}
P-U_{F}U_{F3}A_{F}A_{F}G_{F}A_{F}G_{F}A_{F}A_{F}G_{F}C_{F}C_{F4}U_{F3}UA_{F}C_{F4}U_{F3}G_{F}G_{F}C_{L}G_{L}C_{F}
P-U_{F}U_{F3}A_{F}A_{F}G_{F}A_{F}G_{F}A_{F}A_{F}G_{F}C_{F4}C_{F4}U_{F3}UA_{F}C_{F4}U_{F3}G_{F}G_{F}C_{L}G_{L}C_{F}
P-U_{F}U_{F3}A_{F}A_{F2}G_{F}A_{F}G_{F}A_{F}A_{F}G_{F}C_{F4}C_{F4}U_{F3}UA_{F}C_{F4}U_{F3}G_{F}G_{F}C_{L}G_{L}C_{F}
P-U_{F}U_{F12}A_{F}A_{F}G_{F}A_{F}G_{F}A_{F}A_{F}G_{F}C_{F}C_{F}U_{F12}UA_{F}C_{F}U_{F12}G_{F}G_{F}C_{L}G_{L}C_{F}
P-U_{F}U_{F12}A_{F}A_{F}G_{F}A_{F}G_{F}A_{F}A_{F}G_{F}C_{F}C_{F4}U_{F12}UA_{F}C_{F}U_{F12}G_{F}G_{F}C_{L}G_{L}C_{F}
P-U_{F}U_{F12}A_{F}A_{F}G_{F}A_{F}G_{F}A_{F}A_{F}G_{F}C_{F}C_{F4}U_{F12}UA_{F}C_{F4}U_{F12}G_{F}G_{F}C_{L}G_{L}C_{F}
P-U_{F}U_{F12}A_{F}A_{F}G_{F}A_{F}G_{F}A_{F}A_{F}G_{F}C_{F4}C_{F4}U_{F12}UA_{F}C_{F4}U_{F12}G_{F}G_{F}C_{L}G_{L}C_{F}
P-U_{F}U_{F12}A_{F}A_{F2}G_{F}A_{F}G_{F}A_{F}A_{F}G_{F}C_{F4}C_{F4}U_{F12}UA_{F}C_{F4}U_{F12}G_{F}G_{F}C_{L}G_{L}C_{F}
P-U_{F}U_{F18}A_{F}A_{F}G_{F}A_{F}G_{F}A_{F}A_{F}G_{F}C_{F}C_{F}U_{F18}UA_{F}C_{F}U_{F18}G_{F}G_{F}C_{L}G_{L}C_{F}
P-U_{F}U_{F18}A_{F}A_{F}G_{F}A_{F}G_{F}A_{F}A_{F}G_{F}C_{F}C_{F4}U_{F18}UA_{F}C_{F}U_{F18}G_{F}G_{F}C_{L}G_{L}C_{F}
P-U_{F}U_{F18}A_{F}A_{F}G_{F}A_{F}G_{F}A_{F}A_{F}G_{F}C_{F}C_{F4}U_{F18}UA_{F}C_{F4}U_{F18}G_{F}G_{F}C_{L}G_{L}C_{F}
P-U_{F}U_{F18}A_{F}A_{F}G_{F}A_{F}G_{F}A_{F}A_{F}G_{F}C_{F4}C_{F4}U_{F18}UA_{F}C_{F4}U_{F18}G_{F}G_{F}C_{L}G_{L}C_{F}
P-U_{F}U_{F18}A_{F}A_{F2}G_{F}A_{F}G_{F}A_{F}A_{F}G_{F}C_{F4}C_{F4}U_{F18}UA_{F}C_{F4}U_{F18}G_{F}G_{F}C_{L}G_{L}C_{F}
P-U_{F}U_{F}A_{L}A_{F}G_{F}A_{L}G_{F}A_{F}A_{L}G_{F}C_{F}C_{F}U_{F}U_{F}A_{L}C_{F}U_{F}G_{L}G_{F}C_{L}G_{L}C_{F}
P-U_{F}U_{F}A_{L}A_{F}G_{F}A_{L}G_{F}A_{F}A_{L}G_{F}C_{F}C_{F}U_{F}UA_{L}C_{F}U_{F}G_{L}G_{F}C_{L}G_{L}C_{F}
P-U_{F}U_{F3}A_{L}A_{F}G_{F}A_{L}G_{F}A_{F}A_{L}G_{F}C_{F}C_{F}U_{F3}U_{F}A_{L}C_{F}U_{F3}G_{L}G_{F}C_{L}G_{L}C_{F}
P-U_{F}U_{F3}A_{L}A_{F}G_{F}A_{L}G_{F}A_{F}A_{L}G_{F}C_{F}C_{F4}U_{F3}U_{F}A_{L}C_{F}U_{F3}G_{L}G_{F}C_{L}G_{L}C_{F}
P-U_{F}U_{F3}A_{L}A_{F}G_{F}A_{L}G_{F}A_{F}A_{L}G_{F}C_{F}C_{F4}U_{F3}U_{F}A_{L}C_{F4}U_{F3}G_{L}G_{F}C_{L}G_{L}C_{F}
P-U_{F}U_{F3}A_{L}A_{F}G_{F}A_{L}G_{F}A_{F}A_{L}G_{F}C_{F4}C_{F4}U_{F3}U_{F}A_{L}C_{F4}U_{F3}G_{L}G_{F}C_{L}G_{L}C_{F}
P-U_{F}U_{F3}A_{L}A_{F2}G_{F}A_{L}G_{F}A_{F}A_{L}G_{F}C_{F4}C_{F4}U_{F3}U_{F}A_{L}C_{F4}U_{F3}G_{L}G_{F}C_{L}G_{L}C_{F}
P-U_{F}U_{F12}A_{L}A_{F}G_{F}A_{L}G_{F}A_{F}A_{L}G_{F}C_{F}C_{F}U_{F12}U_{F}A_{L}C_{F}U_{F12}G_{L}G_{F}C_{L}G_{L}C_{F}
P-U_{F}U_{F12}A_{L}A_{F}G_{F}A_{L}G_{F}A_{F}A_{L}G_{F}C_{F}C_{F4}U_{F12}U_{F}A_{L}C_{F}U_{F12}G_{L}G_{F}C_{L}G_{L}C_{F}
P-U_{F}U_{F12}A_{L}A_{F}G_{F}A_{L}G_{F}A_{F}A_{L}G_{F}C_{F}C_{F4}U_{F12}U_{F}A_{L}C_{F4}U_{F12}G_{L}G_{F}C_{L}G_{L}C_{F}
P-U_{F}U_{F12}A_{L}A_{F}G_{F}A_{L}G_{F}A_{F}A_{L}G_{F}C_{F4}C_{F4}U_{F12}U_{F}A_{L}C_{F4}U_{F12}G_{L}G_{F}C_{L}G_{L}C_{F}
P-U_{F}U_{F12}A_{L}A_{F2}G_{F}A_{L}G_{F}A_{F}A_{L}G_{F}C_{F4}C_{F4}U_{F12}U_{F}A_{L}C_{F4}U_{F12}G_{L}G_{F}C_{L}G_{L}C_{F}
P-U_{F}U_{F18}A_{L}A_{F}G_{F}A_{L}G_{F}A_{F}A_{L}G_{F}C_{F}C_{F}U_{F18}U_{F}A_{L}C_{F}U_{F18}G_{L}G_{F}C_{L}G_{L}C_{F}
P-U_{F}U_{F18}A_{L}A_{F}G_{F}A_{L}G_{F}A_{F}A_{L}G_{F}C_{F}C_{F4}U_{F18}U_{F}A_{L}C_{F}U_{F18}G_{L}G_{F}C_{L}G_{L}C_{F}
P-U_{F}U_{F18}A_{L}A_{F}G_{F}A_{L}G_{F}A_{F}A_{L}G_{F}C_{F}C_{F4}U_{F18}U_{F}A_{L}C_{F4}U_{F18}G_{L}G_{F}C_{L}G_{L}C_{F}
P-U_{F}U_{F18}A_{L}A_{F}G_{F}A_{L}G_{F}A_{F}A_{L}G_{F}C_{F4}C_{F4}U_{F18}U_{F}A_{L}C_{F4}U_{F18}G_{L}G_{F}C_{L}G_{L}C_{F}
P-U_{F}U_{F18}A_{L}A_{F2}G_{F}A_{L}G_{F}A_{F}A_{L}G_{F}C_{F4}C_{F4}U_{F18}U_{F}A_{L}C_{F4}U_{F18}G_{L}G_{F}C_{L}G_{L}C_{F}
P-U_{F}U_{F3}A_{L}A_{F}G_{F}A_{L}G_{F}A_{F}A_{L}G_{F}C_{F}C_{F}U_{F3}UA_{L}C_{F}U_{F3}G_{L}G_{F}C_{L}G_{L}C_{F}
P-U_{F}U_{F3}A_{L}A_{F}G_{F}A_{L}G_{F}A_{F}A_{L}G_{F}C_{F}C_{F4}U_{F3}UA_{L}C_{F}U_{F3}G_{L}G_{F}C_{L}G_{L}C_{F}
P-U_{F}U_{F3}A_{L}A_{F}G_{F}A_{L}G_{F}A_{F}A_{L}G_{F}C_{F}C_{F4}U_{F3}UA_{L}C_{F4}U_{F3}G_{L}G_{F}C_{L}G_{L}C_{F}
P-U_{F}U_{F3}A_{L}A_{F}G_{F}A_{L}G_{F}A_{F}A_{L}G_{F}C_{F4}C_{F4}U_{F3}UA_{L}C_{F4}U_{F3}G_{L}G_{F}C_{L}G_{L}C_{F}
P-U_{F}U_{F3}A_{L}A_{F2}G_{F}A_{L}G_{F}A_{F}A_{L}G_{F}C_{F4}C_{F4}U_{F3}UA_{L}C_{F4}U_{F3}G_{L}G_{F}C_{L}G_{L}C_{F}
P-U_{F}U_{F12}A_{L}A_{F}G_{F}A_{L}G_{F}A_{F}A_{L}G_{F}C_{F}C_{F}U_{F12}UA_{L}C_{F}U_{F12}G_{L}G_{F}C_{L}G_{L}C_{F}
P-U_{F}U_{F12}A_{L}A_{F}G_{F}A_{L}G_{F}A_{F}A_{L}G_{F}C_{F}C_{F4}U_{F12}UA_{L}C_{F}U_{F12}G_{L}G_{F}C_{L}G_{L}C_{F}
P-U_{F}U_{F12}A_{L}A_{F}G_{F}A_{L}G_{F}A_{F}A_{L}G_{F}C_{F}C_{F4}U_{F12}UA_{L}C_{F4}U_{F12}G_{L}G_{F}C_{L}G_{L}C_{F}
P-U_{F}U_{F12}A_{L}A_{F}G_{F}A_{L}G_{F}A_{F}A_{L}G_{F}C_{F4}C_{F4}U_{F12}UA_{L}C_{F4}U_{F12}G_{L}G_{F}C_{L}G_{L}C_{F}
P-U_{F}U_{F12}A_{L}A_{F2}G_{F}A_{L}G_{F}A_{F}A_{L}G_{F}C_{F4}C_{F4}U_{F12}UA_{L}C_{F4}U_{F12}G_{L}G_{F}C_{L}G_{L}C_{F}
P-U_{F}U_{F18}A_{L}A_{F}G_{F}A_{L}G_{F}A_{F}A_{L}G_{F}C_{F}C_{F}U_{F18}UA_{L}C_{F}U_{F18}G_{L}G_{F}C_{L}G_{L}C_{F}
P-U_{F}U_{F18}A_{L}A_{F}G_{F}A_{L}G_{F}A_{F}A_{L}G_{F}C_{F}C_{F4}U_{F18}UA_{L}C_{F}U_{F18}G_{L}G_{F}C_{L}G_{L}C_{F}
P-U_{F}U_{F18}A_{L}A_{F}G_{F}A_{L}G_{F}A_{F}A_{L}G_{F}C_{F}C_{F4}U_{F18}UA_{L}C_{F4}U_{F18}G_{L}G_{F}C_{L}G_{L}C_{F}
P-U_{F}U_{F18}A_{L}A_{F}G_{F}A_{L}G_{F}A_{F}A_{L}G_{F}C_{F4}C_{F4}U_{F18}UA_{L}C_{F4}U_{F18}G_{L}G_{F}C_{L}G_{L}C_{F}
P-U_{F}U_{F18}A_{L}A_{F2}G_{F}A_{L}G_{F}A_{F}A_{L}G_{F}C_{F4}C_{F4}U_{F18}UA_{L}C_{F4}U_{F18}G_{L}G_{F}C_{L}G_{L}C_{F}
P-U_{F}U_{F}A_{F}A_{L}G_{F}A_{L}G_{F}A_{L}A_{F}G_{F}C_{F}C_{F}U_{F}U_{F}A_{L}C_{F}U_{L}G_{F}G_{F}C_{L}G_{L}C_{F}
P-U_{F}U_{F}A_{F}A_{L}G_{F}A_{L}G_{F}A_{L}A_{F}G_{F}C_{F}C_{F}U_{F}UA_{L}C_{F}U_{L}G_{F}G_{F}C_{L}G_{L}C_{F}
P-U_{F}U_{F3}A_{F}A_{L}G_{F}A_{L}G_{F}A_{L}A_{F}G_{F}C_{F}C_{F}U_{F3}U_{F}A_{L}C_{F}U_{L3}G_{F}G_{F}C_{L}G_{L}C_{F}
P-U_{F}U_{F3}A_{F}A_{L}G_{F}A_{L}G_{F}A_{L}A_{F}G_{F}C_{F}C_{F4}U_{F3}U_{F}A_{L}C_{F}U_{L3}G_{F}G_{F}C_{L}G_{L}C_{F}
P-U_{F}U_{F3}A_{F}A_{L}G_{F}A_{L}G_{F}A_{L}A_{F}G_{F}C_{F}C_{F4}U_{F3}U_{F}A_{L}C_{F4}U_{L3}G_{F}G_{F}C_{L}G_{L}C_{F}
P-U_{F}U_{F3}A_{F}A_{L}G_{F}A_{L}G_{F}A_{L}A_{F}G_{F}C_{F4}C_{F4}U_{F3}U_{F}A_{L}C_{F4}U_{L3}G_{F}G_{F}C_{L}G_{L}C_{F}
P-U_{F}U_{F3}A_{F}A_{L2}G_{F}A_{L}G_{F}A_{L}A_{F}G_{F}C_{F4}C_{F4}U_{F3}U_{F}A_{L}C_{F4}U_{L3}G_{F}G_{F}C_{L}G_{L}C_{F}
P-U_{F}U_{F12}A_{F}A_{L}G_{F}A_{L}G_{F}A_{L}A_{F}G_{F}C_{F}C_{F}U_{F12}U_{F}A_{L}C_{F}U_{L12}G_{F}G_{F}C_{L}G_{L}C_{F}
P-U_{F}U_{F12}A_{F}A_{L}G_{F}A_{L}G_{F}A_{L}A_{F}G_{F}C_{F}C_{F4}U_{F12}U_{F}A_{L}C_{F}U_{L12}G_{F}G_{F}C_{L}G_{L}C_{F}
P-U_{F}U_{F12}A_{F}A_{L}G_{F}A_{L}G_{F}A_{L}A_{F}G_{F}C_{F}C_{F4}U_{F12}U_{F}A_{L}C_{F4}U_{L12}G_{F}G_{F}C_{L}G_{L}C_{F}
P-U_{F}U_{F12}A_{F}A_{L}G_{F}A_{L}G_{F}A_{L}A_{F}G_{F}C_{F4}C_{F4}U_{F12}U_{F}A_{L}C_{F4}U_{L12}G_{F}G_{F}C_{L}G_{L}C_{F}
P-U_{F}U_{F12}A_{F}A_{L2}G_{F}A_{L}G_{F}A_{L}A_{F}G_{F}C_{F4}C_{F4}U_{F12}U_{F}A_{L}C_{F4}U_{L12}G_{F}G_{F}C_{L}G_{L}C_{F}
P-U_{F}U_{F18}A_{F}A_{L}G_{F}A_{L}G_{F}A_{L}A_{FL G}_{F}C_{F}C_{F}U_{F18}U_{F}A_{L}C_{F}U_{L18}G_{F}G_{F}C_{L}G_{L}C_{F}
P-U_{F}U_{F18}A_{F}A_{L}G_{F}A_{L}G_{F}A_{L}A_{F}G_{F}C_{F}C_{F4}U_{F18}U_{F}A_{L}C_{F}U_{L18}G_{F}G_{F}C_{L}G_{L}C_{F}
P-U_{F}U_{F18}A_{F}A_{L}G_{F}A_{L}G_{F}A_{L}A_{F}G_{F}C_{F}C_{F4}U_{F18}U_{F}A_{L}C_{F4}U_{L18}G_{F}G_{F}C_{L}G_{L}C_{F}
P-U_{F}U_{F18}A_{F}A_{L}G_{F}A_{L}G_{F}A_{L}A_{F}G_{F}C_{F4}C_{F4}U_{F18}U_{F}A_{L}C_{F4}U_{L18}G_{F}G_{F}C_{L}G_{L}C_{F}
P-U_{F}U_{F18}A_{F}A_{L2}G_{F}A_{L}G_{F}A_{L}A_{F}G_{F}C_{F4}C_{F4}U_{F18}U_{F}A_{L}C_{F4}U_{L18}G_{F}G_{F}C_{L}G_{L}C_{F}
P-U_{F}U_{F3}A_{F}A_{L}G_{F}A_{L}G_{F}A_{L}A_{F}G_{F}C_{F}C_{F}U_{F3}UA_{L}C_{F}U_{L3}G_{F}G_{F}C_{L}G_{L}C_{F}
P-U_{F}U_{F3}A_{F}A_{L}G_{F}A_{L}G_{F}A_{L}A_{F}G_{F}C_{F}C_{F4}U_{F3}UA_{L}C_{F}U_{L3}G_{F}G_{F}C_{L}G_{L}C_{F}
P-U_{F}U_{F3}A_{F}A_{L}G_{F}A_{L}G_{F}A_{L}A_{F}G_{F}C_{F}C_{F4}U_{F3}UA_{L}C_{F4}U_{L3}G_{F}G_{F}C_{L}G_{L}C_{F}
P-U_{F}U_{F3}A_{F}A_{L}G_{F}A_{L}G_{F}A_{L}A_{F}G_{F}C_{F4}C_{F4}U_{F3}UA_{L}C_{F4}U_{L3}G_{F}G_{F}C_{L}G_{L}C_{F}
P-U_{F}U_{F3}A_{F}A_{L2}G_{F}A_{L}G_{F}A_{L}A_{F}G_{F}C_{F4}C_{F4}U_{F3}UA_{L}C_{F4}U_{L3}G_{F}G_{F}C_{L}G_{L}C_{F}
P-U_{F}U_{F12}A_{F}A_{L}G_{F}A_{L}G_{F}A_{L}A_{F}G_{F}C_{F}C_{F}U_{F12}UA_{L}C_{F}U_{L12}G_{F}G_{F}C_{L}G_{L}C_{F}
P-U_{F}U_{F12}A_{F}A_{L}G_{F}A_{L}G_{F}A_{L}A_{F}G_{F}C_{F}C_{F4}U_{F12}UA_{L}C_{F}U_{L12}G_{F}G_{F}C_{L}G_{L}C_{F}
P-U_{F}U_{F12}A_{F}A_{L}G_{F}A_{L}G_{F}A_{L}A_{F}G_{F}C_{F}C_{F4}U_{F12}UA_{L}C_{F4}U_{L12}G_{F}G_{F}C_{L}G_{L}C_{F}
P-U_{F}U_{F12}A_{F}A_{L}G_{F}A_{L}G_{F}A_{L}A_{F}G_{F}C_{F4}C_{F4}U_{F12}UA_{L}C_{F4}U_{L12}G_{F}G_{F}C_{L}G_{L}C_{F}
P-U_{F}U_{F12}A_{F}A_{L2}G_{F}A_{L}G_{F}A_{L}A_{F}G_{F}C_{F4}C_{F4}U_{F12}UA_{L}C_{F4}U_{L12}G_{F}G_{F}C_{L}G_{L}C_{F}
P-U_{F}U_{F18}A_{F}A_{L}G_{F}A_{L}G_{F}A_{L}A_{F}G_{F}C_{F}C_{F}U_{F18}UA_{L}C_{F}U_{L18}G_{F}G_{F}C_{L}G_{L}C_{F}
P-U_{F}U_{F18}A_{F}A_{L}G_{F}A_{L}G_{F}A_{L}A_{F}G_{F}C_{F}C_{F4}U_{F18}UA_{L}C_{F}U_{L18}G_{F}G_{F}C_{L}G_{L}C_{F}
P-U_{F}U_{F18}A_{F}A_{L}G_{F}A_{L}G_{F}A_{L}A_{F}G_{F}C_{F}C_{F4}U_{F18}UA_{L}C_{F4}U_{L18}G_{F}G_{F}C_{L}G_{L}C_{F}
P-U_{F}U_{F18}A_{F}A_{L}G_{F}A_{L}G_{F}A_{L}A_{F}G_{F}C_{F4}C_{F4}U_{F18}UA_{L}C_{F4}U_{L18}G_{F}G_{F}C_{L}G_{L}C_{F}
P-U_{F}U_{F18}A_{F}A_{L2}G_{F}A_{L}G_{F}A_{L}A_{F}G_{F}C_{F4}C_{F4}U_{F18}UA_{L}C_{F4}U_{L18}G_{F}G_{F}C_{L}G_{L}C_{F}
P-U_{F}U_{F}A_{L}A_{F}G_{L}A_{F}G_{L}A_{F}A_{L}G_{F}C_{F}C_{F}U_{L}U_{F}A_{F}C_{L}U_{F}G_{L}G_{F}C_{L}G_{L}C_{F}
P-U_{F}U_{F}A_{L}A_{F}G_{L}A_{F}G_{L}A_{F}A_{L}G_{F}C_{F}C_{F}U_{L}UA_{F}C_{L}U_{F}G_{L}G_{F}C_{L}G_{L}C_{F}
P-U_{F}U_{F3}A_{L}A_{F}G_{L}A_{F}G_{L}A_{F}A_{L}G_{F}C_{F}C_{F}U_{L3}U_{F}A_{F}C_{L}U_{F3}G_{L}G_{F}C_{L}G_{L}C_{F}
P-U_{F}U_{F3}A_{L}A_{F}G_{L}A_{F}G_{L}A_{F}A_{L}G_{F}C_{F}C_{F4}U_{L3}U_{F}A_{F}C_{L}U_{F3}G_{L}G_{F}C_{L}G_{L}C_{F}
P-U_{F}U_{F3}A_{L}A_{F}G_{L}A_{F}G_{L}A_{F}A_{L}G_{F}C_{F}C_{F4}U_{L3}U_{F}A_{F}C_{L4}U_{F3}G_{L}G_{F}C_{L}G_{L}C_{F}
P-U_{F}U_{F3}A_{L}A_{F}G_{L}A_{F}G_{L}A_{F}A_{L}G_{F}C_{F4}C_{F4}U_{L3}U_{F}A_{F}C_{L4}U_{F3}G_{L}G_{F}C_{L}G_{L}C_{F}
P-U_{F}U_{F3}A_{L}A_{F2}G_{L}A_{F}G_{L}A_{F}A_{L}G_{F}C_{F4}C_{F4}U_{L3}U_{F}A_{F}C_{L4}U_{F3}G_{L}G_{F}C_{L}G_{L}C_{F}
P-U_{F}U_{F12}A_{L}A_{F}G_{L}A_{F}G_{L}A_{F}A_{L}G_{F}C_{F}C_{F}U_{L12}U_{F}A_{F}C_{L}U_{F12}G_{L}G_{F}C_{L}G_{L}C_{F}
P-U_{F}U_{F12}A_{L}A_{F}G_{L}A_{F}G_{L}A_{F}A_{L}G_{F}C_{F}C_{F4}U_{L12}U_{F}A_{F}C_{L}U_{F12}G_{L}G_{F}C_{L}G_{L}C_{F}
P-U_{F}U_{F12}A_{L}A_{F}G_{L}A_{F}G_{L}A_{F}A_{L}G_{F}C_{F}C_{F4}U_{L12}U_{F}A_{F}C_{L4}U_{F12}G_{L}G_{F}C_{L}G_{L}C_{F}
P-U_{F}U_{F12}A_{L}A_{F}G_{L}A_{F}G_{L}A_{F}A_{L}G_{F}C_{F4}C_{F4}U_{L12}U_{F}A_{F}C_{L4}U_{F12}G_{L}G_{F}C_{L}G_{L}C_{F}
P-U_{F}U_{F12}A_{L}A_{F2}G_{L}A_{F}G_{L}A_{F}A_{L}G_{F}C_{F4}C_{F4}U_{L12}U_{F}A_{F}C_{L4}U_{F12}G_{L}G_{F}C_{L}G_{L}C_{F}
P-U_{F}U_{F18}A_{L}A_{F}G_{L}A_{F}G_{L}A_{F}A_{L}G_{F}C_{F}C_{F}U_{L18}U_{F}A_{F}C_{L}U_{F18}G_{L}G_{F}C_{L}G_{L}C_{F}
P-U_{F}U_{F18}A_{L}A_{F}G_{L}A_{F}G_{L}A_{F}A_{L}G_{F}C_{F}C_{F4}U_{L18}U_{F}A_{F}C_{L}U_{F18}G_{L}G_{F}C_{L}G_{L}C_{F}
P-U_{F}U_{F18}A_{L}A_{F}G_{L}A_{F}G_{L}A_{F}A_{L}G_{F}C_{F}C_{F4}U_{L18}U_{F}A_{F}C_{L4}U_{F18}G_{L}G_{F}C_{L}G_{L}C_{F}
P-U_{F}U_{F18}A_{L}A_{F}G_{L}A_{F}G_{L}A_{F}A_{L}G_{F}C_{F4}C_{F4}U_{L18}U_{F}A_{F}C_{L4}U_{F18}G_{L}G_{F}C_{L}G_{L}C_{F}
P-U_{F}U_{F18}A_{L}A_{F2}G_{L}A_{F}G_{L}A_{F}A_{L}G_{F}C_{F4}C_{F4}U_{L18}U_{F}A_{F}C_{L4}U_{F18}G_{L}G_{F}C_{L}G_{L}C_{F}
P-U_{F}U_{F3}A_{L}A_{F}G_{L}A_{F}G_{L}A_{F}A_{L}G_{F}C_{F}C_{F}U_{L3}UA_{F}C_{L}U_{F3}G_{L}G_{F}C_{L}G_{L}C_{F}
P-U_{F}U_{F3}A_{L}A_{F}G_{L}A_{F}G_{L}A_{F}A_{L}G_{F}C_{F}C_{F4}U_{L3}UA_{F}C_{L}U_{F3}G_{L}G_{F}C_{L}G_{L}C_{F}
P-U_{F}U_{F3}A_{L}A_{F}G_{L}A_{F}G_{L}A_{F}A_{L}G_{F}C_{F}C_{F4}U_{L3}UA_{F}C_{L4}U_{F3}G_{L}G_{F}C_{L}G_{L}C_{F}
P-U_{F}U_{F3}A_{L}A_{F}G_{L}A_{F}G_{L}A_{F}A_{L}G_{F}C_{F4}C_{F4}U_{L3}UA_{F}C_{L4}U_{F3}G_{L}G_{F}C_{L}G_{L}C_{F}
P-U_{F}U_{F3}A_{L}A_{F2}G_{L}A_{F}G_{L}A_{F}A_{L}G_{F}C_{F4}C_{F4}U_{L3}UA_{F}C_{L4}U_{F3}G_{L}G_{F}C_{L}G_{L}C_{F}
P-U_{F}U_{F12}A_{L}A_{F}G_{L}A_{F}G_{L}A_{F}A_{L}G_{F}C_{F}C_{F}U_{L12}UA_{F}C_{L}U_{F12}G_{L}G_{F}C_{L}G_{L}C_{F}
P-U_{F}U_{F12}A_{L}A_{F}G_{L}A_{F}G_{L}A_{F}A_{L}G_{F}C_{F}C_{F4}U_{L12}UA_{F}C_{L}U_{F12}G_{L}G_{F}C_{L}G_{L}C_{F}
P-U_{F}U_{F12}A_{L}A_{F}G_{L}A_{F}G_{L}A_{F}A_{L}G_{F}C_{F}C_{F4}U_{L12}UA_{F}C_{L4}U_{F12}G_{L}G_{F}C_{L}G_{L}C_{F}
P-U_{F}U_{F12}A_{L}A_{F}G_{L}A_{F}G_{L}A_{F}A_{L}G_{F}C_{F4}C_{F4}U_{L12}UA_{F}C_{L4}U_{F12}G_{L}G_{F}C_{L}G_{L}C_{F}
P-U_{F}U_{F12}A_{L}A_{F2}G_{L}A_{F}G_{L}A_{F}A_{L}G_{F}C_{F4}C_{F4}U_{L12}UA_{F}C_{L4}U_{F12}G_{L}G_{F}C_{L}G_{L}C_{F}
P-U_{F}U_{F18}A_{L}A_{F}G_{L}A_{F}G_{L}A_{F}A_{L}G_{F}C_{F}C_{F}U_{L18}UA_{F}C_{L}U_{F18}G_{L}G_{F}C_{L}G_{L}C_{F}
P-U_{F}U_{F18}A_{L}A_{F}G_{L}A_{F}G_{L}A_{F}A_{L}G_{F}C_{F}C_{F4}U_{L18}UA_{F}C_{L}U_{F18}G_{L}G_{F}C_{L}G_{L}C_{F}
P-U_{F}U_{F18}A_{L}A_{F}G_{L}A_{F}G_{L}A_{F}A_{L}G_{F}C_{F}C_{F4}U_{L18}UA_{F}C_{L4}U_{F18}G_{L}G_{F}C_{L}G_{L}C_{F}
P-U_{F}U_{F18}A_{L}A_{F}G_{L}A_{F}G_{L}A_{F}A_{L}G_{F}C_{F4}C_{F4}U_{L18}UA_{F}C_{L4}U_{F18}G_{L}G_{F}C_{L}G_{L}C_{F}
P-U_{F}U_{F18}A_{L}A_{F2}G_{L}A_{F}G_{L}A_{F}A_{L}G_{F}C_{F4}C_{F4}U_{L18}UA_{F}C_{L4}U_{F18}G_{L}G_{F}C_{L}G_{L}C_{F}
P-U_{F}U_{F}A_{F}A_{L}G_{F}A_{L}G_{F}A_{L}A_{F}G_{F}C_{F}C_{F}U_{L}U_{F}A_{L}C_{F}U_{F}G_{L}G_{F}C_{L}G_{L}C_{F}
P-U_{F}U_{F}A_{F}A_{L}G_{F}A_{L}G_{F}A_{L}A_{F}G_{F}C_{F}C_{F}U_{L}UA_{L}C_{F}U_{F}G_{L}G_{F}C_{L}G_{L}C_{F}
P-U_{F}U_{F3}A_{F}A_{L}G_{F}A_{L}G_{F}A_{L}A_{F}G_{F}C_{F}C_{F}U_{L3}U_{F}A_{L}C_{F}U_{F3}G_{L}G_{F}C_{L}G_{L}C_{F}
P-U_{F}U_{F3}A_{F}A_{L}G_{F}A_{L}G_{F}A_{L}A_{F}G_{F}C_{F}C_{F4}U_{L3}U_{F}A_{L}C_{F}U_{F3}G_{L}G_{F}C_{L}G_{L}C_{F}
P-U_{F}U_{F3}A_{F}A_{L}G_{F}A_{L}G_{F}A_{L}A_{F}G_{F}C_{F}C_{F4}U_{L3}U_{F}A_{L}C_{F4}U_{F3}G_{L}G_{F}C_{L}G_{L}C_{F}
P-U_{F}U_{F3}A_{F}A_{L}G_{F}A_{L}G_{F}A_{L}A_{F}G_{F}C_{F4}C_{F4}U_{L3}U_{F}A_{L}C_{F4}U_{F3}G_{L}G_{F}C_{L}G_{L}C_{F}
P-U_{F}U_{F3}A_{F}A_{L2}G_{F}A_{L}G_{F}A_{L}A_{F}G_{F}C_{F4}C_{F4}U_{L3}U_{F}A_{L}C_{F4}U_{F3}G_{L}G_{F}C_{L}G_{L}C_{F}
P-U_{F}U_{F12}A_{F}A_{L}G_{F}A_{L}G_{F}A_{L}A_{F}G_{F}C_{F}C_{F}U_{L12}U_{F}A_{L}C_{F}U_{F12}G_{L}G_{F}C_{L}G_{L}C_{F}
P-U_{F}U_{F12}A_{F}A_{L}G_{F}A_{L}G_{F}A_{L}A_{F}G_{F}C_{F}C_{F4}U_{L12}U_{F}A_{L}C_{F}U_{F12}G_{L}G_{F}C_{L}G_{L}C_{F}
P-U_{F}U_{F12}A_{F}A_{L}G_{F}A_{L}G_{F}A_{L}A_{F}G_{F}C_{F}C_{F4}U_{L12}U_{F}A_{L}C_{F4}U_{F12}G_{L}G_{F}C_{L}G_{L}C_{F}
P-U_{F}U_{F12}A_{F}A_{L}G_{F}A_{L}G_{F}A_{L}A_{F}G_{F}C_{F4}C_{F4}U_{L12}U_{F}A_{L}C_{F4}U_{F12}G_{L}G_{F}C_{L}G_{L}C_{F}
P-U_{F}U_{F12}A_{F}A_{L2}G_{F}A_{L}G_{F}A_{L}A_{F}G_{F}C_{F4}C_{F4}U_{L12}U_{F}A_{L}C_{F4}U_{F12}G_{L}G_{F}C_{L}G_{L}C_{F}
P-U_{F}U_{F18}A_{F}A_{L}G_{F}A_{L}G_{F}A_{L}A_{F}G_{F}C_{F}C_{F}U_{L18}U_{F}A_{L}C_{F}U_{F18}G_{L}G_{F}C_{L}G_{L}C_{F}
P-U_{F}U_{F18}A_{F}A_{L}G_{F}A_{L}G_{F}A_{L}A_{F}G_{F}C_{F}C_{F4}U_{L18}U_{F}A_{L}C_{F}U_{F18}G_{L}G_{F}C_{L}G_{L}C_{F}
P-U_{F}U_{F18}A_{F}A_{L}G_{F}A_{L}G_{F}A_{L}A_{F}G_{F}C_{F}C_{F4}U_{L18}U_{F}A_{L}C_{F4}U_{F18}G_{L}G_{F}C_{L}G_{L}C_{F}
P-U_{F}U_{F18}A_{F}A_{L}G_{F}A_{L}G_{F}A_{L}A_{F}G_{F}C_{F4}C_{F4}U_{L18}U_{F}A_{L}C_{F4}U_{F18}G_{L}G_{F}C_{L}G_{L}C_{F}
P-U_{F}U_{F18}A_{F}A_{L2}G_{F}A_{L}G_{F}A_{L}A_{F}G_{F}C_{F4}C_{F4}U_{L18}U_{F}A_{L}C_{F4}U_{F18}G_{L}G_{F}C_{L}G_{L}C_{F}
P-U_{F}U_{F3}A_{F}A_{L}G_{F}A_{L}G_{F}A_{L}A_{F}G_{F}C_{F}C_{F}U_{L3}UA_{L}C_{F}U_{F3}G_{L}G_{F}C_{L}G_{L}C_{F}
P-U_{F}U_{F3}A_{F}A_{L}G_{F}A_{L}G_{F}A_{L}A_{F}G_{F}C_{F}C_{F4}U_{L3}UA_{L}C_{F}U_{F3}G_{L}G_{F}C_{L}G_{L}C_{F}
P-U_{F}U_{F3}A_{F}A_{L}G_{F}A_{L}G_{F}A_{L}A_{F}G_{F}C_{F}C_{F4}U_{L3}UA_{L}C_{F4}U_{F3}G_{L}G_{F}C_{L}G_{L}C_{F}
P-U_{F}U_{F3}A_{F}A_{L}G_{F}A_{L}G_{F}A_{L}A_{F}G_{F}C_{F4}C_{F4}U_{L3}UA_{L}C_{F4}U_{F3}G_{L}G_{F}C_{L}G_{L}C_{F}
P-U_{F}U_{F3}A_{F}A_{L2}G_{F}A_{L}G_{F}A_{L}A_{F}G_{F}C_{F4}C_{F4}U_{L3}UA_{L}C_{F4}U_{F3}G_{L}G_{F}C_{L}G_{L}C_{F}
P-U_{F}U_{F12}A_{F}A_{L}G_{F}A_{L}G_{F}A_{L}A_{F}G_{F}C_{F}C_{F}U_{L12}UA_{L}C_{F}U_{F12}G_{L}G_{F}C_{L}G_{L}C_{F}
P-U_{F}U_{F12}A_{F}A_{L}G_{F}A_{L}G_{F}A_{L}A_{F}G_{F}C_{F}C_{F4}U_{L12}UA_{L}C_{F}U_{F12}G_{L}G_{F}C_{L}G_{L}C_{F}
P-U_{F}U_{F12}A_{F}A_{L}G_{F}A_{L}G_{F}A_{L}A_{F}G_{F}C_{F}C_{F4}U_{L12}UA_{L}C_{F4}U_{F12}G_{L}G_{F}C_{L}G_{L}C_{F}
P-U_{F}U_{F12}A_{F}A_{L}G_{F}A_{L}G_{F}A_{L}A_{F}G_{F}C_{F4}C_{F4}U_{L12}UA_{L}C_{F4}U_{F12}G_{L}G_{F}C_{L}G_{L}C_{F}
P-U_{F}U_{F12}A_{F}A_{L2}G_{F}A_{L}G_{F}A_{L}A_{F}G_{F}C_{F4}C_{F4}U_{L12}UA_{L}C_{F4}U_{F12}G_{L}G_{F}C_{L}G_{L}C_{F}
P-U_{F}U_{F18}A_{F}A_{L}G_{F}A_{L}G_{F}A_{L}A_{F}G_{F}C_{F}C_{F}U_{L18}UA_{L}C_{F}U_{F18}G_{L}G_{F}C_{L}G_{L}C_{F}
P-U_{F}U_{F18}A_{F}A_{L}G_{F}A_{L}G_{F}A_{L}A_{F}G_{F}C_{F}C_{F4}U_{L18}UA_{L}C_{F}U_{F18}G_{L}G_{F}C_{L}G_{L}C_{F}
P-U_{F}U_{F18}A_{F}A_{L}G_{F}A_{L}G_{F}A_{L}A_{F}G_{F}C_{F}C_{F4}U_{L18}UA_{L}C_{F4}U_{F18}G_{L}G_{F}C_{L}G_{L}C_{F}
P-U_{F}U_{F18}A_{F}A_{L}G_{F}A_{L}G_{F}A_{L}A_{F}G_{F}C_{F4}C_{F4}U_{L18}UA_{L}C_{F4}U_{F18}G_{L}G_{F}C_{L}G_{L}C_{F}
P-U_{F}U_{F18}A_{F}A_{L2}G_{F}A_{L}G_{F}A_{L}A_{F}G_{F}C_{F4}C_{F4}U_{L18}UA_{L}C_{F4}U_{F18}G_{L}G_{F}C_{L}G_{L}C_{F}

TABLE 16B
Compositions of Matter Suitable for Administration to a Subject
with or without a Protective Carrier and for Any Other use as
Provided for Herein (5′-3′)

P-U_{M}~U_{F3}~A_{M}A_{F}~G_{M}A_{F}~G_{M}A_{F}~A_{M}G_{F}~C_{M}C_{F}~U_{M3}U_{F}~A_{M}C_{F}~U_{M3}~G_{F}~G_{M}~A_{L}~A_{L}~G_{F}
P-U_{M}~U_{F3}~A_{M}A_{F}~G_{M}A_{F}~G_{M}A_{F}~A_{M}G_{F}~C_{M}C_{F4}~U_{M3}U_{F}~A_{M}C_{F}~U_{M3}~G_{F}~G_{M}~A_{L}~A_{L}~G_{F}
P-U_{M}~U_{F3}~A_{M}A_{F}~G_{M}A_{F}~G_{M}A_{F}~A_{M}G_{F}~C_{M}C_{F4}~U_{M3}U_{F}~A_{M}C_{F4}~U_{M3}~G_{F}~G_{M}~A_{L}~A_{L}~G_{F}
P-U_{M}~U_{F3}~A_{M}A_{F}~G_{M}A_{F}~G_{M}A_{F}~A_{M}G_{F}~C_{M4}C_{F4}~U_{M3}U_{F}~A_{M}C_{F4}~U_{M3}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M}C_{F}~U_{M12}U_{F}~A_{M}C_{F}~U_{M12}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M}C_{F4}~U_{M12}U_{F}~A_{M}C_{F}~U_{M12}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M}C_{F4}~U_{M12}U_{F}~A_{M}C_{F4}~U_{M12}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M4}C_{F4}~U_{M12}U_{F}~A_{M}C_{F4}~U_{M12}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M}C_{F}~U_{M18}U_{F}~A_{M}C_{F}~U_{M18}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M}C_{F4}~U_{M18}U_{F}~A_{M}C_{F}~U_{M18}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M}C_{F4}~U_{M18}U_{F}~A_{M}C_{F4}~U_{M18}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M4}C_{F4}~U_{M18}U_{F}~A_{M}C_{F4}~U_{M18}~G_{F}~G_{M}~A_{L}~A_{L}~G_{F}
P-U_{M}~U_{F3}~A_{M}A_{F}~G_{M}A_{F}~G_{M}A_{F}~A_{M}G_{F}~C_{M}C_{F}~U_{M3}U~A_{M}C_{F}~U_{M3}~G_{F}~G_{M}~A_{L}~A_{L}~G_{F}
P-U_{M}~U_{F3}~A_{M}A_{F}~G_{M}A_{F}~G_{M}A_{F}~A_{M}G_{F}~C_{M}C_{F4}~U_{M3}U~A_{M}C_{F}~U_{M3}~G_{F}~G_{M}~A_{L}~A_{L}~G_{F}
P-U_{M}~U_{F3}~A_{M}A_{F}~G_{M}A_{F}~G_{M}A_{F}~A_{M}G_{F}~C_{M}C_{F4}~U_{M3}U~A_{M}C_{F4}~U_{M3}~G_{F}~G_{M}~A_{L}~A_{L}~G_{F}
P-U_{M}~U_{F3}~A_{M}A_{F}~G_{M}A_{F}~G_{M}A_{F}~A_{M}G_{F}~C_{M4}C_{F4}~U_{M3}U~A_{M}C_{F4}~U_{M3}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M}C_{F}~U_{M12}U~A_{M}C_{F}~U_{M12}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M}C_{F4}~U_{M12}U~A_{M}C_{F}~U_{M12}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M}C_{F4}~U_{M12}U~A_{M}C_{F4}~U_{M12}~G_{F}~G_{M}~A_{L}~A_{L}~G_{F}
P-U_{M}~U_{F12}~A_{M}A_{F}~G_{M}A_{F}~G_{M}A_{F}~A_{M}G_{F}~C_{M4}C_{F4}~U_{M12}U~A_{M}C_{F4}~U_{M12}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M}C_{F}~U_{M18}U~A_{M}C_{F}~U_{M18}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M}C_{F4}~U_{M18}U~A_{M}C_{F}~U_{M18}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M}C_{F4}~U_{M18}U~A_{M}C_{F4}~U_{M18}~G_{F}~G_{M}~A_{L}~A_{L}~G_{F}
P-U_{M}~U_{F18}~A_{M}A_{F}~G_{M}A_{F}~G_{M}A_{F}~A_{M}G_{F}~C_{M4}C_{F4}~U_{M18}U~A_{M}C_{F4}~U_{M18}~G_{F}~G_{M}~A_{L}~A_{L}~G_{F}
P-U_{M}~U_{F}~A_{L}A_{F}~G_{M}A_{L}~G_{M}A_{F}~A_{L}G_{F}~C_{M}C_{F}~U_{M}U_{F}~A_{L}C_{F}~U_{M}~G_{L}~G_{M}~A_{L}~A_{L}~G_{F}
P-U_{M}~U_{F}~A_{L}A_{F}~G_{M}A_{L}~G_{M}A_{F}~A_{L}G_{F}~C_{M}C_{F}~U_{M}U~A_{L}C_{F}~U_{M}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M}C_{F}~U_{M3}U_{F}~A_{L}C_{F}~U_{M3}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M}C_{F4}~U_{M3}U_{F}~A_{L}C_{F}~U_{M3}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M}C_{F4}~U_{M3}U_{F}~A_{L}C_{F4}~U_{M3}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M4}C_{F4}~U_{M3}U_{F}~A_{L}C_{F4}~U_{M3}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M}C_{F}~U_{M12}U_{F}~A_{L}C_{F}~U_{M12}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M}C_{F4}~U_{M12}U_{F}~A_{L}C_{F}~U_{M12}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M}C_{F4}~U_{M12}U_{F}~A_{L}C_{F4}~U_{M12}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M4}C_{F4}~U_{M12}U_{F}~A_{L}C_{F4}~U_{M12}~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M}C_{F}~U_{M18}U_{F}~A_{L}C_{F}~U_{M}18~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M}C_{F4}~U_{M18}U_{F}~A_{L}C_{F}~U_{M18}~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M}C_{F4}~U_{M18}U_{F}~A_{L}C_{F4}~U_{M18}~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M4}C_{F4}~U_{M18}U_{F}~A_{L}C_{F4}~U_{M18}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M}C_{F}~U_{M3}U~A_{L}C_{F}~U_{M3}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M}C_{F4}~U_{M3}U~A_{L}C_{F}~U_{M3}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M}C_{F4}~U_{M3}U~A_{L}C_{F4}~U_{M3}~G_{L}~G_{M}~A_{L}~A_{L}~G_{F}
P-U_{M}~U_{F3}~A_{L}A_{F}~G_{M}A_{L}~G_{M}A_{F}~A_{L}G_{F}~C_{M4}C_{F4}~U_{M3}U~A_{L}C_{F4}~U_{M3}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M}C_{F}~U_{M12}U~A_{L}C_{F}~U_{M12}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M}C_{F4}~U_{M12}U~A_{L}C_{F}~U_{M12}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M}C_{F4}~U_{M12}U~A_{L}C_{F4}~U_{M12}~G_{L}~G_{M}~A_{L}~A_{L}~G_{F}
P-U_{M}~U_{F12}~A_{L}A_{F}~G_{M}A_{L}~G_{M}A_{F}~A_{L}G_{F}~C_{M4}C_{F4}~U_{M12}U~A_{L}C_{F4}~U_{M12}~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M}C_{F}~U_{M18}U~A_{L}C_{F}~U_{M18}~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M}C_{F4}~U_{M18}U~A_{L}C_{F}~U_{M18}~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M}C_{F4}~U_{M18}U~A_{L}C_{F4}~U_{M18}~G_{L}~G_{M}~A_{L}~A_{L}~G_{F}
P-U_{M}~U_{F18}~A_{L}A_{F}~G_{M}A_{L}~G_{M}A_{F}~A_{L}G_{F}~C_{M4}C_{F4}~U_{M18}U~A_{L}C_{F4}~U_{M18}~G_{L}~G_{M}~A_{L}~A_{L}~G_{F}
P-U_{M}~U_{F}~A_{M}A_{L}~G_{M}A_{L}~G_{M}A_{L}~A_{M}G_{F}~C_{M}C_{F}~U_{M}U_{F}~A_{L}C_{F}~U_{M}~G_{L}~G_{M}~A_{L}~A_{L}~G_{F}
P-U_{M}~U_{F}~A_{M}A_{L}~G_{M}A_{L}~G_{M}A_{L}~A_{M}G_{F}~C_{M}C_{F}~U_{M}U~A_{L}C_{F}~U_{M}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F}~U_{M3}U_{F}~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{M3}U_{F}~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{M3}U_{F}~A_{L}C_{F4}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M4}C_{F4}~U_{M3}U_{F}~A_{L}C_{F4}~U_{M3}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F}~U_{M12}U_{F}~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{M12}U_{F}~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{M12}U_{F}~A_{L}C_{F4}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M4}C_{F4}~U_{M12}U_{F}~A_{L}C_{F4}~U_{M12}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F}~U_{M18}U_{F}~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{M18}U_{F}~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{M18}U_{F}~A_{L}C_{F4}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M4}C_{F4}~U_{M18}U_{F}~A_{L}C_{F4}~U_{M18}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F}~U_{M3}U~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{M3}U~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{M3}U~A_{L}C_{F4}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M4}C_{F4}~U_{M3}U~A_{L}C_{F4}~U_{M3}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F}~U_{M12}U~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{M12}U~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{M12}U~A_{L}C_{F4}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M4}C_{F4}~U_{M12}U~A_{L}C_{F4}~U_{M12}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F}~U_{M18}U~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{M18}U~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{M18}U~A_{L}C_{F4}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M4}C_{F4}~U_{M18}U~A_{L}C_{F4}~U_{M18}~G_{L}~G_{M}~A_{L}~A_{L}~G_{F}
P-U_{M}~U_{F}~A_{L}A_{F}~G_{L}A_{F}~G_{L}A_{F}~A_{L}G_{F}~C_{M}C_{F}~U_{L}U_{F}~A_{M}C_{L}~U_{M}~G_{L}~G_{M}~A_{L}~A_{L}~G_{F}
P-U_{M}~U_{F}~A_{L}A_{F}~G_{L}A_{F}~G_{L}A_{F}~A_{L}G_{F}~C_{M}C_{F}~U_{L}U~A_{M}C_{L}~U_{M}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M}C_{F}~U_{L3}U_{F}~A_{M}C_{L}~U_{M3}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M}C_{F4}~U_{L3}U_{F}~A_{M}C_{L}~U_{M3}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M}C_{F4}~U_{L3}U_{F}~A_{M}C_{L4}~U_{M3}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M4}C_{F4}~U_{L3}U_{F}~A_{M}C_{L4}~U_{M3}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M}C_{F}~U_{L12}U_{F}~A_{M}C_{L}~U_{M12}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M}C_{F4}~U_{L12}U_{F}~A_{M}C_{L}~U_{M12}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M}C_{F4}~U_{L12}U_{F}~A_{M}C_{L4}~U_{M12}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M4}C_{F4}~U_{L12}U_{F}~A_{M}C_{L4}~U_{M12}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M}C_{F}~U_{L18}U_{F}~A_{M}C_{L}~U_{M18}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M}C_{F4}~U_{L18}U_{F}~A_{M}C_{L}~U_{M18}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M}C_{F4}~U_{L18}U_{F}~A_{M}C_{L4}~U_{M18}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M4}C_{F4}~U_{L18}U_{F}~A_{M}C_{L4}~U_{M18}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M}C_{F}~U_{L3}U~A_{M}C_{L}~U_{M3}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M}C_{F4}~U_{L3}U~A_{M}C_{L}~U_{M3}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M}C_{F4}~U_{L3}U~A_{M}C_{L4}~U_{M3}~G_{L}~G_{M}~A_{L}~A_{L}~G_{F}
P-U_{M}~U_{F3}~A_{L}A_{F}~G_{L}A_{F}~G_{L}A_{F}~A_{L}G_{F}~C_{M4}C_{F4}~U_{L3}U~A_{M}C_{L4}~U_{M3}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M}C_{F}~U_{L12}U~A_{M}C_{L}~U_{M12}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M}C_{F4}~U_{L12}U~A_{M}C_{L}~U_{M12}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M}C_{F4}~U_{L12}U~A_{M}C_{L4}~U_{M12}~G_{L}~G_{M}~A_{L}~A_{L}~G_{F}
P-U_{M}~U_{F12}~A_{L}A_{F}~G_{L}A_{F}~G_{L}A_{F}~A_{L}G_{F}~C_{M4}C_{F4}~U_{L12}U~A_{M}C_{L4}~U_{M12}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M}C_{F}~U_{L18}U~A_{M}C_{L}~U_{M18}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M}C_{F4}~U_{L18}U~A_{M}C_{L}~U_{M18}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M}C_{F4}~U_{L18}U~A_{M}C_{L4}~U_{M18}~G_{L}~G_{M}~A_{L}~A_{L}~G_{F}
P-U_{M}~U_{F18}~A_{L}A_{F}~G_{L}A_{F}~G_{L}A_{F}~A_{L}G_{F}~C_{M4}C_{F4}~U_{L18}U~A_{M}C_{L4}~U_{M18}~G_{L}~G_{M}~A_{L}~A_{L}~G_{F}
P-U_{M}~U_{F}~A_{M}A_{L}~G_{M}A_{L}~G_{M}A_{L}~A_{M}G_{F}~C_{M}C_{F}~U_{L}U_{F}~A_{L}C_{F}~U_{M}~G_{L}~G_{M}~A_{L}~A_{L}~G_{F}
P-U_{M}~U_{F}~A_{M}A_{L}~G_{M}A_{L}~G_{M}A_{L}~A_{M}G_{F}~C_{M}C_{F}~U_{L}U~A_{L}C_{F}~U_{M}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F}~U_{L3}U_{F}~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{L3}U_{F}~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{L3}U_{F}~A_{L}C_{F4}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M4}C_{F4}~U_{L3}U_{F}~A_{L}C_{F4}~U_{M3}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F}~U_{L12}U_{F}~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{L12}U_{F}~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{L12}U_{F}~A_{L}C_{F4}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M4}C_{F4}~U_{L12}U_{F}~A_{L}C_{F4}~U_{M12}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F}~U_{L18}U_{F}~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{L18}U_{F}~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{L18}U_{F}~A_{L}C_{F4}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M4}C_{F4}~U_{L18}U_{F}~A_{L}C_{F4}~U_{M18}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F}~U_{L3}U~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{L3}U~A_{L}C_{F}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M}C_{F4}~U_{L3}U~A_{L}C_{F4}~U_{M3}~G_{L}~G_{M}~A_{L}~A_{L}~G_{F}
P-U_{M}~U_{F3}~A_{M}A_{L}~G_{M}A_{L}~G_{M}A_{L}~A_{M}G_{F}~C_{M4}C_{F4}~U_{L3}U~A_{L}C_{F4}~U_{M3}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F}~U_{L12}U~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{L12}U~A_{L}C_{F}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M}C_{F4}~U_{L12}U~A_{L}C_{F4}~U_{M12}~G_{L}~G_{M}~A_{L}~A_{L}~G_{F}
P-U_{M}~U_{F12}~A_{M}A_{L}~G_{M}A_{L}~G_{M}A_{L}~A_{M}G_{F}~C_{M4}C_{F4}~U_{L12}U~A_{L}C_{F4}~U_{M12}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F}~U_{L18}U~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{L18}U~A_{L}C_{F}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M}C_{F4}~U_{L18}U~A_{L}C_{F4}~U_{M18}~G_{L}~G_{M}~A_{L}~A_{L}~G_{F}
P-U_{M}~U_{F18}~A_{M}A_{L}~G_{M}A_{L}~G_{M}A_{L}~A_{M}G_{F}~C_{M4}C_{F4}~U_{L18}U~A_{L}C_{F4}~U_{M18}~G_{L}~G_{M}~A_{L}~A_{L}~G_{F}

### Table 17

### Examples of Murine Apo-B ags-siRNA Compounds

AUUUCAGGAAUUGUUAAAG

Haringsma et al. (Nucleic Acids Res 40: 4125-36, 2012) used this sequence to generate ssRNAi compounds against murine APO-B target site 9470 including the following:

P-A_{F}U_{F}U_{F}U_{F}C_{F}A_{F}G_{F}G_{F}A_{F}A_{F}U_{F}U_{F}G_{F}U_{F}U_{F}A_{F}A_{F}A_{F}G_{F}U_{M}U_{M}

Any of the 5′-end nucleosides (P-A_{F}) shown in the examples of agsRNAi compounds in Table 17 can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 17 with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the nucleosides in Table 17 with an L modification found in the overhang precursor can have the L replaced with any of those units and linkages provided for herein for overhang precursors including one or more of the sugar or sugar analogs independently selected from the group consisting of: F, J, W, V, Y, T, TL and I.

Any of the nucleosides in Table 17 can have any of those units and linkages provided for herein for overhang precursors.

B. Examples of Application of AGSD to this Compound:

Compositions of Matter Suitable for Use in vitro
or in a Subject with a Protective Carrier (5′-3′)

P-A_{F}U_{F}U_{F3}U_{F3}C_{F}A_{F}G_{F}G_{F}A_{F}A_{F}U_{F}U_{F3}G_{F}U_{F}U_{F3}A_{F}FA_{F}G_{F}C_{L}G_{L}C_{F}
P-A_{F}U_{F3}U_{F3}U_{F}C_{F4}A_{F}G_{F}G_{F}A_{F}A_{F}U_{F}U_{F3}G_{F}U_{F}U_{F3}A_{F}A_{F}A_{F}G_{F}C_{L}G_{L}C_{F}
P-A_{F}U_{F3}U_{F3}U_{F3}C_{F}A_{F}G_{F}G_{F}A_{F}A_{F}U_{F}U_{F3}G_{F}U_{F}U_{F3}A_{F}A_{F}A_{F}G_{F}C_{L}G_{L}C_{F}
P-A_{F}U_{F3}U_{F3}U_{F3}C_{F}A_{F}G_{F}G_{F}A_{F}A_{F}U_{F3}U_{F3}G_{F}U_{F}U_{F3}A_{F}A_{F}A_{F}G_{F}C_{L}G_{L}C_{F}
P-A_{F}U_{F3}U_{F3}U_{F3}C_{F}A_{F}G_{F}G_{F}A_{F}A_{F}U_{F3}U_{F3}G_{F}U_{F3}U_{F3}A_{F}A_{F}A_{F}G_{F}C_{L}G_{L}C_{F}
P-A_{F}U_{F}U_{F12}U_{F12}C_{F}A_{F}G_{F}G_{F}A_{F}A_{F}U_{F}U_{F12}G_{F}U_{F}U_{F12}A_{F}A_{F}A_{F}G_{F}C_{L}G_{L}C_{F}
P-A_{F}U_{F12}U_{F12}U_{F}C_{F4}A_{F}G_{F}G_{F}A_{F}A_{F}U_{F}U_{F12}G_{F}U_{F}U_{F12}A_{F}A_{F}A_{F}G_{F}C_{L}G_{L}C_{F}
P-A_{F}U_{F12}U_{F12}U_{F12}C_{F}A_{F}G_{F}G_{F}A_{F}A_{F}U_{F}U_{F12}G_{F}U_{F}U_{F12}A_{F}A_{F}A_{F}G_{F}C_{L}G_{L}C_{F}
P-A_{F}U_{F12}U_{F12}U_{F12}C_{F}A_{F}G_{F}G_{F}A_{F}A_{F}U_{F12}U_{F12}G_{F}U_{F}U_{F12}A_{F}A_{F}A_{F}G_{F}C_{L}G_{L}C_{F}
P-A_{F}U_{F12}U_{F12}U_{F12}C_{F}A_{F}G_{F}G_{F}A_{F}A_{F}U_{F12}U_{F12}G_{F}U_{F12}U_{F12}A_{F}A_{F}A_{F}G_{F}C_{L}G_{L}C_{F}
P-A_{F}U_{F}U_{F18}U_{F18}C_{F}A_{F}G_{F}G_{F}A_{F}A_{F}U_{F}U_{F18}G_{F}U_{F}U_{F18}A_{F}A_{F}A_{F}G_{F}C_{L}G_{L}C_{F}
P-A_{F}U_{F18}U_{F18}U_{F}C_{F4}A_{F}G_{F}G_{F}A_{F}A_{F}U_{F}U_{F18}G_{F}U_{F}U_{F18}A_{F}A_{F}A_{F}G_{F}C_{L}G_{L}C_{F}
P-A_{F}U_{F18}U_{F18}U_{F18}C_{F}A_{F}G_{F}G_{F}A_{F}A_{F}U_{F}U_{F18}G_{F}U_{F}U_{F18}A_{F}A_{F}A_{F}G_{F}C_{L}G_{L}C_{F}
P-A_{F}U_{F18}U_{F18}U_{F18}C_{F}A_{F}G_{F}G_{F}A_{F}A_{F}U_{F18}U_{F18}G_{F}U_{F}U_{F18}A_{F}A_{F}A_{F}G_{F}C_{L}G_{L}C_{F}
P-A_{F}U_{F18}U_{F18}U_{F18}C_{F}A_{F}G_{F}G_{F}A_{F}A_{F}U_{F18}U_{F18}G_{F}U_{F18}U_{F18}A_{F}A_{F}A_{F}G_{F}C_{L}G_{L}C_{F}
P-A_{F}U_{F}U_{F3}U_{F3}C_{F}A_{F}G_{F}G_{F}A_{F}A_{F}U_{F}U_{F3}G_{F}UU_{F3}A_{F}A_{F}A_{F}G_{F}C_{L}G_{L}C_{F}
P-A_{F}U_{F3}U_{F3}U_{F}C_{F4}A_{F}G_{F}G_{F}A_{F}A_{F}U_{F}U_{F3}G_{F}UU_{F3}A_{F}A_{F}A_{F}G_{F}C_{L}G_{L}C_{F}
P-A_{F}U_{F3}U_{F3}U_{F3}C_{F}A_{F}G_{F}G_{F}A_{F}A_{F}U_{F}U_{F3}G_{F}UU_{F3}A_{F}A_{F}A_{F}G_{F}C_{L}G_{L}C_{F}
P-A_{F}U_{F3}U_{F3}U_{F3}C_{F}A_{F}G_{F}G_{F}A_{F}A_{F}U_{F3}U_{F3}G_{F}UU_{F3}A_{F}A_{F}A_{F}G_{F}C_{L}G_{L}C_{F}
P-A_{F}U_{F3}U_{F3}U_{F3}C_{F}A_{F}G_{F}G_{F}A_{F}A_{F}U_{F3}U_{F3}G_{F}U₃U_{F3}A_{F}A_{F}A_{F}G_{F}C_{L}G_{L}C_{F}
P-A_{F}U_{F}U_{F12}U_{F12}C_{F}A_{F}G_{F}G_{F}A_{F}A_{F}U_{F}U_{F12}G_{F}UU_{F12}A_{F}A_{F}A_{F}G_{F}C_{L}G_{L}C_{F}
P-A_{F}U_{F12}U_{F12}U_{F}C_{F4}A_{F}G_{F}G_{F}A_{F}A_{F}U_{F}U_{F12}G_{F}UU_{F12}A_{F}A_{F}A_{F}G_{F}C_{L}G_{L}C_{F}
P-A_{F}U_{F12}U_{F12}U_{F12}C_{F}A_{F}G_{F}G_{F}A_{F}A_{F}U_{F}U_{F12}G_{F}UU_{F12}A_{F}A_{F}A_{F}G_{F}C_{L}G_{L}C_{F}
P-A_{F}U_{F12}U_{F12}U_{F12}C_{F}A_{F}G_{F}G_{F}A_{F}A_{F}U_{F12}U_{F12}G_{F}UU_{F12}A_{F}A_{F}A_{F}G_{F}C_{L}G_{L}C_{F}
P-A_{F}U_{F12}U_{F12}U_{F12}C_{F}A_{F}G_{F}G_{F}A_{F}A_{F}U_{F12}U_{F12}G_{F}U₁₂U_{F12}A_{F}A_{F}A_{F}G_{F}C_{L}G_{L}C_{F}
P-A_{F}U_{F}U_{F18}U_{F18}C_{F}A_{F}G_{F}G_{F}A_{F}A_{F}U_{F}U_{F18}G_{F}UU_{F18}A_{F}A_{F}A_{F}G_{F}C_{L}G_{L}C_{F}
P-A_{F}U_{F18}U_{F18}U_{F}C_{F4}A_{F}G_{F}G_{F}A_{F}A_{F}U_{F}U_{F18}G_{F}UU_{F18}A_{F}A_{F}A_{F}G_{F}C_{L}G_{L}C_{F}
P-A_{F}U_{F18}U_{F18}U_{F18}C_{F}A_{F}G_{F}G_{F}A_{F}A_{F}U_{F}U_{F18}G_{F}UU_{F18}A_{F}A_{F}A_{F}G_{F}C_{L}G_{L}C_{F}
P-A_{F}U_{F18}U_{F18}U_{F18}C_{F}A_{F}G_{F}G_{F}A_{F}A_{F}U_{F18}U_{F18}G_{F}UU_{F18}A_{F}A_{F}A_{F}G_{F}C_{L}G_{L}C_{F}
P-A_{F}U_{F18}U_{F18}U_{F18}C_{F}A_{F}G_{F}G_{F}A_{F}A_{F}U_{F18}U_{F18}G_{F}U₁₈U_{F18}A_{F}A_{F}A_{F}G_{F}C_{L}G_{L}C_{F}
P-A_{F}U_{F}U_{F}U_{F}C_{F}A_{L}G_{F}G_{F}A_{L}A_{F}U_{F}U_{F}G_{L}U_{F}U_{F}A_{F}A_{L}A_{F}G_{F}C_{L}G_{L}C_{F}
P-A_{F}U_{F}U_{F}U_{F}C_{F}A_{L}G_{F}G_{F}A_{L}A_{F}U_{F}U_{F}G_{L}U U_{F}A_{F}A_{L}A_{F}G_{F}C_{L}G_{L}C_{F}
P-A_{F}U_{F}U_{F3}U_{F3}C_{F}A_{L}G_{F}G_{F}A_{L}A_{F}U_{F}U_{F3}G_{L}U_{F}U_{F3}A_{F}A_{L}A_{F}G_{F}C_{L}G_{L}C_{F}
P-A_{F}U_{F3}U_{F3}U_{F}C_{F4}A_{L}G_{F}G_{F}A_{L}A_{F}U_{F}U_{F3}G_{L}U_{F}U_{F3}A_{F}A_{L}A_{F}G_{F}C_{L}G_{L}C_{F}
P-A_{F}U_{F3}U_{F3}U_{F3}C_{F}A_{L}G_{F}G_{F}A_{L}A_{F}U_{F}U_{F3}G_{L}U_{F}U_{F3}A_{F}A_{L}A_{F}G_{F}C_{L}G_{L}C_{F}
P-A_{F}U_{F3}U_{F3}U_{F3}C_{F}A_{L}G_{F}G_{F}A_{L}A_{F}U_{F3}U_{F3}G_{L}U_{F}U_{F3}A_{F}A_{L}A_{F}G_{F}C_{L}G_{L}C_{F}
P-A_{F}U_{F3}U_{F3}U_{F3}C_{F}A_{L}G_{F}G_{F}A_{L}A_{F}U_{F3}U_{F3}G_{L}U_{F3}U_{F3}A_{F}A_{L}A_{F}G_{F}C_{L}G_{L}C_{F}
P-A_{F}U_{F}U_{F12}U_{F12}C_{F}A_{L}G_{F}G_{F}A_{L}A_{F}U_{F}U_{F12}G_{L}U_{F}U_{F12}A_{F}A_{L}A_{F}G_{F}C_{L}G_{L}C_{F}
P-A_{F}U_{F12}U_{F12}U_{F}C_{F4}A_{L}G_{F}G_{F}A_{L}A_{F}U_{F}U_{F12}G_{L}U_{F}U_{F12}A_{F}A_{L}A_{F}G_{F}C_{L}G_{L}C_{F}
P-A_{F}U_{F12}U_{F12}U_{F12}C_{F}A_{L}G_{F}G_{F}A_{L}A_{F}U_{F}U_{F12}G_{L}U_{F}U_{F12}A_{F}A_{L}A_{F}G_{F}C_{L}G_{L}C_{F}
P-A_{F}U_{F12}U_{F12}U_{F12}C_{F}A_{L}G_{F}G_{F}A_{L}A_{F}U_{F12}U_{F12}G_{L}U_{F}U_{F12}A_{F}A_{L}A_{F}G_{F}C_{L}G_{L}C_{F}
P-A_{F}U_{F12}U_{F12}U_{F12}C_{F}A_{L}G_{F}G_{F}A_{L}A_{F}U_{F12}U_{F12}G_{L}U_{F12}U_{F12}A_{F}A_{L}A_{F}G_{F}C_{L}G_{L}C_{F}
P-A_{F}U_{F}U_{F18}U_{F18}C_{F}A_{L}G_{F}G_{F}A_{L}A_{F}U_{F}U_{F18}G_{L}U_{F}U_{F18}A_{F}A_{L}A_{F}G_{F}C_{L}G_{L}C_{F}
P-A_{F}U_{F18}U_{F18}U_{F}C_{F4}A_{L}G_{F}G_{F}A_{L}A_{F}U_{F}U_{F18}G_{L}U_{F}U_{F18}A_{F}A_{L}A_{F}G_{F}C_{L}G_{L}C_{F}
P-A_{F}U_{F18}U_{F18}U_{F18}C_{F}A_{L}G_{F}G_{F}A_{L}A_{F}U_{F}U_{F18}G_{L}U_{F}U_{F18}A_{F}A_{L}A_{F}G_{F}C_{L}G_{L}C_{F}
P-A_{F}U_{F18}U_{F18}U_{F18}C_{F}A_{L}G_{F}G_{F}A_{L}A_{F}U_{F18}U_{F18}G_{L}U_{F}U_{F18}A_{F}A_{L}A_{F}G_{F}C_{L}G_{L}C_{F}
P-A_{F}U_{F18}U_{F18}U_{F18}C_{F}A_{L}G_{F}G_{F}A_{L}A_{F}U_{F18}U_{F18}G_{L}U_{F18}U_{F18}A_{F}A_{L}A_{F}G_{F}C_{L}G_{L}C_{F}
P-A_{F}U_{F}U_{F3}U_{F3}C_{F}A_{L}G_{F}G_{F}A_{L}A_{F}U_{F}U_{F3}G_{L}UU_{F3}A_{F}A_{L}A_{F}G_{F}C_{L}G_{L}C_{F}
P-A_{F}U_{F3}U_{F3}U_{F}C_{F4}A_{L}G_{F}G_{F}A_{L}A_{F}U_{F}U_{F3}G_{L}U U_{F3}A_{F}A_{L}A_{F}G_{F}C_{L}G_{L}C_{F}
P-A_{F}U_{F3}U_{F3}U_{F3}C_{F}A_{L}G_{F}G_{F}A_{L}A_{F}U_{F}U_{F3}G_{L}UU_{F3}A_{F}A_{L}A_{F}G_{F}C_{L}G_{L}C_{F}
P-A_{F}U_{F3}U_{F3}U_{F3}C_{F}A_{L}G_{F}G_{F}A_{L}A_{F}U_{F3}U_{F3}G_{L}UU_{F3}A_{F}A_{L}A_{F}G_{F}C_{L}G_{L}C_{F}
P-A_{F}U_{F3}U_{F3}U_{F3}C_{F}A_{L}G_{F}G_{F}A_{L}A_{F}U_{F3}U_{F3}G_{L}U₃U_{F3}A_{F}A_{L}A_{F}G_{F}C_{L}G_{L}C_{F}
P-A_{F}U_{F}U_{F12}U_{F12}C_{F}A_{L}G_{F}G_{F}A_{L}A_{F}U_{F}U_{F12}G_{L}UU_{F12}A_{F}A_{L}A_{F}G_{F}C_{L}G_{L}C_{F}
P-A_{F}U_{F12}U_{F12}U_{F}C_{F4}A_{L}G_{F}G_{F}A_{L}A_{F}U_{F}U_{F12}G_{L}UU_{F12}A_{F}A_{L}A_{F}G_{F}C_{L}G_{L}C_{F}
P-A_{F}U_{F12}U_{F12}U_{F12}C_{F}A_{L}G_{F}G_{F}A_{L}A_{F}U_{F}U_{F12}G_{L}UU_{F12}A_{F}A_{L}A_{F}G_{F}C_{L}G_{L}C_{F}
P-A_{F}U_{F12}U_{F12}U_{F12}C_{F}A_{L}G_{F}G_{F}A_{L}A_{F}U_{F12}U_{F12}G_{L}UU_{F12}A_{F}A_{L}A_{F}G_{F}C_{L}G_{L}C_{F}
P-A_{F}U_{F12}U_{F12}U_{F12}C_{F}A_{L}G_{F}G_{F}A_{L}A_{F}U_{F12}U_{F12}G_{L}U₁₂U_{F12}A_{F}A_{L}A_{F}G_{F}C_{L}G_{L}C_{F}
P-A_{F}U_{F}U_{F18}U_{F18}C_{F}A_{L}G_{F}G_{F}A_{L}A_{F}U_{F}U_{F18}G_{L}UU_{F18}A_{F}A_{L}A_{F}G_{F}C_{L}G_{L}C_{F}
P-A_{F}U_{F18}U_{F18}U_{F}C_{F4}A_{L}G_{F}G_{F}A_{L}A_{F}U_{F}U_{F18}G_{L}UU_{F18}A_{F}A_{L}A_{F}G_{F}C_{L}G_{L}C_{F}
P-A_{F}U_{F18}U_{F18}U_{F18}C_{F}A_{L}G_{F}G_{F}A_{L}A_{F}U_{F}U_{F18}G_{L}UU_{F18}A_{F}A_{L}A_{F}G_{F}C_{L}G_{L}C_{F}
P-A_{F}U_{F18}U_{F18}U_{F18}C_{F}A_{L}G_{F}G_{F}A_{L}A_{F}U_{F18}U_{F18}G_{L}UU_{F18}A_{F}A_{L}A_{F}G_{F}C_{L}G_{L}C_{F}
P-A_{F}U_{F18}U_{F18}U_{F18}C_{F}A_{L}G_{F}G_{F}A_{L}A_{F}U_{F18}U_{F18}G_{L}U₁₈U_{F18}A_{F}A_{L}A_{F}G_{F}C_{L}G_{L}C_{F}
P-A_{F}U_{F}U_{F}U_{F}C_{L}A_{F}G_{F}G_{L}A_{F}A_{F}U_{F}U_{L}G_{F}U_{F}U_{L}A_{F}A_{F}A_{L}G_{F}C_{L}G_{L}C_{F}
P-A_{F}U_{F}U_{F}U_{F}C_{L}A_{F}G_{F}G_{L}A_{F}A_{F}U_{F}U_{L}G_{F}UU_{L}A_{F}A_{F}A_{L}G_{F}C_{L}G_{L}C_{F}
P-A_{F}U_{F}U_{F3}U_{F3}CLA_{F}G_{F}G_{L}A_{F}A_{F}U_{F}U_{L3}G_{F}U_{F}U_{L3}A_{F}A_{F}A_{L}G_{F}C_{L}G_{L}C_{F}
P-A_{F}U_{F3}U_{F3}U_{F}C_{L4}A_{F}G_{F}G_{L}A_{F}A_{F}U_{F}U_{L3}G_{F}U_{F}U_{L3}A_{F}A_{F}A_{L}G_{F}C_{L}G_{L}C_{F}
P-A_{F}U_{F3}U_{F3}U_{F3}C_{L}A_{F}G_{F}G_{L}A_{F}A_{F}U_{F}U_{L3}G_{F}U_{F}U_{L3}A_{F}A_{F}A_{L}G_{F}C_{L}G_{L}C_{F}
P-A_{F}U_{F3}U_{F3}U_{F3}C_{L}A_{F}G_{F}G_{L}A_{F}A_{F}U_{F3}U_{L3}G_{F}U_{F}U_{L3}A_{F}A_{F}A_{L}G_{F}C_{L}G_{L}C_{F}
P-A_{F}U_{F3}U_{F3}U_{F3}C_{L}A_{F}G_{F}G_{L}A_{F}A_{F}U_{F3}U_{L3}G_{F}U_{F3}U_{L3}A_{F}A_{F}A_{L}G_{F}C_{L}G_{L}C_{F}
P-A_{F}U_{F}U_{F12}U_{F12}C_{L}A_{F}G_{F}G_{L}A_{F}A_{F}U_{F}U_{L12}G_{F}U_{F}U_{L12}A_{F}A_{F}A_{L}G_{F}C_{L}G_{L}C_{F}
P-A_{F}U_{F12}U_{F12}U_{F}C_{L4}AFG_{F}G_{L}A_{F}A_{F}U_{F}U_{L12}G_{F}U_{F}U_{L12}A_{F}A_{F}A_{L}G_{F}C_{L}G_{L}C_{F}
P-A_{F}U_{F12}U_{F12}U_{F12}C_{L}A_{F}G_{F}G_{L}A_{F}A_{F}U_{F}U_{L12}G_{F}U_{F}U_{L12}A_{F}A_{F}A_{L}G_{F}C_{L}G_{L}C_{F}
P-A_{F}U_{F12}U_{F12}U_{F12}C_{L}A_{F}G_{F}G_{L}A_{F}A_{F}U_{F12}U_{L12}G_{F}U_{F}U_{L12}A_{F}A_{F}A_{L}G_{F}C_{L}G_{L}C_{F}
P-A_{F}U_{F12}U_{F12}U_{F12}C_{L}A_{F}G_{F}G_{L}A_{F}A_{F}U_{F12}U_{L12}G_{F}U_{F12}U_{L12}A_{F}A_{F}A_{L}G_{F}C_{L}G_{L}C_{F}
P-A_{F}U_{F}U_{F18}U_{F18}C_{L}A_{F}G_{F}G_{L}A_{F}A_{F}U_{F}U_{L18}G_{F}U_{F}U_{L18}A_{F}A_{F}A_{L}G_{F}C_{L}G_{L}C_{F}
P-A_{F}U_{F18}U_{F18}U_{F}C_{L4}A_{F}G_{F}G_{L}A_{F}A_{F}U_{F}U_{L18}G_{F}U_{F}U_{L18}A_{F}A_{F}A_{L}G_{F}C_{L}G_{L}C_{F}
P-A_{F}U_{F18}U_{F18}U_{F18}C_{L}A_{F}G_{F}G_{L}A_{F}A_{F}U_{F}U_{L18}G_{F}U_{F}U_{L18}A_{F}A_{F}A_{L}G_{F}C_{L}G_{L}C_{F}
P-A_{F}U_{F18}U_{F18}U_{F18}C_{L}A_{F}G_{F}G_{L}A_{F}A_{F}U_{F18}U_{L18}G_{F}U_{F}U_{L18}A_{F}A_{F}A_{L}G_{F}C_{L}G_{L}C_{F}
P-A_{F}U_{F18}U_{F18}U_{F18}C_{L}A_{F}G_{F}G_{L}A_{F}A_{F}U_{F18}U_{L18}G_{F}U_{F18}U_{L18}A_{F}A_{F}A_{L}G_{F}C_{L}G_{L}C_{F}
P-A_{F}U_{F}U_{F3}U_{F3}C_{L}A_{F}G_{F}G_{L}A_{F}A_{F}U_{F}U_{L3}G_{F}UU_{L3}A_{F}A_{F}A_{L}G_{F}C_{L}G_{L}C_{F}
P-A_{F}U_{F3}U_{F3}U_{F}C_{L4}A_{F}G_{F}G_{L}A_{F}A_{F}U_{F}U_{L3}G_{F}UU_{L3}A_{F}A_{F}A_{L}G_{F}C_{L}G_{L}C_{F}
P-A_{F}U_{F3}U_{F3}U_{F3}C_{L}A_{F}G_{F}G_{L}A_{F}A_{F}U_{F}U_{L3}G_{F}UU_{L3}A_{F}A_{F}A_{L}G_{F}C_{L}G_{L}C_{F}
P-A_{F}U_{F3}U_{F3}U_{F3}C_{L}A_{F}G_{F}G_{L}A_{F}A_{F}U_{F3}U_{L3}G_{F}UU_{L3}A_{F}A_{F}A_{L}G_{F}C_{L}G_{L}C_{F}
P-A_{F}U_{F3}U_{F3}U_{F3}C_{L}A_{F}G_{F}G_{L}A_{F}A_{F}U_{F3}U_{L3}G_{F}U₃U_{L3}A_{F}A_{F}A_{L}G_{F}C_{L}G_{L}C_{F}
P-A_{F}U_{F}U_{F12}U_{F12}C_{L}A_{F}G_{F}G_{L}A_{F}A_{F}U_{F}U_{L12}G_{F}UU_{L12}A_{F}A_{F}A_{L}G_{F}C_{L}G_{L}C_{F}
P-A_{F}U_{F12}U_{F12}U_{F}C_{L4}A_{F}G_{F}G_{L}A_{F}A_{F}U_{F}U_{L12}G_{F}UU_{L12}A_{F}A_{F}A_{L}G_{F}C_{L}G_{L}C_{F}
P-A_{F}U_{F12}U_{F12}U_{F12}C_{L}A_{F}G_{F}G_{L}A_{F}A_{F}U_{F}U_{L12}G_{F}UU_{L12}A_{F}A_{F}A_{L}G_{F}C_{L}G_{L}C_{F}
P-A_{F}U_{F12}U_{F12}U_{F12}C_{L}A_{F}G_{F}G_{L}A_{F}A_{F}U_{F12}U_{L12}G_{F}UU_{L12}A_{F}A_{F}A_{L}G_{F}C_{L}G_{L}C_{F}
P-A_{F}U_{F12}U_{F12}U_{F12}C_{L}A_{F}G_{F}G_{L}A_{F}A_{F}U_{F12}U_{L12}G_{F}U₁₂U_{L12}A_{F}A_{F}A_{L}G_{F}C_{L}G_{L}C_{F}
P-A_{F}U_{F}U_{F18}U_{F18}C_{L}A_{F}G_{F}G_{L}A_{F}A_{F}U_{F}U_{L18}G_{F}UU_{L18}A_{F}A_{F}A_{L}G_{F}C_{L}G_{L}C_{F}
P-A_{F}U_{F18}U_{F18}U_{F}C_{L}4A_{F}G_{F}G_{L}A_{F}A_{F}U_{F}U_{L18}G_{F}UU_{L18}A_{F}A_{F}A_{L}G_{F}C_{L}G_{L}C_{F}
P-A_{F}U_{F18}U_{F18}U_{F18}C_{L}A_{F}G_{F}G_{L}A_{F}A_{F}U_{F}U_{L18}G_{F}UU_{L18}A_{F}A_{F}A_{L}G_{F}C_{L}G_{L}C_{F}
P-A_{F}U_{F18}U_{F18}U_{F18}C_{L}A_{F}G_{F}G_{L}A_{F}A_{F}U_{F18}U_{L18}G_{F}UU_{L18}A_{F}A_{F}A_{L}G_{F}C_{L}G_{L}C_{F}
P-A_{F}U_{F18}U_{F18}U_{F18}C_{L}A_{F}G_{F}G_{L}A_{F}A_{F}U_{F18}U_{L18}G_{F}U₁₈U_{L18}A_{F}A_{F}A_{L}G_{F}C_{L}G_{L}C_{F}
P-A_{F}U_{F}U_{L}U_{F}C_{F}A_{F}G_{L}G_{F}A_{L}A_{F}U_{F}U_{L}G_{F}U_{F}U_{F}A_{L}A_{F}A_{L}G_{F}C_{L}G_{L}C_{F}
P-A_{F}U_{F}U_{L}U_{F}C_{F}A_{F}G_{L}G_{F}A_{L}A_{F}U_{F}U_{L}G_{F}UU_{F}A_{L}A_{F}A_{L}G_{F}C_{L}G_{L}C_{F}
P-A_{F}U_{F}U_{L3}U_{F3}C_{F}A_{F}G_{L}G_{F}A_{L}A_{F}U_{F}U_{L3}G_{F}U_{F}U_{F3}A_{L}A_{F}A_{L}G_{F}C_{L}G_{L}C_{F}
P-A_{F}U_{F3}U_{L3}U_{F}C_{F4}A_{F}G_{L}G_{F}A_{L}A_{F}U_{F}U_{L3}G_{F}U_{F}U_{F3}A_{L}A_{F}A_{L}G_{F}C_{L}G_{L}C_{F}
P-A_{F}U_{F3}U_{L3}U_{F3}C_{F}A_{F}G_{L}G_{F}A_{L}A_{F}U_{F}U_{L3}G_{F}U_{F}U_{F3}A_{L}A_{F}A_{L}G_{F}C_{L}G_{L}C_{F}
P-A_{F}U_{F3}U_{L3}U_{F3}C_{F}A_{F}G_{L}G_{F}A_{L}A_{F}U_{F3}U_{L3}G_{F}U_{F}U_{F3}A_{L}A_{F}A_{L}G_{F}C_{L}G_{L}C_{F}
P-A_{F}U_{F3}U_{L3}U_{F3}C_{F}A_{F}G_{L}G_{F}A_{L}A_{F}U_{F3}U_{L3}G_{F}U_{F3}U_{F3}A_{L}A_{F}A_{L}G_{F}C_{L}G_{L}C_{F}
P-A_{F}U_{F}U_{L12}U_{F12}C_{F}A_{F}G_{L}G_{F}A_{L}A_{F}U_{F}U_{L12}G_{F}U_{F}U_{F12}A_{L}A_{F}A_{L}G_{F}C_{L}G_{L}C_{F}
P-A_{F}U_{F12}U_{L12}U_{F}C_{F4}A_{F}G_{L}G_{F}A_{L}A_{F}U_{F}U_{L12}G_{F}U_{F}U_{F12}A_{L}A_{F}A_{L}G_{F}C_{L}G_{L}C_{F}
P-A_{F}U_{F12}U_{L12}U_{F12}C_{F}A_{F}G_{L}G_{F}A_{L}A_{F}U_{F}U_{L12}G_{F}U_{F}U_{F12}A_{L}A_{F}A_{L}G_{F}C_{L}G_{L}C_{F}
P-A_{F}U_{F12}U_{L12}U_{F12}C_{F}A_{F}G_{L}G_{F}A_{L}A_{F}U_{F12}U_{L12}G_{F}U_{F}U_{F12}A_{L}A_{F}A_{L}G_{F}C_{L}G_{L}C_{F}
P-A_{F}U_{F12}U_{L12}U_{F12}C_{F}A_{F}G_{L}G_{F}A_{L}A_{F}U_{F12}U_{L12}G_{F}U_{F12}U_{F12}A_{L}A_{F}A_{L}G_{F}C_{L}G_{L}C_{F}
P-A_{F}U_{F}U_{L18}U_{F18}C_{F}A_{F}G_{L}G_{F}A_{L}A_{F}U_{F}U_{L18}G_{F}U_{F}U_{F18}A_{L}A_{F}A_{L}G_{F}C_{L}G_{L}C_{F}
P-A_{F}U_{F18}U_{L18}U_{F}C_{F4}A_{F}G_{L}G_{F}A_{L}A_{F}U_{F}U_{L18}G_{F}U_{F}U_{F18}A_{L}A_{F}A_{L}G_{F}C_{L}G_{L}C_{F}
P-A_{F}U_{F18}U_{L18}U_{F18}C_{F}A_{F}G_{L}G_{F}A_{L}A_{F}U_{F}U_{L18}G_{F}U_{F}U_{F18}A_{L}A_{F}A_{L}G_{F}C_{L}G_{L}C_{F}
P-A_{F}U_{F18}U_{L18}U_{F18}C_{F}A_{F}G_{L}G_{F}A_{L}A_{F}U_{F18}U_{L18}G_{F}U_{F}U_{F18}A_{L}A_{F}A_{L}G_{F}C_{L}G_{L}C_{F}
P-A_{F}U_{F18}U_{L18}U_{F18}C_{F}A_{F}G_{L}G_{F}A_{L}A_{F}U_{F18}U_{L18}G_{F}U_{F18}U_{F18}A_{L}A_{F}A_{L}G_{F}C_{L}G_{L}C_{F}
P-A_{F}U_{F}U_{L3}U_{F3}C_{F}A_{F}G_{L}G_{F}A_{L}A_{F}U_{F}U_{L3}G_{F}UU_{F3}A_{L}A_{F}A_{L}G_{F}C_{L}G_{L}C_{F}
P-A_{F}U_{F3}U_{L3}U_{F}C_{F4}A_{F}G_{L}G_{F}A_{L}A_{F}U_{F}U_{L3}G_{F}UU_{F3}A_{L}A_{F}A_{L}G_{F}C_{L}G_{L}C_{F}
P-A_{F}U_{F3}U_{L3}U_{F3}C_{F}A_{F}G_{L}G_{F}A_{L}A_{F}U_{F}U_{L3}G_{F}UU_{F3}A_{L}A_{F}A_{L}G_{F}C_{L}G_{L}C_{F}
P-A_{F}U_{F3}U_{L3}U_{F3}C_{F}A_{F}G_{L}G_{F}A_{L}A_{F}U_{F3}U_{L3}G_{F}UU_{F3}A_{L}A_{F}A_{L}G_{F}C_{L}G_{L}C_{F}
P-A_{F}U_{F3}U_{L3}U_{F3}C_{F}A_{F}G_{L}G_{F}A_{L}A_{F}U_{F3}U_{L3}G_{F}U₃U_{F3}A_{L}A_{F}A_{L}G_{F}C_{L}G_{L}C_{F}
P-A_{F}U_{F}U_{L12}U_{F12}C_{F}A_{F}G_{L}G_{F}A_{L}A_{F}U_{F}U_{L12}G_{F}UU_{F12}A_{L}A_{F}A_{L}G_{F}C_{L}G_{L}C_{F}
P-A_{F}U_{F12}U_{L12}U_{F}C_{F4}A_{F}G_{L}G_{F}A_{L}A_{F}U_{F}U_{L12}G_{F}UU_{F12}A_{L}A_{F}A_{L}G_{F}C_{L}G_{L}C_{F}
P-A_{F}U_{F12}U_{L12}U_{F12}C_{F}A_{F}G_{L}G_{F}A_{L}A_{F}U_{F}U_{L12}G_{F}UU_{F12}A_{L}A_{F}A_{L}G_{F}C_{L}G_{L}C_{F}
P-A_{F}U_{F12}U_{F12}U_{F12}C_{F}A_{F}G_{L}G_{F}A_{L}A_{F}U_{F12}U_{L12}G_{F}UU_{F12}A_{L}A_{F}A_{L}G_{F}C_{L}G_{L}C_{F}
P-A_{F}U_{F12}U_{L12}U_{F12}C_{F}A_{F}G_{L}G_{F}A_{L}A_{F}U_{F12}U_{L12}G_{F}U₁₂U_{F12}A_{L}A_{F}A_{L}G_{F}C_{L}G_{L}C_{F}
P-A_{F}U_{F}U_{L18}U_{F18}C_{F}A_{F}G_{L}G_{F}A_{L}A_{F}U_{F}U_{L18}G_{F}UU_{F18}A_{L}A_{F}A_{L}G_{F}C_{L}G_{L}C_{F}
P-A_{F}U_{F18}U_{L18}U_{F}C_{F4}A_{F}G_{L}G_{F}A_{L}A_{F}U_{F}U_{L18}G_{F}UU_{F18}A_{L}A_{F}A_{L}G_{F}C_{L}G_{L}C_{F}
P-A_{F}U_{F18}U_{L18}U_{F18}C_{F}A_{F}G_{L}G_{F}A_{L}A_{F}U_{F}U_{L18}G_{F}UU_{F18}A_{L}A_{F}A_{L}G_{F}C_{L}G_{L}C_{F}
P-A_{F}U_{F18}U_{L18}U_{F18}C_{F}A_{F}G_{L}G_{F}A_{L}A_{F}U_{F18}U_{L18}G_{F}UU_{F18}A_{L}A_{F}A_{L}G_{F}C_{L}G_{L}C_{F}
P-A_{F}U_{F18}U_{L18}U_{F18}C_{F}A_{F}G_{L}G_{F}A_{L}A_{F}U_{F18}U_{L18}G_{F}U₁₈U_{F18}A_{L}A_{F}A_{L}G_{F}C_{L}G_{L}C_{F}

### Table 18

### Application of AGSD to Murine Apo-B ags-siRNA

UUGGUAUUCAGUGUGAUGA

Haringsma et al. (Nucleic Acids Res 40: 4125-36, 2012) used this sequence to generate ssRNAi compounds against murine APO-B target site 10127 including the following:

P-U_{F}U_{F}G_{F}G_{F}U_{F}A_{F}U_{F}U_{F}C_{F}A_{F}G_{F}U_{F}G_{F}U_{F}G_{F}A_{F}U_{F}G_{F}A_{F}U_{M}U_{M}

Any of the 5′-end nucleosides (P-U_{F}) shown in the examples of agsRNAi compounds in Table 18 can be replaced with any of those provided for herein including one of the 5′end nucleosides selected from the group consisting of: P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 18 with an L modification(s) found in one or more positions 2-19 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the nucleosides in Table 18 with an L modification found in the overhang precursor can have the L replaced with any of those units and linkages provided for herein for overhang precursors including one or more of the sugar or sugar analogs independently selected from the group consisting of: F, J, W, V, Y, T, TL and I.

Any of the nucleosides in Table 18 can have any of those units and linkages provided for herein for overhang precursors.

B. Examples of Application of AGSD to this Compound:

Compositions of Matter Suitable for Use in vitro
or in a Subject with a Protective Carrier (5′-3′)

P-U_{F}U_{F3}G_{F}G_{F}U_{F3}A_{F}U_{F}U_{F}C_{F4}A_{F}G_{F}U_{F3}G_{F}U_{F}G_{F}A_{F}U_{F3}G_{F}A_{F}A_{L}G_{L}G_{F}
P-U_{F}U_{F3}G_{F}G_{F}U_{F3}A_{F}U_{F3}U_{F3}C_{F}A_{F}G_{F}U_{F}G_{F}U_{F}G_{F}A_{F}U_{F3}G_{F}A_{F}A_{L}G_{L}G_{F}
P-U_{F}U_{F3}G_{F}G_{F}U_{F3}A_{F}U_{F3}U_{F3}C_{F}A_{F}G_{F}U_{F3}G_{F}U_{F}G_{F}A_{F}U_{F3}G_{F}A_{F}A_{L}G_{L}G_{F}
P-U_{F}U_{F3}G_{F}G_{F}U_{F3}A_{F}U_{F3}U_{F3}C_{F4}A_{F}G_{F}U_{F3}G_{F}U_{F3}G_{F}A_{F}U_{F3}G_{F}A_{F}A_{L}G_{L}G_{F}
P-U_{F}U_{F12}G_{F}G_{F}U_{F12}A_{F}U_{F}U_{F}C_{F4}A_{F}G_{F}U_{F12}G_{F}U_{F}G_{F}A_{F}U_{F12}G_{F}A_{F}A_{L}G_{L}G_{F}
P-U_{F}U_{F12}G_{F}G_{F}U_{F12}A_{F}U_{F12}U_{F12}C_{F}A_{F}G_{F}U_{F}G_{F}U_{F}G_{F}A_{F}U_{F12}G_{F}A_{F}A_{L}G_{L}G_{F}
P-U_{F}U_{F12}G_{F}G_{F}U_{F12}A_{F}U_{F12}U_{F12}C_{F}A_{F}G_{F}U_{F12}G_{F}U_{F}G_{F}A_{F}U_{F12}G_{F}A_{F}A_{L}G_{L}G_{F}
P-U_{F}U_{F12}G_{F}G_{F}U_{F12}A_{F}U_{F12}U_{F12}C_{F4}A_{F}G_{F}U_{F12}G_{F}U_{F12}G_{F}A_{F}U_{F12}G_{F}A_{F}A_{L}G_{L}G_{F}
P-U_{F}U_{F18}G_{F}G_{F}U_{F18}A_{F}U_{F}U_{F}C_{F4}A_{F}G_{F}U_{F18}G_{F}U_{F}G_{F}A_{F}U_{F18}G_{F}A_{F}A_{L}G_{L}G_{F}
P-U_{F}U_{F18}G_{F}G_{F}U_{F18}A_{F}U_{F18}U_{F18}C_{F}A_{F}G_{F}U_{F}G_{F}U_{F}G_{F}A_{F}U_{F18}G_{F}A_{F}A_{L}G_{L}G_{F}
P-U_{F}U_{F18}G_{F}G_{F}U_{F18}A_{F}U_{F18}U_{F18}C_{F}A_{F}G_{F}U_{F18}G_{F}U_{F}G_{F}A_{F}U_{F18}G_{F}A_{F}A_{L}G_{L}G_{F}
P-U_{F}U_{F18}G_{F}G_{F}U_{F18}A_{F}U_{F18}U_{F18}C_{F4}A_{F}G_{F}U_{F18}G_{F}U_{F18}G_{F}A_{F}U_{F18}G_{F}A_{F}A_{L}G_{L}G_{F}
P-U_{F}U_{F3}G_{F}G_{F}U_{F3}A_{F}U_{F}U_{F}C_{F4}A_{F}G_{F}U_{F3}G_{F}UG_{F}A_{F}U_{F3}G_{F}A_{F}A_{L}G_{L}G_{F}
P-U_{F}U_{F3}G_{F}G_{F}U_{F3}A_{F}U_{F3}U_{F3}C_{F}A_{F}G_{F}U_{F}G_{F}UG_{F}A_{F}U_{F3}G_{F}A_{F}A_{L}G_{L}G_{F}
P-U_{F}U_{F3}G_{F}G_{F}U_{F3}A_{F}U_{F3}U_{F3}C_{F}A_{F}G_{F}U_{F3}G_{F}UG_{F}A_{F}U_{F3}G_{F}A_{F}A_{L}G_{L}G_{F}
P-U_{F}U_{F3}G_{F}G_{F}U_{F3}A_{F}U_{F3}U_{F3}C_{F4}A_{F}G_{F}U_{F3}G_{F}U₃G_{F}A_{F}U_{F3}G_{F}A_{F}A_{L}G_{L}G_{F}
P-U_{F}U_{F12}G_{F}G_{F}U_{F12}A_{F}U_{F}U_{F}C_{F4}A_{F}G_{F}U_{F12}G_{F}UG_{F}A_{F}U_{F12}G_{F}A_{F}A_{L}G_{L}G_{F}
P-U_{F}U_{F12}G_{F}G_{F}U_{F12}A_{F}U_{F12}U_{F12}C_{F}A_{F}G_{F}U_{F}G_{F}UG_{F}A_{F}U_{F12}G_{F}A_{F}A_{L}G_{L}G_{F}
P-U_{F}U_{F12}G_{F}G_{F}U_{F12}A_{F}U_{F12}U_{F12}C_{F}A_{F}G_{F}U_{F12}G_{F}UG_{F}A_{F}U_{F12}G_{F}A_{F}A_{L}G_{L}G_{F}
P-U_{F}U_{F12}G_{F}G_{F}U_{F12}A_{F}U_{F12}U_{F12}C_{F4}A_{F}G_{F}U_{F12}G_{F}U₁₂G_{F}A_{F}U_{F12}G_{F}A_{F}A_{L}G_{L}G_{F}
P-U_{F}U_{F18}G_{F}G_{F}U_{F18}A_{F}U_{F}U_{F}C_{F4}A_{F}G_{F}U_{F18}G_{F}UG_{F}A_{F}U_{F18}G_{F}A_{F}A_{L}G_{L}G_{F}
P-U_{F}U_{F18}G_{F}G_{F}U_{F18}A_{F}U_{F18}U_{F18}C_{F}A_{F}G_{F}U_{F}G_{F}UG_{F}A_{F}U_{F18}G_{F}A_{F}A_{L}G_{L}G_{F}
P-U_{F}U_{F18}G_{F}G_{F}U_{F18}A_{F}U_{F18}U_{F18}C_{F}A_{F}G_{F}U_{F18}G_{F}UG_{F}A_{F}U_{F18}G_{F}A_{F}A_{L}G_{L}G_{F}
P-U_{F}U_{F18}G_{F}G_{F}U_{F18}A_{F}U_{F18}U_{F18}C_{F4}A_{F}G_{F}U_{F18}G_{F}U₁₈G_{F}A_{F}U_{F18}G_{F}A_{F}A_{L}G_{L}G_{F}
P-U_{F}U_{F}G_{F}G_{L}U_{F}A_{F}U_{F}U_{F}C_{L}A_{F}G_{F}U_{L}G_{F}U_{F}G_{L}A_{F}U_{F}G_{L}A_{F}A_{L}G_{L}G_{F}
P-U_{F}U_{F}G_{F}G_{L}U_{F}A_{F}U_{F}U_{F}C_{L}A_{F}G_{F}U_{L}G_{F}UG_{L}A_{F}U_{F}G_{L}A_{F}A_{L}G_{L}G_{F}
P-U_{F}U_{F3}G_{F}G_{L}U_{F3}A_{F}U_{F}U_{F}C_{L4}A_{F}G_{F}U_{L3}G_{F}U_{F}G_{L}A_{F}U_{F3}G_{L}A_{F}A_{L}G_{L}G_{F}
P-U_{F}U_{F3}G_{F}G_{L}U_{F3}A_{F}U_{F3}U_{F3}C_{L}A_{F}G_{F}U_{L}G_{F}U_{F}G_{L}A_{F}U_{F3}G_{L}A_{F}A_{L}G_{L}G_{F}
P-U_{F}U_{F3}G_{F}G_{L}U_{F3}A_{F}U_{F3}U_{F3}C_{L}A_{F}G_{F}U_{L3}G_{F}U_{F}G_{L}A_{F}U_{F3}G_{L}A_{F}A_{L}G_{L}G_{F}
P-U_{F}U_{F3}G_{F}G_{L}U_{F3}A_{F}U_{F3}U_{F3}C_{L4}A_{F}G_{F}U_{L3}G_{F}U_{F3}G_{L}A_{F}U_{F3}G_{L}A_{F}A_{L}G_{L}G_{F}
P-U_{F}U_{F12}G_{F}G_{L}U_{F12}A_{F}U_{F}U_{F}C_{L4}A_{F}G_{F}U_{L12}G_{F}U_{F}G_{L}A_{F}U_{F12}G_{L}A_{F}A_{L}G_{L}G_{F}
P-U_{F}U_{F12}G_{F}G_{L}U_{F12}A_{F}U_{F12}U_{F12}C_{L}A_{F}G_{F}U_{L}G_{F}U_{F}G_{L}A_{F}U_{F12}G_{L}A_{F}A_{L}G_{L}G_{F}
P-U_{F}U_{F12}G_{F}G_{L}U_{F12}A_{F}U_{F12}U_{F12}C_{L}A_{F}G_{F}U_{L12}G_{F}U_{F}G_{L}A_{F}U_{F12}G_{L}A_{F}A_{L}G_{L}G_{F}
P-U_{F}U_{F12}G_{F}G_{L}U_{F12}A_{F}U_{F12}U_{F12}C_{L4}A_{F}G_{F}U_{L12}G_{F}U_{F12}G_{L}A_{F}U_{F12}G_{L}A_{F}A_{L}G_{L}G_{F}
P-U_{F}U_{F18}G_{F}G_{L}U_{F18}A_{F}U_{F}U_{F}C_{L4}A_{F}G_{F}U_{L18}G_{F}U_{F}G_{L}A_{F}U_{F18}G_{L}A_{F}A_{L}G_{L}G_{F}
P-U_{F}U_{F18}G_{F}G_{L}U_{F18}A_{F}U_{F18}U_{F18}C_{L}A_{F}G_{F}U_{L}G_{F}U_{F}G_{L}A_{F}U_{F18}G_{L}A_{F}A_{L}G_{L}G_{F}
P-U_{F}U_{F18}G_{F}G_{L}U_{F18}A_{F}U_{F18}U_{F18}C_{L}A_{F}G_{F}U_{L18}G_{F}U_{F}G_{L}A_{F}U_{F18}G_{L}A_{F}A_{L}G_{L}G_{F}
P-U_{F}U_{F18}G_{F}G_{L}U_{F18}A_{F}U_{F18}U_{F18}C_{L4}A_{F}G_{F}U_{L18}G_{F}U_{F18}G_{L}A_{F}U_{F18}G_{L}A_{F}A_{L}G_{L}G_{F}
P-U_{F}U_{F3}G_{F}G_{L}U_{F3}A_{F}U_{F}U_{F}C_{L4}A_{F}G_{F}U_{L3}G_{F}UG_{L}A_{F}U_{F3}G_{L}A_{F}A_{L}G_{L}G_{F}
P-U_{F}U_{F3}G_{F}G_{L}U_{F3}A_{F}U_{F3}U_{F3}C_{L}A_{F}G_{F}U_{L}G_{F}UG_{L}A_{F}U_{F3}G_{L}A_{F}A_{L}G_{L}G_{F}
P-U_{F}U_{F3}G_{F}G_{L}U_{F3}A_{F}U_{F3}U_{F3}C_{L}A_{F}G_{F}U_{L3}G_{F}UG_{L}A_{F}U_{F3}G_{L}A_{F}A_{L}G_{L}G_{F}
P-U_{F}U_{F3}G_{F}G_{L}U_{F3}A_{F}U_{F3}U_{F3}C_{L4}A_{F}G_{F}U_{L3}G_{F}U₃G_{L}A_{F}U_{F3}G_{L}A_{F}A_{L}G_{L}G_{F}
P-U_{F}U_{F12}G_{F}G_{L}U_{F12}A_{F}U_{F}U_{F}C_{L4}A_{F}G_{F}U_{L12}G_{F}UG_{L}A_{F}U_{F12}G_{L}A_{F}A_{L}G_{L}G_{F}
P-U_{F}U_{F12}G_{F}G_{L}U_{F12}A_{F}U_{F12}U_{F12}C_{L}A_{F}G_{F}U_{L}G_{F}UG_{L}A_{F}U_{F12}G_{L}A_{F}A_{L}G_{L}G_{F}
P-U_{F}U_{F12}G_{F}G_{L}U_{F12}A_{F}U_{F12}U_{F12}C_{L}A_{F}G_{F}U_{L12}G_{F}UG_{L}A_{F}U_{F12}G_{L}A_{F}A_{L}G_{L}G_{F}
P-U_{F}U_{F12}G_{F}G_{L}U_{F12}A_{F}U_{F12}U_{F12}C_{L4}A_{F}G_{F}U_{L12}G_{F}U₁₂G_{L}A_{F}U_{F12}G_{L}A_{F}A_{L}G_{L}G_{F}
P-U_{F}U_{F18}G_{F}G_{L}U_{F18}A_{F}U_{F}U_{F}C_{L4}A_{F}G_{F}U_{L18}G_{F}UG_{L}A_{F}U_{F18}G_{L}A_{F}A_{L}G_{L}G_{F}
P-U_{F}U_{F18}G_{F}G_{L}U_{F18}A_{F}U_{F18}U_{F18}C_{L}A_{F}G_{F}U_{L}G_{F}UG_{L}A_{F}U_{F18}G_{L}A_{F}A_{L}G_{L}G_{F}
P-U_{F}U_{F18}G_{F}G_{L}U_{F18}A_{F}U_{F18}U_{F18}C_{L}A_{F}G_{F}U_{L18}G_{F}UG_{L}A_{F}U_{F18}G_{L}A_{F}A_{L}G_{L}G_{F}
P-U_{F}U_{F18}G_{F}G_{L}U_{F18}A_{F}U_{F18}U_{F18}C_{L4}A_{F}G_{F}U_{L18}G_{F}U₁₈G_{L}A_{F}U_{F18}G_{L}A_{F}A_{L}G_{L}G_{F}
P-U_{F}U_{F}G_{L}G_{F}U_{F}A_{L}U_{F}U_{F}C_{F}A_{F}G_{F}U_{F}G_{L}U_{F}G_{F}A_{F}U_{L}G_{F}A_{L}A_{L}G_{L}G_{F}
P-U_{F}U_{F}G_{L}G_{F}U_{F}A_{L}U_{F}U_{F}C_{F}A_{F}G_{F}U_{F}G_{L}UG_{F}A_{F}U_{L}G_{F}A_{L}A_{L}G_{L}G_{F}
P-U_{F}U_{F3}G_{L}G_{F}U_{F3}A_{L}U_{F}U_{F}C_{F4}A_{F}G_{F}U_{F3}G_{L}U_{F}G_{F}A_{F}U_{L3}G_{F}A_{L}A_{L}G_{L}G_{F}
P-U_{F}U_{F3}G_{L}G_{F}U_{F3}A_{L}U_{F3}U_{F3}C_{F}A_{F}G_{F}U_{F}G_{L}U_{F}G_{F}A_{F}U_{L3}G_{F}A_{L}A_{L}G_{L}G_{F}
P-U_{F}U_{F3}G_{L}G_{F}U_{F3}A_{L}U_{F3}U_{F3}C_{F}A_{F}G_{F}U_{F3}G_{L}U_{F}G_{F}A_{F}U_{L3}G_{F}A_{L}A_{L}G_{L}G_{F}
P-U_{F}U_{F3}G_{L}G_{F}U_{F3}A_{L}U_{F3}U_{F3}C_{F4}A_{F}G_{F}U_{F3}G_{L}U_{F3}G_{F}A_{F}U_{L3}G_{F}A_{L}A_{L}G_{L}G_{F}
P-U_{F}U_{F12}G_{L}G_{F}U_{F12}A_{L}U_{F}U_{F}C_{F4}A_{F}G_{F}U_{F12}G_{L}U_{F}G_{F}A_{F}U_{L12}G_{F}A_{L}A_{L}G_{L}G_{F}
P-U_{F}U_{F12}G_{L}G_{F}U_{F12}A_{L}U_{F12}U_{F12}C_{F}A_{F}G_{F}U_{F}G_{L}U_{F}G_{F}A_{F}U_{L12}G_{F}A_{L}A_{L}G_{L}G_{F}
P-U_{F}U_{F12}G_{L}G_{F}U_{F12}A_{L}U_{F12}U_{F12}C_{F}A_{F}G_{F}U_{F12}G_{L}U_{F}G_{F}A_{F}U_{L12}G_{F}A_{L}A_{L}G_{L}G_{F}
P-U_{F}U_{F12}G_{L}G_{F}U_{F12}A_{L}U_{F12}U_{F12}C_{F4}A_{F}G_{F}U_{F12}G_{L}U_{F12}G_{F}A_{F}U_{L12}G_{F}A_{L}A_{L}G_{L}G_{F}
P-U_{F}U_{F18}G_{L}G_{F}U_{F18}A_{L}U_{F}U_{F}C_{F4}A_{F}G_{F}U_{F18}G_{L}U_{F}G_{F}A_{F}U_{L18}G_{F}A_{L}A_{L}G_{L}G_{F}
P-U_{F}U_{F18}G_{L}G_{F}U_{F18}A_{L}U_{F18}U_{F18}C_{F}A_{F}G_{F}U_{F}G_{L}U_{F}G_{F}A_{F}U_{L18}G_{F}A_{L}A_{L}G_{L}G_{F}
P-U_{F}U_{F18}G_{L}G_{F}U_{F18}A_{L}U_{F18}U_{F18}C_{F}A_{F}G_{F}U_{F18}G_{L}U_{F}G_{F}A_{F}U_{L18}G_{F}A_{L}A_{L}G_{L}G_{F}
P-U_{F}U_{F18}G_{L}G_{F}U_{F18}A_{L}U_{F18}U_{F18}C_{F4}A_{F}G_{F}U_{F18}G_{L}U_{F18}G_{F}A_{F}U_{L18}G_{F}A_{L}A_{L}G_{L}G_{F}
P-U_{F}U_{F3}G_{L}G_{F}U_{F3}A_{L}U_{F}U_{F}C_{F4}A_{F}G_{F}U_{F3}G_{L}UG_{F}A_{F}U_{L3}G_{F}A_{L}A_{L}G_{L}G_{F}
P-U_{F}U_{F3}G_{L}G_{F}U_{F3}A_{L}U_{F3}U_{F3}C_{F}A_{F}G_{F}U_{F}G_{L}UG_{F}A_{F}U_{L3}G_{F}A_{L}A_{L}G_{L}G_{F}
P-U_{F}U_{F3}G_{L}G_{F}U_{F3}A_{L}U_{F3}U_{F3}C_{F}A_{F}G_{F}U_{F3}G_{L}UG_{F}A_{F}U_{L3}G_{F}A_{L}A_{L}G_{L}G_{F}
P-U_{F}U_{F3}G_{L}G_{F}U_{F3}A_{L}U_{F3}U_{F3}C_{F4}A_{F}G_{F}U_{F3}G_{L}U₃G_{F}A_{F}U_{L3}G_{F}A_{L}A_{L}G_{L}G_{F}
P-U_{F}U_{F12}G_{L}G_{F}U_{F12}A_{L}U_{F}U_{F}C_{F4}A_{F}G_{F}U_{F12}G_{L}UG_{F}A_{F}U_{L12}G_{F}A_{L}A_{L}G_{L}G_{F}
P-U_{F}U_{F12}G_{L}G_{F}U_{F12}A_{L}U_{F12}U_{F12}C_{F}A_{F}G_{F}U_{F}G_{L}UG_{F}A_{F}U_{L12}G_{F}A_{L}A_{L}G_{L}G_{F}
P-U_{F}U_{F12}G_{L}G_{F}U_{F12}A_{L}U_{F12}U_{F12}C_{F}A_{F}G_{F}U_{F12}G_{L}UG_{F}A_{F}U_{L12}G_{F}A_{L}A_{L}G_{L}G_{F}
P-U_{F}U_{F12}G_{L}G_{F}U_{F12}A_{L}U_{F12}U_{F12}C_{F4}A_{F}G_{F}U_{F12}G_{L}U₁₂G_{F}A_{F}U_{L12}G_{F}A_{L}A_{L}G_{L}G_{F}
P-U_{F}U_{F18}G_{L}G_{F}U_{F18}A_{L}U_{F}U_{F}C_{F4}A_{F}G_{F}U_{F18}G_{L}UG_{F}A_{F}U_{L18}G_{F}A_{L}A_{L}G_{L}G_{F}
P-U_{F}U_{F18}G_{L}G_{F}U_{F18}A_{L}U_{F18}U_{F18}C_{F}A_{F}G_{F}U_{F}G_{L}UG_{F}A_{F}U_{L18}G_{F}A_{L}A_{L}G_{L}G_{F}
P-U_{F}U_{F18}G_{L}G_{F}U_{F18}A_{L}U_{F18}U_{F18}C_{F}A_{F}G_{F}U_{F18}G_{L}UG_{F}A_{F}U_{L18}G_{F}A_{L}A_{L}G_{L}G_{F}
P-U_{F}U_{F18}G_{L}G_{F}U_{F18}A_{L}U_{F18}U_{F18}C_{F4}A_{F}G_{F}U_{F18}G_{L}U₁₈G_{F}A_{F}U_{L18}G_{F}A_{L}A_{L}G_{L}G_{F}
P-U_{F}U_{F}G_{F}G_{L}U_{F}A_{F}U_{L}U_{F}C_{L}A_{F}G_{F}U_{F}G_{F}U_{F}G_{L}A_{F}U_{F}G_{L}A_{F}A_{L}G_{L}G_{F}
P-U_{F}U_{F}G_{F}G_{L}U_{F}A_{F}U_{L}U_{F}C_{L}A_{F}G_{F}U_{F}G_{F}UG_{L}A_{F}U_{F}G_{L}A_{F}A_{L}G_{L}G_{F}
P-U_{F}U_{F3}G_{F}G_{L}U_{F3}A_{F}U_{L}U_{F}C_{L4}A_{F}G_{F}U_{F3}G_{F}U_{F}G_{L}A_{F}U_{F3}G_{L}A_{F}A_{L}G_{L}G_{F}
P-U_{F}U_{F3}G_{F}G_{L}U_{F3}A_{F}U_{L3}U_{F3}C_{L}A_{F}G_{F}U_{F}G_{F}U_{F}G_{L}A_{F}U_{F3}G_{L}A_{F}A_{L}G_{L}G_{F}
P-U_{F}U_{F3}G_{F}G_{L}U_{F3}A_{F}U_{L3}U_{F3}C_{L}A_{F}G_{F}U_{F3}G_{F}U_{F}G_{L}A_{F}U_{F3}G_{L}A_{F}A_{L}G_{L}G_{F}
P-U_{F}U_{F3}G_{F}G_{L}U_{F3}A_{F}U_{L3}U_{F3}C_{L4}A_{F}G_{F}U_{F3}G_{F}U_{F3}G_{L}A_{F}U_{F3}G_{L}A_{F}A_{L}G_{L}G_{F}
P-U_{F}U_{F12}G_{F}G_{L}U_{F12}A_{F}U_{L}U_{F}C_{L4}A_{F}G_{F}U_{F12}G_{F}U_{F}G_{L}A_{F}U_{F12}G_{L}A_{F}A_{L}G_{L}G_{F}
P-U_{F}U_{F12}G_{F}G_{L}U_{F12}A_{F}U_{L12}U_{F12}C_{L}A_{F}G_{F}U_{F}G_{F}U_{F}G_{L}A_{F}U_{F12}G_{L}A_{F}A_{L}G_{L}G_{F}
P-U_{F}U_{F12}G_{F}G_{L}U_{F12}A_{F}U_{L12}U_{F12}C_{L}A_{F}G_{F}U_{F12}G_{F}U_{F}G_{L}A_{F}U_{F12}G_{L}A_{F}A_{L}G_{L}G_{F}
P-U_{F}U_{F12}G_{F}G_{L}U_{F12}A_{F}U_{L12}U_{F12}C_{L4}A_{F}G_{F}U_{F12}G_{F}U_{F12}G_{L}A_{F}U_{F12}G_{L}A_{F}A_{L}G_{L}G_{F}
P-U_{F}U_{F18}G_{F}G_{L}U_{F18}A_{F}U_{L}U_{F}C_{L4}A_{F}G_{F}U_{F18}G_{F}U_{F}G_{L}A_{F}U_{F18}G_{L}A_{F}A_{L}G_{L}G_{F}
P-U_{F}U_{F18}G_{F}G_{L}U_{F18}A_{F}U_{L18}U_{F18}C_{L}A_{F}G_{F}U_{F}G_{F}U_{F}G_{L}A_{F}U_{F18}G_{L}A_{F}A_{L}G_{L}G_{F}
P-U_{F}U_{F18}G_{F}G_{L}U_{F18}A_{F}U_{L18}U_{F18}C_{L}A_{F}G_{F}U_{F18}G_{F}U_{F}G_{L}A_{F}U_{F18}G_{L}A_{F}A_{L}G_{L}G_{F}
P-U_{F}U_{F18}G_{F}G_{L}U_{F18}A_{F}U_{L18}U_{F18}C_{L4}A_{F}G_{F}U_{F18}G_{F}U_{F18}G_{L}A_{F}U_{F18}G_{L}A_{F}A_{L}G_{L}G_{F}
P-U_{F}U_{F3}G_{F}G_{L}U_{F3}A_{F}U_{L}U_{F}C_{L4}A_{F}G_{F}U_{F3}G_{F}UG_{L}A_{F}U_{F3}G_{L}A_{F}A_{L}G_{L}G_{F}
P-U_{F}U_{F3}G_{F}G_{L}U_{F3}A_{F}U_{L3}U_{F3}C_{L}A_{F}G_{F}U_{F}G_{F}UG_{L}A_{F}U_{F3}G_{L}A_{F}A_{L}G_{L}G_{F}
P-U_{F}U_{F3}G_{F}G_{L}U_{F3}A_{F}U_{L3}U_{F3}C_{L}A_{F}G_{F}U_{F3}G_{F}UG_{L}A_{F}U_{F3}G_{L}A_{F}A_{L}G_{L}G_{F}
P-U_{F}U_{F3}G_{F}G_{L}U_{F3}A_{F}U_{L3}U_{F3}C_{L4}A_{F}G_{F}U_{F3}G_{F}U₃G_{L}A_{F}U_{F3}G_{L}A_{F}A_{L}G_{L}G_{F}
P-U_{F}U_{F12}G_{F}G_{L}U_{F12}A_{F}U_{L}U_{F}C_{L4}A_{F}G_{F}U_{F12}G_{F}UG_{L}A_{F}U_{F12}G_{L}A_{F}A_{L}G_{L}G_{F}
P-U_{F}U_{F12}G_{F}G_{L}U_{F12}A_{F}U_{L12}U_{F12}C_{L}A_{F}G_{F}U_{F}G_{F}UG_{L}A_{F}U_{F12}G_{L}A_{F}A_{L}G_{L}G_{F}
P-U_{F}U_{F12}G_{F}G_{L}U_{F12}A_{F}U_{L12}U_{F12}CIA_{F}G_{F}U_{F12}G_{F}UG_{L}A_{F}U_{F12}G_{L}A_{F}A_{L}G_{L}G_{F}
P-U_{F}U_{F12}G_{F}G_{L}U_{F12}A_{F}U_{L12}U_{F12}C_{L4}A_{F}G_{F}U_{F12}G_{F}U₁₂G_{L}A_{F}U_{F12}G_{L}A_{F}A_{L}G_{L}G_{F}
P-U_{F}U_{F18}G_{F}G_{L}U_{F18}A_{F}U_{L}U_{F}C_{L4}A_{F}G_{F}U_{F18}G_{F}UG_{L}A_{F}U_{F18}G_{L}A_{F}A_{L}G_{L}G_{F}
P-U_{F}U_{F18}G_{F}G_{L}U_{F18}A_{F}U_{L18}U_{F18}C_{L}A_{F}G_{F}U_{F}G_{F}UG_{L}A_{F}U_{F18}G_{L}A_{F}A_{L}G_{L}G_{F}
P-U_{F}U_{F18}G_{F}G_{L}U_{F18}A_{F}U_{L18}U_{F18}C_{L}A_{F}G_{F}U_{F18}G_{F}UG_{L}A_{F}U_{F18}G_{L}A_{F}A_{L}G_{L}G_{F}
P-U_{F}U_{F18}G_{F}G_{L}U_{F18}A_{F}U_{L18}U_{F18}C_{L4}A_{F}G_{F}U_{F18}G_{F}U₁₈G_{L}A_{F}U_{F18}G_{L}A_{F}A_{L}G_{L}G_{F}
P-U_{F}U_{F}G_{L}G_{F}U_{F}A_{L}U_{F}U_{L}C_{F}A_{F}G_{F}U_{L}G_{F}U_{F}G_{F}A_{L}U_{F}G_{L}A_{F}A_{L}G_{L}G_{F}
P-U_{F}U_{F}G_{L}G_{F}U_{F}A_{L}U_{F}U_{L}C_{F}A_{F}G_{F}U_{L}G_{F}UG_{F}A_{L}U_{F}G_{L}A_{F}A_{L}G_{L}G_{F}
P-U_{F}U_{F3}G_{L}G_{F}U_{F3}A_{L}U_{F}U_{L}C_{F4}A_{F}G_{F}U_{L3}G_{F}U_{F}G_{F}A_{L}U_{F3}G_{L}A_{F}A_{L}G_{L}G_{F}
P-U_{F}U_{F3}G_{L}G_{F}U_{F3}A_{L}U_{F3}U_{L3}C_{F}A_{F}G_{F}U_{L}G_{F}U_{F}G_{F}A_{L}U_{F3}G_{L}A_{F}A_{L}G_{L}G_{F}
P-U_{F}U_{F3}G_{L}G_{F}U_{F3}A_{L}U_{F3}U_{L3}C_{F}A_{F}G_{F}U_{L3}G_{F}U_{F}G_{F}A_{L}U_{F3}G_{L}A_{F}A_{L}G_{L}G_{F}
P-U_{F}U_{F3}G_{L}G_{F}U_{F3}A_{L}U_{F3}U_{L3}C_{F4}A_{F}G_{F}U_{L3}G_{F}U_{F3}G_{F}A_{L}U_{F3}G_{L}A_{F}A_{L}G_{L}G_{F}
P-U_{F}U_{F12}G_{L}G_{F}U_{F12}A_{L}U_{F}U_{L}C_{F4}A_{F}G_{F}U_{L12}G_{F}U_{F}G_{F}A_{L}U_{F12}G_{L}A_{F}A_{L}G_{L}G_{F}
P-U_{F}U_{F12}G_{L}G_{F}U_{F12}A_{L}U_{F12}U_{L12}C_{F}A_{F}G_{F}U_{L}G_{F}U_{F}G_{F}A_{L}U_{F12}G_{L}A_{F}A_{L}G_{L}G_{F}
P-U_{F}U_{F12}G_{L}G_{F}U_{F12}A_{L}U_{F12}U_{L12}C_{F}A_{F}G_{F}U_{L12}G_{F}U_{F}G_{F}A_{L}U_{F12}G_{L}A_{F}A_{L}G_{L}G_{F}
P-U_{F}U_{F12}G_{L}G_{F}U_{F12}A_{L}U_{F12}U_{L12}C_{F4}A_{F}G_{F}U_{L12}G_{F}U_{F12}G_{F}A_{L}U_{F12}G_{L}A_{F}A_{L}G_{L}G_{F}
P-U_{F}U_{F18}G_{L}G_{F}U_{F18}A_{L}U_{F}U_{L}C_{F4}A_{F}G_{F}U_{L18}G_{F}U_{F}G_{F}A_{L}U_{F18}G_{L}A_{F}A_{L}G_{L}G_{F}
P-U_{F}U_{F18}G_{L}G_{F}U_{F18}A_{L}U_{F18}U_{L18}C_{F}A_{F}G_{F}U_{L}G_{F}U_{F}G_{F}A_{L}U_{F18}G_{L}A_{F}A_{L}G_{L}G_{F}
P-U_{F}U_{F18}G_{L}G_{F}U_{F18}A_{L}U_{F18}U_{L18}C_{F}A_{F}G_{F}U_{L18}G_{F}U_{F}G_{F}A_{L}U_{F18}G_{L}A_{F}A_{L}G_{L}G_{F}
P-U_{F}U_{F18}G_{L}G_{F}U_{F18}A_{L}U_{F18}U_{L18}C_{F4}A_{F}G_{F}U_{L18}G_{F}U_{F18}G_{F}A_{L}U_{F18}G_{L}A_{F}A_{L}G_{L}G_{F}
P-U_{F}U_{F3}G_{L}G_{F}U_{F3}A_{L}U_{F}U_{L}C_{F4}A_{F}G_{F}U_{L3}G_{F}UG_{F}A_{L}U_{F3}G_{L}A_{F}A_{L}G_{L}G_{F}
P-U_{F}U_{F3}G_{L}G_{F}U_{F3}A_{L}U_{F3}U_{L3}C_{F}A_{F}G_{F}U_{L}G_{F}UG_{F}A_{L}U_{F3}G_{L}A_{F}A_{L}G_{L}G_{F}
P-U_{F}U_{F3}G_{L}G_{F}U_{F3}A_{L}U_{F3}U_{L3}C_{F}A_{F}G_{F}U_{L3}G_{F}UG_{F}A_{L}U_{F3}G_{L}A_{F}A_{L}G_{L}G_{F}
P-U_{F}U_{F3}G_{L}G_{F}U_{F3}A_{L}U_{F3}U_{L3}C_{F4}A_{F}G_{F}U_{L3}G_{F}U₃G_{F}A_{L}U_{F3}G_{L}A_{F}A_{L}G_{L}G_{F}
P-U_{F}U_{F12}G_{L}G_{F}U_{F12}A_{L}U_{F}U_{L}C_{F4}A_{F}G_{F}U_{L12}G_{F}UG_{F}A_{L}U_{F12}G_{L}A_{F}A_{L}G_{L}G_{F}
P-U_{F}U_{F12}G_{L}G_{F}U_{F12}A_{L}U_{F12}U_{L12}C_{F}A_{F}G_{F}U_{L}G_{F}UG_{F}A_{L}U_{F12}G_{L}A_{F}A_{L}G_{L}G_{F}
P-U_{F}U_{F12}G_{L}G_{F}U_{F12}A_{L}U_{F12}U_{L12}C_{F}A_{F}G_{F}U_{L12}G_{F}UG_{F}A_{L}U_{F12}G_{L}A_{F}A_{L}G_{L}G_{F}
P-U_{F}U_{F12}G_{L}G_{F}U_{F12}A_{L}U_{F12}U_{L12}C_{F4}A_{F}G_{F}U_{L12}G_{F}U₁₂G_{F}A_{L}U_{F12}G_{L}A_{F}A_{L}G_{L}G_{F}
P-U_{F}U_{F18}G_{L}G_{F}U_{F18}A_{L}U_{F}U_{L}C_{F4}A_{F}G_{F}U_{L18}G_{F}UG_{F}A_{L}U_{F18}G_{L}A_{F}A_{L}G_{L}G_{F}
P-U_{F}U_{F18}G_{L}G_{F}U_{F18}A_{L}U_{F18}U_{L18}C_{F}A_{F}G_{F}U_{L}G_{F}UG_{F}A_{L}U_{F18}G_{L}A_{F}A_{L}G_{L}G_{F}
P-U_{F}U_{F18}G_{L}G_{F}U_{F18}A_{L}U_{F18}U_{L18}C_{F}A_{F}G_{F}U_{L18}G_{F}UG_{F}A_{L}U_{F18}G_{L}A_{F}A_{L}G_{L}G_{F}
P-U_{F}U_{F18}G_{L}G_{F}U_{F18}A_{L}U_{F18}U_{L18}C_{F4}A_{F}G_{F}U_{L18}G_{F}U₁₈G_{F}A_{L}U_{F18}G_{L}A_{F}A_{L}G_{L}G_{F}

### Table 19

### Examples of Seven Seed Vehicles Suitable for Use with a 5′-End Module Consisting of the Nine 5′-End Nucleosides of any MiRNA Guide Stand

### A. Seed Vehicles Designed and Evaluated by Chorn et al., RNA 18: 1796-804, 2012.

AGSD can be applied to any ss-RNAi known in the art to improve its activity, potency and/or duration of effect. In the case of ags-MiRs that use their seed sequence to find their targets, however, the modular approach can be used as an alternative to modifying the endogenous miRNA guide strand to be mimicked.

The modular approach involves the use of a 5′-end module that is an 8-mer or 9-mer that includes the seed sequence of the miRNA guide strand to be mimicked (or a novel seed sequence) along with a seed vehicle and typically an overhang precursor. Seed vehicles are 11 or 10-mers depending on the length of the 5′-end module to produce a 19-mer exclusive of any overhang precursor. A seed vehicle can have any RNA sequence comprising the standard bases (A, U, G and C), but in order to demonstrate the advantages of ags-MiRs over the best ss-RNAi miRNA mimics known in the art the examples found in Tables 20 and 21 make use of the best of the known seed vehicle sequences after they have undergone AGSD modifications.

The best seed vehicles come from Chorn et al. (RNA 18: 1796-804, 2012). They evaluated 88 miRNA-124 mimics constructed using the modular approach. The 5′-end module (positions 1-9) (UAAGGCACG) contained the miR-124 seed sequence and the seed vehicles (positions 10-19) had randomly selected sequences. All the linkages in these compounds were phosphodiester and all the nucleosides had the 2′-fluoro modification in nucleoside positions 1-19. In addition all of the compounds had a U_{M}U_{M} overhang precursor.

These 88 compounds were tested for activity following transfection of HCT-116 cells grown in culture. Of these 88 compounds 7 (7y_4, 8y_3, 8y_4, 9y_2, 9y_3, 10y_1 and 10y_7) had the highest levels of activity. Their activity levels, however, were not statistically significantly different. These seed vehicles are shown in Table 19A and are designated by the name of the Chorn et al. compound in which they are found.

TABLE 19A
Seed Vehicles Used by Chorn et al. (2012) that
Provided the Highest ss-RNAi miRNA Mimic
Activity

| Seed Vehicle | |
|---|---|
| Designation | Modified Seed Vehicle Sequence |
| 7y_4 | G_{F}U_{F}C_{F}U_{F}U_{F}A_{F}U_{F}U_{F}U_{F}G_{F} |
| 8y_3 | U_{F}G_{F}C_{F}U_{F}C_{F}C_{F}C_{F}A_{F}C_{F}C_{F} |
| 8y_4 | C_{F}C_{F}C_{F}U_{F}C_{F}C_{F}C_{F}U_{F}G_{F}A_{F} |
| 9y_2 | C_{F}G_{F}C_{F}U_{F}U_{F}C_{F}U_{F}C_{F}U_{F}U_{F} |
| 9y_3 | U_{F}U_{F}C_{F}U_{F}C_{F}G_{F}U_{F}C_{F}U_{F}C_{F} |
| 10y_1 | U_{F}C_{F}C_{F}C_{F}U_{F}U_{F}C_{F}U_{F}U_{F}C_{F} |
| 10y_7 | C_{F}U_{F}C_{F}C_{F}C_{F}U_{F}U_{F}U_{F}C_{F}U_{F} |

B. Application of AGSD to these Modular Components and Compounds:

Tables 19B and 19C provide examples of the seed vehicles shown in Table 19A that have been modified by AGSD. Table 19B uses the same level of basic nuclease resistance for the seed sequence as was used by Chorn et al. which requires any ags-MIR based on them to be used with a protective carrier when treating subjects. The examples in Table 19B have a higher level of basic nuclease resistance suitable for use in ags-MiRs to be used with or without a protective carrier when treating subjects.

Any of the nucleosides in Tables 19B or 19C with an L modification found in one or more nucleoside positions 10-19 can have any given L or set of Ls replaced with any of those sugar, sugar analogs or sugar substitutes provided for herein for positions 2-19 including one or more of those independently selected from the group consisting of S, J, W, V, Q, Y, O, T and cet (i.e., if multiple Ls are replaced the selected replacements do not all have to be the same sugar, sugar analog or sugar substitute).

Any of the examples of seed vehicles shown in Table 19B or 19C can have any of the overhang precursors provided for herein.

TABLE 19B
Examples of the Application of AGSD to the Seed
Vehicles used by Chorn et al. (2012)

| | Compositions of Matter Suitable |
|---|---|
| Seed Vehicle | for Use in vitro or in a Subject with |
| Designation | a Protective Carrier (5′-3′) |
| 7y_4 | G_{F}U_{F}C_{F}U_{F3}U_{F}A_{F}U_{F}U_{F3}U_{F3}G_{F} |
| | G_{F}U_{F3}C_{F}U_{F3}U_{F}A_{F}U_{F3}U_{F3}U_{F3}G_{F} |
| | G_{F}U_{F3}C_{F}U_{F3}U_{F3}A_{F}U_{F3}U_{F3}U_{F}G_{F} |
| | G_{F}U_{F}C_{F4}U_{F}U_{F}A_{F}U_{F3}U_{F3}U_{F3}G_{F} |
| | G_{F}U_{F}C_{F}U_{F3}UA_{F}U_{F}U_{F3}U_{F3}G_{F} |
| | G_{F}U_{F3}C_{F}U_{F3}UA_{F}U_{F3}U_{F3}U_{F3}G_{F} |
| | G_{F}U_{F3}C_{F}U_{F3}U₃A_{F}U_{F3}U_{F3}U_{F}G_{F} |
| | G_{F}U_{F}C_{F4}U_{F}UA_{F}U_{F3}U_{F3}U_{F3}G_{F} |
| | G_{F}U_{F}C_{F}U_{F12}U_{F}A_{F}U_{F}U_{F12}U_{F12}G_{F} |
| | G_{F}U_{F12}C_{F}U_{F12}U_{F}A_{F}U_{F12}U_{F12}U_{F12}G_{F} |
| | G_{F}U_{F12}C_{F}U_{F12}U_{F12}A_{F}U_{F12}U_{F12}U_{F}G_{F} |
| | G_{F}U_{F}C_{F4}U_{F}U_{F}A_{F}U_{F12}U_{F12}U_{F12}G_{F} |
| | G_{F}U_{F}C_{F}U_{F12}UA_{F}U_{F}U_{F12}U_{F12}G_{F} |
| | G_{F}U_{F12}C_{F}U_{F12}UA_{F}U_{F12}U_{F12}U_{F12}G_{F} |
| | G_{F}U_{F12}C_{F}U_{F12}U₁₂A_{F}U_{F12}U_{F12}U_{F}G_{F} |
| | G_{F}U_{F}C_{F4}U_{F}UA_{F}U_{F12}U_{F12}U_{F12}G_{F} |
| | G_{F}U_{F}C_{F}U_{F18}U_{F}A_{F}U_{F}U_{F18}U_{F18}G_{F} |
| | G_{F}U_{F18}C_{F}U_{F18}U_{F}A_{F}U_{F18}U_{F18}U_{F18}G_{F} |
| | G_{F}U_{F18}C_{F}U_{F18}U_{F18}A_{F}U_{F18}U_{F18}U_{F}G_{F} |
| | G_{F}U_{F}C_{F4}U_{F}U_{F}A_{F}U_{F18}U_{F18}U_{F18}G_{F} |
| | G_{F}U_{F}C_{F}U_{F18}UA_{F}U_{F}U_{F18}U_{F18}G_{F} |
| | G_{F}U_{F18}C_{F}U_{F18}UA_{F}U_{F18}U_{F18}U_{F18}G_{F} |
| | G_{F}U_{F18}C_{F}U_{F18}U₁₈A_{F}U_{F18}U_{F18}U_{F}G_{F} |
| | G_{F}U_{F}C_{F4}U_{F}UA_{F}U_{F18}U_{F18}U_{F18}G_{F} |
| | G_{F}U_{L}C_{F}U_{F}U_{F}A_{L}U_{F}U_{F}U_{L}G_{F} |
| | G_{F}U_{L}C_{F}U_{F}UA_{L}U_{F}U_{F}U_{L}G_{F} |
| | G_{F}U_{L}C_{F}U_{F3}U_{F}A_{L}U_{F}U_{F3}U_{L3}G_{F} |
| | G_{F}U_{L3}C_{F}U_{F3}U_{F}A_{L}U_{F3}U_{F3}U_{L3}G_{F} |
| | G_{F}U_{L3}C_{F}U_{F3}U_{F3}A_{L}U_{F3}U_{F3}U_{L}G_{F} |
| | G_{F}U_{L}C_{F4}U_{F}U_{F}A_{L}U_{F3}U_{F3}U_{L3}G_{F} |
| | G_{F}U_{L}C_{F}U_{F3}UA_{L}U_{F}U_{F3}U_{L3}G_{F} |
| | G_{F}U_{L3}C_{F}U_{F3}UA_{L}U_{F3}U_{F3}U_{L3}G_{F} |
| | G_{F}U_{L3}C_{F}U_{F3}U₃A_{L}U_{F3}U_{F3}U_{L}G_{F} |
| | G_{F}U_{L}C_{F4}U_{F}UA_{L}U_{F3}U_{F3}U_{L3}G_{F} |
| | G_{F}U_{L}C_{F}U_{F12}U_{F}A_{L}U_{F}U_{F12}U_{L12}G_{F} |
| | G_{F}U_{L12}C_{F}U_{F12}U_{F}A_{L}U_{F12}U_{F12}U_{L12}G_{F} |
| | G_{F}U_{L12}C_{F}U_{F12}U_{F12}A_{L}U_{F12}U_{F12}U_{L}G_{F} |
| | G_{F}U_{L}C_{F4}U_{F}U_{F}A_{L}U_{F12}U_{F12}U_{L12}G_{F} |
| | G_{F}U_{L}C_{F}U_{F12}UA_{L}U_{F}U_{F12}U_{L12}G_{F} |
| | G_{F}U_{L12}C_{F}U_{F12}UA_{L}U_{F12}U_{F12}U_{L12}G_{F} |
| | G_{F}U_{L12}C_{F}U_{F12}U₁₂A_{L}U_{F12}U_{F12}U_{L}G_{F} |
| | G_{F}U_{L}C_{F4}U_{F}UA_{L}U_{F12}U_{F12}U_{L12}G_{F} |
| | G_{F}U_{L}C_{F}U_{F18}U_{F}A_{L}U_{F}U_{F18}U_{L18}G_{F} |
| | G_{F}U_{L18}C_{F}U_{F18}U_{F}A_{L}U_{F18}U_{F18}U_{L18}G_{F} |
| | G_{F}U_{L18}C_{F}U_{F18}U_{F18}A_{L}U_{F18}U_{F18}U_{L}G_{F} |
| | G_{F}U_{L}C_{F4}U_{F}U_{F}A_{L}U_{F18}U_{F18}U_{L18}G_{F} |
| | G_{F}U_{L}C_{F}U_{F18}UA_{L}U_{F}U_{F18}U_{L18}G_{F} |
| | G_{F}U_{L18}C_{F}U_{F18}UA_{L}U_{F18}U_{F18}U_{L18}G_{F} |
| | G_{F}U_{L18}C_{F}U_{F18}U₁₈A_{L}U_{F18}U_{F18}U_{L}G_{F} |
| | G_{F}U_{L}C_{F4}U_{F}UA_{L}U_{F18}U_{F18}U_{L18}G_{F} |
| | G_{L}U_{F}C_{F}U_{L}U_{F}A_{F}U_{F}U_{L}U_{F}G_{F} |
| | G_{L}U_{F}C_{F}U_{L}UA_{F}U_{F}U_{L}U_{F}G_{F} |
| | G_{L}U_{F}C_{F}U_{L3}U_{F}A_{F}U_{F}U_{L3}U_{F3}G_{F} |
| | G_{L}U_{F3}C_{F}U_{L3}U_{F}A_{F}U_{F3}U_{L3}U_{F3}G_{F} |
| | G_{L}U_{F3}C_{F}U_{L3}U_{F3}A_{F}U_{F3}U_{L3}U_{F}G_{F} |
| | G_{L}U_{F}C_{F4}U_{L}U_{F}A_{F}U_{F3}U_{L3}U_{F3}G_{F} |
| | G_{L}U_{F}C_{F}U_{L3}UA_{F}U_{F}U_{L3}U_{F3}G_{F} |
| | G_{L}U_{F3}C_{F}U_{L3}UA_{F}U_{F3}U_{L3}U_{F3}G_{F} |
| | G_{L}U_{F3}C_{F}U_{L3}U₃A_{F}U_{F3}U_{L3}U_{F}G_{F} |
| | G_{L}U_{F}C_{F4}U_{L}UA_{F}U_{F3}U_{L3}U_{F3}G_{F} |
| | G_{L}U_{F}C_{F}U_{L12}U_{F}A_{F}U_{F}U_{L12}U_{F12}G_{F} |
| | G_{L}U_{F12}C_{F}U_{L12}U_{F}A_{F}U_{F12}U_{L12}U_{F12}G_{F} |
| | G_{L}U_{F12}C_{F}U_{L12}U_{F12}A_{F}U_{F12}U_{L12}U_{F}G_{F} |
| | G_{L}U_{F}C_{F4}U_{L}U_{F}A_{F}U_{F12}U_{L12}U_{F12}G_{F} |
| | G_{L}U_{F}C_{F}U_{L12}UA_{F}U_{F}U_{L12}U_{F12}G_{F} |
| | G_{L}U_{F12}C_{F}U_{L12}UA_{F}U_{F12}U_{L12}U_{F12}G_{F} |
| | G_{L}U_{F12}C_{F}U_{L12}U₁₂A_{F}U_{F12}U_{L12}U_{F}G_{F} |
| | G_{L}U_{F}C_{F4}U_{L}UA_{F}U_{F12}U_{L12}U_{F12}G_{F} |
| | G_{L}U_{F}C_{F}U_{L18}U_{F}A_{F}U_{F}U_{L18}U_{F18}G_{F} |
| | G_{L}U_{F18}C_{F}U_{L18}U_{F}A_{F}U_{F18}U_{L18}U_{F18}G_{F} |
| | G_{L}U_{F18}C_{F}U_{L18}U_{F18}A_{F}U_{F18}U_{L18}U_{F}G_{F} |
| | G_{L}U_{F}C_{F4}U_{L}U_{F}A_{F}U_{F18}U_{L18}U_{F18}G_{F} |
| | G_{L}U_{F}C_{F}U_{L18}UA_{F}U_{F}U_{L18}U_{F18}G_{F} |
| | G_{L}U_{F18}C_{F}U_{L18}UA_{F}U_{F18}U_{L18}U_{F18}G_{F} |
| | G_{L}U_{F18}C_{F}U_{L18}U₁₈A_{F}U_{F18}U_{L18}U_{F}G_{F} |
| | G_{L}U_{F}C_{F4}U_{L}UA_{F}U_{F18}U_{L18}U_{F18}G_{F} |
| | G_{F}U_{F}C_{L}U_{F}U_{F}A_{L}U_{F}U_{F}U_{L}G_{F} |
| | G_{F}U_{F}C_{L}U_{F}UA_{L}U_{F}U_{F}U_{L}G_{F} |
| | G_{F}U_{F}C_{L}U_{F3}U_{F}A_{L}U_{F}U_{F3}U_{L3}G_{F} |
| | G_{F}U_{F3}C_{L}U_{F3}U_{F}A_{L}U_{F3}U_{F3}U_{L3}G_{F} |
| | G_{F}U_{F3}C_{L}U_{F3}U_{F3}A_{L}U_{F3}U_{F3}U_{L}G_{F} |
| | G_{F}U_{F}C_{L4}U_{F}U_{F}A_{L}U_{F3}U_{F3}U_{L3}G_{F} |
| | G_{F}U_{F}C_{L}U_{F3}UA_{L}U_{F}U_{F3}U_{L3}G_{F} |
| | G_{F}U_{F3}C_{L}U_{F3}UA_{L}U_{F3}U_{F3}U_{L3}G_{F} |
| | G_{F}U_{F3}C_{L}U_{F3}U₃A_{L}U_{F3}U_{F3}U_{L}G_{F} |
| | G_{F}U_{F}C_{L4}U_{F}UA_{L}U_{F3}U_{F3}U_{L3}G_{F} |
| | G_{F}U_{F}C_{L}U_{F12}U_{F}A_{L}U_{F}U_{F12}U_{L12}G_{F} |
| | G_{F}U_{F12}C_{L}U_{F12}U_{F}A_{L}U_{F12}U_{F12}U_{L12}G_{F} |
| | G_{F}U_{F12}C_{L}U_{F12}U_{F12}A_{L}U_{F12}U_{F12}U_{L}G_{F} |
| | G_{F}U_{F}C_{L4}U_{F}U_{F}A_{L}U_{F12}U_{F12}U_{L12}G_{F} |
| | G_{F}U_{F}C_{L}U_{F12}UA_{L}U_{F}U_{F12}U_{L12}G_{F} |
| | G_{F}U_{F12}C_{L}U_{F12}UA_{L}U_{F12}U_{F12}U_{L12}G_{F} |
| | G_{F}U_{F12}C_{L}U_{F12}U₁₂A_{L}U_{F12}U_{F12}U_{L}G_{F} |
| | G_{F}U_{F}C_{L4}U_{F}UA_{L}U_{F12}U_{F12}U_{L12}G_{F} |
| | G_{F}U_{F}C_{L}U_{F18}U_{F}A_{L}U_{F}U_{F18}U_{L18}G_{F} |
| | G_{F}U_{F18}C_{L}U_{F18}U_{F}A_{L}U_{F18}U_{F18}U_{L18}G_{F} |
| | G_{F}U_{F18}C_{L}U_{F18}U_{F18}A_{L}U_{F18}U_{F18}U_{L}G_{F} |
| | G_{F}U_{F}C_{L4}U_{F}U_{F}A_{L}U_{F18}U_{F18}U_{L18}G_{F} |
| | G_{F}U_{F}C_{L}U_{F18}UA_{L}U_{F}U_{F18}U_{L18}G_{F} |
| | G_{F}U_{F18}C_{L}U_{F18}UA_{L}U_{F18}U_{F18}U_{L18}G_{F} |
| | G_{F}U_{F18}C_{L}U_{F18}U₁₈A_{L}U_{F18}U_{F18}U_{L}G_{F} |
| | G_{F}U_{F}C_{L4}U_{F}UA_{L}U_{F18}U_{F18}U_{L18}G_{F} |
| | G_{F}U_{L}C_{F}U_{L}U_{F}A_{F}U_{F}U_{L}U_{F}G_{L} |
| | G_{F}U_{L}C_{F}U_{L}UA_{F}U_{F}U_{L}U_{F}G_{L} |
| | G_{F}U_{L}C_{F}U_{L3}U_{F}A_{F}U_{F}U_{L3}U_{F3}G_{L} |
| | G_{F}U_{L3}C_{F}U_{L3}U_{F}A_{F}U_{F3}U_{L3}U_{F3}G_{L} |
| | G_{F}U_{L3}C_{F}U_{L3}U_{F3}A_{F}U_{F3}U_{L3}U_{F}G_{L} |
| | G_{F}U_{L}C_{F4}U_{L}U_{F}A_{F}U_{F3}U_{L3}U_{F3}G_{L} |
| | G_{F}U_{L}C_{F}U_{L3}UA_{F}U_{F}U_{L3}U_{F3}G_{L} |
| | G_{F}U_{L3}C_{F}U_{L3}UA_{F}U_{F3}U_{L3}U_{F3}G_{L} |
| | G_{F}U_{L3}C_{F}U_{L3}U₃A_{F}U_{F3}U_{L3}U_{F}G_{L} |
| | G_{F}U_{L}C_{F4}U_{L}UA_{F}U_{F3}U_{L3}U_{F3}G_{L} |
| | G_{F}U_{L}C_{F}U_{L12}U_{F}A_{F}U_{F}U_{L12}U_{F12}G_{L} |
| | G_{F}U_{L12}C_{F}U_{L12}U_{F}A_{F}U_{F12}U_{L12}U_{F12}G_{L} |
| | G_{F}U_{L12}C_{F}U_{L12}U_{F12}A_{F}U_{F12}U_{L12}U_{F}G_{L} |
| | G_{F}U_{L}C_{F4}U_{L}U_{F}A_{F}U_{F12}U_{L12}U_{F12}G_{L} |
| | G_{F}U_{L}C_{F}U_{L12}UA_{F}U_{F}U_{L12}U_{F12}G_{L} |
| | G_{F}U_{L12}C_{F}U_{L12}UA_{F}U_{F12}U_{L12}U_{F12}G_{L} |
| | G_{F}U_{L12}C_{F}U_{L12}U₁₂A_{F}U_{F12}U_{L12}U_{F}G_{L} |
| | G_{F}U_{L}C_{F4}U_{L}UA_{F}U_{F12}U_{L12}U_{F12}G_{L} |
| | G_{F}U_{L}C_{F}U_{L18}U_{F}A_{F}U_{F}U_{L18}U_{F18}G_{L} |
| | G_{F}U_{L18}C_{F}U_{L18}U_{F}A_{F}U_{F18}U_{L18}U_{F18}G_{L} |
| | G_{F}U_{L18}C_{F}U_{L18}U_{F18}A_{F}U_{F18}U_{L18}U_{F}G_{L} |
| | G_{F}U_{L}C_{F4}U_{L}U_{F}A_{F}U_{F18}U_{L18}U_{F18}G_{L} |
| | G_{F}U_{L}C_{F}U_{L18}UA_{F}U_{F}U_{L18}U_{F18}G_{L} |
| | G_{F}U_{L18}C_{F}U_{L18}UA_{F}U_{F18}U_{L18}U_{F18}G_{L} |
| | G_{F}U_{L18}C_{F}U_{L18}U₁₈A_{F}U_{F18}U_{L18}U_{F}G_{L} |
| | G_{F}U_{L}C_{F4}U_{L}UA_{F}U_{F18}U_{L18}U_{F18}G_{L} |
| 8y_3 | U_{F3}G_{F}C_{F4}U_{F}C_{F}C_{F4}C_{F}A_{F}C_{F4}C_{F} |
| | U_{F3}G_{F}C_{F}U_{F3}C_{F}C_{F}C_{F4}A_{F}C_{F4}C_{F} |
| | U_{F}G_{F}C_{F4}U_{F}C_{F}C_{F4}C_{F}A_{F}C_{F4}C_{F4} |
| | U_{F3}G_{F}C_{F4}U_{F}C_{F}C_{F4}C_{F4}A_{F}C_{F4}C_{F4} |
| | U_{F3}G_{F}C_{F4}U_{F}CC_{F4}C_{F}A_{F}C_{F4}C_{F} |
| | U_{F3}G_{F}C_{F}U_{F3}CC_{F}C_{F4}A_{F}C_{F4}C_{F} |
| | U_{F}G_{F}C_{F4}U_{F}CC_{F4}C_{F}A_{F}C_{F4}C_{F4} |
| | U_{F3}G_{F}C_{F4}U_{F}CC_{F4}C_{F4}A_{F}C_{F4}C_{F4} |
| | U_{F12}G_{F}C_{F4}U_{F}C_{F}C_{F4}C_{F}A_{F}C_{F4}C_{F} |
| | U_{F12}G_{F}C_{F}U_{F12}C_{F}C_{F}C_{F4}A_{F}C_{F4}C_{F} |
| | U_{F12}G_{F}C_{F4}U_{F}C_{F}C_{F4}C_{F4}A_{F}C_{F4}C_{F4} |
| | U_{F12}G_{F}C_{F4}U_{F}CC_{F4}C_{F}A_{F}C_{F4}C_{F} |
| | U_{F12}G_{F}C_{F}U_{F12}CC_{F}C_{F4}A_{F}C_{F4}C_{F} |
| | U_{F12}G_{F}C_{F4}U_{F}CC_{F4}C_{F4}A_{F}C_{F4}C_{F4} |
| | U_{F18}G_{F}C_{F4}U_{F}C_{F}C_{F4}C_{F}A_{F}C_{F4}C_{F} |
| | U_{F18}G_{F}C_{F}U_{F18}C_{F}C_{F}C_{F4}A_{F}C_{F4}C_{F} |
| | U_{F18}G_{F}C_{F4}U_{F}C_{F}C_{F4}C_{F4}A_{F}C_{F4}C_{F4} |
| | U_{F18}G_{F}C_{F4}U_{F}CC_{F4}C_{F}A_{F}C_{F4}C_{F} |
| | U_{F18}G_{F}C_{F}U_{F18}CC_{F}C_{F4}A_{F}C_{F4}C_{F} |
| | U_{F18}G_{F}C_{F4}U_{F}CC_{F4}C_{F4}A_{F}C_{F4}C_{F4} |
| | U_{F}G_{L}C_{F}U_{F}C_{F}C_{L}C_{F}A_{L}C_{F}C_{F} |
| | U_{F}G_{L}C_{F}U_{F}C_{F}C_{L}C_{F}A_{L}C_{F}C_{F} |
| | U_{F3}G_{L}C_{F4}U_{F}C_{F}C_{L4}C_{F}A_{L}C_{F4}C_{F} |
| | U_{F3}G_{L}C_{F}U_{F3}C_{F}C_{L}C_{F4}A_{L}C_{F4}C_{F} |
| | U_{F}G_{L}C_{F4}U_{F}C_{F}C_{L4}C_{F}A_{L}C_{F4}C_{F4} |
| | U_{F3}G_{L}C_{F4}U_{F}C_{F}C_{L4}C_{F4}A_{L}C_{F4}C_{F4} |
| | U_{F3}G_{L}C_{F4}U_{F}CC_{L4}C_{F}A_{L}C_{F4}C_{F} |
| | U_{F3}G_{L}C_{F}U_{F3}CC_{L}C_{F4}A_{L}C_{F4}C_{F} |
| | U_{F}G_{L}C_{F4}U_{F}CC_{L4}C_{F}A_{L}C_{F4}C_{F4} |
| | U_{F3}G_{L}C_{F4}U_{F}CC_{L4}C_{F4}A_{L}C_{F4}C_{F4} |
| | U_{F12}G_{L}C_{F4}U_{F}C_{F}C_{L4}C_{F}A_{L}C_{F4}C_{F} |
| | U_{F12}G_{L}C_{F}U_{F12}C_{F}C_{L}C_{F4}A_{L}C_{F4}C_{F} |
| | U_{F12}G_{L}C_{F4}U_{F}C_{F}C_{L4}C_{F4}A_{L}C_{F4}C_{F4} |
| | U_{F12}G_{L}C_{F4}U_{F}CC_{L4}C_{F}A_{L}C_{F4}C_{F} |
| | U_{F12}G_{L}C_{F}U_{F12}CC_{L}C_{F4}A_{L}C_{F4}C_{F} |
| | U_{F12}G_{L}C_{F4}U_{F}CC_{L4}C_{F4}A_{L}C_{F4}C_{F4} |
| | U_{F18}G_{L}C_{F4}U_{F}C_{F}C_{L4}C_{F}A_{L}C_{F4}C_{F} |
| | U_{F18}G_{L}C_{F}U_{F18}C_{F}C_{L}C_{F4}A_{L}C_{F4}C_{F} |
| | U_{F18}G_{L}C_{F4}U_{F}C_{F}C_{L4}C_{F4}A_{L}C_{F4}C_{F4} |
| | U_{F18}G_{L}C_{F4}U_{F}CC_{L4}C_{F}A_{L}C_{F4}C_{F} |
| | U_{F18}G_{L}C_{F}U_{F18}CC_{L}C_{F4}A_{L}C_{F4}C_{F} |
| | U_{F18}G_{L}C_{F4}U_{F}CC_{L4}C_{F4}A_{L}C_{F4}C_{F4} |
| | U_{L}G_{F}C_{L}U_{F}C_{F}C_{L}C_{F}A_{F}C_{L}C_{F} |
| | U_{L}G_{F}C_{L}U_{F}C_{F}C_{L}C_{F}A_{F}C_{L}C_{F} |
| | U_{L3}G_{F}C_{L4}U_{F}C_{F}C_{L4}C_{F}A_{F}C_{L4}C_{F} |
| | U_{L3}G_{F}C_{L}U_{F3}C_{F}C_{L}C_{F4}A_{F}C_{L4}C_{F} |
| | U_{L}G_{F}C_{L4}U_{F}C_{F}C_{L4}C_{F}A_{F}C_{L4}C_{F4} |
| | U_{L3}G_{F}C_{L4}U_{F}C_{F}C_{L4}C_{F4}A_{F}C_{L4}C_{F4} |
| | U_{L3}G_{F}C_{L4}U_{F}CC_{L4}C_{F}A_{F}C_{L4}C_{F} |
| | U_{L3}G_{F}C_{L}U_{F3}CC_{L}C_{F4}A_{F}C_{L4}C_{F} |
| | U_{L}G_{F}C_{L4}U_{F}CC_{L4}C_{F}A_{F}C_{L4}C_{F4} |
| | U_{L3}G_{F}C_{L4}U_{F}CC_{L4}C_{F4}A_{F}C_{L4}C_{F4} |
| | U_{L12}G_{F}C_{L4}U_{F}C_{F}C_{L4}C_{F}A_{F}C_{L4}C_{F} |
| | U_{L12}G_{F}C_{L}U_{F12}C_{F}C_{L}C_{F4}A_{F}C_{L4}C_{F} |
| | U_{L12}G_{F}C_{L4}U_{F}C_{F}C_{L4}C_{F4}A_{F}C_{L4}C_{F4} |
| | U_{L12}G_{F}C_{L4}U_{F}CC_{L4}C_{F}A_{F}C_{L4}C_{F} |
| | U_{L12}G_{F}C_{L}U_{F12}CC_{L}C_{F4}A_{F}C_{L4}C_{F} |
| | U_{L12}G_{F}C_{L4}U_{F}CC_{L4}C_{F4}A_{F}C_{L4}C_{F4} |
| | U_{L18}G_{F}C_{L4}U_{F}C_{F}C_{L4}C_{F}A_{F}C_{L4}C_{F} |
| | U_{L18}G_{F}C_{L}U_{F18}C_{F}C_{L}C_{F4}A_{F}C_{L4}C_{F} |
| | U_{L18}G_{F}C_{L4}U_{F}C_{F}C_{L4}C_{F4}A_{F}C_{L4}C_{F4} |
| | U_{L18}G_{F}C_{L4}U_{F}CC_{L4}C_{F}A_{F}C_{L4}C_{F} |
| | U_{L18}G_{F}C_{L}U_{F18}CC_{L}C_{F4}A_{F}C_{L4}C_{F} |
| | U_{L18}G_{F}C_{L4}U_{F}CC_{L4}C_{F4}A_{F}C_{L4}C_{F4} |
| | U_{F}G_{L}C_{F}U_{L}C_{F}C_{F}C_{F}A_{L}C_{F}C_{L} |
| | U_{F}G_{L}C_{F}U_{L}C_{F}C_{F}C_{F}A_{L}C_{F}C_{L} |
| | U_{F3}G_{L}C_{F4}U_{L}C_{F}C_{F4}C_{F}A_{L}C_{F4}C_{L} |
| | U_{F3}G_{L}C_{F}U_{L3}C_{F}C_{F}C_{F4}A_{L}C_{F4}C_{L} |
| | U_{F}G_{L}C_{F4}U_{L}C_{F}C_{F4}C_{F}A_{L}C_{F4}C_{L4} |
| | U_{F3}G_{L}C_{F4}U_{L}C_{F}C_{F4}C_{F4}A_{L}C_{F4}C_{L4} |
| | U_{F3}G_{L}C_{F4}U_{L}CC_{F4}C_{F}A_{L}C_{F4}C_{L} |
| | U_{F3}G_{L}C_{F}U_{L3}CC_{F}C_{F4}A_{L}C_{F4}C_{L} |
| | U_{F}G_{L}C_{F4}U_{L}CC_{F4}C_{F}A_{L}C_{F4}C_{L4} |
| | U_{F3}G_{L}C_{F4}U_{L}CC_{F4}C_{F4}A_{L}C_{F4}C_{L4} |
| | U_{F12}G_{L}C_{F4}U_{L}C_{F}C_{F4}C_{F}A_{L}C_{F4}C_{L} |
| | U_{F12}G_{L}C_{F}U_{L12}C_{F}C_{F}C_{F4}A_{L}C_{F4}C_{L} |
| | U_{F12}G_{L}C_{F4}U_{L}C_{F}C_{F4}C_{F4}A_{L}C_{F4}C_{L4} |
| | U_{F12}G_{L}C_{F4}U_{L}CC_{F4}C_{F}A_{L}C_{F4}C_{L} |
| | U_{F12}G_{L}C_{F}U_{L12}CC_{F}C_{F4}A_{L}C_{F4}C_{L} |
| | U_{F12}G_{L}C_{F4}U_{L}CC_{F4}C_{F4}A_{L}C_{F4}C_{L4} |
| | U_{F18}G_{L}C_{F4}U_{L}C_{F}C_{F4}C_{F}A_{L}C_{F4}C_{L} |
| | U_{F18}G_{L}C_{F}U_{L18}C_{F}C_{F}C_{F4}A_{L}C_{F4}C_{L} |
| | U_{F18}G_{L}C_{F4}U_{L}C_{F}C_{F4}C_{F4}A_{L}C_{F4}C_{L4} |
| | U_{F18}G_{L}C_{F4}U_{L}CC_{F4}C_{F}A_{L}C_{F4}C_{L} |
| | U_{F18}G_{L}C_{F}U_{L18}CC_{F}C_{F4}A_{L}C_{F4}C_{L} |
| | U_{F18}G_{L}C_{F4}U_{L}CC_{F4}C_{F4}A_{L}C_{F4}C_{L4} |

TABLE 19C
Examples of the Application of AGSD to the Seed
Vehicles used by Chorn et al. (2012) with a Higher
Level of Basic Nuclease Protection

| Seed | Compositions of Matter Suitable for Use |
|---|---|
| Vehicle | in vitro or in a Subject with or |
| Designation | without a Protective Carrier (5′-3′) |
| 7y_4 | G_{F}~U_{M}C_{F}~U_{M3}U_{F}~A_{M}U_{F}~U_{M3}~U_{F3}~G_{M} |
| | G_{F}~U_{M3}C_{F}~U_{M3}U_{F}~A_{M}U_{F3}~U_{M3}~U_{F3}~G_{M} |
| | G_{F}~U_{M3}C_{F}~U_{M3}U_{F3}~A_{M}U_{F3}~U_{M3}~U_{F}~G_{M} |
| | G_{F}~U_{M}C_{F4}~U_{M}U_{F}~A_{M}U_{F3}~U_{M3}~U_{F3}~G_{M} |
| | G_{F}~U_{M}C_{F}~U_{M3}U~A_{M}U_{F}~U_{M3}~U_{F3}~G_{M} |
| | G_{F}~U_{M3}C_{F}~U_{M3}U~A_{M}U_{F3}~U_{M3}~U_{F3}~G_{M} |
| | G_{F}~U_{M3}C_{F}~U_{M3}U₃~A_{M}U_{F3}~U_{M3}~U_{F}~G_{M} |
| | G_{F}~U_{M}C_{F4}~U_{M}U~A_{M}U_{F3}~U_{M3}~U_{F3}~G_{M} |
| | G_{F}~U_{M}C_{F}~U_{M12}U_{F}~A_{M}U_{F}~U_{M12}~U_{F12}~G_{M} |
| | G_{F}~U_{M12}C_{F}~U_{M12}U_{F}~A_{M}U_{F12}~U_{M12}~U_{F12}~G_{M} |
| | G_{F}~U_{M12}C_{F}~U_{M12}U_{F12}~A_{M}U_{F12}~U_{M12}~U_{F}~G_{M} |
| | G_{F}~U_{M}C_{F4}~U_{M}U_{F}~A_{M}U_{F12}~U_{M12}~U_{F12}~G_{M} |
| | G_{F}~U_{M}C_{F}~U_{M12}U~A_{M}U_{F}~U_{M12}~U_{F12}~G_{M} |
| | G_{F}~U_{M12}C_{F}~U_{M12}U~A_{M}U_{F12}~U_{M12}~U_{F12}~G_{M} |
| | G_{F}~U_{M12}C_{F}~U_{M12}U₁₂~A_{M}U_{F12}~U_{M12}~U_{F}~G_{M} |
| | G_{F}~U_{M}C_{F4}~U_{M}U~A_{M}U_{F12}~U_{M12}~U_{F12}~G_{M} |
| | G_{F}~U_{M}C_{F}~U_{M18}U_{F}~A_{M}U_{F}~U_{M18}~U_{F18}~G_{M} |
| | G_{F}~U_{M18}C_{F}~U_{M18}U_{F}~A_{M}U_{F18}~U_{M18}~U_{F18}~G_{M} |
| | G_{F}~U_{M18}C_{F}~U_{M18}U_{F18}~A_{M}U_{F18}~U_{M18}~U_{F}~G_{M} |
| | G_{F}~U_{M}C_{F4}~U_{M}U_{F}~A_{M}U_{F18}~U_{M18}~U_{F18}~G_{M} |
| | G_{F}~U_{M}C_{F}~U_{M18}U~A_{M}U_{F}~U_{M18}~U_{F18}~G_{M} |
| | G_{F}~U_{M18}C_{F}~U_{M18}U~A_{M}U_{F18}~U_{M18}~U_{F18}~G_{M} |
| | G_{F}~U_{M18}C_{F}~U_{M18}U₁₈~A_{M}U_{F18}~U_{M18}~U_{F}~G_{M} |
| | G_{F}~U_{M}C_{F4}~U_{M}U~A_{M}U_{F18}~U_{M18}~U_{F18}~G_{M} |
| | G_{F}~U_{L}C_{F}~U_{M}U_{F}~A_{L}U_{F}~U_{M}~U_{L}~G_{M} |
| | G_{F}~U_{L}C_{F}~U_{M}U_{F}~A_{L}U_{F}~U_{M}~U_{L}~G_{M} |
| | G_{F}~U_{L}C_{F}~U_{M3}U_{F}~A_{L}U_{F}~U_{M3}~U_{L3}~G_{M} |
| | G_{F}~U_{L3}C_{F}~U_{M3}U_{F}~A_{L}U_{F3}~U_{M3}~U_{L3}~G_{M} |
| | G_{F}~U_{L3}C_{F}~U_{M3}U_{F3}~A_{L}U_{F3}~U_{M3}~U_{L}~G_{M} |
| | G_{F}~U_{L}C_{F4}~U_{M}U_{F}~A_{L}U_{F3}~U_{M3}~U_{L3}~G_{M} |
| | G_{F}~U_{L}C_{F}~U_{M3}U~A_{L}U_{F}~U_{M3}~U_{L3}~G_{M} |
| | G_{F}~U_{L3}C_{F}~U_{M3}U~A_{L}U_{F3}~U_{M3}~U_{L3}~G_{M} |
| | G_{F}~U_{L3}C_{F}~U_{M3}U₃~A_{L}U_{F3}~U_{M3}~U_{L}~G_{M} |
| | G_{F}~U_{L}C_{F4}~U_{M}U~A_{L}U_{F3}~U_{M3}~U_{L3}~G_{M} |
| | G_{F}~U_{L}C_{F}~U_{M12}U_{F}~A_{L}U_{F}~U_{M12}~U_{L12}~G_{M} |
| | G_{F}~U_{L12}C_{F}~U_{M12}U_{F}~A_{L}U_{F12}~U_{M12}~U_{L12}~G_{M} |
| | G_{F}~U_{L12}C_{F}~U_{M12}U_{F12}~A_{L}U_{F12}~U_{M12}~U_{L}~G_{M} |
| | G_{F}~U_{L}C_{F4}~U_{M}U_{F}~A_{L}U_{F12}~U_{M12}~U_{L12}~G_{M} |
| | G_{F}~U_{L}C_{F}~U_{M12}U~A_{L}U_{F}~U_{M12}~U_{L12}~G_{M} |
| | G_{F}~U_{L12}C_{F}~U_{M12}U~A_{L}U_{F12}~U_{M12}~U_{L12}~G_{M} |
| | G_{F}~U_{L12}C_{F}~U_{M12}U₁₂~A_{L}U_{F12}~U_{M12}~U_{L}~G_{M} |
| | G_{F}~U_{L}C_{F4}~U_{M}U~A_{L}U_{F12}~U_{M12}~U_{L12}~G_{M} |
| | G_{F}~U_{L}C_{F}~U_{M18}U_{F}~A_{L}U_{F}~U_{M18}~U_{L18}~G_{M} |
| | G_{F}~U_{L18}C_{F}~U_{M18}U_{F}~A_{L}U_{F18}~U_{M18}~U_{L18}~G_{M} |
| | G_{F}~U_{L18}C_{F}~U_{M18}U_{F18}~A_{L}U_{F18}~U_{M18}~U_{L}~G_{M} |
| | G_{F}~U_{L}C_{F4}~U_{M}U_{F}~A_{L}U_{F18}~U_{M18}~U_{L18}~G_{M} |
| | G_{F}~U_{L}C_{F}~U_{M18}U~A_{L}U_{F}~U_{M18}~U_{L18}~G_{M} |
| | G_{F}~U_{L18}C_{F}~U_{M18}U~A_{L}U_{F18}~U_{M18}~U_{L18}~G_{M} |
| | G_{F}~U_{L18}C_{F}~U_{M18}U₁₈~A_{L}U_{F18}~U_{M18}~U_{L}~G_{M} |
| | G_{F}~U_{L}C_{F4}~U_{M}U~A_{L}U_{F18}~U_{M18}~U_{L18}~G_{M} |
| | G_{L}~U_{M}C_{F}~U_{L}U_{F}~A_{M}U_{F}~U_{L}~U_{F}~G_{M} |
| | G_{L}~U_{M}C_{F}~U_{L}U_{F}~A_{M}U_{F}~U_{L}~U_{F}~G_{M} |
| | G_{L}~U_{M}C_{F}~U_{L3}U_{F}~A_{M}U_{F}~U_{L3}~U_{F3}~G_{M} |
| | G_{L}~U_{M3}C_{F}~U_{L3}U_{F}~A_{M}U_{F3}~U_{L3}~U_{F3}~G_{M} |
| | G_{L}~U_{M3}C_{F}~U_{L3}U_{F3}~A_{M}U_{F3}~U_{L3}~U_{F}~G_{M} |
| | G_{L}~U_{M}C_{F4}~U_{L}U_{F}~A_{M}U_{F3}~U_{L3}~U_{F3}~G_{M} |
| | G_{L}~U_{M}C_{F}~U_{L3}U~A_{M}U_{F}~U_{L3}~U_{F3}~G_{M} |
| | G_{L}~U_{M3}C_{F}~U_{L3}U~A_{M}U_{F3}~U_{L3}~U_{F3}~G_{M} |
| | G_{L}~U_{M3}C_{F}~U_{L3}U₃~A_{M}U_{F3}~U_{L3}~U_{F}~G_{M} |
| | G_{L}~U_{M}C_{F4}~U_{L}U~A_{M}U_{F3}~U_{L3}~U_{F3}~G_{M} |
| | G_{L}~U_{M}C_{F}~U_{L12}U_{F}~A_{M}U_{F}~U_{L12}~U_{F12}~G_{M} |
| | G_{L}~U_{M12}C_{F}~U_{L12}U_{F}~A_{M}U_{F12}~U_{L12}~U_{F12}~G_{M} |
| | G_{L}~U_{M12}C_{F}~U_{L12}U_{F12}~A_{M}U_{F12}~U_{L12}~U_{F}~G_{M} |
| | G_{L}~U_{M}C_{F4}~U_{L}U_{F}~A_{M}U_{F12}~U_{L12}~U_{F12}~G_{M} |
| | G_{L}~U_{M}C_{F}~U_{L12}U~A_{M}U_{F}~U_{L12}~U_{F12}~G_{M} |
| | G_{L}~U_{M12}C_{F}~U_{L12}U~A_{M}U_{F12}~U_{L12}~U_{F12}~G_{M} |
| | G_{L}~U_{M12}C_{F}~U_{L12}U_{L12}~A_{M}U_{F12}~U_{L12}~U_{F}~G_{M} |
| | G_{L}~U_{M}C_{F4}~U_{L}U~A_{M}U_{F12}~U_{L12}~U_{F12}~G_{M} |
| | G_{L}~U_{M}C_{F}~U_{L18}U_{F}~A_{M}U_{F}~U_{L18}~U_{F18}~G_{M} |
| | G_{L}~U_{M18}C_{F}~U_{L18}U_{F}~A_{M}U_{F18}~U_{L18}~U_{F18}~G_{M} |
| | G_{L}~U_{M18}C_{F}~U_{L18}U_{L18}~A_{M}U_{F18}~U_{L18}~U_{F}~G_{M} |
| | G_{L}~U_{M}C_{F4}~U_{L}U_{F}~A_{M}U_{F18}~U_{L18}~U_{F18}~G_{M} |
| | G_{L}~U_{M}C_{F}~U_{L18}U~A_{M}U_{F}~U_{L18}~U_{F18}~G_{M} |
| | G_{L}~U_{M18}C_{F}~U_{L18}U~A_{M}U_{F18}~U_{L18}~U_{F18}~G_{M} |
| | G_{L}~U_{M18}C_{F}~U_{L18}U₁₈~A_{M}U_{F18}~U_{L18}~U_{F}~G_{M} |
| | G_{L}~U_{M}C_{F4}~U_{L}U~A_{M}U_{F18}~U_{L18}~U_{F18}~G_{M} |
| | G_{F}~U_{M}C_{L}~U_{M}U_{F}~A_{L}U_{F}~U_{M}~U_{L}~G_{M} |
| | G_{F}~U_{M}C_{L}~U_{M}U_{F}~A_{L}U_{F}~U_{M}~U_{L}~G_{M} |
| | G_{F}~U_{M}C_{L}~U_{M3}U_{F}~A_{L}U_{F}~U_{M3}~U_{L3}~G_{M} |
| | G_{F}~U_{M3}C_{L}~U_{M3}U_{F}~A_{L}U_{F3}~U_{M3}~U_{L3}~G_{M} |
| | G_{F}~U_{M3}C_{L}~U_{M3}U_{F3}~A_{L}U_{F3}~U_{M3}~U_{L}~G_{M} |
| | G_{F}~U_{M}C_{L4}~U_{M}U_{F}~A_{L}U_{F3}~U_{M3}~U_{L3}~G_{M} |
| | G_{F}~U_{M}C_{L}~U_{M3}U_{F}~A_{L}U_{F}~U_{M3}~U_{L3}~G_{M} |
| | G_{F}~U_{M3}C_{L}~U_{M3}U~A_{L}U_{F3}~U_{M3}~U_{L3}~G_{M} |
| | G_{F}~U_{M3}C_{L}~U_{M3}U₃~A_{L}U_{F3}~U_{M3}~U_{L}~G_{M} |
| | G_{F}~U_{M}C_{L4}~U_{M}U~A_{L}U_{F3}~U_{M3}~U_{L3}~G_{M} |
| | G_{F}~U_{M}C_{L}~U_{M12}U_{F}~A_{L}U_{F}~U_{M12}~U_{L12}~G_{M} |
| | G_{F}~U_{M12}C_{L}~U_{M12}U_{F}~A_{L}U_{F12}~U_{M12}~U_{L12}~G_{M} |
| | G_{F}~U_{M12}C_{L}~U_{M12}U_{F12}~A_{L}U_{F12}~U_{M12}~U_{L}~G_{M} |
| | G_{F}~U_{M}C_{L4}~U_{M}U_{F}~A_{L}U_{F12}~U_{M12}~U_{L12}~G_{M} |
| | G_{F}~U_{M}C_{L}~U_{M12}U~A_{L}U_{F}~U_{M12}~U_{L12}~G_{M} |
| | G_{F}~U_{M12}C_{L}~U_{M12}U~A_{L}U_{F12}~U_{M12}~U_{L12}~G_{M} |
| | G_{F}~U_{M12}C_{L}~U_{M12}U₁₂~A_{L}U_{F12}~U_{M12}~U_{L}~G_{M} |
| | G_{F}~U_{M}C_{L4}~U_{M}U~A_{L}U_{F12}~U_{M12}~U_{L12}~G_{M} |
| | G_{F}~U_{M}C_{L}~U_{M18}U_{F}~A_{L}U_{F}~U_{M18}~U_{L18}~G_{M} |
| | G_{F}~U_{M18}C_{L}~U_{M18}U_{F}~A_{L}U_{F18}~U_{M18}~U_{L18}~G_{M} |
| | G_{F}~U_{M18}C_{L}~U_{M18}U_{F18}~A_{L}U_{F18}~U_{M18}~U_{L}~G_{M} |
| | G_{F}~U_{M}C_{L4}~U_{M}U_{F}~A_{L}U_{F18}~U_{M18}~U_{L18}~G_{M} |
| | G_{F}~U_{M}C_{L}~U_{M18}U~A_{L}U_{F}~U_{M18}~U_{L18}~G_{M} |
| | G_{F}~U_{M18}C_{L}~U_{M18}U~A_{L}U_{F18}~U_{M18}~U_{L18}~G_{M} |
| | G_{F}~U_{M18}C_{L}~U_{M18}U₁₈~A_{L}U_{F18}~U_{M18}~U_{L}~G_{M} |
| | G_{F}~U_{M}C_{L4}~U_{M}U~A_{L}U_{F18}~U_{M18}~U_{L18}~G_{M} |
| | G_{F}~U_{L}C_{F}~U_{L}U_{F}~A_{M}U_{F}~U_{L}~U_{F}~G_{L} |
| | G_{F}~U_{L}C_{F}~U_{L}U_{F}~A_{M}U_{F}~U_{L}~U_{F}~G_{L} |
| | G_{F}~U_{L}C_{F}~U_{L3}U_{F}~A_{M}U_{F}~U_{L3}~U_{F3}~G_{M} |
| | G_{F}~U_{L3}C_{F}~U_{L3}U_{F}~A_{M}U_{F3}~U_{L3}~U_{F3}~G_{M} |
| | G_{F}~U_{L3}C_{F}~U_{L3}U_{F3}~A_{M}U_{F3}~U_{L3}~U_{F}~G_{M} |
| | G_{F}~U_{L}C_{F4}~U_{L}U_{F}~A_{M}U_{F3}~U_{L3}~U_{F3}~G_{M} |
| | G_{F}~U_{L}C_{F}~U_{L3}U~A_{M}U_{F}~U_{L3}~U_{F3}~G_{M} |
| | G_{F}~U_{L3}C_{F}~U_{L3}U~A_{M}U_{F3}~U_{L3}~U_{F3}~G_{M} |
| | G_{F}~U_{L3}C_{F}~U_{L3}U₃~A_{M}U_{F3}~U_{L3}~U_{F}~G_{M} |
| | G_{F}~U_{L}C_{F4}~U_{L}U~A_{M}U_{F3}~U_{L3}~U_{F3}~G_{M} |
| | G_{F}~U_{L}C_{F}~U_{L12}U_{F}~A_{M}U_{F}~U_{L12}~U_{F12}~G_{M} |
| | G_{F}~U_{L12}C_{F}~U_{L12}U_{F}~A_{M}U_{F12}~U_{L12}~U_{F12}~G_{M} |
| | G_{F}~U_{L12}C_{F}~U_{L12}U_{F12}~A_{M}U_{F12}~U_{L12}~U_{F}~G_{M} |
| | G_{F}~U_{L}C_{F4}~U_{L}U_{F}~A_{M}U_{F12}~U_{L12}~U_{F12}~G_{M} |
| | G_{F}~U_{L}C_{F}~U_{L12}U~A_{M}U_{F}~U_{L12}~U_{F12}~G_{M} |
| | G_{F}~U_{L12}C_{F}~U_{L12}U~A_{M}U_{F12}~U_{L12}~U_{F12}~G_{M} |
| | G_{F}~U_{L12}C_{F}~U_{L12}U₁₂~A_{M}U_{F12}~U_{L12}~U_{F}~G_{M} |
| | G_{F}~U_{L}C_{F4}~U_{L}U~A_{M}U_{F12}~U_{L12}~U_{F12}~G_{M} |
| | G_{F}~U_{L}C_{F}~U_{L18}U_{F}~A_{M}U_{F}~U_{L18}~U_{F18}~G_{M} |
| | G_{F}~U_{L18}C_{F}~U_{L18}U_{F}~A_{M}U_{F18}~U_{L18}~U_{F18}~G_{M} |
| | G_{F}~U_{L18}C_{F}~U_{L18}U_{F18}~A_{M}U_{F18}~U_{L18}~U_{F}~G_{M} |
| | G_{F}~U_{L}C_{F4}~U_{L}U_{F}~A_{M}U_{F18}~U_{L18}~U_{F18}~G_{M} |
| | G_{F}~U_{L}C_{F}~U_{L18}U~A_{M}U_{F}~U_{L18}~U_{F18}~G_{M} |
| | G_{F}~U_{L18}C_{F}~U_{L18}U_{F}~A_{M}U_{F18}~U_{L18}~U_{F18}~G_{M} |
| | G_{F}~U_{L18}C_{F}~U_{L18}U₁₈~A_{M}U_{F18}~U_{L18}~U_{F}~G_{M} |
| | G_{F}~U_{L}C_{F4}~U_{L}U~A_{M}U_{F18}~U_{L18}~U_{F18}~G_{M} |
| 8y_3 | U_{F3}~G_{M}C_{F4}~U_{M}C_{F}~C_{M4}C_{F}~A_{M}~C_{F4}~C_{M} |
| | U_{F3}~G_{M}C_{F}~U_{M3}C_{F}~C_{M}C_{F4}~A_{M}~C_{F4}~C_{M} |
| | U_{F}~G_{M}C_{F4}~U_{M}C_{F}~C_{M4}C_{F}~A_{M}~C_{F4}~C_{M4} |
| | U_{F3}~G_{M}C_{F4}~U_{M}C_{F4}~C_{M}C_{F4}~A_{M}~C_{F4}~C_{M4} |
| | U_{F3}~G_{M}C_{F4}~U_{M}C~C_{M4}C_{F}~A_{M}~C_{F4}~C_{M} |
| | U_{F3}~G_{M}C_{F}~U_{M3}C~C_{M}C_{F4}~A_{M}~C_{F4}~C_{M} |
| | U_{F}~G_{M}C_{F4}~U_{M}C~C_{M4}C_{F}~A_{M}~C_{F4}~C_{M4} |
| | U_{F3}~G_{M}C_{F4}~U_{M}C₄~C_{M}C_{F4}~A_{M}~C_{F4}~C_{M4} |
| | U_{F12}~G_{M}C_{F4}~U_{M}C_{F}~C_{M4}C_{F4}~A_{M}~C_{F4}~C_{M} |
| | U_{F12}~G_{M}C_{F}~U_{M12}C_{F}~C_{M}C_{F4}~A_{M}~C_{F4}~C_{M} |
| | U_{F12}~G_{M}C_{F4}~U_{M}C_{F4}~C_{M}C_{F4}~A_{M}~C_{F4}~C_{M4} |
| | U_{F12}~G_{M}C_{F4}~U_{M}C~C_{M4}C_{F}~A_{M}~C_{F4}~C_{M} |
| | U_{F12}~G_{M}C_{F}~U_{M12}C~C_{M}C_{F4}~A_{M}~C_{F4}~C_{M} |
| | U_{F12}~G_{M}C_{F4}~U_{M}C₄~A_{M}~C_{F4}~A_{M}~C_{F4}~C_{M4} |
| | U_{F18}~G_{M}C_{F4}~U_{M}C_{F}~C_{M4}C_{F}~A_{M}~C_{F4}~C_{M} |
| | U_{F18}~G_{M}C_{F}~U_{M18}C_{F}~C_{M}C_{F4}~A_{M}~C_{F4}~C_{M} |
| | U_{F18}~G_{M}C_{F4}~U_{M}C_{F4}~C_{M}C_{F4}~A_{M}~C_{F4}~C_{M4} |
| | U_{F18}~G_{M}C_{F4}~U_{M}C~C_{M4}C_{F}~A_{M}~C_{F4}~C_{M} |
| | U_{F18}~G_{M}C_{F}~U_{M18}C~C_{M}C_{F4}~A_{M}~C_{F4}~C_{M} |
| | U_{F18}~G_{M}C_{F4}~U_{M}C₄~C_{M}C_{F4}~A_{M}~C_{F4}~C_{M4} |
| | U_{F}~G_{L}C_{F}~U_{M}C_{F}~C_{L}C_{F}~A_{L}~C_{F}~C_{M} |
| | U_{F}~G_{L}C_{F}~U_{M}C~C_{L}C_{F}~A_{L}~C_{F}~C_{M} |
| | U_{F3}~G_{L}C_{F4}~U_{M}C_{F}~C_{L4}C_{F}~A_{L}~C_{F4}~C_{M} |
| | U_{F3}~G_{L}C_{F}~U_{M3}C_{F}~C_{L}C_{F4}~A_{L}~C_{F4}~C_{M} |
| | U_{F}~G_{L}C_{F4}~U_{M}C_{F}~C_{L4}C_{F}~A_{L}~C_{F4}~C_{M4} |
| | U_{F3}~G_{L}C_{F4}~U_{M}C_{F4}~C_{L}C_{F4}~A_{L}~C_{F4}~C_{M4} |
| | U_{F3}~G_{L}C_{F4}~U_{M}C~C_{L4}C_{F}~A_{L}~C_{F4}~C_{M} |
| | U_{F3}~G_{L}C_{F}~U_{M3}C~C_{L}C_{F4}~A_{L}~C_{F4}~C_{M} |
| | U_{F}~G_{L}C_{F4}~U_{M}C~C_{L4}C_{F}~A_{L}~C_{F4}~C_{M4} |
| | U_{F3}~G_{L}C_{F4}~U_{M}C₄~C_{L}C_{F4}~A_{L}~C_{F4}~C_{M4} |
| | U_{F12}~G_{L}C_{F4}~U_{M}C_{F}~C_{L4}C_{F}~A_{L}~C_{F4}~C_{M} |
| | U_{F12}~G_{L}C_{F}~U_{M12}C_{F}~C_{L}C_{F4}~A_{L}~C_{F4}~C_{M} |
| | U_{F12}~G_{L}C_{F4}~U_{M}C_{F4}~C_{L}C_{F4}~A_{L}~C_{F4}~C_{M4} |
| | U_{F12}~G_{L}C_{F4}~U_{M}C~C_{L4}C_{F}~A_{L}~C_{F4}~C_{M} |
| | U_{F12}~G_{L}C_{F}~U_{M12}C~C_{L}C_{F4}~A_{L}~C_{F4}~C_{M} |
| | U_{F12}~G_{L}C_{F4}~U_{M}C₄~C_{L}C_{F4}~A_{L}~C_{F4}~C_{M4} |
| | U_{F18}~G_{L}C_{F4}~U_{M}C_{F}~C_{L4}C_{F}~A_{L}~C_{F4}~C_{M} |
| | U_{F18}~G_{L}C_{F}~U_{M18}C_{F}~C_{L}C_{F4}~A_{L}~C_{F4}~C_{M} |
| | U_{F18}~G_{L}C_{F4}~U_{M}C_{F4}~C_{L}C_{F4}~A_{L}~C_{F4}~C_{M4} |
| | U_{F18}~G_{L}C_{F4}~U_{M}C~C_{L4}C_{F}~A_{L}~C_{F4}~C_{M} |
| | U_{F18}~G_{L}C_{F}~U_{M18}C~C_{L}C_{F4}~A_{L}~C_{F4}~C_{M} |
| | U_{F18}~G_{L}C_{F4}~U_{M}C₄~C_{L}C_{F4}~A_{L}~C_{F4}~C_{M4} |
| | U_{L}~G_{M}C_{L}~U_{M}C_{F}~C_{L}C_{F}~A_{M}~C_{L}~C_{M} |
| | U_{L}~G_{M}C_{L}~U_{M}C~C_{L}C_{F}~A_{M}~C_{L}~C_{M} |
| | U_{L3}~G_{M}C_{L4}~U_{M}C_{F}~C_{L4}C_{F}~A_{M}~C_{L4}~C_{M} |
| | U_{L3}~G_{M}C_{L}~U_{M3}C_{F}~C_{L}C_{F4}~A_{M}~C_{L4}~C_{M} |
| | U_{L}~G_{M}C_{L4}~U_{M}C_{F}~C_{L4}C_{F}~A_{M}~C_{L4}~C_{M4} |
| | U_{L3}~G_{M}C_{L4}~U_{M}C_{F4}~C_{L}C_{F4}~A_{M}~C_{L4}~C_{M4} |
| | U_{L3}~G_{M}C_{L4}~U_{M}C~C_{L4}C_{F}~A_{M}~C_{L4}~C_{M} |
| | U_{L3}~G_{M}C_{L}~U_{M3}C~C_{L}C_{F4}~A_{M}~C_{L4}~C_{M} |
| | U_{L}~G_{M}C_{L4}~U_{M}C~C_{L4}C_{F}~A_{M}~C_{L4}~C_{M4} |
| | _{L3}~G_{M}C_{L4}~U_{M}C₄~C_{L}C_{F4}~A_{M}~C_{L4}~C_{M4} |
| | U_{L12}~G_{M}C_{L4}~U_{M}C_{F}~C_{L4}C_{F}~A_{M}~C_{L4}~C_{M} |
| | U_{L12}~G_{M}C_{L}~U_{M12}C_{F}~C_{L}C_{F4}~A_{M}~C_{L4}~C_{M} |
| | U_{L12}~G_{M}C_{L4}~U_{M}C_{F4}~C_{L}C_{F4}~A_{M}~C_{L4}~C_{M4} |
| | U_{L12}~G_{M}C_{L4}~U_{M}C~C_{L4}C_{F}~A_{M}~C_{L4}~C_{M} |
| | U_{L12}~G_{M}C_{L}~U_{M12}C~C_{L}C_{F4}~A_{M}~C_{L4}~C_{M} |
| | U_{L12}~G_{M}C_{L4}~U_{M}C₄~C_{L}C_{F4}~A_{M}~C_{L4}~C_{M4} |
| | U_{L18}~G_{M}C_{L4}~U_{M}C_{F}~C_{L4}C_{F}~A_{M}~C_{L4}~C_{M} |
| | U_{L18}~G_{M}C_{L}~U_{M18}C_{F}~C_{L}C_{F4}~A_{M}~C_{L4}~C_{M} |
| | U_{L18}~G_{M}C_{L4}~U_{M}C_{F4}~C_{L}C_{F4}~A_{M}~C_{L4}~C_{M4} |
| | U_{L18}~G_{M}C_{L4}~U_{M}C~C_{L4}C_{F}~A_{M}~C_{L4}~C_{M} |
| | U_{L18}~G_{M}C_{L}~U_{M18}C~C_{L}C_{F4}~A_{M}~C_{L4}~C_{M} |
| | U_{L18}~G_{M}C_{L4}~U_{M}C₄~C_{L}C_{F4}~A_{M}~C_{L4}~C_{M4} |
| | U_{F}~G_{L}C_{F}~U_{L}C_{F}~C_{M}C_{F}~A_{L}~C_{F}~C_{L} |
| | U_{F}~G_{L}C_{F}~U_{L}C~C_{M}C_{F}~A_{L}~C_{F}~C_{L} |
| | U_{F3}~G_{L}C_{F4}~U_{L}C_{F}~C_{M4}C_{F}~A_{L}~C_{F4}~C_{L} |
| | U_{F3}~G_{L}C_{F}~U_{L3}C_{F}~C_{M}C_{F4}~A_{L}~C_{F4}~C_{L} |
| | U_{F}~G_{L}C_{F4}~U_{L}C_{F}~C_{M4}C_{F}~A_{L}~C_{F4}~C_{L4} |
| | U_{F3}~G_{L}C_{F4}~U_{L}C_{F4}~C_{M}C_{F4}~A_{L}~C_{F4}~C_{L4} |
| | U_{F3}~G_{L}C_{F4}~U_{L}C~C_{M4}C_{F}~A_{L}~C_{F4}~C_{L} |
| | U_{F3}~G_{L}C_{F}~U_{L3}C~C_{M}C_{F4}~A_{L}~C_{F4}~C_{L} |
| | U_{F}~G_{L}C_{F4}~U_{L}C~C_{M4}C_{F}~A_{L}~C_{F4}~C_{L4} |
| | U_{F3}~G_{L}C_{F4}~U_{L}C₄~C_{M}C_{F4}~A_{L}~C_{F4}~C_{L4} |
| | U_{F12}~G_{L}C_{F4}~U_{L}C_{F}~C_{M4}C_{F}~A_{L}~C_{F4}~C_{L} |
| | U_{F12}~G_{L}C_{F}~U_{L12}C_{F}~C_{M}C_{F4}~A_{L}~C_{F4}~C_{L} |
| | U_{F12}~G_{L}C_{F4}~U_{L}C_{F4}~C_{M}C_{F4}~A_{L}~C_{F4}~C_{L4} |
| | U_{F12}~G_{L}C_{F4}~U_{L}C~C_{M4}C_{F}~A_{L}~C_{F4}~C_{L} |
| | U_{F12}~G_{L}C_{F}~U_{L12}C~C_{M}C_{F4}~A_{L}~C_{F4}~C_{L} |
| | U_{F12}~G_{L}C_{F4}~U_{L}C₄~C_{M}C_{F4}~A_{L}~C_{F4}~C_{L4} |
| | U_{F18}~G_{L}C_{F4}~U_{L}C_{F}~C_{M4}C_{F}~A_{L}~C_{F4}~C_{L} |
| | U_{F18}~G_{L}C_{F}~U_{L18}C_{F}~C_{M}C_{F4}~A_{L}~C_{F4}~C_{L} |
| | U_{F18}~G_{L}C_{F4}~U_{L}C_{F4}~C_{M}C_{F4}~A_{L}~C_{F4}~C_{L4} |
| | U_{F18}~G_{L}C_{F4}~U_{L}C~C_{M4}C_{F}~A_{L}~C_{F4}~C_{L} |
| | U_{F18}~G_{L}C_{F}~U_{L18}C~C_{M}C_{F4}~A_{L}~C_{F4}~C_{L} |
| | U_{F18}~G_{L}C_{F4}~U_{L}C₄~C_{M}C_{F4}~A_{L}~C_{F4}~C_{L4} |
| 8y_4 | C_{F}~C_{M4}C_{F4}~U_{M}C_{F}~C_{M4}C_{F}~U_{M3}~G_{F}~A_{M} |
| | C_{F4}~C_{M4}C_{F}~U_{M3}C_{F}~C_{M}C_{F4}~U_{M3}~G_{F}~A_{M} |
| | C_{F4}~C_{M4}C_{F4}~U_{M}C_{F}~C_{M}C_{F}~U_{M3}~G_{F}~A_{M} |
| | C_{F4}~C_{M}C_{F4}~U_{M}C_{F}~C_{M4}C_{F4}~U_{M}~G_{F}~A_{M} |
| | C_{F}~C_{M4}C_{F4}~U_{M}C_{F}~C_{M4}C_{F}~U_{M3}~G_{F}~A_{M} |
| | C_{F4}~C_{M4}C_{F}~U_{M3}C_{F}~C_{M}C_{F4}~U_{M3}~G_{F}~A_{M} |
| | C_{F4}~C_{M4}C_{F4}~U_{M}C_{F}~C_{M}C_{F}~U_{M3}~G_{F}~A_{M} |
| | C_{F}~C_{M4}C_{F4}~U_{M}C~C_{M4}C_{F}~U_{M3}~G_{F}~A_{M} |
| | C_{F4}~C_{M4}C_{F}~U_{M3}C~C_{M}C_{F4}~U_{M3}~G_{F}~A_{M} |
| | C_{F4}~C_{M4}C_{F4}~U_{M}C~C_{M}C_{F}~U_{M3}~G_{F}~A_{M} |
| | C_{F4}~C_{M}C_{F4}~U_{M}C~C_{M4}C_{F4}~U_{M}~G_{F}~A_{M} |
| | C_{F}~C_{M4}C_{F4}~U_{M}C~C_{M4}C_{F}~U_{M3}~G_{F}~A_{M} |
| | C_{F4}~C_{M4}C_{F}~U_{M3}C~C_{M}C_{F4}~U_{M3}~G_{F}~A_{M} |
| | C_{F4}~C_{M4}C_{F4}~U_{M}C~C_{M}C_{F}~U_{M3}~G_{F}~A_{M} |
| | C_{F}~C_{M4}C_{F4}~U_{M}C_{F}~C_{M4}C_{F}~U_{M12}~G_{F}~A_{M} |
| | C_{F4}~C_{M4}C_{F}~U_{M12}C_{F}~C_{M}C_{F4}~U_{M12}~G_{F}~A_{M} |
| | C_{F4}~C_{M4}C_{F4}~U_{M}C_{F}~C_{M}C_{F}~U_{M12}~G_{F}~A_{M} |
| | C_{F}~C_{M4}C_{F4}~U_{M}C_{F}~C_{M4}C_{F}~U_{M12}~G_{F}~A_{M} |
| | C_{F4}~C_{M4}C_{F}~U_{M12}C_{F}~C_{M}C_{F4}~U_{M12}~G_{F}~A_{M} |
| | C_{F4}~C_{M4}C_{F4}~U_{M}C_{F}~C_{M}C_{F}~U_{M12}~G_{F}~A_{M} |
| | C_{F}~C_{M4}C_{F4}~U_{M}C~C_{M4}C_{F}~U_{M12}~G_{F}~A_{M} |
| | C_{F4}~C_{M4}C_{F}~U_{M12}C~C_{M}C_{F4}~U_{M12}~G_{F}~A_{M} |
| | C_{F4}~C_{M4}C_{F4}~U_{M}C~C_{M}C_{F}~U_{M12}~G_{F}~A_{M} |
| | C_{F}~C_{M4}C_{F4}~U_{M}C~C_{M4}C_{F}~U_{M12}~G_{F}~A_{M} |
| | C_{F4}~C_{M4}C_{F}~U_{M12}C~C_{M}C_{F4}~U_{M12}~G_{F}~A_{M} |
| | C_{F4}~C_{M4}C_{F4}~U_{M}C~C_{M}C_{F}~U_{M12}~G_{F}~A_{M} |
| | C_{F}~C_{M4}C_{F4}~U_{M}C_{F}~C_{M4}C_{F}~U_{M18}~G_{F}~A_{M} |
| | C_{F4}~C_{M4}C_{F}~U_{M18}C_{F}~C_{M}C_{F4}~U_{M18}~G_{F}~A_{M} |
| | C_{F4}~C_{M4}C_{F4}~U_{M}C_{F}~C_{M}C_{F}~U_{M18}~G_{F}~A_{M} |
| | C_{F}~C_{M4}C_{F4}~U_{M}C_{F}~C_{M4}C_{F}~U_{M18}~G_{F}~A_{M} |
| | C_{F4}~C_{M4}C_{F}~U_{M18}C_{F}~C_{M}C_{F4}~U_{M18}~G_{F}~A_{M} |
| | C_{F4}~C_{M4}C_{F4}~U_{M}C_{F}~C_{M}C_{F}~U_{M18}~G_{F}~A_{M} |
| | C_{F}~C_{M4}C_{F4}~U_{M}C~C_{M4}C_{F}~U_{M18}~G_{F}~A_{M} |
| | C_{F4}~C_{M4}C_{F}~U_{M18}C~C_{M}C_{F4}~U_{M18}~G_{F}~A_{M} |
| | C_{F4}~C_{M4}C_{F4}~U_{M}C~C_{M}C_{F}~U_{M18}~G_{F}~A_{M} |
| | C_{F}~C_{M4}C_{F4}~U_{M}C~C_{M4}C_{F}~U_{M18}~G_{F}~A_{M} |
| | C_{F4}~C_{M4}C_{F}~U_{M18}C~C_{M}C_{F4}~U_{M18}~G_{F}~A_{M} |
| | C_{F4}~C_{M4}C_{F4}~U_{M}C~C_{M}C_{F}~U_{M18}~G_{F}~A_{M} |
| | C_{F}~C_{L}C_{F}~U_{L}C_{F}~C_{L}C_{F}~U_{M}~G_{L}~A_{M} |
| | C_{F}~C_{L}C_{F}~U_{L}C~C_{L}C_{F}~U_{M}~G_{L}~A_{M} |
| | C_{F}~C_{L4}C_{F4}~U_{L}C_{F}~C_{L4}C_{F}~U_{M3}~G_{L}~A_{M} |
| | C_{F4}~C_{L4}C_{F}~U_{L3}C_{F}~C_{L}C_{F4}~U_{M3}~G_{L}~A_{M} |
| | C_{F4}~C_{L4}C_{F4}~U_{L}C_{F}~C_{L}C_{F}~U_{M3}~G_{L}~A_{M} |
| | C_{F4}~C_{L}C_{F4}~U_{L}C_{F}~C_{L4}C_{F4}~U_{M}~G_{L}~A_{M} |
| | C_{F}~C_{L4}C_{F4}~U_{L}C_{F}~C_{L4}C_{F}~U_{M3}~G_{L}~A_{M} |
| | C_{F4}~C_{L4}C_{F}~U_{L3}C_{F}~C_{L}C_{F4}~U_{M3}~G_{L}~A_{M} |
| | C_{F4}~C_{L4}C_{F4}~U_{L}C_{F}~C_{L}C_{F}~U_{M3}~G_{L}~A_{M} |
| | C_{F}~C_{L4}C_{F4}~U_{L}C~C_{L4}C_{F}~U_{M3}~G_{L}~A_{M} |
| | C_{F4}~C_{L4}C_{F}~U_{L3}C~C_{L}C_{F4}~U_{M3}~G_{L}~A_{M} |
| | C_{F4}~C_{L4}C_{F4}~U_{L}C~C_{L}C_{F}~U_{M3}~G_{L}~A_{M} |
| | C_{F4}~C_{L}C_{F4}~U_{L}C~C_{L4}C_{F4}~U_{M}~G_{L}~A_{M} |
| | C_{F}~C_{L4}C_{F4}~U_{L}C~C_{L4}C_{F}~U_{M3}~G_{L}~A_{M} |
| | C_{F4}~C_{L4}C_{F}~U_{L3}C~C_{L}C_{F4}~U_{M3}~G_{L}~A_{M} |
| | C_{F4}~C_{L4}C_{F4}~U_{L}C~C_{L}C_{F}~U_{M3}~G_{L}~A_{M} |
| | C_{F}~C_{L4}C_{F4}~U_{L}C_{F}~C_{L4}C_{F}~U_{M12}~G_{L}~A_{M} |
| | C_{F4}~C_{L4}C_{F}~U_{L12}C_{F}~C_{L}C_{F4}~U_{M12}~G_{L}~A_{M} |
| | C_{F4}~C_{L4}C_{F4}~U_{L}C_{F}~C_{L}C_{F}~U_{M12}~G_{L}~A_{M} |
| | C_{F}~C_{L4}C_{F4}~U_{L}C_{F}~C_{L4}C_{F}~U_{M12}~G_{L}~A_{M} |
| | C_{F4}~C_{L4}C_{F}~U_{L12}C_{F}~C_{L}C_{F4}~U_{M12}~G_{L}~A_{M} |
| | C_{F4}~C_{L4}C_{F4}~U_{L}C_{F}~C_{L}C_{F}~U_{M12}~G_{L}~A_{M} |
| | C_{F}~C_{L4}C_{F4}~U_{L}C~C_{L4}C_{F}~U_{M12}~G_{L}~A_{M} |
| | C_{F4}~C_{L4}C_{F}~U_{L12}C~C_{L}C_{F4}~U_{M12}~G_{L}~A_{M} |
| | C_{F4}~C_{L4}C_{F4}~U_{L}C~C_{L}C_{F}~U_{M12}~G_{L}~A_{M} |
| | C_{F}~C_{L4}C_{F4}~U_{L}C~C_{L4}C_{F}~U_{M12}~G_{L}~A_{M} |
| | C_{F4}~C_{L4}C_{F}~U_{L12}C~C_{L}C_{F4}~U_{M12}~G_{L}~A_{M} |
| | C_{F4}~C_{L4}C_{F4}~U_{L}C~C_{L}C_{F}~U_{M12}~G_{L}~A_{M} |
| | C_{F}~C_{L4}C_{F4}~U_{L}C_{F}~C_{L4}C_{F}~U_{M18}~G_{L}~A_{M} |
| | C_{F4}~C_{L4}C_{F}~U_{L18}C_{F}~C_{L}C_{F4}~U_{M18}~G_{L}~A_{M} |
| | C_{F4}~C_{L4}C_{F4}~U_{L}C_{F}~C_{L}C_{F}~U_{M18}~G_{L}~A_{M} |
| | C_{F}~C_{L4}C_{F4}~U_{L}C_{F}~C_{L4}C_{F}~U_{M18}~G_{L}~A_{M} |
| | C_{F4}~C_{L4}C_{F}~U_{L18}C_{F}~C_{L}C_{F4}~U_{M18}~G_{L}~A_{M} |
| | C_{F4}~C_{L4}C_{F4}~U_{L}C_{F}~C_{L}C_{F}~U_{M18}~G_{L}~A_{M} |
| | C_{F}~C_{L4}C_{F4}~U_{L}C~C_{L4}C_{F}~U_{M18}~G_{L}~A_{M} |
| | C_{F4}~C_{L4}C_{F}~U_{L18}C~C_{L}C_{F4}~U_{M18}~G_{L}~A_{M} |
| | C_{F4}~C_{L4}C_{F4}~U_{L}C~C_{L}C_{F}~U_{M18}~G_{L}~A_{M} |
| | C_{F}~C_{L4}C_{F4}~U_{L}C~C_{L4}C_{F}~U_{M18}~G_{L}~A_{M} |
| | C_{F4}~C_{L4}C_{F}~U_{L18}C~C_{L}C_{F4}~U_{M18}~G_{L}~A_{M} |
| | C_{F4}~C_{L4}C_{F4}~U_{L}C~C_{L}C_{F}~U_{M18}~G_{L}~A_{M} |
| | C_{L}~C_{M}C_{L}~U_{M}C_{F}~C_{M}C_{F}~U_{L}~G_{F}~A_{L} |
| | C_{L}~C_{M}C_{L}~U_{M}C~C_{M}C_{F}~U_{L}~G_{F}~A_{L} |
| | C_{L}~C_{M4}C_{L4}~U_{M}C_{F}~C_{M4}C_{F}~U_{L3}~G_{F}~A_{L} |
| | C_{L4}~C_{M4}C_{L}~U_{M3}C_{F}~C_{M}C_{F4}~U_{L3}~G_{F}~A_{L} |
| | C_{L4}~C_{M4}C_{L4}~U_{M}C_{F}~C_{M}C_{F}~U_{L3}~G_{F}~A_{L} |
| | C_{L4}~C_{M}C_{L4}~U_{M}C_{F}~C_{M4}C_{F4}~U_{L}~G_{F}~A_{L} |
| | C_{L}~C_{M4}C_{L4}~U_{M}C_{F}~C_{M4}C_{F}~U_{L3}~G_{F}~A_{L} |
| | C_{L4}~C_{M4}C_{L}~U_{M3}C_{F}~C_{M}C_{F4}~U_{L3}~G_{F}~A_{L} |
| | C_{L4}~C_{M4}C_{L4}~U_{M}C_{F}~C_{M}C_{F}~U_{L3}~G_{F}~A_{L} |
| | C_{L}~C_{M4}C_{L4}~U_{M}C~C_{M4}C_{F}~U_{L3}~G_{F}~A_{L} |
| | C_{L4}~C_{M4}C_{L}~U_{M3}C~C_{M}C_{F4}~U_{L3}~G_{F}~A_{L} |
| | C_{L4}~C_{M4}C_{L4}~U_{M}C~C_{M}C_{F}~U_{L3}~G_{F}~A_{L} |
| | C_{L4}~C_{M}C_{L4}~U_{M}C~C_{M4}C_{F4}~U_{L}~G_{F}~A_{L} |
| | C_{L}~C_{M4}C_{L4}~U_{M}C~C_{M4}C_{F}~U_{L3}~G_{F}~A_{L} |
| | C_{L4}~C_{M4}C_{L}~U_{M3}C~C_{M}C_{F4}~U_{L3}~G_{F}~A_{L} |
| | C_{L4}~C_{M4}C_{L4}~U_{M}C~C_{M}C_{F}~U_{L3}~G_{F}~A_{L} |
| | C_{L}~C_{M4}C_{L4}~U_{M}C_{F}~C_{M4}C_{F}~U_{L12}~G_{F}~A_{L} |
| | C_{L4}~C_{M4}C_{L}~U_{M12}C_{F}~C_{M}C_{F4}~U_{L12}~G_{F}~A_{L} |
| | C_{L4}~C_{M4}C_{L4}~U_{M}C_{F}~C_{M}C_{F}~U_{L12}~G_{F}~A_{L} |
| | C_{L}~C_{M4}C_{L4}~U_{M}C_{F}~C_{M4}C_{F}~U_{L12}~G_{F}~A_{L} |
| | C_{L4}~C_{M4}C_{L}~U_{M12}C_{F}~C_{M}C_{F4}~U_{L12}~G_{F}~A_{L} |
| | C_{L4}~C_{M4}C_{L4}~U_{M}C_{F}~C_{M}C_{F}~U_{L12}~G_{F}~A_{L} |
| | C_{L}~C_{M4}C_{L4}~U_{M}C~C_{M4}C_{F}~U_{L12}~G_{F}~A_{L} |
| | C_{L4}~C_{M4}C_{L}~U_{M12}C~C_{M}C_{F4}~U_{L12}~G_{F}~A_{L} |
| | C_{L4}~C_{M4}C_{L4}~U_{M}C~C_{M}C_{F}~U_{L12}~G_{F}~A_{L} |
| | C_{L}~C_{M4}C_{L4}~U_{M}C~C_{M4}C_{F}~U_{L12}~G_{F}~A_{L} |
| | C_{L4}~C_{M4}C_{L}~U_{M12}C~C_{M}C_{F4}~U_{L12}~G_{F}~A_{L} |
| | C_{L4}~C_{M4}C_{L4}~U_{M}C~C_{M}C_{F}~U_{L12}~G_{F}~A_{L} |
| | C_{L}~C_{M4}C_{L4}~U_{M}C_{F}~C_{M4}C_{F}~U_{L18}~G_{F}~A_{L} |
| | C_{L4}~C_{M4}C_{L}~U_{M18}C_{F}~C_{M}C_{F4}~U_{L18}~G_{F}~A_{L} |
| | C_{L4}~C_{M4}C_{L4}~U_{M}C_{F}~C_{M}C_{F}~U_{L18}~G_{F}~A_{L} |
| | C_{L}~C_{M4}C_{L4}~U_{M}C_{F}~C_{M4}C_{F}~U_{L18}~G_{F}~A_{L} |
| | C_{L4}~C_{M4}C_{L}~U_{M18}C_{F}~C_{M}C_{F4}~U_{L18}~G_{F}~A_{L} |
| | C_{L4}~C_{M4}C_{L4}~U_{M}C_{F}~C_{M}C_{F}~U_{L18}~G_{F}~A_{L} |
| | C_{L}~C_{M4}C_{L4}~U_{M}C~C_{M4}C_{F}~U_{L18}~G_{F}~A_{L} |
| | C_{L4}~C_{M4}C_{L}~U_{M18}C~C_{M}C_{F4}~U_{L18}~G_{F}~A_{L} |
| | C_{L4}~C_{M4}C_{L4}~U_{M}C~C_{M}C_{F}~U_{L18}~G_{F}~A_{L} |
| | C_{L}~C_{M4}C_{L4}~U_{M}C~C_{M4}C_{F}~U_{L18}~G_{F}~A_{L} |
| | C_{L4}~C_{M4}C_{L}~U_{M18}C~C_{M}C_{F4}~U_{L18}~G_{F}~A_{L} |
| | C_{L4}~C_{M4}C_{L4}~U_{M}C~C_{M}C_{F}~U_{L18}~G_{F}~A_{L} |
| | C_{F}~C_{M}C_{L}~U_{M}C_{F}~C_{L}C_{F}~U_{M}~G_{L}~A_{M} |
| | C_{F}~C_{M}C_{L}~U_{M}C~C_{L}C_{F}~U_{M}~G_{L}~A_{M} |
| | C_{F}~C_{M4}C_{L4}~U_{M}C_{F}~C_{L4}C_{F}~U_{M3}~G_{L}~A_{M} |
| | C_{F4}~C_{M4}C_{L}~U_{M3}C_{F}~C_{L}C_{F4}~U_{M3}~G_{L}~A_{M} |
| | C_{F4}~C_{M4}C_{L4}~U_{M}C_{F}~C_{L}C_{F}~U_{M3}~G_{L}~A_{M} |
| | C_{F4}~C_{M}C_{L4}~U_{M}C_{F}~C_{L4}C_{F4}~U_{M}~G_{L}~A_{M} |
| | C_{F}~C_{M4}C_{L4}~U_{M}C_{F}~C_{L4}C_{F}~U_{M3}~G_{L}~A_{M} |
| | C_{F4}~C_{M4}C_{L}~U_{M3}C_{F}~C_{L}C_{F4}~U_{M3}~G_{L}~A_{M} |
| | C_{F4}~C_{M4}C_{L4}~U_{M}C_{F}~C_{L}C_{F}~U_{M3}~G_{L}~A_{M} |
| | C_{F}~C_{M4}C_{L4}~U_{M}C~C_{L4}C_{F}~U_{M3}~G_{L}~A_{M} |
| | C_{F4}~C_{M4}C_{L}~U_{M3}C~C_{L}C_{F4}~U_{M3}~G_{L}~A_{M} |
| | C_{F4}~C_{M4}C_{L4}~U_{M}C~C_{L}C_{F}~U_{M3}~G_{L}~A_{M} |
| | C_{F4}~C_{M}C_{L4}~U_{M}C~C_{L4}C_{F4}~U_{M}~G_{L}~A_{M} |
| | C_{F}~C_{M4}C_{L4}~U_{M}C~C_{L4}C_{F}~U_{M3}~G_{L}~A_{M} |
| | C_{F4}~C_{M4}C_{L}~U_{M3}C~C_{L}C_{F4}~U_{M3}~G_{L}~A_{M} |
| | C_{F4}~C_{M4}C_{L4}~U_{M}C~C_{L}C_{F}~U_{M3}~G_{L}~A_{M} |
| | C_{F}~C_{M4}C_{L4}~U_{M}C_{F}~C_{L4}C_{F}~U_{M12}~G_{L}~A_{M} |
| | C_{F4}~C_{M4}C_{L}~U_{M12}C_{F}~C_{L}C_{F4}~U_{M12}~G_{L}~A_{M} |
| | C_{F4}~C_{M4}C_{L4}~U_{M}C_{F}~C_{L}C_{F}~U_{M12}~G_{L}~A_{M} |
| | C_{F}~C_{M4}C_{L4}~U_{M}C_{F}~C_{L4}C_{F}~U_{M12}~G_{L}~A_{M} |
| | C_{F4}~C_{M4}C_{L}~U_{M12}C_{F}~C_{L}C_{F4}~U_{M12}~G_{L}~A_{M} |
| | C_{F4}~C_{M4}C_{L4}~U_{M}C_{F}~C_{L}C_{F}~U_{M12}~G_{L}~A_{M} |
| | C_{F}~C_{M4}C_{L4}~U_{M}C~C_{L4}C_{F}~U_{M12}~G_{L}~A_{M} |
| | C_{F4}~C_{M4}C_{L}~U_{M12}C~C_{L}C_{F4}~U_{M12}~G_{L}~A_{M} |
| | C_{F4}~C_{M4}C_{L4}~U_{M}C~C_{L}C_{F}~U_{M12}~G_{L}~A_{M} |
| | C_{F}~C_{M4}C_{L4}~U_{M}C~C_{L4}C_{F}~U_{M12}~G_{L}~A_{M} |
| | C_{F4}~C_{M4}C_{L}~U_{M12}C~C_{L}C_{F4}~U_{M12}~G_{L}~A_{M} |
| | C_{F4}~C_{M4}C_{L4}~U_{M}C~C_{L}C_{F}~U_{M12}~G_{L}~A_{M} |
| | C_{F}~C_{M4}C_{L4}~U_{M}C_{F}~C_{L4}C_{F}~U_{M18}~G_{L}~A_{M} |
| | C_{F4}~C_{M4}C_{L}~U_{M18}C_{F}~C_{L}C_{F4}~U_{M18}~G_{L}~A_{M} |
| | C_{F4}~C_{M4}C_{L4}~U_{M}C_{F}~C_{L}C_{F}~U_{M18}~G_{L}~A_{M} |
| | C_{F}~C_{M4}C_{L4}~U_{M}C_{F}~C_{L4}C_{F}~U_{M18}~G_{L}~A_{M} |
| | C_{F4}~C_{M4}C_{L}~U_{M18}C_{F}~C_{L}C_{F4}~U_{M18}~G_{L}~A_{M} |
| | C_{F4}~C_{M4}C_{L4}~U_{M}C_{F}~C_{L}C_{F}~U_{M18}~G_{L}~A_{M} |
| | C_{F}~C_{M4}C_{L4}~U_{M}C~C_{L4}C_{F}~U_{M18}~G_{L}~A_{M} |
| | C_{F4}~C_{M4}C_{L}~U_{M18}C~C_{L}C_{F4}~U_{M18}~G_{L}~A_{M} |
| | C_{F4}~C_{M4}C_{L4}~U_{M}C~C_{L}C_{F}~U_{M18}~G_{L}~A_{M} |
| | C_{F}~C_{M4}C_{L4}~U_{M}C~C_{L4}C_{F}~U_{M18}~G_{L}~A_{M} |
| | C_{F4}~C_{M4}C_{L}~U_{M18}C~C_{L}C_{F4}~U_{M18}~G_{L}~A_{M} |
| | C_{F4}~C_{M4}C_{L4}~U_{M}C~C_{L}C_{F}~U_{M18}~G_{L}~A_{M} |
| 9y_2 | C_{F4}~G_{M}C_{F4}~U_{M}U_{F}~C_{M4}U_{F}~C_{M4}~U_{F}~U_{M} |
| | C_{F}~G_{M}C_{F}~U_{M3}U_{F}~C_{M4}U_{F}~C_{M}~U_{F3}~U_{M3} |
| | C_{F4}~G_{M}C_{F}~U_{M}U_{F}~C_{M}U_{F}~C_{M4}~U_{F}~U_{M3} |
| | C_{F}~G_{M}C_{F}~U_{M3}U_{F}~C_{M}U_{F3}~C_{M}~U_{F3}~U_{M} |
| | C_{F4}~G_{M}C_{F}~U_{M3}U_{F3}~C_{M}U_{F}~C_{M}~U_{F3}~U_{M3} |
| | C_{F4}~G_{M}C_{F4}~U_{M}U~C_{M4}U_{F}~C_{M4}~U_{F}~U_{M} |
| | C_{F}~G_{M}C_{F}~U_{M3}U~C_{M4}U_{F}~C_{M}~U_{F3}~U_{M3} |
| | C_{F4}~G_{M}C_{F}~U_{M}U~C_{M}U_{F}~C_{M4}~U_{F}~U_{M3} |
| | C_{F}~G_{M}C_{F}~U_{M3}U~C_{M}U_{F3}~C_{M}~U_{F3}~U_{M} |
| | C_{F4}~G_{M}C_{F}~U_{M3}U₃~C_{M}U_{F}~C_{M}~U_{F3}~U_{M3} |
| | C_{F}~G_{M}C_{F}~U_{M12}U_{F}~C_{M4}U_{F}~C_{M}~U_{F12}~U_{M12} |
| | C_{F4}~G_{M}C_{F}~U_{M}U_{F}~C_{M}U_{F}~C_{M4}~U_{F}~U_{M12} |
| | C_{F}~G_{M}C_{F}~U_{M12}U_{F}~C_{M}U_{F12}~C_{M}~U_{F12}~U_{M} |
| | C_{F4}~G_{M}C_{F}~U_{M12}U_{F12}~C_{M}U_{F}~C_{M}~U_{F12}~U_{M12} |
| | C_{F}~G_{M}C_{F}~U_{M12}U~C_{M4}U_{F}~C_{M}~U_{F12}~U_{M12} |
| | C_{F4}~G_{M}C_{F}~U_{M}U~C_{M}U_{F}~C_{M4}~U_{F}~U_{M12} |
| | C_{F}~G_{M}C_{F}~U_{M12}U~C_{M}U_{F12}~C_{M}~U_{F12}~U_{M} |
| | C_{F4}~G_{M}C_{F}~U_{M12}U₁₂~C_{M}U_{F}~C_{M}~U_{F12}~U_{M12} |
| | C_{F}~G_{M}C_{F}~U_{M18}U_{F}~C_{M4}U_{F}~C_{M}~U_{F18}~U_{M18} |
| | C_{F4}~G_{M}C_{F}~U_{M}U_{F}~C_{M}U_{F}~C_{M4}~U_{F}~U_{M18} |
| | C_{F}~G_{M}C_{F}~U_{M18}U_{F}~C_{M}U_{F18}~C_{M}~U_{F18}~U_{M} |
| | C_{F4}~G_{M}C_{F}~U_{M18}U_{F18}~C_{M}U_{F}~C_{M}~U_{F18}~U_{M18} |
| | C_{F}~G_{M}C_{F}~U_{M18}U~C_{M4}U_{F}~C_{M}~U_{F18}~U_{M18} |
| | C_{F4}~G_{M}C_{F}~U_{M}U~C_{M}U_{F}~C_{M4}~U_{F}~U_{M18} |
| | C_{F}~G_{M}C_{F}~U_{M18}U~C_{M}U_{F18}~C_{M}~U_{F18}~U_{M} |
| | C_{F4}~G_{M}C_{F}~U_{M18}U₁₈~C_{M}U_{F}~C_{M}~U_{F18}~U_{M18} |
| | C_{F}~G_{L}C_{F}~U_{M}U_{F}~C_{L}U_{F}~C_{M}~U_{L}~U_{M} |
| | C_{F}~G_{L}C_{F}~U_{M}U~C_{L}U_{F}~C_{M}~U_{L}~U_{M} |
| | C_{F4}~G_{L}C_{F4}~U_{M}U_{F}~C_{L4}U_{F}~C_{M4}~U_{L}~U_{M} |
| | C_{F}~G_{L}C_{F}~U_{M3}U_{F}~C_{L4}U_{F}~C_{M}~U_{L3}~U_{M3} |
| | C_{F4}~G_{L}C_{F}~U_{M}U_{F}~C_{L}U_{F}~C_{M4}~U_{L}~U_{M3} |
| | C_{F}~G_{L}C_{F}~U_{M3}U_{F}~C_{L}U_{F3}~C_{M}~U_{L3}~U_{M} |
| | C_{F4}~G_{L}C_{F}~U_{M3}U_{F3}~C_{L}U_{F}~C_{M}~U_{L3}~U_{M3} |
| | C_{F4}~G_{L}C_{F4}~U_{M}U~C_{L4}U_{F}~C_{M4}~U_{L}~U_{M} |
| | C_{F}~G_{L}C_{F}~U_{M3}U~C_{L4}U_{F}~C_{M}~U_{L3}~U_{M3} |
| | C_{F4}~G_{L}C_{F}~U_{M}U~C_{L}U_{F}~C_{M4}~U_{L}~U_{M3} |
| | C_{F}~G_{L}C_{F}~U_{M3}U~C_{L}U_{F3}~C_{M}~U_{L3}~U_{M} |
| | C_{F4}~G_{L}C_{F}~U_{M3}U₃~C_{L}U_{F}~C_{M}~U_{L3}~U_{M3} |
| | C_{F}~G_{L}C_{F}~U_{M12}U_{F}~C_{L4}U_{F}~C_{M}~U_{L12}~U_{M12} |
| | C_{F4}~G_{L}C_{F}~U_{M}U_{F}~C_{L}U_{F}~C_{M4}~U_{L}~U_{M12} |
| | C_{F}~G_{L}C_{F}~U_{M12}U_{F}~C_{L}U_{F12}~C_{M}~U_{L12}~U_{M} |
| | C_{F4}~G_{L}C_{F}~U_{M12}U_{F12}~C_{L}U_{F}~C_{M}~U_{L12}~U_{M12} |
| | C_{F}~G_{L}C_{F}~U_{M12}U~C_{L4}U_{F}~C_{M}~U_{L12}~U_{M12} |
| | C_{F4}~G_{L}C_{F}~U_{M}U~C_{L}U_{F}~C_{M4}~U_{L}~U_{M12} |
| | C_{F}~G_{L}C_{F}~U_{M12}U~C_{L}U_{F12}~C_{M}~U_{L12}~U_{M} |
| | C_{F4}~G_{L}C_{F}~U_{M12}U₁₂~C_{L}U_{F}~C_{M}~U_{L12}~U_{M12} |
| | C_{F}~G_{L}C_{F}~U_{M18}U_{F}~C_{L4}U_{F}~C_{M}~U_{L18}~U_{M18} |
| | C_{F4}~G_{L}C_{F}~U_{M}U_{F}~C_{L}U_{F}~C_{M4}~U_{L}~U_{M18} |
| | C_{F}~G_{L}C_{F}~U_{M18}U_{F}~C_{L}U_{F18}~C_{M}~U_{L18}~U_{M} |
| | C_{F4}~G_{L}C_{F}~U_{M18}U_{F18}~C_{L}U_{F}~C_{M}~U_{L18}~U_{M18} |
| | C_{F}~G_{L}C_{F}~U_{M18}U~C_{L4}U_{F}~C_{M}~U_{L18}~U_{M18} |
| | C_{F4}~G_{L}C_{F}~U_{M}U~C_{L}U_{F}~C_{M4}~U_{L}~U_{M18} |
| | C_{F}~G_{L}C_{F}~U_{M18}U~C_{L}U_{F18}~C_{M}~U_{L18}~U_{M} |
| | C_{F4}~G_{L}C_{F}~U_{M18}U₁₈~C_{L}U_{F}~C_{M}~U_{L18}~U_{M18} |
| | C_{L}~G_{M}C_{L}~U_{M}U_{F}~C_{M}U_{F}~C_{L}~U_{F}~U_{M} |
| | C_{L}~G_{M}C_{L}~U_{M}U~C_{M}U_{F}~C_{L}~U_{F}~U_{M} |
| | C_{L4}~G_{M}C_{L4}~U_{M}U_{F}~C_{M4}U_{F}~C_{L4}~U_{F}~U_{M} |
| | C_{L}~G_{M}C_{L}~U_{M3}U_{F}~C_{M4}U_{F}~C_{L}~U_{F3}~U_{M3} |
| | C_{L4}~G_{M}C_{L}~U_{M}U_{F}~C_{M}U_{F}~C_{L4}~U_{F}~U_{M3} |
| | C_{L}~G_{M}C_{L}~U_{M3}U_{F}~C_{M}U_{F3}~C_{L}~U_{F3}~U_{M} |
| | C_{L4}~G_{M}C_{L}~U_{M3}U_{F3}~C_{M}U_{F}~C_{L}~U_{F3}~U_{M3} |
| | C_{L4}~G_{M}C_{L4}~U_{M}U~C_{M4}U_{F}~C_{L4}~U_{F}~U_{M} |
| | C_{L}~G_{M}C_{L}~U_{M3}U~C_{M4}U_{F}~C_{L}~U_{F3}~U_{M3} |
| | C_{L4}~G_{M}C_{L}~U_{M}U~C_{M}U_{F}~C_{L4}~U_{F}~U_{M3} |
| | C_{L}~G_{M}C_{L}~U_{M3}U~C_{M}U_{F3}~C_{L}~U_{F3}~U_{M} |
| | C_{F4}~G_{M}C_{L}~U_{M3}U₃~C_{M}U_{F}~C_{L}~U_{F3}~U_{M3} |
| | C_{L}~G_{M}C_{L}~U_{M12}U_{F}~C_{M4}U_{F}~C_{L}~U_{F12}~U_{M12} |
| | C_{L4}~G_{M}C_{L}~U_{M}U_{F}~C_{M}U_{F}~C_{L4}~U_{F}~U_{M12} |
| | C_{L}~G_{M}C_{L}~U_{M12}U_{F}~C_{M}U_{F12}~C_{L}~U_{F12}~U_{M} |
| | C_{L4}~G_{M}C_{L}~U_{M12}U_{F12}~C_{M}U_{F}~C_{L}~U_{F12}~U_{M12} |
| | C_{L}~G_{M}C_{L}~U_{M12}U~C_{M4}U_{F}~C_{L}~U_{F12}~U_{M12} |
| | C_{L4}~G_{M}C_{L}~U_{M}U~C_{M}U_{F}~C_{L4}~U_{F}~U_{M12} |
| | C_{L}~G_{M}C_{L}~U_{M12}U~C_{M}U_{F12}~C_{L}~U_{F12}~U_{M} |
| | C_{L4}~G_{M}C_{L}~U_{M12}U₁₂~C_{M}U_{F}~C_{L}~U_{F12}~U_{M12} |
| | C_{L}~G_{M}C_{L}~U_{M18}U_{F}~C_{M4}U_{F}~C_{L}~U_{F18}~U_{M18} |
| | C_{L4}~G_{M}C_{L}~U_{M}U_{F}~C_{M}U_{F}~C_{L4}~U_{F}~U_{M18} |
| | C_{L}~G_{M}C_{L}~U_{M18}U_{F}~C_{M}U_{F18}~C_{L}~U_{F18}~U_{M} |
| | C_{L4}~G_{M}C_{L}~U_{M18}U_{F18}~C_{M}U_{F}~C_{L}~U_{F18}~U_{M18} |
| | C_{L}~G_{M}C_{L}~U_{M18}U~C_{M4}U_{F}~C_{L}~U_{F18}~U_{M18} |
| | C_{L4}~G_{M}C_{L}~U_{M}U~C_{M}U_{F}~C_{L4}~U_{F}~U_{M18} |
| | C_{L}~G_{M}C_{L}~U_{M18}U~C_{M}U_{F18}~C_{L}~U_{F18}~U_{M} |
| | C_{L4}~G_{M}C_{L}~U_{M18}U₁₈~C_{M}U_{F}~C_{L}~U_{F18}~U_{M18} |
| | C_{F}~G_{L}C_{F}~U_{L}U_{F}~C_{L}U_{F}~C_{L}~U_{F}~U_{L} |
| | C_{F}~G_{L}C_{F}~U_{L}U~C_{L}U_{F}~C_{L}~U_{F}~U_{L} |
| | C_{F4}~G_{L}C_{F4}~U_{L}U_{F}~C_{L4}U_{F}~C_{L4}~U_{F}~U_{L} |
| | C_{F}~G_{L}C_{F}~U_{L3}U_{F}~C_{L4}U_{F}~C_{L}~U_{F3}~U_{L3} |
| | C_{F4}~G_{L}C_{F}~U_{L}U_{F}~C_{L}U_{F}~C_{L4}~U_{F}~U_{L3} |
| | C_{F}~G_{L}C_{F}~U_{L3}U_{F}~C_{L}U_{F3}~C_{L}~U_{F3}~U_{L} |
| | C_{F4}~G_{L}C_{F}~U_{L3}U_{F3}~C_{L}U_{F}~C_{L}~U_{F3}~U_{L3} |
| | C_{F4}~G_{L}C_{F4}~U_{L}U~C_{L4}U_{F}~C_{L4}~U_{F}~U_{L} |
| | C_{F}~G_{L}C_{F}~U_{L3}U~C_{L4}U_{F}~C_{L}~U_{F3}~U_{L3} |
| | C_{F4}~G_{L}C_{F}~U_{L}U~C_{L}U_{F}~C_{L4}~U_{F}~U_{L3} |
| | C_{F}~G_{L}C_{F}~U_{L3}U~C_{L}U_{F3}~C_{L}~U_{F3}~U_{L} |
| | C_{F4}~G_{L}C_{F}~U_{L3}U₃~C_{L}U_{F}~C_{L}~U_{F3}~U_{L3} |
| | C_{F}~G_{L}C_{F}~U_{L12}U_{F}~C_{L4}U_{F}~C_{L}~U_{F12}~U_{L12} |
| | C_{F4}~G_{L}C_{F}~U_{L}U_{F}~C_{L}U_{F}~C_{L4}~U_{F}~U_{L12} |
| | C_{F}~G_{L}C_{F}~U_{L12}U_{F}~C_{L}U_{F12}~C_{L}~U_{F12}~U_{L} |
| | C_{F4}~G_{L}C_{F}~U_{L12}U_{F12}~C_{L}U_{F}~C_{L}~U_{F12}~U_{L12} |
| | C_{F}~G_{L}C_{F}~U_{L12}U~C_{L4}U_{F}~C_{L}~U_{F12}~U_{L12} |
| | C_{F4}~G_{L}C_{F}~U_{L}U~C_{L}U_{F}~C_{L4}~U_{F}~U_{L12} |
| | C_{F}~G_{L}C_{F}~U_{L12}U~C_{L}U_{F12}~C_{L}~U_{F12}~U_{L} |
| | C_{F4}~G_{L}C_{F}~U_{L12}U₁₂~C_{L}U_{F}~C_{L}~U_{F12}~U_{L12} |
| | C_{F}~G_{L}C_{F}~U_{L18}U_{F}~C_{L4}U_{F}~C_{L}~U_{F18}~U_{L18} |
| | C_{F4}~G_{L}C_{F}~U_{L}U_{F}~C_{L}U_{F}~C_{L4}~U_{F}~U_{L18} |
| | C_{F}~G_{L}C_{F}~U_{L18}U_{F}~C_{L}U_{F18}~C_{L}~U_{F18}~U_{L} |
| | C_{F4}~G_{L}C_{F}~U_{L18}U_{F18}~C_{L}U_{F}~C_{L}~U_{F18}~U_{L18} |
| | C_{F}~G_{L}C_{F}~U_{L18}U~C_{L4}U_{F}~C_{L}~U_{F18}~U_{L18} |
| | C_{F4}~G_{L}C_{F}~U_{L}U~C_{L}U_{F}~C_{L4}~U_{F}~U_{L18} |
| | C_{F}~G_{L}C_{F}~U_{L18}U~C_{L}U_{F18}~C_{L}~U_{F18}~U_{L} |
| | C_{F4}~G_{L}C_{F}~U_{L18}U₁₈~C_{L}U_{F}~C_{L}~U_{F18}~U_{L18} |
| 9y_3 | U_{F3}~U_{M}C_{F4}~U_{M}C_{F}~G_{M}U_{F}~C_{M4}~U_{F}~C_{M4} |
| | U_{F3}~U_{M3}C_{F}~U_{M3}C_{F}~G_{M}U_{F3}~C_{M}~U_{F3}~C_{M} |
| | U_{F}~U_{M}C_{F4}~U_{M}C_{F4}~G_{M}U_{F}~C_{M4}~U_{F}~C_{M4} |
| | U_{F3}~U_{M3}C_{F}~U_{M3}C_{F4}~G_{M}U_{F3}~C_{M4}~U_{F3}~C_{M} |
| | U_{F12}~U_{M}C_{F4}~U_{M}C_{F}~G_{M}U_{F}~C_{M4}~U_{F}~C_{M4} |
| | U_{F12}~U_{M12}C_{F}~U_{M12}C_{F}~G_{M}U_{F12}~C_{M}~U_{F12}~C_{M} |
| | U_{F12}~U_{M12}C_{F}~U_{M12}C_{F4}~G_{M}U_{F12}~C_{M4}~U_{F12}~C_{M} |
| | U_{F18}~U_{M}C_{F4}~U_{M}C_{F}~G_{M}U_{F}~C_{M4}~U_{F}~C_{M4} |
| | U_{F18}~U_{M18}C_{F}~U_{M18}C_{F}~G_{M}U_{F18}~C_{M}~U_{F18}~C_{M} |
| | U_{F18}~U_{M18}C_{F}~U_{M18}C_{F4}~G_{M}U_{F18}~C_{M4}~U_{F18}~C_{M} |
| | U_{F3}~U_{M}C_{F4}~U_{M}C~G_{M}U_{F}~C_{M4}~U_{F}~C_{M4} |
| | U_{F3}~U_{M3}C_{F}~U_{M3}C~G_{M}U_{F3}~C_{M}~U_{F3}~C_{M} |
| | U_{F}~U_{M}C_{F4}~U_{M}C₄~G_{M}U_{F}~C_{M4}~U_{F}~C_{M4} |
| | U_{F3}~U_{M3}C_{F}~U_{M3}C₄~G_{M}U_{F3}~C_{M4}~U_{F3}~C_{M} |
| | U_{F12}~U_{M}C_{F4}~U_{M}C~G_{M}U_{F}~C_{M4}~U_{F}~C_{M4} |
| | U_{F12}~U_{M12}C_{F}~U_{M12}C~G_{M}U_{F12}~C_{M}~U_{F12}~C_{M} |
| | U_{F12}~U_{M12}C_{F}~U_{M12}C₄~G_{M}U_{F12}~C_{M4}~U_{F12}~C_{M} |
| | U_{F18}~U_{M}C_{F4}~U_{M}C~G_{M}U_{F}~C_{M4}~U_{F}~C_{M4} |
| | U_{F18}~U_{M18}C_{F}~U_{M18}C~G_{M}U_{F18}~C_{M}~U_{F18}~C_{M} |
| | U_{F18}~U_{M18}C_{F}~U_{M18}C₄~G_{M}U_{F18}~C_{M4}~U_{F18}~C_{M} |
| | U_{F}~U_{L}C_{F}~U_{M}C_{F}~G_{L}U_{F}~C_{M}~U_{L}~C_{M} |
| | U_{F}~U_{L}C_{F}~U_{M}C~G_{L}U_{F}~C_{M}~U_{L}~C_{M} |
| | U_{F3}~U_{L}C_{F4}~U_{M}C_{F}~G_{L}U_{F}~C_{M4}~U_{L}~C_{M4} |
| | U_{F3}~U_{L3}C_{F}~U_{M3}C_{F}~G_{L}U_{F3}~C_{M}~U_{L3}~C_{M} |
| | U_{F}~U_{L}C_{F4}~U_{M}C_{F4}~G_{L}U_{F}~C_{M4}~U_{L}~C_{M4} |
| | U_{F3}~U_{L3}C_{F}~U_{M3}C_{F4}~G_{L}U_{F3}~C_{M4}~U_{L3}~C_{M} |
| | U_{F12}~U_{L}C_{F4}~U_{M}C_{F}~G_{L}U_{F}~C_{M4}~U_{L}~C_{M4} |
| | U_{F12}~U_{L12}C_{F}~U_{M12}C_{F}~G_{L}U_{F12}~C_{M}~U_{L12}~C_{M} |
| | U_{F12}~U_{L12}C_{F}~U_{M12}C_{F4}~G_{L}U_{F12}~C_{M4}~U_{L12}~C_{M} |
| | U_{F18}~U_{L}C_{F4}~U_{M}C_{F}~G_{L}U_{F}~C_{M4}~U_{L}~C_{M4} |
| | U_{F18}~U_{L18}C_{F}~U_{M18}C_{F}~G_{L}U_{F18}~C_{M}~U_{L18}~C_{M} |
| | U_{F18}~U_{L18}C_{F}~U_{M18}C_{F4}~G_{L}U_{F18}~C_{M4}~U_{L18}~C_{M} |
| | U_{F3}~U_{L}C_{F4}~U_{M}C~G_{L}U_{F}~C_{M4}~U_{L}~C_{M4} |
| | U_{F3}~U_{L3}C_{F}~U_{M3}C~G_{L}U_{F3}~C_{M}~U_{L3}~C_{M} |
| | U_{F}~U_{L}C_{F4}~U_{M}C₄~G_{L}U_{F}~C_{M4}~U_{L}~C_{M4} |
| | U_{F3}~U_{L3}C_{F}~U_{M3}C₄~G_{L}U_{F3}~C_{M4}~U_{L3}~C_{M} |
| | U_{F12}~U_{L}C_{F4}~U_{M}C~G_{L}U_{F}~C_{M4}~U_{L}~C_{M4} |
| | U_{F12}~U_{L12}C_{F}~U_{M12}C~G_{L}U_{F12}~C_{M}~U_{L12}~C_{M} |
| | U_{F12}~U_{L12}C_{F}~U_{M12}C₄~G_{L}U_{F12}~C_{M4}~U_{L12}~C_{M} |
| | U_{F18}~U_{L}C_{F4}~U_{M}C~G_{L}U_{F}~C_{M4}~U_{L}~C_{M4} |
| | U_{F18}~U_{L18}C_{F}~U_{M18}C~G_{L}U_{F18}~C_{M}~U_{L18}~C_{M} |
| | U_{F18}~U_{L18}C_{F}~U_{M18}C₄~G_{L}U_{F18}~C_{M4}~U_{L18}~C_{M} |
| | U_{F}~U_{M}C_{L}~U_{M}C_{F}~G_{L}U_{F}~C_{L}~U_{F}~C_{L} |
| | U_{F}~U_{M}C_{L}~U_{M}C~G_{L}U_{F}~C_{L}~U_{F}~C_{L} |
| | U_{F3}~U_{M}C_{L4}~U_{M}C_{F}~G_{L}U_{F}~C_{L4}~U_{F}~C_{L4} |
| | U_{F3}~U_{M3}C_{L}~U_{M3}C_{F}~G_{L}U_{F3}~C_{L}~U_{F3}~C_{L} |
| | U_{F}~U_{M}C_{L4}~U_{M}C_{F4}~G_{L}U_{F}~C_{L4}~U_{F}~C_{L4} |
| | U_{F3}~U_{M3}C_{L}~U_{M3}C_{F4}~G_{L}U_{F3}~C_{L4}~U_{F3}~C_{L} |
| | U_{F12}~U_{M}C_{L4}~U_{M}C_{F}~G_{L}U_{F}~C_{L4}~U_{F}~C_{L4} |
| | U_{F12}~U_{M12}C_{L}~U_{M12}C_{F}~G_{L}U_{F12}~C_{L}~U_{F12}~C_{L} |
| | U_{F12}~U_{M12}C_{L}~U_{M12}C_{F4}~G_{L}U_{F12}~C_{L4}~U_{F12}~C_{L} |
| | U_{F18}~U_{M}C_{L4}~U_{M}C_{F}~G_{L}U_{F}~C_{L4}~U_{F}~C_{L4} |
| | U_{F18}~U_{M18}C_{L}~U_{M18}C_{F}~G_{L}U_{F18}~C_{L}~U_{F18}~C_{L} |
| | U_{F18}~U_{M18}C_{L}~U_{M18}C_{F4}~G_{L}U_{F18}~C_{L4}~U_{F18}~C_{L} |
| | U_{F3}~U_{M}C_{L4}~U_{M}C~G_{L}U_{F}~C_{L4}~U_{F}~C_{L4} |
| | U_{F3}~U_{M3}C_{L}~U_{M3}C~G_{L}U_{F3}~C_{L}~U_{F3}~C_{L} |
| | U_{F}~U_{M}C_{L4}~U_{M}C₄~G_{L}U_{F}~C_{L4}~U_{F}~C_{L4} |
| | U_{F3}~U_{M3}C_{L}~U_{M3}C₄~G_{L}U_{F3}~C_{L4}~U_{F3}~C_{L} |
| | U_{F12}~U_{M}C_{L4}~U_{M}C~G_{L}U_{F}~C_{L4}~U_{F}~C_{L4} |
| | U_{F12}~U_{M12}C_{L}~U_{M12}C~G_{L}U_{F12}~C_{L}~U_{F12}~C_{L} |
| | U_{F12}~U_{M12}C_{L}~U_{M12}C₄~G_{L}U_{F12}~C_{L4}~U_{F12}~C_{L} |
| | U_{F18}~U_{M}C_{L4}~U_{M}C~G_{L}U_{F}~C_{L4}~U_{F}~C_{L4} |
| | U_{F18}~U_{M18}C_{L}~U_{M18}C~G_{L}U_{F18}~C_{L}~U_{F18}~C_{L} |
| | U_{F18}~U_{M18}C_{L}~U_{M18}C₄~G_{L}U_{F18}~C_{L4}~U_{F18}~C_{L} |
| | U_{L}~U_{M}C_{F}~U_{L}C_{F}~G_{M}U_{L}~C_{M}~U_{L}~C_{M} |
| | U_{L}~U_{M}C_{F}~U_{L}C~G_{M}U_{L}~C_{M}~U_{L}~C_{M} |
| | U_{L3}~U_{M}C_{F4}~U_{L}C_{F}~G_{M}U_{L}~C_{M4}~U_{L}~C_{M4} |
| | U_{L3}~U_{M3}C_{F}~U_{L3}C_{F}~G_{M}U_{L3}~C_{M}~U_{L3}~C_{M} |
| | U_{L}~U_{M}C_{F4}~U_{L}C_{F4}~G_{M}U_{L}~C_{M4}~U_{L}~C_{M4} |
| | U_{L3}~U_{M3}C_{F}~U_{L3}C_{F4}~G_{M}U_{L3}~C_{M4}~U_{L3}~C_{M} |
| | U_{L12}~U_{M}C_{F4}~U_{L}C_{F}~G_{M}U_{L}~C_{M4}~U_{L}~C_{M4} |
| | U_{L12}~U_{M12}C_{F}~U_{L12}C_{F}~G_{M}U_{L12}~C_{M}~U_{L12}~C_{M} |
| | U_{L12}~U_{M12}C_{F}~U_{L12}C_{F4}~G_{M}U_{L12}~C_{M4}~U_{L12}~C_{M} |
| | U_{L18}~U_{M}C_{F4}~U_{L}C_{F}~G_{M}U_{L}~C_{M4}~U_{L}~C_{M4} |
| | U_{L18}~U_{M18}C_{F}~U_{L18}C_{F}~G_{M}U_{L18}~C_{M}~U_{L18}~C_{M} |
| | U_{L18}~U_{M18}C_{F}~U_{L18}C_{F4}~G_{M}U_{L18}~C_{M4}~U_{L18}~C_{M} |
| | U_{L3}~U_{M}C_{F4}~U_{L}C~G_{M}U_{L}~C_{M4}~U_{L}~C_{M4} |
| | U_{L3}~U_{M3}C_{F}~U_{L3}C~G_{M}U_{L3}~C_{M}~U_{L3}~C_{M} |
| | U_{L}~U_{M}C_{F4}~U_{L}C₄~G_{M}U_{L}~C_{M4}~U_{L}~C_{M4} |
| | U_{L3}~U_{M3}C_{F}~U_{L3}C₄~G_{M}U_{L3}~C_{M4}~U_{L3}~C_{M} |
| | U_{L12}~U_{M}C_{F4}~U_{L}C~G_{M}U_{L}~C_{M4}~U_{L}~C_{M4} |
| | U_{L12}~U_{M12}C_{F}~U_{L12}C~G_{M}U_{L12}~C_{M}~U_{L12}~C_{M} |
| | U_{L12}~U_{M12}C_{F}~U_{L12}C₄~G_{M}U_{L12}~C_{M4}~U_{L12}~C_{M} |
| | U_{L18}~U_{M}C_{F4}~U_{L}C~G_{M}U_{L}~C_{M4}~U_{L}~C_{M4} |
| | U_{L18}~U_{M18}C_{F}~U_{L18}C~G_{M}U_{L18}~C_{M}~U_{L18}~C_{M} |
| | U_{L18}~U_{M18}C_{F}~U_{L18}C₄~G_{M}U_{L18}~C_{M4}~U_{L18}~C_{M} |
| 10y_1 | U_{F3}~C_{M}C_{F4}~C_{M}U_{F}~U_{M3}C_{F}~U_{M}~U_{F3}~C_{M} |
| | U_{F}~C_{M4}C_{F}~C_{M4}U_{F}~U_{M}C_{F}~U_{M3}~U_{F}~C_{M4} |
| | U_{F}~C_{M4}C_{F4}~C_{M}U_{F}~U_{M3}C_{F}~U_{M3}~U_{F3}~C_{M} |
| | U_{F}~C_{M4}C_{F4}~C_{M4}U_{F}~U_{M}C_{F4}~U_{M}~U_{F3}~C_{M} |
| | U_{F12}~C_{M}C_{F4}~C_{M}U_{F}~U_{M12}C_{F}~U_{M}~U_{F12}~C_{M} |
| | U_{F}~C_{M4}C_{F}~C_{M4}U_{F}~U_{M}C_{F}~U_{M12}~U_{F}~C_{M4} |
| | U_{F}~C_{M4}C_{F4}~C_{M}U_{F}~U_{M12}C_{F}~U_{M12}~U_{F12}~C_{M} |
| | U_{F}~C_{M4}C_{F4}~C_{M4}U_{F}~U_{M}C_{F4}~U_{M}~U_{F12}~C_{M} |
| | U_{F18}~C_{M}C_{F4}~C_{M}U_{F}~U_{M18}C_{F}~U_{M}~U_{F18}~C_{M} |
| | U_{F}~C_{M4}C_{F}~C_{M4}U_{F}~U_{M}C_{F}~U_{M18}~U_{F}~C_{M4} |
| | U_{F}~C_{M4}C_{F4}~C_{M}U_{F}~U_{M18}C_{F}~U_{M18}~U_{F18}~C_{M} |
| | U_{F}~C_{M4}C_{F4}~C_{M4}U_{F}~U_{M}C_{F4}~U_{M}~U_{F18}~C_{M} |
| | U_{F3}~C_{M}C_{F4}~C_{M}U~U_{M3}C_{F}~U_{M}~U_{F3}~C_{M} |
| | U_{F}~C_{M4}C_{F}~C_{M4}U~U_{M}C_{F}~U_{M3}~U_{F}~C_{M4} |
| | U_{F}~C_{M4}C_{F4}~C_{M}U~U_{M3}C_{F}~U_{M3}~U_{F3}~C_{M} |
| | U_{F}~C_{M4}C_{F4}~C_{M4}U~U_{M}C_{F4}~U_{M}~U_{F3}~C_{M} |
| | U_{F12}~C_{M}C_{F4}~C_{M}U~U_{M12}C_{F}~U_{M}~U_{F12}~C_{M} |
| | U_{F}~C_{M4}C_{F}~C_{M4}U~U_{M}C_{F}~U_{M12}~U_{F}~C_{M4} |
| | U_{F}~C_{M4}C_{F4}~C_{M}U~U_{M12}C_{F}~U_{M12}~U_{F12}~C_{M} |
| | U_{F}~C_{M4}C_{F4}~C_{M4}U~U_{M}C_{F4}~U_{M}~U_{F12}~C_{M} |
| | U_{F18}~C_{M}C_{F4}~C_{M}U~U_{M18}C_{F}~U_{M}~U_{F18}~C_{M} |
| | U_{F}~C_{M4}C_{F}~C_{M4}U~U_{M}C_{F}~U_{M18}~U_{F}~C_{M4} |
| | U_{F}~C_{M4}C_{F4}~C_{M}U~U_{M18}C_{F}~U_{M18}~U_{F18}~C_{M} |
| | U_{F}~C_{M4}C_{F4}~C_{M4}U~U_{M}C_{F4}~U_{M}~U_{F18}~C_{M} |
| | U_{F}~C_{L}C_{F}~C_{M}U_{F}~U_{L}C_{F}~U_{M}~U_{L}~C_{M} |
| | U_{F}~C_{L}C_{F}~C_{M}U~U_{L}C_{F}~U_{M}~U_{L}~C_{M} |
| | U_{F3}~C_{L}C_{F4}~C_{M}U_{F}~U_{L3}C_{F}~U_{M}~U_{L3}~C_{M} |
| | U_{F}~C_{L4}C_{F}~C_{M4}U_{F}~U_{L}C_{F}~U_{M3}~U_{L}~C_{M4} |
| | U_{F}~C_{L4}C_{F4}~C_{M}U_{F}~U_{L3}C_{F}~U_{M3}~U_{L3}~C_{M} |
| | U_{F}~C_{L4}C_{F4}~C_{M4}U_{F}~U_{L}C_{F4}~U_{M}~U_{L3}~C_{M} |
| | U_{F12}~C_{L}C_{F4}~C_{M}U_{F}~U_{L12}C_{F}~U_{M}~U_{L12}~C_{M} |
| | U_{F}~C_{L4}C_{F}~C_{M4}U_{F}~U_{L}C_{F}~U_{M12}~U_{L}~C_{M4} |
| | U_{F}~C_{L4}C_{F4}~C_{M}U_{F}~U_{L12}C_{F}~U_{M12}~U_{L12}~C_{M} |
| | U_{F}~C_{L4}C_{F4}~C_{M4}U_{F}~U_{L}C_{F4}~U_{M}~U_{L12}~C_{M} |
| | U_{F18}~C_{L}C_{F4}~C_{M}U_{F}~U_{L18}C_{F}~U_{M}~U_{L18}~C_{M} |
| | U_{F}~C_{L4}C_{F}~C_{M4}U_{F}~U_{L}C_{F}~U_{M18}~U_{L}~C_{M4} |
| | U_{F}~C_{L4}C_{F4}~C_{M}U_{F}~U_{L18}C_{F}~U_{M18}~U_{L18}~C_{M} |
| | U_{F}~C_{L4}C_{F4}~C_{M4}U_{F}~U_{L}C_{F4}~U_{M}~U_{L18}~C_{M} |
| | U_{F3}~C_{L}C_{F4}~C_{M}U~U_{L3}C_{F}~U_{M}~U_{L3}~C_{M} |
| | U_{F}~C_{L4}C_{F}~C_{M4}U~U_{L}C_{F}~U_{M3}~U_{L}~C_{M4} |
| | U_{F}~C_{L4}C_{F4}~C_{M}U~U_{L3}C_{F}~U_{M3}~U_{L3}~C_{M} |
| | U_{F}~C_{L4}C_{F4}~C_{M4}U~U_{L}C_{F4}~U_{M}~U_{L3}~C_{M} |
| | U_{F12}~C_{L}~C_{F4}~C_{M}U~U_{L12}C_{F}~U_{M}~U_{L12}~C_{M} |
| | U_{F}~C_{L4}C_{F}~C_{M4}U~U_{L}C_{F}~U_{M12}~U_{L}~C_{M4} |
| | U_{F}~C_{L4}C_{F4}~C_{M}U~U_{L12}C_{F}~U_{M12}~U_{L12}~C_{M} |
| | U_{F}~C_{L4}C_{F4}~C_{M4}U~U_{L}C_{F4}~U_{M}~U_{L12}~C_{M} |
| | U_{F18}~C_{L}~C_{F4}~C_{M}U~U_{L18}C_{F}~U_{M}~U_{L18}~C_{M} |
| | U_{F}~C_{L4}C_{F}~C_{M4}U~U_{L}C_{F}~U_{M18}~U_{L}~C_{M4} |
| | U_{F}~C_{L4}C_{F4}~C_{M}U~U_{L18}C_{F}~U_{M18}~U_{L18}~C_{M} |
| | U_{F}~C_{L4}C_{F4}~C_{M4}U~U_{L}C_{F4}~U_{M}~U_{L18}~C_{M} |
| | U_{F}~C_{L}~C_{F}~C_{L}~U_{F}~U_{M}C_{F}~U_{M}~U_{L}~C_{M} |
| | U_{F}~C_{L}~C_{F}~C_{L}~U~U_{M}C_{F}~U_{M}~U_{L}~C_{M} |
| | U_{F3}~C_{L}~C_{F4}~C_{L}~U_{F}~U_{M3}C_{F}~U_{M}~U_{L3}~C_{M} |
| | U_{F}~C_{L4}C_{F}~C_{L4}U_{F}~U_{M}C_{F}~U_{M3}~U_{L}~C_{M4} |
| | U_{F}~C_{L4}C_{F4}~C_{L}~U_{F}~U_{M3}C_{F}~U_{M3}~U_{L3}~C_{M} |
| | U_{F}~C_{L4}C_{F4}~C_{L4}U_{F}~U_{M}C_{F4}~U_{M}~U_{L3}~C_{M} |
| | U_{F12}~C_{L}~C_{F4}~C_{L}~U_{F}~U_{M12}C_{F}~U_{M}~U_{L12}~C_{M} |
| | U_{F}~C_{L4}C_{F}~C_{L4}U_{F}~U_{M}C_{F}~U_{M12}~U_{L}~C_{M4} |
| | U_{F}~C_{L4}C_{F4}~C_{L}~U_{F}~U_{M12}C_{F}~U_{M12}~U_{L12}~C_{M} |
| | U_{F}~C_{L4}C_{F4}~C_{L4}U_{F}~U_{M}C_{F4}~U_{M}~U_{L12}~C_{M} |
| | U_{F18}~C_{L}~C_{F4}~C_{L}~U_{F}~U_{M18}C_{F}~U_{M}~U_{L18}~C_{M} |
| | U_{F}~C_{L4}C_{F}~C_{L4}U_{F}~U_{M}C_{F}~U_{M18}~U_{L}~C_{M4} |
| | U_{F}~C_{L4}C_{F4}~C_{L}~U_{F}~U_{M18}C_{F}~U_{M18}~U_{L18}~C_{M} |
| | U_{F}~C_{L4}C_{F4}~C_{L4}U_{F}~U_{M}C_{F4}~U_{M}~U_{L18}~C_{M} |
| | U_{F3}~C_{L}~C_{F4}~C_{L}~U~U_{M3}C_{F}~U_{M}~U_{L3}~C_{M} |
| | U_{F}~C_{L4}C_{F}~C_{L4}U~U_{M}C_{F}~U_{M3}~U_{L}~C_{M4} |
| | U_{F}~C_{L4}C_{F4}~C_{L}~U~U_{M3}C_{F}~U_{M3}~U_{L3}~C_{M} |
| | U_{F}~C_{L4}C_{F4}~C_{L4}U~U_{M}C_{F4}~U_{M}~U_{L3}~C_{M} |
| | U_{F12}~C_{L}~C_{F4}~C_{L}~U~U_{M12}C_{F}~U_{M}~U_{L12}~C_{M} |
| | U_{F}~C_{L4}C_{F}~C_{L4}U~U_{M}C_{F}~U_{M12}~U_{L}~C_{M4} |
| | U_{F}~C_{L4}C_{F4}~C_{L}~U~U_{M12}C_{F}~U_{M12}~U_{L12}~C_{M} |
| | U_{F}~C_{L4}C_{F4}~C_{L4}U~U_{M}C_{F4}~U_{M}~U_{L12}~C_{M} |
| | U_{F18}~C_{L}~C_{F4}~C_{L}~U~U_{M18}C_{F}~U_{M}~U_{L18}~C_{M} |
| | U_{F}~C_{L4}C_{F}~C_{L4}U~U_{M}C_{F}~U_{M18}~U_{L}~C_{M4} |
| | U_{F}~C_{L4}C_{F4}~C_{L}~U~U_{M18}C_{F}~U_{M18}~U_{L18}~C_{M} |
| | U_{F}~C_{L4}C_{F4}~C_{L4}U~U_{M}C_{F4}~U_{M}~U_{L18}~C_{M} |
| | U_{L}~C_{M}C_{L}~C_{M}U_{F}~U_{M}C_{F}~U_{L}~U_{F}~C_{L} |
| | U_{L}~C_{M}C_{L}~C_{M}U~U_{M}C_{F}~U_{L}~U_{F}~C_{L} |
| | U_{L3}~C_{M}C_{L4}~C_{M}U_{F}~U_{M3}C_{F}~U_{L}~U_{F3}~C_{L} |
| | U_{L}~C_{M4}C_{L}~C_{M4}U_{F}~U_{M}C_{F}~U_{L3}~U_{F}~C_{L4} |
| | U_{L}~C_{M4}C_{L4}~C_{M}U_{F}~U_{M3}C_{F}~U_{L3}~U_{F3}~C_{L} |
| | U_{L}~C_{M4}C_{L4}~C_{M4}U_{F}~U_{M}C_{F4}~U_{L}~U_{F3}~C_{L} |
| | U_{L12}~C_{M}C_{L4}~C_{M}U_{F}~U_{M12}C_{F}~U_{L}~U_{F12}~C_{L} |
| | U_{L}~C_{M4}C_{L}~C_{M4}U_{F}~U_{M}C_{F}~U_{L12}~U_{F}~C_{L4} |
| | U_{L}~C_{M4}C_{L4}~C_{M}U_{F}~U_{M12}C_{F}~U_{L12}~U_{F12}~C_{L} |
| | U_{L}~C_{M4}C_{L4}~C_{M4}U_{F}~U_{M}C_{F4}~U_{L}~U_{F12}~C_{L} |
| | U_{L18}~C_{M}C_{L4}~C_{M}U_{F}~U_{M18}C_{F}~U_{L}~U_{F18}~C_{L} |
| | U_{L}~C_{M4}C_{L}~C_{M4}U_{F}~U_{M}C_{F}~U_{L18}~U_{F}~C_{L4} |
| | U_{L}~C_{M4}C_{L4}~C_{M}U_{F}~U_{M18}C_{F}~U_{L18}~U_{F18}~C_{L} |
| | U_{L}~C_{M4}C_{L4}~C_{M4}U_{F}~U_{M}C_{F4}~U_{L}~U_{F18}~C_{L} |
| | U_{L3}~C_{M}C_{L4}~C_{M}U~U_{M3}C_{F}~U_{L}~U_{F3}~C_{L} |
| | U_{L}~C_{M4}C_{L}~C_{M4}U~U_{M}C_{F}~U_{L3}~U_{F}~C_{L4} |
| | U_{L}~C_{M4}C_{L4}~C_{M}U~U_{M3}C_{F}~U_{L3}~U_{F3}~C_{L} |
| | U_{L}~C_{M4}C_{L4}~C_{M4}U~U_{M}C_{F4}~U_{L}~U_{F3}~C_{L} |
| | U_{L12}~C_{M}C_{L4}~C_{M}U~U_{M12}C_{F}~U_{L}~U_{F12}~C_{L} |
| | U_{L}~C_{M4}C_{L}~C_{M4}U~U_{M}C_{F}~U_{L12}~U_{F}~C_{L4} |
| | U_{L}~C_{M4}C_{L4}~C_{M}U~U_{M12}C_{F}~U_{L12}~U_{F12}~C_{L} |
| | U_{L}~C_{M4}C_{L4}~C_{M4}U~U_{M}C_{F4}~U_{L}~U_{F12}~C_{L} |
| | U_{L18}~C_{M}C_{L4}~C_{M}U~U_{M18}C_{F}~U_{L}~U_{F18}~C_{L} |
| | U_{L}~C_{M4}C_{L}~C_{M4}U~U_{M}C_{F}~U_{L18}~U_{F}~C_{L4} |
| | U_{L}~C_{M4}C_{L4}~C_{M}U~U_{M18}C_{F}~U_{L18}~U_{F18}~C_{L} |
| | U_{L}~C_{M4}C_{L4}~C_{M4}U~U_{M}C_{F4}~U_{L}~U_{F18}~C_{L} |
| 10y_7 | C_{F4}~U_{M}C_{F4}~C_{M}C_{F}~U_{M}U_{F}~U_{M}~C_{F4}~U_{M} |
| | C_{F}~U_{M3}C_{F}~C_{M}C_{F}~U_{M3}U_{F}~U_{M3}~C_{F}~U_{M3} |
| | C_{F4}~U_{M}C_{F4}~C_{M}C_{F}~U_{M}U_{F3}~U_{M}~C_{F4}~U_{M} |
| | C_{F}~U_{M}C_{F4}~C_{M4}C_{F}~U_{M3}U_{F3}~U_{M3}~C_{F}~U_{M3} |
| | C_{F}~U_{M}C_{F4}~C_{M4}C_{F4}~U_{M3}U_{F3}~U_{M3}~C_{F}~U_{M3} |
| | C_{F}~U_{M12}C_{F}~C_{M}C_{F}~U_{M12}U_{F}~U_{M12}~C_{F}~U_{M12} |
| | C_{F4}~U_{M}C_{F4}~C_{M}C_{F}~U_{M}U_{F12}~U_{M}~C_{F4}~U_{M} |
| | C_{F}~U_{M}C_{F4}~C_{M4}C_{F}~U_{M12}U_{F12}~U_{M12}~C_{F}~U_{M12} |
| | C_{F}~U_{M}C_{F4}~C_{M4}C_{F4}~U_{M12}U_{F12}~U_{M12}~C_{F}~U_{M12} |
| | C_{F}~U_{M18}C_{F}~C_{M}C_{F}~U_{M18}U_{F}~U_{M18}~C_{F}~U_{M18} |
| | C_{F4}~U_{M}C_{F4}~C_{M}C_{F}~U_{M}U_{F18}~U_{M}~C_{F4}~U_{M} |
| | C_{F}~U_{M}C_{F4}~C_{M4}C_{F}~U_{M18}U_{F18}~U_{M18}~C_{F}~U_{M18} |
| | C_{F}~U_{M}C_{F4}~C_{M4}C_{F4}~U_{M18}U_{F18}~U_{M18}~C_{F}~U_{M18} |
| | C_{F4}~U_{M}C_{F4}~C_{M}C~U_{M}U_{F}~U_{M}~C_{F4}~U_{M} |
| | C_{F}~U_{M3}C_{F}~C_{M}C~U_{M3}U_{F}~U_{M3}~C_{F}~U_{M3} |
| | C_{F4}~U_{M}C_{F4}~C_{M}C~U_{M}U_{F3}~U_{M}~C_{F4}~U_{M} |
| | C_{F}~U_{M}C_{F4}~C_{M4}C~U_{M3}U_{F3}~U_{M3}~C_{F}~U_{M3} |
| | C_{F}~U_{M}C_{F4}~C_{M4}C₄~U_{M3}U_{F3}~U_{M3}~C_{F}~U_{M3} |
| | C_{F}~U_{M12}C_{F}~C_{M}C~U_{M12}U_{F}~U_{M12}~C_{F}~U_{M12} |
| | C_{F4}~U_{M}C_{F4}~C_{M}C~U_{M}U_{F12}~U_{M}~C_{F4}~U_{M} |
| | C_{F}~U_{M}C_{F4}~C_{M4}C~U_{M12}U_{F12}~U_{M12}~C_{F}~U_{M12} |
| | C_{F}~U_{M}C_{F4}~C_{M4}C₄~U_{M12}U_{F12}~U_{M12}~C_{F}~U_{M12} |
| | C_{F}~U_{M18}C_{F}~C_{M}C~U_{M18}U_{F}~U_{M18}~C_{F}~U_{M18} |
| | C_{F4}~U_{M}C_{F4}~C_{M}C~U_{M}U_{F18}~U_{M}~C_{F4}~U_{M} |
| | C_{F}~U_{M}C_{F4}~C_{M4}C~U_{M18}U_{F18}~U_{M18}~C_{F}~U_{M18} |
| | C_{F}~U_{M}C_{F4}~C_{M4}C₄~U_{M18}U_{F18}~U_{M18}~C_{F}~U_{M18} |
| | C_{F}~U_{L}C_{F}~C_{M}C_{F}~U_{L}U_{F}~U_{M}~C_{L}~U_{M} |
| | C_{F}~U_{L}C_{F}~C_{M}C~U_{L}U_{F}~U_{M}~C_{L}~U_{M} |
| | C_{F4}~U_{L}C_{F4}~C_{M}C_{F}~U_{L}U_{F}~U_{M}~C_{L4}~U_{M} |
| | C_{F}~U_{L3}C_{F}~C_{M}C_{F}~U_{L3}U_{F}~U_{M3}~C_{L}~U_{M3} |
| | C_{F4}~U_{L}C_{F4}~C_{M}C_{F}~U_{L}U_{F3}~U_{M}~C_{L4}~U_{M} |
| | C_{F}~U_{L}C_{F4}~C_{M4}C_{F}~U_{L3}U_{F3}~U_{M3}~C_{L}~U_{M3} |
| | C_{F}~U_{L}C_{F4}~C_{M4}C_{F4}~U_{L3}U_{F3}~U_{M3}~C_{L}~U_{M3} |
| | C_{F}~U_{L12}C_{F}~C_{M}C_{F}~U_{L12}U_{F}~U_{M12}~C_{L}~U_{M12} |
| | C_{F4}~U_{L}C_{F4}~C_{M}C_{F}~U_{L}U_{F12}~U_{M}~C_{L4}~U_{M} |
| | C_{F}~U_{L}C_{F4}~C_{M4}C_{F}~U_{L12}U_{F12}~U_{M12}~C_{L}~U_{M12} |
| | C_{F}~U_{L}C_{F4}~C_{M4}C_{F4}~U_{L12}U_{F12}~U_{M12}~C_{L}~U_{M12} |
| | C_{F}~U_{L18}C_{F}~C_{M}C_{F}~U_{L18}U_{F}~U_{M18}~C_{L}~U_{M18} |
| | C_{F4}~U_{L}C_{F4}~C_{M}C_{F}~U_{L}U_{F18}~U_{M}~C_{L4}~U_{M} |
| | C_{F}~U_{L}C_{F4}~C_{M4}C_{F}~U_{L18}U_{F18}~U_{M18}~C_{L}~U_{M18} |
| | C_{F}~U_{L}C_{F4}~C_{M4}C_{F4}~U_{L18}U_{F18}~U_{M18}~C_{L}~U_{M18} |
| | C_{F4}~U_{L}C_{F4}~C_{M}C~U_{L}U_{F}~U_{M}~C_{L4}~U_{M} |
| | C_{F}~U_{L3}C_{F}~C_{M}C~U_{L3}U_{F}~U_{M3}~C_{L}~U_{M3} |
| | C_{F4}~U_{L}C_{F4}~C_{M}C~U_{L}U_{F3}~U_{M}~C_{L4}~U_{M} |
| | C_{F}~U_{L}C_{F4}~C_{M4}C~U_{L3}U_{F3}~U_{M3}~C_{L}~U_{M3} |
| | C_{F}~U_{L}C_{F4}~C_{M4}C₄~U_{L3}U_{F3}~U_{M3}~C_{L}~U_{M3} |
| | C_{F}~U_{L}L2C_{F}~C_{M}C~U_{L12}U_{F}~U_{M12}~C_{L}~U_{M12} |
| | C_{F4}~U_{L}C_{F4}~C_{M}C~U_{L}U_{F12}~U_{M}~C_{L4}~U_{M} |
| | C_{F}~U_{L}C_{F4}~C_{M4}C~U_{L12}U_{F12}~U_{M12}~C_{L}~U_{M12} |
| | C_{F}~U_{L}C_{F4}~C_{M4}C₄~U_{L12}U_{F12}~U_{M12}~C_{L}~U_{M12} |
| | C_{F}~U_{L18}C_{F}~C_{M}C~U_{L18}U_{F}~U_{M18}~C_{L}~U_{M18} |
| | C_{F4}~U_{L}C_{F4}~C_{M}C~U_{L}U_{F18}~U_{M}~C_{L4}~U_{M} |
| | C_{F}~U_{L}C_{F4}~C_{M4}C~U_{L18}U_{F18}~U_{M18}~C_{L}~U_{M18} |
| | C_{F}~U_{L}C_{F4}~C_{M4}C₄~U_{L18}U_{F18}~U_{M18}~C_{L}~U_{M18} |
| | C_{L}~U_{M}C_{L}~C_{M}C_{F}~U_{L}U_{F}~U_{L}~C_{F}~U_{L} |
| | C_{L}~U_{M}C_{L}~C_{M}C~U_{L}U_{F}~U_{L}~C_{F}~U_{L} |
| | C_{L}~A~U_{M}C_{L4}~C_{M}C_{F}~U_{L}U_{F}~U_{L}~C_{F4}~U_{L} |
| | C_{L}~U_{M3}C_{L}~C_{M}C_{F}~U_{L3}U_{F}~U_{L3}~C_{F}~U_{L3} |
| | C_{L4}~U_{M}C_{L}~A~C_{M}C_{F}~U_{L}U_{F3}~U_{L}~C_{F4}~U_{L} |
| | C_{L}~U_{M}C_{L4}~C_{M4}C_{F}~U_{L3}U_{F3}~U_{L3}~C_{F}~U_{L3} |
| | C_{L}~U_{M}C_{L4}~C_{M4}C_{F4}~U_{L3}U_{F3}~U_{L3}~C_{F}~U_{L3} |
| | C_{L}~U_{M12}C_{L}~C_{M}C_{F}~U_{L12}U_{F}~U_{L12}~C_{F}~U_{L12} |
| | C_{L4}~U_{M}C_{L4}~C_{M}C_{F}~U_{L}U_{F12}~U_{L}~C_{F4}~U_{L} |
| | C_{L}~U_{M}C_{L4}~C_{M4}C_{F}~U_{L12}U_{F12}~U_{L12}~C_{F}~U_{L12} |
| | C_{L}~U_{M}C_{L4}~C_{M4}C_{F4}~U_{L12}U_{F12}~U_{L12}~C_{F}~U_{L12} |
| | C_{L}~U_{M18}C_{L}~C_{M}C_{F}~U_{L18}U_{F}~U_{L18}~C_{F}~U_{L18} |
| | C_{L4}~U_{M}C_{L4}~C_{M}C_{F}~U_{L}U_{F18}~U_{L}~C_{F4}~U_{L} |
| | C_{L}~U_{M}C_{L4}~C_{M4}C_{F}~U_{L18}U_{F18}~U_{L18}~C_{F}~U_{L18} |
| | C_{L}~U_{M}C_{L4}~C_{M4}C_{F4}~U_{L18}U_{F18}~U_{L18}~C_{F}~U_{L18} |
| | C_{L4}~U_{M}C_{L4}~C_{M}C~U_{L}U_{F}~U_{L}~C_{F4}~U_{L} |
| | C_{L}~U_{M3}C_{L}~C_{M}C~U_{L3}U_{F}~U_{L3}~C_{F}~U_{L3} |
| | C_{L4}~U_{M}C_{L4}~C_{M}C~U_{L}U_{F3}~U_{L}~C_{F4}~U_{L} |
| | C_{F}~U_{M}C_{F4}~C_{M4}C~U_{L3}U_{F3}~U_{L3}~C_{F}~U_{L3} |
| | C_{L}~U_{M}C_{L4}~C_{M4}C₄~U_{L3}U_{F3}~U_{L3}~C_{F}~U_{L3} |
| | C_{L}~U_{M12}C_{L}~C_{M}C~U_{L12}U_{F}~U_{L12}~C_{F}~U_{L12} |
| | C_{L4}~U_{M}C_{L4}~C_{M}C~U_{L}U_{F12}~U_{L}~C_{F4}~U_{L} |
| | C_{L}~U_{M}C_{L4}~C_{M4}C~U_{L12}U_{F12}~U_{L12}~C_{F}~U_{L12} |
| | C_{L}~U_{M}C_{L4}~C_{M4}C₄~U_{L12}U_{F12}~U_{L12}~C_{F}~U_{L12} |
| | C_{L}~U_{M18}C_{L}~C_{M}C~U_{L18}U_{F}~U_{L18}~C_{F}~U_{L18} |
| | C_{L4}~U_{M}C_{L4}~C_{M}C~U_{L}U_{F18}~U_{L}~C_{F4}~U_{L} |
| | C_{L}~U_{M}C_{L4}~C_{M4}C~U_{L18}U_{F18}~U_{L18}~C_{F}~U_{L18} |
| | C_{L}~U_{M}C_{L4}~C_{M4}C₄~U_{L18}U_{F18}~U_{L18}~C_{F}~U_{L18} |
| | C_{F}~U_{L}C_{F}~C_{L}~C_{F}~U_{M}U_{F}~U_{M}~C_{L}~U_{M} |
| | C_{F}~U_{L}C_{F}~C_{L}~C~U_{M}U_{F}~U_{M}~C_{L}~U_{M} |
| | C_{F4}~U_{L}C_{F4}~C_{L}~C_{F}~U_{M}U_{F}~U_{M}~C_{L4}~U_{M} |
| | C_{F}~U_{F3}C_{F}~C_{L}~C_{F}~U_{M3}U_{F}~U_{M3}~C_{L}~U_{M3} |
| | C_{F4}~U_{L}C_{F4}~C_{L}~C_{F}~U_{M}U_{F3}~U_{M}~C_{L4}~U_{M} |
| | C_{F}~U_{L}C_{F4}~C_{L4}C_{F}~U_{M3}U_{F3}~U_{M3}~C_{L}~U_{M3} |
| | C_{F}~U_{L}C_{F4}~C_{L4}C_{F4}~U_{M3}U_{F3}~U_{M3}~C_{L}~U_{M3} |
| | C_{F}~U_{L12}C_{F}~C_{L}~C_{F}~U_{M12}U_{F}~U_{M12}~C_{L}~U_{M12} |
| | C_{F4}~U_{L}C_{F4}~C_{L}~C_{F}~U_{M}U_{F12}~U_{M}~C_{L4}~U_{M} |
| | C_{F}~U_{L}C_{F4}~C_{L4}C_{F}~U_{M12}U_{F12}~U_{M12}~C_{L}~U_{M12} |
| | C_{F}~U_{L}C_{F4}~C_{L4}C_{F4}~U_{M12}U_{F12}~U_{M12}~C_{L}~U_{M12} |
| | C_{F}~U_{L18}C_{F}~C_{L}~C_{F}~U_{M18}U_{F}~U_{M18}~C_{L}~U_{M18} |
| | C_{F4}~U_{L}C_{F4}~C_{L}~C_{F}~U_{M}U_{F18}~U_{M}~C_{L4}~U_{M} |
| | C_{F}~U_{L}C_{F4}~C_{L4}C_{F}~U_{M18}U_{F18}~U_{M18}~C_{L}~U_{M18} |
| | C_{F}~U_{L}C_{F4}~C_{L4}C_{F4}~U_{M18}U_{F18}~U_{M18}~C_{L}~U_{M18} |
| | C_{F4}~U_{L}C_{F4}~C_{L}~C~U_{M}U_{F}~U_{M}~C_{L4}~U_{M} |
| | C_{F}~U_{L3}C_{F}~C_{L}~C~U_{M3}U_{F}~U_{M3}~C_{L}~U_{M3} |
| | C_{F4}~U_{L}C_{F4}~C_{L}~C~U_{M}U_{F3}~U_{M}~C_{L4}~U_{M} |
| | C_{F}~U_{L}C_{F4}~C_{L4}C~U_{M3}U_{F3}~U_{M3}~C_{L}~U_{M3} |
| | C_{F}~U_{L}C_{F4}~C_{L4}C₄~U_{M3}U_{F3}~U_{M3}~C_{L}~U_{M3} |
| | C_{F}~U_{L12}C_{F}~C_{L}~C~U_{M12}U_{F}~U_{M12}~C_{L}~U_{M12} |
| | C_{F4}~U_{L}C_{F4}~C_{L}~C~U_{M}U_{F12}~U_{M}~C_{L4}~U_{M} |
| | C_{F}~U_{L}C_{F4}~C_{L4}C~U_{M12}U_{F12}~U_{M12}~C_{L}~U_{M12} |
| | C_{F}~U_{L}C_{F4}~C_{L4}C₄~U_{M12}U_{F12}~U_{M12}~C_{L}~U_{M12} |
| | C_{F}~U_{L18}C_{F}~C_{L}~C~U_{M18}U_{F}~U_{M18}~C_{L}~U_{M18} |
| | C_{F4}~U_{L}C_{F4}~C_{L}~C~U_{M}U_{F18}~U_{M}~C_{L4}~U_{M} |
| | C_{F}~U_{L}C_{F4}~C_{L4}C~U_{M18}U_{F18}~U_{M18}~C_{L}~U_{M18} |
| | C_{F}~U_{L}C_{F4}~C_{L4}C₄~U_{M18}U_{F18}~U_{M18}~C_{L}~U_{M18} |
| | C_{F}~U_{M}C_{L}~C_{M}C_{F}~U_{M}U_{L}~U_{M}~C_{L}~U_{M} |
| | C_{F}~U_{M}C_{L}~C_{M}C~U_{M}U_{L}~U_{M}~C_{L}~U_{M} |
| | C_{F4}~U_{M}C_{L4}~C_{M}C_{F}~U_{M}U_{L}~U_{M}~C_{L4}~U_{M} |
| | C_{F}~U_{M3}C_{L}~C_{M}C_{F}~U_{M3}U_{L}~U_{M3}~C_{L}~U_{M3} |
| | C_{F4}~U_{M}C_{L4}~C_{M}C_{F}~U_{M}U_{L3}~U_{M}~C_{L4}~U_{M} |
| | C_{F}~U_{M}C_{L4}~C_{M4}C_{F}~U_{M3}U_{L3}~U_{M3}~C_{L}~U_{M3} |
| | C_{F}~U_{M}C_{L4}~C_{M4}C_{F4}~U_{M3}U_{L3}~U_{M3}~C_{L}~U_{M3} |
| | C_{F}~U_{M12}C_{L}~C_{M}C_{F}~U_{M12}U_{L}~U_{M12}~C_{L}~U_{M12} |
| | C_{F4}~U_{M}C_{L4}~C_{M}C_{F}~U_{M}U_{L12}~U_{M}~C_{L4}~U_{M} |
| | C_{F}~U_{M}C_{L4}~C_{M4}C_{F}~U_{M12}U_{L12}~U_{M12}~C_{L}~U_{M12} |
| | C_{F}~U_{M}C_{L4}~C_{M4}C_{F4}~U_{M12}U_{L12}~U_{M12}~C_{F}~U_{M12} |
| | C_{F}~U_{M18}C_{L}~C_{M}C_{F}~U_{M18}U_{L}~U_{M18}~C_{L}~U_{M18} |
| | C_{F4}~U_{M}C_{L4}~C_{M}C_{F}~U_{M}U_{L18}~U_{M}~C_{L4}~U_{M} |
| | C_{F}~U_{M}C_{L4}~C_{M4}C_{F}~U_{M18}U_{L18}~U_{M18}~C_{L}~U_{M18} |
| | C_{F}~U_{M}C_{L4}~C_{M4}C_{F4}~U_{M18}U_{L18}~U_{M18}~C_{L}~U_{M18} |
| | C_{F4}~U_{M}C_{L4}~C_{M}C~U_{M}U_{L}~U_{M}~C_{L4}~U_{M} |
| | C_{F}~U_{M3}C_{L}~C_{M}C~U_{M3}U_{L}~U_{M3}~C_{L}~U_{M3} |
| | C_{F4}~U_{M}C_{L4}~C_{M}C~U_{M}U_{L3}~U_{M}~C_{L4}~U_{M} |
| | C_{F}~U_{M}C_{L4}~C_{M4}C~U_{M3}U_{L3}~U_{M3}~C_{L}~U_{M3} |
| | C_{F}~U_{M}C_{L4}~C_{M4}C₄~U_{M3}U_{L3}~U_{M3}~C_{L}~U_{M3} |
| | C_{F}~U_{M12}C_{L}~C_{M}C~U_{M12}U_{L}~U_{M12}~C_{L}~U_{M12} |
| | C_{F4}~U_{M}C_{L4}~C_{M}C~U_{M}U_{L12}~U_{M}~C_{L4}~U_{M} |
| | C_{F}~U_{M}C_{L4}~C_{M4}C~U_{M12}U_{L12}~U_{M12}~C_{L}~U_{M12} |
| | C_{F}~U_{M}C_{L4}~C_{M4}C₄~U_{M12}U_{L12}~U_{M12}~C_{L}~U_{M12} |
| | C_{F}~U_{M18}C_{L}~C_{M}C~U_{M18}U_{L}~U_{M18}~C_{L}~U_{M18} |
| | C_{F4}~U_{M}C_{L4}~C_{M}C~U_{M}U_{L18}~U_{M}~C_{L4}~U_{M} |
| | C_{F}~U_{M}C_{L4}~C_{M4}C~U_{M18}U_{L18}~U_{M18}~C_{L}~U_{M18} |
| | C_{F}~U_{M}C_{L4}~C_{M4}C₄~U_{M18}U_{L18}~U_{M18}~C_{L}~U_{M18} |

### Table 20

### Examples of Murine/Human miR-124 ags-MiR Compositions Based on Modular Design

### A. Endogenous Guide Strand Sequence;

The murine/human miR-124 guide strand sequence based on miRBase accession numbers MI0000150 and MI0000443 minus the overhang is as follows:

UAAGGCACGCGGUGAAUGC

B. Select Murine/Human MiR-124 ss-RNAi Compositions Designed and Evaluated by Chorn et al., RNA 18: 1796-804, 2012.

The following two ss-RNAi mimics of miR-124 are examples of those evaluated by Chorn et al.

The first example is based on the endogenous guide strand where the endogenous overhang is replaced by U_{M}U_{M}:

P-U_{F}A_{F}A_{F}G_{F}G_{F}C_{F}A_{F}C_{F}G_{F}C_{F}G_{F}G_{F}U_{F}G_{F}A_{F}A_{F}U_{F}G_{F}C_{F}C_{F}U_{M}U_{M}

The second example is based on the modular approach and it incorporates seed vehicle 9y_2 that is shown in Table 19A:

P-U_{F}A_{F}A_{F}G_{F}G_{F}C_{F}A_{F}C_{F}G_{F}C_{F}G_{F}C_{F}U_{F}U_{F}C_{F}U_{F}C_{F}U_{F}U_{F}U_{M}U_{M}

C. Application of AGSD to these Modular Components and Compounds:

As for any ss-RNAi the endogenous miR-124 sequence can be modified for basic nuclease resistance and by AGSD. Further it can have any of the overhang precursors provided herein.

The nine-nucleoside long 5′-end module containing the miR-124 sequence used by Chorn et al. (2012) is P-U_{F}A_{F}A_{F}G_{F}G_{F}C_{F}A_{F}C_{F}. Examples of this 5′-end module as modified by AGSD are provided in Table 20A while examples with a higher level of basic nuclease resistance than used by Chorn et al. are provided in Table 20B.

Any of the 5′-end nucleosides (P-U_{M}) shown in the examples of agsRNAi compounds in

Table 20A and 20B can be replaced with any of those provided for herein including where one of the 5′end nucleosides is selected from the group consisting of P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{V}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 20A or 20B with an L modification(s) found in one or more of those in positions 2-9 can have any given L or set of Ls replaced with any of those sugar or sugar analogs provided for herein for positions 2-19 including one or more of them independently selected from the group consisting of: S, J, W, V, Q, Y, O, T and cet (i.e., if multiple Ls are replaced the selected replacements do not all have to be the same sugar, sugar analog or sugar substitute).

Any of the 5′-end modules shown in Table 20A can be used with any seed vehicle shown in Table 19B while any of the 5′-end modules shown in Table 20B can be used with any seed vehicle shown in Table 19C. Further, any of the resulting compounds can have any of the overhang precursors provided for herein.

TABLE 20A
Examples of the Application of AGSD to the 5′-end
Module for miR-124 used by Chorn et al. (2012)
Compositions of Matter Suitable for Use in
vitro or in a Subject with a Protective Carrier
(5′-3′)

| | |
|---|---|
| P-U_{F}A_{F}A_{F}G_{F}G_{F}C_{F}A_{F}C_{F}G_{L} | P-U_{F}A_{F}A_{L}G_{F}G_{L}C_{F}A_{F}C_{F}G_{L} |
| P-U_{F}A_{F}A_{F}G_{F}G_{F}C_{F4}A_{F}C_{F4}G_{F} | P-U_{F}A_{F}A_{L}G_{F}G_{L}C_{F}A_{L}C_{F}G_{F} |
| P-U_{F}A_{F}A_{F}G_{F}G_{F}C_{F}A_{F}C_{F}G_{L} | P-U_{F}A_{F}A_{L}G_{F}G_{L}C_{F}A_{L}C_{F}G_{L} |
| P-U_{F}A_{F}A_{F}G_{F}G_{F}C_{F4}A_{F}C_{F4}G_{L} | P-U_{F}A_{F}A_{F}G_{L}G_{F}C_{L}A_{F}C_{L}G_{F} |
| P-U_{F}A_{L}A_{F}G_{F}G_{F}C_{F}A_{F}C_{F}G_{F} | P-U_{F}A_{L}A_{F}G_{L}G_{F}C_{L}A_{F}C_{L}G_{F} |
| P-U_{F}A_{F}A_{L}G_{F}G_{F}C_{F}A_{F}C_{F}G_{F} | P-U_{F}A_{F}A_{F}G_{L}G_{F}C_{F}A_{F}C_{L}G_{F} |
| P-U_{F}A_{F}A_{F}G_{L}G_{F}C_{F}A_{F}C_{F}G_{F} | P-U_{F}A_{F}A_{F}G_{F}G_{F}C_{L4}A_{F}C_{L4}G_{F} |
| P-U_{F}A_{F}A_{F}G_{F}G_{L}C_{F}A_{F}C_{F}G_{F} | P-U_{F}A_{F}A_{L}G_{F}G_{F}C_{L4}A_{F}C_{L4}G_{F} |
| P-U_{F}A_{F}A_{F}G_{F}G_{F}C_{F}A_{L}C_{F}G_{F} | P-U_{F}A_{L}A_{F}G_{L}G_{F}C_{L4}A_{F}C_{L4}G_{F} |
| P-U_{F}A_{F}A_{F}G_{F}G_{F}C_{L}A_{F}C_{F}G_{F} | P-U_{F}A_{F}A_{F}G_{L}G_{F}C_{F}A_{F}C_{F4}G_{L} |
| P-U_{F}A_{F}A_{F}G_{F}G_{F}C_{F}A_{F}C_{L}G_{F} | P-U_{F}A_{F}A_{L}G_{F}G_{L}C_{F}A_{F}C_{F4}G_{L} |
| P-U_{F}A_{F}A_{F}G_{F}G_{F}C_{F}A_{L}C_{F}G_{L} | P-U_{F}A_{F}A_{F}G_{F}G_{F}C_{F4}A_{L}C_{F}G_{L} |
| P-U_{F}A_{F}A_{F}G_{F}G_{F}C_{F}A_{L}C_{F4}G_{L} | P-U_{F}A_{F}A_{F}G_{F}G_{F}C_{F4}A_{L}C_{F4}G_{L} |

TABLE 20B
Examples of the Application of AGSD to the 5′-end Module
or miR-124 used by Chorn et al. (2012) as Adjusted for
Use in Subjects with or without a Protective Carrier
Compositions of Matter Suitable for Administration to a
Subject with or without a Protective Carrier and for Any
Other use as Provided for Herein
(5′-3′)

| | |
|---|---|
| P-U_{M}~A_{F}~A_{M}G_{F}~G_{M}C_{F}~A_{M}C_{F}~G_{L} | P-U_{M}~A_{F}~A_{L}G_{F}~G_{L}C_{F}~A_{M}C_{F}~G_{L} |
| P-U_{M}~A_{F}~A_{M}G_{F}~G_{M}C_{F4}~A_{M}C_{F4}~G_{M} | P-U_{M}~A_{F}~A_{L}G_{F}~G_{L}C_{F}~A_{L}C_{F}~G_{M} |
| P-U_{M}~A_{F}~A_{M}G_{F}~G_{M}C_{F4}~A_{M}C_{F}~G_{L} | P-U_{M}~A_{F}~A_{L}G_{F}~G_{L}C_{F}~A_{L}C_{F}~G_{L} |
| P-U_{M}~A_{F}~A_{M}G_{F}~G_{M}C_{F4}~A_{M}C_{F4}~G_{L} | P-U_{M}~A_{F}~A_{M}G_{L}~G_{M}C_{L}~A_{M}C_{L}~G_{M} |
| P-U_{M}~A_{L}~A_{M}G_{F}~G_{M}C_{F}~A_{M}C_{F}~G_{M} | P-U_{M}~A_{L}~A_{M}G_{L}~G_{M}C_{L}~A_{M}C_{L}~G_{M} |
| P-U_{M}~A_{F}~A_{L}G_{F}~G_{M}C_{F}~A_{M}C_{F}~G_{M} | P-U_{M}~A_{F}~A_{M}G_{L}~G_{M}C_{F}~A_{M}C_{L}~G_{M} |
| P-U_{M}~A_{F}~A_{M}G_{L}~G_{M}C_{F}~A_{M}C_{F}~G_{M} | P-U_{M}~A_{F}~A_{M}G_{F}~G_{M}C_{L4}~A_{M}C_{L4}~G_{M} |
| P-U_{M}~A_{F}~A_{M}G_{F}~G_{L}C_{F}~A_{M}C_{F}~G_{M} | P-U_{M}~A_{F}~A_{L}G_{F}~G_{M}C_{L4}~A_{M}C_{L4}~G_{M} |
| P-U_{M}~A_{F}~A_{M}G_{F}~G_{M}C_{F}~A_{L}C_{F}~G_{M} | P-U_{M}~A_{L}~A_{M}G_{L}~G_{M}C_{L4}~A_{M}C_{L4}~G_{M} |
| P-U_{M}~A_{F}~A_{M}G_{F}~G_{M}C_{L}~A_{M}C_{F}~G_{M} | P-U_{M}~A_{F}~A_{M}G_{L}~G_{M}C_{F}~A_{M}C_{F4}~G_{L} |
| P-U_{M}~A_{F}~A_{M}G_{F}~G_{M}C_{F}~A_{M}C_{L}~G_{M} | P-U_{M}~A_{F}~A_{L}G_{F}~G_{L}C_{F}~A_{M}C_{F4}~G_{L} |
| P-U_{M}~A_{F}~A_{M}G_{F}~G_{M}C_{F}~A_{L}C_{F}~G_{L} | P-U_{M}~A_{F}~A_{M}G_{F}~G_{M}C_{F4}~A_{L}C_{F}~G_{L} |
| P-U_{M}~A_{F}~A_{M}G_{F}~G_{M}C_{F}~A_{L}C_{F4}~G_{L} | P-U_{M}~A_{F}~A_{M}G_{F}~G_{M}C_{F4}~A_{L}C_{F4}~G_{L} |

### Table 21

### Murine/Human MiR-122 ags-MiR Compositions Based on the Endogenous Sequence or on Modular Design

### A. Endogenous Guide Strand Sequence;

The murine/human miR-122 guide strand based on miRBase accession numbers MI0000256 and MI0000442 minus the overhang is as follows:

UGGAGUGUGACAAUGGUGU

B. Select Murine/Human MiR-122 ss-RNAi Compositions Designed and Evaluated by Chorn et al., RNA 18: 1796-804, 2012.

The following two ss-RNAi mimics of miR-122 are examples of those evaluated by Chorn et al.

The first example is based on the endogenous guide strand where the endogenous overhang is replaced by U_{M}U_{M}:

Modified Endogenous miR-122 Compound:

P-U_{F}G_{F}G_{F}A_{F}G_{F}U_{F}G_{F}U_{F}G_{F}A_{F}C_{F}A_{F}A_{F}U_{F}G_{F}G_{F}U_{F}G_{F}U_{F}U_{M}U_{M}

The second example is based on the modular approach and it incorporates seed vehicle 9y_2 that is shown in Table 19A:

P-U_{F}G_{F}G_{F}A_{F}G_{F}U_{F}G_{F}U_{F}G_{F}C_{F}G_{F}C_{F}U_{F}U_{F}C_{F}U_{F}C_{F}U_{F}U_{F}U_{M}U_{M}

C. Application of AGSD to these Modular Components and Compounds:

As for any ss-RNAi the endogenous miR-122 sequence can be modified for basic nuclease resistance and by AGSD. Further it can have any of the overhang precursors provided herein.

The nine-nucleoside long 5′-end module containing the miR-122 sequence used by Chorn et al. (2012) is P-U_{F}G_{F}G_{F}A_{F}G_{F}U_{F}G_{F}U_{F}G_{F}. Examples of this 5′-end module as modified by AGSD are provided in Table 21A while examples with a higher level of basic nuclease resistance are provided in Table 21B.

Any of the 5′-end nucleosides (P-U_{M}) shown in the examples of agsRNAi compounds in these two tables can be replaced with any of those provided for herein including where one of the 5′end nucleosides is selected from the group consisting of P-G_{F}, P-G_{M}, P-G_{N}, P-G_{L}, P-G_{Z}, P-G_{H}, P-G_{B}, P-G_{K}, P-G_{Q}, P-G_{S}, P-G_{T}, P-G_{W}, P-G_{Y}, P-G_{O}, P-C_{F}, P-C_{M}, P-C_{N}, P-C_{L}, P-C_{Z}, P-C_{H}, P-C_{B}, P-C_{K}, P-C_{Q}, P-C_{S}, P-C_{T}, P-C_{W}, P-C_{V}, P-C_{O}, P-U_{F}, P-U_{M}, P-U_{L}, P-U_{Z}, P-U_{H}, P-U_{B}, P-U_{K}, P-U_{Q}, P-U_{S}, P-U_{T}, P-U_{W}, P-U_{V}, P-U_{O}, P-T_{L}, P-T_{H}, 5′-VP-U_{Z}, 5′-VP-U_{K}, 5′-VP-U_{Q}, 5′-VP-U_{B}, 5′-VP-Tₘₒₑ, 5′-VP-T_{E}, 5′-VP-U_{E}, 5′-VP-T_{Z}, 5′-VP-T_{K}, 5′-VP-T_{Q} and 5′-VP-T_{B}.

Any of the nucleosides in Table 21A or 21B with an L modification(s) found in one or more of those in positions 2-9 can have any given L or set of Ls replaced with any of those sugar, sugar analogs or sugar substitutes provided for herein for positions 2-19 including one or more of those independently selected from the group consisting of: S, J, W, V, Q, Y, O, T and cet (i.e., if multiple Ls are replaced the selected replacements do not all have to be the same sugar, sugar analog or sugar substitute).

Any of the 5′-end modules shown in Table 21A can be used with any seed vehicle shown in Table 19B while any of the 5′-end modules shown in Table 21B can be used with any seed vehicle shown in Table 19C. Further any of the resulting compounds can have any of the overhang precursors provided for herein.

TABLE 21A
Examples of the Application of AGSD to the 5′-end
Module for miR-122 used by Chorn et al. (2012)
Compositions of Matter Suitable for Use in vitro or
in a Subject with a Protective Carrier
(5′-3′)

| | |
|---|---|
| P-U_{F}G_{F}G_{F}A_{F}G_{F}U_{F3}G_{F}U_{F3}G_{F} | P-U_{F}G_{F}G_{L}A_{F}G_{F}U_{L}G_{F}U_{F}G_{F} |
| P-U_{F}G_{L}G_{F}A_{F}G_{L}U_{F}G_{F}U_{F}G_{L} | P-U_{F}G_{F}G_{L}A_{F}G_{F}U_{L3}G_{F}U_{F3}G_{F} |
| P-U_{F}G_{L}G_{F}A_{F}G_{L}U_{F3}G_{F}U_{F3}G_{L} | P-U_{F}G_{F}G_{L}A_{L}G_{F}U_{F}G_{F}U_{L}G_{F} |
| P-U_{F}G_{F}G_{F}A_{F}G_{F}U_{F}G_{L}U_{F}G_{L} | P-U_{F}G_{F}G_{L}A_{L}G_{F}U_{F3}G_{F}U_{L3}G_{F} |
| P-U_{F}G_{F}G_{F}A_{F}G_{F}U_{F3}G_{L}U_{F3}G_{L} | P-U_{F}G_{F}G_{L}A_{F}G_{L}U_{F}G_{F}U_{F}G_{L} |
| | P-U_{F}G_{F}G_{L}A_{F}G_{L}U_{F3}G_{F}U_{F3}G_{L} |
| P-U_{F}G_{F}G_{F}A_{F}G_{F}U_{F12}G_{F}U_{F12}G_{F} | P-U_{F}G_{F}G_{L}A_{F}G_{F}U_{L}G_{F}U_{F}G_{F} |
| P-U_{F}G_{L}G_{F}A_{F}G_{L}U_{F}G_{F}U_{F}G_{L} | P-U_{F}G_{F}G_{L}A_{F}G_{F}U_{L12}G_{F}U_{F12}G_{F} |
| P-U_{F}G_{L}G_{F}A_{F}G_{L}U_{F12}G_{F}U_{F12}G_{L} | P-U_{F}G_{F}G_{L}A_{L}G_{F}U_{F}G_{F}U_{L}G_{F} |
| P-U_{F}G_{F}G_{F}A_{F}G_{F}U_{F}G_{L}U_{F}G_{L} | P-U_{F}G_{F}G_{L}A_{L}G_{F}U_{F12}G_{F}U_{L12}G_{F} |
| P-U_{F}G_{F}G_{F}A_{F}G_{F}U_{F12}G_{L}U_{F12}G_{L} | P-U_{F}G_{F}G_{L}A_{F}G_{L}U_{F}G_{F}U_{F}G_{L} |
| | P-U_{F}G_{F}G_{L}A_{F}G_{L}U_{F12}G_{F}U_{F12}G_{L} |
| P-U_{F}G_{F}G_{F}A_{F}G_{F}U_{F18}G_{F}U_{F18}G_{F} | P-U_{F}G_{F}G_{L}A_{F}G_{F}U_{L}G_{F}U_{F}G_{F} |
| P-U_{F}G_{L}G_{F}A_{F}G_{L}U_{F}G_{F}U_{F}G_{L} | P-U_{F}G_{F}G_{L}A_{F}G_{F}U_{L18}G_{F}U_{F18}G_{F} |
| P-U_{F}G_{L}G_{F}A_{F}G_{L}U_{F18}G_{F}U_{F18}G_{L} | P-U_{F}G_{F}G_{L}A_{L}G_{F}U_{F}G_{F}U_{L}G_{F} |
| P-U_{F}G_{F}G_{F}A_{F}G_{F}U_{F}G_{L}U_{F}G_{L} | P-U_{F}G_{F}G_{L}A_{L}G_{F}U_{F18}G_{F}U_{L18}G_{F} |
| P-U_{F}G_{F}G_{F}A_{F}G_{F}U_{F18}G_{L}U_{F18}G_{L} | P-U_{F}G_{F}G_{L}A_{F}G_{L}U_{F}G_{F}U_{F}G_{L} |
| | P-U_{F}G_{F}G_{L}A_{F}G_{L}U_{F18}G_{F}U_{F18}G_{L} |

TABLE 21B
Examples of the Application of AGSD to the 5′-end Module
for miR-122 used by Chorn et al. (2012) as Adjusted for Use
in Subjects with or without a Protective Carrier
Compositions of Matter Suitable for Administration to a
Subject with or without a Protective Carrier and for Any
Other use as Provided for Herein
(5′-3′)

| | |
|---|---|
| P-U_{M}~G_{F}~G_{M}A_{F}~G_{M}U_{F3}~G_{M}U_{F3}~G_{M} | P-U_{M}~G_{F}~G_{M}A_{L}~G_{M}U_{F}~G_{M}U_{L}~G_{M} |
| P-U_{M}~G_{L}~G_{M}A_{F}~G_{L}U_{F}~G_{M}U_{F}~G_{L} | P-U_{M}~G_{F}~G_{M}A_{L}~G_{M}U_{F3}~G_{M}U_{L3}~G_{M} |
| P-U_{M}~G_{L}~G_{M}A_{F}~G_{L}U_{F3}~G_{M}U_{F3}~G_{L} | P-U_{M}~G_{F}~G_{L}A_{F}~G_{L}U_{F}~G_{M}U_{F}~G_{L} |
| P-U_{M}~G_{F}~G_{M}A_{F}~G_{M}U_{F}~G_{L}U_{F}~G_{L} | P-U_{M}~G_{F}~G_{L}A_{F}~G_{L}U_{F3}~G_{M}U_{F3}~G_{L} |
| P-U_{M}~G_{F}~G_{M}A_{F}~G_{M}U_{F3}~G_{L}U_{F3}~G_{L} | P-U_{M}~G_{F}~G_{M}A_{F}~G_{M}U_{F}~G_{M}U_{F}~G_{M} |
| P-U_{M}~G_{F}~G_{L}A_{F}~G_{M}U_{L}~G_{M}U_{F}~G_{M} | P-U_{M}~G_{F}~G_{M}A_{F}~G_{M}U_{F}~G_{M}U_{F}~G_{M} |
| P-U_{M}~G_{F}~G_{L}A_{F}~G_{M}U_{L3}~G_{M}U_{F3}~G_{M} | |
| P-U_{M}~G_{F}~G_{M}A_{F}~G_{M}U_{F12}~G_{M}U_{F12}~G_{M} | P-U_{M}~G_{F}~G_{M}A_{L}~G_{M}U_{F}~G_{M}U_{L}~G_{M} |
| P-U_{M}~G_{L}~G_{M}A_{F}~G_{L}U_{F}~G_{M}U_{F}~G_{L} | P-U_{M}~G_{F}~G_{M}A_{L}~G_{M}U_{F12}~G_{M}U_{L12}~G_{M} |
| P-U_{M}~G_{L}~G_{M}A_{F}~G_{L}U_{F12}~G_{M}U_{F12}~G_{L} | P-U_{M}~G_{F}~G_{L}A_{F}~G_{L}U_{F}~G_{M}U_{F}~G_{L} |
| P-U_{M}~G_{F}~G_{M}A_{F}~G_{M}U_{F}~G_{L}U_{F}~G_{L} | P-U_{M}~G_{F}~G_{L}A_{F}~G_{L}U_{F12}~G_{M}U_{F12}~G_{L} |
| P-U_{M}~G_{F}~G_{M}A_{F}~G_{M}U_{F12}~G_{L}U_{F12}~G_{L} | |
| P-U_{M}~G_{F}~G_{L}A_{F}~G_{M}U_{L}~G_{M}U_{F}~G_{M} | |
| P-U_{M}~G_{F}~G_{L}A_{F}~G_{M}U_{L12}~G_{M}U_{F12}~G_{M} | |
| P-U_{M}~G_{F}~G_{M}A_{F}~G_{M}U_{F18}~G_{M}U_{F18}~G_{M} | P-U_{M}~G_{F}~G_{M}A_{L}~G_{M}U_{F}~G_{M}U_{L}~G_{M} |
| P-U_{M}~G_{L}~G_{M}A_{F}~G_{L}U_{F}~G_{M}U_{F}~G_{L} | P-U_{M}~G_{F}~G_{M}A_{L}~G_{M}U_{F18}~G_{M}U_{L18}~G_{M} |
| P-U_{M}~G_{L}~G_{M}A_{F}~G_{L}U_{F18}~G_{M}U_{F18}~G_{L} | P-U_{M}~G_{F}~G_{L}A_{F}~G_{L}U_{F}~G_{M}U_{F}~G_{L} |
| P-U_{M}~G_{F}~G_{M}A_{F}~G_{M}U_{F}~G_{L}U_{F}~G_{L} | P-U_{M}~G_{F}~G_{L}A_{F}~G_{L}U_{F18}~G_{M}U_{F18}~G_{L} |
| P-U_{M}~G_{F}~G_{M}A_{F}~G_{M}U_{F18}~G_{L}U_{F18}~G_{L} | |
| P-U_{M}~G_{F}~G_{L}A_{F}~G_{M}U_{L}~G_{M}U_{F}~G_{M} | |
| P-U_{M}~G_{F}~G_{L}A_{F}~G_{M}U_{L18}~G_{M}U_{F18}~G_{M} | |

The following examples are provided to illustrate certain embodiments of the invention. They are not intended to limit the invention in any way.

### Example I

### Applications for ags-IMiRs

MiRNAs have been shown to have wide ranging effects on gene expression. In certain instances, these effects are detrimental and related to certain pathologies. Accordingly, specific miRNA inhibitors which target such miRNAs for degradation are highly desirable. The present inventor has devised strategies for the synthesis of miRNA inhibitors (agslMiRs) suitable for in vivo delivery which exhibit enhanced stability, the ability to form active RNAi triggers in cells, which act in turn to inhibit the activity of endogenous miRNAs associated with disease. These design paradigms and the resulting miRNA inhibitors are described herein above.

Table 22 provides a listing of some of the medical uses of the ags-IMiRs directed to the indicated miRNAs. The methods of the present invention, however, can be used to generate ags-IMiRs against any miRNA. Methods for administration of the oligos of the invention are provided in detail above.

TABLE 22
MICRORNA TARGETS FOR INHIBITION BY ags-IMiRs
AND COMMERCIAL APPLICATIONS

| MicroRNA | Medical Conditions to be Treated using the |
|---|---|
| Targets | ags-IMiR Compounds of the Invention |
| miR-24 | Treat cancer including hormone resistant prostate |
| miR-29a | Inhibit pathologic apoptosis including that due to ischemia |
| | reperfusion injury such as occurs after the removal of a clot |
| miR-29b | Inhibit pathologic apoptosis |
| miR-29c | Inhibit pathologic apoptosis including that due to ischemia |
| | reperfusion injury such as occurs after the removal of a clot |
| miR-33 | Raise good cholesterol (HDL) levels |
| miR-34a | Myocardial infarction |
| miR-122 | Hepatitis C |
| miR-155 | Arthritis; Autoimmune inflammation including that |
| | associated with cystic fibrosis; Atopic dermatitis |
| miR-208a | Chronic heart failure |

Elevated levels of miR-21, for example, occur in numerous cancers where it promotes oncogenesis at least in part by preventing the translation and accumulation of PDCD4. Another example is miR-122 a liver specific miRNA that promotes replication of the hepatitis C virus.

Compared to antisense oligos that engender catalytic activity against their targets, such as those that are RNase H dependent, the antisense oligos that function as competitive inhibitors must be used at substantially higher concentrations. In vivo various tissues take up oligos in widely ranging amounts. For example, liver and kidney take up relatively large amounts while resting lymphocytes, testis, skeletal muscle the CNS and other tissues take up much smaller amounts. Further, antisense oligos that have a competitive inhibitor function have been shown to perform poorly in tissues that do not avidly take up oligos. Therefore, it would be highly desirable to have oligonucleotide based miRNA inhibitors that have a catalytic activity against such targets so that a wider range of tissues types can be subject to efficient miRNA inhibition. The present invention provides a solution to this pressing need.

### Example II

### Examples of Applications for miRNA Mimics

Table 23 below provides a listing of miRNAs for which examples of specific ss-MiRcompounds have been provided herein. The methods of the present invention can be used to mimic any endogenous miRNA, to improve on the mRNA type silencing pattern of an endogenous miRNA for commercial purposes and can be used to generate designer novel miRNA-like compounds.

TABLE 23
MICRORNAS MIMICKED BY ags-MiRs AND COMMERCIAL
APPLICATIONS

| MicroRNA | |
|---|---|
| Mimicked | Medical Conditions to be Treated using the ags-MiR |
| by ss-MiR | Compounds of the Invention |
| Let-7i and | Cancer |
| Let-7 | |
| family | |
| generally | |
| miR-24-1 | Ischemia reperfusion injury including that associated with |
| | myocardial infarction; Cardiac fibrosis; Diabetes |
| miR-24-2 | Ischemia reperfusion injury including that associated with |
| | myocardial infarction; Cardiac fibrosis; Diabetes |
| miR-26a-1 | Cancer including liver, head and neck, breast |
| miR-26a-2 | Cancer including liver, head and neck, breast |
| miR-29a | Fibrosis including liver, lung, kidney and heart; Systemic |
| | sclerosis; Cancers including lung, liver, chronic lymphocytic |
| | leukemia; Osteoporosis; Systemic sclerosis; |
| miR-29b-1 | Fibrosis including liver, lung, kidney and heart; Systemic |
| | sclerosis; Cancers including lung, liver, colon breast, chronic |
| | lymphocytic leukemia, acute myeloid leukemia |
| miR-29b-2 | Fibrosis including liver, lung, kidney and heart; Systemic |
| | sclerosis; Cancers including lung, liver, colon, breast, |
| | rhabdomyosarcoma, chronic lymphocytic leukemia, |
| | acute myeloid leukemia; |
| miR-29c | Fibrosis including liver, lung, kidney and heart; Systemic |
| | sclerosis; Cancers including lung, liver, rhabdomyosarcoma, |
| | chronic lymphocytic leukemia; |
| miR-34a | Cancer including prostate, ovarian, non-small cell lung cancer, |
| | pancreatic cancer, stomach cancer, retinoblastoma and chronic |
| | lymphocytic leukemia; |
| miR-34b | Cancer including prostate, ovarian, non-small cell lung cancer, |
| | pancreatic cancer, stomach cancer, retinoblastoma and chronic |
| | lymphocytic leukemia; |
| miR-34c | Cancer including prostate, ovarian, non-small cell lung cancer, |
| | pancreatic cancer, stomach cancer, retinoblastoma and chronic |
| | lymphocytic leukemia; |
| miR-122 | Cancer including liver, lung and cervical; |
| miR-146a | Atherosclerosis |
| miR-203 | Sensitize cancers with mutant p53 including colon cancer to |
| | chemotherapy including taxanes |
| miR-214 | Nerve regeneration; Diabetes including type 2; |
| miR-499 | Myocardial infarction including the ischemia-reperfusion injury |
| | related to reversing vessel occlusion; |

It is now well established that post-transcriptional gene silencing (PTGS) by miRNA and other RNAi-associated pathways represents an essential layer of complexity to gene regulation. Gene silencing using RNAi additionally demonstrates huge potential as a therapeutic strategy for eliminating gene expression associated with the pathology underlying a number of different disorders.

A number of conventional miRNA compounds closely based on their endogenous miRNA counterparts are provided herein as putative therapeutic agents. Cancer is one area of focus since it has been found that several different miRNAs are expressed at very low levels in cancer cells compared to their normal counterparts. Further, it has been shown that replacing these miRNAs can have profound anticancer effects. Several specific examples are provided in the Table.

The miRNA mimics provided should also be effective in cell culture in vitro. They can be tested in single stranded form as described above or in the presence of a passenger strand, also described in detail above. In this approach, the first strand can be transfected into the target cells following by subsequent transfection of the second strand after a certain time frame has elapsed. This method should facilitate drug discovery efforts, target validation and also provide the means to reduce or eliminate any undesirable off target effects.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A single strand oligoribonucleotide ags-siRNA or ags-IMiR compound for modulating the expression and/or function of at least one target nucleic acid sequence expressed in a cell, comprising:
a) a nucleoside in position 1 and an associated linkage with the nucleoside in position 2 comprising modifications that promote binding of the compound to an argonaute protein; and
b) 19 nucleosides operably linked to said first nucleoside, said nucleosides comprising modifications in positions 2-19 that increase nuclease resistance; wherein at least 4 AGSD modifications are present;
c) 1, 2, 3, or 4 optional overhang precursor units; and
d) a region of complementary base pairing with the target nucleic acid that is at least 15 consecutive nucleosides in length beginning at position 2 and extending at least through position 16;
wherein the compound exhibits a maximal plateau level of activity in a dose response curve against the target that constitutes at least a 50% change in expression and/or function of the target and said level of change in activity is at least 20 percentage points greater than the maximal level of activity obtained using an ssRNAi which lacks said AGSD modifications of the same sequence and same 5′end moiety, if any.

2. A method for modulating expression and or function of a target nucleic acid in a target cell comprising administration of an effective amount of the compound of claim 1 wherein said ags-siRNA is directed to a target selected from the group consisting of pre-mRNA, mRNA, lncRNA, promoter associated RNA, enhancer RNA, snoRNA, piRNA, xiRNA, sdRNA, moRNA, MSY-RNA, tel-sRNA, crasiRNA, endogenous antisense RNA, a promoters, an enhancers and a suppressors.

3. The method of claim 2, wherein expression and or function of an miRNA target is modulated and said compound is an ags-IMiR.

4. The method of claim 2 wherein said target cells are selected from the group consisting of a cell line; a tissue sample from an organ, gland or neoplastic growth; an enriched sample of parenchymal cells from an organ, gland or neoplastic growth; epithelial tissue including simple, stratified, pseudostratified and transitional epithelium; connective tissues, reticular, adipose, blood and lymphoid tissues; of nervous tissue isolated from brain, spinal cord, ganglion, and nerve and, neuronal cells, glial cells, skeletal muscle cells, cardiac muscle cells and smooth muscle cells.

5. The compound of claim 1 wherein said the nucleoside in position 1 and the associated linkage with the nucleoside in position 2 promote binding of the compound to an argonaute protein and comprise one or more sugar modifications consisting of 2′-fluoro, 2′-O-methyl, 2′-methoxyethyl, 2′-deoxyribose, LNA, FANA, 4'S-FANA, ALN, AENA, CENA, HM, HNA, EA, F-CeNA, CeNA, UNA, CRN R monomer and CRN Q monomer and one or more linkage modifications selected from phosphodiester, phosphorothioate, N3′ phosphoramidate and amide.

6. The compound of claim 1 wherein said overhang precursor units are selected from the group consisting of ˜GL˜UL˜UX, ˜UL˜UL˜UX, ˜CL˜UL˜UX, ˜GL˜CL˜UX, ˜AL˜GL˜UX, ˜AL˜GL˜CX, ˜AL˜AL˜AX, ˜AL˜GL˜AX, ˜GL˜AL˜AX, ˜CL˜GL˜CX, ˜GL˜GL˜CX and ˜GL˜GL˜UX where the subscripts represent sugar modifications indicated by L are selected from the group consisting of L, F, J, W, V, Y, T, TL and I and the modifications indicated by X are selected from the group consisting of ribose, 2′-fluoro, or 2′-O-methyl.

7. The compound of claim 6 wherein said overhang precursors are linked to the nucleoside in position 19 and to each other by a linkage selected from the group consisting of phosphodiester, phosphorothioate, N3′ phosphoramidate and amide.

8. The method of claim 2 wherein said compound and comparator ssRNAi are delivered to a cell line or a primary parenchymal cell in vitro via a method selected from the group consisting of transfection, electroporation and gymnosis.

9. The compound of claim 1 wherein said compound and comparator ssRNAi comprise an enveloping protective carrier.

10. The compound of claim 9 wherein said nuclease resistance is achieved via inclusion of one or more modifications consisting of:
a) all of the nucleosides in positions 2-19 are 2′-fluoro and all of the linkages phosphodiester;
b) nucleosides in positions 2-19 are 2′-fluoro alternated with 2′-O-methyl nucleosides with the 2′-fluoro being in the even numbered positions,
c) inclusion of a 2′-O-methyl modification at nucleoside position 2 and two contiguous nucleosides in the region defined by positions 3-13 have a 2′-fluoro modified sugar so that when the alternating pattern of single 2′-O-methyl with single 2′-fluoro modified sugar is continued the 2′-fluoro modification will fall on nucleoside positions 14 and 16;
d) inclusion of a ribose or HM at position 14 and optionally at position 16; and
e) inclusion of phosphorothioate linkages at sites 2-3, and an overhang precursor where the units are linked to each other and to nucleoside 19 by phosphorothioates.

11. The method of claim 2 wherein said compound and comparator ssRNAi are administered to cells in a subject without an enveloping protective carrier.

12. The method of claim 11 wherein said nuclease resistance is achieved via inclusion of 2′-fluoro alternating with 2′-O-methyl nucleosides in positions 2-19 with the 2′-fluoro being in the even numbered positions where phosphorothioate linkages occur between nucleoside positions 2-3, 4-5, 6-7, 8-9, 10-11, 12-13, 14-15, 15-16, 16-17, 17-18 and 18-19 with phosphodiester linkages in positions 3-4, 5-6, 7-8, 9-10, 11-12 and 13-14.

13. The method of claim 11 wherein said nuclease resistance is achieved via inclusion of a 3 or 4 unit overhang precursor where the units are linked by phosphorothioate linkages to each other and to the nucleoside in position 19 and optional phosphorothioate linkages between nucleoside positions 2-3, 4-5, 6-7, 8-9, 10-11, 12-13, 14-15, 16-17, 17-18 and 18-19 with phosphodiester linkages in positions 3-4, 5-6, 7-8, 9-10, 11-12, 13-14 and 15-16.

14. The method of claim 2 wherein said AGSD modifications are selected from a group consisting of LNA, HNA, ANA, CRN R monomer, CRN Q monomer, HM, FHNA, CeNA, F-CeNA, cEt, 2-thiouracil, 4-thiouracil, pseudouracil and 5-methyluracil.

15. An in vitro method of improving an RNAi effect in vitro or in vivo against a target nucleic acid, said method comprising;
(i) obtaining an oligoribonucleotide sequence which specifically hybridizes to said target nucleic acid;
(ii) introducing one or more accommodating guide strand design (AGSD) modifications and other chemical modifications into said oligoribonucleotide, thereby producing a modified oligoribonucleotide, wherein said modifications are effective to modulate at least one parameter selected from the group consisting of
a) enhanced resistance to 5′ and 3′ exonucleases and endonucleases in vivo;
b) enhanced formation of a C3′-endo conformation in one or more flexible sugar moieties in said oligoribonucleotide strand comprising said AGSD modifications;
c) increased potency in vivo and in vitro;
d) reduced steric hinderance of strand interaction with RISC machinery via omission of moieties which project into major or minor grooves of duplexed RNAi triggers while maintaining RNAi activity;
e) reduced off-target effects; and
f) enhanced activity of the RNAi mechanism within target tissue in vivo relative to RNA strands lacking said AGSD modifications; and
(iii) contacting a first population of cells expressing said target nucleic acid with the modified oligoribonucleotide of step ii) and a second population of identical cells expressing said target nucleic acid with an identical oligoribonucleotide strand lacking said modifications; and;
(iv) determining the effect of said contact of step iii) on said parameter, parameters being affected by those strands comprising said AGSD modifications being identified as AGSD modifications which improve RNAi effects in vitro and in vivo.
